SEQUENCE LISTING (355 sequences)

SEQ ID NO: 1 - DNA, 38444 bp, Homo sapiens
  source 1..38444: mol_type = other DNA
agtcagccctgctgccagccagtgccgggtgctggggactcagggaggcccgccgggaccactgcgggacagtgagccgagcagaagctggaacgcaggagaggaaggagagggggcggtcagggctctcaggagccgggtcctgggcaaggcgcagccgttttcaaattttcaggaaagcggtcggctcacactcgagcagtaaaaagatgcctctggggaggaggcccgtgcagctctccgggcaatggtggtggctcggcctagagaggcggtagtggaacgcagaccctggtgggggaatgacatcaagggaggagacgggcgggaccccagatttctgcctgtgggcgatggaagtgaggttcactggccagcggagccggacacagaacgcgcaaaacgccgtgtaggcctggaggagccgaagagcaggcggaccccctccgcgggggaacagtttccgccgggagcacaaagcaacggaccggaagtggggggcggaagtgcagtgggctcagcgccgactgcgcgcctctgcccgcgaaaactctgagctggctgacagctggggacgggtggcggccctcgactggagtcggttgagttcctgagggaccccggttctggaaggttcgccgcggagacaagtgagcagtgagtcgcagtgaccctacaagtggttcttttacccgagcggctcgtaggcgcgttgcggtttttcgaaactacagctcccggcaggccccaagccgccctcggggccgcgggtcggcggattggccgcgctgcattttgggacctgtagtttcctgcgctcgtggcgctggcgccgcggcgttggctgagcccttgaccggggctggagggaagggccgacattcagtgtgtccgcgtctgttctgttagtcccagttcccgggcgggattgaggcttagagaagttgagtgatttgctgagggctgcacgggttggcatcccggcatgctctttcgctactttggctgcatctggttgcccacccgggcggatggggaatggactccagccagccaggagggcagagggctggagaggcagggccggaggttcagaccctccgctctgacgttgcgcctggtgaggccgggaggggtgccgcttgcctcttcagccctcacgctcttgtggaagtcgcggaattactgcaggcggaacttgcagcactgtgggcgtcttttccagagaaggacggagttgtggggcgggaggataaggcaaggcccagccacttcgcatcttcgccccgccagctcctcgagatgggatataccagggttgctctccaaccctctccgcaggagggactgatggaaacgcctgggaaagtagcccggtacccacaaaggctgtctacaaacagagtcttactgtctttcccaggtctgtgccatagggattctcgaagagaacagcgttgtgtcccagtgcacatgctcgcatcgcttaccaggagtgcccgagaccctaagatgttcggagtggttttttcgcacagacccgaatagcctgcccctcagccacgctctgtgcccttctgagaacaggctgatatgcccaagatagtcctgaatggtgtgaccgtagacttccctttccagccctacaaatgccaacaggagtacatgaccaaggtcctggaatgtctgcagcaggtagagcacaggccccgaggaaaggactgcgggtgggtggagcttcagccaggacggggtgtgcttccctctcccggcccattccagccaggcccctccgggccagaggcagcgtctgtcataaaaagggctggtgttccaggtggggtcagagagaggattgacaagtaaaaacgatcgtcctttgaagggggccggcccctccacacctgtgggtatttctcatcaggcgggacgagagactgagaaaatgaataagacacagagacaaagtatagagagaaaagtgggcccaggggaccggcgctcagcatacagaggacctgcaccggcaccagtctctgagtttcctcagtattcattaattactattttcactatctcagcaagaggaatgcggcaggacagcaaggtgatagtggggagaaggtcagcaagaaaacgtgagcaaaggaatctgggtcacaaataagttcaagggaaggtactatgcctggatgtgcacgtaggctagttttatgcttttctccacccaaacatctcggtggagtaaagagtaacagagcagcattgctgccaatatgtctcgcctcctgccacagggcggcttttctcctatctcagaattgaacaaatgtacaatcgggttttataccgaaacattcagttcccaggggcaggcaggagacagtggccttcctctatctcgactgcaagaggctttcctcttttactaatcctcagcacagacccttcacgggtgttgggctgggggactgtcaggtctttcccatcccacgaggccatatttcagactatcacatggagagaaaccttgggcaatacccggctttccagggcagaggtccctgcggctttccgcagtgcatcgtgcccctggtttatcgagactggagaatggcgatgacttttaccaagcatactgcctgtaaacatattgttaacaaggcacgttctgcacagctctagatcccttaaaccttgattccatacaacacatgtttctgtgagctcaaggctggggcaaagttacagattaacagcatcttagggcaaagcaattgttcagggtacaggtcaaaatggagtgtgttatgtcttccctttctacatagacacagtaacagtctgatctctcttttccctacagtccttgagggtgacagacttaggagtgccttgggggcctctctgaggagcagctgatattcacgggtcaggaggaagcatttccattagaggggcagccggtggccagcctcacttggaaggtctttgaacctcgggggtgcagggaggtggcagtggtgcaggttgccttctcctgggttccttgaggtgccctcttgtacccggctcacacccttcccctccccgagtttcctgctcaggttcccgtctgagagcttgtatgtaggacgtcagataggacagcataaatgtttggatccagaaacgcagaacagtttcctattttgagacttgacacctaattagtcatcttactatttaagctgaaaaatagtgtcgtgttttgggtaacgttctgcaaatcgtttgctaatggcggctgagttgcttcacgccctttagggcaagagtgggacttgcctgtggacttctccgcggtcccacagggctctcgccacctggcagtggcctctgcatctgcaaagagctgcccgctggctgccgaagcttgtctcagggcagcttgtgtggcctcgcctcttcctggcttccccgtaacccttgctccgaactccgttcagaaggtgaatggcatcctggagagccctacgggtacagggaagacgctgtgcctgctgtgcaccacgctggcctggcgagaacacctccgagacggcatctctgcccgcaagattgccgagagggcgcaaggagagcttttcccggatcgggccttgtcatcctggggcaacgctgctgctgctgctggagaccccataggtgaccctagttcccaggcctctcctggcctcctgtggggatggttggcaagggatggcgctgagggtggggtgggcccatggggactcctgccgtctctcaagcagaactcaaggagaattttttagctgctgtataatttctcgccatcgtgggtgtaaacctagggttgggcttttttgctgaattagggcacggcagatgcccacttcacccatttttgataaaccagtatctggggtgtcagattcttggctgtctgcagggccgagttagccgaatgccacctgcctttgatacgtgagaacgttgtctgagaaccgtgacttctgtgcttgcttgtgtctggtcagcttgctacacggacatcccaaagattatttacgcctccaggacccactcgcaactcacacaggtcatcaacgagcttcggaacacctcctaccggtgggtcagacgagtttacacctgtctcggggtcctcaagagaaccagcttggcatggtgctgagtccacagccccatgctgtgctgtggtggagggtggtggtctttctagacgctcccccgaagtgtgcagagcgctggtgcccaggggtggggtgcggcctgggctgcctccaatgcccattacttgtgaggaagcagctttgcatctgtgtgctgaccttgggcgggcgtcctgagctcctcgcaggtgctgttgtagcagctgtgcagtaggtcagggctggcccccagtgcagctttgcacatgaagtaggaggaggccctgctgcttgtcagagcccagcagagtcttggtgttctgtcgggttcctgtggccggaccagtggcagggtgctgtggaagctgtcgaatctcctccctctgtccagtacccccgctcgtcttctagctccctcctacgcccgggccacgtttcagttatgctcacttcctctgaccgccgaggctcctgcgtgtctccatacagctcacgctgcagggccacgctgtgggtgttggagacagctcctcctcgacccacggtgctctctcccaccaggcctaaggtgtgtgtgctgggctcccgggagcagctgtgcatccatcctgaggtgaagaaacaagagagtaaccatctacaggtaggctcctgggctcccgctccggctcagtgtccgacaggcgagtgctgctgggtgtccagagccccaggctgcgctcccgctgggctagggtttgaagttcactgggggactgcaggggaggacctggtgggggtggggactggcttcggtcctttcttggccgtgcttcagctgcgcactctgcccttcctcccacagatccacttgtgccgtaagaaggtggcaagtcgctcctgtcatttctacaacaacgtagaaggtacaagcagctgggtgggaccagggtcgggttggagtgtgtgcagcctctcagggtggagctcagtggtgtcacagcctggttgtgcttgcccggtggggcggccagtgcggccatgtacctgggccctgtcttctgactcggggccacccatgttagacttctgtgtggaagagctcacacagtggtctgagacagccagccggcaagactgcctctggctggtgcctggggccttggattttgggaaggctccctccatttcctgatgagagggtctccctgcacctaacctgctggtgcaaacagtaggggttttgctgaacaccggctttctcttcggggactttgttgcttgcccagcagcaggtgctccagtgaccggccctcataccatcttgggagggtgtcctggaagccgtgtctggcctcccgcgaccctgccccgtgtgtctttttcctgtgctgaccttgctgcggaaaattatggccctgagtgtgactccaggctgagtcctgtgggtccaacacgggatgccttggggcctcttctggagacgggatgtgagtgacaggagccggccggggcagcttgccctgtgactgcacgtggccacagcctgtgagggccgggggtgcttctccacccacgtggctgcccctcgggtatgtcaagggcttctggggctcatcacggggtcctagagacagtggcagggtgcacccccgttggctgcccttacagtttctgtgacctgagggtggcatctgtgcagtcggcgcggtctgtgcttctgtgggatcagggttccctctgtttcctgcctcagttggggctcaagcctcaggtgaggtggccccggagcactcagaaggcatcggcggtcctgtgggctgctttctgcactcacgtttgctgagtgctcagtgtgccaggactgaggaccctgaagctgctcttgtatttagggcggcgctcccctggcagagactgagccaggtggtcccgcatgacccactaccaggcgtttctgggccctggcccttggagggacagggtgggcggaacatgggcctgcagggaggctcccgcttactggaggcatgtgctgtgttgctggagacatcctctgtgttgcttcttgttcgctgtggtttttggtctggtggcaccaaggaccctcagtcatcttgatgtgtggttgtccaggcctttttgttggtcctaagaaggggctctgcctttgtgcccccaggttccctgacaggagctgccggctcgtcccggtgatgcctgcaggacgtgactctgggacggggggttgggcagatgtgctgatggaaattctcaagcaggcgtcatttccgaggtcctcacctggatttccaggacaggagtgcctgctgggtgtccccagtcccatgcagcgggggtccttgggatagcatggaacgctgagcatgggcctggccggccgtggtcctggacaagggcagtgccccggtggctgctgggcctgggacctggtggggacgctgggcctggtacctggtggggatgctgggcctgggacctggtggggaggcctctgactgcctcctggtgctgcttccgtctgtgttaggcctctgggtattggggcccccatctgtctcctcctccaggcctgtggactcagaccaggaagacacaggccagcccctgcctgtcccccttggcttgggctctcactgcccgacctggcgggaggttgcctagccgtgaaccttcgcaccctgtctgccaccggacaggctgtgagggggtgtctgcagcacctgcaccggcctgagcatcttcagagtgggctgcagctcctggaggggtctgagaggaagggaggcaggtattttgggcgaatgaggagacagctggagagctggcacccttcctggcctgcgtcctgtgaggactctggttggggacagcaagcttggggtcagcctggggcagagcctctgggacggccccgcccctcgtgccccttcccctcgcagctcctgtcctcgccccgccctcagctctccgccaggcaaggtttggcaagtgccgctgtgcggcagtgcctgctgattggctggtctgttgctatggtgctgcccaggggtgtgcttttcctcccctgccttccctgctatccctgggagtatctggggttgggtcatcgctggtgtgtgtgagtgtgtgtgtgtgtgtatgtgcacgtgtgcatatgtgtgcgcttctggcctctgcagctgagtcctggccctcggggggcctggcacctcctggggacaggcacaaagcagccatgatggagtcgggagctgggggaggccccattgccccacgtggctgccctgtgactctggggtgcttgttagaagaggtatctggttctgtctgtgtttaagcaactccctaaggaattcttgtggttccagtttggggggcctgtactgtagaggcaagggaggggcaggacatcccccagactctgacttctgaagccttttctgcccggggcctctccgccagtacaggcagtgtcctttgccagggctgccatgctgcagaggggagtgggccactgtttagcccaggaaaacctggctctcccttagctggaagttctgggcctgttgtggttggcagggaagctgagtgacggtgctaatcacaggggcacctgcaggggtttgtgggagatgcctctgtgggttggggcgataggctgaggggctgttcttccctgccctgaggagggctgagtgtagccgccactcctgtcctgtcttgggctgtctcggagaggatgcgtagaaccctcgggatcctgctggcctccgtctggtccaccctgaacctcaggccttctgggggcagaggaggattccctcaggatcactcgggtgggggcctctcttgggcacctgagaccctcagtgggtgctttgtggcgcgttcacggttggtgggggacgcccagccctgcccgccgtgtaggagccgttctgtcctgggcatccccctgtggtctgggacttagtggaccctgagggtgtgtgtttacccctgcctcacacctgcagaaaaaagcctggagcaggagctggccagccccatcctggacattgaggacttggtcaagagcggaagcaagcacaggtgagacccctcagtgaggccacgaccactgtccttccatggcccagctctcctgtgacctgtggaggcccggatatatttcttcacttttctttgttcctttttaaattatgaaactaaccaccattcagtacgaaaaagtttaagcagctctgaggaagatagagtaaaaaattgtctccctcttccctggccctcagccatccccggtggccaccgtggagtgtggacggagccctgcaggcctgtgtctgtgcggaagcacgcgcagttttgtctgcacagactgtcctgcagttggctgttttcactcagcgttgtgggtatagcttcccatgctggtgctggcagctcggccttgttcttttgaggacagcagatgtctcctatgtctacctcttacagcttcagagattcaagttataataaagctcttcttatattgagggggaaacctccctcccccttttttttgaaacagggtctcgctctgctacccaggctgcagtgcagtgtcacagtcttggctcactgcagcctcagcctcccaggctcaagcgattttcccacctcagcctcccaagtagccgggactgcaggcacacaccaccatgcctggttaatttttgtattttttgtacagacagggtctcactctgttgctcaggccagtctcctgagctcgagagttccacctgccttggcctcccaaagtgctgggattacaggcgtgagaccccatgcctggccagctctttttttttttttttttttttttgagacggagtctcgctctgtcgcccaggctggagtgcagtggtgcgatctcggctcactgcaagctccgcctcccgagttcacgccattctcctgcctcagcctcccgagtagctgggactacaggtgcccgccaccacgtctggctaattttctgtatttttagtagagacggggtttcaccgtgttagccaggatggtctcgatcttctgaccttgtgatccgcccacctcggcctcccaaagtgctgggattacaggagtgagccaccgcgcccggcccagctctgctttttcttagtggttctgcgttgtgtttgtttctatccaggaatagggttggttttacttttccatcgagtttttaaagagacgacgatttacatggtcggaaactcacgaggactccccatcccttggtcggaaactcacatggactccccatcccttggtcagaaactcacgtggactcccatccatcccaggcagcagcttcccacctgggccctacgtgcaggatgagggctccttccgggtcagaagacatggcggcctcggggcaccgtcccctgcatggggtgctcacaggatcttctcctctctccttcccagggtgtgcccttactacctgtcccggaacctgaagcagcaagccgacatcatattcatgccgtacaattacttgttggatgccaaggtgggggctcagtcctgtagctgacgactcctgatgtccaggggtgtccctgggcttgggaacagctgtccgagcctttgctgcttcagggccttagatcagcaggcctgggtgggaggactcacctctgtcactgggcaggggctcaacctggccagacacacttgtgagcagccccaggccacaggtcagttttctgagcagtctgggagcgggcaggctggtgggagtgaggagagacctccaggctgtggtccataggccagtgcccgctcttgatcctgacagctcaggttctctccttcacgtcaggccatgggaggcaccgagaacacaggaagcccactgactcccctcttcccagcgcgtgcccggccccacactcactccccctcccagcatgtgcccggcttcacactcactcccctcttcccagtgcatgcccggccccacactcactccccccacagcatgtgcccggcctgacactcactcccctcctcccagtgtgtgcccagccccactcccttccgccccgtgtgcccagccccacgctcactccccccgccagcatgtgcccggccccacactcaactcccctcctcccagtgtgtgcccggccctgctgccctcctccccatgtgccctgcttttgtgccccacactttttacttagtgcaggtgggatcacacgccacgggtcaatggtttgtgtgttcacgtgacgatggcgtggtgacgtttccagatcccgtcgttggttcgctcattctcggggtgtatatttattgagagctcatcatgctgggtgctattccaggcatagcaagactggcttcactcacatggagctttgattctagtggtggggacaggtggacagcaaaagagtaagcacgtgagctgatgatactgaagggaaatagagcagagggaggaggcggagaccgagccaagcgggcccaagtgcgatgtcggcgggaggtggggaatgctggtgggtctgaggggagcctcagcaggtgcagcagagcaagggaagaggtgagtgggggcggctggggggccgactcctgggaagctgtagcagaaccccacagagagctggtgaggtttgccgtggttgtgggtgactcggtgctttgagccctggctgcccctgggaaccatctggagagcttctaacccaaccaggcccctccctgggacagttatatcacagctggtaagccgagtctaacactttcacggaaacgcagaagatctaaaacagcaagatgaccgtgaagaagaacagagctggaggactcacctcgctggtttcaagactcctctaaagctgcaggagtggaggtggagatggcccagctcaggcacaggcctgcaggccatggagaaggcagcaagctcaagctgacccacacgcatgtggtcattgttttttttttcagttggaatctcactctgtcacccaggttggagtgcagtggcaccatctcggctcactgcagcccccgcccctaggttctagcgattctcccacatcagcctcccgagtagctgggattacaggcgtgcgccaccatgcctggcccttggtgattgttttttgacaaacatgccaatttaattgagagaggaaatgaaggttgatttctggttttctgaaaaaatggtgctaagaacagctggatatctgttcggaaaacagtgaatcttaactcttgttttaccctgtataaacctaaatgtaaaagctaaactaaaagttatagaaaggaacatgggggaggtctttgcaactttggggtaggcagagatttcttagtatggatacacaaggcactagccatgaagaaaaacattaaaatttagacttcaccaaaatttaaagcttcaactctgtggaagagttgagaaaatgaaaaagcagttaaagaaagggagaaaatacttctttcaaaggacttaaaaaattttttcagccctcctctgatttgaaaggacctttgaccagagtatgtaaaattctcccataactaagcaaacaacccacttaaccactgggaagggatctggacagacgtttcaccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcgccaagatgggtggaatggccagttaaccactgggagagcatccggacagacgtttcaccaaggtggatggaatgaccagttgagcacatggaaagtcgcccagcatctccagtcataggagaaggcagattaaagccacggggagccgacactgtggtcccactggcatggctgaaattcagaagccctgagtgtggcatgaggatgtggaacagctggatctcatccatcgctgtgaagttgtgtagccactccacaaacgtgtggcaaacagccgagccgggagaagggaagacgtgttcaaagattcatatgtggccaggctcagtggctcacgcctgtaatcccagaactttaggggccaaggctgggggatcgcttaagcccaggagtttgagaccagcctaggcaacatagggagaccccatctcaaaaaaaaaaaaaaagaaaaaagaaaagacttcagtgtgcaggtttaccagagttttgtttgcagttgccaaaactgggaagcagcccgcgtgagcccatccacaggtgaatggacagaccgtggtacccgaacactaacagcagccacgggcgtggactgtggtcacacagcagcagggagccgatgagtctcggacatgctaacccagagaggcccattgaggaggacctactgttttttgtgtttttgttttttgttttgaaatggagtctcgctctgtggtgcaggctggagtgcagtggtgtggtcttggctcactgcagcttccgcctcttgggttcaaacagttctcctgcctcagccttccgagtagctgggactacaggcacccgccaccacacccggctaatttttgtattttcagtagagacggcagttcgccatgttggccaggctggtcccaaactcctgaccttgtcatccactcactttggcctcccaaagtgctgaggttgcaggcatgaaccaccgcacccggctggacctactgttttattccatttatgtgacactctattaatagaaaaggcaggggtggggctggtggttatatggtgcacataactgccagaactcagtacacttaaaatgaacatcttaatgtgtgaaatttttttttttgagacggggtcttgctctgtcacccaggctagagtgcagtggtgcgatctccactcactgcaagctctgcctcctgggttcacgccattctcctgcctcagcctcccgagtagctgggactacaggcgcccgccaccacgcctggctaattttttttttttttttgtatttttagtagagacggggtttcacagtgttcgccaggctggtctcgatctcctgacctcgtgatccgcctgcctcggcctccgaaagtgctgggcttgcaggcgtgagccaccatgcccggccaatgtgtgaaaatttaaaagtaccaaagctggaccccaccccagattgctcccatgacactctgtgggtgggacctgggagttgggttttgttttgttttgttttgtttttgagatgaagtctcactctgtcgcctaggctggagtgcagtgacacaatctcggctcacattaacctctgcctcccagatgaaagcgattctcctgcctcagccttctgagtagctgggattacaggcacacaccaccaccccctgctaatttttgtatttttagtagagacggggttttaccatgttggccaggctggtcttgaactcctgacctcgtgatccgcccgcctcggcctcccaaagtgctgggattacaggcgtgagccaccgcgcctggctgggagttgggtttgtaaatctccctgagtggggctggggcagggaactgctgggtctgggtcttcctggctcctctggtctgtggcttcctgactgcggtggccgggggctcccagggcatcgtggccgtctgtcttgctgagcgtggcacgtgcctttccatgctgtggaggagcgtctcccggtatggcgaactgctggttagggtggggcggtgttgccaggtcatccaggtctggcctctgctctcgacatcgccggcgctgttgctcatctgcgcttgtgatgttcgatgcctgctgcacatgtcttggcttccctctttcccggcctctgtgagctccagcgctgcgtcccttctcttcctcctgtagagccgcagagcacacaacattgacctgaaggggacagtcgtgatctttgacgaagctcacaacgtggtgagtctccgctggcctcctaaacacctcctattgcttctggcctttttgtcaagagccacgcaaacctttctggaggggctctggccaaactcctgaagccctaggtgcccaggactggggactgagcacaccaggagcttctgccaccccctcccgccctgatccgatgcctctgctggggctggagactggccagctgggccagggacctgcccgtcaggcgcagggcccccacaggccgctcaccagaccctttccctccagccagctcggggtcagcctgggccagggctgtctcctctgccctcggcagcagcaggcttgtggtcttgcctgcagtgtctctgcccttccggccacatggcttgagactgaggcaggagaatcgcttgaaccttggaggcagaggctgcagtgagccaggatcacaccactgcattccagcctgggtgacaaagcgggattctgtgtcaaaaaaaaaaatgttgactgggcgcgctagctcatgcctataatcccagcactttgggaggctgaggtgggcggatcacgaggtcaagagatcaagaccatcctggccaacatagtgaaacaccgtctctactaaaaatacaaaaaaattagctgggcgtggtggcgtgtgcctatagtcccagctactcaggaggctgaggcaggagaatcactcgaacccaggaggtagaggttgcaatgagccaagatcacaccactgtactccagcctggtgacagagcaagactccgtctcaaaaaaaataaaatcaaaaagaataattggcaattccagtgaaataattgtttgtttgtttgttgagacagggtctccttctgtcgtccaggctggagttcagtggtatgatcttggcccactgcaacctccacctcctgggctcaagccatcctcccacctcagcctcccgagtagccgggactacaggtgcacaccaccacgcccggctaatttttgtattttttgtagaggcggggtttcccagcgttgcccaggctggtcttgaacccctgagctcaagtgatctgcccaccttggcctcccaaagtgctgggattacaggtgtgagccaccgcgcccggcctgaaacaatcgtttctaaatattggtgtgggccacacagtcatgtttggacctacttgtggccttttacagaccccaggccaaggctttgggaacttggctgtcagcctcctgtgccttctgcacccccaccccatttctgctttctggaacccccgatcctgtcctgttctgtggtgattcgggtgtgcttgggctctaggagaagatgtgtgaagaatcggcatcctttgacctgactccccatgacctggcttcaggactggacgtcatagaccaggtgctggaggagcagaccaaggcagcgcagcagggtgagccccacccggagttcagcgcggactcccccagcccaggtgcgttcatagccagactgcttggtcctgaggcctgcgctgctgcagggtgagccccacccggagttcagcacggactcccccagcccaggtgcgttcatagccaggctgcttggtcctgaggcccgtgctactgcagtgggcagcctgccctgtggctgtgtgtggtcggcctgggcaccatctattcaggctggcactgcagggcatccgcttctctcagaggcttcttgggtgtgaattcttcagggtcctgtagcctgtggaagggctggtattgttcagtagttctggtattttccaaagacctatgtcttctcccagccagtatcaacttggcctctactgtgtaaaactggaaaactctactttgtgaagctgagttgggagcatcgcttgaggccaggagtttgagaccagcctgggcaacatggcggaacctcgcccctgccaaaaaattagccaggtgtggtggtgtgctcctgtggtccaagcttttccggaggccgaagtgggaggcgtgcttgagcctgggaggcagagcttccggtgccccagatgactccactgcactccagcctgggcggcagagtgaggccatctcaaaaaaaaaaaaaaggaaaactaaatatattcactgtaagggcattttgcatctttaaatgacccacaaatctggcatgcatcagctgctctgcctgtaggttccttcccagtgtttgtccagaggtgtatttccacacagcgctagtcacggcatacgtggaaaacgtggaaacccttcatggatgttgtcagttggtctatattttctttcttttttttttttttgagatggagtttcacttttgttgcccaggctggagtgcaatggcgcgatcttggctcactgcaacctccgcctcctgggttcaagcaattctcctgcctcagcctcccaagtagctgggatcacaggcgtgcaccaccacgcccagctaattttgtatttttagtagagatggtttctccgtgttggccaggctggtctcgaactcctgacctcacgtgatccacccgcttcggcctcccaaagtgctgggattacaggcgtgagccgccacgcccggcctttgtccatattttctacatggcttctgtaaacagctgactaggagtctgtgtgaatatcttcataggttctgctgtgacactacttgctcgtgagcatctccaggtgtaaacagcatcagcttcccccattttcctttaaaatcgcacatgtggacggacaccacggggaccctggaccctggggagccccgtcctcacccttctcaccaggatggctgcttggtagagagtgagtttgcaaagttggcatttgtttagtacagaagttatcaggtgttctggctttagaatccctttatatatatatatatatacatatatttaagtgacagggtctcactctgttgcccaggctggaatgtggtggtacaatcaaagttccctgtagcctcggcctcctgggctcatgggatcttcccgtctcagcgtcttaaagcgccgggaccacaggtgtgcaccactgccaccggctctcaagattgccacgcagggagttgcagtgggggaaggggttcctgggactttgaacgctccacctccctcctctccacagtcccccaaccccacctctctaacggggtggacggccgcctctttccatccttcgcttggcgcagggtggggagagtgacaggtctccttccctcatctcggcagctgccatttcatcgcttacataacgtgggagaaacatccacccacccccaggcctgtgtgaacatcaccacggggccttctccactcttcagttttgttagttacttgatgtgcagggctttttgttgtaactagtgggggacgtgtggtggggtgggcttctgccatctcattcaggaccagaacttcagttttcatccctatctgttcccccacccctttggagatggggtctcactctgtcacccaggctggagagcggtggtgccatcacggctcactgcagcctccacctcctgcagcctccacctcttgggctcaagtgatcctcctgcctcggcctcccaagctcctgggactacaggcgtgtgccactgtgcttggcagggtccattcttttcctcacactttatttattgaagagcccaggccgtttaccctgcagagtcggaatctgtacaggaggggcagccacacgagttccccggtttactctgaacttaggtggcttgagggccccagttagactgcggccaccgtttgccgggctccagatgggacgtcctttctatcagaaggctcacagtatctcctttcccgtttcttcccatgtgaacattgttgctgctgaacacctgaatatgttaatcactgggggcttgcaagatggcagtgtgctaattccatcatctagtcagttagcaggaataacttaggaccacgccctgcaccatatcagctatgtggtgatcccattcacacaggaaaggtgggacaaatgctgggggtgggccgggtgtgctgtctcacacctgtcatcccagcactttgggaggcccaggcaggcggatcacgaggtcagagattgagaccatcctggccaacacggtgaaaccccgtctctactaaaaatacaaaaaaattagccaggtgtggtggtgcatgcttgtaatcccagctacttgggaggctgaggcaggagaatcacttgaacccaggaggcggaggttgcagtgagccgagatcgcaccattgcactccagcctggcgacagagcgagactccgtctcaaaaatcaatcagtcaatcaagtgtcatcactgaatgtttgtgtgtgaacgtggggattggtcctgccccatgctccctcctgaatctcactcctgacctcagttgctgcaccttgaggtgttttcatgtgggctcttgtgtcctgaccccggcggttgtggcctctttgctgtctgggagtcaggatttttcacactcatgtcctgctccagacctggaatcagccaagtctccaagaagccctgctttcttttcctgcaagacggtatttcaagacccgccatgcggcagcgggttggtcatggttactgggttggtcgttgttactgggtgttttcgtggagatacagccatacgcacaggtgtgttcacaaatgttaattctaaaggtcaaacacccggccaggcataagggctcagcggtaatcccagcactttgggagaccaagactggtggatcacctgaggtcaggagtttaagaccagcctgagcaacagggtgaaaccccatctctactaaaaatgcgaaaattagccgggcatggtggcgcacacctatagtcccagctagtcgggagacagacacgagaattgcttgaacctgggacatggaggttgcagtgagcagagatggcgctgctgcacccctgcctgggtgacagagtgacaccctgtctcaaaaatgaatagataaataaagataaaacacctgctcctcttggtgtctccagtttggatttggcctgtgtagcctcttccttcgcctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccctgttggtggatttggcctgcacggattctgtgtggcctcttccttcccatgttggtggatttggcctgcatggattctgtgtggcctcttcctttccatgttggtgtccttttttccatgccaggaatcctggttctcaagggcggggttgttggcacgagcgtgatgcagactgcctttgctgcctttctcttgcccagggctgaacatggagctggaagacattgcaaagctgaagagtaagtgttgccctccccgcctccttgcagctgggtggggcctcctccttgcgaggaggtgggtgacacctcctcgacccacagtgatcctgctgcgcctggagggggccatcgatgctgttgagctgcctggagacgacagcggtgtcaccaagccagggaggtgagaggcggggagccagccccttcactgcaggcccagcctagagctagaaacgggccatggtgcagtcctgggctgtcacatcacgagtgaggcctgttttcaggcctgttttccctttttgagacctgggaggagcacctgctttgcatgatctggttgctgagatgttgagaggagcagcacacactcccacgggacagcacacagccccccacggaacggcacacacacccatggaacagcacacacactcccacgaacagcacacacactcccacgaacagcacacacactcccacggaacagcacacacacccacggaacggcacacacacccacggaacagcacacacactcccacggaacagcacacacacccacggaacggcacacactcccacggaacggcacactctcccacggaacagcacactctcccacggaacagcacacacactcccacggaacagcacacacacccacggaacggcacacactcccacggaacagcagactctcccacggaacagcacacacactcccacagacagcacacacacacccacggaacagcacactctcccacgcggggccgctgggtttcctgcagtttctcctcctccaggcctttccctggaccctggtccagtccgtcatttgagcacaggtgcctgttagaacgagaccttcttgttaggacgatgagtgtcccagccaccacctcttttggactccgggaggcctggaacgttctgaacgctccgtggggctccagtcttctccgcagccagggcagcagggtttgctgtctgtcctgcaggcagatgaggagtcagggctggggcctgtgtgggggctctcctgagcgcacagccgccgaggtggagcgtgttctgcctgagcgccgacctggtcgggggaatcccagttgcttccaggtggagccactgtcctcagcgtaatgctcaaggctctggcctggctcctcggccaccctgcaccctcagggtcccctcctgtagcttctgctgccccatcactgtcactctccaaagctttggggactctgcccagagccaccgcctcccagaagcccctgacaacctcttgacgaccccctagtgaccccatccctcccctctgacggcggcccctgctctgaggcggcttcttttcctcggtgctgttctcgtgctggccaggcctcctctccccacctggaggctcctgagggcggaggcctctcacctccaatgctggcgtcccctggagggctgaatttgtttccgagggaaggaaacttccacagttgttgccttcagttccaaagctgcagcctgatttccccctccaggctcgagcctgttttcttctcggcagctacatctttgaccagtgtcgtcccccctcaggcccgagcctgccttcttctcctcagttcccaaagctgcagtctggtccccccgccaggctcgagcctgccttcttctcctcggcagctacatctttgagctgtttgctgaagcccagatcacgtttcagaccaagggctgcatcctggactcgctggaccagatcatccagcacctggcaggacgtgagtgctggcacggggtctttggtgcgggcaaatgtggcgtagggggtgcagcaggcctccatcttggcagtcagggctcccctggccgtcacctggccgtcagcaggaacaggcccacagaacctcatcttctgatcggggcgtggaggcgttagtgccacttgccagctgccgtagagcctgtcccagttctgcagctggcggcttcgtcctacagcctcatcccattattctgcttttgagaaagagcagcccaaggccctagctggcttgtggggcctctggcttctccacaccaccccgagttctgcttctcagagttgtggggtccagaggctttgcccagaggcggtgtccccatgggctgctctggtttgagacgccgggcccagcggggtctctcctctgctgcgctcccgggtgctggggagggtggcttttgctgcttcaacccttaggcgaccatagagcctcttttcaagtcccactgacccccttggagactctgtccctgcctggcttctctcctggctgctgggaagagcaggcgaactgcccgccctgaatggatgctgcgctccaccctgggccccccattgggcaggagatggagcttggcagtcgggctgagcgggctcatgctggaagggccggggctggggtcggggcctcccctgcctgcagtgtgggtgtcagcgccctgctgccctccaggtgctggagtgttcaccaacacggccggactgcagaagctggcggacattatccaggtggggcctgctcctctgtggcatctccttccctgatggaagccgggcgggtgccttctcctgctgtattagttaactgattctagacttggggatgggagaaaggcccctacaccacctgtttctgattggcaaactctcggctcctttccagtgccctaaacccacactgggcctcctgcagggatgggggaggacgaggtctggtggcacatgcccagggtgatgctggtgagggaggacgcaaaggacagtgggggccggggagccgctcctgccctgtccgggccctcaggccaggggggacccactgctggcagccccagcagccccagctgcacgcagatgaagagctctggacacacgcggcttcctgaacagcttctccagggacagacaaatggggaccctgcaggttcccggcaggggtgtccctgggagcccatgattgggggtgcgaccctggcccccttctcattggccccgtcctgtcctgcaatgcccgtcccatgtgaggtctgcttctggctccatgcctatggcagcacctgctttccctggcgtagaggtgcttgtccggtttgtggagggcacgccccattttgggtgctctgggcacgttgcctctccggggcctcggtggcttttttagaagcagactcagaagtccctgactggggaagccaaggcacaggtggctgtgtggagccctgtgaggcctcctctgtgctgcccacgctgtacctgctggccacacgagatcatggcagggttaggcagggctgcccagcgctatgacagcttcatgagtgtccatctggcctgtggggtgcttgagctgggggaggccgcagaagaaccctgggatgcatggctggcctgtgcatgctgctgggcatggagctgcagatcccggaacaagcaggcactgccttctccttcacagacgcagctctgagcgggggcgagacctgggcagggaccaggtggggtgggcacagggtggtggggcccaggctcagccctccctccactgtggccgtctctgtggccagtgacgccacagcctgtgtcttctctgtgcggtagctggggctggaaggacagcactgccttgtcctcccaactcctccccaaaggcacggtgggcatcccaggcccagacccctctgtctgtggctcctgcctgccaagggctgctgtgctgtcccgcatggagtgtggttggctcttcaagcaggaggccgtgcacctatcaggcggacctgcttccatgtccctgatgggtcactgcaaagcacctccagcacatggccaggcgaggtagccctgcagcccagggcctggagggcaggtgtgagctggcccgggcctgtccctccctggaatacagcttcccaggctcccacttatggagaagtctcctccacactatggaactgaatcctagaatgtggcttctgaggttcctacactcgaactgaatcctggaatgcggcttccaaggcttccagctatggagaagactccacactctggaaccgaatcctggaacgcggcctcccaggcccccagctatggagaagactccacactctggaaccgaatcctggaacgcggcctcccaggcccccagctatggagaagactccacactctggaaccggatcctggaacgcggcctcccagcctcccacttaaggagaagtctccacactctggaaccggatcctggaacgtggcctcccaggcccccacttaaggagaagactccacactctggaaccgaatcctgcacactccatcggtttggaatttcctttggctgctgctctaagtagccgctggtggatgactcagcttctgccagccctcgggtgcctggaggatgagggactgcacacagtgctcacccgcgttggctcctgagcccctgcaggtgtgggcggtgcccatagggctggtgctgggttgggcctgcagccctgagtcacaggtgaccctgggggcagagtggggccagtggccccaggaagaggatgtgggatgcacagctcagctggaggcgaactccaggcagggtcaggccgtgtgctcggaagtcagggcttagctggaggcaaactctgggcagtgctggcccgtgttggggaaccagttgcccctgggcccccgtgagactgctgggtcctcatccctctctgcctgaggccggagctgccctgggctgaggcacagggggatttgtggtggtgtttttttgagaaagggtctcgctttgtcaccccggctggagtgcaggggcttgatcacagctcactgcagcctcaacctcctgggcccaagtgatcctcttgcctcagccacccgaggagctgtgaacacaggtgtgcaccaccgcactcagctaatttttaaaatttttttgtagagatgaggtcttgccatgtttcccaggctggtctcaaactcctgggctcaggcagtctgcccgccttggcctcccaaagtgctgggattacaggcaagagcttccatgcctgcccagcagaaggcttttcgaaggaagctgtttcctgaggcagactcagccctgctcatggcagccaccagcgtgggggtgaacttgttctgttacttccatccccgtgggccaaatgctttggtaaaacacaaggccctgtgtttagctgtcttgacagtgaaaatggctgggaaggaaggaaggaacggaaggaaatttctctctccttctgtgcgtacccaggcacgtgcacatgcatgcagagtacgcacacacgcacgcacgcctgcacaaatccacgcatgttgccaagtctctgtgttccagccgtggtgtctgccccccggtgttctctagttcggcttctccgcatttctgtgaatgattccggcttcttggtgttcccagcagaactccctcaagtctgcggcggggctctgacggcggtggcttggctgacatggccacattgctgagcctgttgggggctttgcgttcctgttctggccgtttttggctcgttttccaggaacggtcgtcacgcgctcctctcctagtgcaggcatcattcctttcccattgatttgcagggttctctgtaagttctgaggatcccatatacatatactctctgtaagttctgaggatcccatatacatattctctctctaagttctgaggatcccatatacatattctctctctaagttctgaggatcccatgccgacatacatattctttccttgtctcatgctggtcattttttccattttcatgacaggtttggtgaacacatgtttccttgtcagatttttgttctgagcttgtgcctcccgaccaagatgctaaaccgggtcttgtgtattctccaaactgcactgtagagtgacggagctttgtgtctgggcctccatgccttctgacgtcacctgtgggggtgtgaaaggcagactctaccttgatttttcccagcacgccacaccggtggttctgtgcgctgaccgagcggctcggcttcccccaactccactgggcacctgccacacttttcctcatgtttttgttcactgtggttttgtcgtaagtcctggtgttggcctgaaccaatttctttttgtttgtttttgagacagagttttgctcttgttgcccaggctggagtgcagtggcgcgatctcggctcactgcaagctccgcctcccgggttcacgccattctcctgcctcagcctcccaaatacctgggattataggcacctgccaccacgcctggctaattttttgtatttttagtagagacgaggtttcaccgtgttagccaggatggtctcgatctcctgacctcgtgatccgcctcccaaagtgctgggattacaggcatgagccaccgtgcccagcctgatatttttagtagaaatggggttttgccatgttggccaggctggtctcgaactcctgacctcaggtgatcctctcaccttggcctcccagagtgctgggattacgggtgtgagccaccacgcccggcctcttgttcttttgaaacctgccctgacgttttttccatagtgcatcttggagtcagcgtgtctacttcctgtaaaaatcttactgtgattttgactagaatgtgttgaattcctgttttttttttgagtcagggtctctctgttgcccaggctggagtgcagtgggaccatcacagctcactgcagcctcaacctcctgggctcaggggatcctctcagctcaacctcccaagtagctgggaccacaggcacatgccaccatgcccggctaggtttttttttttttttttttggtgaacaccctggggttgcaccatgttgcccaggctggtctcgaactcctgggttcgggcagtttgctcctctcagcctcccggagtgctgggattacaggcctgagccactgcactaggccatgttgaatttctagattaatttggggccctcaggggcacagagaggagggctgggccagttggcgggaggagaggcccctcgggctgccgcattttcagtgcatggagatggcctatgttgggggaacacagagctcaccgggggtccctgcagggaggagaaagggtcaggcaggtgccagctcctgtccattggcctggggctgcatgatggcaggggccggtgaaccgatgacccctgggtgtcctgtgaccttctgtgtatgcggctgatgctgcagaaagtcgggtggcctcaggctcctgacggggctgcacttcctctgcctttcagattgtgttcagtgtggacccctccgagggcagccctggttccccagcagggctgggggccttacagtcctataaggtaggggccacctccaggaggcaggtggagggcagcccttgttccccggcagggctgggggccttacagtcctataaggtgggggccacctccaggaggcaggtggggctgggggtcttctggtcctaaaaggtaaggggctgcccccaggacatgggcggggcctccacactcctggtcctgtcccctccaggtgcacatccatcctgatgctggtcaccggaggacggctcagcggtctgatgcctggagcaccactgcagccagaaagcgaggtacagacctgggcccacacgctccccgcccgcccgggtgcagtgcccggcaccaccatgccacaggctaggcacatgcccagccgtggatctcctgcccccatgggcctggccaccttctccatatccaggccaatccagagcattctcctcactgtccctctgaagattggagttactgagagacgtaggagatggcctgatggcaccgtgacctgcccagagtcacctggttggtggtggcagagccacagcccagccaggcctccctgctgggacacgctcgtttatgccgaggccgtcagcacagagcctccacagtgaggcacggctctgcctgctgcctccacgcagcgcctggccgggccaagcctcagggtcacatctgaagggggcccggctggccctgttgtccgaagcccctggtgcgctcagccccgaggccccacgtgccttcttggcttcctgtgctccgtggcgtcttcgagtcggtgctgccggggacgctgtgtggatggggtctgtgagtgtgccctcggctccgtgtccggagccctgtggttcttggggtgtatctggccccacccccactgcgtggtgtccagggtggggcttcacggctgcagctgcgggagctgctgcccctgccttgtgctccagtggggccttgcctctgggcttggttcgtccctctctggaacattctttctcagctgctgtccgacccatggtggcatgacgtggccctggctgaagcagcccttgtgcggttgctgtggttgggtctgcctggccgagccggaagggaagggctgggagggcgtcagggtggcgtggcttgacccccgctcggtgatggtcctgcagcaaggcctctcccagcaggaagcgtccatcccggggggaggccggcgcccctcacgcagttggggttgcgggaggcagtgcgtgcctgaggcagccggtgcacagattccaagggcctggaatctgtttgttccattgacctctgatgtcacttgacttctcagaagcagccactccctgcactgggcgtttgtaggaaatgagctcctggaggagggggtggggaagttcccccattgcagggcacactcagccccaggaaggaaacgtgcctcgtccctgctgactccgaatcgcagtcagagtcgttctgcttgtgccgtgttgaattcccggcatccggcatccagactcagcctcctccccaggccacggccgccgtggccagtcggtcaagcccttctaggaacttcctttgagctggcgcccttgttcactgctgacgccactcagaggcttgtgcacgtgtcctgcttccaggcagagctgggaactcgcaccccgtcttctgcacgcggccgtggaatgtcgggatgccggcgcttccttcccgtgtgctcttggcggggtgggcttcttgccctgagccgcatgtcacagtttctgcagaagtttagggttggagtgggctgacctctctgcaggtgtccccagcctctgcctggggtctgcctcctactcccaggaccccctgtcccccagaggggccccaagctggcaggctcacactcagggcagcctcctttgttctgacttctgcacagtgggcctgggtggctgcccgcggctcgcttgcttgatgccagtgggtggagagggtgatgggcagagaggcaggtggtcaggcccccagtcccgtcctcacactctgtgccctctgccgccccccgccccacagggaaggtgctgagctactggtgcttcagtcccggccacagcatgcacgagctggtccgccagggcgtccgctccctcatccttaccagcggcacgctggccccggtgtcctcctttgctctggagatgcagatgtacgggccacccctgccagggcctgagcaccggtgacacctctgacatcagcggggtggaagtggtgggggtccccatgagccgggtgctgggggtctcgggcctcgagggctaaaggggtgctggtgcacttccccactgtctgctccctctggccacgctcagccctttcccagtctgcctggagaacccacacatcatcgacaagcaccagatctgggtgggggtcgtccccagaggccccgatggagcccagttgagctccgcgtttgacagacggtgagggcctgtccctgggccctgctggggtgggaggtgggggagcactgaggcctgaggtcctgagcagtggcctctccggctctaggttttccgaggagtgcttatcctccctggggaaggctctgggtgagtgccctgaatgccccagctgtgcccatcctggatcctggacccctgctcccaagagctggtagggaaccctgcagacatcctgcccctgccttgaccccggcccctgcacttccaggcaacatcgcccgcgtggtgccctatgggctcctgatcttcttcccttcctatcctgtcatggagaagagcctggagttctggcgggtgcgtctcccctgtgttctgggcggggtgggtgagggcagggctggagcatgaagcaggcagtggtcacagctcctgcttgccctcatcggatcggcggcgtgaccagggctgccgtgtccctgcctcttcctcccacaggcccgcgacttggccaggaagatggaggcgctgaagccgctgtttgtggagcccaggagcaaaggcagcttctccgaggtcggcacttggccggggctctgggcctgctgccccctcgtgcctcccctgcctctcacagcttccccaaggctgaccactggccctgaccatgggctccggcggctcccgctgcctcttcagggctcctgcgtttccttcctggccctgagtgttgcctcttatcttacaaagcccccagcaccgggtgggtgtggtaacagtggccctcctgtctgagtagccctagtcggccaccctggccctggggttccccgtgttttctgggaagcactgagcaggcgtggggtcagcctgggatccgtgccaggaagaagcttccagaacccgattggccttcctggctaggacgatccttcatcttggagcatgagacctgggtctccctcatgggggaggaaggggctgggggggggctccaggctcagcctcaccaactttccttccagaccatcagtgcttactatgcaagggttgccgcccctgggtccaccggcgccaccttcctggcggtctgccggggcaaggtgagctctccagggccctctgccctgacctggttgcctgttccctggtgggtgcttatggctccccagcagactctgggccctgggggctgcccggtcccctccttgggtcccacgagagcgactgctggccctgctgggagcgtgtcctgctctgggcctgggcaggcaggatgggagtttcctggccacaagagttggaggtggcgtctgggagctgtggaccccaagtggggtcctgacccacagatggagcttcctcccacccctggttggggacggagcctcggggaaggtggctgggctgggtgtgggcaccagggagaggagcccccacggccccaggcagctccctggtgtgtcccctaggccagcgaggggctggacttctcagacacgaatggccgtggtgtgattgtcacgggcctcccgtaccccccacgcatggacccccgggttgtcctcaagatgcagttcctggatgagatgaagggccagggtggggctgggggccaggtgagttacagcagggtggggctggggtaaggcggtctggtgactgagcccccgccccgtggccaagggagcccccgtgaccgagccgcctcgccccacagttcctctctgggcaggagtggtaccggcagcaggcgtccagggctgtgaaccaggccatcgggcgagtgatccggcaccgccaggactacggagctgtcttcctctgtgaccacaggtgcgtgcagtccggtggcaggcgcggcgccaggggacacgcccacaccccactgggcccctggactctccttccccacatgaggccccgtctcctccagagcctctccggctactcggggtcagcgtggggcccctgcagcagatgagggtcttcacttcggtgaactgaacccttgaagcggctgtgggcagggcagcagggctatggccaccccccaggttcgcctttgccgacgcaagagcccaactgccctcctgggtgcgtccccacgtcagggtgtatgacaactttggccatgtcatccgagacgtggcccagttcttccgtgttgccgagcgaactgtgagttcctgcccagggaggggatgagggtgttgtccccagaggagccagaaatgggtccacccacccccatggttctgcagatgccagcgccggccccccgggctacagcacccagtgtgcgtggagaagatgctgtcagcgaggccaagtcgcctggccccttcttctccaccaggaaagctaagagtctggacctgcatgtccccagcctgaagcagaggtcctcaggtgcggacgggcagcgctgggtgggcggtgtgggggtggcggagcgggcggcgtggggcgggcagcaccaggcgcccagggcggaggcgactcacctggctttgtgcgcttcccctcccacctccaaaggctgcctctccctcctagggcagggcccccacgggctgcaaccctcccctacaggcagagaacgccccaggcaaggatgccccccgaggctgagactccccccaatagcagggaggacacccacaggcaggaccccaagtgctgggactctcccccaagaggggctttgccacaggcagggaccccagctggggccccccgtgggcttcactgcgcactcgggtgcccctgcagggtcaccagctgccggggaccccgagagtagcctgtgtgtggagtatgagcaggagccagttcctgcccggcagaggcccagggggctgctggccgccctggagcacagcgaacagcgggcggggagccctggcgaggagcaggtacagttccagggccttgggatggacacagaccctctgtctcctgaggccaacccgaccccgcccatctggcctcaggcacctccccacacacccctgtaaatcccctgcctggcaggcaggcgggcaagcgggcgggggatcccagctgcctggctgtctgtgggtcctccaccccacctcacccacaggctgctggctcccaggtggtgcatgccctggccctccgcgggtgccccccacatcactttggttctctggcgggtcagcttggctcagtgcactcaaggtcgggtgcccctgccactggctgcgcttgaggctggcctttctccaggaatgtgctgcgggtggaacccaggttccttcttccttggggccttttgccccagaagcccataattcctcaggccaacccgaaattttctccctgcttcctgctgggagccattcccctcttcctgcccatccctgcccttcaggcccctggagtgagctccaggtgcaggcaccaggcacctgtgtccccttcctgccagcccctcgctgtggtcggactgtcttccctggacctgctcttacaagtcaccacctgcgagcctcatgagccgctggtgtgacttggacaggaccaagttgtggcactgtcaccggggtgtgctgtgcccccctcccccgacctccatcttggctcagggctccttgggaccatcttccctgtgcgtccaggtgctttgggaccccagagtgtgtggttggggtctgtgtgtggttgtgagctgtgtcctcctcaggcccacagctgctccaccctgtccctcctgtctgagaagaggccggcagaagaaccgcgaggagggaggaagaagatccggctggtcagccacccggtgcgtgagctgtccctgcacctgtgccgaccaccatagacacgcatgggaacgcagccgtgggtgcccccagccacggctggtcccgatgggaccagggaatccacccccaggagctgatgtccagggcagctgtgatgctgacggccaggggctcaagtgtgtggtttcttctgcagggggctcatgagtcccagctggaatcaggccccacccttgggcaggtttggcatggggcctgcagcactgggcttggccctggcatttccctcaagtgtggatgcacacctgcctcatgtgagggacacagcccattcctagccttggatcaaagaacggagttatagccggagccaggaagccccctgcctgctggaaaaccccaagtgtggcggcctttgtccatgtcccttggcttctgggaagaactgggtggtgcccaggcagggctggtgccatcaggaagtgggtggctgctgaggggcctgggctggcgagggcctgggtggggagtgcctgggccgcccctgccttggtttccacgtttccgtgttggtctggggtgtgtagagagatgggcactgctcatccggaagcccctccttgtgcgctgccatcctgggagcctcagccgcatccgctgtggggcagggggcttgagggaggaggagagagacgggccatgcaggacccctggcttgaggcagagccaatctaccctttgcccattcactgctctcagttccctgccagcctctcactgtgtgacctcagacgggcccagccccacagctttcttcccgcagcccctccctatgtccatccagccagccagtttctcaggcagcagccccacctcggcagtcactgtcccagggaacgctcaatgttccaaggaaggctctgcagccccagggaccagatgatgaggctggccctgatggagcctcgggcctgtgtcctgcaggaggagcccgtggctggtgcacagacggacagggccaagctcttcatggtggccgtgaagcaggagttgagccaagccaactttgccaccttcacccaggccctgcaggactacaagggttccgatgacttcgccgccctggccgcctgtctcggccccctctttgctgaggaccccaagaagcacaacctgctccaaggtgccctggcttgcagaggccacccaccctgagggcagtgctgccgccgcgtgtggggtgggggccatctgggtccaaggtggtctctgttctctagagaaaaaggggcagatggggacagacgccccttcctctacaggcttctaccagtttgtgcggccccaccataagcagcagtttgaggaggtctgtatccagctgacaggacgaggctgtggctatcggcctgagcacagcattccccgaaggcagcgggcacagccggtcctggaccccactggtaaatggggccccaggtgggaccctcagactcctgcgtggaaggcagtgtgggccagagtcctgggctgcttggggtgggcatcctcgggccctgcttggccccgcctctctgttcccctatgggagtgatgggggcctccacctccaccaccagcaccagcagcaccacctccacctccacctccacctccacctccaccaccacctccacctccaccaccacctcctccaccaccaccacctccaccaccaccaccaccaccaccacctccacctccaccacctccacctccaccaccaccacctccacctccaccaccaccacctccacctccaccaccacctccaccaccaccacctccacctccaccaccacctccaccaccaccaccaccaccaccaccaccaccaccaccacctccaccaccaccacctgcaccaccacctccacctccaccaccaccaccacctccacctccaccagcagcagcatcacttgttggggagaccctgtgcaactccatgcacagccctgtccctgccatagccccgacccctaagcacagccctgtccaactgccacacgtcccctgcctcccatgcatggtcctggggggtcaactgcacacgccagggtcctagggtcctagacccctgtcctccctgtttctgcctctgtttggggtggagtccaagtctccagaggcggaagcatctgtgttcgtgtgttaatgaacagcccctacagagttcccctagttcacccaggggggaacctagcctgttgggacgaccccagatcccttctgggcttggtactcactgggatatcctcatgcctgcacccagcctacggctctgagctcctgagtggggctttggcctgcccgccactgttccagcccccatccagcaggctggtgtctcctctgatgcccccagcacccaggcgtgtacctgcctgggttttcccgccctggtctgaggtgggtgaggcctggcctccctagccagccctgcccccccaccccagggaactttccagatgctctcgaccagctttgtggctctacatctcttcatcaggaagaacggcgccggatcccaagctgaccgtgtccacggctgcagcccagcagctggacccccaagagcacctgaaccagggcaggccccacctgtcgcccaggccacccccaacaggtagctgactcctgaaccgtgtgcagcctacgacttggtgggtccctcagtggcttcacgaggctaactcttgagtgtggccggggctgcccctgtggggagccatctcatggtggggactgctcccggttctgcaccccgcagttgtcctgagcagctctccaggagttcctggaggaagggcgggcagggcggtgggactctcagtcctccaccccagcgccactctgagccatgctactcccacaccaggagaccctggcagccaaccacagtgggggtctggagtgcccagagcagggaagcagggccagcacgccgtgagcgcctacctggctgatgcccgcagggccctggggtccgcgggctgtagccaactcttggcagcgctgacagcctataagcaagacgacgacctcgacaaggtgctggctgtgttggccgccctgaccactgcaaagccagaggacttccccctgctgcacagcaagtggccctggcgtggggaacagccggtggggtggggggcaggggacaaaatgggggctgtgccgggtctgattgaagctccccgcagggttcagcatgtttgtgcgtccacaccacaagcagcgcttctcacagacgtgcacagacctgaccggccggccctacccgggcatggagccaccgggaccccaggaggagaggcttgccgtgcctcctgtgcttacccacagggctccccaaccaggtagggcacctgcctggctgctcctggcagcgccccaaccgcacgcagccctgggagtgagcagcaaagccccaggcccccctcagactcaagtctctgtctccaggcccctcacggtccgagaagaccgggaagacccagagcaagatctcgtccttccttagacagaggccagcagggactgtgggggcgggcggtgaggatgcaggtcccagccagtcctcaggacctccccacgggcctgcagcatctgagtggggtgagcctcatgggagagacatcgctgggcagcaggccacgggagctccgggcgggcccctctcagcaggctgtgtgtgccagggctgtggggcagaggacgtggtgcccttccagtgccctgcctgtgacttccagcgctgccaagcctgctggcaacggcaccttcaggttggtgcctggccactacagttcctgctgggtgtagccccaggtgatgggctgagggggaaagggcaggcccttgtcctggtggcaacgcctggcagacgtgtgcagtgggccggttgtctcacaggcctctaggatgtgcccagcctgccacaccgcctccaggaagcagagcgtcatgcaggtcttctggccagagccccagtgagtgcccacggaggcccccagcacacccaacgtggcttgatcacctgcctgtccagctctggtgggccaagaacccacccaacagaataggccagcccatgccagccggcttggcccgctgcaggcctcaggcaggcggggcccatggttggtccctgcggtgggaccggatctgggcctgcctctgagaagccctgagctaccttggggtctggggtgggtttctgggaaagtgcttccccagaacttccctggctcctggcctgtgagtggtgccacaggggcaccccagctgagcccctcaccgggaaggaggagacccccgtgggcacgtgtccacttttaatcaggggacagggctctctaataaagctgctggcagtgcccaggacggtg

SEQ ID NO: 2 - DNA, 37214 bp, Macaca fascicularis
  misc_feature 4525..4624: note = n is a, c, g, or t
  misc_feature 4914..5013: note = n is a, c, g, or t
  misc_feature 9472..9571: note = n is a, c, g, or t
  misc_feature 16613..17049: note = n is a, c, g, or t
  misc_feature 19812..19911: note = n is a, c, g, or t
  misc_feature 35806..36357: note = n is a, c, g, or t
  source 1..37214: mol_type = other DNA
atgcccaagatagtcctgaatggtgtgaccatagacttcccattccagccctacaaatgccaacaggagtacatgaccaaggtcctggaatgcctgcagcaggtagagcacaggctgggaggaaaggactgcaggcaggtggagctggggctgggtgtgcgtccctctcccgacccattccagccaggcccctccagaggcagcctctgtcataaaaaggctggtgttccaggtggggtcagagagaggattgacaagtaaaaacatcgtccttgagagtgacagacttagtgccttgggggcctctgaggagcggctggtattcacaggtcaggaggaagcatttccatcagaggggcagccgggggccagcctcactttgaaggtctttgaacctttggggtgcagggaggtggcagcggcgcaggttgccttctcctgggttccttgaggtgccctcttgtacccggctcacacccctcccctccccgagtttcccgctcaggttcccatctgagagctcgtatgtaggacatcagataggacagcgtaagtgtttggatctggaaacacagaacagtttcctattttgagacttgatgcctaattagtcatcttactatttaggctgaaaaatactgttgcatttcgggtaacgttctgcaaatggtctgctgatggcagctggagttgcttcacgccctttagggcaagagtggaaagtgtcctgcggacttatccatggtcccgtggggctctcgccacctgacagcggcctctgcatctgcgaagagctgcccactggctgctgaggcttgtctcagggtggtttgtgtggccttgcctcttcctggcttccccgtaacccttgcccccaactctgttcagaaggtgaacggcattctggagagccccacgggcacggggaagacgctgtgcctgctgtgtaccacgctggcctggcgagaacacctccgagacaccatctctgcccgcaagattgccgagagggttcaaggggagcttttcccgaatcaggccttgtcgtcctggggcaacgctgctgctgctgctgaagatcccataggtgaccctagttcccaggccttccctgacctcctgtggggatggctggcaagggatggcgctgagggtagggtgggcccatggggacttctgccagcagagctcaaggagaattttgtagctgccacataatttctcgccatcgtgggtataaacctagggttgggcttttttgctgaattagggcgtggcaggtgtacacttcgcccgtttgtgataaacgagtctctggggtgtcagattcttggctgtctgcagggctgagttagccgaatgccacccgcctttaacacgtgagaaccatgacctctgtgcttgcttgtgtctgggcagcttgctacacggatatcccaaagatcatttacgcctccaggacccactcgcaactcacgcaggtcatcaacgagcttcggaacacctcctaccggtgggtcagacgagttcacgcctgtcttggggtcctcgagagaaccagcttgacgtggtgctgggtccacagaccacgctgtgctgtagtggagggggcggtctttccagacgctccccagaagtatgcagtgtgctggtgcccaggggtggggcggggcccgggctgtccctagtgcccattacttgtgaggaggcagctttgcatttgtgtgctgaccttgggctggcgtcctgagctcctttcaggtgctgttgtggcagctgtgtggcaggtcagggccggcccccagtgcagctttgcacatgaagtgggaggaggccctgccgcttgtcagagccctgcagagtcttggtgttctgtcgggttcctgtggaggggccgatggcggggtgctgtggaagctgtcgaatctcgtccctctgtccagttcccccgcttgtctccttgctccctcctactcccgggccacgatcctatgccggggccactctcctgcacccggaccacgctcctacgcccaggccactctcctacgcctggaccacattgcagttatgctgacttcctctggctgccagtgctcctgtgtgtctccatacagctcacgctgcgggaccacgctgtggctgttggaggcagctcctcctccacccatagtgctctctccctccaggcctaaggtgtgtgtgctgggctcccgggagcagctgtgcatccatccagaggtgaagaaacaagagagtaaccacatgcaggtgggctcctggctcccgctccggctcagggttccgtgaggcgagtgctgctgggtatccagagtcccaggctgtgctcctgctgggctggggttcgaagttcactgggggactgcaggcgagggcctggtgggggtggggactggctcgggtcctttcttggccatactccagccgcgcactctgcccttcctcccgcagatccacttgtgccgcaagaaggtggcaagtcgctcctgtcatttctacaacaacgtggaaggtacaggcagctcggtgggaccaggcttgggttggagtgtgtgcagcctctcagggcgtcagcctggttgtgcttgccgggtggggcggccagtgcagccgtgtacctgggcactgtcttctgactcgggaccacccgtgttagtcttctgtgtggaagagctcacccagtggtctgagacagccagccggccggactgcctgtggctggtgcctggggccttggattttgggaaggctccctccatttccggacgagaagggctccctgcacctgactactggtgcatacagcagcggttttgctgaacacctgctttgtcttcggggagcctgaggttttgttgcttgcccagcagcgggtgctcctgtgaccggcccttgtaccatcttgggagggtgtcctggaagccgtgtctggcctcccccgaccttgccgcatgtgtctttgtcctgtgctgacgttgcagagaaaaattacagccccgagcgtgactccaggctgagtcctgtgggtccgacacgggaggccttggggcctcttcaggagacgggatgagtgagtgatgggagcagagccagggcacctcgccctgtgactgcaggtggccacagcctgtgagggccaggggtgcttctccacccacgtggctgcccctcgggtatgtcaggggcttctggggctcttcacgaggtctcagagacagtagcagggtgtgcccccgtcggctgcccttacagtttctgtgacctgagggtggcatctgtgcggttggcatggcctgagcttctgtgggatcagagttccctttgtttcctgcctcagttggggctcaagcctcaggtgacgtggccctggagcacctggaaggcgtcggcggtcctgcgggctgctgtctgcactcgtgtttgctgagtgctcagtatgccaggactgaggaccctgaagctgctgttatatttaggacggttctcccctggcagagacggagccgggtggtcccgcctgacccaccaccaggcgtttctgggccctggagggacagggtgggcggaacatgggcctgcagggagacctccacttactggaggctcgtgctgtgttgctggaggcatcttctgtgttgcttcttgtttgctgtgtcttttgttctggtggcaccaaggacctccagtcaccttgatgtgtggttgtccaggcctttttggtggtcctgagaaggggtctgaggggctctgcctctgccgccaggttccctgatggaagctgcccactcgtcctggtggtgcccacaggacgtgactctggcacaggagggcagatgtgctgttggagattctcagcccgtcgtcgtcatttccaaagtcctcacccgcatttctgggacaggacaggagtgcctgctgggtgtccccggtcccatgcagcgggggtccttgggatagtgtggaacgctgagtgtgggcctgtccggcggtggtcctggacaagggtagcacccctgtggccgccgggcctggtgcctggtgcctagcggggaggccgctgaccgcctcctgtgctgcttccatccgtgtcaggcctctgggtgttgggcccccatctgtctccccccaggcctgtgggatcagtccaagaagactcaatccagccccagcctgtcccccttggcttgggctctcactgcccgacctggcaggaggttgcctagcggtgaacctttgcatcctgtctgccccctggacaggctctgagggggtgtctgcaacacctgtgccagcctgggcatctttttttttttttttttcttttgagacgaaatctcgctctgccgcccaggctggagtgcagtggcgtgatctcagctcactgcaagctccacctcccgggttcacgccattctcctgcctcagcctcccgagtagttgggactacaggtgcccgccaccatgcccggctaattttttgtattttttttctttttttttttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttgagacggagtctcgctatgtcgcccagggtggagtgcagtggccggatctcagctcactgcaagctccgcctcccgggtttttacgccattctcctgcctcagcctcccgagtagccgggactacaggcgcccgccacctcgcccggctagttttttgtattttttagtagagacggggtttcactgtgttagccaggatggtctcgaactcctgacctcgtgatccgcccgtctcggcctcccaaagtgctgggattacaggcttgagccaccgcgcccggccttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttgtatatttttttagtagagatggggtttcaccgtgttagccaggatggtctcgatgatctgaccgcgtgatccgcccacctcggcctcccaaagtgctgggattgcaggcttgagccaccgtgcccggccagcctgggcatctttagagtgggctgcagctcctggaggggtctgagaggaaggaaggcaggtattttgtgaatgaagagacagctagagagctggcacccttcctggcctgcgtcctgtgaggactctggttggggacagcaagcttggggttagcctggggcagagcctctgggaggcccctgccccttgtcccccttccccttgcagctcctgtcctcgccccgccctcagctctctgccagggaaggtttggcaagtgccgctgtgcggcagtgcgtgctgattggctggtcgttgctatggtgccgctcagggatatgcttttcctcccctgccttccctgctatccctgggagcatctggagttgagtcatcgctggtgtgtgtgtgtgtgtgtgagtgtgtgtgtgagtgtgtgagtgtgtgtgtgcgtgtgtgtgtgtgtgtgtgagtgtgtgagtgtgtgtgtgtgagtgtgtgtgtgagtgtgtgtgtgtgtgagtgtgtgtgtgtgtgtgtgtgtgtgtgtgtgtgagtgtgtgtgtgagtgtgtgtgtgtgtgtgtgtgtgtgagtgtgtgtgtgtgtgtgtgtgtcagtgtgtgtgtgtgtgtgtgtgtgtgtgtgagtgtgtgtgtgtgtgtgtgtgtgtgagtgtgtgtgtgagtgtgtgtgtgagtgtgtgtgtgagtgtgtgtgagtgtgtgtgagtgtgtgtgtgagtgtgtgtgagagtgtgtgtgagagtgtgtgtgagtgtgtgtgtgtgagtgtgtgtgtgagtgtgtgtgtgagagtgtgtgtgtgagtgtgtgtgtgtgtgtgagagtgtgtgtgtgtgagtgtgtgtgtgtgagtgtgtgtgtgtgtgtttgtgcccgtgtgcgcctctggcctctgcagctgagtcctggctctcagggggcctggcacctcccggggatgggcacaaagcagccacgatgcagccgggagctggggaaggccccattgccccacacggctgccctaagactctggggtgcttcttggaagaggtccttgcttctgtctgtgtttaagcagctccctaaggagttcttgtggttccaggttgggggcctgtgctgtggaggcgagggaggggcaggacccccaactccctgaagccttttctggcctggtcctctcagccagcataggcagggccctttcccagggctgccatgctgcagaggggagcgggccactatgtagcctaggaaaacctgacgttccttccgctggcagttctgggcctgttgtggttggcagggaagctgagccacggtgctcatcacaggggcacctccaggatttttgggagatgcctccgtgggctggggcgatacgctgaggagctgttcttccctgccctgaggagggctgagtgtagccgtcacccctgtcctgtcttgggctgtctcagggaggatgcatagaaccctcggaaccctgctggcccttgtctgatccaccctcaacctcaggccttctaggggcacaggagggctccgtcgggatcactgcgggtgggggcctcccttggacacctgagaccctcaatgggtgctttgtggcacgttcatgattggggcggggggcacccagccctgcctgccgtgtaggagctgttctgtcctgggcgtccctctgtggttgggacccggagggtgtgtgtttaccccgcctcacacctgcagagaaaggcctggagcaggagttggccagccccatcctggacatcgaggacttggtcaagagcggaagcaagcacaggtgagacccctcagtgaggccacgaccactgtccttccatggcccagctctcctgtgacctgtggaggcccagatatgtttcttcccttttctttgttcctttctaagtcgtgaaactaaccaccgtttagtgtgagaaagttgaagcagctctgaggaacgtagagtaaaaagcgaaaccgcctccctcttccctggccgtcagccaccccaggagccaccgtggagcggggacagagccctgcaggctcgtgtctgtgtggaagcacgcacggttttgtctgcacagactgttctgcagtttgccgttttcactcagcgttgtgggtagcttcgcatgctggtgctggtcgtccggcttcgttcttctgaggacagcaaatatgtcctttgtccacctcttttatagtttcagagattcaagttataataaagcttttcttatattgagggggagagctcctgcccctctttttttttttagacagagtctcgctctgctgtccaggctggagtgcagcgtcgtgatcttggctcactgcagtctcaacctcccaggctcaagtgattctctcagctcagcctcccaagtagctgggactgcaggcatgcaccaccacgcctggttaatttttgtattttttgtacggacaaggtctcactctgttgctcagtccagtcttgaactcctgagctcaagagttccacccgcctcggcctcccaaagttctgggattccaggcgtgagcaccacgcccgacccagctctgctttgttttagtggttctgtttgtgtttgtttctgatgacctctatccaggaatagggttggtttttcttttccatggagtttttaaagagatgatgatttacatggtttaaaactcatgtggactccccgtccatcccagacagcggcttcccacctggaccccatgctcaggattagggctccttccgggtcagaagatgtggcggccccggcctcgggtcaccgtcccctgcacggggtgctcacagcatcttcccctctcctcgttcccagggtgtgcccttactacctgtcccggaacctgaagcagcaagccgacatcgtcttcatgccgtacaattacttgttggatgccaaggtgggggctcagtctgcgtcgctgacaactcctgatgtccaggggggttcctgggcttgggaaaggccgtccgagcctttgctgctgcagggacttagagcagcaggcctgggtgggagagctcacctgtcactgggcaggggctcaacctggccagacacacttgtgagcacccccaggccctgggtcagttgttctgagcaatctgggagtgggcaggctggtgggagtgaggagaggcctccaggctgtggtccacaggccagcgcccactcttgatcctgacagctcaggttctctccttcacgtccgatcacaggaagcaaagcactgagaacacgggaagcccactcactcccctcctcccagccccacactcactcccccccatgcccggccccacactcacttccctcctcgtagcctgtactcacccacatgctgttgtgccccacactttttatttagtgcaggtggcatctcgccgcgggtcagtgacttgtgttcacgtggcgatgacgtggtgacgcttccagatccctccattggtttgctcattctcagggtgtatatttatcaagagcccatcgtgctgggggctgttctaggcatagcaagactggcttcattcatgtggagctttgattctagcggtggggacaggtagacagcagaagagtaagcccgtgagccgatgagactgaagagaagtagagcagagcgaggaggtggagacagagccgagtgggcagggcccaagtgcgatgtcggcaggagatggggaatgctgatgtgtctgaggggagcccgagctggtgcagcagagcaagggaggaggcggggggccgactcctgggcagcgattgtagaaccccacggagagctggtgaggtttgccgtggttgcaggtgactcggtgctttgagccgtggctgcccctgggaaccacctggagagcttctaacccaaccaggcccttccctgggacaattctgtcacagctggtaagctaagtccaacactttcacggaaacacagaagatctaaaacggcacgatgaccgtgaagaacagagctggaggactcacctcactggtttcaagactcctctaaaactgcaggagtggaggtggagacggctcagctcaggcacaagcctgcaggccatggagaaggcagcaagctccagctgacccacacgcttgcggtcattgttttttttttttagttggaatctccctctgtcaccccagttggactgcagtggcaccatctcggctcactgcagcccccacctcctgggttcaagcgattctcccacatcagcctcccgagtcactgggattacaggcgtgcgccaccacgccctgcccgtgatgattgttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttttgagacagagtctcgctgtgtcgcccagggtggagtgcagtggccggatctcagctcactgcaagctctgcctcccgggtttttacgccattctcctgcctcagcctcccgagtggccgggactacaggcacccaccacctcgcccggctagttttttgtattttttagtagagacggggtttcaccgtgttagccaggatggtctcgaactcctgacctcgtgatccgcccgtctcggcctcccaaagtgctgggattacaggcttgagccaccgcgcccggccgatgattgttttttgacaaatgcgccaatgtaattgagagaggaaatgaaggttgatttttggttttctgaaaaagtggtgctaagaacagctggatatctgtttggaaaacagcgaatcttaactcttgctttactctatataaacctaaatgtaaaagctaaactaaaggttatagaaagaaacatgggggggtctttgcaactctggggtaggcagagattccttagtgtggatacataaggcactagccgtgaagaaaaacataaaatttggatttcaccaaaatttaaagcttcaacacttgagaagacttgagaaaattaaagagcagttatggaaagggagaaaatgcttctgtctttcagaggacttaaaaaacatttttcagccctcctcatattttaaaggacctttgaccagagtatgtaaaattctctcataactaagcaaaaaacccatttaaccagtgggaaaggatctgcacagacgtttcaccaagatggacggagtggccagttgggcacatggaaggacgcccagcatctccagtcacaggagaaggcagattaaagccacggggagccggcactacggtcccgctagcatgctgaaattcagaagcctgagtgtggcacgaggaggtggaacagctggatctcatccattgctcctaagctgtgaagctgtgtagccactccacaaacacgtgtggcgaacagccgaaccgggagaagggaagacgtgttcaaagatttgtgtgtggccaggctcagtggctcacacctgtaatcccagaactttaggggccaaggctcagggatcgcttacgcccaggagtctgagaccagcctgggcaacatagggagaccccatctcaaaaaaaaagaaaaaagaaaaaaaaagacttaaatgtgcaagtttaccagagctttgtttaagttgccagaactgggaagccgcccacgtgagtccatccactggtgaacggacaaaccgtggttcgcaaaccccaacagcagccgcgggcatgggctgtggtcacacagcagcagggagccagtgagtctcagatgtgctaacccagaggccaactgaggaggacctactgttttttgttttttgatttttgttttgagacagagtcttgctctgtggtgcaggctggagtgcagtggtgtggtctcggctcactgcagcctctgcctctcgggttcaagcagttctcctgcctcagccttccgagtatctgggactacaggcacccgccaccacacccggctaatttttgtatttttagtagagacggcatttcgccatgttggccaggctggtttcaaactcctgaccttgtgatccactcaccttggcctcccaaagtgctgggattacaggcgtgagccaccgcacccagctggacctactgttttattccgtttatatgacgctgtattaatagaagaggcaggggtagagctggtggttatgtggtgcacttaactgccagaactcagtacccttaaaatgggcatcttaatgtgtgaaaacttaaaagtaccaaagctggaccccaccccagcttgctcccatgacactctgtggtgggacctgggagttggggtttttttttttttttttcagatggagtctcactctgttgcccaggctggagtgcagtggtgcaatcttggctcactgcaacctctgcctcccagattaaagcgattctcctgcctcagccttctgagtagctggggttacaggcgcacaccaccacgcccggctaatttttgtgtttttagtagagatggggtttcaccatgttggtcaggctggtctcgaactcctgacctcatgatccacccgcctcggcctcccacagtgctgggattacaggtgggagccaccgcgcccggcctggagttgggtttgtaaatctccctgagtggggccggggcagagaactgctgggtctgggtcttcctggctcctgtggtctgtggctggctgactgcggtggccgggggctcccaggacgtcgtggccgtccgtcttgctgagcgtggcgcgtgcctttccatgctgtgcgtgagcgtctcccggcgtggcgagctgctgcttcgggtggggcggtgttgccaggttgccatccagatctggcctctcctctccacgtcgccggcggtgtttgtcatcggcacttgcgatgttcgatgccggccacatgtgccttggtttccctccttcctgcccctgtgagctccagcaccgtgtcccttctcttcctcctgtagagccgcagagcacacagcattgacctgaaggggacggtcgtgatctttgacgaagctcacaatgtggtgagtctccactgcctcctaaacgcctcctgtttcttcaagagcagcgcaaacctttctggaggggctctggccaaacccttgcagccttcggtgcccaggactgaggactgagcaccccaggagcttctgcaccccttcccactctgatccgatgcctctgctggggctggagactggccagctgggccagggacctgcccctcaggcgcagggcccccacaggccgctccccaggccttccctctagccagctaggggtcagcctgggccaggggtgtctcctctgccctcggcttctgtcaccagggcagcagcaggcgtgtggtctcgcctgcagtgtctctgcccttccggccacatggcctgaggctgaggcagaattgcttgaaccctggaggtggaggctgcggtgagccaggatcacaccgctgcactccagcctgggtgacagagcgggattctgtgtcaaaaaaaaaaaaaatgttgactgggcgcagtaggtcatgcctataatcccagcactttgggaggccgaggcgggcggatcatgaagtcaagagatcaagactatcctggccaacatagtgaaaccccatttctactaaaaatacaaaaatattagctgggcgtggtggtgggtgcctatagtcccagctactcaggaggctgaggcaggagaattgctcgaacccgggaggtggaggttgtagtgagccaagatcacactattgcactctagcctggtgacagcgagactccgtctcaaaaaaaaaaaaaaaaaaaaaattggcaactccattgaaataattgtttgtttgtgttttgagacagggtctcattctgtcgtccaggctggagttcagtggtgtgatcttggtccactgcaacctccacctcctgggctcaagccatcctcctacttcatcctcccaagtaaccaggactacaggtgcacaccaccacgcctggctaatttttgtattttttgtagagatggggttttgcatcattgcccaggctggttttgaactcctgagctcaagtgatctgtccacctcagcctcccaaagtgctgggatcacaggtgtgagccgctgcgcctggcctgaaataatcgtttctcaatattggtgtgggccagacagtcgtgtttggacctgcttgtggcctggccttagagaccccagggcgtggctttggggacttggctgtcagcctcctgtgccttctgcacccaaccccattcctgctttctggaacccccgatcctatcctgctctgtggtgatccgggtgatttgggctcccgatcctatcctgctctgtggtgatccgtgtgattcgggctcctgatcctatcctgctcggtggtgatcccggtgattcgggcccccgatcctatcctgctctgtggtgatccgggtgattcgggctcctgatcctatcccgctctgtggtgatctgggtgattcaggctcccgatcctgtcctgctctgtggtgatccgggtgattcaggctcctgatcctatcctgctctgtggtgatcccggtgattcgggcccccaatcctatcctgctctgtggtgatcccggtgattccggctcccgatcctgtcctgctgtgtggtgatcccggtgattcgggcccccaatcctatcccgctctgtggtggtccgggtgatttgggctcctgatcctgtcctgctctgtggtgatccgtgtgattcaggctcctgatcctgtcctgctcggtggtgatcccagtgattcgggcccccgatcctatcctgctctgtggtgatccgtgtgattcgggctcctgatcctatcctgctctgtggcgatcccggtgatttgggctcccgatcctatcctgctctgtggtgatctgggtgtgctcggactccaggagaagatgtgtgaagaatcagcgtcctttgacctgaccccccatgacctggcttcaggactggacgtcatagaccaggtgctggaggagcagaccaagactgtgcagctgggcgagccccacccggagttcagcacagactcccccagcccaggtgcgttcatagccaggctgctcggtcctgaggcctgcgctgctgcagggggcagcctgccctgtggctgtgtgtggtcggcctgggcactgtctgttcaggctggcactgcagggcacccacttctctcagaggcttctcgggtgtgaattgtttggggtcctgtaggctgtggaagggctggtatcgttcagtagttctggtattttccgaagacctatgtcttctcccagccagtgtcaacttggcctctactgtgtaaaattagagaactatactttgtgaagctgagatgggagcatcgcttgaggccaggagttcgagaccagcctgggcaacatagcgaaacctcgcctctgccaaaaaattagccaggtgtggtggtatggtcctgtggtcctgtggtcagagcttttctggaggctgaagtgggaggagctcttgagcccgggaggcagagcttgccccagatggctccactgtactccagcctgggtgacagagtgaggctgtctccaaaaaaaaaaaaaaaaaaagtaaaactaactatattcactgtaagggcattttgcgtgtttaaatgacccacacatcgggcctgcatcacttgctgtgcccgtagattccttcccagtgttcgtccagaggcgtatttccacacagcgctagtcacggcgtacgtggaaaacgcggaaacccccatggatgtggtcggttggtctgtactgtctgtgttttcttcctttctttttttttttgagatggagttccactcttgttgcccaggctggagtgcaatggtgcgatcttggctcaccgcaacttcctcctcccaggttcaagtgattctcctgcttcagcctcccaagtacctgggactacaggcgcgcgccaccacgcccagctaattttgtatttttagcagagtcagggtttctccatgttggccaggctggtctcaaactccaggtgatccgcccacctcggcctcctgaagtgctgggatccaggcgtgagccgccgcgcccggcctgtgtcgtgttttctgcgtggcctctgtaaacagctgactaggagtctgtgagcatcttcattggttccgctgtgacttgcgtgtgagcatctccagcatcagctttcctcattttcctttaaaatcgcacatgtggatggacaccatggggaccctggaccctgggcagcctcgtcctcacctttctcaccagggtggctgcttgggagagagtgagtttgcgaacttggcatttgttctggctgtaggatccgtttatatatatacatatatttaagtgacagggtctgactctgttgcccaggctggaatgcggtggcgtgatcttagctccctgtagcctcagcctcctgggctcgtggggtcttcccgtctcagcctcttaaagcaccgggaccgcaggcggtcgccactgccatcggatctcaaggctgccacgcagggcgctgcagtaggggaaggggttcctgggactttgaacgctccacctccctcttctccacagtcccccaaccccacctctaactgggagtggatggccacctctttccatcctttgcctggcgcagggtggggagaatgacaggtctccttcccgcatctcggcagctgccctttcgtcggttatatgacgtgggagagaccccggccacccccagggctgtgtgacgtgggagagaccccggccacccccagggctgtgaacaccgccacaggggccttctccactcttcagttttgttagttacgtgacgtgcagggcttttcgttgtaactagtgggggacgtgtggtgaggggggcttctgccgtctcattcaggaccggaacttcagttttcatcgctatttgtcctcctacgcctttggagatggggtctcgctctgttacccaggctggagagcagtggtgccatcagggctcacggcagcctcctcctcctgcagcctccgcctcttgggctcacacaatcctcctgcctcggcctcccaaactcctgggacgactacagacgtgcgccaccgtgcttggcagggttcattcttttcctcacactttatttattggagagcccggtagagcgcccggtttgcacgatctggttgttgagacgttgagaggaaccctgcggagtctgaatctgggtatgggaggggcagtcacacgagttccccggtgtcctcgtctcctccgcacttaggtgcccttaggtggcttgaggaccccagttagactggggccaccctttgcctggctccaggcgggatgtcctttctttttttttttttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctgggactacaggcgcccgccacctcgcccggctagttttttgtattttttagtagagacggggtttcaccgtgtcagccaggatggtctcgatctcctgacctcgtgatccgcccgcctcggcctcccaaagtgctgggattacaggcttgagccaccgcgcccggccgggatgtcctttctatcaggaggctcacagtatctgctttccagtttcttctgatgttaatgttgttgctgctgaacacctgactctgttgattattgggggattgcaagatggtagtgtcccaattctgtcgcttagtcagttagcaggaataatctagcaccatgctctgcatcgcatcagccgtgtggtgatcccattcacacaggaaaggcgggataaatgctggctgtgggccgggtgtgctgtcccgcatctgtaatcccagcactttgggaggccagggcggtcggatcacgaggtcagagattgagaccatcctggccaacatggtgaaaccccgtctctactaaaaatacaaaaaaaaacccaaaacatgagtgggctgtggtggcgcgtgcttgtaatcccagctaccggggagactgaggcaggagaatcacttgaacccggggggcggaggttgcagagagctgagatcgcgccactgcactccagcctcacaacaaagcgagactctgtctcagtaatcaatcagtcaatcaatcagacgtcatcagtgaatgtacgtgagcgtgtagggattggtcctgctccgcgcgccaccctcaatctcacttctgacctccgttctccacctcgaggtgttttcatgtgggcccgtgtgtcctgaccccagcagttgtggcctctttgctgtctgggagtcaggatttttcacactcatgtcctgctccagacctggaatcagccaagtctccaagaagctctggtttcttttcctgcaaggtggtatttcaggacccacgatgcggcaacaggctggtcatggttactgggttggtcattgttactgggtgtttttgtggagatacatccatacgcacagatgtgttcacaaatgttaactctgaaggtaaaccacccggctgggcacaagggctcaccggtaatcccagcactttgggagaccgagacagttggatgacctgaggtcgggagtttaagaccagcctgggcaacatggtgaaaccccatctctactaaaaacgcaaaaattagccaggtgtggtggcacacgccggtagtcccagctagttgggagacagacatgctggaacctgggacacggaggttgcagagagcagagatagtgccgctgcaccccagcctgggtgatagggtgacaccctgtctccaaaataaatagataaataaagggaaaacgcctcctcttggtgtctccagtttggactggctggcatgggttccggtagcctcttcccccatggtggcgtccttttttccatgccgggaatcctggttctcaagggcggggttgttggtctgagtgtgacacagactgtgcctttgctgcctttctcttgcccagggctgaacatggagctggaagacattgcaaagctgaagagtaagtgctgccctccccaccttcttgcggctgggtggggcctccccctgatgcctcctcaacccgacctgcagtgatcctgctccgcctggagggggccatcgatgccattgagctgcctggagacgacagcggtgtcaccaagccagggaggtgagaggcggggagccagctgcttcactggaggcccagcctggagctagagataggccatggtgcagtcctaggctgtcccaccagtgaggcctgttttcaggcccgttttccctttttgagacctggtaggagcacctggtttgcatgatctggttgttgagacgttgagaggaagagcacacgcatccacggaacagcacacgcatccacggaacagcacacgcatccacggaacagcacacactcccacggaacagcacacgcatccacggaacagcacccactcccacggaacagcacacacacccacggaacagcacatgcttccacggaacagcacatgcttccacggaacagcacacccccacggaacggcacccacggaacagcacgcacgcccccacagaacagcacacacccccacagaacagcacccacgcccccacggaacagcacacacacccacggaacagcacacacgcccccacagaacagcacacacccccacggaacagcacacgcacccatggaacagcacccacggaacagcacacactcccacggaacagcacacgcccccacggaacagcacacacacccacggaacagcacacactcccacggaacagtacacgcatccacggaacagcacacacgcccacggaacagcacacgcatccacggaacagcacacactcccatggaacagcacacgcatccacagaacagcacacacactcccacggaacagcacacaacagcacacactcccacggaacagcacacgcccccacggaacagcacacacacccacggaacagcacacactcccacggaacagcacacgcccccacggaacagcacacacacccacggaacagcacacacgcccccacagaacagcacacacccccacggaacagcacacgcacccatggaacagcacccacggaacagcacacactcccacggaacagcacacgcacccacggaacagcacacacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcacccacggaacagcacgcacgcccccacagaacagcacacacccccacagaacagcacccacgcccccacggaacagcacacacacccacggaacagcacacacgcccccacagaacagcacacacccccacggaacagcacacgcacccatggaacagcacccacggaacagcacacactcccacggaacagcacacgcccccacggaacagcacacactcccacggaacagcacacgcccccacggaacagcacacacacccacggaacagcacacactcccacggaacagtacacgcatccacggaacagcacacacgcccacggaacagcacacgcatccacggaacagcacacactcccatggaacagcacacgcatccacagaacagcacacacactcccacggaacagcacacactcccatggaacagcacacacacacccacggaacagcacacacgcccacggaacagcacacactcccacggaacagcacacacatccacggaacagcacccacggaacagcacccacgcccgcatggaacagcacccacggaacaacacacacccccacggaacagcacacacccccacggaacaacacacactcccacggaacaacacacacccccacggaacaacacacacccccacggaacagcacacacacccacggaacaacacacactcccacggaacaacacacacccccacggaacaacacacacccccacggaacagcacacacacccccacggaacaacacacacccccacggaacagcacacactcccatggaacagcacacacacccacggaacagcacacacacacccacggaacagcacacacacgcccacggaacagcacacacccccacggaacagcacacacccccacggaacagcacacacccccacggaacagcacacacacgcccacggaacagcacacacgcccatgcggggccgctgggtttcctgcagtttgtcctcctccaggcctttccctggacctcctcaggtgcctgttagaatgagaccttcttgttaggacgatgagtgtcccagccaccacctcttttggactccgggaggcccggaacattctgaaggctccgtgggtctcctgtcttctccgcggccagggcagcagcatttgttgtctgtcctgcaggcagatgagggtcaggcctggggcccgtgtggggctccactgagtgcacagccgccaaggcgagcgcgttctgcctgagcgctgaccttgtggcgactcccagttgcttccaggtggagccagtgtcctcagcgtaatgctcaaggctctggcgtggctcctcggccacctgcacccccaggggcccctcctgtagcttctgctgccgccatcactgtcactctcccaaggctttggggactctgcccagagccgctgcctcccagaagcccctgacgaccccccggcgaccccatccctccctctgatgacggcctctgccccgaggcggcttttccttggtgctgttcttgtgccggccaggcctccacttcccatctggaggctcctgagggcggaggcctctcacccccaatgccggcgtcccctggagggctcagtttgtttccgagggaaggaaacttccacagttgctgccttcagttcccaaagctgcagcctgatcccccgtccaggctcgagcctgccttcttctcctcgcagctacatctttgagctgtttgctgaagcccggatcacgtttcagaccaagggctgcatcctggactcgctggaccagatcatccagcacctggccggacgtgagtgccggggcggggtccttggtgtgggcagacgtggcgtagggggtgcagcaggcctccatcctggcagtcagggctcgcctggccatcacctggccgtcagcaggaacaggctcacagaacctcacctggtcggggcgtggaggcgttagtgccggttgcccactgccgtagagcctgtcccagttctgcagctggcggcttcgtcctgcagactcatcccattattccccttttaaggaagagcagcccaaggccctggctgggttgtggggcctcaggcttctccacacaccctgagttctgcttctcagagctgtggggtccagaggctttgcccagaggcagtgtcgtcatgggctgctctggtttgagatgccggcccgccggggcctctgctgtgctcccaggtgctggggagggcggctcttgctgcttcaaaccttaggtgaccatagagcctcttttcaagttccaccggcccccttggagacgctgtccctgcctggctgctctcctggctgctgggaagggcaggcgagctgcccgccctgagtggacactgcgctccaccctgggccccccgttgggcaggagatggagcttggcagtcgggctgagctggcttatggctggaagggcgggggctggggtcggggcctcccctgcctgcagtgcgggtgtcagcgcccggcatgctacctccccaggtgctggagtgttcaccaacacggccggactgcagaagctggcggacatcatccaggtggggcctgctcctctggcgtctccttccctgatggaagccgggcgggtgccttctcctgctgtattagttactgactctagacgtgggggtgggagaacggcccccacaccacctgtttccgactggcaaacgcttggctcctttcctgtgacccaaacccacactgggcctcccgcggggatgggggaggacgaggtctggtggcccgtgcccagggtgacgctggtgggggaggacgtgaaggggagtgggggccagggagccgctcctgccctgtccgggccctcaggccggggggacccactcctggcagccccagctacacgcagatgaagagctctggacacacgtagcttcctgaacagcttctccagggacagacaaacggggaccctgcaggttgccggcgggggtgtccctgcgagcccatgatgggggatgcggccccggcccccttcttggccccgtcctgccctgtgatgcccgtcccctcccatgtgaggttgctcctggctctgtgcctgtggcagcacctgctttccctgaggcagacgggcttgtgcggtttgtggagggcaagccccatttttgggtgctctgggcacacacttgcctctcctggacctcggtgcattttttagaagcagacccacaagtccctgactgggaggccatcagtccttggggaaaccgaggcacaggtggctgtgtggagccatgtgaggctgcctctgtgctgcccacactgaacctcctggccacacgaggtcatggcagggttaggtggggctgcccagctctgtgacaacttcgtgagtgtccgcctggggtggggggtgcttgagctgggggaggccgcagaagaaccctggaatgtaccgccggcctgtgcatgcagtgcggtgctctgtgcatgctactgggcgtggagctgcaggccccaggacgaccaggcactgccttcttcatagacgtggctctgagcaggggcgagacctgggcagggaccaggtggggtgggcacagcaggtggtagggcccaggctcagccctacctccaccgtggccttctctgcggccagtgatgccacagccagtgtcttctctgtgtggcagctggggctggaaagacggcactgctatgtctcccagctcctccccaaaagcacggtgggcatcccagggccagacccctctctctgtggctcctgcctgccaagggctgctttgccgtcccgcctggagcgtggttggctcttcaagcaggaggccgtgcacctgccaggcggacctgcttccatgtcctgacgggtcactcaaagcacctccagcacatggccatcgaggcagccctgcagcccagggcctggagggcaggtgtgagctggcccgggcctgcccctccctggaatgtggcttcccaggctccagtatggagcagtgtccacactctggaaccaaatcttgttcaccccattggtttggaatttcctttggctgctgctctaagtagccactgggagatgactcagctcctcccaaccctggggactgcacaccgtgttcaccctcggctgctcctgagtccctgcaggtgtgggcactgctcatagggctgctgctgggctggacttgcagccctgaggcacaggtgaccctggggacagagtggggctagagtggggctagtggccccaggaagaggatgtgggatgcacggttcagttggaggcaagtccgggcagggccaggctgtgtgctggggaggtagagcttagctggaggcaaactctgggcagtgctggcccgtgctgggcagccagtcaccgctgggcccctgtgagactagtgggtcccctttcttctctgcctgagctgccctggggctgaagcacggggcggtttgtagtggtgtttttttgagacagggtctcgctctgtcaccccagcgggagtgcagggccttgtcaccagctcactgcagcctcagcctcctgggcccaggccatcctcttgcctcagccacctgagtagctgggaacacaggcgtgcaccaccacgcccggctaattttaaaagcttttttgtggagatggggtcttggcgtgcaccaccacgctcggctaattttttaaatttttatgtggagatgggtcttgccatgttgcccaggctggtctcaaagtcctgggctccggcagtctgcccaccttggcctcccacagtgctgggattacaggtaagagcttccaggcctgcccatcagagggctggctttttgagggaagctgtttcctgaggcagcctcagcccctgctcatggcagcccctggtgtcatggtgaatttgttctgttgtttccatccctgtgggccaaatgctttggtaaaacgcaaggccttgtatttagctgtcttgacagtgaaaatggctgggaaggaaggaaggaaggaatggaaggaagtttctctctcccgtgcatacccaggcacgtgcacacgtgcgcagtccacgcacgctgccggtctctgtcccagccgcggtgccggcccccgtgttctccagttcagcttctccgcatttctgtgaacgattccggcttcttgatgttcccagcagaactccctcaagccttcggcttctccagggctctgacggcggctgggctgacgtggccgctctgcggagcctgttggggcttcacgttcctgttctggccatttccggctctttttccgggaagggtcatcacgcgctcttttcctagtgcaggcatcgtccctttcccgttgatttgcagggttctctgtaagatctgaggaacacgtgccaacatattctttcctggtctcgtgctggtcattttttccattttcacgacaggtttggtgaacagatgtttccttgtcatcagacttttgtcttgagcttgtatcccccgaccaagatgctaaactgggtcttgtgtgttctccaaactgcactgtagggtggcggagctttgtgtcggggcctccgcaccttccggcgtcacctgtgggggtgtgaaaggcagactctacctggatttttcccagcacgccacaccggtggttccgtctgctgaccgagtggctcggcttctcccaactccgctgggcacctgccacactgttttcctcacggttttgttcgtggtggttttgtcgtaagtcctggtgtcggccagaaccaatttctgtttttgtttgtttttgagatggagtttcgctcttgtcgcccagggtggagtgcagtggtacgatcttggctcactgtaacctccgcctcctgggttcaaatgattctcctgcctcagcctcctgagtagctgggattacaggtgcttgccaccacgcccagctaattttcatatttttagtagagacggggttttgccgtgtgggccaggctggtctcgaacccctgacctcaggtgatcctctcgcctcagcctcccagagtgctgggattacaggtgtgagccgccgcgcccggcctcttgttcttcttaaacctgccctgatgttttctccatgatgcatgttggagtcaacctgtctgcttcctttaaaaatcttgctgtgattttgactggaatgtgtggaattcctgtgtttttgagtcagtgtctctctgttgcccagtctggagtgcagtgggaccatcacagctcactgcagcctcaacctcctgggctcaggggatcctctcagcccagcctcccaagtagctgggaccacggcacgtgccaccacgcccggctagttttcatatttttttggtggagaccccagggttgcaccatgttgcccaggctggtctcgaactcctgggttcgggcagttcgcccctctcggcctctcagagtgctgggattacaggcgtgagccactgcactaggccatgttgaatttctagattaatttgggacccttaggggcacagagaggagggctgggccagctggtgggaggagaggcctctcgggctgccgcatttccagtgcacggagatggcctgcgtagggggaacagagctcactgggggtccctccagggaggagaaagggtcaggcaggtgccagctcctgtccatcagcctggggctgcatgatggcaggggctggtgaaccgatgacctctgggtgtcctgtgaccttccgtgtgtggggctgatgccgcagaagatcaggtggcctccagggctccgggctctggcctcaggctcctcacgggactgcacctcctctctttcagattgtgtttagtgtggacccctccgagggcagccgtggttccccggcagggctgggacccttacagtcctataaggtaggggccacctccaggaggcaggtggggctgggggtcctgcagtcctgaaaggtaagcggctgcccccaggacacgggcggggtctccacacttctggtcctgtcccctccaggtgcacgtccatcctgatgctggtcaccggaggacggctcagcggtctgatgcctggagcaccactgcagccagaaagcgaggtacagacctgggcccacactctccccgcccgcccgggtgtggtgctcagcaccagcatgccacaggctaggcacgtggccagccgtgggtctcctgcccccctgggcctggccaccttctccatgtccaggccaatccagagcgttctcctcactgtccctctgaaggctggagttactgagagacatgggagagggcctgatggcaccgtgacctgcccagagtcacctggttggtggcggcagagccacagctcagccaggccttcctgctgggacacactggtttatgccaaggccgtcagcacggagccccacagtgaggcacagctttccctgctgcctccacccagcgcctggctgggccaagcctcagggtctcatctgaagggggcccgactggccctgttgtccgaagcctctggtgcgctcggccctgaggccccacgtgccttgttggcttcctgtgctctgtggcgtcttcgagtcggtgctgccgggtacctgtgtggatggggcccgtgagtgggccctcggctccgtgtccagagtcctgtggttcttggagtgtgtccggccccacccggccatgtggtgtccaaggggggctttgtggcggcagcggtgggagctgctgccccttctcgtgctccagtggggccttgcctctgggcttggttcgttcctctctggaacgttctttctcagagctctctggcccgcggtggcacgacgtggccctggctgaagcagcccccgcgtggttgctgtggttggtctgcctggccgagccggaaggggaggcccgggagggcgtcagggttgcgtggcttgacccccggtcggtggctcggtgatggcctcgtggcaaggcgtctcccagcaggaagcgtccatcctggggggtcgtgggaggcggcgcgtgcctgaggcagctggcgcacagatcctgagggcctggaatctatttgttccattgacctgtgacgtcacttgacttctgagaagcagccgctccctgcgttgggtgtttgtaggaaatgagttcctggaggcggaggtcgggaagttcccccgctgcagggcacgctcagccccaggaagcggggtgacctttgctgggagaggtgccttgtccctgctgaccccgagtcacagtcagcgtcgttctgcttgtgccgtgtttaattcctggtgtccggtgcccaggctcagtctcctccccaggccgcggccgctgcggtcagtcagtcaagcacttctaggaacttcctttgaaccggtgcccttgttcgctgctgacgccactcggaggcttcctggcggagcggggaactcgcacttcctcttgtgcgcgtccctggaatgtcgggacgctggcgcttgcttcccgtgcgctcttggcgggatgagcttgccctgagctgcatgtcactgtttctgcagaagtttagggttggagttggctgacctctctggaggtgtccccagcatctgcctggggtctgcctcgtactcccgggacccccagtcccccagaggggaccccagctggcagactgcgctcacactcagggcagcctcctttgttctgacttctgcacgctgggcctgggtggctgtgggcggctcacttgcttgatgccagtgggtgagagggtgatgggcagagaggcaggcggtcaggcccccagtcccgtcctcacaccctgtgtcccctgctcccctccgccccacagggaaggtgttgagctactggtgcttcagtcccggccacagcatgcgcgagctggtccgccagggcgtccgctccctcatccttaccagcggcacgctggccccggtgtcctcctttgccctggagatgcagatgtacgggccacccctgccaggggcctaagcactggtgacgcctgtgacgttagtggggtgggagtgctgggggtccccatgagccggggtgctgggggtcttgggcctcactgggggtctcgggccttgagggctaaaggggtgctggtgtccttccccactgtctgtccctccggccacgctcagccctttcccggtctgcctggagaacccacacatcatcgacaagcaccagatctgggtgggggtcgtccccagaggtcccgacggagcccagctgagctccgcgtttgacagacggtgagggcctgtccctgggccgtgctggggtgcgaggtcggggagtactgagccctgaggccctgagcagtggcctctcggctctgtgcaggttttctgaggagtgcttgtcctccctggggaaggctctgggtaagtgcccggcacaccccagctgtgcccatcctggatcctggaccctgctcccaagagctggtagggaccccggaagacgccccgcccctgccctgaccccagcccctgcacttccaggcaacatcgcccgcgtggtgccctacgggctgctgatcttcttcccttcctatcctgtcatggagaagagcctggagttctggcgagtatgtctcccctgtgtgtcctgggtggggtgggtgagggcagggctggagcacgaagcaggcagtggccacagctcctgcctgccctcatcggatcggcggcgtaaccagggctgccgtgtccctgcctcttcctcccacaggcccgcgacttggccaggaagatggaggcactgaagccgctgtttgtggagcccaggagcaaaggcagcttctccgaggtcagcacttggccggggctctgggtctgctgccccctcgtgcctcccgtgcccctcacagcttccccgaggctgaccaccagccctgaccacgggctctggcggctcccgcctgcctcttcagggcccctgcgtttccttcctggccctgagtgttgcctcttatcttacaaagcccccagcactgggtgggtgtggcgacagtggtcctcctgcctgaggagctatagccggcggccaccctggccctgggattccccgtgttttctgggaagcactgagcaggcgcaggatcagcctgggatccgtgccaggaagaagtttccagaacccagttggccttcctggctaggatagtccttcatcttggaggatgagacctgggtctccctcatgggggaagaaggggccgaggtgggctccaggctgagcctgaccaactttccttccagaccatcagtgcttactatgcaagggtcgctgcccctgggtccaccggcgccaccttcctggcggtgtgccggggcaaggtgagctctctggggccctctgtcctgacccggttgcctgttccccagtgggtgctcatggctcctcagctgactttgggccctgggggctgcctggtcccctccttgggccccacaagagcaaagcactggtggcctgctgggagcgtgccccgctctgggtcgggcaggcaggatgcaggaggttcctggccacgagagttggaggtggcgtctgggagctgtggaccccaagtggggtcctgacccatagatggggcttcctcccacccctggttggagcccccatggccccaggcagctccctggtgtgtcccccaggccagcgaggggttggacttctcagacacgaatggccgtggtgtgatcgtcacgggcctcccgtaccccccccgcatggacccccgggttgtcctcaagatgcagttcctggacgagatgaagggccagggtggggctgggggccaggtgagttacagcagggcggggctggggtaaggcaatctggcgactgagcccccctgtgaccacgggagcccctgtgaccgagcctcctcgccctccagttcctctctgggcaggagtggtaccggcagcaggcgtccagggcggtgaaccaggccatcgggcgagtgatccggcaccgccaggactacggggctgtcttcctctgtgaccacaggtgcgtgcagtccgggggcaggcgtggcgccaggggacacggccacatcccactgggccctggactccttccccacatgaggccccgtctcccccagagcctctctggcgactcggggtcagcctgaggcccctgcggcagatgagggtcttcacttgggtgaactgaccccctgaagcggctgtgggcagggcagcagggctgtggccaccccccaggttcgcctttgctgatgccagagcccagctgccctcctgggtgcgtcctcacgtcagggtgtacgacaacttcggccacgtcatccgagacgtggcccagttcttccgtgttgccgagcgaactgtgagtttctgcccagggaggggatgagggtgttgtccccagaggagccagaaatgggcccacccacccccagtggttctgcagatgccagcaccggccccccgggctgcagcacccagtttgcgtgaaggagaagatgctgtcagggaggccaagtcgcctgaccccctcctctccaccaggaaagctaagagtctggacctgcatgtccccagcctgaagcagaggtcctcaggtgcagatggccagtgcgggacgggcagtgtggggcgggcggggcaggcggattggggcgggccgtgtggggcaagcagcggggcaggtggtgtggggcgggcggcaccaggcgcccagggtggaggcggctcacctggctttatgcactgcccctcccacctccaaaggctgtgtctgcctcctagggtggggttcccacgggctggaaccctcccctacaggcagagaaggcctcaggcaaagacgcgccccaaggctgggactccccctaatagcagggaggacacccacaggcaggggcccccaaatgctgggaccctccctcaggaggggctccgtcataggcagggacccccagctggggccccgcggtgggcttcactgcgcactcgggtactctgcaggatcaccagctgccggggaccccgagagtagcctgtgtgtggagtatgagcaggagccagttcctgcccggcagaggcccagggggctgctggccgccctggagcacagtgaacagcgggccggggaccttggcgaggagcaggtgcagttccagggccttgggatggacacagaccctctgtctcctgaggccaaccccaccccgcccatctgacttcaggcacctccccacacacccctgtaaatcccctgcctgtggggcgggcaggcaggtgagggatcccagctgcctggctgtgtgtgtgccctccacctcgcctcacccacaggctgctggctcccaggtggtgcatgccctggccctcagcgggtgcccccacatcactttgattctctggcaggtcagcttggctcagggcactcaaggtcgggtgcccctgcccctggctgtgcctgaggctggcctttctccaggaatgtgctgcggttgggacccaggttccttcctccttggggccttttgccccagaagcccataatttctcagctttctccctgcttcctgctgggggccgttctcttgctgcctgtccctgcccttcaggctcctggagtgagccccgggtgcaggcaccagacacctgtgtccccttcctgccagcccctcgctgtggtcggactgtccttcctggacctgctcttacgtgtcaccacctgtgagcctcatgagccgctggcgtgacttggacaggaccaagttgtgggactgtcaccagggtgtgccgcgcccctcccccgaccttcgtcttggctactgctcagggctccttggggccatcttccctgtgggcccaggtgctttggggccctggagtgtgtggttggaaccacagctgtgtcctcctcaggcccacagctgctccaccctgtccctcctggctgagaagaggccggcagaggagctgcgaggagggaggaagaagatccggctggtcagccacctggtgcgtgagctgtccctgcacctgtgccgaccaccatagacacgtgtgggaacccggccctgggtgcccccagccaagttgggatgtgggcccgatgggaccagggaagttacccccgggagctgatgtccagggcggctgtgaagctgtcggcccaagggctcaagggcagctgggatcaggccccgcccttgggcaggcttggtttgggggctgcagcactgggtttggccctggcatttccctcaagtgtggacgtgcacctgcctcattcgaggggcgcagcccattcccagccttggatgaaagaaccgagttatagtcggagccaggaagccccctgcctgctggaaagccccaagtgtggcagcctttgtccatgtcccttggcttctgggaagaactgggtggtgcccaggcagggctggtgccatcaggaagtgggcggctgccgaggggcctgggctgttgagggcctgggtggggagtgccggggctgcccctgccttggtttccacgtttccgtgttggtctggggtgtgtggagagatgagcaccgcttcttgggaagcccctccctatgatctgccatcctggagcctcagctgtgtccgctgtggggtgggggctttagggaggaggagagaggacccccagcttgagggagagccagtctaccctttgcccattcgctgccctcggttccctgccagcctctcactgtgtgacccagacaggcccagccccgcagctttcctcccgcagcacctccgtgtccatccagccaaccagtttctcaggcagcagcccccgctcagcaatcactgtcccggggaacacccaatgctccaaggaaggctctgcagctccagggacgggatgatgaggctggccctgatggagcctcgggcctgtgtgctgcaggaggagcccgtggctggcgcacagatggacagggccaagctcttcatggtggctgtgaagcaggaactgagccaagccaactttgccaccttcacccaggccctgcaggactacaagggttccgatgacttcgccgccctggccgcctgtctcggccccctctttgctgaggaccccaagaagcacagcctgctccaaggtgccctggcttgcaggggccgcccatcctgagggcagtgctgccgctgtgtggggtgggggccatctgggtccaaggttgtctctgttctctagagaaaagggggcggatggggacagacaccccttcctctacaggcttctaccagtttgtgcggcctcaccacaaggagcagtttgaggaggtctgtatccagctgacgggacggggctgcggctaccggccagagcacagcgttccccgaaggcagcgggcacagccagccctggaccccactggtaaatggggcctcgggtgggaccctcagactcctccgtggaaggcaccggagtcctgggctggttggggtgggcatccctggggccctgcgtggccccgcctctctgttcccgtgtgggagtgatgggggccaccccccaccaccagcagcagtatcacttgttggggagaccctgtgcaactccacgcagggccctgcccttcccatagcccctgacccctacgcacagccctgtccagctgccgcacgtgtcccctgccccccatgcacagccctggggagtccactgcacacgccagggtcctagacccctagacccctagatccctgtcctcccgtcctccctacttctgcctttgcctagggtggagtccaagcctccagaggtgggagtgtctgtgttcgtgcttgttaacgaacagcccccatagagctcctccagtgtagggttcacccagcggggaacctaacctgttgggaagaccccagaccccttctgggcttggtacccaccctggggctctaagctcctgagcagggctttggcctgcctgccactgttccagcccccatccagcagttggtgtctcctctgctgccccggcacctgggcacgtgtgcctgcctgggttttcccgccctggtccgaggtgggtgaggcctggcctccccagccagcccttccccctccccgggggaactttccaggtgcttgggaccagctttgtggctctccatctcttcatcaggaaggacagcgctagatcccaagctgactgtgtccaaggctgcagcccagcagctggacccccgagagcacctgaaccagggcaggccccacctgtcgcccagaccaccccccacaggtagctgagtcctgaactgtgtgcagcctgtgacttggtgggttcctcagtggcttgatgaggctaacacttgagtgtggcttgagcctttgaaccggaggtgccagtggagctgcccctgtggggagcatctcctggtgggggctcccggttctgtaccccgcagttgtcctgagcagctctccaggagttcgtgggaggaagggcaggcagggtggtgggactctcagtcctctgccccagcgccactctgagccatgctactcccacaccaggaggccctggcagccacccacagcaggggtccagagcgcccagagcagggaagcagggccagcgcgccgtgagcgcctacctggcagatgtccgcagggccctggggtctgcaggctgtagccagctcttggcagcactgacagcctacaagcaagacgatgacctcgacaaggtgctggccgtgctggccgccctgaccactgcaaagccggaggacttccccctgctgcagagcaagtggcctgggcgtgggggacagccagtggggtgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggggcaggggcagggtggggaccgtgccgggtctgagtgaagctccccgcagggttcagcatgtttgtgcgtccacaccacaagcagcgcttctcacagacgtgcacagacctgaccggcagaccctccctgggcacggagccaccaggaccccaggaggagagtcttgccgtgcctcctgtgctcacccacagggctccccaaccaggtagggcgcctgcctggctgctcctgacagtgccccaaccgcacgcagccctgggagtgagcagcaaagccccaggccctgctcagactcagtctccgtctccaggcccctcacggtccgagaagcccggaaagacccagagcgagatctcgaccttccttagacagaggccagcagggactgtgggggcgggcggtgaggctgcagggcccagccagtccccaggacctccccacgggcctgcagcatctgagtggggtgagcctcctgggagagaccttgctgggcagcaggctgcgggagctccgagcgggcccctctcagcaggctgtgtgtgccagggctgtggggcggaggatgtggtgcccttccagtgccctgcctgtgatttccagcgctgccaagcctgttggcaacggcaccttcaggttggtgcctggtcactacagttcctgctgggtgtagccccaggtggtgggctgagggggaaagggccgggtatagccccaggtggtgggctggtggcaacgcctggcaggtgcgtccagcgggccggttgtcttacaggcctctaggacgtgcccatcctgccacaccgcctccaggaagcagagcgtcacgcaggtcttctggccagagccccagtga

SEQ ID NO: 3 - DNA, 18 bp, Homo sapiens
  source 1..18: mol_type = other DNA
gaccactgtccttccatg

SEQ ID NO: 4 - DNA, 46 bp, Homo sapiens
  source 1..46: mol_type = other DNA
ttcagagattcaagttataataaagctcttcttatattgaggggga

SEQ ID NO: 5 - DNA, 21 bp, Homo sapiens
  source 1..21: mol_type = other DNA
gagattcaagttataataaag

SEQ ID NO: 6 - DNA, 18 bp, Homo sapiens
  source 1..18: mol_type = other DNA
aggaatagggttggtttt

SEQ ID NO: 7 - DNA, 17 bp, Homo sapiens
  source 1..17: mol_type = other DNA
ccttactacctgtcccg

SEQ ID NO: 8 - DNA, 20 bp, Homo sapiens
  source 1..20: mol_type = other DNA
acaattacttgttggatgcc

SEQ ID NO: 9 - DNA, 18 bp, Homo sapiens
  source 1..18: mol_type = other DNA
agcttctaacccaaccag

SEQ ID NO: 10 - DNA, 21 bp, Homo sapiens
  source 1..21: mol_type = other DNA
tataaacctaaatgtaaaagc

SEQ ID NO: 11 - DNA, 20 bp, Homo sapiens
  source 1..20: mol_type = other DNA
ttcaccaaaatttaaagctt

SEQ ID NO: 12 - DNA, 26 bp, Homo sapiens
  source 1..26: mol_type = other DNA
ctttgaccagagtatgtaaaattctc

SEQ ID NO: 13 - DNA, 22 bp, Homo sapiens
  source 1..22: mol_type = other DNA
tttgaccagagtatgtaaaatt

SEQ ID NO: 14 - DNA, 18 bp, Homo sapiens
  source 1..18: mol_type = other DNA
tttgaccagagtatgtaa

SEQ ID NO: 15 - DNA, 19 bp, Homo sapiens
  source 1..19: mol_type = other DNA
gaccagagtatgtaaaatt

SEQ ID NO: 16 - DNA, 18 bp, Homo sapiens
  source 1..18: mol_type = other DNA
accagagtatgtaaaatt

SEQ ID NO: 17 - DNA, 18 bp, Homo sapiens
  source 1..18: mol_type = other DNA
aagacgtgttcaaagatt

SEQ ID NO: 18 - DNA, 17 bp, Homo sapiens
  source 1..17: mol_type = other DNA
ggacctactgttttttg

SEQ ID NO: 19 - DNA, 19 bp, Homo sapiens
  source 1..19: mol_type = other DNA
ggacctactgttttattcc

SEQ ID NO: 20 - DNA, 22 bp, Homo sapiens
  source 1..22: mol_type = other DNA
gtcccttctcttcctcctgtag

SEQ ID NO: 21 - DNA, 19 bp, Homo sapiens
  source 1..19: mol_type = other DNA
cgtgatctttgacgaagct

SEQ ID NO: 22 - DNA, 16 bp, Homo sapiens
  source 1..16: mol_type = other DNA
cgcaaacctttctgga

SEQ ID NO: 23 - DNA, 23 bp, Homo sapiens
  source 1..23: mol_type = other DNA
agcctgtgtgtggagtatgagca

SEQ ID NO: 24 - DNA, 20 bp, Homo sapiens
  source 1..20: mol_type = other DNA
cgtttccgtgttggtctggg

SEQ ID NO: 25 - DNA, 19 bp, Homo sapiens
  source 1..19: mol_type = other DNA
gactacaagggttccgatg

SEQ ID NO: 26 - DNA, 20 bp, Homo sapiens
  source 1..20: mol_type = other DNA
agtttgaggaggtctgtatc

SEQ ID NO: 27 - DNA, 17 bp, synthetic construct
  misc_feature 1..17: note = Synthetic oligonucleotide
  source 1..17: mol_type = other DNA
catggaaggacagtggt

SEQ ID NO: 28 - DNA, 20 bp, synthetic construct
  misc_feature 1..20: note = Synthetic oligonucleotide
  source 1..20: mol_type = other DNA
agctttattataacttgaat

SEQ ID NO: 29 - DNA, 16 bp, synthetic construct
  misc_feature 1..16: note = Synthetic oligonucleotide
  source 1..16: mol_type = other DNA
cgggacaggtagtaag

SEQ ID NO: 30 - DNA, 15 bp, synthetic construct
  misc_feature 1..15: note = Synthetic oligonucleotide
  source 1..15: mol_type = other DNA
cgggacaggtagtaa

SEQ ID NO: 31 - DNA, 19 bp, synthetic construct
  misc_feature 1..19: note = Synthetic oligonucleotide
  source 1..19: mol_type = other DNA
gcatccaacaagtaattgt

SEQ ID NO: 32 - DNA, 18 bp, synthetic construct
  misc_feature 1..18: note = Synthetic oligonucleotide
  source 1..18: mol_type = other DNA
gcatccaacaagtaattg

SEQ ID NO: 33 - DNA, 18 bp, synthetic construct
  misc_feature 1..18: note = Synthetic oligonucleotide
  source 1..18: mol_type = other DNA
ggcatccaacaagtaatt

SEQ ID NO: 34 - DNA, 16 bp, synthetic construct
  misc_feature 1..16: note = Synthetic oligonucleotide
  source 1..16: mol_type = other DNA
ggttgggttagaagct

SEQ ID NO: 35 - DNA, 20 bp, synthetic construct
  misc_feature 1..20: note = Synthetic oligonucleotide
  source 1..20: mol_type = other DNA
gcttttacatttaggtttat

SEQ ID NO: 36 - DNA, 19 bp, synthetic construct
  misc_feature 1..19: note = Synthetic oligonucleotide
  source 1..19: mol_type = other DNA
catgttcctttctataact

SEQ ID NO: 37 - DNA, 19 bp, synthetic construct
  misc_feature 1..19: note = Synthetic oligonucleotide
  source 1..19: mol_type = other DNA
agctttaaattttggtgaa

SEQ ID NO: 38 - DNA, 20 bp, synthetic construct
  misc_feature 1..20: note = Synthetic oligonucleotide
  source 1..20: mol_type = other DNA
ttttacatactctggtcaaa

SEQ ID NO: 39 - DNA, 18 bp, synthetic construct
  misc_feature 1..18: note = Synthetic oligonucleotide
  source 1..18: mol_type = other DNA
ttttacatactctggtca

SEQ ID NO: 40 - DNA, 20 bp, synthetic construct
  misc_feature 1..20: note = Synthetic oligonucleotide
  source 1..20: mol_type = other DNA
gaattttacatactctggtc

SEQ ID NO: 41 - DNA, 19 bp, synthetic construct
  misc_feature 1..19: note = Synthetic oligonucleotide
  source 1..19: mol_type = other DNA
gaattttacatactctggt

SEQ ID NO: 42 - DNA, 20 bp, synthetic construct
  misc_feature 1..20: note = Synthetic oligonucleotide
  source 1..20: mol_type = other DNA
gagaattttacatactctgg

SEQ ID NO: 43 - DNA, 17 bp, synthetic construct
  misc_feature 1..17: note = Synthetic oligonucleotide
  source 1..17: mol_type = other DNA
atctttgaacacgtctt

SEQ ID NO: 44 - DNA, 18 bp, synthetic construct
  misc_feature 1..18: note = Synthetic oligonucleotide
  source 1..18: mol_type = other DNA
acaaaaaacagtaggtcc

SEQ ID NO: 45 - DNA, 18 bp, synthetic construct
  misc_feature 1..18: note = Synthetic oligonucleotide
  source 1..18: mol_type = other DNA
ggaataaaacagtaggtc

SEQ ID NO: 46 - DNA, 18 bp, synthetic construct
  misc_feature 1..18: note = Synthetic oligonucleotide
  source 1..18: mol_type = other DNA
agcttcgtcaaagatcac

SEQ ID NO: 47 - DNA, 16 bp, synthetic construct
  misc_feature 1..16: note = Synthetic oligonucleotide
  source 1..16: mol_type = other DNA
ggtgggtggatgtttc

SEQ ID NO: 48 - DNA, 16 bp, synthetic construct
  misc_feature 1..16: note = Synthetic oligonucleotide
  source 1..16: mol_type = other DNA
ggtggtgtggagaagc

SEQ ID NO: 49 - DNA, 17 bp, synthetic construct
  misc_feature 1..17: note = Synthetic oligonucleotide
  source 1..17: mol_type = other DNA
gctcatactccacacac

SEQ ID NO: 50 - DNA, 20 bp, synthetic construct
  misc_feature 1..20: note = Synthetic oligonucleotide
  source 1..20: mol_type = other DNA
catcggaacccttgtagtcc

SEQ ID NO: 51 - DNA, 19 bp, synthetic construct
  misc_feature 1..19: note = Synthetic oligonucleotide
  source 1..19: mol_type = other DNA
gatacagacctcctcaaac

SEQ ID NO: 52 - DNA, 17 bp, synthetic construct
  misc_feature 1..17: note = Synthetic oligonucleotide
  source 1..17: mol_type = other DNA
ggtggaggtggtgctgc

SEQ ID NO: 53 - DNA, 16 bp, synthetic construct
  misc_feature 1..16: note = Synthetic oligonucleotide
  source 1..16: mol_type = other DNA
aggtggaggtggtgct

SEQ ID NO: 54 - DNA, 16 bp, synthetic construct
  misc_feature 1..16: note = Synthetic oligonucleotide
  source 1..16: mol_type = other DNA
tggtgtgggagtagca

SEQ ID NO: 55 - DNA, 16 bp, synthetic construct
  misc_feature 1..16: note = Synthetic oligonucleotide
  source 1..16: mol_type = other DNA
cgatggcgagaaatta

SEQ ID NO: 56 - DNA, 20 bp, synthetic construct
  misc_feature 1..20: note = Synthetic oligonucleotide
  source 1..20: mol_type = other DNA
taattcagcaaaaaagccca

SEQ ID NO: 57 - DNA, 17 bp, synthetic construct
  misc_feature 1..17: note = Synthetic oligonucleotide
  source 1..17: mol_type = other DNA
aagaatctgacacccca

SEQ ID NO: 58 - DNA, 21 bp, synthetic construct
  misc_feature 1..21: note = Synthetic oligonucleotide
  source 1..21: mol_type = other DNA
agacagccaagaatctgacac

SEQ ID NO: 59 - DNA, 17 bp, synthetic construct
  misc_feature 1..17: note = Synthetic oligonucleotide
  source 1..17: mol_type = other DNA
cagccaagaatctgaca

SEQ ID NO: 60 - DNA, 15 bp, synthetic construct
  misc_feature 1..15: note = Synthetic oligonucleotide
  source 1..15: mol_type = other DNA
acaggaacccgacag

SEQ ID NO: 61 - DNA, 21 bp, synthetic construct
  misc_feature 1..21: note = Synthetic oligonucleotide
  source 1..21: mol_type = other DNA
gttactctcttgtttcttcac

SEQ ID NO: 62 - DNA, 19 bp, synthetic construct
  misc_feature 1..19: note = Synthetic oligonucleotide
  source 1..19: mol_type = other DNA
ttactctcttgtttcttca

SEQ ID NO: 63 - DNA, 19 bp, synthetic construct
  misc_feature 1..19: note = Synthetic oligonucleotide
  source 1..19: mol_type = other DNA
gttactctcttgtttcttc

SEQ ID NO: 64 - DNA, 16 bp, synthetic construct
  misc_feature 1..16: note = Synthetic oligonucleotide
  source 1..16: mol_type = other DNA
cgtgggtggagaagca

SEQ ID NO: 65 - DNA, 16 bp, synthetic construct
  misc_feature 1..16: note = Synthetic oligonucleotide
  source 1..16: mol_type = other DNA
acgtgggtggagaagc

SEQ ID NO: 66 - DNA, 17 bp, synthetic construct
  misc_feature 1..17: note = Synthetic oligonucleotide
  source 1..17: mol_type = other DNA
cagaaactgtaagggca

SEQ ID NO: 67 - DNA, 17 bp, synthetic construct
  misc_feature 1..17: note = Synthetic oligonucleotide
  source 1..17: mol_type = other DNA
agggatagcagggaagg

SEQ ID NO: 68 - DNA, 17 bp, synthetic construct
  misc_feature 1..17: note = Synthetic oligonucleotide
  source 1..17: mol_type = other DNA
gcttaaacacagacaga

SEQ ID NO: 69 - DNA, 17 bp, synthetic construct
  misc_feature 1..17: note = Synthetic oligonucleotide
  source 1..17: mol_type = other DNA
tgcttaaacacagacag

SEQ ID NO: 70 - DNA, 18 bp, synthetic construct
  misc_feature 1..18: note = Synthetic oligonucleotide
  source 1..18: mol_type = other DNA
cagggcagggaagaacag

SEQ ID NO: 71 - DNA, 16 bp, synthetic construct
  misc_feature 1..16: note = Synthetic oligonucleotide
  source 1..16: mol_type = other DNA
catggaaggacagtgg

SEQ ID NO: 72 - DNA, 17 bp, synthetic construct
  misc_feature 1..17: note = Synthetic oligonucleotide
  source 1..17: mol_type = other DNA
ccccctcaatataagaa

SEQ ID NO: 73 - DNA, 18 bp, synthetic construct
  misc_feature 1..18: note = Synthetic oligonucleotide
  source 1..18: mol_type = other DNA
aaccaaccctattcctgg

SEQ ID NO: 74 - DNA, 16 bp, synthetic construct
  misc_feature 1..16: note = Synthetic oligonucleotide
  source 1..16: mol_type = other DNA
accaaccctattcctg

SEQ ID NO: 75 - DNA, 17 bp, synthetic construct
  misc_feature 1..17: note = Synthetic oligonucleotide
  source 1..17: mol_type = other DNA
aaccaaccctattcctg

SEQ ID NO: 76 - DNA, 18 bp, synthetic construct
  misc_feature 1..18: note = Synthetic oligonucleotide
  source 1..18: mol_type = other DNA
aaaaccaaccctattcct

SEQ ID NO: 77 - DNA, 16 bp, synthetic construct
  misc_feature 1..16: note = Synthetic oligonucleotide
  source 1..16: mol_type = other DNA
aaaccaaccctattcc

SEQ ID NO: 78 - DNA, 17 bp, synthetic construct
  misc_feature 1..17: note = Synthetic oligonucleotide
  source 1..17: mol_type = other DNA
ggtagtaagggcacacc

SEQ ID NO: 79 - DNA, 20 bp, synthetic construct
  misc_feature 1..20: note = Synthetic oligonucleotide
  source 1..20: mol_type = other DNA
gacaggtagtaagggcacac

SEQ ID NO: 80 - DNA, 15 bp, synthetic construct
  misc_feature 1..15: note = Synthetic oligonucleotide
  source 1..15: mol_type = other DNA
gtagtaagggcacac

SEQ ID NO: 81 - DNA, 19 bp, synthetic construct
  misc_feature 1..19: note = Synthetic oligonucleotide
  source 1..19: mol_type = other DNA
gacaggtagtaagggcaca

SEQ ID NO: 82 - DNA, 18 bp, synthetic construct
  misc_feature 1..18: note = Synthetic oligonucleotide
  source 1..18: mol_type = other DNA
acaggtagtaagggcaca

SEQ ID NO: 83 - DNA, 17 bp, synthetic construct
  misc_feature 1..17: note = Synthetic oligonucleotide
  source 1..17: mol_type = other DNA
gacaggtagtaagggca

SEQ ID NO: 84 - DNA, 18 bp, synthetic construct
  misc_feature 1..18: note = Synthetic oligonucleotide
  source 1..18: mol_type = other DNA
cgggacaggtagtaaggg

SEQ ID NO: 85 - DNA, 18 bp, synthetic construct
  misc_feature 1..18: note = Synthetic oligonucleotide
  source 1..18: mol_type = other DNA
catccaacaagtaattgt

SEQ ID NO: 86 - DNA, 20 bp, synthetic construct
  misc_feature 1..20: note = Synthetic oligonucleotide
  source 1..20: mol_type = other DNA
ggcatccaacaagtaattgt

SEQ ID NO: 87 - DNA, 19 bp, synthetic construct
  misc_feature 1..19: note = Synthetic oligonucleotide
  source 1..19: mol_type = other DNA
ggcatccaacaagtaattg

SEQ ID NO: 88 - DNA, 17 bp, synthetic construct
  misc_feature 1..17: note = Synthetic oligonucleotide
  source 1..17: mol_type = other DNA
ggcatccaacaagtaat

SEQ ID NO: 89 - DNA, 17 bp, synthetic construct
  misc_feature 1..17: note = Synthetic oligonucleotide
  source 1..17: mol_type = other DNA
cgtgaaggagagaacct

SEQ ID NO: 90 - DNA, 17 bp, synthetic construct
  misc_feature 1..17: note = Synthetic oligonucleotide
  source 1..17: mol_type = other DNA
acgtgaaggagagaacc

SEQ ID NO: 91 - DNA, 18 bp, synthetic construct
  misc_feature 1..18: note = Synthetic oligonucleotide
  source 1..18: mol_type = other DNA
gacgtgaaggagagaacc

SEQ ID NO: 92 - DNA, 17 bp, synthetic construct
  misc_feature 1..17: note = Synthetic oligonucleotide
  source 1..17: mol_type = other DNA
gacgtgaaggagagaac

SEQ ID NO: 93 - DNA, 16 bp, synthetic construct
  misc_feature 1..16: note = Synthetic oligonucleotide
  source 1..16: mol_type = other DNA
cagtcttgctatgcct

SEQ ID NO: 94 - DNA, 17 bp, synthetic construct
  misc_feature 1..17: note = Synthetic oligonucleotide
  source 1..17: mol_type = other DNA
ctagaatcaaagctcca

SEQ ID NO: 95 - DNA, 15 bp, synthetic construct
  misc_feature 1..15: note = Synthetic oligonucleotide
  source 1..15: mol_type = other DNA
acatcgcacttgggc

SEQ ID NO: 96 - DNA, 16 bp, synthetic construct
  misc_feature 1..16: note = Synthetic oligonucleotide
  source 1..16: mol_type = other DNA
cacggcaaacctcacc

SEQ ID NO: 97 - DNA, 18 bp, synthetic construct
  misc_feature 1..18: note = Synthetic oligonucleotide
  source 1..18: mol_type = other DNA
aaccacggcaaacctcac

SEQ ID NO: 98 - DNA, 17 bp, synthetic construct
  misc_feature 1..17: note = Synthetic oligonucleotide
  source 1..17: mol_type = other DNA
caaagcaccgagtcacc

SEQ ID NO: 99 - DNA, 17 bp, synthetic construct
  misc_feature 1..17: note = Synthetic oligonucleotide
  source 1..17: mol_type = other DNA
tcaaagcaccgagtcac

SEQ ID NO: 100 - DNA, 16 bp, synthetic construct
  misc_feature 1..16: note = Synthetic oligonucleotide
  source 1..16: mol_type = other DNA
ctggttgggttagaag

SEQ ID NO: 101 - DNA, 18 bp, synthetic construct
  misc_feature 1..18: note = Synthetic oligonucleotide
  source 1..18: mol_type = other DNA
tataacttttagtttagc

SEQ ID NO: 102 - DNA, 18 bp, synthetic construct
  misc_feature 1..18: note = Synthetic oligonucleotide
  source 1..18: mol_type = other DNA
ttcctttctataactttt

SEQ ID NO: 103 - DNA, 19 bp, synthetic construct
  misc_feature 1..19: note = Synthetic oligonucleotide
  source 1..19: mol_type = other DNA
gttcctttctataactttt

SEQ ID NO: 104 - DNA, 18 bp, synthetic construct
  misc_feature 1..18: note = Synthetic oligonucleotide
  source 1..18: mol_type = other DNA
gttcctttctataacttt

SEQ ID NO: 105 - DNA, 20 bp, synthetic construct
  misc_feature 1..20: note = Synthetic oligonucleotide
  source 1..20: mol_type = other DNA
atgttcctttctataacttt

SEQ ID NO: 106 - DNA, 19 bp, synthetic construct
  misc_feature 1..19: note = Synthetic oligonucleotide
  source 1..19: mol_type = other DNA
atgttcctttctataactt

SEQ ID NO: 107 - DNA, 18 bp, synthetic construct
  misc_feature 1..18: note = Synthetic oligonucleotide
  source 1..18: mol_type = other DNA
atgttcctttctataact

SEQ ID NO: 108 - DNA, 16 bp, synthetic construct
  misc_feature 1..16: note = Synthetic oligonucleotide
  source 1..16: mol_type = other DNA
gctttaatctgccttc

SEQ ID NO: 109 - DNA, 17 bp, synthetic construct
  misc_feature 1..17: note = Synthetic oligonucleotide
  source 1..17: mol_type = other DNA
ccgtggctttaatctgc

SEQ ID NO: 110 - DNA, 16 bp, synthetic construct
  misc_feature 1..16: note = Synthetic oligonucleotide
  source 1..16: mol_type = other DNA
ccgtggctttaatctg

SEQ ID NO: 111 - DNA, 17 bp, synthetic construct
  misc_feature 1..17: note = Synthetic oligonucleotide
  source 1..17: mol_type = other DNA
caaaaaacagtaggtcc

SEQ ID NO: 112 - DNA, 18 bp, synthetic construct
  misc_feature 1..18: note = Synthetic oligonucleotide
  source 1..18: mol_type = other DNA
gaataaaacagtaggtcc

SEQ ID NO: 113 - DNA, 19 bp, synthetic construct
  misc_feature 1..19: note = Synthetic oligonucleotide
  source 1..19: mol_type = other DNA
ggaataaaacagtaggtcc

SEQ ID NO: 114 - DNA, 17 bp, synthetic construct
  misc_feature 1..17: note = Synthetic oligonucleotide
  source 1..17: mol_type = other DNA
ggaataaaacagtaggt

SEQ ID NO: 115 - DNA, 16 bp, synthetic construct
  misc_feature 1..16: note = Synthetic oligonucleotide
  source 1..16: mol_type = other DNA
ggaataaaacagtagg

SEQ ID NO: 116 - DNA, 16 bp, synthetic construct
  misc_feature 1..16: note = Synthetic oligonucleotide
  source 1..16: mol_type = other DNA
cacagagtgtcatggg

SEQ ID NO: 117 - DNA, 18 bp, synthetic construct
  misc_feature 1..18: note = Synthetic oligonucleotide
  source 1..18: mol_type = other DNA
acagcatggaaaggcacg

SEQ ID NO: 118 - DNA, 17 bp, synthetic construct
  misc_feature 1..17: note = Synthetic oligonucleotide
  source 1..17: mol_type = other DNA
cagcatggaaaggcacg

SEQ ID NO: 119 - DNA, 21 bp, synthetic construct
  misc_feature 1..21: note = Synthetic oligonucleotide
  source 1..21: mol_type = other DNA
tacaggaggaagagaagggac

SEQ ID NO: 120 - DNA, 18 bp, synthetic construct
  misc_feature 1..18: note = Synthetic oligonucleotide
  source 1..18: mol_type = other DNA
acaggaggaagagaaggg

SEQ ID NO: 121 - DNA, 18 bp, synthetic construct
  misc_feature 1..18: note = Synthetic oligonucleotide
  source 1..18: mol_type = other DNA
tctacaggaggaagagaa

SEQ ID NO: 122 - DNA, 17 bp, synthetic construct
  misc_feature 1..17: note = Synthetic oligonucleotide
  source 1..17: mol_type = other DNA
tctacaggaggaagaga

SEQ ID NO: 123 - DNA, 17 bp, synthetic construct
  misc_feature 1..17: note = Synthetic oligonucleotide
  source 1..17: mol_type = other DNA
cttcgtcaaagatcacg

SEQ ID NO: 124 - DNA, 18 bp, synthetic construct
  misc_feature 1..18: note = Synthetic oligonucleotide
  source 1..18: mol_type = other DNA
gcttcgtcaaagatcacg

SEQ ID NO: 125 - DNA, 17 bp, synthetic construct
  misc_feature 1..17: note = Synthetic oligonucleotide
  source 1..17: mol_type = other DNA
gcttcgtcaaagatcac

SEQ ID NO: 126 - DNA, 15 bp, synthetic construct
  misc_feature 1..15: note = Synthetic oligonucleotide
  source 1..15: mol_type = other DNA
ccagaaaggtttgcg

SEQ ID NO: 127 - DNA, 16 bp, synthetic construct
  misc_feature 1..16: note = Synthetic oligonucleotide
  source 1..16: mol_type = other DNA
tccagaaaggtttgcg

SEQ ID NO: 128 - DNA, 17 bp, synthetic construct
  misc_feature 1..17: note = Synthetic oligonucleotide
  source 1..17: mol_type = other DNA
cagaggcatcggatcag

SEQ ID NO: 129 - DNA, 16 bp, synthetic construct
  misc_feature 1..16: note = Synthetic oligonucleotide
  source 1..16: mol_type = other DNA
cagaggcatcggatca

SEQ ID NO: 130 - DNA, 17 bp, synthetic construct
  misc_feature 1..17: note = Synthetic oligonucleotide
  source 1..17: mol_type = other DNA
agcagaggcatcggatc

SEQ ID NO: 131 - DNA, 17 bp, synthetic construct
  misc_feature 1..17: note = Synthetic oligonucleotide
  source 1..17: mol_type = other DNA
attcttcacacatcttc

SEQ ID NO: 132 - DNA, 15 bp, synthetic construct
  misc_feature 1..15: note = Synthetic oligonucleotide
  source 1..15: mol_type = other DNA
ctatgaacgcacctg

SEQ ID NO: 133 - DNA, 17 bp, synthetic construct
  misc_feature 1..17: note = Synthetic oligonucleotide
  source 1..17: mol_type = other DNA
ggctatgaacgcacctg

SEQ ID NO: 134 - DNA, 17 bp, synthetic construct
  misc_feature 1..17: note = Synthetic oligonucleotide
  source 1..17: mol_type = other DNA
gctgggagaagacatag

SEQ ID NO: 135 - DNA, 19 bp, synthetic construct
  misc_feature 1..19: note = Synthetic oligonucleotide
  source 1..19: mol_type = other DNA
caaaatgcccttacagtga

SEQ ID NO: 136 - DNA, 17 bp, synthetic construct
  misc_feature 1..17: note = Synthetic oligonucleotide
  source 1..17: mol_type = other DNA
caaaatgcccttacagt

SEQ ID NO: 137 - DNA, 21 bp, synthetic construct
  misc_feature 1..21: note = Synthetic oligonucleotide
  source 1..21: mol_type = other DNA
tgtgcgattttaaaggaaaat

SEQ ID NO: 138 - DNA, 21 bp, synthetic construct
  misc_feature 1..21: note = Synthetic oligonucleotide
  source 1..21: mol_type = other DNA
catgtgcgattttaaaggaaa

SEQ ID NO: 139 - DNA, 18 bp, synthetic construct
  misc_feature 1..18: note = Synthetic oligonucleotide
  source 1..18: mol_type = other DNA
tgtgcgattttaaaggaa

SEQ ID NO: 140 - DNA, 19 bp, synthetic construct
  misc_feature 1..19: note = Synthetic oligonucleotide
  source 1..19: mol_type = other DNA
catgtgcgattttaaagga

SEQ ID NO: 141 - DNA, 18 bp, synthetic construct
  misc_feature 1..18: note = Synthetic oligonucleotide
  source 1..18: mol_type = other DNA
atgtgcgattttaaagga

SEQ ID NO: 142 - DNA, 21 bp, synthetic construct
  misc_feature 1..21: note = Synthetic oligonucleotide
  source 1..21: mol_type = other DNA
accctgtcacttaaatatatg

SEQ ID NO: 143 - DNA, 17 bp, synthetic construct
  misc_feature 1..17: note = Synthetic oligonucleotide
  source 1..17: mol_type = other DNA
gagggaggtggagcgtt

SEQ ID NO: 144 - DNA, 17 bp, synthetic construct
  misc_feature 1..17: note = Synthetic oligonucleotide
  source 1..17: mol_type = other DNA
ctgaagagtggagaagg

SEQ ID NO: 145 - DNA, 20 bp, synthetic construct
  misc_feature 1..20: note = Synthetic oligonucleotide
  source 1..20: mol_type = other DNA
caataaataaagtgtgagga

SEQ ID NO: 146 - DNA, 18 bp, synthetic construct
  misc_feature 1..18: note = Synthetic oligonucleotide
  source 1..18: mol_type = other DNA
caacccagtaaccatgac

SEQ ID NO: 147 - DNA, 17 bp, synthetic construct
  misc_feature 1..17: note = Synthetic oligonucleotide
  source 1..17: mol_type = other DNA
caacccagtaaccatga

SEQ ID NO: 148 - DNA, 19 bp, synthetic construct
  misc_feature 1..19: note = Synthetic oligonucleotide
  source 1..19: mol_type = other DNA
accaacccagtaaccatga

SEQ ID NO: 149 - DNA, 16 bp, synthetic construct
  misc_feature 1..16: note = Synthetic oligonucleotide
  source 1..16: mol_type = other DNA
gagcaggtgttttatc

SEQ ID NO: 150 - DNA, 17 bp, synthetic construct
  misc_feature 1..17: note = Synthetic oligonucleotide
  source 1..17: mol_type = other DNA
ggtcgaggaggtgtcac

SEQ ID NO: 151 - DNA, 16 bp, synthetic construct
  misc_feature 1..16: note = Synthetic oligonucleotide
  source 1..16: mol_type = other DNA
gtcgaggaggtgtcac

SEQ ID NO: 152 - DNA, 16 bp, synthetic construct
  misc_feature 1..16: note = Synthetic oligonucleotide
  source 1..16: mol_type = other DNA
ggtcgaggaggtgtca

SEQ ID NO: 153 - DNA, 15 bp, synthetic construct
  misc_feature 1..15: note = Synthetic oligonucleotide
  source 1..15: mol_type = other DNA
ggtcgaggaggtgtc

SEQ ID NO: 154 - DNA, 17 bp, synthetic construct
  misc_feature 1..17: note = Synthetic oligonucleotide
  source 1..17: mol_type = other DNA
ccaggtctcaaaaaggg

SEQ ID NO: 155 - DNA, 15 bp, synthetic construct
  misc_feature 1..15: note = Synthetic oligonucleotide
  source 1..15: mol_type = other DNA
attacgctgaggaca

SEQ ID NO: 156 - DNA, 15 bp, synthetic construct
  misc_feature 1..15: note = Synthetic oligonucleotide
  source 1..15: mol_type = other DNA
cattacgctgaggac

SEQ ID NO: 157 - DNA, 14 bp, synthetic construct
  misc_feature 1..14: note = Synthetic oligonucleotide
  source 1..14: mol_type = other DNA
cttgagcattacgc

SEQ ID NO: 158 - DNA, 17 bp, synthetic construct
  misc_feature 1..17: note = Synthetic oligonucleotide
  source 1..17: mol_type = other DNA
cgaggagaagaaggcag

SEQ ID NO: 159 - DNA, 19 bp, synthetic construct
  misc_feature 1..19: note = Synthetic oligonucleotide
  source 1..19: mol_type = other DNA
ccttggtctgaaacgtgat

SEQ ID NO: 160 - DNA, 15 bp, synthetic construct
  misc_feature 1..15: note = Synthetic oligonucleotide
  source 1..15: mol_type = other DNA
ctaacgcctccacgc

SEQ ID NO: 161 - DNA, 15 bp, synthetic construct
  misc_feature 1..15: note = Synthetic oligonucleotide
  source 1..15: mol_type = other DNA
ggacaggctctacgg

SEQ ID NO: 162 - DNA, 20 bp, synthetic construct
  misc_feature 1..20: note = Synthetic oligonucleotide
  source 1..20: mol_type = other DNA
actaatacagcaggagaagg

SEQ ID NO: 163 - DNA, 21 bp, synthetic construct
  misc_feature 1..21: note = Synthetic oligonucleotide
  source 1..21: mol_type = other DNA
aactaatacagcaggagaagg

SEQ ID NO: 164 - DNA, 21 bp, synthetic construct
  misc_feature 1..21: note = Synthetic oligonucleotide
  source 1..21: mol_type = other DNA
taactaatacagcaggagaag

SEQ ID NO: 165 - DNA, 16 bp, synthetic construct
  misc_feature 1..16: note = Synthetic oligonucleotide
  source 1..16: mol_type = other DNA
ttgaagagccaaccac

SEQ ID NO: 166 - DNA, 17 bp, synthetic construct
  misc_feature 1..17: note = Synthetic oligonucleotide
  source 1..17: mol_type = other DNA
ccattttcactgtcaag

SEQ ID NO: 167 - DNA, 17 bp, synthetic construct
  misc_feature 1..17: note = Synthetic oligonucleotide
  source 1..17: mol_type = other DNA
gccattttcactgtcaa

SEQ ID NO: 168 - DNA, 16 bp, synthetic construct
  misc_feature 1..16: note = Synthetic oligonucleotide
  source 1..16: mol_type = other DNA
agaaatgcggagaagc

SEQ ID NO: 169 - DNA, 20 bp, synthetic construct
  misc_feature 1..20: note = Synthetic oligonucleotide
  source 1..20: mol_type = other DNA
aaatggaaaaaatgaccagc

SEQ ID NO: 170 - DNA, 20 bp, synthetic construct
  misc_feature 1..20: note = Synthetic oligonucleotide
  source 1..20: mol_type = other DNA
aggacttacgacaaaaccac

SEQ ID NO: 171 - DNA, 18 bp, synthetic construct
  misc_feature 1..18: note = Synthetic oligonucleotide
  source 1..18: mol_type = other DNA
ggacttacgacaaaacca

SEQ ID NO: 172 - DNA, 17 bp, synthetic construct
  misc_feature 1..17: note = Synthetic oligonucleotide
  source 1..17: mol_type = other DNA
gacttacgacaaaacca

SEQ ID NO: 173 - DNA, 18 bp, synthetic construct
  misc_feature 1..18: note = Synthetic oligonucleotide
  source 1..18: mol_type = other DNA
acaccaggacttacgaca

SEQ ID NO: 174 - DNA, 17 bp, synthetic construct
  misc_feature 1..17: note = Synthetic oligonucleotide
  source 1..17: mol_type = other DNA
tagaaattcaacatggc

SEQ ID NO: 175 - DNA, 18 bp, synthetic construct
  misc_feature 1..18: note = Synthetic oligonucleotide
  source 1..18: mol_type = other DNA
ctagaaattcaacatggc

SEQ ID NO: 176 - DNA, 14 bp, synthetic construct
  misc_feature 1..14: note = Synthetic oligonucleotide
  source 1..14: mol_type = other DNA
gtcatcggttcacc

SEQ ID NO: 177 - DNA, 14 bp, synthetic construct
  misc_feature 1..14: note = Synthetic oligonucleotide
  source 1..14: mol_type = other DNA
actcgaagacgcca

SEQ ID NO: 178 - DNA, 14 bp, synthetic construct
  misc_feature 1..14: note = Synthetic oligonucleotide
  source 1..14: mol_type = other DNA
gactcgaagacgcc

SEQ ID NO: 179 - DNA, 17 bp, synthetic construct
  misc_feature 1..17: note = Synthetic oligonucleotide
  source 1..17: mol_type = other DNA
ggcacaagcagaacgac

SEQ ID NO: 180 - DNA, 18 bp, synthetic construct
  misc_feature 1..18: note = Synthetic oligonucleotide
  source 1..18: mol_type = other DNA
agtcagaacaaaggaggc

SEQ ID NO: 181 - DNA, 18 bp, synthetic construct
  misc_feature 1..18: note = Synthetic oligonucleotide
  source 1..18: mol_type = other DNA
gaagtcagaacaaaggag

SEQ ID NO: 182 - DNA, 19 bp, synthetic construct
  misc_feature 1..19: note = Synthetic oligonucleotide
  source 1..19: mol_type = other DNA
gcagaagtcagaacaaagg

SEQ ID NO: 183 - DNA, 19 bp, synthetic construct
  misc_feature 1..19: note = Synthetic oligonucleotide
  source 1..19: mol_type = other DNA
gtgcagaagtcagaacaaa

SEQ ID NO: 184 - DNA, 18 bp, synthetic construct
  misc_feature 1..18: note = Synthetic oligonucleotide
  source 1..18: mol_type = other DNA
gtgcagaagtcagaacaa

SEQ ID NO: 185 - DNA, 18 bp, synthetic construct
  misc_feature 1..18: note = Synthetic oligonucleotide
  source 1..18: mol_type = other DNA
aaggatgagggagcggac

SEQ ID NO: 186 - DNA, 16 bp, synthetic construct
  misc_feature 1..16: note = Synthetic oligonucleotide
  source 1..16: mol_type = other DNA
gtaaggatgagggagc

SEQ ID NO: 187 - DNA, 17 bp, synthetic construct
  misc_feature 1..17: note = Synthetic oligonucleotide
  source 1..17: mol_type = other DNA
tggtaaggatgagggag

SEQ ID NO: 188 - DNA, 16 bp, synthetic construct
  misc_feature 1..16: note = Synthetic oligonucleotide
  source 1..16: mol_type = other DNA
cgtacatctgcatctc

SEQ ID NO: 189 - DNA, 21 bp, synthetic construct
  misc_feature 1..21: note = Synthetic oligonucleotide
  source 1..21: mol_type = other DNA
tgtaagataagaggcaacact

SEQ ID NO: 190 - DNA, 21 bp, synthetic construct
  misc_feature 1..21: note = Synthetic oligonucleotide
  source 1..21: mol_type = other DNA
ttgtaagataagaggcaacac

SEQ ID NO: 191 - DNA, 20 bp, synthetic construct
  misc_feature 1..20: note = Synthetic oligonucleotide
  source 1..20: mol_type = other DNA
ttgtaagataagaggcaaca

SEQ ID NO: 192 - DNA, 21 bp, synthetic construct
  misc_feature 1..21: note = Synthetic oligonucleotide
  source 1..21: mol_type = other DNA
tttgtaagataagaggcaaca

SEQ ID NO: 193 - DNA, 17 bp, synthetic construct
  misc_feature 1..17: note = Synthetic oligonucleotide
  source 1..17: mol_type = other DNA
tgtaagataagaggcaa

SEQ ID NO: 194 - DNA, 17 bp, synthetic construct
  misc_feature 1..17: note = Synthetic oligonucleotide
  source 1..17: mol_type = other DNA
ctggaaggaaagttggt

SEQ ID NO: 195 - DNA, 19 bp, synthetic construct
  misc_feature 1..19: note = Synthetic oligonucleotide
  source 1..19: mol_type = other DNA
atagtaagcactgatggtc

SEQ ID NO: 196 - DNA, 16 bp, synthetic construct
  misc_feature 1..16: note = Synthetic oligonucleotide
  source 1..16: mol_type = other DNA
tagtaagcactgatgg

SEQ ID NO: 197 - DNA, 17 bp, synthetic construct
  misc_feature 1..17: note = Synthetic oligonucleotide
  source 1..17: mol_type = other DNA
catagtaagcactgatg

SEQ ID NO: 198 - DNA, 16 bp, synthetic construct
  misc_feature 1..16: note = Synthetic oligonucleotide
  source 1..16: mol_type = other DNA
ctgtaactcacctggc

SEQ ID NO: 199 - DNA, 15 bp, synthetic construct
  misc_feature 1..15: note = Synthetic oligonucleotide
  source 1..15: mol_type = other DNA
cggatcactcgcccg

SEQ ID NO: 200 - DNA, 17 bp, synthetic construct
  misc_feature 1..17: note = Synthetic oligonucleotide
  source 1..17: mol_type = other DNA
acacaggctactctcgg

SEQ ID NO: 201 - DNA, 16 bp, synthetic construct
  misc_feature 1..16: note = Synthetic oligonucleotide
  source 1..16: mol_type = other DNA
acacaggctactctcg

SEQ ID NO: 202 - DNA, 17 bp, synthetic construct
  misc_feature 1..17: note = Synthetic oligonucleotide
  source 1..17: mol_type = other DNA
ccacacacaggctactc

SEQ ID NO: 203 - DNA, 18 bp, synthetic construct
  misc_feature 1..18: note = Synthetic oligonucleotide
  source 1..18: mol_type = other DNA
atactccacacacaggct

SEQ ID NO: 204 - DNA, 17 bp, synthetic construct
  misc_feature 1..17: note = Synthetic oligonucleotide
  source 1..17: mol_type = other DNA
atactccacacacaggc

SEQ ID NO: 205 - DNA, 19 bp, synthetic construct
  misc_feature 1..19: note = Synthetic oligonucleotide
  source 1..19: mol_type = other DNA
gctcatactccacacacag

SEQ ID NO: 206 - DNA, 17 bp, synthetic construct
  misc_feature 1..17: note = Synthetic oligonucleotide
  source 1..17: mol_type = other DNA
tcatactccacacacag

SEQ ID NO: 207 - DNA, 18 bp, synthetic construct
  misc_feature 1..18: note = Synthetic oligonucleotide
  source 1..18: mol_type = other DNA
gctcatactccacacaca

SEQ ID NO: 208 - DNA, 18 bp, synthetic construct
  misc_feature 1..18: note = Synthetic oligonucleotide
  source 1..18: mol_type = other DNA
tgctcatactccacacac

SEQ ID NO: 209 - DNA, 19 bp, synthetic construct
  misc_feature 1..19: note = Synthetic oligonucleotide
  source 1..19: mol_type = other DNA
agcaggaagcagggagaaa

SEQ ID NO: 210 - DNA, 15 bp, synthetic construct
  misc_feature 1..15: note = Synthetic oligonucleotide
  source 1..15: mol_type = other DNA
tccgaccacagcgag

SEQ ID NO: 211 - DNA, 18 bp, synthetic construct
  misc_feature 1..18: note = Synthetic oligonucleotide
  source 1..18: mol_type = other DNA
cagaagccaagggacatg

SEQ ID NO: 212 - DNA, 17 bp, synthetic construct
  misc_feature 1..17: note = Synthetic oligonucleotide
  source 1..17: mol_type = other DNA
cagaagccaagggacat

SEQ ID NO: 213 - DNA, 19 bp, synthetic construct
  misc_feature 1..19: note = Synthetic oligonucleotide
  source 1..19: mol_type = other DNA
ccagaccaacacggaaacg

SEQ ID NO: 214 - DNA, 18 bp, synthetic construct
  misc_feature 1..18: note = Synthetic oligonucleotide
  source 1..18: mol_type = other DNA
ccagaccaacacggaaac

SEQ ID NO: 215 - DNA, 18 bp, synthetic construct
  misc_feature 1..18: note = Synthetic oligonucleotide
  source 1..18: mol_type = other DNA
gaatgggcaaagggtaga

SEQ ID NO: 216 - DNA, 17 bp, synthetic construct
  misc_feature 1..17: note = Synthetic oligonucleotide
  source 1..17: mol_type = other DNA
aatgggcaaagggtaga

SEQ ID NO: 217 - DNA, 17 bp, synthetic construct
  misc_feature 1..17: note = Synthetic oligonucleotide
  source 1..17: mol_type = other DNA
gaatgggcaaagggtag

SEQ ID NO: 218 - DNA, 17 bp, synthetic construct
  misc_feature 1..17: note = Synthetic oligonucleotide
  source 1..17: mol_type = other DNA
gaacccttgtagtcctg

SEQ ID NO: 219 - DNA, 15 bp, synthetic construct
  misc_feature 1..15: note = Synthetic oligonucleotide
  source 1..15: mol_type = other DNA
aacccttgtagtcct

SEQ ID NO: 220 - DNA, 15 bp, synthetic construct
  misc_feature 1..15: note = Synthetic oligonucleotide
  source 1..15: mol_type = other DNA
ggaacccttgtagtc

SEQ ID NO: 221 - DNA, 18 bp, synthetic construct
  misc_feature 1..18: note = Synthetic oligonucleotide
  source 1..18: mol_type = other DNA
atcggaacccttgtagtc

SEQ ID NO: 222 - DNA, 19 bp, synthetic construct
  misc_feature 1..19: note = Synthetic oligonucleotide
  source 1..19: mol_type = other DNA
catcggaacccttgtagtc

SEQ ID NO: 223 - DNA, 18 bp, synthetic construct
  misc_feature 1..18: note = Synthetic oligonucleotide
  source 1..18: mol_type = other DNA
catcggaacccttgtagt

SEQ ID NO: 224 - DNA, 20 bp, synthetic construct
  misc_feature 1..20: note = Synthetic oligonucleotide
  source 1..20: mol_type = other DNA
gatacagacctcctcaaact

SEQ ID NO: 225 - DNA, 19 bp, synthetic construct
  misc_feature 1..19: note = Synthetic oligonucleotide
  source 1..19: mol_type = other DNA
gatacagacctcctcaaac

SEQ ID NO: 226 - DNA, 18 bp, synthetic construct
  misc_feature 1..18: note = Synthetic oligonucleotide
  source 1..18: mol_type = other DNA
gatacagacctcctcaaa

SEQ ID NO: 227 - DNA, 17 bp, synthetic construct
  misc_feature 1..17: note = Synthetic oligonucleotide
  source 1..17: mol_type = other DNA
gatacagacctcctcaa

SEQ ID NO: 228 - DNA, 16 bp, synthetic construct
  misc_feature 1..16: note = Synthetic oligonucleotide
  source 1..16: mol_type = other DNA
gccccatttaccagtg

SEQ ID NO: 229 - DNA, 16 bp, synthetic construct
  misc_feature 1..16: note = Synthetic oligonucleotide
  source 1..16: mol_type = other DNA
cccaacaagtgatgct

SEQ ID NO: 230 - DNA, 15 bp, synthetic construct
  misc_feature 1..15: note = Synthetic oligonucleotide
  source 1..15: mol_type = other DNA
cccaacaagtgatgc

SEQ ID NO: 231 - DNA, 17 bp, synthetic construct
  misc_feature 1..17: note = Synthetic oligonucleotide
  source 1..17: mol_type = other DNA
gtaccaagcccagaagg

SEQ ID NO: 232 - DNA, 16 bp, synthetic construct
  misc_feature 1..16: note = Synthetic oligonucleotide
  source 1..16: mol_type = other DNA
gtaccaagcccagaag

SEQ ID NO: 233 - DNA, 17 bp, synthetic construct
  misc_feature 1..17: note = Synthetic oligonucleotide
  source 1..17: mol_type = other DNA
ttcctgatgaagagatg

SEQ ID NO: 234 - DNA, 16 bp, synthetic construct
  misc_feature 1..16: note = Synthetic oligonucleotide
  source 1..16: mol_type = other DNA
tcctgatgaagagatg

SEQ ID NO: 235 - DNA, 15 bp, synthetic construct
  misc_feature 1..15: note = Synthetic oligonucleotide
  source 1..15: mol_type = other DNA
tgggagtagcatggc

SEQ ID NO: 236 - DNA, 17 bp, synthetic construct
  misc_feature 1..17: note = Synthetic oligonucleotide
  source 1..17: mol_type = other DNA
tgtgggagtagcatggc

SEQ ID NO: 237 - DNA, 16 bp, synthetic construct
  misc_feature 1..16: note = Synthetic oligonucleotide
  source 1..16: mol_type = other DNA
gtgggagtagcatggc

SEQ ID NO: 238 - DNA, 17 bp, synthetic construct
  misc_feature 1..17: note = Synthetic oligonucleotide
  source 1..17: mol_type = other DNA
aaacatgctgaaccctg

SEQ ID NO: 239 - DNA, 18 bp, synthetic construct
  misc_feature 1..18: note = Synthetic oligonucleotide
  source 1..18: mol_type = other DNA
acaaacatgctgaaccct

SEQ ID NO: 240 - DNA, 18 bp, synthetic construct
  misc_feature 1..18: note = Synthetic oligonucleotide
  source 1..18: mol_type = other DNA
cacaaacatgctgaaccc

SEQ ID NO: 241 - DNA, 17 bp, synthetic construct
  misc_feature 1..17: note = Synthetic oligonucleotide
  source 1..17: mol_type = other DNA
cacaaacatgctgaacc

SEQ ID NO: 242 - DNA, 17 bp, synthetic construct
  misc_feature 1..17: note = Synthetic oligonucleotide
  source 1..17: mol_type = other DNA
tggacgcacaaacatgc

SEQ ID NO: 243 - DNA, 18 bp, synthetic construct
  misc_feature 1..18: note = Synthetic oligonucleotide
  source 1..18: mol_type = other DNA
aattttacatactctggt

SEQ ID NO: 244 - DNA, 19 bp, synthetic construct
  misc_feature 1..19: note = Synthetic oligonucleotide
  source 1..19: mol_type = other DNA
aattttacatactctggtc

SEQ ID NO: 245 - DNA, 18 bp, synthetic construct
  misc_feature 1..18: note = Synthetic oligonucleotide
  source 1..18: mol_type = other DNA
ttacatactctggtcaaa

SEQ ID NO: 246 - DNA, 21 bp, synthetic construct
  misc_feature 1..21: note = Synthetic oligonucleotide
  source 1..21: mol_type = other DNA
ctttattataacttgaatctc

SEQ ID NO: 247 - DNA, 35056 bp, Homo sapiens
  source 1..35056: mol_type = other DNA
gcgcaagaatgtaatagagcttcgacggccgccattttctttctttcttagctgttagctgagaggaagtctctgaacaggcggcagcggctcttatagtgcaaccatggcagactattcaacagtgcctcccccctcttctggctcagctggtggcggtggtggcggcggtggtggtggaggagttaacgacgctttcaaagatgcactgcagagagcccggcaggtaagtgtggaccgcgcggcggaatcccgaaagctcacggtaattggccgctgactgagtaggccgctacccttaagcgcatgaggaagaggaaagaggtgttcttccgggctgaaatgtgaagagacacgtttccccatgttggtaataacgattagagaccagaacccagttttgtgttcttggtgcctaatccacttagaaccccgacgcgtgctacgcaaagaaggcctgaagtctttctcccgcttctgcggcactcgcgtgtctccagtgagctagtttagataaagatcctcttccaggggataaagcgcagttagtttcacacaatttaatggaaggttctggtaatgagtttgggaaagaactagggtctgtcctggagccatagcaagggaagggatttatcattaaagtagcctttacagctcatttccgttctctctcgcaattaaaaccgctttcagtaccattcaccgtcacacctctacaaggaagggacttgaaagcagcctttttctgggcgggatttacgtgtcagtctgttccaccagtccgccccccttatttctcaaaatggcctcaggcccattataccagaggtttcaatttgaatctgcctctcagttcagagtcgtaaactgaccagacctctttgtattacgtagtgcgtgcatttgccctgaaggcaccactttcccagacgaaagctgttaaaatagtgcgtgtattccaggaaaataaaagataccttaatttgaactttacattcttagatagtcccctaatatatttaacatttcaaaatgtatggtgttggtatagatttgcatgtaagcaaaagaatcctattctctgtgacactatgcatattgtactaggtgctgggcattttttactagttttaagctaatgataattagaaaccagtgttgtgctgtgttttcgttgcattaggagttcacttagttaactttttaccgggacagttgaaggaacattgagtcaaaattagaattcataaaatccgttgtaacacatctaatgtgaacgcattataaacatgtacctgtacttttttataaccagaaatactaggagtagtcaacaaaaggtcatcattaatattagttctgcggttttttccacgtaatttaagaaattctgaacatgtttagcaacaagcatataactatgacaaacactcttagcgtgttttattagattgatttgtaaaacttaagggaactattttatttactggaaccaactattttattttaccagaaccagcacattggattaattggtatcgcacactgtaggtagatactggagttttgttttgttttgtttgagacggagtctccctctgtcgcccagactggagtgcagtggtgcgatctcgtcccactgcaacctctgcctcccggcttcaagcaattctcctgcctcagcctcccgagtagctgggattacaggcgcccgccaccatgcccggctaattttttgtgttttttttgtttgtttgtttgttttcagtagagacagggtttcaccatgctggccaggctggtctcgaactcctgacctcgtgatccgcccgtcttggcctcccaaagctgggattacaggcgtgagccgctgcgcccggccttgttttttttcgtttgtttgttttaatgcatgaattgtttcctactaagaagctatgatatagttccttgaccaaatgcagatgaacaggattatctgattaatactttaacgagagcagacaaaatatggatatttaattcatccacttgctttataagtgtttatagagtttgttaggaggggatgcccaatttcctaagtaaaggtgatatatgagcaagacattataaaatgaaagagagtttggccagtgaagaaaagaagcagcatgcgcagaggtgtggggacttgagggaatggcaaggacccagaagtgaagtcgctagaagatgggggtgggaggacgtgaaacgaggcaaggaaaggtacaagcggatccagatactggaagaccttgtgttgcaatgctttaattaaaaattggatttcaggccgggtacggtggctcacgcctgtagtcccagcactttgggaggctgaggcaggcagatcacttgaagtcggagtttaagaccagcctggccaacatggtgaaaccccatctttacaaaaaatacaaaaagtagccgggcttggtggcctgtgcctgtagtcctagctacttgggaggctgaggcacaagaaccgcttcaacctgggaagtggaggttgcagtgagtggagatcacgctgctgcactccagcctggtcatagagcgagaccctgtctcaaaaaaaaaaaaattggattttattattaggctaagccataatttctttatacttcttaaaatatagaaattgttggcaaattgtgaaatttattgttgtataatgctaatagatcagttgtcctgatgtctttgctgtaatgatttctttataaaaatgatcttaaatctgagctccctaactttagtttttgcctggaattacccattacatttgatgatatctctaaatgtcagttgtagctgttactctgatgatataagtggaatatacagaagcgtacttagacaaaagttaggttaatatctgaactacttcctccttgtgtatttaagagaatattgacttaagtttctagaatcctcaactaatcctaagtttattttctttgtctagaatactatgctgtttttgtttttggaaggaagagatataggcatagtttcctgctctcaaggagcttcaaaggctgtaccagtggggatgccattggtatttttagctggatagttgttattcagaaaagcaggacaagtaattatgattcctggtccgtacctggtaatgccagtaatgttaactctagctggttgttgacatctggtcatttagttgccaatcttctttttttttttctggcttttatgtgaaatttttagatttttataatatcctgagctaaattcaacacagggacaccagattgctgctttagttcagggtttccagcctgtgcacttaagaaatttatttttgtgtatatcaagctgtaactccagagattgggattgtttgattgggtctttagcagtggtactaatagcaacttctgtctctagaacattggaaaattaaaatgtgtttatctaccgtttttttcctcgaggttatatgaaggtagaaatgaatcagactagatgattagctaagcgagactattaaccctcatcccttcccctctagacaactatgaaattagtcattatgtattcgatccttcttgcagtctcttctctgacagttataaaagtgatttaggctgcataatgttgtttgaatgaaatgaaaatatagactagagctgtttttttttttatttccatcagtctcttcagtgaaaactaacatttgagcatgattctttttttaaatcattttgtgacagtttagcaaggcttgtgataagcaagttatggtatggtaatatttctagtgtccacgtttcttcacatgtctggtgtatgggaactactaactccatcaggaccttgcctatagtaggtactcaacatttactgaattaaatcaataaacatttttaatgaattacagtacaagtcagacctctgtatctgtgggctctgcatctgcaaattcagccaaccatggatcagaaatattagaaaaatggaagaacagtccagcaatacaagtaatatgaataaaaacaatacaacaactatgtacattgtatcaggtattataagtaatttagagatgctttaagtatactgaaggatttgcgtaggttatatgcagatactgtaccattttatataaggaacttgagcatctgtggattttggtatttgcatggttcctggaaccaatcccccagggatactgagggactatagttgatcataccacctgattttagagattttctgagtctcagaagttaattaagtaaactacaatagtctgttcttaacctcggaggatacattccaagaacctcagtgaatatctgaaaccacagatagtattgaatccaatatatacacggtaatattttttcctatacatatgtatctataaagtttaaattctaaatcagacacagtattaacgataataataaattagtgcaagactgggcatagtggttcacacctataatcttaacactttaactatgacgttgtctttgaaaagaaatcagctagccaaggtggctcatggctgtaatcctagtgttttgagaagctaagtcaggaagattgcttgagcccaggagtctgagaccaccctaggcaacatggtgaaaccctgtttctataaaaaataccaaaaaataggctgggcgcggtggctcacgcctgtaatctcagcactttgggaggctgaggcgggtggatcacgaggtcaggagattgagaccgtcctggctaacacggtgaaaccccgtctctactaaaaatacaaaaaattagccgggagtagtgggcgcctgtagtcccagctactcaggagactgaggcaggagaatggcgtgaacccgggaggtggagcttgcagtgaaccgagatcgcgccactgcatgccagcctgggcaacagagcgagactccatctcaaaaaaaaaaaaaaaaaaaccaaaaaattagccagacgtggtggtgcttgcctgtagtaccagctatccagaaggctgaggtgggaggattgcttgaacttgggaggtcaagtctagaatgttgacaatgttgggtcctttatgtagttgcataagtgagccatgatcgtgccactgcactacatccttgggcaacagcctgaccctgtctcaaaattttaatttaattaaaaaaataaaatagaacaattacaacaatacactgtattactggacaagaagggcaaatttaaaaaaaattaaaccaatatgctataataagttatatgaatgaggggaccctcccctaccccagaatatctgattgtactatatcataggtaactgaaaccgtgaagagcaaaaactgaagataaagagactactgtgtcttttaagtttcttttcaactcccaaattcttggatttctcacctcttggcttcctcaatagaggtgagaaatgttaaagtagtgaaaacaggaaaaataacttactcattcaagaagtagataatggtccagatggaaagcttgaattatttttgtaaaactaaaattaaataaagtagccaggcatggtggcttacgcctgtaatcccagcactttgggaggctgaggcgggtggatcacttgcggtcaggagttcaagaccagcctggccaacaaggtgaaaccctgtctatactaaaaatacaaaaattagctgagcatggtggcgggcgcctgtaatcccagctactcgagaggctgaggcaggagaatcgcttgaacttggggggcggacattgcagtgagcccagatcacgccactgcactctagtctgggtaacatcttgagactccatctcaaataataataataattaaataaagtaaaaagtttcccacacctcataaatgtctaataaaaattgaatatgttgagttcaagtactctgaaaaaggagttgaatatagttggaggttggtttttaggaattactatttttcttaaattaactatccttgtagtcacctaggaattgtgtattttctatagatcttagaaaattatcaaatctacagttcattttgttttttcagttttttttttttttttaagagatggagtcttgctgtattagcgttgaactcctggcctcagccagttctcccatctcagcttctgaagtagctggggctgcaggtgccactgagcctggcttctttattggtatttttattaaacacttttctctaatgtctttgtaacagttctcagtttttgaaatgctgttactgtttctttagtgtgaactgtcaactttcattttttcttttcttttcttttcttttctttttttcttttttgagacagagtctcgctctgtcacccagactggagtgcagtggtgcgatcttggctcactgcgacctctgcctcccgggttcaagtgattctcctgcctgagcctcccgagtagctggaattacaggtgcgcaccactgtgcctggctaattttttttttttttttttgtatttttagtagaggtggtgtttcaccatgtcagtcaggctggtcttgaactcctgaactcatgatcccccccgccctgcctcggcctcccaaagtgctgggattacaggcatgagccaccacgcctggcctcagctttcattttcatttggttagtttttgaactattcagtgggtaaagttgtataaataagtgtcttttctctgtatagaagtgtcttggagttcaaggagtgctgctttgcaaactcatagagtatttataaaagctaactgcagaaggtattcataggctaaaccgtttcctattcttggtagcaccattttctctggcctgaaatactttccttctactattagtgcctgtcgatacccagcagtgtatttactttcctgaggaacaattcaaatgctaagtgctttaagacctaagggtggaaaagcagtgttttcaggcattattaggaaaataagatttaaattagacacccagaaacaaaaacaggtttgtaattggtaaagtgaaagatggttaaagaaggttagattgaccaaagcgagaatttaccttttttttttttttttttttgagacagagtctcatgccgttgcccaggctggagtgcagtggcgtgatcttggctcaccgtaacctccacttcctgggttcaagcagttctcccacctgagcctcccgagtagctgggtgacatgcgccaccacgctcagctaatttctttgtatttttagtagagactgggtttctccatgttggtcaggctggtctcgaactcctgacctcagtgatctgcccgccttggcctcccaaaatgctgggattacaggcatgagccactgtgtccggctgagagtgtacctttttttttttttatcaagcaatctagtacttgatcctaataatctttgtggtaggtgtttgcatttttagatgaggaaaagggaaatctatgagtcctaggaaatacagttggtatatgggaactgatatgtaattagacttaagtgatccatgttgaatttatgacttaagcacttaactataatcttaacctctccagttgtctgatgaagttagtatatgggaactgatacatagacttaagtgatccatgttgaatttatgacttcagcacttaactataattttaccctctccagttgtctgatgataataaaaacttgaagcagttatccatatggggatctctttggggaatcccagtcaccaaaagttaggttttctttaatattttttcatggaagatttcaaatatactcaaaattgaaagaattatataataaattctcatgagcccatcacacatcaataatgaatgtacagcattgcagtgtggagcttggcctattgctgaccactcagcaatgtggcagaaccactccatgattccccatggaaatgggaactacttcggttgtccttttatagaaaaattcagtaagtatctgctgattgtgccctacttgtgacttgaagccaggtttttttttttgttttttattttttttgttttgttttgtttgtaacagtcttgctctgtcatccaagaggggcatgatattggtgcactgcaacctccacctcctgggttcaagtgattctcgtgcctcagcctcccgagtagctgggactatgggcgtgcaccaccacacctggctaatttttgtatttagtagagatggagtttcatcatgttgcccaggctgctctcgaactcctgagctcaagcaatctacccacctccacctcccaaagtgctaagattacaggcatgagccaccatgacagcaaagctgggtatttcttaaattggttcagtcaggtgcaataaattatttgccctactctaaaatttaaaaatcttctaagaattatggttttgcagctgaggtttttttaagactcgagctccctggactcctacatatatccttagaacaacattgtccaatagaagtacaatgtgagccacatgttttgtttaacccagcatatccaaaatattaccccctttgcatgtgcttaatataaaaatttaagatgatttatattccaggttttcaatattcagtgagtaattttacacttagagcaagtatcatttcagactagtcacattttgagtactcaataaccacatatggctagtggctaccttactagatagcatagcctttgagtcccacaaagtgttcacattctatgtgtttacacacatcttttgaagtgtcatgacagagccagataggatccagattttctttaaatctggtctctcttccggattcctagttgattacttctttgttgcttcttatagggatgtatcagaagtttaataatcttatgattattatgttaactgcctgaagtattaatggtagcaataaattgaactataattttaatttttcaagtaaattttcttatgtgatactaaatatttcacatatacatgtatcaggaagtaaatgggggtaatttagtaatgaatagtatataatagtttatgatggtatttttcttttttttttttagattgcagcaaaaattggaggtgatgcagggacatcactgaattcaaatgactatggttatgggggacaaaaaagacctttagaagatggaggtaagttatactctaagtattttaaattgtttttcagagtgtttagttgaagtgattctcggtatttttctgttattttattgagatattaacttttattataaggttgttaaaattgtaagctgtatattggcctaaaaggggggaaagaaaactagacaaggtaagtaaaatttgaaagaaattttttaaaaaattttttaaaaaaggaagttttgtcttaatagaaaacaatttattttccctcttttaggattgtgccagattgaaagtttgcacagacgtcttgttaataatattaaaaaacatataaattgcttagaagacacttcactggttttactcatacgtgaatggattttaatatgctgtattttcgtcatttttctatttccaattgcaccttaaaggttgaaattcctatagtttgctactctagtgtgttgcaggttataccattttttttttaatgttctttactttcagtacttttgtgtttcacgtttagctttaaacctgtggattaaaacagtggatttacagtgctatgtatttttaaaaatcgtaatcgttgaagcttctgaacttagaagtctgcatgtattttttgttttaggtttgatatatgagttttgatacattttctttttacccttttttttaaagggaggattctcactgaggctatagaatgtatttgtagcttttgaccaggagaacttggtttccttttatttaagtgtctttcatatttatagtgaggtttttaatgtagaaaaaaaatgagctaatgatgcttcagatgttgtatgtaatgtattctttttattttatgtgtagatggctcttggacaagtccgagcagtacaacacactgggagggaatgccctctccttttaaaggcaggaatttttatttattacctgtgttcagtatgtaaacgtgaaataaaccagtggattcttaaatggacacaaatatttcttggattatgtgtctgcgcatattttatttttgctgcacaacattctgatgtttaatcatttaagtttgaaggggggaggagaatgtagtactttgagctataggttgtctgttccaaggtatgcattgtattcatctgtgtaatggatttaggtgaaggtagtcatgtagttgctttgagattttatttttttgctaaagttttatgcagtgaaatgtttgtttataaataatagaacagtttaggttgagattgccttgtaatgttgtggggggtttttttgtttttttttttttgggcatagctttgtggtcactgtcagatacactttaatatgtcagatttttgtagtttgataggcttttctccccccagtcttcagttcctgaggtggaagcatcattagcctttagcatgtgatattttgctagtaatggacctaaagtacgttgtcttgtgtcattctaatgtgcttaacatacattaaggtcagtgatttcttaagaatcagataactattttaatgtctgtgcatcttttgaacgtgaagagaatgaagtatcgtttcttttttagattactgagttggggttgaattttggcagttttggttcaaatgataaaccataccttcagatatttcaataaatgtttatattgttatttattcttgtttgggaggggagaagctttgtacttaattggcaaaaaattaaaagacacttaattttgcagatcaaccagatgctaagaaagttgctcctcaaaatgactgtaagtattccttttaaactgggtcaaaagctaaagtaacaatttcaatgttaagattttgttcatattattgggtatctttgaagttagttggtttatgagtattttggatcagctagcctgaatttctttgtaaatatatacctttttctcctattttacaatctgtcccattaattgtggccgggtatatgtaaagattggttgctgaattattttacatatttaacaactccaattcttgatctatacttgtacaacttgaaaaaggaaattattttgttctgtgccattgctaatataatgtcttccctttcattggctctcttgcccctgtcaatgccaatataaatattgtgtaaaaaatttgactctcttcaaggtgttgtctgtgcattagggagagatttttgaatgtttgatgtgattgtgttgttaaatatataagtaaatttaaattttgaatttttgttttatttttatttaattttttgatagcttttggaacacagttaccaccgatgcatcagcagcaaaggtatagtcacaagattttcaaaaagtactctgcaagttttggttgagctgtatgtaaaaacacaaccacattggtgtatattgaatatgtgtctgtgtattttttggtgtacctagttcatatcactccccttgggaaggtaccataaagtgatgatttttcttttgagtgagaaaaatttgtgatttggagagataggtggaattaaccacattttagaagaacaggggtgaattagagtaactgttaagatgacattctctaaactccacttcaacttctttacagttaatgccttcagactgttccattcatcatcccttcttcacttgatgtgtcatcttaaatttcttaatttaactactcaagtaataagatcatattttttgacatgagtctgagcctagaaccttagtttaagccattgggagacattagacttccatttttattaatagattatcttttatttgtaaacaaagtatctttcattgaaggaaaatggtgctttctgttatttcttagcagatctgtaatgacagaagaatacaaagttccagatggaatggttggattcagtaagtaacttgatttttaaagttttgaaaacatgatcaaaacatactttagaatctttcaaccaaaaaaaaaatttttttttttctaactagtaattggcagaggaggtgaacagatctcacgcatacaacaggaatctggatgcaaaatacagatagctcctggtaatgttacattctcatggtattttcagtgtgactagaaaactagcttttttttttttttaagccttctagtaacaataatgctacttttaatcttttgacctgaagttatctgtttgttttaaattgtagacagtggtggccttccagaaaggtcctgtatgttaactggaacacctgaatctgtccagtaagtttgaaaaatcttaaaaatctacttaagtaacaacagcagaactctttgaattttgtctcttctctttgttactgctttattttacactgtggtttcgctgccaccttccctcaaagtcctccaactcctttgaagtttatgcctcatgcctttctcaggtggggttcatcatctgaatcattaaacacagaaaatggttaaaacaactccatatctactccagtctctacttgtaaagccacgtgtagcctggagaagaatgcacagtcaggtcgactggtgacacttaaaactcagacattaagctcaagtggactgttgtgttgcctgcatttccctagttccattcacttttccactcctctcccaggctctttaatactgtatttccccacctccaaatcttcagcatctaaccccacgctctcccacttaagcttatttactgagaaaatggaagcaaatgataagaagcttttctttttccctaccactaaacctaccagccttcatttttcctctgttcacatagtactcaggtaattgctttccttttgtgttcgagtgcctaaagccagccctttcttcctactgaaggttcagcttgcagttgtactttcttctgcattgttagttctccctcattactaggttttttcttttcttttttttgagacgaagtctcgctctgttgccaggctggagtgcaggggcacgatctcggctcactgcaacctccgcctcccgagtagctgggactaccggtgcatgccaccacacccagctaatttttgtatttttttagtagagacagggtttcaccatgttggccgggatggtctcaatctcttgacctcgtgatccacccacctcggcctcccaaagtcctgggattacaggcgtgagccacttcccccagcctgattttctttatggcactttccaaataatgtgttattcatttgacatgttatttttacttatttaatgaaatgaagccactctagcaggaaccctgtttctttgagtgctattaccccattacctagaatagcacctgcacatagttgatatttaaatatttgttgaatgaataattgtagcatatgagtaagcaaaatggtagtttaaaaatgtaaataaatcatttagttcttggaagaatcagtttaattctgagataactttagcattagagttctttcttggaaattttggactattcttaaaaataaaaattgtatatctagaaaatttttttgcataatctctcaatctttgacccttgatggcattttctttcagttaaaagtaaaagcattgttaaagttagcatcaaggcacctaatcctgaactgggataggaggagtacttggttatattgttttatatttctctatttgaataagcttgggtatgctacagcttactatttaaatattaatttgttaacaggtcagcaaaacggttactggaccagattgttgaaaaaggaagaccagctcctggcttccatcatggcgatggaccgggaaatgcagttcaagaaatcatgattccagctagcaaggcaggattagtcattggaaaagggggagaaactattaaacagcttcaggtattgttatttttgtgaaatggctacttttgatctgttttgatgcccatttttgtccacttccttttgttaatatatattatttctatgattgtaacaggaacgggctggagttaaaatggttatgattcaagacgggccgcagaacactggtgctgacaaacctcttaggattacaggagacccatataaagttcaagtaaacttaactttatactttataaagaaagagtgggttgaatggggttgggcaaaatatgcatgaataattaaaatgttttgagacatgctttctaaattagctaactttttctgctttagcaagccaaggaaatggtgttagagttaattcgtgatcaaggcggtttcagagaagttcggaatgagtatgggtcaagaataggaggaaatgaagggatagatgtaagtaaaaatacccattcagaaatggttgtatgctaattcataaatataatagtgttttctgttttgtgttaagtagctctaacattgttatccttttatttcacctttatactttagaatacagaattctatatatcttgttaccctatttactataaatatagaattatatgtacttttatgatttgaggcagattttcaggaaatggcgcttttttaaaatacttttttttactttaaaccctgagaagctagctttcttaatacttagtcttttttacataaggtccccattccaagatttgctgttggcattgtaataggaagaaatggagagatgatcaaaaaaatacaaaatgatgctggtgttcgcattcagtttaagccaggtgagtacatataataatcttgtaagtgttggcagcagtgagttttgacatacatttattgtttaattaattttgtttctttgttttgaagatgatgggacaacacccgaaaggatagcacaaataacaggacctccagaccgatgtcaacatgctgcagaaattattacagaccttcttcgaagtgttcaggtttgatagaaagttaacattttcattttttgtttttatggaaaagtattttccttcatgaaatctgaagttacctctatatcagagtctgcttgatgatgctttattaatggagaaagtttaaattgctttaaggtaaagatcttggagcaggaagaactactaccttaagtgctaccttatttactcttgtattttaaaaaatgttattacttattagggccagttcatctctacatttctgattcaggtatatgagagctgggaaaaataaacttaataattttatcatgaaacaaaagtatttctgtgctgactcttcgttcttgtctttccctctctataggctggtaatcctggtggacctggacctggtggtcgaggaagaggtagaggtcaaggcaactggaacatgggaccacctggtggactacaggaatttaattttattgtgccaactgggaaaactggattaataataggaaaaggcaatgtattttaaactcttaatgttttaacacattattcatttttctggacactttctgttgctgtcgtaaacaagtggcaatgctttttctctgaccgtattttagtagaaaagaattcttatgttaatatgtaacaagtaaaacataaatgagggatctcatgtatatttagagaaagagcaggattttaatcttactagcttctagagaaagcgaactaagagataattattagcataagaaatgtcttttgacccaaaaagtggtttgagtgtttttgtttgtgcattttggtttttcccgactcatattttaaaaatttgaatgtttataagtgtattagtttatatttacactgcttttaaaagcagttaattcaaatattttattataatcacattaagtttatgtttaaacatactaagtaaatgtaaatgtattttaagagaagcatgaaatgcttcctaaaatttgattttcagtgtagaatattaaatgaaaaatcttaatacaatattgtcaattaggatactgaccaaaccatatttttaatggcccatttaattgtgaccattttcttctaaatagctcctagtacacccttgaaacctttagagaaattactgtcttttgattttaggaggtgaaaccataaaaagcataagccagcagtctggtgcaagaatagaacttcagagaaatcctccaccaaatgcagatcctaatatgaagttatttacaattcgtggcactccacaacagatagactatgctcggcaactcatagaagaaaagattggtgtgagtatactttaaacttttaatttttagtgtagacccttagactgtagttaaattaagacgtttattcaaatacatcaaaggaaaatgtatcattactagtcagcatttatagatttcatgatatgtataatagatacaacgtgaagattttccagcaatgaaaataacctaattaaatgtgtagttacaggttttgagaacaaccttacatttgggtgtggctagataagaggagggtagtgttacctgtaggcatgatattagtgttggtgtaggattgtggaacatacttgaaggacatagttaacgggaattcattatttattaagattttactctactgaaccccagcgaggcaaacaagataaatcagatacatctgccactctacagtagaaattcataaatcctaggttttggactggctcacagatcatctgggggtattaaaatgtagactatttgggtgcttcccactcctacacataatatacagagacagacctgactcaaaatgtctgggatagggcccagcatctaattttacatagatgtttgggtgattctggtgcacagacaaatttggaaatttttcctccaggaaagttttctgttagaacaaaaaagtatgaaacgctttgactgcttttttgtaagtgaggcagacagtgtcttactggagtttttaacacaaagtgtgcagggggcatcttaaattattagaattgcctaggaaaaattatttttggtgtttgccatgctgtgaatggggtgcgtgcaaaacaaggttgacactgtttctgtcaacttgggaagacaaaaatagatgtacaaaagacgcttaaggaacgtcttacaaaatgtacacaaacattgtgagttctgttgtatggatagtttaatggttcaaaaaatgaagaggatgtttgtagagtaatgatagaggttgatgccatggcaaaaaaatgaatagcactcatcttggttttttattttacagtattagcctaacatgcatgcgtcaggatttctgttggattcatggaaaaacaagatagattgttttggagaagcaatttggtgtggtgattaagagcatggactctgtagtcgggttgcctcagttcagaattctgtgaatcatttactggttctgtgaccttggagaagttactcaggcttttctgtgccttggtttcctcctgtaaaatgaagataatggtatctacttcatagagttgtagggattaaatgaatgttcatgtgtgtgtcacttaaaagaatgcctgccacataaccctaaaaaatgttgctactttttcagtattatttttactacttggaaagaataggtcatggatagtaagtgagagatgacagcaattggagatttaaagagacaagttaataaaaagaactttaaaggtcatgaagtttagtttccctattttgcagtgagaaatttacaacaaaatgtagtgttagcattttgtccaacatgtcgcctggttgtgttaactactcagaaggagcattttaggacagttaagtgtaatgtctttgttggcttaaccaaacagaaatcagttaagcgttattaatgatgtggcatgcatgcatgataaggatataaaatatcctgatttattgaaggaattaaaaagggaatttttgtgctattaaacatcatgatacatgaaaggccaaaaaggatataaattattgatctgaatgggattttagtgacagaaataggttgtgaggtggattttagtttcatagtgaaacaactagctattaacgttatcagtgaaatgttcagaagacgatgatacaggacccaagcggtttgggaatatattgtcaagacggttgtctcatgttaggcaagtagatatagagagaagagctgaagataaggacctgatttctgtgagtaagatggaaaaagatagtaaaagtaagaatgataaaaggaaagggaactactgctgtccgcaaacaagctaaaggaaatttaattgctaatttaaatttaatttaatttaaattgcatttaattgctactgctattgattttagtgaattttacatgtctcattattatggcagatgaaatagtttttgcaaaatgaatgaggaaaggaaggaaaacctaaaatttgttatttgtgactataagagggtagaaatggatgattatttggccgtagagttgtttaaccattactgtgtttttctgatttttctatgtcatccttttttttgtagggcccagtaaatcctttagggccacctgtaccccatgggccccatggtgtcccaggcccccatggacctcctgggcctccagggcctggaactccaatgggaccatacaaccctgcaccttataatcctggaccaccaggcccggctcctcagtaagtattgggtttagttctgggctttccccaaagattctagttttgggactgttttttatgctgatttttcttttcagtggtcctccagccccatatgctccccagggatggggaaatgcatatccacactggcagcagcaggctcctcctgatccaggtagaagatgcttattatttgtgtgttatctgtattattttccactcctgttacattattaaattttctagtgttgattctacatttgtatgcatcaccttcactcactttactctttcaacagtgttaggcactgcctctaccccagtgtataggactgacatgaatatgagctctgcttttatggaatttctttctacttgcctttggcttatgagttgatacagtagaatgataaaagctaaaagctgcaggaaagagcacagtgtcataggttttggataccagtgctgtcaaatgtgtagtatgttcattgtgacattatctgtggaaaaatagtttttacttatttagaaaagtatgtgataggggctgggcactgaggctcacgtctgtaatcccacctattcagaagtctgaggctgaggcttgagaccagcctgggcaatatggcaagacccatctctaaaaaaatttgttttaaattagccgggcagggtggtatgtgcctgtacttgaggctgaggtgggaggattgcttgagcctagaagttctagattgtattgagctgtgggcacaccattgcactcccctgggcaacagaatgggatcccatctcttaaaaaattatgtatatatgtaacagtctatataaatatatatatataacagtccaacagagtgttaagtattggggcattaaacttccaaattgtcaaataagatatctgttctagtactcctcatatgacaacttcatgtgagtaaaaatcaggcctgtatttaataactgcatgctaaagcccaaatacgtttaattattttctatattcagatttatttttatcctattatattctggcatctttccataccctgtagtttgctttccatcttggtcaaagagtcattctttgaaaccaattaacatttatttatgatccttttttctcacctgctacctaccttttaggcctctttcactcgtgtagtttcaaggaaaatatactcaattatggaatactttctacacataatacatttatcccaaaaaaacttgaagtaatttatgtaaatgaaattgcttgattaacttcataggaagtgtgctgtttggaatatgatgacacagcatgacagttaccaagcatgacttgagttgtgctaggatataggtgtgcaaagttggggttgtgttctatagcaaaagaatgcactcccagcgtaagcaacactgatggaaggggctcacagggtacaggatataatactcttaacaactaatttttggagaatgaaagggcttttctttccctcttgttggctgattgggatggtataattaatgggattgaagagtttgagtaggttaaaaggcagattcatattgggtaacttggatctgccaggattgtatttttgagcactactgggtggttagcatgattgaggaaaaatgatgggaataagaagtggaagtggtctttgtatcacaagttgaatttctcacttggagtagtagtgaagtcactactgtaagagctggtcagtgaatgtggttgcagcatggcctttgggcaagaagtaacccatttaactaaaaccagctggttggccccactcagatttatcaaagggttactgggtccctgggggtggatattgcttatattagacttagaatagcatactgttttaatattatatgaactaaaatgtttctttaaaaaaagagtggtctgttaatggatttatgtagtggtcaagaatttagacttcagagtcaaataaacctatatcagtcctagtcctacagtttactaattgtgagatgtcaagcaagtttttgaactcctctaagcctctgttttcttatctataaattaataaatgaatgaatcgggttgagtgaatatttagtaaattcttagtacatactagttatttgtaactgtgagactggttttttggtatggttttcacatttgggagtagaaataccacttcctaaagtctgttttatctcaaattctctatccaggcatagtgtaaagtgaaatacctagatttcttgattaatatacagataatggccagacgccatggctaaaacctgtgacgctagcacttcggaaggctgaggcgggcggatcacttgaggtcaggagttggagaccagcctggcaaacatggcgaaaccctgtctctactaaaaatacaaaaattagctggatgtggtggcaggtgtctgtaatcccagctacttaggaggctgagacaggagaactccttgagaattgctccactgccctccagcctgggcaacagagtgagacacttcatctcaaaaaaaaaaaaaaaatacagataatgacactattgagatatgtaaacatccaacataaaaaagcagtacattgggcaattgagaaaagtttggcaggtgtcctaataacaccttacaattatattaccttgcaattttctttttctagaaattcttgctcatttttctcaaacttgactaacctttattaagcagctgagaattgctactgttcagaatgaaggtataatagaaaatttaaaagttttaattgtatggtattcctagtataaaagacaatcaagttttttctggtttctagaagattgaaaggtctcaaactgttcgcacttcagttgatgtgggagatgagtgagggtcagtcaagtgtagaggagacaacatagctaaaagccgagacacgggcatagtgatttctgaaaagtacaagcactgtgttgtggctggagcttgggtgttgaagagatacaaccaaagggagagatgaggctgaaatggaaaggataagccaggggcttcaggctgttaaagatgttgaatgataggctgtacttaagctttgaacatcctgaagattgcagagaatgaagaatttcaagtaggatgacatcaagtttataacattcaatgtgaaataggatgaaatgtggctaatcttttttaagatttttatattttctcttcattgaaaataaggacaaagttcattgttctaaaataattttctttcttatacaggttagtccctagaaatgtttttctttagtcatcttctagatagagctgtttgtgcttgaggcgaaaccaatttagaaaaaaacaagggcacaggatggtttgagacagagcattggatttggagccaaaagacctttattcatatcccagttatgccactcagtagtttttaacctaggaaagtcacttagtctctgagacttggttttctttaataaaagctgatggtgacaacaaaaataatagaaaatttaagttttcatatactttttttaatgttctagttaattttgggggctgtatatttgccagagagctgggcgggggggtggtgtgtgtgtgtgtgagagagaattacagacttcacatgcaaaccttgaactttcatttattttagctaaggcaggaacggatccaaattcagcagcttgggctgcttattacgctcactattatcaacagcaagcacagccaccaccagcagcccctgcaggtgcaccaactacaactcaaactaatggacaaggtaactacagaacttattgtatgtgaaagccaaaagttgtgcttggaattatatatgaagtacatcactgtataatacctaaaatttctgacattatttaattataatttaagcagacttttccttttttaaattgttactttgaaatagtttcaaactttcataaaagttgtaagaagaatacccggagcacccagatagcccagctgttaatgttttattcccttgtttactctgttaccatctatatcatacatgtgtatctgtatttattttaaatatccattataattagttctgaaggaagctaattatatccagcataattagttctgaaagaaatcactgaagactaaactgtagacatgatgtcctgttgggtacttcttcaaaaatacataaccatcatacagccctccaaatcagtaagtcaacattgatatattaagttctttatatcattatggatttctgatttttgcagtaggttatatttttaaagtatttttataattttgataatcagattatcctggatttggccagtcagggagtatattcagggtggtgcctatatccttttgaaatacctgtcattcttttaagcacttccatactgtctggcacagtaagattgttattttgtgctttctgggctccagccctcgagtcagctatttcttcaaggagctcttattccttttagtagagtatggtagttagaaacgagacttgagtatgcttgttgctactgaggtgtaattgcttctagcttctttcaacaggcagaactaggaaatatatttacatacatgcatacctacatacacacaccaaaaaacacataaacatcgatatatgtatatatttttaaaaactatgttcatatcaccacatccgtttcagcattttggggttttcaagccttttccttttttgtacttgtttacaaacgtgagaaacctggtgtcctcagtgtatttccttatttgacatgcatttacttatatgttcaacataaccagtcttcaaacaggttggttttcttttctgtccaccacctctgtacccccagtaccttctatctttggcactgttagggatgccaccaccacatagtacttccctcctaccctcactgtcacattgcaggcccctgccagctcctgcacccaaggaaacggccaaaattgccttttaaaaactttaattctgttttttgttttgttttgcttgaacaaggagatcagcagaatccagccccagctggacaggttgattataccaaggcttgggaagagtactacaagaaaatgggtatgttttatacatttcttgaaaatacatacttaattaaattgaaacaaattattctcttcaggaagagaataattgaataaaatcactggacttgtaaacatatcaagacagttgtaaattatagtttttaaatttgtggttatatggcaaggaaatttttttttctaattgcatttgtcaaccagttattaattgaaactagaaatgtccttactggtacatacaatattaacattactatacttttgacaatgacagttatatatattatcagtctaaatgacataaggttaaattttaatgtgtcaggcgaaaattgtgtgtgataccattatttttgctgcaagataagcaggtaagaagtaatctgcagtgacggaaagtaaccaagtgatggaaccagaatctggcttccaagagggtctgagtcccaagcttgtctcccaaatttgtctctttaggaaccatttggaacctgatactatacttctggacaaatcactatatttcagctgcttttgctcttagtcatttaaaattattacataccacagctagatgtcacaaatgaaagctaaattggtaagcttggttatccttcactagcagaaaaagaacctataggtggtagagttttgtcataagagaacggtctacttgggattttcaatgttttctttgccaaagaatgtttctcattctccacagaaagaaaaatttccagaaaggtgatgattttaatcttctagatgtaaaattacatatacctgatgataaagttgttttgcacaactggtttctttttaaagaaaaattgtttttcctcttagaatggcttcctaggagagtcatgttccgtctctttctgaggctttaacagattatgtttttgtgacctagcttaggcagatctacagtagctacaattcggcaaaaagaaacttttaacttaaaaacagcatactctgattaaggttggttacataatttattttctgaactgggatcctttttagaatgaatagggatgctattaataatcatgccctgacagcattgtggtcaggacctgtaatcaacttaactttaattaaatagcatcaccattttaaaagacttgagcatgagccacgtgcggtggcacacacttgtagtcccagctgcttgggaggctaaggtggaaggattccttgaacacaggagattgaggccagccttggcatcatagtgagacctggtcacgtgtttgggttaaaaatcactaacttcaacttctattttctcaatgggtaatgtcccctagatagggtccctagttattattaagtaggtaaaaataaaggcttgttaatggatttaggtaattatggaggaatgagtttggcttctgtgctttattttaccatattgattatttgtaatatggccattaatacatttactgtttagtcttttttgttttactttttatgttttactcaaaatgagtgggtgggtggaattctaatttttattgttaagggaagacattttaccttgtctttaattttttatttttttttaccatttccctgccagttagagatactatactatactgtcttgaatcctctgtaggaaaacatggcatagaaataattaaataataattagatgttaaataataatgctgtatgactaaagaacctccttaccccaccttttctgttgttgttctgtagtaccacaataatcacttgttaattttatttatttatttatttgagagggagtctcactctcgcccaggctggagtgcggtggcgcaatctcagctcactgcaacctccgcctcccaggttcaagcgattctccggcctcatactcccaagtagctgggattacaggcgcctgctaccacatccggctgattttttgtatatttagtagatacgggctttcatcatgttggccaagctggtctcaaactcctgacctcaggtgatccacctgcctcagccttccaaagtgttgggattatgggtgtgagccactgcacccagcctcacctgttaattttatgagcaaaacagattagttgggcaagtccttcacatgcatatctcgttgttgttgttttttttaagacggagtcttggtcttactccccaagcaatggtgcgatttcgtctcagtgcaacctctgcttcctgggttcaagctattctcctgcctcaacttcccaagtagctgggattacaggcgcccaaccacacccagctaatttttgtatttttagtagagacagagtttcaccatgttggccaggctggtctcgaactcctgacctcaggtgatccacccgcctcagcctcgcaaagtgttgggattacaggtgtgagccactgcgcctggccacatgtgtatatcttaaaggaaataatgctaggtaatttagtcaggtgcttgatgagcatttgtcatcatcatacggagtcaatttgtctttttctataaaagttctttttgtaaatgattagttgcttaactgctttaatttcttctaggtaccttacctgtcatcaggatctttttaccacaaataagaaaccttaaagcataaaacttggttcagtcttcacatttcttaaatggaaggagaaagggaataatagggagatatctttatctcaaagccaactgttgttgacttttccagtggcaggggtatgatgctaggacttcagatttcctgattccccatcctagtgccccttttgccaaactaggcaggctttcacagcttttggagcctaatttaagtttttcttgttaagaacagaaaactccattcatagattttaatttgtcattatttgcttatttactgaaaaaaaaaaaaaactattgaaaacaggtgtgggttaagtttcccaaaattaggtttatatttcaaaacaattctaagttcccaaggataacaccaaactaagaggataaatttttattttatttttttttattttatttttttgagatggagcctttccctgtcgcccaggctgaagtgcagtggcatgatctcggctcacttgcagcatccacctcccaggttgaagcaattcttctgcctcagcctcccaagtagagaaaatcttgataagatttcagaggcttataaggaacagcagtaaagatgggtgtttttaaagctagaatacagggatttttttaagacccttaagagaacaaacagggtttcaaaatagaaaggagtaattcttgtttggagaagtgaagacatttgggaaaagtttgacttgaggaaggccattatgataaggactgatgtggtagactctgaaccctgaagaatctggtaccttaagcctaaaggagggtagagtgaggacttgtgggaaaggtacagttatatgggaagacaactgagtgtgaaaatctattcatgcaaggctggctcttaggctctaatagaatcttgcaagctaactctagagcaagtcttttatctttgggatgaaagaaatttaaggctaaactttatacattataattataaaactataataaatattagataatgttctactgaagtataaactcaactatctggcatcaaacagtaactaagccatgatcacaccactgccactgtgttctggcctaggtgacagagcaagaccctgtctcaaaacaaaatccagtaactaaagagaggaataaggggagcgcaaggtaaggcagtacatgtctgaaggggcagggaaagagttctttctctacttccaactgggcaaaataaatcacatttgccctttagattggaagagtgaaataagcttttcttgaacatcttttaagattggagtgtcaaatattaccaacttatttaccaagaatttggtttttcttaaagtctaaagtgtttactattagctttccacagggatataggagtttgcagaggttgtagttttttaagggaacatcaatgaattttcttgatgcatatgcctgtttctaccattttacattgttaatttgcctctaaaatgagtaactcttacaatggggtttaaaacctgaagactattgattgctaccttgatcaggtttggtttcaagtgtgcaccctgtaagagacaatgtgtggttttattgctattgtcacaccagttttttagatattgaaacctgtttcagatttgcttgaattgtgtcttgtaagaggaaaatgttaaacttacttcctctttgagaacagttatttatagaagacaggaaaatatgagaatttttaatagtatatgagagttctctgttacccaagaaaagagggtttttttcaggcatttttaaagaatcataaatcttaaattctttcactcagttgctttgagtctgtgacctgttttacaatggtgatagactgctttctgaaactatgaaattggtcttgttggcagcatcctacaaacataaaaagagcttcctgtgtgtccatgctccagtatttttgtctatggtagttatttcacaaagccaagcgagttacaaacgaaataaaatagtgcttaagtaaagaaactgaataggagaacatatactctctcttctcaattttttatttagaaaattatataccttcagaaattggggaaataaggcatcagacactcagcttcatcattttttaaaagtttgctgtatgcgctttatatgtatgttttttgtgtatgaaccatttcaaagtaagttgcagacaagacagtttgctctaaataaactcaatgtctgggcacagtggttgacgcctgtaatcccaggactttgggaggccaaggcaggtggatcacttgagtccaggagttcaggaccagcctggacaacatggcaaaaccccatctctacaaaaaaagagaaaaatgtcagctaggcatggtgggcctgtagtcccagctactctagaggctgaggtgggaggatcacctgagccctgggaggttgaggctgcagtgagccatgattgcaccgctgcactccagcctgggtggcagagtgagaccctgtctgaaaataaataccctcaatattatcacaaacatacagactatatttaaatttccatagttgtcaggaatatgtcttttgtcgtctgtgtgttttgtttaattcagagtacattcaagattcatgcgttgcatttcattgctatatttctttaatctcttaattcagcaaagtcaccttatttggaaaaagacctgattgtgaaagctgagttttatgtgttttcattgatcttgttctttattccttgtcaggttacttttaataacattgtgttgtaattggaataaattattaatacttttaacatcttattatgtgtgtgtgtacatacactttataggttactaatcaggaaaaagtcttggctaggtcttaatacagtctttaaatcattgcctttaagtgggcttaaagtttttcaaaaatgttctttttgtaattctggaatcgaattaagattatgcctaaatctttaccttccttagctaaagcagtgtggatttggggttgattctgtttttttactaataatgacgctctagaactaaaagttaacgattaattataaggcaaaaagaaaagagcgttctttttttttttctaattgcagagtagtttcctgacactactaaatgaatattttaaataaaacaggagtaattctgaccctctgtgcttttgtcttataacctgtacttacagtggatgtaattttatattaaagtttaggggtttttttttttggtcaacaagggcaaacacaagattattcaaaggcttgggagaaatattacaagaagcaaggtattgtttttattagaaatgagatgttgggcttataattgtggttacagcaacaaagttcttttttttcaaaggtcaggcagttcctgctccgactggggctcctccaggtggtcagccagattatagtgcagcctgggctgagtattatagacaacaagcagcctattatgcccagacaagtccccagggaatgccacagcatcctccagcacctcaggtataatgtaattgctaatttgttgatttctactccagtctgttttctgcatgtttactgtttgtctgtttgggagtgtttgccttttaaatttttatctggcaaagtataataactatttaaatgaagtactacggtgtattgtttgggtttttttgttttttataatgctttccagcatctgagtggtgaatatttctgcaatgcctttgattttaaaaataaattttcttcccccagggatttgcaaatcatgcaagaagccaccaccatttatattaaccactttttctttcttaaaggattcactcctgaattagctccatttcaaggattttctttaactttttgtgtatttcttatgtatctcttctgcacagggccaataataagaagtggacaatacagtatttgcttcattgtgtgggggaaaaaaacctttgttaaatatatggatgcagacgacttgatgaagatcttaattttgtttttggtttaaaatagtgtttccttttttttttttttttttgaaaatgtacaaaatatctatcactactgataggaggttaatatttctgtgtagaaatgaaaattggtttgtttttagtatttagtgtagatgtacacattccagcaaatgtatttgcaattatgtggttgatgctttgtgatataaatgtactttttcaatgtatactttcacttttaaaatgcctgttttgtgctttacaataaatgatatgaaacctcctgtgtcggtaagttggatatgtgggtatttaaaggattcataatttcttagcaatgataaattaagatacatatacacaaatatataagctttccccatgaaatattgagtttttaaacactggcatgtttttccccccttgcagtatagtggtagattggaggatcttttccatttattgtattggctctttcagcacaagtaatcctgatatcttcattttttttccttctgtttgattaaaaactgcatgtgtgtacaatgatcttttggcatacttccattgcattaacagtgaaatttccttttatacatgaccactgtttcagacctgtactgctgctataacagttaacctttctgttcttaatttgataatacttgatttccaagactgtttcggcataactaattttaaacagttttcagatagtgaatatgagtagtctaataagaacagtttttttccatgtgaagcaactctttcaatgtatataatgttagtgtgtttctttctaaatttaggatagaaaagtgaatagtgtgcaaaaagtatagctacattgcatctgccattgaaacataaatggggtatggaaacgttcaagctttttttttttctttatgcagtatagataagctttgttttgtaaatgcacaagtccaatcattgaatcaacttaatttttttatgtacttgaagtcattttattactctttaacactcatgctgaagttctgatattttgttgaaatccattgttttactctttgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagaatgacaaattgatgaactgcaacagtatctttttgtcaatgttccacataatgcaaatgccatacgttgtgtgaatattatgttggaatacagtgctgatatcttggaaaaccataactgcctcttaatttaacatagaataatacatagttctgtattttttttaaagtgagcttaatgggtaagtattttttatatgctttagctatagctaaagaaaactgatacttaacaaagttgaatagtattattcactggtgctcctaaaatattgtttttcagtgtaaaatatgcatatcttctatatttaatatgaaagtcttgaaatgtatcagacagaaggggatttcagtttgcaaataatgagcaatgtagcaattttaacacatttcataaatatatattttgtcattggtggagagcaccatttgttgttttgaatatactttaaaggaagaggtacaaggacataaatgttgagattacctacaggatggaaatagcagtacagttcattgtagatattttgaaatgtttttgattgttttatataacctagagtgacttcccttacccttatttagatctgcatatatagttctagtatgaagtttaatagttaaggagttagctatttgttatctttaagagtagggtattgacgtgaacaattgcagtattttgcatgatactgttttatagatgaccttttaggaaagtggtgcatttattaattgaactgaagaagtagttcagttgaattcagtatcataattcacaaattggaggctgttgattttgattcatttaaggtttaaaatctttattaattgcaaacagtgcaattatttatacttcacagtgccttcccagaccttccaccttaggttctgctgcaaaaagcaccaggtaagcacaacctaaggacatatataaataaatatttcaatacattaatgttgtccctgtgaggtttttgtggttgtgtattcaaaggcaatctgctactgcttccccaaaatgtattttgttattttatgctaccatcttagtggaaagtctgtaagttgttaaagcaactgtttacatttctgggtaatgttttttattttacttttttttttttattaagacaagaaaatgatgagtagattgctgcagtaattgaactacatccaaatctttttgtattttttccccaaatatagaagtgttaatattaagaaaggacaattacacagttttcaagatttaggaaatcacttgtttagaaacttcaacagccttcacaatctgttttatatgatggacagaaaatttctttgccctccaaaattataatttctttatttttttcttattcttaaactataataattcagtaaggatattatgggttagaattttattatgatttttttcttagacaaaagttatatgctgaagaaggaaaaagttataaggcagtatgttttgataaaaggcatgtgcatcagtgaaatgttaactgtatagcaaataacctttcataatctgtagcaatcagtatttttctgatttaataatattttaataactgacgctgcatttaatttttttgccagtttaaaatgtttgtgtgtttttatagatgattttaactggtacatattttgagttaagttgaatgtatgaaagcagcatcttatcagttttgtttattcgatttctaaaatgtgctgatccttttaaaactcctgcttatctctgcaacaaagaaaaatattcaaaaatactgccttcattttcacacacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgaaatgtattcccatacataatatggtatagggtgtaatgtacctgcttttgatcacttttcattttaaagtgctattcacttgatcttaaatgttccatgaactgttaaatttcttaagttacatagttactacaccacatttatgtgtatgttatgttttaatagtcaatgataggtatgtacaattgataatataaaggggctcattgaaacttgagagcctgttgagttttggttagttgtagattgcatttttataaaaaaaaatacagatagattgatgataatagatattggggcattgtttctgtctcatgagaattcttttattcattaccataagccttcactgatactataagcattattttaaatgacgctgatcttaagtctgaaataaatggaaagcagaaaaggtgagccagttgatttgaatgcattggatattagtgttagaaacaatgtatagtttagattgaaactgaactgacttatttagcacttaaacaaaaatttgacaatgtttttagttttttttaagacagcttagtgtggtgatacttagaattctatggtttgatgtttcttttagaaatgagaagtatagttttattttttaatataaaaaatggttttaatactaaaactagtaatttgatactagttgtttataaacattgtaaaatatatcttttaaacaaattatcttggtagttaattcataagggtgggtttgggtaggaatagcagagtactttcagagggaaaggggagtcattcagaagtgatagcattttatttgtttgaatactctgccagtaaaatcagctgtacttagaaagttatctgttgtgtagaataatgatgtagagtttactaatcagtgaggatgtcttgtttttattttctgcaaactctgcctcactttaaaatgcattataacaatacctaattaaagataattttggctctgaaagttaccttattttttgttgagttagtgacttcatttttcttgccacaatataagcttttgagggatttttttaaattggtgcttttaataagcaaataaatcccagggttttattttcttcagtgatacccctatagaaactcttaaatgtatttgcgcatatatatatatatattttcttatgcatgctcgatgcattttcgtcctgagaaaaatgttctctacagaaactacccgtgtgtaaaaagaagattggcttaaaatggctactgtgatgggaacagtgtcttagggagatgcagcttggacttgaggtaaattgaatactttacaactgtggtttagagtttgctttaatgacattgtatgtaaaaggtcacatgattgctgtaattttgtattcattatggtttcctcaataaatgtacattgatgactatta

SEQ ID NO: 248 - DNA, 696 bp, Homo sapiens
  source 1..696: mol_type = other DNA
atggcagactattcaacagtgcctcccccctcttctggctcagctggtggcggtggtggcggcggtggtggtggaggagttaacgacgctttcaaagatgcactgcagagagcccggcagattgcagcaaaaattggaggtgatgcagggacatcactgaattcaaatgactatggttatgggggacaaaaaagacctttagaagatggagatggctcttggacaagtccgagcagtacaacacactgggagggaatgccctctccttttaaagatcaaccagatgctaagaaagttgctcctcaaaatgactcttttggaacacagttaccaccgatgcatcagcagcaaagatctgtaatgacagaagaatacaaagttccagatggaatggttggattcataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattccagctagcaaggcaggattagtcattggaaaagggggagaaactattaaacagcttcag

SEQ ID NO: 249 - DNA, 1968 bp, Homo sapiens
  source 1..1968: mol_type = other DNA
atggcagactattcaacagtgcctcccccctcttctggctcagctggtggcggtggtggcggcggtggtggtggaggagttaacgacgctttcaaagatgcactgcagagagcccggcagattgcagcaaaaattggaggtgatgcagggacatcactgaattcaaatgactatggttatgggggacaaaaaagacctttagaagatggagatcaaccagatgctaagaaagttgctcctcaaaatgactcttttggaacacagttaccaccgatgcatcagcagcaaagcagatctgtaatgacagaagaatacaaagttccagatggaatggttggattcataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattccagctagcaaggcaggattagtcattggaaaagggggagaaactattaaacagcttcaggaacgggctggagttaaaatggttatgattcaagacgggccgcagaacactggtgctgacaaacctcttaggattacaggagacccatataaagttcaacaagccaaggaaatggtgttagagttaattcgtgatcaaggcggtttcagagaagttcggaatgagtatgggtcaagaataggaggaaatgaagggatagatgtccccattccaagatttgctgttggcattgtaataggaagaaatggagagatgatcaaaaaaatacaaaatgatgctggtgttcgcattcagtttaagccagatgatgggacaacacccgaaaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccagcagtctggtgcaagaatagaacttcagagaaatcctccaccaaatgcagatcctaatatgaagttatttacaattcgtggcactccacaacagatagactatgctcggcaactcatagaagaaaagattggtggcccagtaaatcctttagggccacctgtaccccatgggccccatggtgtcccaggcccccatggacctcctgggcctccagggcctggaactccaatgggaccatacaaccctgcaccttataatcctggaccaccaggcccggctcctcatggtcctccagccccatatgctccccagggatggggaaatgcatatccacactggcagcagcaggctcctcctgatccagctaaggcaggaacggatccaaattcagcagcttgggctgcttattacgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaggcagttcctgctccgactggggctcctccaggtggtcagccagattatagtgcagcctgggctgagtattatagacaacaagcagcctattatgcccagacaagtccccagggaatgccacagcatcctccagcacctcagggatttgcaaatcatgcaagaagccaccaccatttatattaa

SEQ ID NO: 250 - DNA, 1935 bp, Homo sapiens
  source 1..1935: mol_type = other DNA
atggcagactattcaacagtgcctcccccctcttctggctcagctggtggcggtggtggcggcggtggtggtggaggagttaacgacgctttcaaagatgcactgcagagagcccggcagattgcagcaaaaattggaggtgatgcagggacatcactgaattcaaatgactatggttatgggggacaaaaaagacctttagaagatggagatcaaccagatgctaagaaagttgctcctcaaaatgactcttttggaacacagttaccaccgatgcatcagcagcaaagcagatctgtaatgacagaagaatacaaagttccagatggaatggttggattcataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattccagctagcaaggcaggattagtcattggaaaagggggagaaactattaaacagcttcaggaacgggctggagttaaaatggttatgattcaagacgggccgcagaacactggtgctgacaaacctcttaggattacaggagacccatataaagttcaacaagccaaggaaatggtgttagagttaattcgtgatcaaggcggtttcagagaagttcggaatgagtatgggtcaagaataggaggaaatgaagggatagatgtccccattccaagatttgctgttggcattgtaataggaagaaatggagagatgatcaaaaaaatacaaaatgatgctggtgttcgcattcagtttaagccagatgatgggacaacacccgaaaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccagcagtctggtgcaagaatagaacttcagagaaatcctccaccaaatgcagatcctaatatgaagttatttacaattcgtggcactccacaacagatagactatgctcggcaactcatagaagaaaagattggtggcccagtaaatcctttagggccacctgtaccccatgggccccatggtgtcccaggcccccatggacctcctgggcctccagggcctggaactccaatgggaccatacaaccctgcaccttataatcctggaccaccaggcccggctcctcatggtcctccagccccatatgctccccagggatggggaaatgcatatccacactggcagcagcaggctcctcctgatccagctaaggcaggaacggatccaaattcagcagcttgggctgcttattacgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaggcagttcctgctccgactggggctcctccaggtggtcagccagattatagtgcagcctgggctgagtattatagacaacaagcagcctattatgcccagacaagtccccagggaatgccacagcatcctccagcacctcagggccaataa

SEQ ID NO: 251 - DNA, 39750 bp, Macaca fascicularis
  misc_feature 1246..1266: note = n is a, c, g, or t
  misc_feature 2077..2131: note = n is a, c, g, or t
  misc_feature 2666..2690: note = n is a, c, g, or t
  misc_feature 3304..3345: note = n is a, c, g, or t
  misc_feature 3869..3870: note = n is a, c, g, or t
  misc_feature 6076: note = n is a, c, g, or t
  misc_feature 6091..6093: note = n is a, c, g, or t
  misc_feature 6159..6183: note = n is a, c, g, or t
  misc_feature 6259..6277: note = n is a, c, g, or t
  misc_feature 6869..6906: note = n is a, c, g, or t
  misc_feature 7072..7139: note = n is a, c, g, or t
  misc_feature 7714: note = n is a, c, g, or t
  misc_feature 11374..11394: note = n is a, c, g, or t
  misc_feature 11454..11491: note = n is a, c, g, or t
  misc_feature 12562: note = n is a, c, g, or t
  misc_feature 12699..12742: note = n is a, c, g, or t
  misc_feature 12793..12794: note = n is a, c, g, or t
  misc_feature 13648..13656: note = n is a, c, g, or t
  misc_feature 14247..14261: note = n is a, c, g, or t
  misc_feature 14474..20234: note = n is a, c, g, or t
  misc_feature 21758..21789: note = n is a, c, g, or t
  misc_feature 23223..23225: note = n is a, c, g, or t
  misc_feature 24454..24512: note = n is a, c, g, or t
  misc_feature 24563..24589: note = n is a, c, g, or t
  misc_feature 25270..25338: note = n is a, c, g, or t
  misc_feature 25723..25734: note = n is a, c, g, or t
  misc_feature 25857: note = n is a, c, g, or t
  misc_feature 26517..26523: note = n is a, c, g, or t
  misc_feature 27589..27590: note = n is a, c, g, or t
  misc_feature 27660..27688: note = n is a, c, g, or t
  misc_feature 28733: note = n is a, c, g, or t
  misc_feature 30266: note = n is a, c, g, or t
  misc_feature 30317..30371: note = n is a, c, g, or t
  misc_feature 30453..30768: note = n is a, c, g, or t
  misc_feature 30833..30837: note = n is a, c, g, or t
  misc_feature 30906..30912: note = n is a, c, g, or t
  misc_feature 30997..30998: note = n is a, c, g, or t
  misc_feature 31271..31562: note = n is a, c, g, or t
  misc_feature 31697: note = n is a, c, g, or t
  misc_feature 31707..31782: note = n is a, c, g, or t
  misc_feature 32257: note = n is a, c, g, or t
  misc_feature 33477..33478: note = n is a, c, g, or t
  misc_feature 37353..37427: note = n is a, c, g, or t
  source 1..39750: mol_type = other DNA
gagatttcgttccttcagtctccacccctttacggcacgatggtcgcgcaagaatgtgatacagcttcgacggccgccattttctttctttcttagctgttagctgagaggaagtctctgaacgggcggcagcggctagcgtagtgtaaccatggcagactattcaacagtgcctccaccgtcttctggctcagctggtggcggcggtggcggcggtggtggtggaggagttaacgacgctttcaaagatgcactacagagagcccggcaggtaagtgtggaccgcgcggcggaatcccgaaagctcacggtaattggccgctgactgagtaggccgctaccctttagcgcatgaggaagaggaaagaggtgtccttccgggctgaaatgtgaggagacacgtttccccttgttggtaataaagattagagaccagaactcagttttgtgttcttggtgtgtaatccacttagaaccccgacgcgtgctacgcaaaggcctgaagtctttctcccgcttctgcggcactcgtgtgtcgccagcgagctagcttagcctccccttttcctcgagatgaagatcctcttccaggggataaagcgcaggtagtttcacacaatttaatggaaggttctggtaatcagtttgggaaagaactagggtcggtctcctggagccatagcaagggaagggatttgtcgttaaagtagcctttacagctcatttccgttccctctcgcaattaaaaccgctttccgtacctttcaccttctcacctctacaaggaagggacttgaaagccgtctttttctgggcgggatttacgcgtcagtctgttctaacagtcagtcccccttatttctcaaaatggcctcaggcccattataccagaggtttcaatttgaatctgcctctctgttaagagtcgtaaactgaccagacctctttgtattacgtagtgcgtacatttgccctgaagacaccactttcccagacgaaagctgttaaaatagtgcgagtattccaggaaaatagaagatttcttaatttgaactttacatttttagatagtcccctaatatatttaaaatttcaaaatgtatggtgttggtatggatttgcatgtaagcaaaagaattctattctctatgacactatgcatgttgtactaggtgctggacatttttactagtgttaagctaatggtaggtagaaaccagtgttgtgctgtgttttcattgcattnnnnnnnnnnnnnnnnnnnnncggtacagttaaaggaacattgagtcaaaatcagaattcataaaatccgttgtaacatacctaatgtgaacacattatcaacatgtacctgtacttgtttataaccagagatattagaggtagtcaacaaaaggttatcattaatattagttctgcggttttttcacataatttaagaaattctgaacatgtttagcagcaagcatttgtataactatgacaaacactgttagcgcgttttattagattgatttgtaaaacttaactattgtatttattggaaccaactattttattttactagaaccagcacattggattacttagtatcaaacactgtaggtagatactggagttttgttttgttttgtttgagacggagtctccctctgtcgcccaggctggattggagtagcgtggtgcgatctcgtcccactgcaacctccgcctccggcttcaagcaattctcctgcctcagcctcccgagtggctggaattacaggcgcccgccaccgtgcccggctaattttttgttttgtttttgttttttaagtagagacagggtttcaccatgctggccaggctggtctcgaactcctgacctcgtgatccgcccgccttgacctcccaaagtgctgcgattacagcagtgagcccggccttttttgttgttgttgtttaaatgcatgaattgtttcctactaagaagctatgatatagttccttgaccaaatgcagatgaacaggattatctgataaattgattaacgagagcagacaaaatacgggtatttaattcatctgctcattnnnnnnnnnnnnnnnnnnnnnnnnnnnnnnnnnnnnnnnnnnnnnnnnnnnnnnngttgggaggggatacccagcttcctaagtaaaggagtgatatttgagcaaggcattatagaataaatgagagtttggccagtagggaaaagaagcagcatgcggagaggtatgggacgtgggggaatgacaagaacccagaattgaagtcactagaagatggtggtgggaggacgtgaaacaaggcaaggaaaggtacaagcggatccagatactggaagactttgtgttgcaatgctttaatgaaaaattggatttcgggccgggttcggtggctcatgcctgtaatcccagcactttgggaggctgaggcagacagatcacttgaagtcggggtttaagaccagcctggccaacatggtgaaaccccatctctacaaaaaatacaaaaggtagccgggcttggtggcctgtgcctgtagttccagctacttgggaggctgaggcacaagaattgcctgaacctggaaagtggaggtagcagtgagtggagatcatgctgctgcactccagcctgggcatagagcgagaccctnnnnnnnnnnnnnnnnnnnnnnnnnggttttattattaggctaagccataatttctttctactttttaaaatacagaaattgccatcaaactgaaatttattgtcttatatgctaataggtcagttgttctgtctttactgtaatgatttctttatgaaaatgatcttaaatctgagattcctaactttggcttttaactagaattacccattacatttgatgatgtctttaaatgtcagttgtagctattagtctgataatatgtgtggaatatacagaagggtatttagacagaagttaggttaacatctgaactacttcctccttgcgtatttaagagaatattgagttaggtttctagaatcctcaactaactctaagtttattttctttgtctagaatactatgctgtttttgtttttggaaggaagagatataagaacagtttgctgctctcaaggagcttcaaaggctgtaccagtggggatgccattggtatatttagctggatagttgttattcagaaaagcaggacaagtaattttgattcctggtccgtacttggtaatgtcagtaatgttaactctagctggttgttgacatctggtcatttagttgccaattcctttttttttttnnnnnnnnnnnnnnnnnnnnnnnnnnnnnnnnnnnnnnnnnntattctgagctcaattcaacacagggacaccaggttgctgcttttgttcatggtttccagcctgtgcacttaagaaatttatttttatgtatttcaagctgtaactccagagattgggattgtttgattgcgtccttagcagtgatactaatagcaacttctgtctctagaacattggaaaattaaaatgtatttatctaccgttttttcccttgaggttatatgaaggtagaaatgaatcagactagatgattagctaagcaaaactgttaaccctcatcccttcccctctagacaactatgaaattagtcagtatgtattcgatccttcttgcaatctcttctctgacaattataaaagtgattcaggctgcataatgttgtttgaatgaaatgaaaatacagactagagctgtttttggttttgtttttttttttatttaccatcagtctgttcagtgaaaactaacatttaagcatgattctttttaaaaatcattttgtgacagtttagcagggcttgnntgataagcaaactatggtatggtaatatttctagtgtgcacgtttcttcacatgtctggtatatgggaactctaactccatcaggactttgcctatagtaggtactcagcatttactgaattaaatcaataaacatttttgatgaattaaagtacaagtcagacctctgtgtctgtgggctctgcatctgcaaattcagccaactgtggatcaaaaatattagaaaaatggaatgacggtccaacaatacaagtaatacgaatgaaaacaatacaactatgtacattgtatcaggtattataagtaatttagagatgctttaagtataccaaaggatttgcataggttgtatgcagatactgtaccattttgtgtaaggaacttgagcatctgtggattttggtatttgcatggttcctggaaccaatccctcagggacactgagggactatagttggtcataccacctgattttagagattttctgatcctcagaagttaattaagtaaactacagtagtctgttcttaacctcggaggatacattccaagaacctcagtgaatatctgaaaccacatatagtattgaatccgatatatacacggtaatattttttcctatacatgtgtatctataaagtttaaattctaaattagacatagtattaacaataataataaattagaaaaagactgggcatagtggttcacgcctataatcttaacactttaacttttaactatgacgttgtcttgaaaaagaaatcagccagccaaggtggctcatgtctgtaatcctagtgttttgagaagctcagtcaggaagtttgtttgaacccaggaatccgagaccaccctaggcaacatggtgaaaccttgtttctataaaaaaaaaccaaaaaattagccagacctggtggtgcatgcctgtagtaccagctacccaggaggctgaggtgggaggagtgcttgaacttgggaggtcaagtttacaatgttgacaatgttgggtcctttacgtagttgtgtaagtgagccatgatcatgccactgcactacagccttgggcaacagcctgaccctgtctcaaaattttaatttaattttaaaaatgaaatagaacaattacaacaatacgctgtattactgacaagaagggcaaatttttaaaaaaccaatatgctgtaataagttatatgaataaggggatcctcccctaccccagaatatctgattgtactataccgtaggtaactgcaaccgtggagagcaaaaactgaagatactgtgtcttaagtttctttttcaactcccaaattcttggatttctcacgtcttggcttcctcagtagaggtgagaaatgctaaaacagtgaaaacaggaaaaataacttactcattcaagaagtcgattatggtccagatggaaaatttgaattatttttgtaaaactaaactaaagtagccaggcaccgtggctcacgcctgtaatctcagcactttgggaggctgaggcgggtggatcacttgaggtcaggaattcaagaccagcctggccaacaaggtgaaaccctgtctctactaaaaatacaaaaattagtcaggcatggtggcgggcacctgtagtcccggctacttgggaggctgaggtgggagaatcgcttgaacctggggtgtggacattgcagtgagcccagatcacgccactgcactccagcctgggcaacattgtgagactacaaaaaatataatagtaagtaaagtaaaaagtttcccatacttgataaatgtctaataaaaattgaatatgttctaggactctgaaaaaggagttgaatatagttggaggttggtttttaggaattatttttcttaaattaattatccttgtagtcacctaggaattgtatattttctgttgatcttagaaaattgatcaaatctatagttcattttgttttttcaatttttttttaaagagatggggtcttactgtattaatgttgaactcctggcctcaaccaggtctcccacctcagcttctgaagtaactgggattgcaggtgccactgagccaggctcctttattggtatttttattaaaagcttttctctaatgtctttgtaacagttctcaatttttgaaatggtgttactcattctttagagtaaactgtcaactttcattttcttttttttttttttnttttttctttctttnnnttgagacggaatctcgctctgtcacccaggctggagtgtagtggtgggatctcggctcactgtgannnnnnnnnnnnnnnnnnnnnnnnntctcctgcctcagcctccagagtagctggaattaacgggcacgtgacaccgtgcccagctactttttttttttttnnnnnnnnnnnnnnnnnnngatgggggtttcaccatgttggtcaggctggtctcgaactccagacgtcaggtgatccacccacctcagcctcccaaagtgctggtattacaggcatgagcaacagcgcccagcctcaactttcattttcatttggttagtttttgaactattcagtgggtaaggttgtataaatgtcttttctctgtatagaagtttcttggagttcaaggagtgctactttgcaaactcatagagtatttataaaagctaactgcagaaggtgttcataggctaaaccgtttcctattcttggtagcaccattttctctggcctgaaatactttccctctactattagtgtctgtcagtgcccagcagtgtatttactttcctgaggaacaattcaaatgctaagtgctttaagacctaagggtggaaaagcagtgttttcaggcattattaggaaaataagattttaattagacacccagaaacaaaaacaggtttgtaattggtaaagtgaaagatggttaaagaaggttagattgaccaaagctagagtttttctttttttctttttttttttttttttttttgagacagtgtctcnnnnnnnnnnnnnnnnnnnnnnnnnnnnnnnnnnnnnnggctagctgcaacctccacttcctgggttcaaacagttctcccacctgagcctcctgagtagctgcgaggcatgtgtcaccatgctcagccattttttgtgtgtttttagtagagactgagtttctccatgttggtcaggctggtctcgaactcttgagctcaggnnnnnnnnnnnnnnnnnnnnnnnnnnnnnnnnnnnnnnnnnnnnnnnnnnnnnnnnnnnnnnnnnnnntgtacctttttttaatcgagcaatctagttcttgatcctaatagtctttgtggtgggtgtttgcatttttagatgaggaaaagggaaatctaggagtcctaggaaatacagttggtatatgggaactgatacgtaattagacttaagcaatccatgttgaatttgtgacttaagcacttaactataaatttaccctctccagttgtctgatgataatcaaaacttgaagcagttatccatattgggatctctttggggaatcccagtcaccaaaagttaggttttctttaatattttttcatggaagttttcaaatatactcaaaattgaaagaattatgtaataaattctcatgagcccatcacacatcttcaataatgaatgtacagcattgcagtgtggagcttggcctattgctgaccactcatcaatgtggcagaaccactccatggttccccatggaaatgggagctacttcagttctctttttacagaaaaattcaataaatatctactgattgtgccctacttgtgacttgaagccaggtttgtttgttgttgtttttctttttttnttttttttttttttttactagtcttgctctgtcacccgagctacagtgcaatggcatgatatcagtgcactgcaacctccagctcctgggttcaagtgattctcttgcctcagcctcccgagtagctgggactatgggcgtgcaccacaatacctagctaatttttgtatatagtagagatagagcttcatcatgttgcccaggctgctctcgaactcctgagctcaagcagtctacccacctccacctcccaaagtactaagattacaggcatgagccaccatgccagcaaagctgggtatttcttaaatttgttcagtcaggtgcaagaaattatttgccctactctgaaagttaaaaatattctaagagaattatggtttcgcagctggggttttttaagacttgagctcctggggctcctgcatatatccttagaacaacattgtccagtagaagtacaatgtaagccatgttttgtttaacccagcatatccaaaatattaccccttttgcatgtgcttaatataaaaaattaagatgatttctattccaggttttcaatattcagtgagtaattttacacttagagcaagtgtcatttcagactagtcacattttgagtactcagtaaccacatatgggctagtgctaccttgctagatagcatagcctttgggtcccacaaagtgttcacattctctgtttacacacatcttttgaagtgtcatgagagagagccagataggatccagattttctttatatctgtttggtctgtcttacggattcctagttgattatttctttgttgcttcttatagggatgtatcagaagcttaataaactcatgattattatgttaactgcctgaagtattaattttagcaacaaattgaactataattttaatttttcaagtaaattttcttatgtgatactaagtatttcatatatacatgtattagaagtaaatgggagtttagtaatgaatagtatatagtagtttgtgattatatttttctgttttttttttttagattgcagcaaaaattggaggtgatgctgggacatcactgaattcaaatgactatggttacgggggacaaaaaagacctttagaagatggaggtaagttatactcatacgtattttaaattgtttttcagagtgtttagttgaagtggttctcagtattttttgttattctattgagacattacttttattataagattgttaaaactgtaaggtgtatattggcctaaagggggggaagagacctagagaaggtaagtaaaatttcagtcaaccagaaagttttgtcttactagaaaacagtttattttccctcttttaggattgtgccagattgaaagtttgcacagacgtcttgttaataatattaaaaaaacaaaaatataaattgcttagaagacacttcactggttttactcatacatgaatggattttaatatgctgtgttttcgtcatttttctatttgcaattacactttaaaggtcgaaattcgtattgtttgctactctagtgtgttgcaggttataccgttttttttaatgttctttgcttttagtacttttgtgtttcacgtttagctgtaaacctatagattaaaacagtgggttcacagtgctttgtagttttaaaaatcataatcattgaagcttctgaacttagaagtctgcatgtattttttgttttaggtttgatgtatgagttttgatacattttctttttacccttttttttaaaggaaggattctcactgaggctatagaatgtatttgtagcttttgaccaggagaactttgtttcctttatttaagtgtctttcatatttatagtgaggtttttaatataaaaaaaaaaaatgagctaatgatgcttcagatgttgtatgtaatgtattctttttattttatgtgtagatggctcttggacaagtccgagcagtacaacacactgggagggaatgccctctccttttaaaggcaggaatttttatttattacctgtgttcagtatgtaaacgtgaaataaaccagtggattcttaaatggacacaaatatttcttggattatgtgtctgaatttttgctgcacaacattctgatggttaatcatttaagtttgaaggggggaggagaatgtagtactttgagctataggttgtctgttccaaggtatgcattgtattcatctgtgtaatggatttagatgaaggtagtcatgtagttgccttgagtttttttgttttttgtgttttttttgctaaagttttatacagtgaaatgtttgtttataaataatagaacagtttaagttgagattgccttgtaatgttgtggggtttgtttttttttttaattttttggggcatagctttgtggtcactgtccgatatactcaacttttaatatgtcagatttttgtagtttgatacgcttttttccccccagtcttcagttcctgaggtggaagcatcattagcctttagcatgtgatattttgctagtaatggacctaaagtactttgtcttgtatcattctaatgtgcataacatacattaaggtcagtgatttgttaagaatcagataactattttaatgtctgtgcatcttttgaatgtgaagagaatgaagtatagtttcttttttagattactgagtttgtgttgaattttggcagttttggttcaaatgataaaccgtaccttcagatatttcaataaatgtttatatttattcttgtttgggaggggagaagctttgtacttaattggtaaaaaattaaaagacacttaattttgcagatcaaccagatgctaagaaagttgctcctcaaaatgactgtaagtattccttttaaactgggtcaaaagctatattaagattttgttcatattattgggtatctttgaagttagttggtttatgagtattttggatcagctagcctgaacttctttgtaaatatgtacctttttctcctattttacaatctgtcccattaattgtggccaggtatatgtaaggattggttgccgaattattttacatatttaacaactccaattcttgatctacgcttgtacaacttgaaaaaggaaattatattgttctgtgccattgctaatataatgtcttccctttcattggctctcttacccctgtcagtgccaatataaatattctgtataaaatttgactctcttcaaggtgttgtctgtgcattagggaaaggtttttgaatgtttgatgtgattttgttgttaaatacataagtaactttaaattttgaattttcgttttatttttatttaattttttgatagcttttggaacacagttaccaccgatgcatcagcagcaaaggtatagtcacaaacttttcaaaaagtactctgcaagttttggttgagctgcatgtaaaaacacaaccacattggtatatattgaatatgtgtctgtattttttggtgtacctagttcatatcactccccttgggaaggtaccataaagtgatgatttttcttttgagtgagaaaaatttgtgatttggagagagagagggaattagccacagtttagaagaacaggggtgaattagagtaactgttaagatgacattctctnnnnnnnnnnnnnnnnnnnnnacagttcatgccttcagactgttccattgatcatcccttcttcacttgatgtatcatctnnnnnnnnnnnnnnnnnnnnnnnnnnnnnnnnnnnnnnattttttgacatgagtctgagcatagaaccttagtttaagccattgggagacattagacttccatttttattaatagattatcttttatttgtaaacaaagtatctttcactgaaggaaaaatggtactttctgttactttttagcagatctgtaatgacagaagaatacaaagttccagatggaatggttggatttagtaagtaacttgatttttaaagttttgaaaacatgatcaaaacatactttagaatctttcaaccaaaaaaactttttttttctaactagtaattggcagaggaggtgaacagatctcacgcatacaacaggaatctggatgcaaaatacagatagctcctggtaatgttacattctcttggtattttcagagtgactagaaaagtagctttttttttttcttttaggtttctagtactaataatgctgcctttaatcttttgacctgaagttctatttttgttttaaattgtagacagtggtggccttccagaaaggtcctgtatgttaactggaacacctgaatctgtccagtaagtttgaaaatctttaaaatggacttaaagtaacaacgggagaactctttgaattttctctctgctctttgttactgctttattttacactactctttcgttgcctccttccctcccaagtcctctgactcctttgaagtttatgcctcatgcctttctcaattagggtttatcattaaacacagaaaatggttaaaacaacttcatatctactccagtctctacttacaaagcgaagtgtagcctggaggagaatgcgcagtaatgttgactggtgacacctaaaactcagacattaagctcaagtggactgttgtgttgtctgcatttccctagttccattcacttttccattcctctccaaggctctttaatactatatttccccatctccaaatcttcagcatctaaccccacctctctccttcttaagcttatttacggagaaaatggaaacgaatgataagaagcttttcttttcccctaccgctaaacctaccagccttcattttttttcnctgttcacatagtactcaggtaattgctttcctttgtgttcatgtgcctaaagccagccctttcttcctattgaaggttcagcctgcagttgtactttcttctgcgttattagttccccccctttactagatttttnnnnnnnnnnnnnnnnnnnnnnnnnnnnnnnnnnnnnnnnnnnntgcaggtgcatgccaccacacccagctaatttttgtatttttttagtaganncagggtttcaccatgttggccaggatggtgtcaatgtcttgacctcgttatccacccgcctcggcctccccaagtcctgggattataggcgtgagccactgcccccagcctgattttctttgtagcactttccaaataatacgttattcatttgacatgttatttttacttattttaatgaaacgaagccactccagcaggaaccctgtctctttgagtgttaccaccccattacctaaaatggcacttgcacatggttgatatttaagtatttgttgaatgaataattgtagcatatgagtaagtaaaatggtagtttaaaaatgtaaataaataaatcttttagttcttggaagaatcagtttaattctgagataactttagcattagagttcttggaaattgtggactattcttaaaaataaaaattgtatatctagaaaatttattgcctaatctctcaatctttgacccttgatggcattttctttcagttaaaagtaaaaacattgttaaagttagcattaaggcacctaatcctgaattggggtaggaggagtacttggttacattgttttgtatttctctatttgaataaacttgggtatgctgcaacttactatttaaatattaatttgttaacaggtcagcaaaacggttactggaccagattgttgaaaaaggaagaccagctcctggcttccatcatggcgatggaccgggaaatgcagttcaagaaatcatgattcctgctagcaaggcaggattagtcattggaaaggggggagaaactattaaacagcttcaggtattgttatgtttgtgaaatggctacttttggtctgttttgatgnnnnnnnnngtctgctcccttttgttaatatgtattattttctatgattataacaggaacgggcgggagttaaaatggttatgattcaagacgggccgcaaaacactggtgctgacaaacctcttaggattacaggagacccatataaagttcaagtaaacttaactttatactttataaagaaagagtttgggttgaatggggttgggcaaaatatgcatgaataattaaaatgttttgagacgtgctttctaaattagctaactttttctactttagcaagccaaggaaatggtgttagagttaattcgtgatcaaggtggtttcagagaagttcggaatgagtatgggtcaagaataggaggaaatgaagggatagatgtaagtaaaaatacccattcagaaatggttgtatgctaattcgtaaatatagtagtgttttctgttttgtgttaaatagctctaacattgttatccttttatttcacctttatactttagaatacagaattaagtattttatatcttgtcaccctatttgctataaatataaaattatatgtactattatgatttgaggcagattttcaggaaatggtnnnnnnnnnnnnnnncttttttttactttaaaccctgagaagctagttttcttaatactcagtcttttttacataaggtccccattccaagatttgctgttggcattgtaataggaagaaacggagagatgatcaaaaaaatacaaaatgatgctggtgttcgaattcagtttaagccaggtgagtgcatataataatcttgtaagtgttggcagcaatgagttttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacacccatttactgtttaattaattttgtttctttgttttgaagatgatgggacaacacctgagaggatagcacaaataacaggacctccagaccgatgtcaacatgctgcagaaattattacagaccttcttcgaagtgttcaggtttgatagaaagttaacattttcattttttgtttttatggaaaaatactttccttcatgaaatctgaagtttcctctatatcagagtctgcttgatgatgctttattaatggagaaagtttaaattgttttaaggtaaagatcttggagcaggaagaactactaccttaagtgctaccttatttactctttaatttaaaaaaatgttattacttatgattatttgggccggttcatctctacatttatggttcaggtatttgagagctgggaaaaatagacataattaattttatcatgaaaaaaagtatttctgtgctgattttttccttcttgtctattcctctctataggctggtaatcctggtggacctggacctggtggtcgaggaagaggtagaggtcaaggcaactggaacatgggaccacctggtggactacaggaatttaattttattgtgccaactgggaaaactggattaataataggaaaaggcaatgtattttaaactcttaatgtttaacacattattcatttttctggacactatttctgttgctgttgtaaacaagtggcaatgctttttctctggctgtgttttagtagaaaagcattcttatgttaatacgtaacaagtaaaacataaatgaaggatctaatgtatatttataaaaagagcaggattttaatcttactagcttctagagaaagtgaactaagataattattagcataagaaaggtcttttgacccaaaaagttgtttgagtgtttttgtttgtgcattttggtttttcccgactcatattttaaaaatttgaatgtttataagtgtattagtttatatttatctgcttttaaaagcagtttattcaaatattttattataatcacattaaggttaagtttaaacataccaagtaaatgtaaatgtattttaagagaagcatgaaatgcttcctaaaatttagatttaaagtgtagaatattaaatgaaaaatcttaatacaatactgtcaagtagaatactgactgaccaaaccatgtttttaatggcctatttaattgtgaccattttcttctaaatagcttctagtatacccttgaaacctttagagaaattactgtcttttattttaggaggtgaaaccataaaaagcataagccaacagtctggtgcaagaatagagcttcagagaaatcctccaccaaatgcagatcctaatatgaagttatttacaattcgtggcactccacaacagatagattatgctcggcaactcatagaagaaaagattggtgtgagtatactttaaacttttaatttatagtgtagacccttagattgtagttaaattaagacgtttattcgaatacannnnnnnnnnnnnnnnnnnnnnnnnnnnnnnnttatagatttcatgatacctataatagatacaatgtgaagattttccagcaatgaaaataacctaattaaatgtgcagttacaggttttgagaacaaccttacgtttgagtgtggatagataggagggtgcaggcatcatattagtgttattgtaggattgtggaacatacttgaaggacacagttaatgggaattcattatttattaagattttactatactgaaccccagcaaggcaaacaagataaatcagatgcatcttccgctctgcagtagaaattcgtaaatcctagcttttggactggctcacaaatcatctgggttttaaaatgtagactatttgggtgcttccaactcctacacataatatacagagacagacctgactcaaaatgtctgggatagggcccagcatctgattttacacagatgtttgggtttttttcttccagaaaagttttctgttagaacaaaaagtataaaaagctttgactgcttttttgtaaatgaggcagatggtgtcttactggagtttttaacacaaagtgtgcagggagcatcttaaattattaatcagaatttcctaggaaaaattagttttggtgtttgccatgctgtgaatggagtgcatgcaaaacaaggttgacgctgttttctgtgtacttgggaagacaaaaatagatgtactaaagatgcttaaggaacatcttataaaatgtacacaaacattgtaagttctgttgtatggatagtttaattgttcaaaaaatgaagatgtttgtggagtaatgataggaggttgatgccatagcaaaaaaaaatgaatagcacatcttggtttttgattttacagtattagccttacatgcatggatcaggatttctgttggattcatggtaaaacaagatagattgttttagagaagcaatttggtgtggtgattaagagcacggactcttttctctgaatcatttactgggtctgtgaccttggagaagttgctcaggcttttctgtgccttggtttcctcctataaaatgaggataattgtatctatttcatagagttgtagggattaaatgaacgttcacctgtgtgtcacttaaaagaatgcctggcacatagccctaaaaaatgttgctactttttcagtattatttttactatttggaaagaatagataatggataataaagtgagagatgacagcaattggagacttaaagagacaagttaataaaaataactttaaaggtcatgaagtttagtttccctattttgcagtgagaaatttacaacaaaatatagtgttaccattttgtccagcatgtcgcctggttgtgttaactactcagaaggagcattttaggacagttaagtgtaatgtctttgttggcttaacaaaacagaaatcagttaagcattattaatnnngatgtggcatgcatgcatgataaggatataaaatatcctgatttattgaaggacttaaaaagggaatgtttgtgctgtttagaattatgatacatgaaaggccaaaaaggatataaattattgatctgaatggggtttcagtgacagaaataggttgtgaggtggagtttggtttaatagtgaaccaactagctgttaacattatcaatgaagtgttcagaagacaattgatgatataggacccaagcagtttgggaatatattgtcaagatggttgtcttatgttaggcaagtagatgtagagagaagagctgaagataaagacctgatttctgtgattaagatgaaaaaagatagtaaaagaaagaattataaaaggaaagggatctactgctgtcgcagacaagctaaagaaaatttaattgctaattttaatttaatttaaattgcatttaattgctaccgctattgattttagtaaatttcacatgtctccttattatggcagataaaatagtttttgcaaaatgaatgaggaaaggaaggaaaacctaaaatttgttgtttgtgactataagagggtagaaatggaagattatttgtccatagagttggtttgctgatttttctatgtcttcctttttttttttgtagggcccagtaaatcctttagggccacctgtaccccatgggccccatggcgtcccaggcccccatggacctcctgggcctccagggcctggaactccaatgggaccatataaccctgcaccttataatcctggaccaccaggcccagctcctcagtaagtattgggtttagttctgggctttcccccaaagattctagtttttggactacatttttatactgaattttcttctcagtggtcctccagccccatacgctccccagggatggggaaatgcatatccacactggcagcagcaggctcctcctgatccaggtaaaaagatgcttatcatttgtgtgttagctgtattgtttttcactcgtgttacattattaaattttctagtgttgattctacatttgtatgcctcaccttcactcactctactctttcaacagcgttaggcactgcctctaccccagtgtatatagaactgacatgaatatgatctctgcttttatgaaattcctttcagcttgcatttggctttcttatgagtcgatatagcagaatgataaaaactaaaagctgcannnnnnnnnnnnnnnnnnnnnnnnnnnnnnnnnnnnnnnnnnnnnnnnnnnnnnnnnnnaatatgtagtgtgttaattgtgacattatctatgaaaaaatagtttttacnnnnnnnnnnnnnnnnnnnnnnnnnnngggcactgaggctcacgtctgtaatcccacctgttcagaagtcgaggctgaggcttgagaccagcctaggcaacatggcaagacccatctctaaaaaaatttgttttaaattagccagccagggtggtatgtgtctgtatttctagctgctcagaaggctggagggggggcggggattgcttgaggctagaagttctagattgtattgagctgtgggcgcaccactgcactcccctgggcaacagaatgggaccccatctcttaaaaaatatatatatatgtatatataaaacagtcttacagagtgttaagtatttgggcattaaactcccaaattgtcaaataagataccggttctagtactcctcataatgacaacttcatgtgagtaaaaatcaggcctgtatttaataactgcatgctaaaacccaaatacatttaattattttctatattcagatctgtattttcgatatgtatctattatatcctgtactattctggcgtctttccataccctgtagtttgctttccatcttggtcaaagagtcattctttgaaaccagttaacatttatttatgatctttttttttcacctgctacctaccttttaggcctctttcaccagagtagtttcaaggaaaatatactcaattatggattactttctacacataatnnnnnnnnnnnnnnnnnnnnnnnnnnnnnnnnnnnnnnnnnnnnnnnnnnnnnnnnnnnnnnnnnnnnntgctgtttggaatatgatgacacagcatgatagttaccaaataatcacaacttccattaattcaaggaatgacttgagttgtgctaggatataggtatgcaaagttggggttgtattctatagcaaaagaatgcactcccagcataccaacactgatagaagggctcacaaggtacagaatataatactgtaaacacgaatttttggagaatgaaagggctttgcttttcctcttgttggctaattgggatggtataattaagtaatgagattgaagagtttgagtaggttaagagacagattcatactgggtaacttggatctgccaggattgtatttttgagcactactgggtggttagcatgattgagaaaaaatgatgggnnnnnnnnnnnnaagtggtctttgtatcacatgttgagtttcccacttggagtagtagtgaagtcactactataaaagctggtcagtgaatgtggttgcagcatggcctttgggcaagaagtaacccatttaaanccagctggttggccccactcagatttatcaaagggttactgggtctctggggtggatattgcttatattagacttagaatgacgtaacgttttaatgtatgaactaaaatatttctttaaaaaaagagtggtctgttacggatttatgtagtggtcaagaatttagacttcagagtcaaatagacctagatcagtcctagtcctacagtttactaatggtgagatgtcaggcaagtttttgaactcctctaagcctgttttcttatctataaattgatcaatgaatgaatcttgggttgagtgaatatttagtaaattcttagtacatactagttatttgtgagactggtacttcagtatggtttacacgtttgggtgtagaaataacacttcctaaagtctgttttatctcaaattctctgtccaggcatagtgtaaagtgaaatacctagatttcttgattaatatacagataatagccgggcgccatggctaacacctgtaatcctagtactttgggagactgaggcgggcggttcacttgaggtcaggagttggagaccagcctagccaacatggcgaaaacctgtctctactaaaaatacaaaaattagctgggtgtggtggcgggtgcctgtaatgccagctacttgggaggcnnnnnnnggagaattgcttgagaatcgctccactgcattccagcctgggcaacagagtgagacacttcatctcaaaaaataataataaataccgataatgacactattgagatatgtaaacatccaggatacaaaagcagtacattgggcaattgagaaaagcttggagggtgtcctaaaaacaccttacaattatattaccttgtagttttctttttctacaaattctcactcctttttctcaaacttgactaacctttgttaagcagctgagaattgctactgttcagaatgaaagcataatagaaaatttaaaagttttaattgtatgatattcctagtataaaggacagaatcaagtttttttgtggtttctagaagattgagaggtctcaaactgtttgcacttcagttgatgtgggagatgagtgagggtcagtcaagtgtagaggaaacatagctaaaagctgagacatgggcatagtgatttctgaaaagtacaagcactgtgttgtggctggagcttgggtgttaaagagatacaaatgaagggagaggtgaggctgaaatggaaaggataagccaggggattcagactgttaaagatgttgaatgataggctgtacttaagctttgaccatcctgaagattctagagaatgaagaatttcaagtaggatgacatcaagtttataacattcaatgtgaaataggatgaaatgtggctaatcttttttttaagatttttatattttctcttcattgaaaataaggacaaagttcattgttctaaaataattgtttctttcttatgcaggttagcccctagaaatgtttttcttaagtcatcttccagatagagctgtttgtgcttgaggcgaaaccaatttagaaaaaaacaagggcacaggatggtttgagacagagcattggatttggagccaaaagacctttattcatatcccagttatgcaactcagtagtttttaacctgggaaagtcacttggtctctgagccttgttttctttaataaaagctgatggtaacaacaaaaataatagaaaatataaaanntttcatgtacttttttttaatgttctagttaattttgggagctgtatatttgccagagagctgggggggnnnnnnnnnnnnnnnnnnnnnnnnnnnnngtgagaattacagacttcaacatgcaaaccttgaactttcatttattctagctaaggcaggaacggatccaaattcagcagcttgggctgcttattatgctcactattatcaacagcaagcacagccaccaccagcagcccctgcaggtgcaccaactacaactcaaactaatggacaaggtaactaaagaacttatatgtgaaagtcaaaacttgtgcttggaattatatatgaagtacatcactgtataatacctaaaatttctaacattatttaattataatttaagcagacttttccttttttaaattgttacctggaaatagtttcaaactttcataaaagttataagaataccaggagccacccatataaccagctgttaatgttttatcccttgtttactctaataccgtctatatcatacaagtgtatgggtgtgtatttattttaaatatccattataattagttctgaaggaaactaattatatccagtataattagttctgaaagaaatcatttaagactaaactatagacacaatgtcctgttgggtacttcttcaaaaatatataaccatcatacagccctccaaatcagtaagtcaacattaatatattaagctctttatatcattatggatttctgatttttgcagtgggttatattttttgaatatttttataattttgataatcagactatcctagatttggccagtcagggagtatattcagggtggtgcctatatccttttgaaatatctgtcgttattttaagcacttccatactgtctggcacagtgagattgttattttgtgttttatgggctccaacccttaagtcagctatttcttcaaggagctctgattccttttagtagagtatggtagttagaaacgaggcttgactatgcttgttgctactgaggtgtaattgcttctaacttctttcagcagacagaactaggaaatatatttacatacatgcatacctacatacacatgccaaaaaacacataaacattgatatgtgtatatantttttaaaactatgttcatattacgacattcatttcagcattttggggttttcaagccttttccttttctgtacttgtttacaaatggtgagaaacctggtatcctcagtgtatctccttattttacatacatttacttatatgttcgacataaccagtcttccaaacaggttggttttcttttctgtccaccacctctgtacccccagtaccttctatctttggcaccagtagggatgccaccaccacatagtaccttcctcctacccctcactgtcacattgcaggcccctgccagctcctgcacccaaggaaacagctaaaattgccttttaaaaactttaattctgtttttctgttttgttttgttttgttttgcttgatcaaggagatcagcagaatccagccccagctggacaggttgattataccaaggcttgggaagagtactacaagaaaatgggtatgttttatacatttcttgaaaatacatacttaattaaattgaaacacaaggtattctcttcagaaagagaataattgaataaaatcactggactcgtaaacataccaagacagttgcaaattatagtttttaaatttgtggttatatagcaaggaaatatttttctttctaattgcatttgtcaaccagttattaattgaaactagaaatttcctcactggcacatacagtattaacattactatacttttgacaatgacagttatatatattagtctgagtgacataaggttaaattttaatgtgtcaggtgaaaatggattgtgtgtgataccattatttttgctgcaagataagcaggtaagaagtaatctgtagtgagggaaagtaactaagtgatggaaccagaatctggcttccaagagggtctgagtcccaagcttgtctcccaaatttgtctctttagggaccatttggaacctggtactatacttctggacaaatcactatgtttcaactgcttttgctctttaaaaattattacataccacagctagatgtcacaaacgaaaactaaattggtaagcttggttatccttcactagcagaaaaagaatctataggtggtagttttgtcataagagatcggtctacttgggattctcagtgtaagtttcaatgttttctttgccaaagaatgtttctcattctccgcagaaagaaaaatttccagaagggtgatgattttaatcttctaggtgtaaaattatatatacttgatgataaagatgttctgcacaactggtttctttttaaagaaagaaaaaattgtttttcctcatagagtggcttcctaggagagtcatattccatctatttctgagactataacagattaatatatgtttttgtgacgtagcttaggcagatctacagtagctgaaattccgcaaaaagaaacttttaacttaaaaacagcatactctgattaaggttggttacataatatattttctgaacagggatcttttttagaatgaatagggatgctattaatcatgtcctgncgggcgcggtggctcacgcctgtaatcccagcactttgggaggccgaggcnnnnnnnnnnnnnnnnnnnnnnnnnnnnnnnnnnnnnnnnnnnnnnnnnnnnnnncgtctctactaaaaatacaaaaaattaaccgggcgcgatggcgggcgcctgtagtcccagctactcaggagcctgaggcagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaaaaatcttcaacttctattttctcaatgggtaatgttccctagatatggtccctagttattannnnnttaagtagataaaaataaaggcttgttaatggatttagctaattactgaggaatgagtttgacttctgnnnnnnnttttaccttattgattatttgtaatatggccgttaatacatttactgtttagtcttttatgttttacttttttatgttttactcnntgagtgggtgggttgaattctgatttttattgttaagggaagaaattctaattttcattgttaagggaagaaattgtctttattgtctttaattttttttttcaccatttccctgccagttagagatactgtgctatactgtcttaaatcctctgtaggaaaacatggcatagaaataattaaataataatactgcatgactaaagaacctccttaccccaccttttctatcatttttctgtagtaccacaataatcacctgttaattttatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcacctgttaattttatgagcaaaatagattagttgaaaacagatgtaggttaagttttccaaaattggctttatattttaaagacaattctaaattccccaggataacaccaaactaagttatttttttttttntttttttttnnnnnnnnnnnnnnnnnnnnnnnnnnnnnnnnnnnnnnnnnnnnnnnnnnnnnnnnnnnnnnnnnnnnnnnnnnnnggttcaagcaattcttctgcctcagcctcccaagtagagaaaatcttgatgagatttcagagagttataaggaatggcagcaatctaaagatgggtatttttaaagctagaatagaaggattttttataagagactcttaagagaacaagcagggtttcaaagtagaaaggagtaattcttttttggagaagtgaagacatttgagaaaagtttgacttaaggaaggcccttacaataaggactgatgtagtagactctgaacccttaagaatctggtaccttaagcctaaaggagggtagagtgagacttgtaggaaaggtacaagtatataggaagacaactgagtgtgaaaatctattcaagcaaggctgactcttaggctataataaaatcttacaggctaactgtagagcaagtcttttatcttttggatgaaagaaatgtaaggctaaactttatagattataattatnaaactataataaatattagataatgttctactgaagtgtaaactcaactgtcttgaatcaaacagtaactaagccatgatcacaccactaccactgtgttccagcctaggtgacagagcaagaccctgtctcaaaacaaaatccaataactaaagagaataataaggggagcaaggtaaggcagtacatgtctgaaagggcaaggaaagagttctttaaagtctaaagtgtttactattagctttccacagggatataggagtttgcagacgttgtagttttttaagggaacattgatggaattttcttgatgaatatgcctgtttctaccatcttacatctaaaatgagtaactctcacagtgggtttaaaacctgaagaatattgattgctaccttgatcaggtttggtttcaagtgtgcagcttgtaagagaaaacgtgtggttttattgctgttgttacacgagagtatttggcagttttttaaatattgaaacctgtttcagatttgcttgaattgtgtcttgtaagaggcaaatgttcctgtttgattaacagttctttatagaagacaggaaaatatgagaatttttaatagtctatgagagttctctgttacccaagaaaagagggtttgtttcatgcatttttaaagaatcataaatcttaaattctttcactcagttgctttgattctgtcacctattttacaatggtgatggacaggtttctgaaactaggaaattggccttgttggcaacatcatacaaacataaaaacagcttcctatacgtccatgctccagtacttttgtctgtggtaattatttcacaaagccaagtgagttacaaacgaaataaaatagtgcttaagtaaagcaactgaataggaaaacacatctctcttctcaattttttatttagaaaattatataccttcagaaattggggaaattaggtaccactcagcttcattcatcatttttttaaagtttgctgtatgtgctttatatgtacgtttttctgtatgaaccatttcaatgtaagttgcagacaagacagttggctctaaagaaactcaatgtctggacacagtgattcacgcctgtaatcccaggacttttgggagaccaaggcaggtggatcactttagcccaggagttaaagaccagcctggacaacgctgcaaaaccccatctctgcaaaaaaagagaaaaatnncagctaggcatggtgggcttgtagtcccagctactctagaggcagaagtgggaggatcatctgagccgtgggaggtcgaggctgcagcgagccatgaatgcaccgctgcactccagcctgggtggcatagtgagaccctgtctgaaaataaataccttcaatagtatcacaaaaatacagactatatttaaatttccatagttgtcaggaatatgtctttcgtcgtctgtgtgtttgttaaattcagagtgcattcaaagattcgtgcattgcatttcattgttatatttctttagtctcttaattcagcaaagtcatctcatttggagaaagacctgatcgtgaaagctgagttatgtgtttcattgatcttgttctttattccttgtcacgttacttttaataccattgtgttgtaattggaataaattattgatacttttaacatcttactactaatctctgtgtgtgtgtacatgtacatgtacatacactttataggttactaatcaggaaaaagtcttggctaggtcttaaaataataaaatctttaaatcattgcaattaagtgggttttttgtttttaattctggaatccaatcaagattatgcctaaatctttaccttccttagctaaagcagtgtggatttggggttgattttgtttttttactaataatgacgctctagaactaaacattatgattaattataaggcaaaaagaaaataaagagcgttttttttttattttagagtagtttcctgacactactaatgaatattttaaataaaacaggagtaattctgaccctctgtgcttttgtcttatgattgtaatttaatattaaatttagggggtttttttaggtcaacaagggcagacacaagattattcaaaggcttgggaggaatattacaagaagcaaggtattgtttttactggaaatgaggtgttggacttttaattgtggttacagcaacaaaattcttttttttcaaaggtcaagcagttcctgctccgactggggctcctccaggtggtcagccagattatagtgcagcctgggctgagtattatagacaacaagcagcctattatgcccagacaagtccccagggaatgccacagcatcctccagcacctcaggtataatgtaattgctaatttgttgatttctactccagtctgttttctgcatgtttactgtttgtctgtttgggagtgtttgccttttaaatttttatctggcaaagtataacttatttaaatgaagtactacggtgtattgtttgggtttttttgtttgttttttataatgttttccggcatctgagtgctgaatatttctgcaatgcctttgattttaaaaataaattttcttcccccagggatttgcaaatcatgcaagaagctaccaccatttatattaaccagtttttctttcttaaaggattcactcctgaattagctccatttcaaggattttctttaactttttgtgtatttcttatgtatctcttctgcacagggccaataataagaagtggacaatacagtatttgcttcattgtgtgggggaaaaaaacctttgttaaatatatggatgcagacgacttgatgaagatcttaattttgtttttggtttaaaatagtgtttttttttgtttttttttgttttttttttttgcaaatgtacaaaatatctatcactactgataggaggttaatatttctgtgtagaaatgaaaattggtttgtttttagtatttagtgtagatgtacacattccagcaaatgtatttgcaattatgtggttgatgctttgtgatataaatgtactttttcaatgtatactttcacttttaaaatgcctgttttgtgctttacaataaatgatatgaaacctcctgtgtcggtaagttggatatgtgggtatttaaaggattcataatttcttagcaatgataaattaagatacatatacacaaatatataagctttccccatgaaatattgagtttttaaacactggcatgtttttccccccttgcagtatagtggtagattggaggatcttttccatttattgtatttggctctttcagcacaagtaatcctggtatcttcatttttttccttctgtttgattaaaaactgcatgtgtgtacaatgatcttctggcatacttccattgcattaacagtgaaatttcctttttatacatgaccactgtttcagacctgtactgctgctataacagttaacctttctgttcttaatttgataatacttgatttccaagactgtttcggcataactaattttaaacagttttcagatagtgaatatgagtagtctaataagaacagtttttttccatgtaaagcaactctttcaatgtatataatagtgtgtttctttctaaatttaggatagaaaagtgaatagtgtgcaaaaagtatagctacattgcatctgccattgaaacataaatggggtatggaaacgttcaagctttttttttttctttaagcagtatagataagctttgttttgtaaatgcacaagtccaatcattgaatcaacttaatttttttatgtacttgaagtcattttattactctttaacactcatgctgaagttctgatattttgttgaaatccattgttttactctttgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagaatgacaaattgatgaactgcaacagtatctttttgtcaatgttccacataatgcaaatgccatactttgtgtgaatattatgttggaatacagtgctgatatcttggaaaaccataactgcttcttaatttaacatagaataatacatagttctgtattttttttaaagtgagcttaatgggtaagtattttttatatgctttagctatagctaaagaaaactgatacttaacaaagttgaatagtattattcactggtgctcctgaaatattgtttttcagtgtaaaatatgcatatattctatatttaatatgaaagtcttgaaatgtatcagaagggatttcagtttgcaaataatgagcaatgtagcaattttaacacatttcataaatatatattttgtcatttgtggggagcaccatttgttgttttgaatatactttaaaggaagaggtacaaggacataaatgttgagattacctacaggatggaaatagcagtacagttcattatagatattttgaaatgtttttgattgttttatataacctagagtgacttcccttacccttatttagatctggatatatagttctagtttgaagtttaatagttaaggagttagctatttgttatctttaagagtagggtattgacgtgagcaattgcagtattttgcatgatactgttttatagatgaccttttaggaaagtggtgcatttattaattgaactgaagaagtagttcagttgaattcagtatcataactcacaaattggaggctgttgattttgattcatttaaggtttaaaatctttattaattgcaaacagtgcaattatttatacttcacagtgccttcccagaccttccaccttaggttctgctgcaaaaagcaccaggtaagcacaacctaaggacatatataaataaatatttcagtacattaatgttgtccctgtgaggtttttgtggttgtgtattcaaaggcaatctgctnnnnnnnnnnnnnnnnnnnnnnnnnnnnnnnnnnnnnnnnnnnnnnnnnnnnnnnnnnnnnnnnnnnnnnnnnnnccatcttagtggaaagtctgtaagttgttaaagcaactgtttacatttctgggtaatgtttttatttacgttttttttttttatttaagacaagaaaatgatgagtagattgctgcagtaattgatctacatccaaatctttttgtattttttccccaaatatagaagtgttaagattaagaaaggacaattacacagttttcaagatttaggaaatcacttgttcagaaacttcaacagccttcacaatctgttttatatgatggacagaaaatttctttgccctccaaaactataatttctttatttttttcttaaactataataattcagtaaggatattatgggttacaattttattatgttttttctgagacaaaagttatatgctagaaaaggaaaaagttaataaggcagtatgttttgataaaaggcatgtgcatcagtgaaatgttaactgtacagcaaataacctttcataatctgtagcaatcagtatttttctgatttaatattttaataactgacgctgcatttatataatttttttgccagtttaaaatgtttgtgtgtttttatagatgattttaactggtacatattttgagttaagatgaatgtatgaaagcagcatcttaccagttttgtttattcaatttctaaaatgtgctgatccttttaaaactcctgcttatctctgcaacaaagaaaaatattcaaaaatactgccttcattttcacacacagtgctgaagatgctgcaagcaccaaatcatagctcaataaaatcaggtcctgagatagttacccataaagaggaatcctttgagtgtatgccattggtgagccgatgagcatggaccatagaagggctcaatgtagaaggtaaaattggcaaatcataattgagaaatatgaaatgtattcccatacataatatggtatagggtgtaatgtacctgcttttgatcacttttcattttaaagtgctattcacttgatcttaaatgttccatgaactgttaaatttcttaagttacatagttattacaccacatttatgtgtatgttatgttttaatagtcaatgataggtatgtaaatataaagggactcattgaaacttgagagcctgtcgagttttggttagttgtagattgcatttttattaaaaaaatatagatagatgaatgataatagatattggggcactgtttctgtctcatgagaattcttttattcattaccataagccttcactgataatataagcattattttaaatgacgctggtcttaaatctgaaataaatggaaagcagaaaaggtgagccagttgatttgaatgcattggatattagtgttagaaacaatgtatagtttagattgaaattgaactgactttatttagcacttaaacaaaaatttgacaatgtttttggtttttttttaagacagcttagtgtggtgatacttagaattctatggtttgatgtttcttttagaaatgagaagtatagttttattttttaatatcaaaaatggttttaatactaaaactagtaatttaatactagttgtttataaacattgtaaaatatatcttttaaacaaattatcttagtagttaattcataagggtggttttgggtaggaatagcagagtaccttcagagggaaaggggagtaattcagaagtgatagcattttatttgtttgaatactctgccagtaaaatcagctctacttagaaagttatctgttgtgtagaataatgatgtagagtttactaatcagtgaggatgtcttgtttttattttctgcaaactctgcctcactttaaaatgcattttaacaatacctaattaaaaataattttggttctgaaaataaccttattttttgttgagttagtgacttcatttttcttgtcacaatataagcttttgagggatttttttaaattggtgcttttaataagcaaatcccagggttttattttcttcagtgatacccctcttaaatgtatttgcacatatatatatattttttcttatgcatgctcgatgcattttcgtcctgagaaaaatgttctctacagaaactacccgtgtgtaaaaagaagattggcttaaaatggctactgtgatgggaacagtgtcttagggagatgcagcttggacttgaggtaaattgaatactttacaaactgtggtttagagtttgctttaatgacattgtatgtaaaaggtcacatgatagctgtaattttgtattcattatggtttcctcaataaataaatgtacattgatgaatattataag

SEQ ID NO: 252 - DNA, 1968 bp, Macaca fascicularis
  source 1..1968: mol_type = other DNA
atggcagactattcaacagtgcctccaccgtcttctggctcagctggtggcggcggtggcggcggtggtggtggaggagttaacgacgctttcaaagatgcactacagagagcccggcagattgcagcaaaaattggaggtgatgctgggacatcactgaattcaaatgactatggttacgggggacaaaaaagacctttagaagatggagatcaaccagatgctaagaaagttgctcctcaaaatgactcttttggaacacagttaccaccgatgcatcagcagcaaagcagatctgtaatgacagaagaatacaaagttccagatggaatggttggatttataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattcctgctagcaaggcaggattagtcattggaaaggggggagaaactattaaacagcttcaggaacgggcgggagttaaaatggttatgattcaagacgggccgcaaaacactggtgctgacaaacctcttaggattacaggagacccatataaagttcaacaagccaaggaaatggtgttagagttaattcgtgatcaaggtggtttcagagaagttcggaatgagtatgggtcaagaataggaggaaatgaagggatagatgtccccattccaagatttgctgttggcattgtaataggaagaaacggagagatgatcaaaaaaatacaaaatgatgctggtgttcgaattcagtttaagccagatgatgggacaacacctgagaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccaacagtctggtgcaagaatagagcttcagagaaatcctccaccaaatgcagatcctaatatgaagttatttacaattcgtggcactccacaacagatagattatgctcggcaactcatagaagaaaagattggtggcccagtaaatcctttagggccacctgtaccccatgggccccatggcgtcccaggcccccatggacctcctgggcctccagggcctggaactccaatgggaccatataaccctgcaccttataatcctggaccaccaggcccagctcctcatggtcctccagccccatacgctccccagggatggggaaatgcatatccacactggcagcagcaggctcctcctgatccagctaaggcaggaacggatccaaattcagcagcttgggctgcttattatgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaagcagttcctgctccgactggggctcctccaggtggtcagccagattatagtgcagcctgggctgagtattatagacaacaagcagcctattatgcccagacaagtccccagggaatgccacagcatcctccagcacctcagggatttgcaaatcatgcaagaagctaccaccatttatattaa

SEQ ID NO: 253 - DNA, 6825 bp, Macaca fascicularis
  source 1..6825: mol_type = other DNA
ccttagccccacactagcgcgttgcccggaaacatcgcgagatttcgttccttcagtctccacccctttacggcacgatggtcgcgcaagaatgtgatacagcttcgacggccgccattttctttctttcttagctgttagctgagaggaagtctctgaacgggcggcagcggctagcgtagtgtaaccatggcagactattcaacagtgcctccaccgtcttctggctcagctggtggcggcggtggcggcggtggtggtggaggagttaacgacgctttcaaagatgcactacagagagcccggcagattgcagcaaaaattggaggtgatgctgggacatcactgaattcaaatgactatggttacgggggacaaaaaagacctttagaagatggagatcaaccagatgctaagaaagttgctcctcaaaatgactcttttggaacacagttaccaccgatgcatcagcagcaaagatctgtaatgacagaagaatacaaagttccagatggaatggttggatttataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattcctgctagcaaggcaggattagtcattggaaaggggggagaaactattaaacagcttcaggaacgggcgggagttaaaatggttatgattcaagacgggccgcaaaacactggtgctgacaaacctcttaggattacaggagacccatataaagttcaacaagccaaggaaatggtgttagagttaattcgtgatcaaggtggtttcagagaagttcggaatgagtatgggtcaagaataggaggaaatgaagggatagatgtccccattccaagatttgctgttggcattgtaataggaagaaacggagagatgatcaaaaaaatacaaaatgatgctggtgttcgaattcagtttaagccagatgatgggacaacacctgagaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccaacagtctggtgcaagaatagagcttcagagaaatcctccaccaaatgcagatcctaatatgaagttatttacaattcgtggcactccacaacagatagattatgctcggcaactcatagaagaaaagattggtggcccagtaaatcctttagggccacctgtaccccatgggccccatggcgtcccaggcccccatggacctcctgggcctccagggcctggaactccaatgggaccatataaccctgcaccttataatcctggaccaccaggcccagctcctcatggtcctccagccccatacgctccccagggatggggaaatgcatatccacactggcagcagcaggctcctcctgatccagctaaggcaggaacggatccaaattcagcagcttgggctgcttattatgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaagcagttcctgctccgactggggctcctccaggtggtcagccagattatagtgcagcctgggctgagtattatagacaacaagcagcctattatgcccagacaagtccccagggaatgccacagcatcctccagcacctcagggccaataataagaagtggacaatacagtatttgcttcattgtgtgggggaaaaaaacctttgttaaatatatggatgcagacgacttgatgaagatcttaattttgtttttggtttaaaatagtgttttttttttgttttttttttttgtttttttttttttgcaaatgtacaaaatatctatcactactgataggaggttaatatttctgtgtagaaatgaaaattggtttgtttttagtatttagtgtagatgtacacattccagcaaatgtatttgcaattatgtggttgatgctttgtgatataaatgtactttttcaatgtatactttcacttttaaaatgcctgttttgtgctttacaataaatgatatgaaacctcctgtgtcggtaagttggatatgtgggtatttaaaggattcataatttcttagcaatgataaattaagatacatatacacaaatatataagctttccccatgaaatattgagtttttaaacactggcatgtttttccccccttgcagtatagtggtagattggaggatcttttccatttattgtatttggctctttcagcacaagtaatcctggtatcttcattttttttccttctgtttgattaaaaactgcatgtgtgtacaatgatcttctggcatacttccattgcattaacagtgaaatttcctttttatacatgaccactgtttcagacctgtactgctgctataacagttaacctttctgttcttaatttgataatacttgatttccaagactgtttcggcataactaattttaaacagttttcagatagtgaatatgagtagtctaataagaacagttttttttccatgtaaagcaactctttcaatgtatataatagtgtgtttctttctaaatttaggatagaaaagtgaatagtgtgcaaaaagtatagctacattgcatctgccattgaaacataaatggggtatggaaacgttcaagctttttttttttctttaagcagtatagataagctttgttttgtaaatgcacaagtccaatcattgaatcaacttaatttttttatgtacttgaagtcattttattactctttaacactcatgctgaagttctgatattttgttgaaatccattgttttactctttgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagaatgacaaattgatgaactgcaacagtatctttttgtcaatgttccacataatgcaaatgccatactttgtgtgaatattatgttggaatacagtgctgatatcttggaaaaccataactgcttcttaatttaacatagaataatacatagttctgtattttttttaaagtgagcttaatgggtaagtattttttatatgctttagctatagctaaagaaaactgatacttaacaaagttgaatagtattattcactggtgctcctgaaatattgtttttcagtgtaaaatatgcatatattctatatttaatatgaaagtcttgaaatgtatcagaaggggatttcagtttgcaaataatgagcaatgtagcaattttaacacatttcataaatatatattttgtcatttgtggggagcaccatttgttgttttgaatatactttaaaggaagaggtacaaggacataaatgttgagattacctacaggatggaaatagcagtacagttcattatagatattttgaaatgtttttgattgttttatataacctagagtgacttcccttacccttatttagatctggatatatagttctagtttgaagtttaatagttaaggagttagctatttgttatctttaagagtagggtattgacgtgagcaattgcagtattttgcatgatactgttttatagatgaccttttaggaaagtggtgcatttattaattgaactgaagaagtagttcagttgaattcagtatcataactcacaaattggaggctgttgattttgattcatttaaggtttaaaatctttattaattgcaaacagtgcaattatttatacttcacagtgccttcccagaccttccaccttaggttctgctgcaaaaagcaccaggtaagcacaacctaaggacatatataaataaatatttcagtacattaatgttgtccctgtgaggtttttgtggttgtgtattcaaaggcaatctgctactgcttccccaaaatgtattttgttattttatgctaccatcttagtggaaagtctgtaagttgttaaagcaactgtttacatttctgggtaatgttttttattttacttttttttttttttattaagacaagaaaatgatgagtagattgctgcagtaattgatctacatccaaatctttttgtattttttccccaaatatagaagtgttaagattaagaaaggacaattacacagttttcaagatttaggaaatcacttgttcagaaacttcaacagccttcacaatctgttttatatgatggacagaaaatttctttgccctccaaaactataatttctttatttttttcttaaactataataattcagtaaggatattatgggttacaattttattatgtttttttctgagacaaaagttatatgctgaaaaaggaaaaagttataaggcagtatgttttgataaaaggcatgtgcatcagtgaaatgttaactgtacagcaaataacctttcataatctgtagcaatcagtatttttctgatttaataatattttaataactgacgctgcatttatataatttttttgccagtttaaaatgtttgtgtgtttttatagatgattttaactggtacatattttgagttaagatgaatgtatgaaagcagcatcttaccagttttgtttattcaatttctaaaatgtgctgatccttttaaaactcctgcttatctctgcaacaaagaaaaatattcaaaaatactgccttcattttcacacacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgaaatgtattcccatacataatatggtatagggtgtaatgtacctgcttttgatcacttttcattttaaagtgctattcacttgatcttaaatgttccatgaactgttaaatttcttaagttacatagttattacaccacatttatgtgtatgttatgttttaatagtcaatgataggtatgtaaatataaagggactcattgaaacttgagagcctgtcgagttttggttagttgtagattgcatttttattaaaaaaatatagatagatgaatgataatagatattggggcactgtttctgtctcatgagaattcttttattcattaccataagccttcactgataatataagcattattttaaatgacgctggtcttaaatctgaaataaatggaaagcagaaaaggtgagccagttgatttgaatgcattggatattagtgttagaaacaatgtatagtttagattgaaattgaactgactttatttagcacttaaacaaaaatttgacaatgtttttggttttttttttaagacagcttagtgtggtgatacttagaattctatggtttgatgtttcttttagaaatgagaagtatagttttattttttaatatcaaaaatggttttaatactaaaactagtaatttaatactagttgtttataaacattgtaaaatatatcttttaaacaaattatcttagtagttaattcataagggtggttttgggtaggaatagcagagtaccttcagagggaaaggggagtaattcagaagtgatagcattttatttgtttgaatactctgccagtaaaatcagctctacttagaaagttatctgttgtgtagaataatgatgtagagtttactaatcagtgaggatgtcttgtttttattttctgcaaactctgcctcactttaaaatgcattttaacaatacctaattaaaaataattttggttctgaaaataaccttattttttgttgagttagtgacttcatttttcttgtcacaatataagcttttgagggattttttaaaattggtgcttttaataagcaaatcccagggttttattttcttcagtgatacccctcttaaatgtatttgcacatatatatatattttttcttatgcatgctcgatgcattttcgtcctgagaaaaatgttctctacagaaactacccgtgtgtaaaaagaagattggcttaaaatggctactgtgatgggaacagtgtcttagggagatgcagcttggacttgaggtaaattgaatactttacaaactgtggtttagagtttgctttaatgacattgtatgtaaaaggtcacatgatagctgtaattttgtattcattatggtttcctcaataaataaatgtacattgatgaatatta

SEQ ID NO: 254 - DNA, 1959 bp, Macaca fascicularis
  source 1..1959: mol_type = other DNA
atggcagactattcaacagtgcctccaccgtcttctggctcagctggtggcggcggtggcggcggtggtggtggaggagttaacgacgctttcaaagatgcactacagagagcccggcagattgcagcaaaaattggaggtgatgctgggacatcactgaattcaaatgactatggttacgggggacaaaaaagacctttagaagatggagatggctcttggacaagtccgagcagtacaacacactgggagggaatgccctctccttttaaagatcaaccagatgctaagaaagttgctcctcaaaatgactgtaacagatctgtaatgacagaagaatacaaagttccagatggaatggttggatttataattggcagaggaggtgaacagatctcacgcatacaacaggaatctggatgcaaaatacagatagctcctgacagtggtggccttccagaaaggtcctgtatgttaactggaacacctgaatctgtccagtcagcaaaacggttactggaccagattgttgaaaaaggaagaccagctcctggcttccatcatggcgatggaccgggaaatgcagttcaagaaatcatgattcctgctagcaaggcaggattagtcattggaaaggggggagaaactattaaacagcttcaggaacgggcgggagttaaaatggttatgattcaagacgggccgcaaaacactggtgctgacaaacctcttaggattacaggagacccatataaagttcaacaagccaaggaaatggtgttagagttaattcgtgatcaaggtggtttcagagaagttcggaatgagtatgggtcaagaataggaggaaatgaagggatagatgtccccattccaagatttgctgttggcattgtaataggaagaaacggagagatgatcaaaaaaatacaaaatgatgctggtgttcgaattcagtttaagccagatgatgggacaacacctgagaggatagcacaaataacaggacctccagaccgatgtcaacatgctgcagaaattattacagaccttcttcgaagtgttcaggctggtaatcctggtggacctggacctggtggtcgaggaagaggtagaggtcaaggcaactggaacatgggaccacctggtggactacaggaatttaattttattgtgccaactgggaaaactggattaataataggaaaaggaggtgaaaccataaaaagcataagccaacagtctggtgcaagaatagagcttcagagaaatcctccaccaaatgcagatcctaatatgaagttatttacaattcgtggcactccacaacagatagattatgctcggcaactcatagaagaaaagattggtggcccagtaaatcctttagggccacctgtaccccatgggccccatggcgtcccaggcccccatggacctcctgggcctccagggcctggaactccaatgggaccatataaccctgcaccttataatcctggaccaccaggcccagctcctcatggtcctccagccccatacgctccccagggatggggaaatgcatatccacactggcagcagcaggctcctcctgatccagctaaggcaggaacggatccaaattcagcagcttgggctgcttattatgctcactattatcaacagcaagcacagccaccaccagcagcccctgcaggtgcaccaactacaactcaaactaatggacaaggagatcagcagaatccagccccagctggacaggttgattataccaaggcttgggaagagtactacaagaaaatgggtcaagcagttcctgctccgactggggctcctccaggtggtcagccagattatagtgcagcctgggctgagtattatagacaacaagcagcctattatgcccagacaagtccccagggaatgccacagcatcctccagcacctcaggtataa

SEQ ID NO: 255 - DNA, 26405 bp, Mus musculus
  source 1..26405: mol_type = other DNA
gctgaaacatcgcgggatttccgtctgccagtctccgcccctttacggcacgatggtcgtgcaagaatgtgatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcaggtaagcgtggaccgaccgcgcggcggcttcccctccccgacccccgagagccgccggcggggcaggtccggccctgctcgcactcgcggctgccgcccgaggagatggcgtcttctcacgaggggtcccagcggctcgaggccgagcccgttgtcgtggtctgaagcgcccccccctcccacgcgctctccccgagcgtggcctccacttccgccccgcgcgcgcggcctcgccgccccgtgcgccctgcctgccccggctccctcggctccggttccgcccgacccaggcgcgtggaaggttctgggcgctcaggtcacggcgagggcaagccagggagggccggctgcctggctagcggtccccgccgcttcccgcgccgccgagctctccaccctcgccgtaattagcctaacgttccccgcggcgttcaccagcgaggctctccccggggcgggatctgcgtgcgatccaccaatcctttccccagctccaaatggccccgggccctttcttccccagccttgagtttgaatctgcctctctcttcccaagtcgaagcctccaacctctttgtgctacgtagggacgtactgttgcctcgaatggcaaccattttcccagatgaaaagggttggatagataagagtccttgcatttgacaccccccccccaataacctgaacgtcaatgtcttcaagtagcccccgcccccgacttctttgtagcttaaaaatgtataatgttgccttgccgggtggggtggtgctcgactttagtcccagcactcgagagacagagcctggcaggtctcttgagtttgaggccagtcaaaacggccaagagggacctgtctcaaaaaaaaaagtatgttatggaatttgcctgtaaggtaaaataaacccttatggctctgttgtagttgctagacatgagcaggtttttaaagccagctttgactatactggaaacttccttcaggaccgaaagattaccgactcaatcaaggtttatcaagtctaccataatacagcaagcacacttatctagtgcatagatactgaaaaaatatatttattcatacagagggattccatgagcaatgtggttggtgtacatagaagtcagaggacaacttgtgggagttgattctccttccatgtaggaccctttggtcaggtgtaagttgttaggctcagggacaagtgtgtctgcctgggcatctgaggaaactttttgatgcagggcctctaccgaaagcctaggcaggcctgaggtttaaggcttcctgctgaattgtctctagtgtaggtgtactccacctctcggctttggaagctttttaagtgtgtgatttctagtcttgtgagataaaaatcgaacacgagtgagcagacagctttgatgataaagtaaaccacagcggttacataaatagtcacactgtgaggcaggcgctgtacaggacgctgctggaaagggatgcctcactgaaagatgggtgacattcatatagtgcagtgaggagtgggagtctggccagcatagacaagaagctgctgcttgctctacaaagatcgagggtttctggaaggacagctctaagaggtagtcagctaatataggataacataggaggaggaaaaaggtgggaatgtgagaaaaactatggatagattccattgttagcaagcattggctgtgataatacaagtgtttattatagatgatagtatctgttacacctttggacattctgggtatgtagaaattccatcaggaccttgcctgtgtaagtaagggactaaggtctcactatgtagttctggctgtcctggaactcactgcctacctctgcctttaggactaaaggtgtgtgccacccatgcccagatgagattttgttttcaaaatgagagtctttctgcctcaccctcctgaatgctggagtgacaggtctgcctacactgatcctggcacacttactttttctaaaaattattttctttatttacattcctgcccactcacccccagttcctcacaccattccttcttcatctccccacctcccttccctggggcctcaagtctcaagggttaggtggatcttctcccactgagaccagaccaggcagtcctctgctgttacacacacacacacacacacacacacacacacacatatacacacataagccagggaacttggaccagcccgagtgtgctgctaggcacactttttataagtttggtgcaactgtgtctttgaggatgaatgtgcgcattgttaaattttccaaaaggaaaatttgaaagttgctgtggaccctgcctaggaagcactagaagtagatttgcacttccacctttaaaactactaaagaaagtgtgggctaaagtgtaggagatagatgttttcacgtcattaccttcttagaattatctttaactccaaaattggtgagggtgggtcagtgaagaaagtggtaaatgctggtttggtttgatttttgtctgaagtaaaaaagcacagaaatacagtagagcaaaaatcaagtggaaagcttgaattatttctgtgggttaaaataaaaacattacttacacttaatgtgtgtttaatagaaacattgggttcataattgtagcatgtgagaagttaggattagactagctggggctataatcagaccatgttcccatccacccaccacccacccccaatgaggggagagttgaaagttttgcttggtcttttgaatgaactgtgtaccaatttttcttttggatgcttgtagacctattacatgaatggagtgtgaataagcatgctttcttatggatattccttggtcttcaagtaaactcagaagtgttcataaaagctaattgtagaacatagccatagtctaagccgttttcctctcgttcaattgtacgtaacacttcccttttaacatcagttcctgtcagtcccatacagtgcacttaaatacctaagatatagtttacaaatgctatgatttgaaatcaaagggtggaaaagattttgtttccaaatgtgtgggttgttttaaatggggatataacataagtttaatagatcattaactatttgttagctccctgaagtattagtggtagttttaattattcttaataagtgaatctttctagtgtttttaaaagtatatcttttattgtaggaaatttagtgctgagtagtacataaagtattaataccctgattagtattccgatttttcttttaagattgcagcaaaaattgggggtgatgctggtacatcattgaattcaaatgactatggttatgggggacaaaaaagacccttagaagatggaggtaagttatagccaacaagaataattatcttccatggtattcatttgataagattttcagtattgtttatcaagatgatttatcaaaatactactatctattggtctcagaagaataggattttgaaaaagacagacctagaaaaggtaggtataatttgaatctattgggaagtattattgtaatagaaaacaggttttgttatttttttttttttttgtcctgttataggattatgcttgctttgagttgcacagactgttaatgatataaataaagttatgttatttgggaaacactttactgaattttctcatttattgaatgagtctttgatactccctttcattcagtcagaatccacgtctaggttaaggttggttggttgcaggcatcccatttttatattgttttagtgttttgtgtgtttcatatttatctttaaacattgattaaaactgtggattcatagtgttttctctttttaaaaatcatcacttctgcacttagaagtgtgtacttttttattttagttttgatgtatgtataaattttgatattgatatatttgcccttttgtaaaagattctgatagcatataggactgactgtatttgtggccctttctggtaatacttggtttccttttgtttaagtatgtttatatttgcaagtgaggtttttaatatgcaagaaaaaatgagctaatgatgcttcagatgttgtgtgtaatgtactctttttattttatgtgtagatggctcttggacaaatccgagcagtaccacacactgggagggaatgccctctccttttaaaggcaggaatttttatttattacctgtgtttagtatgtaaatgtgaaataagccagtggattcttaaatggacacaatggtctcttggtttatgtgtctgaatttttgctatacaaaagccttgaagttgggagaataataatagtgttttgagctataggctgtctattcatatgttgtatgcattttgtttatttgagaaaggcaatcgtgtggttgctttgaggttttggtgttctgtagtgaaatgcttcatttgagaataatagaacagtttagattgaaattgtcttgtaacgtgagctttcttttttggtatagttttatgatcactgtcagattcactcatcttttgatatgtctggtttttatgtagtgctctctaattgtcagttctagagtggatgaagcatcattaaccattagcataaggtattcaccagcattgaagctaaacagtttgtgctgtattattccaaactaaagtgtctgtgcattgtttaaacacaagagaatgaaagaatttaggttttgtgggttttttttttttttttagatttttgagtctaggttgaattgtgtcagttttgattcaattggtaaaccaatatatcatgtattttaataaatatcattattcttgcttggtgggggtggggagctttgtttttaattgacaaaaagtaaaagacacttaattttgcagatcagccagatgctaagaaagtacctccccaaaatgactgtaagtactttaagctgaggttttgttgcttctttaaaaattacgtgtgtgtgtgtgtgtgtgtgtatgtgtgtgtgtatgtatgtatgtatgtatgtatgtcaagatggcataggaattccctgaaagtggactacatgtgggtgctgagaacttggttctctacaagagcatgtgtcctctttatcatctaagtcttctctccagactgttgtttctactttgggtgggttgtgagtttggggtttggggaatttgcttttgagccaaagtctcaaatatgtaattctgactgcccttgacttcacagagatctacctgtgtaaaagtattttttattttgcctgatgtaattgtcatctctgagccttactgcctcaaaccttctagttctttctgaactctggtgcaaactcctctccaagctgattcaatatagcttctctcttgacctctgccggaattgctctgcttggcctcaaactatccttctcattttctggcttattctgtttttacatgtgtctagcttgttccctctctgcaacctatatatctctacaactgttccaataagactgtcctcccaccccctgggctatttatttatctcttaagtcctcctctctattctcctgagagctagacataattaatcccattctttttggggtttttttttggggggggggggttagacagggtttctctgtgtagccctggctgtcctggaactcactctgtagaccaggctggcctcgaactcagaaatccacctgcctttgcctcccaagtgctgggattaaaggtgtgcgccaccaacgcccggctgtttgtttttcaaaacagggtttctctgtagctctggctgtctagaactcaccctggaccaggctgacctctgcctcccaagtgctgggattaaaaggcgtgacccaccactgcccggctttttttccttttcttttcctcaaagatttgttactttatatatgagtgcactgtagctgccttcaggcacactagaatcggatcctatcccattacagatggttgtgagccaccctgtggttgctgggaattgatctcaggacctctggaagaacagtcagtgctcttaaccactgagccattttcccagccctaattttattcttttgactccttctctgattgatcactttgtctgctactctaaattaaacatcactttcaaacatgagtgcttccttctacaaactaacttcatctttactgtttgggattaaaggtcattaaaggattaaagagcaaagccacaccacaactataagcacatacacccccacccccgtaaataacacaatcttggggctcaccctataatctaagtgatcaaatagcctacaacagacttgcttttgttttctcctgtgcttggactaaggatgtgtgtgccttgccatactcaacagggttggtattacaaatgtctagctggaatttccttacaaatgtatataggtttttactatatttttaaaaaagaatgattctcttttacttgggagccacgtatatacaaacactgatggctgagctgtgactataagttaactactctagttgttaatctgtgtgtccctttcctgactttcttgctactctcagtgatgattcaaatatgaatggtccgaatttttttcctatgtatacatcagggaaagagtttggagtacgtggtatgtttgtctcaaataaactcgaacttaagcttgtttttgttcattttttcgtagcttttggagcacagttacctccaatgcatcagcagcaaaggtatagtcaaaacttttaaagctactttgcaagtcttagttggattgtatgtacagccaagttagtctataatgcaaatgtctgcataccattttgtatgtgtggtttacttctgtaaaccacattgggaaaatggtatagaaacattgttaatttgggaaaacaggttgaaattgacgcatttcagaagaggtaaaataaatgttaaagtggcattcgctttgacaggtgttcagtttcctgccctgctcacacatgctgcctcatttaagaacctggctgggctactgggagactttaagattgggtttattagactgcttttatgtgtatatgtacattatttttctgtgaggaaaatgttagtttctgttctttcttagcagatctgtaatgacagaagaatacaaagtcccagatgggatggtcggatttagtaagtaccattaatactaaggttttaagacaaagcatactttcaaaaatagtatgaatgataacttctcccctttctctaccttaactagtaattggcagaggaggtgaacagatctcacgaatacagcaggaatctggatgcaagatacagatagcacctggtaacattccgtgtgtgtgtgtgtgtgtgtacgaattacttgttgttttgtccagttcttacagttttttgagagtggctaggaaactcaagtattttaagtttctaaatatctgatatttttattgtattttttacttaaacttttctttttatttaattgtagatagtggtggcctaccagaaaggtcttgtatgctaactggaacacctgaatctgtccagtaagtttgaaaatgttaagtctacttacagttaagactgaagactataagcttcgttttcataccatttcgctttaaaagtttacatattatgcctttcttaaaatagggttcatcatctgaacctaagacagagaaattgttaaagccagagactaagccgtaactcaggcatttaggtgcttgccttccatgtaggaagaactgagtttgatcctaaaactaaaaaaaaaaaaactggcgtggtaatatcatggctcaggagttggacacaggcagattttggggacttggtaaccaaccagctaacactatttgatgggttcaaagtcattgagagaccttgcctcaaggtacttatcacaaaaaaggtactgatggttgtaatttgacctatacaccttccatacgtgtctatacagactcagaaattattaaaacctgccttgcacaaatatgttaataacgtcacctaaaactcatgaccaatctagtcatgaaaattttatattcctgtttttctcttccactcaattctaaggctctgataaaactacccaatctttacctctaattgtcttctccccactgccatctcccctccagacagggtttcattgtatatccatagctgactgtctctgaactcactcgctctgtagtccaggctggcctaaaacttaaaagatctgcctgccgaaggcatgtaccaccattgcccaccacactcactgtcttgactgacttgtgttgctgtgaccaagaaagtgttatcatcctacactgaaatcttaacaccgttccttgcatacttttttttttttgtctagtattgagatactgttttacgttcatttgcctgactttctacttcaagaaattcctgactaaactgattttttacttcattcctaaaattttaggagtaatttctaagagtttatagacagatttggaaattacatatcataaaaatatctttgaacctcatgttgtattttgttaacacagtgctttaaaaggtttgtaccattgaagtttcccctttaaaagtttatctagtgtgtgtgcctctcagtttccttcaagctctgcttctaacagctgcatcgacagttctctttccaaggcagaggctgagtcgtgcactagttaaccccgttgctgtgtgacaggatggatggaagatgtttaatgactgtgttcacttggagcatacagacttcagtacttactgcccatttgacttgacactcactcctttggtttaggttcttgcctccgaccatgaaatgccatatgaccaatttagagtatgtttgaaacctgactaagataaaactgctgacttttattattttgggataagttgggttatgctacgtatttttatcaagattaatttgttaacagatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggtattattatattgtaaagaatattattaagtcttttgaggccactttttattgtttttactaacatattttcatgatattaataggaacgggctggtgttaaaatggtaatgattcaagatgggcctcaaaacactggtgctgataaacctcttaggattacgggtgacccatacaaagttcaggtaagcttcactttgtctcttatatataaaggctgagggggtggacaaaatacgtattaaatttttttgacaaatattctgaattggataactttctattatagcaagccaaggaaatggtattagagttaattcgtgatcaaggtggtttcagagaagtgcggaatgagtatggctcaagaataggaggcaatgaagggatagatgtaagtaagaattctgagtcagcagtggttgtatcctaatccatgggcactagtgttttcctgctttgaattaaatggacttggcttaacatcatgtcccttttcttgttttcttccttggggtttttttatcttgttatttttatgatcatttagtatatttacttttatgattaagtggggatgagtttttgagaaatgtcttttaaaatatacttttacttaaggtcccaattccaagatttgctgttggcattgtaataggaagaaatggagaaatgattaaaaaaatacaaaatgatgctggtgttcgaattcagtttaagccaggtaagtttaaggtatatttaatgttctaagaattgatagcttttaatcttgacatttataattggtttgtcacaacttactctgtttttttttgttgttgttgttgttattgttttagatgatggaacaacacctgataggatagcacagataacaggacctccagacaggtgtcagcacgctgcagaaataatcacagaccttctacgaagtgttcaggtgtgataggaaattaacattttcagtttgctttctttggaaaagtttttatcatacctgaatgaaaaaaatgcccctccaggttatattagaatctgttggcagtgttaaaatgactaacgttaaattgctttaaggtaaacattttagggtagatttgcataaatgttctgccttatttcccccagtgcatgtgaacttttatttctatgggtatatgtgccttggcaattgtgcctatggaagccaaaggaaatttttctgaagttggttctctattatgtgggtcctagaaattaaacccaggtcattttgcatgtatgggtagtctcccaatatattttaaaagtggaagttattacttagttgactatttttgtttgacgtcatctcttaatttcattgtttagaaatacgagagttggggataataagctaatagttttattacagagtttattttgttctctcctttcttaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggcaatgtattttaaactctttgttttgatatactggatatttgtaaacaaaagatgctatttatatttttttaggaaaggggtcttaacatggcagctaaaataagaatactctttaatgtatgctaaaaatgggattgtgtctcaactgcttctagggaaaggggaccaaaagtctgtcagttacaaaacttgatcctaattcaaatgggttttgttggtgttttttgttttgttttgcatttaacttctttgttacttacaaaacctgttgattgaaatatttttctgttctgacccatatcagggtgtatgtttagtaaggtgtgtataaaatagactttagtgtgttttgcattagttctagatggaagaacaggctaaatagaaaatcctaacattattgtccagtggaatgctacaaaactgtttaatgggcaaagtaataggtgtttggggttttttttggttttttttaaatagtatttctgccagacatggtgacagacacctttaatcccagcacttagtaggcagatgcaggtgtttctcttgtgagtttgaagccagcatggtctacagggtgagttataggacaaccagggccacacagagacccttgtcttaggaaagagtattgcttgcattctgtgaacttagtgtcttgcggttttaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattggggtaagtgtactctcatttatagcatagttcctggtaactttaaaatgtactgtgggaaaatacatcgtttctaatatgaaattgcaggtattgtgatatagtgtatatacatatattgacataggaaagatatccaaaatacaagaaccatgaactcagaaatccgcctgcctctgcctgtcaagtgctgggattaaaggcgtgcgccaccacacccggctgagaataccatgtgttaagttttcccttgatgtgcaatttgcagaatatactctcagcagttgtcacattagggaagtaaagggaactaggcagtcttgtttttaaaagaaaatcataaaaagagtaagaaacacagaagactaagctgaaagaactctgctggagctgaggatttcagtgaattttacatggcagatgatgacattttattttttgcaccaaaattcaaagagggtaaatttaaatttgttatttaagcgtagaaatggaagagtagttggactttgaattactcaaccattacattgtttgagtgtctagttattgtttatgttttcttttcctttttttttttataatagggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcagtaagtaccacatttgcttggttctgggctttccctaaagacctaggaattgggggctggagagatgacgatggctcagcagtgcagagcacttgttcctgttgcagagggacctgagctcacttcccagcaccacacgggggttcgcaaccattcgtgacaggttccagggtatctttacagcgacactaggcatacatagctgcatatacatacatgcaatacatgcagaggcaaaacctacacataaaataaataaagctggcttttattaatggtatttgtgttttgttccccacccctagcggtcctccagccccatatgctccccagggatggggaaatgcgtatccacattggcagcaacaggctcctcctgacccaggtaaagggtaatctactattaatatgttagcttcattgtatactgctggtcacagtcctaaactacctactgcatgtgcgatcttttaaggcacttcccctgcagtataggatgacactgctgtctagtatgttgtgactttattcatagtagttcatatttataaaataatgtgtttaacactactatgttctcaaataataagtataaattattttgttgctgataaaattaaggtcccttaattaccaaaacaggagagtacatttatttttctctcttataatctggattttctatagcatggtgttctcattatatcttcccatactggttttgtttctactccatcatgatcaggaatcaacttggaaataggacttttttctttgtttttcttttgttttttcactttgcagcccctgtagtttgattcatttgaaaagtttgagagaacgtgttctgagttatagatttctacatagtgtattttttacaaatcaaagttgatttagatgtaattgctttgttggatttgttaaatgttttaatattttaatgtatgttaaattcataggaacttttctcagtcaccaaataaccacaacttctattcatttaagaaacaacttgagccgggcggcagtggtgaacgccattagtcccagcacacaggagacagaagcaggcagatctctgagttcgaggacagcctgatctacagagcatgttcaaggatagccagggacacacaggaaaaatcctatctcaaaaaagagagagagagaaaaaaaaagaaaaagaaataacttgaattgtgatggcatctgaatatgaaagatgagtgtcgattagcaaaagagtgcattcccagggcagacaagaagcatcagttatgacttcctttgtaagggttttaaaagtgaatcttctccttctaacaagcatttagacgtggcatgatcaaatagagttggaacaggttatattgaaggttttatatggctaattcaatttcataggattgctcaaatgagtgatgtttgatattagcactgtaaggtctggtcagtgaatatgacttccagcattacctggaagactgactcggcaatatgttgcttaaatttgacttggaagtatacagtattttattgtctgcattttcaagaaactgctgctgtgaggttgagatttatatagatgtcaagaatttaatcttcaatcttaaatggataatatgcgaattcatcctatgtaaaagaatagtgagctcttagtacctgctagttgcaagtcacaaaaaaaaagtttgggttttggtttggtttttgagttacaaaggtgagcttagtgcagaggatgctggttgtggtgacttgggaatagggcagcagctaaaacttgagtattgggggatgctaaaagaattaatagaaaggataagccacagtgctggggataagcattagaacctgagttctatccctcccacctgttgtaaagaaaaataaataaagcgctttaataaagttcgtaaaaaggatccgtaaagtttcctggcctgctcttgtaaccaaatctaggagctctgggttcagagagaaatcctatctcaaaaaaaattaggaaggaaggctggagagagggctcagtggataagaaaagcaataactgctcttccagaggtctgagttcaattcccagcaaccacatggtagttcacaaccatctgcaatgggatctgatgccctctgctggtgtttctgaagagagcttcaagtgtactcatatacaaaataataataattcttttaaaaaatgaggaagggtgtcagagaggtagcttagtgattattataaaggtcttcctgctctttgagaggatgcaagttgagatccagttactctcacaccttctggttctctttagtaacccacatttgtgtagatacacacaggttcacagatacacataactagaagtgggactttaaagtctagataaaacaaggtgagcatgtctagggagatcacccagaatcagtacacacaaacagacacgccacgacacgatggctaagctggggagggcggccagaccacagcatgtagcccggaaacaggaaaagtggaggaagaaaaggtaacggcttggaaatccaacggcctgtgttaaatggggcatggtcttaaaactgtacaggagctttcaaatgataagctatgcttgaagtatcctaaaggtgttagaaaaggatagagcaggactttcaggattgtgactgaaataaatataaactgcatgtatctaacctttgtttctttcttgtacaaattagtccataactgcctgctatacagaactgttgcacttggcagcttaacttgttgggggaaaaaaagcaaagacacgggattgagctgagacaaaccattggattggaagccaaaagatcatccatttatataccagttccaccgatcactagtaaattacttaatacctgtaacctaggaaattacttaattcccgaggctgaagtttctgaactaaaagccaagtgatgatgataatactagaacattttgaaagtttccattcgctattggtaacttttgagcctatttgtcaggaatcaccaaaaaggtaagggttggtttctagagtttacatattaaccttgaaaccgctctttgtttttacattgtagccaaggcaggagcagatccaaactcggcagcttgggctgcttattatgctcactattaccagcagcaggcacaacccccacctgcagctcctgccggtgcaccagctacaacccaaacgaacggacaaggtaactacggtaatgaaagattttatttcctattttaaagctatagtttgtgtttggattcatgtatgaagtcataacaaaactctctttcagtcctgagaattgttgggccttgtgcatgatggtatcattaggttagatcctcgggcctaaggttttgaagactcagttgtgactttgtgtaagcaaactaagtcaaataatagttggaagaataatgccttaagtagtgagactgttaactgtcctccccccacccccatttacttcactaccctgatttatgtctgttataatagttctgaagagagctactgaaaactgtacttagagacataaaatcatatcacctttctacagacccttattcattccaaaataaatgaccaccatggactttaaagtcagttactcatcattgatataccactgttatacacagcccttgcttagatcaccaactgaccccgtgttttcattaggttcattaagtctcatcaagctagagcactttcttggtcttcttttatgcctctaataagaggataactcaacagttagatagctgccaagctatagagttactaaccatggttgtttaaatggtcttgctactaccagcctggtttttgcactatactacaactatgtactgtaaagaattacctaggggctggtgagatggctcagtggataagagcacccgactgctcttccaaaggtccggagttcaaatcccagcaaccacatggtggctcacaaccatctgtaatgggatacgatccgattctagtgtgcctgaaggcagctacagtgtacttacatataataaataaataaatcttttaaaaaaaaaaagaattacctagtattttctgctactttttactggagtaatgtcactatcctatacattgaacttctgcctcagccttagtctgtattattaataaaccttgccttggtaccctactattacagcagctgttaactgttagtttctgtcattgtatattagttgctattttactacaaggaatagattgcccttttttaactactttcattatattgtcctagatttggggtggttggggttatcttcaaggtggctctggtgcctctgacgttcgttcttcaggtgcttccttagtacctgacacagtaagataactttgtcttctgagctccaatcggtgattcatttccttttctctctctctttctctctctctctctctctctctctctccctctctctctctctctctctctctctctctctcaggatagaatttagagatcagtctaagtatactaattgctatctaaatgtcactaattataatcctgtccaaattagaactaaaaagtatgtttggggctggcattcagtcaatagtacttgttcctgcagaaaacccaggtttgattgctagtacccacatggtaactcacactgaaactgattccaggggacccagtgccctcttctgatctgtggagcaccagatacccagggtgcacagattatacatgtggacaaaatgctcacacataaaattaaacagtatctttaaaaacatgcaggacaacacgtgtctactaaaaatcacatgattatatactactttttcatttaatctttaaaattagtgcttcaaagctatttgtggcttttctaaaaatgtgtgcaggagatatcaagggatgaaccatgcttgaactgtccttaaaaatggatagaaaccatgttctatatttcttttgtgtgcatttacttactattcccagtttctgtgtccaccccttctatacccacttgtcatgtattcccaacaaacacttgcacaactccgtggtgtagacagtgctaattacagtactgatggtgtagaagcaaaagtgcataacaaccttcagaaacttaaatcttgttttgtttgtttgttttttaaaaaggagatcagcaggctccagctccagctggacaggttgattatacaaaggcttgggaagaatactacaagaaaatgggtatgctctacagtttacgtcttatcctttatcttagtaactaaacggaagtaaatacacattacaaagctagagatgagtaacaaaacatttgcttgtttttaaaaaagaatacttgaggtaactcatccgtatgtttttataatgttaaagtttatattaaacaagctaggcagtggtgatacctgcctttaatcccagtacttggaaggtagaggaaggtagatctccgagtttgaggccagccttgtctacaaagcaagttccaggacacttgggactcttagacagagaaaccccatcttgaaaaacaaaaataataataaacaggaaaggtgacatcctgaaattgactggaagttaagacatattaagagcaattcaagaaattcttataattgtgcttatggcagagaaatacaattgtcagcaagttaataattaaaacctggcatttccttactaggtaataatatatggctttattactgtacttttgtgtagtggtctaaatggaatgagattaaattttaggccagcaaagtagttgtatcactggctttactgcaaaataagcataaaattagcccacagtgacattgctttacatttctaaacatttgcatgcattgaaaaggaaaaaagggtgaccttttaaaaagattttgacaactattgaacgtaattcttaacataaccatagtgattgtgttaatgtatttcttgtcggtgatacagctttcgacaaagttcctttcagaacaactgtttggggaaatgtgataacaaacttctgcagtgaacagatgggctgataacttttcttaacctcactgatcgcaggcaggttctaacagatggaccttagcactagaaacttgaaacctgccgggcgtggtggcacacacctttaatcccagcacttggtgggcagaggcaggcagatttctgagttcgaggacagcctggtctacagagtgagttccatccaggacagccagggctacacagagaaaccctgtttggggcctggggggaagaaacttgaaaccttaaaacaatattgtgtgtaattagaatgcttacataatttcttctctgaactgtgatatttttgagagtagatggaaatgtattaatcattccgtggtagccgtgctgaaatagtgctacttgtttataagaagctgagcatgccaaggtgaaagtgcgctaactccattgtttgtttttactgtgtgagtgttgattgggtagaaacctggtaactggactacgtaattaagacagagtaaggtatttgtctgtgctcgattatgtcatgttatttgtttacatgacttcaaataagttttatagatattttgcattctgtattcttaaaaagtatattgactaggacttttgagagtaggtattttaactcaattaggttttagtttttaatattctagcactttccagcacacagttctgtgaattcctcttcttctaattattattattattattagagatttatttattttatgtatatgaatgcactgtagctgtacagatggttgtgagccttcatatggttgttggaaattgaattttaagaactctgctcaatccgacccaaagatttatttactgtcataaataagtacaccgttgctgacttcagacataccagaagagggcatcatatctcactatgggtggttgtgagctaccatgtggttgctaggatttgaactcaggaccttgagaagagtagtactcttactcgctgagccgtctcccagcccgaattcctgttcttggttgttatgtctaccgatcaatcagttggcatacatagagacagacacacacagacagcagcatcgtgcaggataactatttggagactcattttcttcatttgtgcttttttgtaattatgttgaagatcttaatcctgtgagcaaaaaaagactaactgaacaattccctgtcatatagatcttgaaagatactgttttaggtaatttaattaatcattgaattagtatttttaattatatgatagttataaaaatcatttttccctgtaaaacatttaactacaatttttttctgaaagtatggtccttgatatattttttagtataatcatgaattaagagaccttaaagcaaaaggttcagaatagaagctcaggagatagatgggtaagagtgcttaatgacctgggttcaaatcccactacctatcttaaaagccaatcatgactgttgcatgttgggaagcagttgcaggagcttgctagccaactgtggattcagtaaaaggagcaggggaataaggtacagagggatagaagaaggcaaaggcaggcagatttctgagttcgaggccagcctggtctgcaaagtgagttccaggacagccagggctatacagagaaaccctgtctcgaaaaaaaagaaaaagaaactcatttgcagttaacttgcttgtttacaaaattcattaagtatttctagaattcgatttttatcatttgataactcagaatggtcacttaaaacaaaattactaataaacgctgagttttgggctggtgagatcgctcagtgggtaagagcaccctactgctcgtccgaaggtccggagttcaaatcccagcaaccacatggtggctcacaacaaccatctgtaacaagatctgacaccctcttctggagtgtctgaagacagctacagtgtacttacatataataaataaataaatctttaaaaaaaaaaagctgagttttacatctttcattgaactttatttcagctgccaggttatttttagtaaagctgtgatataaactcagtatgtcttttgcttcattgtgtgtgtcataaatatctttcagtacttctctgtagtttaagactagtaatatagtgacccaaatggatagaagagtggtgagctaggtatggtggcacatatacgcatttagtcctagtgctcaggaggcagaagcaggtagatctccaaggatgaggtcagccctggatagtgagaccctttctcagttagcaaaaaaaaaaaacaaagcgtttcttctttccttgtcaaagattatatctgaattttcccctttgtttaaaggtatgtgagtttgggtcattttgtcttttactaatcaagctgaactaagagactgaaacttgtgattgaagtgaaaagagtatctttgccgtttcttgtgtgctgcactttcccccctcatcctccctcccttgtctaaggcatatttaagataaacagaaactgaaggggcaatgaggcacagataatcctagcacgtgggaagttgccaagaggataacaagttttgagccacctgtgctacataagtgaaaccctgtgttgagataaaaacaagagctattgcccctctgtgccttgcattataagctgtacttaatggtacatacatttttgtattaaagttgagggggtgttttaggtcacaagggcagacacgaggttattaaaaggcttggaagaagtattaaaaaggcaagatgctgtgtttactagagaagagttactgtgagatttgtaattatagcagcaataaagttctctgttttcctgaaggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcaggtagaaaacatttgctattttttttgttttgtttttgtttttgttttccactccaaccatgttttctgcatgtttgtctgtttgggaatgaatatgttttacttggtaaagtataactacatgaaatatcgtgttgtgtttggggactcagtttttctaataatgttttctggcatctgagtgctatttttgtaatgcctttgattttaaaaataaattttcttccccccagggatttgcaaatcatgcaagaagccaccatcatttatattaaccagtttttctttcttaaaggattcactcctgaattagctccatttaaaggattttctttaactttttgtgtatctcttatgtatctcttctgcacagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcctgtgtcggtaagttggatatgtgggtaaaggattcatagtttcttagcagtgataaattaagatacatgtacacgaatatataagctttccccatgaattactgagtttttaaacactggcatgttttttcccctgttggagtatagtggtagattggaggttcttttctgttgtatttggctatttcagcacaagtaatcctgatattttcatgtttttccttctatttgattaaaaactgcatgtgtatacaatgatctttagtatacttccattgcattaacagtgacatttccttttatacatgagcactatttcagacctgtaccgctgctacaacagttaaccttcctgttcttcacttatttccgagactgtttcagcataactaattttgaacagtttgcagacagtgatttgaggagtttataagaaacattgtttttttcatgtaaagcaactctttccatgtatatatatatattataatagtgtgtttctctctaaattcaggatagaaaagtaatagaatgtgaaagtatagctacattgcatctgccattgaaacatttggggtatgaaaatgttcaagctttttttttctttttgcagtatagataagctttgtcttgtaattgcacaagtccagtcattgaatcaaattaatttttttatgtactgaatcattttattaatctttaacattcatgctgaagttctgatattttgttgaaaaccattgttttactctgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagagtgacaaattgatgaactgcaacagtgtctttttgtcgatgttccacataatgcaattgctatattttgtgtgactattatgttggaatacagtgctgtcatgggaaaccataactgcttcttaacatagaataatacatagttctgtattttttttaagtgagcttaatgggtaagtatttttgatatgctttagctaatagctaaagaaaattgatcagtaacaaagttgaatagtattatcagtgctcctaaaatgctgtttttcagtgtaaaatatgcctatcttttatagtgatatgaaaaacttgaaatgtttaagacagaagtgcttttcagtttgcaaagtttaaggacttaacgccttttcattaaatgttagttctatcatctgtggggagcaccatttgtatgaggacaaaaatgttgaggttatagggcagaaaatagtacagctcattgtggatgttttgaaatgttttttgattgttttatgtaacttagtgacttcccttccccttatttagatctggatgcatagctctagtatgaagtttaatagtaatagttaaggccttacctgttaagatcttaagtgtagggtattgacatgaacagtttcagtattttgcatgatattgttgcatagatgacctaggaaagtgttgtggtgcatttagtaattaaactgaaggaaatagttggattcagtatcataattcacaaattggaggctgttgattttgattcgtttaaaatttaaaatctttattaattgcaaacagtgcaattatttatacttcacagtgccttcccagaccttccaccttaggttctgctgcaaaaagcaacaggtaagcacaacctaaggccatctctatataaatatatcagtacatacatgttgtccctgtgaggtttgtggttgtgtactcactcaagcaatctgctgctgccgctgccccaaatgtactttgttatttatggtaccattctagtggaaagtctgttaagttgttcaagcaactgtttacaattttgggtgatgttttgtttttggtttttttgggttttttttttccccattaaaatgagtagattgctgcaataactgaaaaacatccaaatctttttgtgcacccctcctctcccagagttatagaaatgtttaagaacacttcagtagttgagacataggaaatcatttggtcagaaatttcaacagccttcacagcccgtttgtatgagtaacaggaaattctctttgccctccaaatctgatctttttatagttttatttttattatttttcccctgtattagtactgctttaaagtacagtaattcagtgagatcgttttggttcagtttcatagacagttctattttcatgacttgtatttgctgacaaggaagaataaagaatactggcagtaagttttgggaaaggcatgtgcatcagtgaaatgttacttgtataacaaataacctttcataatctgcataaccagtagtttctgatttataatattttaataactgactgcatttatttttgccagtttaaaatgttttgtgttcttaggattgatgtttagtgcatattttgagttaaatgactatcttaaagcagcaccatatcagttgtttttattcattttctaaaatgtgctgatcctattaaaaactcctgcttatcttttacaacaaagaaaaatattcaaaaatactgccttcattttcacacacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgactgtgttcccatacataatatggtatagggtgtaatgtacctgctcttgatcactttcattttaaagtgctattcacttaaatgttccatgaattgtttattgcaccacagtttgtgtgtgtgtgtgtgtgtgagagagagagagagagagagagaaataggtcagatcagtatgttgcaagcagatctttagagcctgtcaagttttggttagttgtagtttccatttggaaatgtagatgaatgcttgtagatattggagattgtttctatcttgtaagaacttttcactggtgctgtaagcatttcaaatagcaccagtcttaacctttaaatgggaagtagaaaaggtgagcccaaagtttacagatgattttaatgctatacatgttagtgtagtgatacttagaatgctttgtttgatgtttatttcagaaatgcgtatactagaaaatcattttaatattaaaactggtgacttaatactagttgtaaagtgttttcttaaagaaggatcttggtacttaattgataaagtgggtttagataggagtagcaagtgctctcgatagagaaagtttttgttcacttcaatactttgtcattacaaccagttcttcctgaaaatagttacatgtctagtaaattgatgtagaattaactcgctggagataatctcattttagttctgcaaattctgcctggctttaaaatgcattttcataatacttagaaataatttgactgaaaataactgcttttttatttaatcagtcaatcaacttttactacaaatttaattgagggattttttaatttaattggtgctttaaagaagcaaataaatccctgggttttgttttcttcagtaaatatcctaaagaaactctttaatgtatttgcgagtatatatatattttcttatgcatgctcgatgcattttcgtcctgagaaaagtgttctctacagaaactacccgtgtgttaaaagaagattggcttaaaatggctactgtgatgggaacagtgtcttagggagatgcagcttggacttgaggtaaattgaatactttacaaactggcttagagttttgctttaatgtcattatatgtaaaagggcacatgattattgtaattttgtattctttatggtttccttaattaaaataataaatgtacagtgattact

SEQ ID NO: 256 - DNA, 4525 bp, Mus musculus
  source 1..4525: mol_type = other DNA
cctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggtaactacggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcctgtgtcggtaagttggatatgtgggtaaaggattcatagtttcttagcagtgataaattaagatacatgtacacgaatatataagctttccccatgaattactgagtttttaaacactggcatgttttttcccctgttggagtatagtggtagattggaggttcttttctgttgtatttggctatttcagcacaagtaatcctgatattttcatgtttttccttctatttgattaaaaactgcatgtgtatacaatgatctttagtatacttccattgcattaacagtgacatttccttttatacatgagcactatttcagacctgtaccgctgctacaacagttaaccttcctgttcttcacttatttccgagactgtttcagcataactaattttgaacagtttgcagacagtgatttgaggagtttataagaaacattgtttttttcatgtaaagcaactctttccatgtatatatatatattataatagtgtgtttctctctaaattcaggatagaaaagtaatagaatgtgaaagtatagctacattgcatctgccattgaaacatttggggtatgaaaatgttcaagctttttttttctttttgcagtatagataagctttgtcttgtaattgcacaagtccagtcattgaatcaaattaatttttttatgtactgaatcattttattaatctttaacattcatgctgaagttctgatattttgttgaaaaccattgttttactctgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagagtgacaaattgatgaactgcaacagtgtctttttgtcgatgttccacataatgcaattgctatattttgtgtgactattatgttggaatacagtgctgtcatgggaaaccataactgcttcttaacatagaataatacatagttctgtattttttttaagtgagcttaatgggtaagtatttttgatatgctttagctaatagctaaagaaaattgatcagtaacaaagttgaatagtattatcagtgctcctaaaatgctgtttttcagtgtaaaatatgcctatcttttatagtgatatgaaaaacttgaaatgtttaagacagaagtgcttttcagtttgcaaagtttaaggacttaacgccttttcattaaatgttagttctatcatctgtggggagcaccatttgtatgaggacaaaaatgttgaggttatagggcagaaaatagtacagctcattgtggatgttttgaaatgttttttgattgttttatgtaacttagtgacttcccttccccttatttagatctggatgcatagctctagtatgaagtttaatagtaatagttaaggccttacctgttaagatcttaagtgtagggtattgacatgaacagtttcagtattttgcatgatattgttgcatagatgacctaggaaagtgttgtggtgcatttagtaattaaactgaaggaaatagttggattcagtatcataattcacaaattggaggctgttgattttgattcgtttaaaatttaaaatctttattaattgcaaacagtgcaattatttatacttcacagtgccttcccagaccttccaccttaggttctgctgcaaaaagcaacagtgctgaagatgctgcaagcaccaaatcatagctcataaaatcaggtcctgagatagttacccataaagaggaatcctttgagtgtatgccattggtgagccgatgagcatggaccatagaagggctcaatgtagaagaaactacccgtgtgttaaaagaagattggcttaaaatggctactgtgatgggaacagtgtcttagggagatgcagcttggacttgaggtaaattgaatactttacaaactggcttagagttttgctttaatgtcattatatgtaaaagggcacatgattattgtaattttgtattctttatggtttccttaattaaaataataaatgtacagtgattact

SEQ ID NO: 257 - DNA, 800 bp, Mus musculus
  source 1..800: mol_type = other DNA
cagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcctgtgtcggtaagttgga

SEQ ID NO: 258 - DNA, 2526 bp, Mus musculus
  source 1..2526: mol_type = other DNA
gcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagcagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggatttgcaaatcatgcaagaagccaccatcatttatattaaccagtttttctttcttaaaggattcactcctgaattagctccatttaaaggattttctttaactttttgtgtatctcttatgtatctcttctgcacagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctc

SEQ ID NO: 259 - DNA, 809 bp, Mus musculus
  source 1..809: mol_type = other DNA
gaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagtgccttcccagaccttccaccttaggttctgctgcaaaaagcaacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgactgtgttcccatacataatatggtatagggtgtaatgtacctgctcttgatcactttcattttaaagtgctattcacttaaatgttccatgaattgtttattgcaccacagtttgtgtgtgtgtgtgtgtgtgagagagagagagagagagagagaaataggtcagatcagtatgttgcaagcagatctttagagcc

SEQ ID NO: 260 - DNA, 1040 bp, Mus musculus
  source 1..1040: mol_type = other DNA
gatggtcgtgcaagaatgtgatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatggctcttggacaaatccgagcagtaccacacactgggagggaatgccctctccttttaaagatcagccagatgctaagaaagtacctccccaaaatgactcttttggagcacagttacctccaatgcatcagcagcaaagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaat

SEQ ID NO: 261 - DNA, 796 bp, Mus musculus
  source 1..796: mol_type = other DNA
cccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcc

SEQ ID NO: 262 - DNA, 585 bp, Mus musculus
  source 1..585: mol_type = other DNA
aatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagaaacaacttgagccgggcggcagtggtgaacgccattagtcccagcacacaggagacagaagcaggcagatctctgagttcgaggacagcctgatctacagagcatgttcaaggatagccagggacacacaggaaaaatcctatct

SEQ ID NO: 263 - DNA, 2374 bp, Mus musculus
  source 1..2374: mol_type = other DNA
ttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagcagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggtaactacggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctc

SEQ ID NO: 264 - DNA, 3163 bp, Mus musculus
  source 1..3163: mol_type = other DNA
gatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccaggtaaagggtaatctactattaatatgttagcttcattgtatactgctggtcacagtcctaaactacctactgcatgtgcgatcttttaaggcacttcccctgcagtataggatgacactgctgtctagtatgttgtgactttattcatagtagttcatatttataaaataatgtgtttaacactactatgttctcaaataataagtataaattattttgttgctgataaaattaaggtcccttaattaccaaaacaggagagtacatttatttttctctcttataatctggattttctatagcatggtgttctcattatatcttcccatactggttttgtttctactccatcatgatcaggaatcaacttggaaataggacttttttctttgtttttcttttgttttttcactttgcagcccctgtagtttgattcatttgaaaagtttgagagaacgtgttctgagttatagatttctacatagtgtattttttacaaatcaaagttgatttagatgtaattgctttgttggatttgttaaatgttttaatattttaatgtatgttaaattcataggaacttttctcagtcaccaaataaccacaacttctattcatttaagaaacaacttgagccgggcggcagtggtgaacgccattagtcccagcacacaggagacagaagcaggcagatctctgagttcgaggacagcctgatctacagagcatgttcaaggatagccagggacacacaggaaaaatcctatctcaaaaaagagagagagagaaaaaaaaagaaaaagaaataacttgaattgtgatggcatctgaatatgaaagatgagtgtcgattagcaaaagagtgcattcccagggcagacaagaagcatcagttatgacttcctttgtaagggttttaaaagtgaatcttctccttctaacaagcatttagacgtggcatgatcaaatagagttggaacaggttatattgaaggttttatatggctaattcaatttcataggattgctcaaatgagtgatgtttgatattagcactgtaaggtctggtcagtgaatatgacttccagcattacctggaagactgactcggcaatatgttgcttaaatttgacttggaagtatacagtattttattgtctgcattttcaagaaactgctgctgtgaggttgagatttatatagatgtcaagaatttaatcttcaatcttaaatggataatatgcgaattcatcctatgtaaaagaatagtgagctcttagtacctgctagttgcaagtcacaaaaaaaaagtttgggttttggtttggtttttgagttacaaaggtgagcttagtgcagaggatgctggttgtggtgacttgggaatagggcagcagctaaaacttgagtattgggggatgctaaaagaattaatagaaaggataagccacagtgctggggataagcattagaacctgagttctatccctcccacctgttg

SEQ ID NO: 265 - DNA, 6523 bp, Mus musculus
  source 1..6523: mol_type = other DNA
gctgaaacatcgcgggatttccgtctgccagtctccgcccctttacggcacgatggtcgtgcaagaatgtgatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggtaactacggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcctgtgtcggtaagttggatatgtgggtaaaggattcatagtttcttagcagtgataaattaagatacatgtacacgaatatataagctttccccatgaattactgagtttttaaacactggcatgttttttcccctgttggagtatagtggtagattggaggttcttttctgttgtatttggctatttcagcacaagtaatcctgatattttcatgtttttccttctatttgattaaaaactgcatgtgtatacaatgatctttagtatacttccattgcattaacagtgacatttccttttatacatgagcactatttcagacctgtaccgctgctacaacagttaaccttcctgttcttcacttatttccgagactgtttcagcataactaattttgaacagtttgcagacagtgatttgaggagtttataagaaacattgtttttttcatgtaaagcaactctttccatgtatatatatatattataatagtgtgtttctctctaaattcaggatagaaaagtaatagaatgtgaaagtatagctacattgcatctgccattgaaacatttggggtatgaaaatgttcaagctttttttttctttttgcagtatagataagctttgtcttgtaattgcacaagtccagtcattgaatcaaattaatttttttatgtactgaatcattttattaatctttaacattcatgctgaagttctgatattttgttgaaaaccattgttttactctgcatatttgttggctctttgcatattaatatattagactacatgcaaatacagtctgtcttgccattgtctgttgaagtgcaggtttgatccagccagtatagaactagctctgtaggggtgaggaggactgtgctgtgtatcatccttgattgtgttccttcaaggagcattgcactgtaagtacatcagagtgacaaattgatgaactgcaacagtgtctttttgtcgatgttccacataatgcaattgctatattttgtgtgactattatgttggaatacagtgctgtcatgggaaaccataactgcttcttaacatagaataatacatagttctgtattttttttaagtgagcttaatgggtaagtatttttgatatgctttagctaatagctaaagaaaattgatcagtaacaaagttgaatagtattatcagtgctcctaaaatgctgtttttcagtgtaaaatatgcctatcttttatagtgatatgaaaaacttgaaatgtttaagacagaagtgcttttcagtttgcaaagtttaaggacttaacgccttttcattaaatgttagttctatcatctgtggggagcaccatttgtatgaggacaaaaatgttgaggttatagggcagaaaatagtacagctcattgtggatgttttgaaatgttttttgattgttttatgtaacttagtgacttcccttccccttatttagatctggatgcatagctctagtatgaagtttaatagtaatagttaaggccttacctgttaagatcttaagtgtagggtattgacatgaacagtttcagtattttgcatgatattgttgcatagatgacctaggaaagtgttgtggtgcatttagtaattaaactgaaggaaatagttggattcagtatcataattcacaaattggaggctgttgattttgattcgtttaaaatttaaaatctttattaattgcaaacagtgcaattatttatacttcacagtgccttcccagaccttccaccttaggttctgctgcaaaaagcaacaggtaagcacaacctaaggccatctctatataaatatatcagtacatacatgttgtccctgtgaggtttgtggttgtgtactcactcaagcaatctgctgctgccgctgccccaaatgtactttgttatttatggtaccattctagtggaaagtctgttaagttgttcaagcaactgtttacaattttgggtgatgttttgtttttggtttttttgggttttttttttccccattaaaatgagtagattgctgcaataactgaaaaacatccaaatctttttgtgcacccctcctctcccagagttatagaaatgtttaagaacacttcagtagttgagacataggaaatcatttggtcagaaatttcaacagccttcacagcccgtttgtatgagtaacaggaaattctctttgccctccaaatctgatctttttatagttttatttttattatttttcccctgtattagtactgctttaaagtacagtaattcagtgagatcgttttggttcagtttcatagacagttctattttcatgacttgtatttgctgacaaggaagaataaagaatactggcagtaagttttgggaaaggcatgtgcatcagtgaaatgttacttgtataacaaataacctttcataatctgcataaccagtagtttctgatttataatattttaataactgactgcatttatttttgccagtttaaaatgttttgtgttcttaggattgatgtttagtgcatattttgagttaaatgactatcttaaagcagcaccatatcagttgtttttattcattttctaaaatgtgctgatcctattaaaaactcctgcttatcttttacaacaaagaaaaatattcaaaaatactgccttcattttcacacacagtgctgaagatgctgcaagcaccaaatcatagctcataaaatcaggtcctgagatagttacccataaagaggaatcctttgagtgtatgccattggtgagccgatgagcatggaccatagaagggctcaatgtagaaggtaaaattggcaaatcataattgagaaatatgactgtgttcccatacataatatggtatagggtgtaatgtacctgctcttgatcactttcattttaaagtgctattcacttaaatgttccatgaattgtttattgcaccacagtttgtgtgtgtgtgtgtgtgtgagagagagagagagagagagagaaataggtcagatcagtatgttgcaagcagatctttagagcctgtcaagttttggttagttgtagtttccatttggaaatgtagatgaatgcttgtagatattggagattgtttctatcttgtaagaacttttcactggtgctgtaagcatttcaaatagcaccagtcttaacctttaaatgggaagtagaaaaggtgagcccaaagtttacagatgattttaatgctatacatgttagtgtagtgatacttagaatgctttgtttgatgtttatttcagaaatgcgtatactagaaaatcattttaatattaaaactggtgacttaatactagttgtaaagtgttttcttaaagaaggatcttggtacttaattgataaagtgggtttagataggagtagcaagtgctctcgatagagaaagtttttgttcacttcaatactttgtcattacaaccagttcttcctgaaaatagttacatgtctagtaaattgatgtagaattaactcgctggagataatctcattttagttctgcaaattctgcctggctttaaaatgcattttcataatacttagaaataatttgactgaaaataactgcttttttatttaatcagtcaatcaacttttactacaaatttaattgagggattttttaatttaattggtgctttaaagaagcaaataaatccctgggttttgttttcttcagtaaatatcctaaagaaactctttaatgtatttgcgagtatatatatattttcttatgcatgctcgatgcattttcgtcctgagaaaagtgttctctacagaaactacccgtgtgttaaaagaagattggcttaaaatggctactgtgatgggaacagtgtcttagggagatgcagcttggacttgaggtaaattgaatactttacaaactggcttagagttttgctttaatgtcattatatgtaaaagggcacatgattattgtaattttgtattctttatggtttccttaattaaaataataaatgtacagtgattact

SEQ ID NO: 266 - DNA, 2552 bp, Mus musculus
  source 1..2552: mol_type = other DNA
gcaagaatgtgatagagcctcgacggccgccatcttcttcctttcttagcagttaaccgagagcggcgcctccctccgcgaggaaacggcagcgtgtgctgtagcgtgggtatggccgactactccacagtgcctccgccgtcgtctggctcggctggcggcggcggcggcggagtcgttaacgacgctttcaaagatgccctgcagagagcgcggcagattgcagcaaaaattgggggtgatgctggtacatcattgaattcaaatgactatggttatgggggacaaaaaagacccttagaagatggagatcagccagatgctaagaaagtacctccccaaaatgactcttttggagcacagttacctccaatgcatcagcagcaaagcagatctgtaatgacagaagaatacaaagtcccagatgggatggtcggatttataattggcagaggaggtgaacagatctcacgaatacagcaggaatctggatgcaagatacagatagcacctgatagtggtggcctaccagaaaggtcttgtatgctaactggaacacctgaatctgtccaatcagcaaaaagattattggaccagattgttgaaaagggaagaccagcccctggctttcatcatggtgatggacctggaaatgcagttcaggaaatcatgattccagccagcaaagcaggactagttattggaaaggggggcgagactattaaacaacttcaggaacgggctggtgttaaaatggtaatgattcaagatgggcctcaaaacactggtgctgataaacctcttaggattacgggtgacccatacaaagttcagcaagccaaggaaatggtattagagttaattcgtgatcaaggtggtttcagagaagtgcggaatgagtatggctcaagaataggaggcaatgaagggatagatgtcccaattccaagatttgctgttggcattgtaataggaagaaatggagaaatgattaaaaaaatacaaaatgatgctggtgttcgaattcagtttaagccagatgatggaacaacacctgataggatagcacagataacaggacctccagacaggtgtcagcacgctgcagaaataatcacagaccttctacgaagtgttcaggctggcaatcctggtggaccgggacctggtggtcgaggacgaggtagaggtcaaggaaactggaatatggggcccccgggtggactccaggagtttaatttcattgtgccaactgggaaaactggactgatcattggaaaaggaggtgaaaccataaaaagcataagccaacagtctggtgcaagaatagaactgcagagaagccctccacctaatgcagatcccaatatgaagttatttacaattcggggcactccacagcaaatagactatgctcgacaactcatagaagagaagattgggggcccagtaaatcctttagggccacctgtaccccatgggccccatggggttccaggtcctcatgggcctcctggacctccagggccgggaactccaatgggaccatacaaccctgcaccttacaatccaggaccacctggcccagctcctcacggtcctccagccccatatgctccccagggatggggaaatgcgtatccacattggcagcaacaggctcctcctgacccagccaaggcaggagcagatccaaactcggcagcttgggctgcttattatgctcactattaccagcagcaggcacaacccccacctgcagctcctgccggtgcaccagctacaacccaaacgaacggacaaggtaactacggagatcagcaggctccagctccagctggacaggttgattatacaaaggcttgggaagaatactacaagaaaatgggtcaagcagttcctgctcctgctggggccccaccaggtggtcagccggattatagtgcagcctgggctgagtactatagacagcaagcagcgtattatgcccagacaagtccccaggggatgccgcagcatcctccagcacctcagggatttgcaaatcatgcaagaagccaccatcatttatattaaccagtttttctttcttaaaggattcactcctgaattagctccatttaaaggattttctttaactttttgtgtatctcttatgtatctcttctgcacagggccaataataagaagtggacaatacagtatttgcttcattgtgtgggggaaaaaacctttgttaaatatatggatgcagacgacttgatgaagatcttaattttgtttttggtttaaagtagtgtttttttccccccctttttttttattttgaaaatgtacaaaataactatcactactgataggaggttaatatttctgtgtagaaatgaaaattggtttgtttttagtctttagtgtagatgtacacattccagcaaatgtatttgcaactattatgtggtcgatgctttgtgatataaatgtactttttcaatgtatactttcacttttaaaatgcctgttttgtgctttacaataaatgatatgaaacctcc

SEQ ID NO: 267 - DNA, 22 bp, Homo sapiens
  source 1..22: mol_type = other DNA
gtgaaaccataaaaagcataag

SEQ ID NO: 268 - DNA, 18 bp, Homo sapiens
  source 1..18: mol_type = other DNA
aaccataaaaagcataag

SEQ ID NO: 269 - DNA, 20 bp, Homo sapiens
  source 1..20: mol_type = other DNA
gtgaaaccataaaaagcata

SEQ ID NO: 270 - DNA, 18 bp, Homo sapiens
  source 1..18: mol_type = other DNA
gtagaaatgaaaattggt

SEQ ID NO: 271 - RNA, 19 nt, synthetic construct
  misc_feature 1..19: note = Synthetic oligonucleotide
  source 1..19: mol_type = other RNA
gacaaacctcttaggatta

SEQ ID NO: 272 - RNA, 19 nt, synthetic construct
  misc_feature 1..19: note = Synthetic oligonucleotide
  source 1..19: mol_type = other RNA
gagaagttcggaatgagta

SEQ ID NO: 273 - RNA, 19 nt, synthetic construct
  misc_feature 1..19: note = Synthetic oligonucleotide
  source 1..19: mol_type = other RNA
gaaaggatagcacaaataa

SEQ ID NO: 274 - RNA, 19 nt, synthetic construct
  misc_feature 1..19: note = Synthetic oligonucleotide
  source 1..19: mol_type = other RNA
aataagaagtggacaatac

SEQ ID NO: 275 - DNA, 18 bp, synthetic construct
  misc_feature 1..18: note = Synthetic oligonucleotide
  source 1..18: mol_type = other DNA
ataaccatagtcatttga

SEQ ID NO: 276 - DNA, 17 bp, synthetic construct
  misc_feature 1..17: note = Synthetic oligonucleotide
  source 1..17: mol_type = other DNA
cccataaccatagtcat

SEQ ID NO: 277 - DNA, 18 bp, synthetic construct
  misc_feature 1..18: note = Synthetic oligonucleotide
  source 1..18: mol_type = other DNA
gagccatctacacataaa

SEQ ID NO: 278 - DNA, 18 bp, synthetic construct
  misc_feature 1..18: note = Synthetic oligonucleotide
  source 1..18: mol_type = other DNA
caagagccatctacacat

SEQ ID NO: 279 - DNA, 18 bp, synthetic construct
  misc_feature 1..18: note = Synthetic oligonucleotide
  source 1..18: mol_type = other DNA
tgtccatttaagaatcca

SEQ ID NO: 280 - DNA, 18 bp, synthetic construct
  misc_feature 1..18: note = Synthetic oligonucleotide
  source 1..18: mol_type = other DNA
ttgtgtccatttaagaat

SEQ ID NO: 281 - DNA, 16 bp, synthetic construct
  misc_feature 1..16: note = Synthetic oligonucleotide
  source 1..16: mol_type = other DNA
tacctttgctgctgat

SEQ ID NO: 282 - DNA, 17 bp, synthetic construct
  misc_feature 1..17: note = Synthetic oligonucleotide
  source 1..17: mol_type = other DNA
gactatacctttgctgc

SEQ ID NO: 283 - DNA, 20 bp, synthetic construct
  misc_feature 1..20: note = Synthetic oligonucleotide
  source 1..20: mol_type = other DNA
actttgtattcttctgtcat

SEQ ID NO: 284 - DNA, 18 bp, synthetic construct
  misc_feature 1..18: note = Synthetic oligonucleotide
  source 1..18: mol_type = other DNA
actttgtattcttctgtc

SEQ ID NO: 285 - DNA, 19 bp, synthetic construct
  misc_feature 1..19: note = Synthetic oligonucleotide
  source 1..19: mol_type = other DNA
gattcaggtgttccagtta

SEQ ID NO: 286 - DNA, 17 bp, synthetic construct
  misc_feature 1..17: note = Synthetic oligonucleotide
  source 1..17: mol_type = other DNA
ttcaaacttactggaca

SEQ ID NO: 287 - DNA, 17 bp, synthetic construct
  misc_feature 1..17: note = Synthetic oligonucleotide
  source 1..17: mol_type = other DNA
tacatctatcccttcat

SEQ ID NO: 288 - DNA, 16 bp, synthetic construct
  misc_feature 1..16: note = Synthetic oligonucleotide
  source 1..16: mol_type = other DNA
ttacatctatcccttc

SEQ ID NO: 289 - DNA, 20 bp, synthetic construct
  misc_feature 1..20: note = Synthetic oligonucleotide
  source 1..20: mol_type = other DNA
cttcctattacaatgccaac

SEQ ID NO: 290 - DNA, 19 bp, synthetic construct
  misc_feature 1..19: note = Synthetic oligonucleotide
  source 1..19: mol_type = other DNA
atttcttcctattacaatg

SEQ ID NO: 291 - DNA, 19 bp, synthetic construct
  misc_feature 1..19: note = Synthetic oligonucleotide
  source 1..19: mol_type = other DNA
ccatttcttcctattacaa

SEQ ID NO: 292 - DNA, 17 bp, synthetic construct
  misc_feature 1..17: note = Synthetic oligonucleotide
  source 1..17: mol_type = other DNA
gtttaaaatacattgcc

SEQ ID NO: 293 - DNA, 19 bp, synthetic construct
  misc_feature 1..19: note = Synthetic oligonucleotide
  source 1..19: mol_type = other DNA
gagtttaaaatacattgcc

SEQ ID NO: 294 - DNA, 18 bp, synthetic construct
  misc_feature 1..18: note = Synthetic oligonucleotide
  source 1..18: mol_type = other DNA
gctttttatggtttcacc

SEQ ID NO: 295 - DNA, 20 bp, synthetic construct
  misc_feature 1..20: note = Synthetic oligonucleotide
  source 1..20: mol_type = other DNA
atgctttttatggtttcacc

SEQ ID NO: 296 - DNA, 19 bp, synthetic construct
  misc_feature 1..19: note = Synthetic oligonucleotide
  source 1..19: mol_type = other DNA
ataatcaacctgtccagct

SEQ ID NO: 297 - DNA, 18 bp, synthetic construct
  misc_feature 1..18: note = Synthetic oligonucleotide
  source 1..18: mol_type = other DNA
ataatcaacctgtccagc

SEQ ID NO: 298 - DNA, 17 bp, synthetic construct
  misc_feature 1..17: note = Synthetic oligonucleotide
  source 1..17: mol_type = other DNA
ataatcaacctgtccag

SEQ ID NO: 299 - DNA, 18 bp, synthetic construct
  misc_feature 1..18: note = Synthetic oligonucleotide
  source 1..18: mol_type = other DNA
tataatcaacctgtccag

SEQ ID NO: 300 - DNA, 19 bp, synthetic construct
  misc_feature 1..19: note = Synthetic oligonucleotide
  source 1..19: mol_type = other DNA
gtataatcaacctgtccag

SEQ ID NO: 301 - DNA, 16 bp, synthetic construct
  misc_feature 1..16: note = Synthetic oligonucleotide
  source 1..16: mol_type = other DNA
ataatcaacctgtcca

SEQ ID NO: 302 - DNA, 17 bp, synthetic construct
  misc_feature 1..17: note = Synthetic oligonucleotide
  source 1..17: mol_type = other DNA
tataatcaacctgtcca

SEQ ID NO: 303 - DNA, 18 bp, synthetic construct
  misc_feature 1..18: note = Synthetic oligonucleotide
  source 1..18: mol_type = other DNA
gtataatcaacctgtcca

SEQ ID NO: 304 - DNA, 17 bp, synthetic construct
  misc_feature 1..17: note = Synthetic oligonucleotide
  source 1..17: mol_type = other DNA
gtataatcaacctgtcc

SEQ ID NO: 305 - DNA, 16 bp, synthetic construct
  misc_feature 1..16: note = Synthetic oligonucleotide
  source 1..16: mol_type = other DNA
tataatcaacctgtcc

SEQ ID NO: 306 - DNA, 17 bp, synthetic construct
  misc_feature 1..17: note = Synthetic oligonucleotide
  source 1..17: mol_type = other DNA
cccattttcttgtagta

SEQ ID NO: 307 - DNA, 18 bp, synthetic construct
  misc_feature 1..18: note = Synthetic oligonucleotide
  source 1..18: mol_type = other DNA
acccattttcttgtagta

SEQ ID NO: 308 - DNA, 19 bp, synthetic construct
  misc_feature 1..19: note = Synthetic oligonucleotide
  source 1..19: mol_type = other DNA
tacccattttcttgtagta

SEQ ID NO: 309 - DNA, 20 bp, synthetic construct
  misc_feature 1..20: note = Synthetic oligonucleotide
  source 1..20: mol_type = other DNA
atacccattttcttgtagta

SEQ ID NO: 310 - DNA, 18 bp, synthetic construct
  misc_feature 1..18: note = Synthetic oligonucleotide
  source 1..18: mol_type = other DNA
atacccattttcttgtag

SEQ ID NO: 311 - DNA, 19 bp, synthetic construct
  misc_feature 1..19: note = Synthetic oligonucleotide
  source 1..19: mol_type = other DNA
catacccattttcttgtag

SEQ ID NO: 312 - DNA, 18 bp, synthetic construct
  misc_feature 1..18: note = Synthetic oligonucleotide
  source 1..18: mol_type = other DNA
catacccattttcttgta

SEQ ID NO: 313 - DNA, 18 bp, synthetic construct
  misc_feature 1..18: note = Synthetic oligonucleotide
  source 1..18: mol_type = other DNA
tggagctaattcaggagt

SEQ ID NO: 314 - DNA, 20 bp, synthetic construct
  misc_feature 1..20: note = Synthetic oligonucleotide
  source 1..20: mol_type = other DNA
aaatggagctaattcaggag

SEQ ID NO: 315 - DNA, 19 bp, synthetic construct
  misc_feature 1..19: note = Synthetic oligonucleotide
  source 1..19: mol_type = other DNA
ttgtccacttcttattatt

SEQ ID NO: 316 - DNA, 18 bp, synthetic construct
  misc_feature 1..18: note = Synthetic oligonucleotide
  source 1..18: mol_type = other DNA
ccccacacaatgaagcaa

SEQ ID NO: 317 - DNA, 19 bp, synthetic construct
  misc_feature 1..19: note = Synthetic oligonucleotide
  source 1..19: mol_type = other DNA
ttcatcaagtcgtctgcat

SEQ ID NO: 318 - DNA, 18 bp, synthetic construct
  misc_feature 1..18: note = Synthetic oligonucleotide
  source 1..18: mol_type = other DNA
gatcttcatcaagtcgtc

SEQ ID NO: 319 - DNA, 19 bp, synthetic construct
  misc_feature 1..19: note = Synthetic oligonucleotide
  source 1..19: mol_type = other DNA
atattaacctcctatcagt

SEQ ID NO: 320 - DNA, 19 bp, synthetic construct
  misc_feature 1..19: note = Synthetic oligonucleotide
  source 1..19: mol_type = other DNA
aatattaacctcctatcag

SEQ ID NO: 321 - DNA, 19 bp, synthetic construct
  misc_feature 1..19: note = Synthetic oligonucleotide
  source 1..19: mol_type = other DNA
atttatatcacaaagcatc

SEQ ID NO: 322 - DNA, 18 bp, synthetic construct
  misc_feature 1..18: note = Synthetic oligonucleotide
  source 1..18: mol_type = other DNA
aagtacatttatatcaca

SEQ ID NO: 323 - DNA, 20 bp, synthetic construct
  misc_feature 1..20: note = Synthetic oligonucleotide
  source 1..20: mol_type = other DNA
catttattgtaaagcacaaa

SEQ ID NO: 324 - DNA, 18 bp, synthetic construct
  misc_feature 1..18: note = Synthetic oligonucleotide
  source 1..18: mol_type = other DNA
atcatttattgtaaagca

SEQ ID NO: 325 - DNA, 17 bp, synthetic construct
  misc_feature 1..17: note = Synthetic oligonucleotide
  source 1..17: mol_type = other DNA
cttatgctttttatggt

SEQ ID NO: 326 - DNA, 18 bp, synthetic construct
  misc_feature 1..18: note = Synthetic oligonucleotide
  source 1..18: mol_type = other DNA
cttatgctttttatggtt

SEQ ID NO: 327 - DNA, 17 bp, synthetic construct
  misc_feature 1..17: note = Synthetic oligonucleotide
  source 1..17: mol_type = other DNA
gctttttatggtttcac

SEQ ID NO: 328 - DNA, 18 bp, synthetic construct
  misc_feature 1..18: note = Synthetic oligonucleotide
  source 1..18: mol_type = other DNA
tatgctttttatggtttc

SEQ ID NO: 329 - DNA, 18 bp, synthetic construct
  misc_feature 1..18: note = Synthetic oligonucleotide
  source 1..18: mol_type = other DNA
accaattttcatttctac

SEQ ID NO: 330 - DNA, 16 bp, synthetic construct
  misc_feature 1..16: note = Synthetic oligonucleotide
  source 1..16: mol_type = other DNA
ccccataaccatagtc

SEQ ID NO: 331 - DNA, 21 bp, synthetic construct
  misc_feature 1..21: note = Synthetic oligonucleotide
  source 1..21: mol_type = other DNA
ccatcctggacattgaggact

SEQ ID NO: 332 - DNA, 21 bp, synthetic construct
  misc_feature 1..21: note = Synthetic oligonucleotide
  source 1..21: mol_type = other DNA
caggttccgggacaggtagta

SEQ ID NO: 333 - DNA, 21 bp, synthetic construct
  misc_feature 1..21: note = Synthetic oligonucleotide
  source 1..21: mol_type = other DNA
cgtctgtgccttctcatctgc

SEQ ID NO: 334 - DNA, 20 bp, synthetic construct
  misc_feature 1..20: note = Synthetic oligonucleotide
  source 1..20: mol_type = other DNA
gcacagcttggaggcttgaa

SEQ ID NO: 335 - DNA, 20 bp, synthetic construct
  misc_feature 1..20: note = Synthetic oligonucleotide
  source 1..20: mol_type = other DNA
ccgtctgaactatcctgccc

SEQ ID NO: 336 - DNA, 20 bp, synthetic construct
  misc_feature 1..20: note = Synthetic oligonucleotide
  source 1..20: mol_type = other DNA
gccgtagtcggtgtactcgt

SEQ ID NO: 337 - DNA, 18 bp, synthetic construct
  misc_feature 1..18: note = Synthetic oligonucleotide
  source 1..18: mol_type = other DNA
atgctttttatggtttca

SEQ ID NO: 338 - DNA, 18 bp, synthetic construct
  misc_feature 1..18: note = Synthetic oligonucleotide
  source 1..18: mol_type = other DNA
ttatgctttttatggttt

SEQ ID NO: 339 - DNA, 24 bp, synthetic construct
  misc_feature 1..24: note = Synthetic oligonucleotide
  source 1..24: mol_type = other DNA
gtttttcacctctgcctaatcatc

SEQ ID NO: 340 - DNA, 23 bp, synthetic construct
  misc_feature 1..23: note = Synthetic oligonucleotide
  source 1..23: mol_type = other DNA
gcaaaaagttgcatggtgctggt

SEQ ID NO: 341 - DNA, 19 bp, synthetic construct
  misc_feature 1..19: note = Synthetic oligonucleotide
  source 1..19: mol_type = other DNA
ccgtgtgcacttcgcttca

SEQ ID NO: 342 - DNA, 19 bp, synthetic construct
  misc_feature 1..19: note = Synthetic oligonucleotide
  source 1..19: mol_type = other DNA
gcacagcttggaggcttga

SEQ ID NO: 343 - DNA, 23 bp, synthetic construct
  misc_feature 1..23: note = Synthetic oligonucleotide
  source 1..23: mol_type = other DNA
catggagaccaccgtgaacgccc

SEQ ID NO: 344 - DNA, 20 bp, synthetic construct
  misc_feature 1..20: note = Synthetic oligonucleotide
  source 1..20: mol_type = other DNA
gaggcaggtcccctagaaga

SEQ ID NO: 345 - DNA, 21 bp, synthetic construct
  misc_feature 1..21: note = Synthetic oligonucleotide
  source 1..21: mol_type = other DNA
ggagtgtggattcgcactcct

SEQ ID NO: 346 - DNA, 20 bp, synthetic construct
  misc_feature 1..20: note = Synthetic oligonucleotide
  source 1..20: mol_type = other DNA
agattgagatcttctgcgac

SEQ ID NO: 347 - DNA, 16 bp, Homo sapiens
  source 1..16: mol_type = other DNA
gactatggttatgggg

SEQ ID NO: 348 - DNA, 39 bp, synthetic construct
  source 1..39: mol_type = other DNA
ccccataaccatagtccactttattataacttgaatctc

SEQ ID NO: 349 - DNA, 39 bp, synthetic construct
  source 1..39: mol_type = other DNA
ctttattataacttgaatctccaccccataaccatagtc

SEQ ID NO: 350 - DNA, 38 bp, synthetic construct
  source 1..38: mol_type = other DNA
cttatgctttttatggttcattacatactctggtcaaa

SEQ ID NO: 351 - DNA, 38 bp, synthetic construct
  source 1..38: mol_type = other DNA
ttacatactctggtcaaacacttatgctttttatggtt

SEQ ID NO: 352 - DNA, 20 bp, synthetic construct
  source 1..20: mol_type = other DNA
ccaaatcttataataactac

SEQ ID NO: 353 - DNA, 19 bp, synthetic construct
  source 1..19: mol_type = other DNA
ctgtgccttgggtggcttt

SEQ ID NO: 354 - DNA, 24 bp, synthetic construct
  source 1..24: mol_type = other DNA
aaggaaagaagtcagaaggcaaaa

SEQ ID NO: 355 - DNA, 34 bp, synthetic construct
  source 1..34: mol_type = other DNA
  misc_feature 9..10: note = ZEN between nucleotides 9 and 10
agctccaaattctttataagggtcgatgtccatg

(19) United States
(12) Patent Application Publication
(10) Pub. No.: US 2024/0409939 A1
(43) Pub. Date: Dec. 12, 2024

(54) COMBINATION OF OLIGONUCLEOTIDES FOR MODULATING RTEL1 AND FUBP1

(71) Applicant: Hoffmann-La Roche Inc., Little Falls, NJ (US)

(72) Inventors: Konrad BLEICHER, Freiburg (DE); Josephine Felber, Riehen (CH); Erik Funder, Hillerød (DK); Souphalone Luangsay, Basel (CH); Tobias Nilsson, Basel (CH); Soren Ottosen, Glostrup (DK); Johanna Marie Pose Vicente, Birsfelden (CH); Timothy Tellinghuisen, Oberwil (CH)

(21) Appl. No.: 18/742,763

(22) Filed: Jun. 13, 2024

Related U.S. Application Data
(63) Continuation of application No. PCT/EP2022/086212, filed on Dec. 15, 2022.

(30) Foreign Application Priority Data
Dec. 17, 2021 (EP) 21215511.3

(51) Int. Cl. C12N 15/113; A61P 31/20

(52) U.S. Cl. CPC C12N 15/113; A61P 31/20; C12N 2310/113; C12N 2310/315; C12N 2310/3231; C12N 2310/3341; C12N 2310/3519

(57) ABSTRACT

The present invention relates to combinations of Regulator of telomere elongation helicase 1 (RTEL1) and Far Upstream Element-Binding Protein 1 (FUBP1) inhibitors, such as oligonucleotides (oligomers) that are complementary to RTEL1 or FUBP1, respectively, leading to modulation of the expression of RTEL1 and FUBP1 or modulation of RTEL1 and FUBP1 activity. The invention in particular relates to a combination of an inhibitor of RTEL1 and an inhibitor of FUBP1 for use in treating and/or preventing a disease, preferably a hepatitis B virus (HBV) infection, in particular a chronic HBV infection. The invention in particular relates to the use of a combination of RTEL1 and FUBP1 inhibitors for destabilizing cccDNA, such as HBV cccDNA. Also comprised in the present invention is a pharmaceutical composition, a kit and the use thereof in the treatment and/or prevention of a HBV infection.

### SEQUENCE LISTING

The instant application contains a Sequence Listing which has been submitted electronically in XML format and is hereby incorporated by reference in its entirety. Said XML copy, created on Jun. 10, 2024, is named 51551-021001_Sequence_Listing_6_10_24.xml and is 665,833 bytes in size.

### FIELD OF INVENTION

The present invention relates to combinations of Regulator of telomere elongation helicase 1 (RTEL1) and Far Upstream Element-Binding Protein 1 (FUBP1) inhibitors, such as oligonucleotides (oligomers) that are complementary to RTEL1 or FUBP1, respectively, leading to modulation of the expression of RTEL1 and FUBP1 or modulation of RTEL1 and FUBP1 activity. The invention in particular relates to a combination of an inhibitor of RTEL1 and an inhibitor of FUBP1 for use in treating and/or preventing a disease, preferably a hepatitis B virus (HBV) infection, in particular a chronic HBV infection. The invention in particular relates to the use of a combination of RTEL1 and FUBP1 inhibitors for destabilizing cccDNA, such as HBV cccDNA. Also comprised in the present invention is a pharmaceutical composition, a kit and the use thereof in the treatment and/or prevention of a HBV infection.

### BACKGROUND

Hepatitis B is an infectious disease caused by the hepatitis B virus (HBV), a small hepatotropic virus that replicates through reverse transcription. Chronic HBV infection is a key factor for severe liver diseases such as liver cirrhosis and hepatocellular carcinoma. Current treatments for chronic HBV infection are based on administration of pegylated type 1 interferons or nucleos(t)ide analogues, such as lamivudine, adefovir, entecavir, tenofovir disoproxil, and tenofovir alafenamide, which target the viral polymerase, a multifunctional reverse transcriptase. Treatment success is usually measured as loss of hepatitis B surface antigen (HBsAg). However, a complete HBsAg clearance is rarely achieved since Hepatitis B virus DNA persists in the body after infection. HBV persistence is mediated by an episomal form of the HBV genome which is stably maintained in the nucleus. This episomal form is called “covalently closed circular DNA” (cccDNA). The cccDNA serves as a template for all HBV transcripts, including pregenomic RNA (pgRNA), a viral replicative intermediate. The presence of a few copies of cccDNA might be sufficient to reinitiate a full-blown HBV infection. Current treatments for HBV do not target cccDNA. A cure of chronic HBV infection, however, would require the elimination of cccDNA (reviewed by Nassal, Gut. 2015 December; 64 (12): 1972-84. doi: 10.1136/gutjnl-2015-309809).

Regulator of telomere elongation helicase 1 (RTEL1) encodes a DNA helicase which functions in the stability, protection and elongation of telomeres and interacts with proteins in the shelterin complex known to protect telomeres during DNA replication. Mutations in this gene have been associated with dyskeratosis congenita and Hoyerall-Hreidarsson syndrome (See for example review by Vannier et al 2014 Trends Cell Biol. Vol 24 p. 416).

Located in the nucleus, RTEL1 functions as an ATP-dependent DNA helicase implicated in telomere-length regulation, DNA repair and the maintenance of genomic stability. RTEL1 Acts as an anti-recombinase to counteract toxic recombination and limit crossover during meiosis and regulates meiotic recombination and crossover homeostasis by physically dissociating strand invasion events and thereby promotes non-crossover repair by meiotic synthesis dependent strand annealing (SDSA) as well as disassembly of D loop recombination intermediates. In additional RTEL1 disassembles T loops and prevents telomere fragility by counteracting telomeric G4-DNA structures, which together ensure the dynamics and stability of the telomere.

RTEL1 has been identified in a siRNA screen as a stabilizer of HPV episomes: (Edwards et al 2013 PLOS One Vol 8, e75406). siRNA targeting RTEL1 has likewise been used to identify interactants with RTEL1 in Hoyeraal-Hreidarsson syndrome (Schertzer et al 2015 Nucleic Acid Res Vol 43 p. 1834). In addition, RTEL1 was identified as a HIV host dependency factor from a siRNA screen for essential host proteins to provide targets for inhibition HIV infection (WO 2007/094818).

WO2020011902A1 relates to a RTEL1 inhibitor for use in treatment of an HBV infection, in particular a chronic HBV infection.

Far Upstream Element-Binding Protein 1 (FUBP1 or FBP1) is a single stranded DNA-binding protein that binds to multiple DNA elements. This protein is also thought to bind RNA and contains 3′-5′ helicase activity with in vitro activity on both DNA-DNA and RNA-RNA duplexes. FUBP1 is known to activate the transcription of the proto-oncogene c-myc by binding to far upstream element (FUSE) located upstream of c-myc in undifferentiated cells. The protein is primarily present in the nucleus of the cell. Upregulation of FUBP1 has been observed in many types of cancers. Furthermore, FUBP1 can bind to and mediate replication of RNA from Hepatitis C virus and Enterovirus (Zhang and Chen 2013 Oncogene vol 32 p. 2907-2916).

FUBP1 has also been identified in Hepatocellular carcinoma (HCC) where it has been suggested to be involved in HCC tumorigenesis (Ramdzan et al 2008 Proteomics Vol 8 p. 5086-5096) and that FUBP1 is required for HCC tumour growth as illustrated using lentivirus expressed shRNA targeting FUBP1 (Rabenhorst et al 2009 Hepatology vol 50 p 1121-1129).

It has been demonstrated that knock down of FUBP1 with lentivirus expressed shRNA's enhances treatment response in ovarian cancer (Zhang et al 2017 Oncology Letters Vol 14 p. 5819-5824).

WO 2004/027061 disclose a screening method which involves the step of analyzing whether or not a test substance inhibits FBP and a medicinal composition for treating a proliferative disease which contains as the active ingredient(s) a substance inhibiting FBP.

Some small molecules inhibiting FUBP1 have been identified, all with the purpose of treating cancer (Huth et al 2004 J Med. Chen Vol 47 p. 4851-4857; Hauck et al 2016 Bioorganic & Medicinal Chemistry Vol 24 p. 5717-5729 Hosseini et al 2017 Biochemical Pharmacology Vol 146 p. 53-62 and Xiong et al 2016 Int J Onc vol 49 p 623). WO2004/017940 describes lipid based formulations of SN-38, it claims treatment of viral infection, in particular HIV, there is however no example supporting this.

Poly (U) Binding Splicing Factor 60 (PUF60) is a potentially regulator of both transcriptional and post-transcriptional steps of HBV pregenome expression. PUF60 is known to form a complex with FUBP1 in relation to c-myc repression. FUBP1 does, however, not participate in the PUF60 dependent regulation of HBV pregenome expression (Sun et al 2017 Scientific Reports 7:12874).

HBV infection remains a major health problem worldwide, which concerns an estimated 350 million chronic carriers. Approximately 25% of carriers die from chronic hepatitis, cirrhosis, or liver cancer. Hepatitis B virus is the second most significant carcinogen behind tobacco, causing from 60% to 80% of all primary liver cancer. HBV is 100 times more contagious than HIV.

WO2019/193165A1 relates to FUBP1 inhibitors for use in treatment of an HBV infection.

### Objective of the Invention

### SUMMARY OF THE INVENTION

The present invention relates to a combination of an inhibitor of RTEL1 and an inhibitor of FUBP1, such as a composition or a pharmaceutical composition comprising an inhibitor of RTEL1 and an inhibitor of FUBP1. The inhibitor of RTEL1 is capable of inhibiting the expression and/or activity of RTEL1; and the inhibitor of FUBP1 is capable of inhibiting the expression and/or activity of FUBP1. Suitably, the inhibitor of RTEL1 is capable of inhibiting the expression of a RTEL1 nucleic acid. Suitably, the inhibitor of FUBP1 is capable of inhibiting the expression of a FUBP1 nucleic acid. The invention further relates to said combination, composition or pharmaceutical composition for use in the treatment or prevention of a disease.

The invention also relates to a kit comprising an inhibitor of RTEL1 and an inhibitor of FUBP1. The inhibitor of RTEL1 is capable of inhibiting the expression and/or activity of RTEL1; and the inhibitor of FUBP1 is capable of inhibiting the expression and/or activity of FUBP1. Suitably, the inhibitor of RTEL1 is capable of inhibiting the expression of a RTEL1 nucleic acid. Suitably, the inhibitor of FUBP1 is capable of inhibiting the expression of a FUBP1 nucleic acid. The invention further relates to said kit for use in the treatment or prevention of a disease.

The invention also relates to a method for treating or preventing a disease comprising administering a therapeutically or prophylactically effective amount of an inhibitor of RTEL1, to a subject suffering from or susceptible to the disease, wherein the method further comprises the administration of an effective amount of an inhibitor of FUBP1.

The invention also relates to a method for treating or preventing a disease comprising administering a therapeutically or prophylactically effective amount of an inhibitor of FUBP1, to a subject suffering from or susceptible to the disease, wherein the method further comprises the administration of an effective amount of an inhibitor of RTEL1.

The invention also relates to a method for treating or preventing a disease comprising administering a combination of a therapeutically or prophylactically effective amount of an inhibitor of RTEL1 and a therapeutically or prophylactically effective amount of an inhibitor of FUBP1 to a subject suffering from or susceptible to the disease.

The invention also relates to the use of an inhibitor of FUBP1 and an inhibitor of RTEL1, for the preparation of a medicament for treatment or prevention of hepatitis B virus (HBV) and/or cancer.

The invention also relates to an in vivo or in vitro method for modulating RTEL1 and FUBP1 expression in a target cell which is expressing RTEL1 and FUBP1, said method comprising administering an inhibitor of RTEL1 and an inhibitor of FUBP1; in an effective amount to said cell.

In a particular embodiment, the disease is a hepatitis B virus (HBV) infection and/or cancer.

In a particular embodiment, the disease is a chronic hepatitis B virus (HBV) infection.

The present inventors have surprisingly demonstrated that combinations of RTEL1 and FUBP1 inhibitors provide a synergistic inhibition of HBV.

### SEQUENCE LISTING

The sequence listing submitted with this application is hereby incorporated by reference. In the event of a discrepancy between the sequence listing and the specification or figures, the information disclosed in the specification (including the figures) shall be deemed to be correct.

### BRIEF DESCRIPTION OF FIGURES

FIG. 1 Compound 243_1 (SEQ ID NO: 243) conjugated to a trivalent GalNAc moiety via a phosphodiester linked DNA dinucleotide

FIG. 1A Residue A of Compound 243_1 (SEQ ID NO: 243)

FIG. 2 Compound 244_1 (SEQ ID NO: 244) conjugated to a trivalent GalNAc moiety via a phosphodiester linked DNA dinucleotide

FIG. 2A Residue A of Compound 244_1 (SEQ ID NO: 244)

FIG. 3 Compound 245_1 (SEQ ID NO: 245) conjugated to a trivalent GalNAc moiety via a phosphodiester linked DNA dinucleotide

FIG. 3A Residue A of Compound 245_1 (SEQ ID NO: 245)

FIG. 4 Compound 246_1 (SEQ ID NO: 246) conjugated to a trivalent GalNAc moiety via a phosphodiester linked DNA dinucleotide

FIG. 4A Residue A of Compound 246_1 (SEQ ID NO: 246)

FIG. 5 FIG. 5 illustrates exemplary GalNAc moieties. The compound in FIG. 5L is composed of monomeric GalNAc phosphoramidites added to the oligonucleotide while still on the solid support as part of the synthesis, X is S or O, Y is Sor O, and n=1-3 (see WO 2017/178656). FIG. 5B and FIG. 5D are also termed GalNAc2 or GN2 herein, without and with C6 linker, respectively.

FIG. 6 FIG. 6A-L: Illustrates exemplary antisense oligonucleotide conjugates, wherein the oligonucleotide is represented by the term “A” as described above. Compounds in FIG. 6A-D comprise a di-lysine brancher molecule, a PEG3 spacer and three terminal GalNAc carbohydrate moieties. In the compounds in FIG. 6A (FIG. 6A-1 and FIG. 6A-2 show two different diastereoisomers of the same compound) and FIG. 6B (FIG. 6B-1 and FIG. 6B-2 show two different diastereoisomers of the same compound) the oligonucleotide is attached directly to the asialoglycoprotein receptor targeting conjugate moiety without an alkyl linker. In the compounds in FIG. 6C (FIG. 6C-1 and FIG. 6C-2 show two different diastereoisomers of the same compound) and FIG. 6D (FIG. 6D-1 and FIG. 6D-2 show two different diastereoisomers of the same compound) the oligonucleotide is attached to the asialoglycoprotein receptor targeting conjugate moiety via a C6 linker. The compounds in FIG. 6E-K comprise a commercially available trebler brancher molecule and spacers of varying length and structure and three terminal GalNAc carbohydrate moieties. The compound in FIG. 6L is composed of monomeric GalNAc phosphoramidites added to the oligonucleotide while still on the solid support as part of the synthesis, wherein X═S or O, and independently Y═S or O, and n=1-3 (see WO 2017/178656).

FIG. 7: Testing oligonucleotide CMP ID Nos 243_1, 244_1, 245_1 and 246_1 in vitro for concentration dependent potency and efficacy in human cell line MDA-MB-231.

FIG. 8 Compound 325_1 (SEQ ID NO: 325) conjugated to a GalNAc moiety via a phosphodiester linked DNA dinucleotide

FIG. 8A Residue A of Compound 325_1 (SEQ ID NO: 325)

FIG. 9 Compound 325_2 (SEQ ID NO: 325) conjugated to a GalNAc moiety via a phosphodiester linked DNA dinucleotide

FIG. 9A Residue A of Compound 325_2 (SEQ ID NO: 325)

FIG. 10 Compound 326_1 (SEQ ID NO: 326) conjugated to a GalNAc moiety via a phosphodiester linked DNA dinucleotide

FIG. 10A Residue A of Compound 326_1 (SEQ ID NO: 326)

FIG. 11 Compound 326_2 (SEQ ID NO: 326) conjugated to a GalNAc moiety via a phosphodiester linked DNA dinucleotide FIG. 11A Residue A of Compound 326_2 (SEQ ID NO: 326)

FIG. 12 Compound 326_3 (SEQ ID NO: 326) conjugated to a GalNAc moiety via a phosphodiester linked DNA dinucleotide

FIG. 12A Residue of Compound 326_3 (SEQ ID NO: 326)

FIG. 13 Compound 326_4 (SEQ ID NO: 326) conjugated to a GalNAc moiety via a phosphodiester linked DNA dinucleotide

FIG. 13A Residue A of Compound 326_4 (SEQ ID NO: 326)

FIG. 14 Compound 327_1 (SEQ ID NO: 327) conjugated to a GalNAc moiety via a phosphodiester linked DNA dinucleotide

FIG. 14A Residue A of Compound 327_1 (SEQ ID NO: 327)

FIG. 15 Compound 328_1 (SEQ ID NO: 328) conjugated to a GalNAc moiety via a phosphodiester linked DNA dinucleotide

FIG. 15A Residue A of Compound 328_1 (SEQ ID NO: 328)

FIG. 16 Compound 329_1 (SEQ ID NO: 329) conjugated to a GalNAc moiety via a phosphodiester linked DNA dinucleotide

FIG. 16A Residue A of Compound 329_1 (SEQ ID NO: 329)

FIG. 17 illustrates the results of an analysis of the in vitro efficacy of anti-FUBP1 compounds in Hela cells. FUBP1 mRNA levels are normalized and shown as % of control.

FIG. 18 Target engagement: FUBP1 mRNA. As described in Example 2.3, four antisense oligonucleotide compounds have been tested in HBV infected PHH cells. Each compound has been delivered to cells at a concentration of 10 μM once per week for three weeks. FUBP1 mRNA target KD has been evaluated one week after the last treatment. Total RNA has been extracted from cells using a MagNA Pure robot and the MagNA Pure 96 Cellular RNA Large Volume Kit according to the manufacturer's protocol and FUBP1 mRNA quantified by TaqMan qPCR. The figure shows the residual expression of the Target mRNA compared to negative control (NDC=1) with oligos tested at 10 μM. Data are normalized to the human GUS B reference gene and the mean+SD from two biological replicates are reported for each oligo tested. FC of 50% and 20% are highlighted on the graph. CMP ID NO: 326_3 shows the best FUBP1 mRNA KD with 80% reduction mRNA expression respectively at 10 μM. CMP ID NO: 329_1 shows the strongest effect in reducing FUBP1 mRNA compared to the prior art oligos (CMP ID Nos: 276_1 and 291_1), equally to the oligonucleotide with CMP ID NO: 326_3. They both reduce target mRNA expression at 10 μM by about 80% compared to the NDC.

FIG. 19 Southern blot of intrahepatic HBV DNA revealing a reduction of the cccDNA and total HBV DNA in the FUBP1 and RTEL1 LNA mono-treatment arms which is further enhanced in the FUBP1+RTEL1 combination arm. Southern blot of total DNA extracts from PXB mouse livers using a HBV specific full genome length probe for detection. DNA concentrations were adjusted by NanoDrop and 15 ug DNA loaded per lane. The red box indicates the cccDNA bands.

FIG. 20 Semi-quantification of intrahepatic cccDNA and total HBV DNA levels by qPCR.

FIG. 21 Kinetic of baseline corrected serum HBV DNA levels.

FIG. 22 Kinetic of baseline corrected serum HBsAg

FIG. 23 Kinetic of baseline corrected serum HBeAg

FIG. 24 Intrahepatic target engagement and efficacy of RTEL1 and FUBP1 LNA molecules assessed by RT-qPCR

FIG. 25 In vitro reduction of intrahepatic HBV pRNA in HBV infected PHH using single FUBP1 ASO (GalNAc-326_3), single RTEL1 ASO (GalNAc-245_1), two RTEL1/FUBP1 dual ASOs (Gal-NAc-350_1 and Gal-NAc-351_1), a combination of FUBP1 ASO (GalNAc-326_3)+RTEL1 ASO (GalNAc-245_1) and a negative control (Ga-NAc-352_1) for reference.

FIG. 26 In vitro reduction of intrahepatic HBV RNA in HBV infected PHH using single FUBP1 ASO (GalNAc-326_3), single RTEL1 ASO (GalNAc-245_1), two RTEL1/FUBP1 dual ASOs (Gal-NAc-350_1 and Gal-NAc-351_1), a combination of FUBP1 ASO (GalNAc-326_3)+RTEL1 ASO (GalNAc-245_1) and a negative control (Ga-NAc-352_1) for reference.

FIG. 27 Dose-response curves of RTEL1 Gene Expression and associated EC50 values of conjugated versions of CMP IDs NO 352_1 (control), 326_3 (FUBP1), 245_1 (RTEL1), 350_1 (Dual), 351_1 (Dual) and 326_3 (FUBP1)+245_1 (RTEL1) administered separately (i.e. added as two individual ASOs).

FIG. 28 Dose-response curves of FUBP1 Gene Expression and associated EC50 values of conjugated versions of CMP IDs NO 352_1 (control), 326_3 (FUBP1), 245_1 (RTEL1), 350_1 (Dual), 351_1 (Dual) and 326_3 (FUBP1)+245_1 (RTEL1) administered separately (i.e. added as two individual ASOs).

### DEFINITIONS

### 2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3 (2), 293-213, and Deleavey and Damha, Chemistry and Biology 2012, 19, 937. Below are illustrations of some 2′ substituted modified nucleosides.

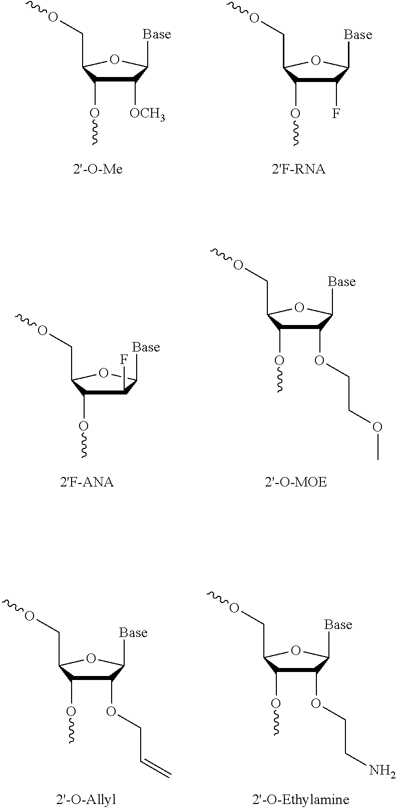

In relation to the present invention 2′ substituted sugar modified nucleosides does not include 2′ bridged nucleosides like LNA.

### Alternating Flank Gapmers

Flanking regions may comprise both LNA and DNA nucleoside and are referred to as “alternating flanks” as they comprise an alternating motif of LNA-DNA-LNA nucleosides. Gapmers comprising at least one alternating flank are referred to as “alternating flank gapmers”. “Alternative flank gapmers” are thus LNA gapmer oligonucleotides where at least one of the flanks (F or F′) comprises DNA in addition to the LNA nucleoside(s). In some embodiments, at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides. Alternating flank LNA gapmers are disclosed in WO2016/127002.

An alternating flank region may comprise up to 3 contiguous DNA nucleosides, such as 1 to 2 or 1 or 2 or 3 contiguous DNA nucleosides.

The alternating flak regions can be annotated as a series of integers, representing a number of LNA nucleosides (L) followed by a number of DNA nucleosides (D), for example [L]1-3-[D]1-3-[L]1-3 or [L]1-2-[D]1-2-[L]1-2-[D]1-2-[L]1-2. In oligonucleotide designs these will often be represented as numbers such that 2-2-1 represents 5′ [L]2-[D]2-[L]3′, and 1-1-1-1-1 represents 5′ [L]-[D]-[L]-[D]-[L]3′. The length of the flank (region F and F′) in oligonucleotides with alternating flanks may be as described herein above for these regions, such as 4 to 8, such as 5 to 6 nucleosides, such as 4, 5, 6 or 7 modified nucleosides. It may be advantageous to have at least two LNA nucleosides at the 3′ end of the 3′ flank (F′), to confer additional exonuclease resistance.

In an embodiment, a gapmer oligonucleotide for use in the present invention can be represented by the following formula:

F₄₋₆-G₇₋₁₁-F′₂₋₆,

wherein F is has a design of [L]₁₋₃-[D]₁₋₃-[L]₁₋₃ and F′ has a design of [L]₁₋₂-[D]₁₋₂-[L]₂₋₄, or [L]₂₋₆
with the proviso that the overall length of the gapmer regions F-G-F′ is at least 16 nucleotides, such as 17 or 18 nucleotides in length.

Thus, the gapmer oligonucleotide of the present invention may comprise at least one alternating flank. Typically, at least the F region is an alternating flank. In some embodiments, the both the F and the F′ regions are alternating flanks. In some embodiments, the F region is an alternating flank and the F′ region is a uniform flank (i.e. F′ consists of only one type of sugar modified nucleosides, such as only beta-D-oxy LNA).

In some embodiments, the design of region F is selected from a design of 3-2-1 (i.e. LLLDDL), 3-1-1 (i.e. LLLDL), 2-1-2 (LLDLL), 2-1-1 (LLDL) and 1-3-1 (i.e. LDDDL).

In some embodiments, the design of region F′ is 1-1-3 (i.e. LDLLL) or 1-1-2 (i.e. LDLL). In some embodiments, the design of region F is LL, LLL or LLLL.

### Antisense Oligonucleotides

The term “antisense oligonucleotide”, or “ASO”, as used herein is defined as oligonucleotides capable of modulating expression of a target gene by hybridizing to a target nucleic acid, in particular to a contiguous sequence on a target nucleic acid. The antisense oligonucleotides herein are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide.

Advantageously, the single stranded antisense oligonucleotide does not contain RNA nucleosides, since this will decrease nuclease resistance.

Advantageously, the oligonucleotide of the combination of the invention comprises one or more modified nucleosides or nucleotides, such as 2′ sugar modified nucleosides. Furthermore, it is advantageous that the nucleosides which are not modified are DNA nucleosides.

cccDNA (Covalently Closed Circular DNA)

cccDNA (covalently closed circular DNA) is a special DNA structure that arises during the propagation of some DNA viruses (Polyomaviridae) in the cell nucleus. cccDNA is a double-stranded DNA that originates in a linear form that is ligated by means of DNA ligase to a covalently closed ring. In most cases, transcription of viral DNA can occur from the circular form only. The cccDNA of viruses is also known as episomal DNA or occasionally as a minichromosome.

cccDNA is typical of Caulimoviridae and Hepadnaviridae, including the hepatitis B virus (HBV). The HBV genome forms a stable minichromosome, the covalently closed circular DNA (cccDNA), in the hepatocyte nucleus. The cccDNA is formed by conversion of capsid-associated relaxed circular DNA (rcDNA). HBV cccDNA formation involves a multi-step process that requires the cellular DNA repair machinery and relies on specific interactions with distinct cellular components that contribute to the completion of the positive strand DNA in rcDNA (Alweiss et al. 2017, Viruses, 9 (6): 156).

cccDNA is the viral genetic template that resides in the nucleus of infected hepatocytes, where it gives rise to all HBV RNA transcripts needed for productive infection and is responsible for viral persistence during natural course of chronic HBV infection (Locarnini & Zoulim, 2010 Antivir Ther. 15 Suppl 3:3-14. doi: 10.3851/IMP1619). Acting as a viral reservoir, cccDNA is the source of viral rebound after cessation of treatment, necessitating long term, often, lifetime treatment. PEG-IFN can only be administered to a small subset of CHB due to its various side effects.

Consequently, novel therapies that can deliver a complete cure, defined by degradation or elimination of HBV cccDNA, to the majority of CHB patients are highly needed.

### Combination

The term “combination” is understood as the combination at least two different active compounds or prodrugs (medical compounds or medicaments) for treatment of a disease. A pharmaceutical combination can involve compounds that are physically, chemically, or otherwise combined (e.g., in the same vial); compounds that are packaged together (e.g., as two separate objects in the same package (kit of parts) either for simultaneous, sequential or separate administration); or compounds that are provided separately but intended to be used together (e.g. the combination is expressly stated on the compound label or package insert). Suitably, the pharmaceutical combination consists of a medical compound formulated for oral administration and a medical compound formulated for subcutaneous injection. Suitably, the RTEL1 and FUBP1 inhibitors of the combination of the invention may be present in the same or in separate compositions. Suitably, the RTEL1 and FUBP1 inhibitors of the combination of the invention may be administered simultaneously, sequentially or separately. Suitably, RTEL1 and FUBP1 inhibitor of the combination of the invention are linked together by a physiologically labile linker such as defined in the present application. A suitable physiologically labile linker may comprises or consists of a DNA dinucleotide with a sequence selected from the group consisting of AA, AT, AC, AG, TA, TT, TC, TG, CA, CT, CC, CG, GA, GT, GC, or GG, where there is a phosphodiester linkage between the two DNA nucleosides. For example, the linker may by a CA dinucleotide.

### Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

The following is an example of an oligonucleotide motif (SEQ ID NO: 38) that is fully complementary to the target nucleic acid (SEQ ID NO: 12)

(SEQ ID NO: 12)
5′-CTTTGACCAGAGTATGTAAAATTCTC-3′
(SEQ ID NO: 38)
3′-AAACTGGTCTCATACATTTT-5′

### Compound

Herein, the term “compound” means any molecule capable of inhibition RTEL1 or FUBP1 expression or activity. Particular compounds of the combination of the invention are nucleic acid molecules, such as RNAi molecules or antisense oligonucleotides according to the invention or any conjugate comprising such a nucleic acid molecule. For example, herein the compound may be a nucleic acid molecule targeting RTEL1 or FUBP1, in particular an antisense oligonucleotide or a siRNA.

### Conjugate

The term conjugate as used herein refers to an oligonucleotide which is covalently linked to a non-nucleotide moiety (conjugate moiety or region C or third region).

Conjugation of the oligonucleotide (or nucleic acid molecule) of the combination of the invention to one or more non-nucleotide moieties may improve the pharmacology of the, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular, the conjugate may target the oligonucleotide to a specific organ, tissue or cell type and thereby enhance the effectiveness of the oligonucleotide in that organ, tissue or cell type. At the same time the conjugate may serve to reduce activity of the oligonucleotide in non-target cell types, tissues or organs, e.g. off target activity or activity in non-target cell types, tissues or organs. For siRNA nucleic acid molecules the conjugate moiety is most commonly covalently linked to the passenger strand of the siRNA, and for shRNA molecules the conjugate moiety would most commonly be linked to the end of the molecule which is furthest away from the contiguous nucleotide sequence of the shRNA. For antisense oligonucleotides the conjugate moiety can be covalently linked to any of the terminal ends, advantageously using a biocleavable linker such as a 2 to 5 phosphodiester linked DNA nucleosides.

WO 93/07883 and WO2013/033230 provides suitable conjugate moieties, which are hereby incorporated by reference. Further suitable conjugate moieties are those capable of binding to the asialoglycoprotein receptor (ASGPR). In particular, tri-valent N-acetylgalactosamine conjugate moieties are suitable for binding to the ASGPR, see for example US 2009/02398, WO 2014/076196, WO 2014/207232 and WO 2014/179620 (hereby incorporated by reference). Such conjugates serve to enhance uptake of the oligonucleotide to the liver while reducing its presence in the kidney, thereby increasing the liver/kidney ratio of a conjugated oligonucleotide compared to the unconjugated version of the same oligonucleotide.

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

In some embodiments, the conjugate is an antibody or an antibody fragment which has a specific affinity for a transferrin receptor, for example as disclosed in WO 2012/143379 herby incorporated by reference. In some embodiments, the non-nucleotide moiety is an antibody or antibody fragment, such as an antibody or antibody fragment that facilitates delivery across the blood-brain-barrier, in particular an antibody or antibody fragment targeting the transferrin receptor.

### Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the contiguous nucleotide sequence is included in the guide strand of an siRNA molecule. In some embodiments the contiguous nucleotide sequence is the part of an shRNA molecule which is 100% complementary to the target nucleic acid. In some embodiments the oligonucleotide comprises the contiguous nucleotide sequence, such as a F-G-F′ gapmer region, and may optionally comprise further nucleotide(s), for example a nucleotide linker region which may be used to attach a functional group (e.g. a conjugate group for targeting) to the contiguous nucleotide sequence. The nucleotide linker region may or may not be complementary to the target nucleic acid. In some embodiments, the nucleobase sequence of the antisense oligonucleotide is the contiguous nucleotide sequence. In some embodiments, the contiguous nucleotide sequence is at least 80%, such as at least 85%, such as at least 90%, such as at least 95%, such as at least 98% complementary to the target nucleic acid. In some embodiments, the contiguous nucleotide sequence is 100% complementary to the target nucleic acid.

### Gapmer

The antisense oligonucleotide, or contiguous nucleotide sequence thereof, may be a gapmer, also termed gapmer oligonucleotide or gapmer designs. The antisense gapmers are commonly used to inhibit a target nucleic acid via RNase H mediated degradation. In an embodiment of the invention the oligonucleotide is capable of recruiting RNase H. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides for use in the invention, or the contiguous nucleotide sequence thereof, may comprise a gapmer region of formula F-G-F′. In some embodiments, all internucleoside linkages between the nucleosides of the gapmer region of formula F-G-F′ are phosphorothioate internucleoside linkages.

The overall length of the gapmer design F-G-F′ may be, for example 12 to 32 nucleosides, such as 13 to 24, such as 14 to 22 nucleosides, Such as from 14 to17, such as 16 to18 nucleosides. In some embodiments, the overall length is 17 nucleosides. In some embodiments, the overall length is 17 nucleosides.

By way of example, the gapmer oligonucleotide of the present invention can be represented by the following formulae:

F₁₋₈-G₅₋₁₈-F′₁₋₈, such as

F₁₋₈-G₅₋₁₆-F′₁₋₈, or

F₁₋₈-G₇₋₁₆-F′₂₋₈, or

F₄₋₈-G₇₋₁₂-F′₂₋₈, or

F₄₋₆-G₇₋₁₁-F′₂₋₆

with the proviso that the overall length of the gapmer regions F-G-F′ is at least 12, such as at least 14 nucleotides in length.

In an aspect of the invention the antisense oligonucleotide or contiguous nucleotide sequence thereof consists of or comprises a gapmer of formula 5′-F-G-F′-3′, where region F and F′ independently comprise or consist of 1-8 nucleosides, of which 1-4 are 2′ sugar modified and defines the 5′ and 3′ end of the F and F′ region, and G is a region between 6 and 18, such as 6 and 16, nucleosides which are capable of recruiting RNaseH. In some embodiments the G region consists of DNA nucleosides.

In some embodiments, all the modified nucleosides of region F and F′ are beta-D-oxy LNA nucleosides. Further, region F or F′, or F and F′ may optionally comprise DNA nucleosides. Optionally, the flanking region F or F′, or both flanking regions F and F′ may comprise one or more DNA nucleosides (an alternating flank, see definition of the alternating flank for more details)

Regions F, G and F′ are further defined below and can be incorporated into the F-G-F′ formula.

### Gapmer-Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-18 contiguous DNA nucleosides, 5-17 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15, 16, 17 or 18 contiguous DNA nucleosides. Cytosine (C) DNA in the gap region may in some instances be methylated, such residues are either annotated as 5′-methyl-cytosine (meC or with an e instead of a c). Methylation of cytosine DNA in the gap is advantageous if cg dinucleotides are present in the gap to reduce potential toxicity, the modification does not have significant impact on efficacy of the oligonucleotides. 5′ substituted DNA nucleosides, such as 5′ methyl DNA nucleoside have been reported for use in DNA gap regions (EP 2 742 136).

In some embodiments, the gap region G may consist of 12 or less contiguous DNA nucleosides, such as of 7. 8. 9, 10, or 11 contiguous DNA nucleosides, such as 9, 10 or 11 contiguous DNA nucleosides.

One or more cytosine (C) DNA in the gap region may in some instances be methylated (e.g. when a DNA c is followed by a DNA g). Such residues are either annotated as 5-methyl-cytosine (^{me}C).

In some embodiments the gap region G may consist of 6, 7, 8, 9, 10, 11, 12, 13, 14, 15, 16, 17 or 18 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

### Gapmer-Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length or such as 4-6 contiguous nucleotides in length. In some embodiments, the length of region F is 4 contiguous nucleotides. In some embodiments, the length of region F is 5 contiguous nucleotides. In some embodiments, the length of region F is 6 contiguous nucleotides. Advantageously the 5′ most nucleoside of region F is a sugar modified nucleoside.

In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. In some embodiments, the length of region F′ is 2 contiguous nucleotides. In some embodiments, the length of region F′ is 3 contiguous nucleotides. In some embodiments, the length of region F′ is 4 contiguous nucleotides. In some embodiments, the length of region F′ is 5 contiguous nucleotides. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

Further gapmer designs are disclosed in WO2004/046160, WO2007/146511 and WO2008/113832, hereby incorporated by reference.

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

### HBV Infection

The term “hepatitis B virus infection” or “HBV infection” is commonly known in the art and refers to an infectious disease that is caused by the hepatitis B virus (HBV) and affects the liver. A HBV infection can be an acute or a chronic infection.

Some infected persons have no symptoms during the initial infection and some develop a rapid onset of sickness with vomiting, yellowish skin, tiredness, dark urine and abdominal pain (“Hepatitis B Fact sheet Nº204”.who.int. July 2014. Retrieved 4 Nov. 2014). Often these symptoms last a few weeks and can result in death. It may take 30 to 180 days for symptoms to begin. In those who get infected around the time of birth 90% develop a chronic hepatitis B infection while less than 10% of those infected after the age of five do (“Hepatitis B FAQs for the Public-Transmission”, U.S. Centers for Disease Control and Prevention (CDC), retrieved 2011 Nov. 29). Most of those with chronic disease have no symptoms; however, cirrhosis and liver cancer may eventually develop (Chang, 2007, Semin Fetal Neonatal Med, 12:160-167). These complications result in the death of 15 to 25% of those with chronic disease (“Hepatitis B Fact sheet Nº204”.who.int. July 2014, retrieved 4 Nov. 2014). Herein, the term “HBV infection” includes the acute and chronic hepatitis B infection. The term “HBV infection” also includes the asymptotic stage of the initial infection, the symptomatic stages, as well as the asymptotic chronic stage of the HBV infection.

Chronic hepatitis B virus (CHB) infection is a global disease burden affecting 248 million individuals worldwide. Approximately 686,000 deaths annually are attributed to HBV-related end-stage liver diseases and hepatocellular carcinoma (HCC) (GBD 2013; Schweitzer et al., 2015). WHO projected that without expanded intervention, the number of people living with CHB infection will remain at the current high levels for the next 40-50 years, with a cumulative 20 million deaths occurring between 2015 and 2030 (WHO 2016). CHB infection is not a homogenous disease with singular clinical presentation. Infected individuals have progressed through several phases of CHB-associated liver disease in their life; these phases of disease are also the basis for treatment with standard of care (SOC). Current guidelines recommend treating only selected CHB-infected individuals based on three criteria-serum ALT level, HBV DNA level, and severity of liver disease (EASL, 2017). This recommendation was due to the fact that SOC i.e. nucleos(t)ide analogs (NAs) and pegylated interferon-alpha (PEG-IFN), are not curative and must be administered for long periods of time thereby increasing their safety risks. NAs effectively suppress HBV DNA replication; however, they have very limited/no effect on other viral markers. Two hallmarks of HBV infection, hepatitis B surface antigen (HBsAg) and covalently closed circular DNA (cccDNA), are the main targets of novel drugs aiming for HBV cure. In the plasma of CHB individuals, HBsAg subviral (empty) particles outnumber HBV virions by a factor of 103 to 105 (Ganem & Prince, 2014); its excess is believed to contribute to immunopathogenesis of the disease, including inability of individuals to develop neutralizing anti-HBs antibody, the serological marker observed following resolution of acute HBV infection.

### High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides, for example Ome and MOE, as well as locked nucleic acids (LNA) (see e.g. Freier & Altmann; Nucl. Acid Res., 1997, 25, 4429-4443 and Uhlmann; Curr. Opinion in Drug Development, 2000, 3 (2), 293-213).

### Hybridization

The term “hybridizing” or “hybridizes” as used herein is to be understood as two nucleic acid strands (e.g. an oligonucleotide such as siRNA guide strand and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG °=−RTIn(K_{d}), where R is the gas constant and T is the absolute temperature. Therefore, a very low ΔG ° of the reaction between an oligonucleotide and the target nucleic acid reflects a strong hybridization between the oligonucleotide and target nucleic acid. ΔG ° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG ° is less than zero. ΔG ° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG ° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG ° values below-10 kcal for oligonucleotides that are 10-30 nucleotides in length. In some embodiments the degree or strength of hybridization is measured by the standard state Gibbs free energy ΔG °. The oligonucleotides may hybridize to a target nucleic acid with estimated ΔG ° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG ° value of −10 to −60 kcal, such as −12 to −40, such as from −15 to-30 kcal or −16 to −27 kcal such as −18 to −25 kcal.

### Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned nucleobases that are identical (a Match) between two sequences (in the contiguous nucleotide sequence of the compound for use in the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

### Inhibition of Expression

The term “Inhibition of expression” as used herein is to be understood as an overall term for an oligonucleotide's ability to inhibit the amount or the activity of a target (i.e. RTEL1 or FUBP1) in a target cell. Inhibition of activity may be determined by measuring the level of target pre-mRNA or target mRNA, or by measuring the level of target or target activity in a cell. Inhibition of expression may therefore be determined in vitro or in vivo.

Typically, inhibition of expression is determined by comparing the inhibition of activity due to the administration of an effective amount of the antisense oligonucleotide to the target cell and comparing that level to a reference level obtained from a target cell without administration of the antisense oligonucleotide (control experiment), or a known reference level (e.g. the level of expression prior to administration of the effective amount of the antisense oligonucleotide, or a predetermine or otherwise known expression level).

For example a control experiment may be an animal or person, or a target cell treated with a saline composition or a reference oligonucleotide (often a scrambled control).

The term inhibition or inhibit may also be referred as down-regulate, reduce, suppress, lessen, lower, the expression of a target.

The inhibition of expression may occur e.g. by degradation of pre-mRNA or mRNA (e.g. using RNase H recruiting oligonucleotides, such as gapmers).

### Inhibitor

The term “inhibitor” is known in the art and relates to a compound/substance or composition capable of fully or partially preventing or reducing the physiologic function (i.e. the activity) of (a) specific protein(s) (e.g. of FUBP1 or RTEL1).

In the context of the present invention, an “inhibitor” of FUBP1 is capable of preventing or reducing the activity/function of FUBP1, respectively, by preventing or reducing the expression of the FUBP1 gene products.

Similarly, in the context of the present invention, an “inhibitor” of RTEL1 is capable of preventing or reducing the activity/function of RTEL1, respectively, by preventing or reducing the expression of the RTEL1 gene product.

Thus, an inhibitor of FUBP1 or RTEL1 may lead to a decreased expression level of FUBP1 or RTEL1, respectively (e.g. decreased level of FUBP1 or RTEL1 mRNA, or of FUBP1 or RTEL protein, respectively) which is reflected in a decreased functionality (i.e. activity) of FUBP1 or RTEL1, wherein said function comprises the poly-A polymerase function. An inhibitor of FUBP1, in the context of the present invention, accordingly, may also encompass transcriptional repressors of FUBP1 expression that are capable of reducing the level of FUBP1. An inhibitor of RTEL1, in the context of the present invention, accordingly, may also encompass transcriptional repressors of RTEL1 expression that are capable of reducing the level of RTEL1. The term “inhibitor” also encompass pharmaceutically acceptable salt thereof. Preferred inhibitors are nucleic acid molecules.

### Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A).

In some embodiments of the invention the conjugate or oligonucleotide conjugate of the combination of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. In one embodiment, the linker between the oligonucleotide and the conjugate moiety is a physiologically labile linker composed of 2 to 5 consecutive phosphodiester linked nucleosides comprising at least two consecutive phosphodiester linkages at the 5′ or 3′ terminal of the contiguous nucleotide sequence of the antisense oligonucleotide.

In some embodiments, the physiologically labile linker comprises or consists of a DNA dinucleotide with a sequence selected from the group consisting of AA, AT, AC, AG, TA, TT, TC, TG, CA, CT, CC, CG, GA, GT, GC, or GG, where there is a phosphodiester linkage between the two DNA nucleosides and at least one further phosphodiester at the 5′ or 3′ end of the dinucleotide linking either the oligonucleotide of the nucleic acid molecule to the dinucleotide or the conjugate moiety to the dinucleotide. For example, the linker may by a CA dinucleotide. In some embodiments, the physiologically labile linker comprises or consists of a DNA trinucleotide of sequence AAA, AAT, AAC, AAG, ATA, ATT, ATC, ATG, ACA, ACT, ACC, ACG, AGA, AGT, AGC, AGG, TAA, TAT, TAC, TAG, TTA, TTT, TTC, TAG, TCA, TCT, TCC, TCG, TGA, TGT, TGC, TGG, CAA, CAT, CAC, CAG, CTA, CTG, CTC, CTT, CCA, CCT, CCC, CCG, CGA, CGT, CGC, CGG, GAA, GAT, GAC, CAG, GTA, GTT, GTC, GTG, GCA, GCT, GCC, GCG, GGA, GGT, GGC, or GGG, where there are phosphodiester linkages between the DNA nucleosides and potentially a further phosphodiester at the 5′ or 3′ end of the trinucleotide. Phosphodiester containing biocleavable linkers are described in more detail in WO 2014/076195 (hereby incorporated by reference). In a conjugate compound with a biocleavable linker at least about 50% of the conjugate moiety is cleaved from the oligonucleotide, such as at least about 60% cleaved, such as at least about 70% cleaved, such as at least about 80% cleaved, such as at least about 85% cleaved, such as at least about 90% cleaved, such as at least about 95% of the conjugate moiety is cleaved from the oligonucleotide cleaved when compared against a standard.

Region Y refers to linkers that are not necessarily biocleavable but primarily serve to covalently connect a conjugate moiety (region C or third region), to an oligonucleotide (region A or first region). The region Y linkers may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments the linker (region Y) is an amino alkyl, such as a C2-C36 amino alkyl group, including, for example C6 to C12 amino alkyl groups. In a preferred embodiment the linker (region Y) is a C6 amino alkyl group.

### LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₅-[region G]-[LNA]₁₋₅, wherein region G is as defined in the Gapmer region G definition.

### Locked Nucleic Acid Nucleosides (LNA Nucleoside)

A “LNA nucleoside” is a 2′-sugar modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75 (5) pp. 1569-81, Mitsuoka et al., Nucleic Acids Research 2009, 37 (4), 1225-1238, and Wan and Seth, J. Medical Chemistry 2016, 59, 9645-9667.

Particular examples of LNA nucleosides are presented in Scheme 1 (wherein B is as defined above).

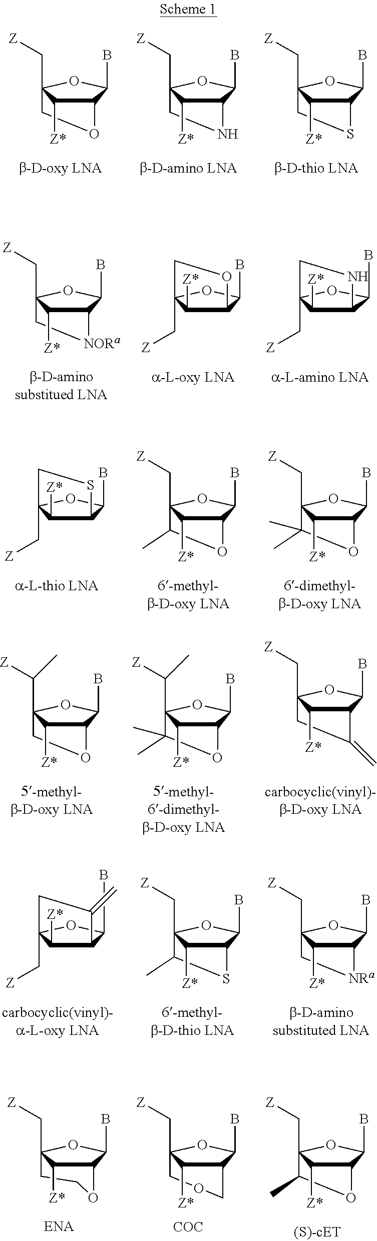

Particular LNA nucleosides are beta-D-oxy-LNA, 6′-methyl-beta-D-oxy LNA such as(S)-6′-methyl-beta-D-oxy-LNA (ScET) and ENA. A particularly advantageous LNA is beta-D-oxy-LNA.

### Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleoside. In some embodiments, wherein at least one of region F and F′, or both region F and F′ comprise at least one LNA nucleoside, the remaining nucleosides of region F and F′ are independently selected from the group consisting of MOE and LNA. In some embodiments, wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

### Modified Internucleoside Linkage

The term “modified internucleoside linkage” is defined as generally understood by the skilled person as linkages other than phosphodiester (PO) linkages, that covalently couples two nucleosides together. The oligonucleotides of the combination of the invention may therefore comprise one or more modified internucleoside linkages, such as a one or more phosphorothioate internucleoside linkages, or one or more phoshporodithioate internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the combination of the invention, for example within the gap region G of a gapmer oligonucleotide, as well as in regions of modified nucleosides, such as region F and F′.

In an embodiment, the oligonucleotide comprises one or more internucleoside linkages modified from the natural phosphodiester, such as one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 75%, such as at least 80% or such as at least 90% of the internucleoside linkages in the oligonucleotide, or contiguous nucleotide sequence thereof, are modified. In some embodiments all of the internucleoside linkages of the oligonucleotide, or contiguous nucleotide sequence thereof, are modified. It will be recognized that, in some embodiments the nucleosides which link the oligonucleotide of the combination of the invention to a non-nucleotide functional group, such as a conjugate, may be phosphodiester. In some embodiments all of the internucleoside linkages of the oligonucleotide, or contiguous nucleotide sequence thereof, are nuclease resistant internucleoside linkages.

With the oligonucleotide of the combination of the invention it is advantageous to use phosphorothioate internucleoside linkages.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Nuclease resistant linkages, such as phosphorthioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, where all the internucleoside linkages in region G may be phosphorothioate.

Advantageously, all the internucleoside linkages of the contiguous nucleotide sequence of the oligonucleotide are phosphorothioate, or all the internucleoside linkages of the oligonucleotide are phosphorothioate linkages.

Phosphorothioate linkages may exist in different tautomeric forms, for example as illustrated below:

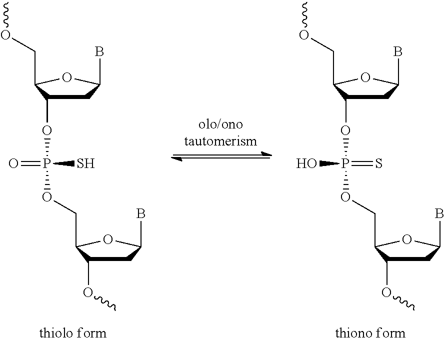

It is recognized that, as disclosed in EP 2 742 135, antisense oligonucleotides may comprise other internucleoside linkages (other than phosphodiester and phosphorothioate), for example alkyl phosphonate/methyl phosphonate internucleoside, which according to EP 2 742 135 may for example be tolerated in an otherwise DNA phosphorothioate the gap region.

### Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo) base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

### Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides and DNA nucleosides. The antisense oligonucleotide of the combination of the invention is advantageously a chimeric oligonucleotide.

### Modulation of Expression

The term “modulation of expression” as used herein is to be understood as an overall term for an oligonucleotide's ability to alter the amount of a target (i.e. RTEL1 or FUBP1) when compared to the amount of the target before administration of the oligonucleotide. Alternatively, modulation of expression may be determined by reference to a control experiment. It is generally understood that the control is an individual or target cell treated with a saline composition or an individual or target cell treated with a non-targeting oligonucleotide (mock).

One type of modulation is the ability of an oligonucleotide to inhibit, down-regulate, reduce, suppress, remove, stop, block, prevent, lessen, lower, avoid or terminate expression of a target (i.e. RTEL1 or FUBP1), e.g. by degradation of mRNA or blockage of transcription. Another type of modulation is an oligonucleotide's ability to restore, increase or enhance expression of a target, e.g. by repair of splice sites or prevention of splicing or removal or blockage of inhibitory mechanisms such as microRNA repression.

### MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₅₋₁₆-[MOE]₂₋₇, such as [MOE]₃₋₆-[Region G]-[MOE]₃₆, wherein region G is as defined in the Gapmer definition. MOE gapmers with a 5-10-5 design (MOE-DNA-MOE) have been widely used in the art.

### Naturally Occurring Variant

The term “naturally occurring variant” refers to variants of a gene or transcript (e.g. RTEL1 or FUBP1) which originate from the same genetic loci as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms (SNPs), and allelic variants. Based on the presence of the sufficient complementary sequence to the oligonucleotide, the oligonucleotide of the combination of the invention may therefore target the target nucleic acid and naturally occurring variants thereof.

In some embodiments, the naturally occurring variants have at least 95% such as at least 98% or at least 99% homology to a mammalian RTEL1 or FUBP1 target nucleic acid, such as a target nucleic acid of SEQ ID NO 1 and/or 2 for RTEL1, or SEQ ID NO: 247 and/or 251 for FUBP1. In some embodiments the RTEL1 naturally occurring variants have at least 99% homology to the human RTEL1 target nucleic acid of SEQ ID NO: 1. In some embodiments the FUBP1 naturally occurring variants have at least 99% homology to the human FUBP1 target nucleic acid of SEQ ID NO: 247. In some embodiments the naturally occurring variants are known polymorphisms.

### Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the combination of the invention are capable of recruiting a nuclease, particularly an endonuclease, preferably endoribonuclease (RNase), which recognizes RNA/DNA hybridization and effects cleavage of the RNA nucleic acid, such as RNase H. Examples of oligonucleotide designs which operate via nuclease mediated mechanisms are oligonucleotides which typically comprise a region of at least 5 or 6 consecutive DNA nucleosides and are flanked on one side or both sides by affinity enhancing nucleosides, for example gapmers, headmers and tailmers.

### Nucleic Acid Molecule (or “Oligonucleotide)

The term “nucleic acid molecule” or “therapeutic nucleic acid molecule” or “oligonucleotide” as used herein is defined as it is generally understood by the skilled person, as a molecule comprising two or more covalently linked nucleosides (i.e. a nucleotide sequence). Such covalently bound nucleosides may also be referred to as nucleic acid molecules or oligomers, which may be used interchangeably.

The nucleic acid molecule(s) referred to in the combination of the invention are generally therapeutic oligonucleotides below 50 nucleotides in length. The nucleic acid molecules may be or comprise a single stranded antisense oligonucleotide, or may be another oligomeric nucleic acid molecule, such as a CRISPR RNA, a siRNA, shRNA, an aptamer, or a ribozyme. Therapeutic nucleic acid molecules are commonly made in the laboratory by solid-phase chemical synthesis followed by purification and isolation. shRNA's are however often delivered to cells using lentiviral vectors from which are then transcribed to produce the single stranded RNA that will form a stem loop (hairpin) RNA structure that is capable of interacting with the RNA interference machinery (including the RNA-induced silencing complex (RISC)). When referring to a sequence of the nucleic acid molecule, reference is made to the sequence or order of nucleobase moieties, or modifications thereof, of the covalently linked nucleotides or nucleosides. The nucleic acid molecule of the combination of the invention is man-made, and is chemically synthesized, and is typically purified or isolated. In some embodiments the nucleic acid molecule of the combination of the invention is not a shRNA transcribed from a vector upon entry into the target cell. The nucleic acid molecule of the combination of the invention may comprise one or more modified nucleosides or nucleotides.

In some embodiments, the nucleic acid molecule of the combination of the invention comprises or consists of 12 to 60 nucleotides in length, such as from 13 to 50, such as from 14 to 40, such as from 15 to 30, such as from 16 to 22, such as from 16 to 18 or 15 to 17 contiguous nucleotides in length. Accordingly, the oligonucleotide of the present invention, in some embodiments, may have a length of 12-25 nucleotides. Alternatively, the oligonucleotide of the present invention, in some embodiments, may have a length of 15-22 nucleotides. In some embodiments, the nucleic acid molecule or contiguous nucleotide sequence thereof comprises or consists of 24 or less nucleotides, such as 22, such as 20 or less nucleotides, such as 18 or less nucleotides, such as 14, 15, 16 or 17 nucleotides. It is to be understood that any range given herein includes the range endpoints. Accordingly, if a nucleic acid molecule is said to include from 12 to 30 nucleotides, both 12 and 30 nucleotides are included. In some embodiments, the contiguous nucleotide sequence comprises or consists of at least 10, such as 11, 12, 13, 14, 15, 16, 17, 18, 19, 20, 21, 22, 23, 24, 25, 26, 27, 28, 29 or 30 contiguous nucleotides in length

The nucleic acid molecule(s) are for modulating the expression of a target nucleic acid in a mammal. In some embodiments the nucleic acid molecules, such as for siRNAs, shRNAs and antisense oligonucleotides, are typically for inhibiting the expression of a target nucleic acid(s). In one embodiment of the invention the nucleic acid molecule is selected from a RNAi agent, such as a siRNA or shRNA. In another embodiment the nucleic acid molecule is a single stranded antisense oligonucleotide, such as a high affinity modified antisense oligonucleotide interacting with RNaseH.

In some embodiments the nucleic acid molecule of the combination of the invention may comprise one or more modified nucleosides or nucleotides, such as 2′ sugar modified nucleosides.

In some embodiments the nucleic acid molecule comprises phosphorothioate internucleoside linkages.

In some embodiments the nucleic acid molecule may be conjugated to non-nucleosidic moieties (conjugate moieties).

A library of nucleic acid molecules is to be understood as a collection of variant nucleic acid molecules. The purpose of the library of nucleic acid molecules can vary. In some embodiments, the library of nucleic acid molecules is composed of oligonucleotides with overlapping nucleobase sequence targeting one or more mammalian target nucleic acids (i.e. RTEL1 or FUBP1) with the purpose of identifying the most potent sequence within the library of nucleic acid molecules. In some embodiments, the library of nucleic acid molecules is a library of nucleic acid molecule design variants (child nucleic acid molecules) of a parent or ancestral nucleic acid molecule, wherein the nucleic acid molecule design variants retaining the core nucleobase sequence of the parent nucleic acid molecule.

### Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

### Nucleotides and Nucleosides

Nucleotides and nucleosides are the building blocks of oligonucleotides and polynucleotides, and for the purposes of the present invention include both naturally occurring and non-naturally occurring nucleotides and nucleosides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

### Patient

For the purposes of the present invention the “subject” (or “patient”) may be a vertebrate. In context of the present invention, the term “subject” includes both humans and other animals, particularly mammals, and other organisms. Thus, the herein provided means and methods are applicable to both human therapy and veterinary applications. Accordingly, herein the subject may be an animal such as a mouse, rat, hamster, rabbit, guinea pig, ferret, cat, dog, chicken, sheep, bovine species, horse, camel, or primate. Preferably, the subject is a mammal. More preferably the subject is human. In some embodiments, the patient is suffering from a disease as referred to herein, such as HBV infection. In some embodiments, the patient is susceptible to said disease.

### Pharmaceutical Composition

In a further aspect, the invention provides pharmaceutical compositions comprising an oligonucleotide for use in the invention and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts.

The invention provides for a pharmaceutical composition according to the invention, wherein the pharmaceutical composition comprises the oligonucleotide useful in the invention, and an aqueous diluent or solvent.

The invention provides for a solution, such as a phosphate buffered saline solution of the oligonucleotide of the combination of the invention. Suitably the solution, such as phosphate buffered saline solution, of the invention is a sterile solution.

WO 2007/031091 provides suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in W02007/031 091.

Oligonucleotides for use in the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

In some embodiments, the oligonucleotide or oligonucleotide conjugate useful in the invention is a prodrug. In particular, with respect to oligonucleotide conjugates, the conjugate moiety of the oligonucleotide is cleaved once the prodrug is delivered to the site of action, e.g. the target cell.

### Pharmaceutically Acceptable Salts

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition, these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

### Prevention

Herein the term “preventing”, “prevention” or “prevents” relates to a prophylactic treatment, i.e. to a measure or procedure the purpose of which is to prevent, rather than to cure a disease. Prevention means that a desired pharmacological and/or physiological effect is obtained that is prophylactic in terms of completely or partially preventing a disease or symptom thereof. Accordingly, herein “preventing a HBV infection” includes preventing a HBV infection from occurring in a subject, and preventing the occurrence of symptoms of a HBV infection. In the present invention in particular the prevention of HBV infection in children from HBV infected mothers are contemplated. Also contemplated is the prevention of an acute HBV infection turning into a chronic HBV infection.

### Region D′ or D″ in an Oligonucleotide

The oligonucleotide of the combination of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs (e.g. gapmer regions) within a single oligonucleotide.

In one embodiment the oligonucleotide of the combination of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₆-F′₂₋₈

D′-F-G-F′, in particular D′₁₋₃-F₁₋₈-G₅₋₁₆-F′₂₋₈

F-G-F′-D″, in particular F₁₋₈-G₅₋₁₆-F′₂₋₈-D″₁₋₃

D′-F-G-F′-D″, in particular D′₁₋₃-F₁₋₈-G₅₋₁₆-F′₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

### RNAi Molecules

Herein, the term “RNA interference (RNAi) molecule” refers to short double-stranded RNA based oligonucleotide capable of inducing RNA-dependent gene silencing via the RNA-induced silencing complex (RISC) in a cell's cytoplasm, where they interact with the catalytic RISC component argonaute. The RNAi molecule modulates. e g., inhibits, the expression of the target nucleic acid in a cell. e.g. a cell within a subject, such as a mammalian subject. One type of RNAi molecule is a small interfering RNA (siRNA), which is a double-stranded RNA molecule composed of two complementary oligonucleotides, where the binding of one strand to complementary mRNA after transcription, leads to its degradation and loss of translation. A small hairpin RNA (shRNA) is a single stranded RNA-based oligonucleotide that forms a stem loop (hairpin) structure which is able to reduce mRNA via the DICER and RNA reducing silencing complex (RISC). RNAi molecules can be designed based on the sequence of the gene of interest (target nucleic acid). Corresponding RNAi can then be synthesized chemically or by in vitro transcription, or expressed from a vector or PCR product.

### RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO 01/23613 (hereby incorporated by reference). For use in determining RHase H activity, recombinant human RNase H1 is available from Creative Biomart® (Recombinant Human RNASEH1 fused with His tag expressed in E. coli).

shRNA

Short hairpin RNA or shRNA molecules are generally between 40 and 70 nucleotides in length, such as between 45 and 65 nucleotides in length, such as 50 and 60 nucleotides in length, and form a stem loop (hairpin) RNA structure, which interacts with the endonuclease known as Dicer which is believed to processes dsRNA into 19-23 base pair short interfering RNAs with characteristic two base 3′ overhangs which are then incorporated into an RNA-induced silencing complex (RISC). Upon binding to the appropriate target mRNA, one or more endonucleases within the RISC cleave the target to induce silencing. RNAi oligonucleotides may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA.

In some embodiments shRNA nucleic acid molecules comprise one or more phosphorothioate internucleoside linkages. In RNAi molecules phosphorothioate internucleoside linkages may reduce or the nuclease cleavage in RICS it is therefore advantageous that not al internucleoside linkages in the stem loop of the shRNA molecule are modified. Phosphorothioate internucleoside linkages can advantageously be place in the 3′ and/or 5′ end of the stem loop of the shRNA molecule, in particular in the of the part of the molecule that is not complementary to the target nucleic acid (e.g. the sense stand or passenger strand in an siRNA molecule). The region of the shRNA molecule that is complementary to the target nucleic acid may however also be modified in the first 2 to 3 internucleoside linkages in the part that is predicted to become the 3′ and/or 5′ terminal following cleavage by Dicer.

siRNA

The term siRNA refers to a small interfering ribonucleic acid RNAi molecule. It is a class of double-stranded RNA molecules, also known in the art as short interfering RNA or silencing RNA. siRNAs typically comprise a sense strand (also referred to as a passenger strand) and an antisense strand (also referred to as the guide strand), wherein each strand are of 17-30 nucleotides in length, typically 19-25 nucleosides in length, wherein the antisense strand is complementary, such as at least 95% complementary, such as fully complementary, to the target nucleic acid (suitably a mature mRNA sequence), and the sense strand is complementary to the antisense strand so that the sense strand and antisense strand form a duplex or duplex region. siRNA strands may form a blunt ended duplex, or advantageously the sense and antisense strand 3′ ends may form a 3′ overhang of e.g. 1, 2 or 3 nucleosides to resemble the product produced by Dicer, which forms the RISC substrate in vivo. Effective extended forms of Dicer substrates have been described in U.S. Pat. Nos. 8,349,809 and 8,513,207, hereby incorporated by reference. In some embodiments, both the sense strand and antisense strand have a 2 nt 3′ overhang. The duplex region may therefore be, for example 17-25 nucleotides in length, such as 21-23 nucleotide in length.

Once inside a cell the antisense strand is incorporated into the RISC complex which mediate target degradation or target inhibition of the target nucleic acid. siRNAs typically comprise modified nucleosides in addition to RNA nucleosides. In one embodiment the siRNA molecule may be chemically modified using modified internucleotide linkages and 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA. In particular 2′fluoro, 2′-O-methyl or 2′-O-methoxyethyl may be incorporated into siRNAs.

In some embodiments all of the nucleotides of an siRNA sense (passenger) strand may be modified with 2′ sugar modified nucleosides such as LNA (see WO2004/083430, WO2007/085485 for example). In some embodiments the passenger stand of the siRNA may be discontinuous (see WO2007/107162 for example). The incorporation of thermally destabilizing nucleotides occurring at a seed region of the antisense strand of siRNAs have been reported as useful in reducing off-target activity of siRNAs (see WO2018/098328 for example). Suitably the siRNA comprises a 5′ phosphate group or a 5′-phosphate mimic at the 5′ end of the antisense strand. In some embodiments the 5′ end of the antisense strand is a RNA nucleoside.

In one embodiment, the siRNA molecule further comprises at least one phosphorothioate or methylphosphonate internucleoside linkage. The phosphorothioaie or methylphosphonate internucleoside linkage may be at the 3′-terminus one or both strand (e.g., the antisense strand; or the sense strand); or the phosphorothioate or methylphosphonate internucleoside linkage may be at the 5′-terminus of one or both strands (e.g., the antisense strand; or the sense strand); or the phosphorothioate or methylphosphonate internucleoside linkage may be at the both the 5′- and 3′-terminus of one or both strands (e.g., the antisense strand; or the sense strand). In some embodiments the remaining internucleoside linkages are phosphodiester linkages. In some embodiments siRNA molecules comprise one or more phosphorothioate internucleoside linkages. In siRNA molecules phosphorothioate internucleoside linkages may reduce or the nuclease cleavage in RICS, it is therefore advantageous that not all internucleoside linkages in the antisense strand are modified.

The siRNA molecule may further comprise a ligand. In some embodiments, the ligand is conjugated to the 3′ end of the sense strand.

For biological distribution, siRNAs may be conjugated to a targeting ligand, and/or be formulated into lipid nanoparticles, for example.

Other aspects of the invention relate to pharmaceutical compositions comprising these dsRNA, such as siRNA molecules suitable for therapeutic use, and methods of reducing the expression of the target gene by administering the dsRNA molecules such as siRNAs of the combination of the invention, e.g., for the treatment of various disease conditions as disclosed herein.

### Sugar Modifications

The oligonucleotide of the combination of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′—OH group naturally found in DNA and RNA nucleosides. Substituents may, for example be introduced at the 2′, 3′, 4′ or 5′ positions.

### Target Cell

The term a “target cell” as used herein refers to a cell which is expressing the target nucleic acid. For the therapeutic use of the present invention it is advantageous if the target cell is infected with HBV. In some embodiments the target cell may be in vivo or in vitro. In some embodiments the target cell is a mammalian cell such as a rodent cell, such as a mouse cell or a rat cell, or a woodchuck cell or a primate cell such as a monkey cell (e.g. a cynomolgus monkey cell) or a human cell.

In preferred embodiments the target cell expresses RTEL1 and/or FUBP1 mRNA, such as pre-mRNA or mature mRNA. Preferably, the target cell expresses both RTEL1 and FUBP1 mRNA, such as pre-mRNA or mature mRNA. The poly A tail of RTEL1 and/or FUBP1 mRNA is typically disregarded for antisense oligonucleotide targeting.

Typically, the target cell expresses the RTEL1 mRNA, such as the RTEL1 pre-mRNA or RTEL1 mature mRNA. For experimental evaluation a target cell may be used which expresses a nucleic acid which comprises a target sequence, such as the human RTEL1 pre-mRNA, e.g. SEQ ID NO: 1. The poly A tail of RTEL1 mRNA is typically disregarded for antisense oligonucleotide targeting.

The combination of the invention is typically capable of inhibiting the expression of the RTEL1 target nucleic acid in a cell which is expressing the RTEL1 target nucleic acid (a target cell), for example either in vivo or in vitro.

Typically, the target cell also expresses the FUBP1 mRNA, such as the FUBP1 pre-mRNA or FUBP1 mature mRNA. For example, the target cell expresses the human FUBP1 pre-mRNA, e.g. SEQ ID NO 247, or human FUBP1 mature mRNA comprising exon 14, such as SEQ ID NO: 249 or 250) or exon 20 of SEQ ID NO 247. For experimental evaluation a target cell may be used which expresses a nucleic acid which comprises a target sequence. The poly A tail of FUBP1 mRNA is typically disregarded for antisense oligonucleotide targeting. The combination of the invention is typically capable of inhibiting the expression of the FUBP1 target nucleic acid in a target cell which is expressing the FUBP1 target nucleic acid, for example either in vivo or in vitro.

Further, the target cell may be a hepatocyte. In one embodiment the target cell is HBV infected primary human hepatocytes, either derived from HBV infected individuals or from a HBV infected mouse with a humanized liver (PhoenixBio, PXB-mouse).

In accordance with the present invention, the target cell may be infected with HBV. Further, the target cell may comprise HBV cccDNA. Thus, the target cell preferably comprises RTEL1 and/or FUBP1 mRNA, such as pre-mRNA or mature mRNA, and HBV cccDNA. More preferably, the target cell comprises both RTEL1 and FUBP1 mRNA, such as pre-mRNA or mature mRNA, and HBV cccDNA.

### RTEL1 Target Nucleic Acid

According to the present invention, the target nucleic acid is a nucleic acid which encodes mammalian RTEL1 and may for example be a gene, a RNA, a mRNA, and pre-mRNA, a mature mRNA or a cDNA sequence. The target may therefore be referred to as an RTEL1 target nucleic acid.

The oligonucleotide for use in the invention may for example target exon regions of a mammalian RTEL1 (in particular siRNA and shRNA target exon regions, but also antisense oligonucleotides), or may for example target intron region in the RTEL1 pre-mRNA (in particular antisense oligonucleotides target intron regions). The human RTEL1 gene encodes 15 transcripts of these 7 are protein coding and therefore potential nucleic acid targets.

Table 1 lists predicted exon and intron regions of the 7 transcripts, as positioned on the human RTEL1 premRNA of SEQ ID NO: 1. It is understood that the oligonucleotides for use in the invention can target the mature mRNA sequence of one or more of the listed transcripts in table 1.

TABLE 1
Transcript-, exonic- and intronic regions in the human RTEL1 premRNA (SEQ
ID NO: 1) for the different protein coding RTEL1 mRNA transcripts

| | Transcript region | | Exonic regions | | | Intron regions | | |
|---|---|---|---|---|---|---|---|---|
| Transcript ID | start | end | Exon | start | end | intron | start | end |
| RTEL1-205 | 1 | 38444 | 1 | 1 | 657 | 1 | 657 | 1424 |
| ENST00000370018 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4737 |
| | | | 5 | 4737 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | 10 | 16284 | 20336 |
| | | | 11 | 20336 | 20374 | 11 | 20374 | 20459 |
| | | | 12 | 20459 | 20537 | 12 | 20537 | 22040 |
| | | | 13 | 22040 | 22137 | 13 | 22137 | 22855 |
| | | | 14 | 22855 | 22910 | 14 | 22910 | 27714 |
| | | | 15 | 27714 | 27788 | 15 | 27788 | 27982 |
| | | | 16 | 27982 | 28063 | 16 | 28063 | 29829 |
| | | | 17 | 29829 | 29961 | 17 | 29961 | 30128 |
| | | | 18 | 30128 | 30241 | 18 | 30241 | 30330 |
| | | | 19 | 30330 | 30370 | 19 | 30370 | 30492 |
| | | | 20 | 30492 | 30577 | 20 | 30577 | 30719 |
| | | | 21 | 30719 | 30796 | 21 | 30796 | 31246 |
| | | | 22 | 31246 | 31323 | 22 | 31323 | 31693 |
| | | | 23 | 31693 | 31839 | 23 | 31839 | 31941 |
| | | | 24 | 31941 | 32056 | 24 | 32056 | 32278 |
| | | | 25 | 32278 | 32401 | 25 | 32401 | 32485 |
| | | | 26 | 32485 | 32632 | 26 | 32632 | 32996 |
| | | | 27 | 32996 | 33138 | 27 | 33138 | 33933 |
| | | | 28 | 33933 | 34028 | 28 | 34028 | 34996 |
| | | | 29 | 34996 | 35194 | 29 | 35194 | 35334 |
| | | | 30 | 35334 | 35474 | 30 | 35474 | 36563 |
| | | | 31 | 36563 | 36679 | 31 | 36679 | 36932 |
| | | | 32 | 36932 | 37165 | 32 | 37165 | 37257 |
| | | | 33 | 37257 | 37412 | 33 | 37412 | 37519 |
| | | | 34 | 37519 | 37671 | 34 | 37671 | 37969 |
| | | | 35 | 37969 | 38444 | | | |
| RTEL1-203 | 485 | 38433 | 1 | 485 | 657 | 1 | 657 | 1424 |
| ENST00000360203 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4737 |
| | | | 5 | 4737 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | 10 | 16284 | 20336 |
| | | | 11 | 20336 | 20374 | 11 | 20374 | 20459 |
| | | | 12 | 20459 | 20537 | 12 | 20537 | 22040 |
| | | | 13 | 22040 | 22137 | 13 | 22137 | 22855 |
| | | | 14 | 22855 | 22910 | 14 | 22910 | 27714 |
| | | | 15 | 27714 | 27788 | 15 | 27788 | 27982 |
| | | | 16 | 27982 | 28063 | 16 | 28063 | 29829 |
| | | | 17 | 29829 | 29961 | 17 | 29961 | 30128 |
| | | | 18 | 30128 | 30241 | 18 | 30241 | 30330 |
| | | | 19 | 30330 | 30370 | 19 | 30370 | 30492 |
| | | | 20 | 30492 | 30577 | 20 | 30577 | 30719 |
| | | | 21 | 30719 | 30796 | 21 | 30796 | 31246 |
| | | | 22 | 31246 | 31323 | 22 | 31323 | 31693 |
| | | | 23 | 31693 | 31839 | 23 | 31839 | 31941 |
| | | | 24 | 31941 | 32056 | 24 | 32056 | 32278 |
| | | | 25 | 32278 | 32401 | 25 | 32401 | 32485 |
| | | | 26 | 32485 | 32632 | 26 | 32632 | 32996 |
| | | | 27 | 32996 | 33138 | 27 | 33138 | 33933 |
| | | | 28 | 33933 | 34028 | 28 | 34028 | 34996 |
| | | | 29 | 34996 | 35194 | 29 | 35194 | 35334 |
| | | | 30 | 35334 | 35474 | 30 | 35474 | 36563 |
| | | | 31 | 36563 | 36679 | 31 | 36679 | 36932 |
| | | | 32 | 36932 | 37165 | 32 | 37165 | 37257 |
| | | | 33 | 37257 | 37412 | 33 | 37412 | 37519 |
| | | | 34 | 37519 | 37841 | 34 | 37841 | 37969 |
| | | | 35 | 37969 | 38433 | | | |
| RTEL1-212 | 482 | 38171 | 1 | 482 | 657 | 1 | 657 | 1424 |
| ENST00000508582 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4665 |
| | | | 5 | 4665 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | 10 | 16284 | 20336 |
| | | | 11 | 20336 | 20374 | 11 | 20374 | 20459 |
| | | | 12 | 20459 | 20537 | 12 | 20537 | 22040 |
| | | | 13 | 22040 | 22137 | 13 | 22137 | 22855 |
| | | | 14 | 22855 | 22910 | 14 | 22910 | 27714 |
| | | | 15 | 27714 | 27788 | 15 | 27788 | 27982 |
| | | | 16 | 27982 | 28063 | 16 | 28063 | 29829 |
| | | | 17 | 29829 | 29961 | 17 | 29961 | 30128 |
| | | | 18 | 30128 | 30241 | 18 | 30241 | 30330 |
| | | | 19 | 30330 | 30370 | 19 | 30370 | 30492 |
| | | | 20 | 30492 | 30577 | 20 | 30577 | 30719 |
| | | | 21 | 30719 | 30796 | 21 | 30796 | 31246 |
| | | | 22 | 31246 | 31323 | 22 | 31323 | 31693 |
| | | | 23 | 31693 | 31839 | 23 | 31839 | 31941 |
| | | | 24 | 31941 | 32056 | 24 | 32056 | 32278 |
| | | | 25 | 32278 | 32401 | 25 | 32401 | 32485 |
| | | | 26 | 32485 | 32632 | 26 | 32632 | 32996 |
| | | | 27 | 32996 | 33138 | 27 | 33138 | 33933 |
| | | | 28 | 33933 | 34028 | 28 | 34028 | 34996 |
| | | | 29 | 34996 | 35194 | 29 | 35194 | 35334 |
| | | | 30 | 35334 | 35474 | 30 | 35474 | 36563 |
| | | | 31 | 36563 | 36679 | 31 | 36679 | 36932 |
| | | | 32 | 36932 | 37165 | 32 | 37165 | 37257 |
| | | | 33 | 37257 | 37412 | 33 | 37412 | 37519 |
| | | | 34 | 37519 | 37671 | 34 | 37671 | 37969 |
| | | | 35 | 37969 | 38171 | | | |
| RTEL1-201 | 505 | 38434 | 1 | 505 | 650 | 1 | 650 | 3489 |
| ENST00000318100 | | | 2 | 3489 | 3687 | 2 | 3687 | 4041 |
| | | | 3 | 4041 | 4134 | 3 | 4134 | 4737 |
| | | | 4 | 4737 | 4818 | 4 | 4818 | 5020 |
| | | | 5 | 5020 | 5080 | 5 | 5080 | 8195 |
| | | | 6 | 8195 | 8270 | 6 | 8270 | 9660 |
| | | | 7 | 9660 | 9744 | 7 | 9744 | 14747 |
| | | | 8 | 14747 | 14812 | 8 | 14812 | 16131 |
| | | | 9 | 16131 | 16284 | 9 | 16284 | 20336 |
| | | | 10 | 20336 | 20374 | 10 | 20374 | 20459 |
| | | | 11 | 20459 | 20537 | 11 | 20537 | 22040 |
| | | | 12 | 22040 | 22137 | 12 | 22137 | 22855 |
| | | | 13 | 22855 | 22910 | 13 | 22910 | 27714 |
| | | | 14 | 27714 | 27788 | 14 | 27788 | 27982 |
| | | | 15 | 27982 | 28063 | 15 | 28063 | 29829 |
| | | | 16 | 29829 | 29961 | 16 | 29961 | 30128 |
| | | | 17 | 30128 | 30241 | 17 | 30241 | 30330 |
| | | | 18 | 30330 | 30370 | 18 | 30370 | 30492 |
| | | | 19 | 30492 | 30577 | 19 | 30577 | 30719 |
| | | | 20 | 30719 | 30796 | 20 | 30796 | 31246 |
| | | | 21 | 31246 | 31323 | 21 | 31323 | 31693 |
| | | | 22 | 31693 | 31839 | 22 | 31839 | 31941 |
| | | | 23 | 31941 | 32056 | 23 | 32056 | 32278 |
| | | | 24 | 32278 | 32401 | 24 | 32401 | 32485 |
| | | | 25 | 32485 | 32632 | 25 | 32632 | 32996 |
| | | | 26 | 32996 | 33138 | 26 | 33138 | 33933 |
| | | | 27 | 33933 | 34028 | 27 | 34028 | 34996 |
| | | | 28 | 34996 | 35194 | 28 | 35194 | 35334 |
| | | | 29 | 35334 | 35474 | 29 | 35474 | 36563 |
| | | | 30 | 36563 | 36679 | 30 | 36679 | 36932 |
| | | | 31 | 36932 | 37165 | 31 | 37165 | 37257 |
| | | | 32 | 37257 | 37412 | 32 | 37412 | 37519 |
| | | | 33 | 37519 | 37671 | 33 | 37671 | 37969 |
| | | | 34 | 37969 | 38434 | | | |
| RTEL1-202 | 551 | 16284 | 1 | 551 | 650 | 1 | 650 | 1424 |
| ENST00000356810 | | | 2 | 1424 | 1695 | 2 | 1695 | 3489 |
| | | | 3 | 3489 | 3687 | 3 | 3687 | 4041 |
| | | | 4 | 4041 | 4134 | 4 | 4134 | 4587 |
| | | | 5 | 4587 | 4818 | 5 | 4818 | 5020 |
| | | | 6 | 5020 | 5080 | 6 | 5080 | 8195 |
| | | | 7 | 8195 | 8270 | 7 | 8270 | 9660 |
| | | | 8 | 9660 | 9744 | 8 | 9744 | 14747 |
| | | | 9 | 14747 | 14812 | 9 | 14812 | 16131 |
| | | | 10 | 16131 | 16284 | | | |
| RTEL1-206 | 30530 | 33067 | 1 | 30530 | 30577 | 1 | 30577 | 30719 |
| ENST00000425905 | | | 2 | 30719 | 30796 | 2 | 30796 | 31246 |
| | | | 3 | 31246 | 31323 | 3 | 31323 | 31941 |
| | | | 4 | 31941 | 32056 | 4 | 32056 | 32278 |
| | | | 5 | 32278 | 32401 | 5 | 32401 | 32485 |
| | | | 6 | 32485 | 32632 | 6 | 32632 | 32996 |
| | | | 7 | 32996 | 33067 | | | |
| RTEL1-214 | 811 | 3653 | 1 | 811 | 943 | 1 | 943 | 1424 |
| ENST00000646389 | | | 2 | 1424 | 1695 | 2 | 1695 | 3489 |
| | | | 3 | 3489 | 3653 | | | |

Suitably, the target nucleic acid encodes an RTEL1 protein, in particular mammalian RTEL1, such as human RTEL1 (See for example tables 2 and 3) which provides the pre-mRNA 5 sequences for human and monkey, RTEL1.

In some embodiments, the target nucleic acid is selected from SEQ ID NO: 1 and/or 2 or naturally occurring variants thereof (e.g. sequences encoding a mammalian RTEL 1 protein in table 1).

If employing the combination of the invention in research or diagnostics the target nucleic acid 10 may be a cDNA or a synthetic nucleic acid derived from DNA or RNA.

For in vivo or in vitro application, the combination of the invention is typically capable of inhibiting the expression of the RTEL1 target nucleic acid in a cell which is expressing the RTEL1 target nucleic acid. The contiguous sequence of nucleobases of the oligonucleotide of the combination of the invention is typically complementary to the RTEL1 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). The target nucleic acid may, in some embodiments, be a RNA or DNA, such as a messenger RNA, such as a mature mRNA (e.g. the exonic regions of the transcripts listed in table 1) or a pre-mRNA.

In some embodiments the target nucleic acid is a RNA or DNA which encodes mammalian RTEL1 protein, such as human RTEL1, e.g. the human RTEL1 mRNA sequence, such as that disclosed as SEQ ID NO 1. Further information on exemplary target nucleic acids is provided in tables 2 and 3.

TABLE 2
Genome and assembly information for RTEL1 across species.

| | | | Genomic | | | |
|---|---|---|---|---|---|---|
| | | | coordinates | | | ensembl |
| Species | Chr. | Strand | Start | End | Assembly | gene_id |
| Human | 20 | fwd | 63657810 | 63696253 | GRCh38.p12 | ENSG00000258366 |
| Cynomolgus | 10 | fwd | 95853726 | 95890939 | Macaca_fascicularis_5.0 | ENSMFAG00000043680 |
| monkey | | | | | | |

Fwd = forward strand. The genome coordinates provide the pre-mRNA sequence (genomic sequence). The NCBI reference provides the mRNA sequence (cDNA sequence).

TABLE 3
Sequence details for RTEL1 across species.

| Species | RNA type | Length (nt) | SEQ ID NO |
|---|---|---|---|
| Human | premRNA | 38444 | 1 |
| Monkey | premRNA | 37214 | 2 |

Note SEQ ID NO 2 comprises regions of multiple NNNNs, where the sequencing has been unable to accurately refine the sequence, and a degenerate sequence is therefore included. For the avoidance of doubt the compounds for use in the invention are complementary to the actual target sequence and are not therefore degenerate compounds.

In some embodiments, the target nucleic acid is SEQ ID NO 1.

In some embodiments, the target nucleic acid is SEQ ID NO 2.

### FUBP1 Target Nucleic Acid

According to the present invention, the target nucleic acid is a nucleic acid, which encodes mammalian FUBP1 and may for example be a gene, a RNA, an mRNA, and pre-mRNA, a mature mRNA or a cDNA sequence. The target may therefore be referred to as a FUBP1 target nucleic acid.

Suitably, the target nucleic acid encodes a FUBP1 protein, in particular mammalian FUBP1, such as the human FUBP1 gene encoding pre-mRNA or mRNA sequences provided herein as SEQ ID NO: 247, 249 and/or 250. SEQ ID NO: 247 is sequence of the human FUBP1 pre-mRNA. SEQ ID NO: 249 and 250 are sequences of human FUBP1 mRNAs.

The nucleic acid molecules of the combination of the invention may for example target exon regions of a mammalian FUBP1 (in particular siRNA and shRNA, but also antisense oligonucleotides), or may for example target any intron region in the FUBP1 pre-mRNA (in particular antisense oligonucleotides). Table 4 lists predicted exon and intron regions of SEQ ID NO: 247.

TABLE 4
Exon and intron regions in the human FUBP1 pre-mRNA.

| Exonic regions in the | | | Intronic regions in the | | |
|---|---|---|---|---|---|
| human FUBP1 premRNA | | | human FUBP1 premRNA | | |
| (SEQ ID NO 247) | | | (SEQ ID NO 247) | | |
| ID | start | end | ID | start | end |
| E1 | 19 | 226 | I1 | 227 | 9095 |
| E2 | 9096 | 9186 | I2 | 9187 | 10907 |
| E3 | 10908 | 10946 | I3 | 10947 | 11444 |
| E4 | 11445 | 11484 | I4 | 11485 | 12009 |
| E5 | 12010 | 12062 | I5 | 12063 | 12155 |
| E6 | 12156 | 12227 | I6 | 12228 | 12359 |
| E7 | 12360 | 12417 | I7 | 12418 | 13879 |
| E8 | 13880 | 14042 | I8 | 14043 | 14142 |
| E9 | 14143 | 14241 | I9 | 14242 | 14363 |
| E10 | 14364 | 14465 | I10 | 14466 | 14754 |
| E11 | 14755 | 14857 | I11 | 14858 | 14948 |
| E12 | 14949 | 15049 | I12 | 15050 | 15395 |
| E13 | 15396 | 15537 | I13 | 15538 | 16180 |
| E14 | 16181 | 16341 | I14 | 16342 | 18615 |
| E15 | 18616 | 18767 | I15 | 18768 | 18847 |
| E16 | 18848 | 18927 | I16 | 18928 | 22410 |
| E17 | 22411 | 22539 | I17 | 22540 | 23781 |
| E18 | 23782 | 23856 | I18 | 23857 | 29810 |
| E19 | 29811 | 29956 | I19 | 29957 | 30196 |
| E20 | 30197 | 30706 | | | |

Suitably, the target nucleic acid encodes a FUBP1 protein, in particular mammalian FUBP1, such as human FUBP1 (See for example Tables 5 and 6) which provides the genomic sequence, the mature mRNA and pre-mRNA sequences for human, monkey and mouse 5 FUBP1).

In some embodiments, the target nucleic acid may be a cynomolgus monkey FUBP1 nucleic acid, such as an mRNA or pre-mRNA.

In some embodiments, the target nucleic acid may be a mouse FUBP1 nucleic acid, such as a mRNA or pre-mRNA.

In some embodiments, the target nucleic acid is selected from the group consisting of SEQ ID NO: 247, 248, 249, 250, 251, 252, 253, 254, 255, 256, 257, 258, 259, 260, 261, 262, 263, 264, 265, and/or 266, or naturally occurring variants thereof (e.g. sequences encoding a mammalian FUBP1).

In some embodiments, the target nucleic acid is selected from the group consisting of SEQ ID NO: 247, 251 and/or 255, or naturally occurring variants thereof (e.g. sequences encoding a mammalian FUBP1).

In some embodiments, the target nucleic acid is selected from the group consisting of SEQ ID NO: 247 and 251, or naturally occurring variants thereof (e.g. sequences encoding a mammalian FUBP1).

In some embodiments, the target nucleic acid is selected from the group consisting of SEQ ID NO: 247, to 254, or naturally occurring variants thereof (e.g. sequences encoding a mammalian FUBP1).

In some embodiments the target nucleic acid is a RNA or DNA which encodes mammalian FUBP1 protein, such as human FUBP1, e.g. the human FUBP1 mRNA sequence, such as that disclosed as SEQ ID NO 247. Further information on exemplary target nucleic acids is provided in tables 5 and 6.

TABLE 5
Genome and assembly information for FUBP1 across species.

| | | | Genomic coordinates | | | ensembl |
|---|---|---|---|---|---|---|
| Species | Chr. | Strand | Start | End | Assembly | gene_id |
| Human | 1 | Rv | 77944055 | 77979110 | GRCh38.p10 | ENSG00000162613 |
| Cyno | 1 | Fwd | 149243675 | 149283374 | Macaca_fascicularis_5.0 | ENSMFAG00000031825 |
| monkey | | | | | | |
| Mouse | 3 | Fwd | 152210422 | 152236826 | GRCm38.p5 | ENSMUSG00000028034 |

Fwd = forward strand. Rv = reverse strand. The genome coordinates provide the pre-mRNA sequence (genomic sequence).

If employing the nucleic acid molecule for use in the invention in research or diagnostics the target nucleic acid may be a cDNA or a synthetic nucleic acid derived from DNA or RNA.

For in vivo or in vitro application, the therapeutic nucleic acid molecule is typically capable of inhibiting the expression of the FUBP1 target nucleic acid in a cell which is expressing the FUBP1 target nucleic acid. The contiguous sequence of nucleobases of the nucleic acid molecule is typically complementary to a conserved region of the FUBP1 target nucleic acid, as measured across the length of the oligonucleotide, optionally with the exception of one or two mismatches, and optionally excluding nucleotide based linker regions which may link the oligonucleotide to an optional functional group such as a conjugate, or other non-complementary terminal nucleotides.

The target nucleic acid may be a messenger RNA, such as a pre-mRNA which encodes mammalian FUBP1 protein, such as human FUBP1, e.g. the human FUBP1 pre-mRNA sequence, such as that disclosed as SEQ ID NO: 247, the cynomolgus monkey FUBP1 pre-mRNA sequence, such as that disclosed as SEQ ID NO: 251, or the mouse FUBP1 pre-mRNA sequence, such as that disclosed as SEQ ID NO: 255, or a mature FUBP1 mRNA, such as a human mature mRNA disclosed as SEQ ID NO: 248, 249 and 250. SEQ ID NOs: 247-266 are DNA sequences—it will be understood that target RNA sequences have uracil (U) bases in place of the thymidine bases (T).

Further information on exemplary target nucleic acids is provided in table 6.

TABLE 6
Sequence details for FUBP1 across species.

| Species | RNA type | Length (nt) | SEQ ID NO |
|---|---|---|---|
| Human | Pre-mRNA | 305056 | 247 |
| Human | Mature mRNA | 696 | 248 |
| Human | Mature mRNA | 1968 | 249 |
| Human | Mature mRNA | 1935 | 250 |
| Cyno monkey | Pre-mRNA | 39750 | 251 |
| Cyno monkey | Mature mRNA | 1968 | 252 |
| Cyno monkey | Mature mRNA | 6825 | 253 |
| Cyno monkey | Mature mRNA | 1959 | 254 |
| Mouse | Pre-mRNA | 26405 | 255 |
| Mouse | Mature mRNA | 4525 | 256 |
| Mouse | Mature mRNA | 800 | 257 |
| Mouse | Mature mRNA | 2526 | 258 |
| Mouse | Mature mRNA | 809 | 259 |
| Mouse | Mature mRNA | 1040 | 260 |
| Mouse | Mature mRNA | 796 | 261 |
| Mouse | Mature mRNA | 585 | 262 |
| Mouse | Mature mRNA | 2374 | 263 |
| Mouse | Mature mRNA | 3163 | 264 |
| Mouse | Mature mRNA | 6523 | 265 |
| Mouse | Mature mRNA | 2552 | 266 |

Note SEQ ID NO 251 comprises regions of multiple NNNNs, where the sequencing has been unable to accurately refine the sequence, and a degenerate sequence is therefore included. For the avoidance of doubt the compounds of the combination of the invention are complementary to the actual target sequence and are not therefore degenerate compounds.

### Target Sequence

The term “target sequence” as used herein refers to a sequence of nucleotides present in the target nucleic acid which comprises the nucleobase sequence which is complementary to the oligonucleotide for use in the invention. In some embodiments, the target sequence consists of a region on the target nucleic acid with a nucleobase sequence that is complementary to the contiguous nucleotide sequence of the oligonucleotide for use in the invention. This region of the target nucleic acid may interchangeably be referred to as the target nucleotide sequence, target sequence or target region. In some embodiments the target sequence is longer than the complementary sequence of a single oligonucleotide, and may, for example represent a preferred region of the target nucleic acid which may be targeted by several oligonucleotides.

### RTEL1 Target Sequence

In some embodiments the target sequence is a sequence selected from the group consisting of a human RTEL1 mRNA exon, such as a RTEL1 human mRNA exon selected from the list in table 1 above.

In some embodiments the target sequence is a sequence selected from the group consisting of a human RTEL1 mRNA intron, such as a RTEL1 human mRNA intron selected from the list in table 1 above.

The oligonucleotide for use in the invention comprises a contiguous nucleotide sequence which is complementary to or hybridizes to the target nucleic acid, such as a target sequence described herein.

The target sequence to which the oligonucleotide is complementary or hybridizes to generally comprises a contiguous nucleobases sequence of at least 10 nucleotides. The contiguous nucleotide sequence is between 10 to 35 nucleotides, such as 12 to 30, such as 14 to 20, such as 16 to 20 contiguous nucleotides. In one embodiment of the invention the target sequence is selected from the group consisting of SEQ ID NO: 3-26 as shown in table 7.

TABLE 7
Target sequences on human RTEL1 premRNA (SEQ ID NO: 1)

| SEQ | | Start on | End on |
|---|---|---|---|
| ID NO | Target Sequence | SEQ ID 1 | SEQ ID 1 |
| 3 | gaccactgtccttccatg | 8294 | 8311 |
| 4 | ttcagagattcaagttataataaagctcttcttatattgaggggga | 8677 | 8722 |
| 5 | gagattcaagttataataaag | 8681 | 8701 |
| 6 | aggaatagggttggtttt | 9377 | 9394 |
| 7 | ccttactacctgtcccg | 9667 | 9683 |
| 8 | acaattacttgttggatgcc | 9722 | 9741 |
| 9 | agcttctaacccaaccag | 10921 | 10938 |
| 10 | tataaacctaaatgtaaaagc | 11482 | 11502 |
| 11 | ttcaccaaaatttaaagctt | 11622 | 11641 |
| 12 | ctttgaccagagtatgtaaaattctc | 11752 | 11777 |
| 13 | tttgaccagagtatgtaaaatt | 11753 | 11774 |
| 14 | tttgaccagagtatgtaa | 11753 | 11770 |
| 15 | gaccagagtatgtaaaatt | 11756 | 11774 |
| 16 | accagagtatgtaaaatt | 11757 | 11774 |
| 17 | aagacgtgttcaaagatt | 12868 | 12885 |
| 18 | ggacctactgttttttg | 13234 | 13250 |
| 19 | ggacctactgttttattcc | 13550 | 13568 |
| 20 | gtcccttctcttcctcctgtag | 14725 | 14746 |
| 21 | cgtgatctttgacgaagct | 14785 | 14803 |
| 22 | cgcaaacctttctgga | 14874 | 14889 |
| 23 | agcctgtgtgtggagtatgagca | 33025 | 33047 |
| 24 | cgtttccgtgttggtctggg | 34571 | 34590 |
| 25 | gactacaagggttccgatg | 35104 | 35122 |
| 26 | agtttgaggaggtctgtatc | 35370 | 35389 |

In some embodiments, the target sequence is SEQ ID NO 5.

In some embodiments, the target sequence is SEQ ID NO 13.

In some embodiments, the target sequence is SEQ ID NO 14.

In some embodiments, the target sequence is SEQ ID NO 15.

In some embodiments, the target sequence is SEQ ID NO 16.

SEQ ID NO: 5:
GAGATTCAAGTTATAATAAAG
SEQ ID NO 13:
TTTGACCAGAGTATGTAAAATT
SEQ ID NO: 14:
TTTGACCAGAGTATGTAA
SEQ ID NO: 15:
GACCAGAGTATGTAAAATT
SEQ ID NO 16:
ACCAGAGTATGTAAAATT

SEQ ID NOs: 3 to 26 are DNA sequences—it will be understood that target RNA sequences have uracil (U) bases in place of the thymidine bases (T).

The target sequences shown in SEQ ID NOs: 13 to 16 can be found in intron 8 of human RTEL1. The target sequence shown in SEQ ID No: 5 can be found in intron 7 of human RTEL1.

In some embodiments, the target sequence is the region from nucleotides 11753-11774 of SEQ ID NO: 1.

In some embodiments, the target sequence is the region from nucleotides 11757-11774 of SEQ ID NO: 1.

In some embodiments, the target sequence is the region from nucleotides 11756-11774 of SEQ ID NO: 1.

In some embodiments, the target sequence is the region from nucleotides 11753-11770 of SEQ ID NO: 1.

In some embodiments, the target sequence is the region from nucleotides 8681-8701 of SEQ ID NO: 1.

In some embodiments, the target sequence is selected from a region shown in Table 8A or 8B.

TABLE 8A
Regions of SEQ ID NO 1 which may be targeted using
an oligonucleotide for use in the invention

| | |
|---|---|
| Reg. | Position in SEQ ID NO 1 |

| A | from | to | Length |
|---|---|---|---|
| 1A | 1594 | 1623 | 30 |
| 2A | 1636 | 1685 | 50 |
| 3A | 1687 | 1708 | 22 |
| 4A | 1773 | 1794 | 22 |
| 5A | 1810 | 1824 | 15 |
| 6A | 1824 | 1870 | 47 |
| 7A | 2890 | 2907 | 18 |
| 8A | 2931 | 2952 | 22 |
| 9A | 2984 | 2999 | 16 |
| 10A | 3002 | 3021 | 20 |
| 11A | 3026 | 3081 | 56 |
| 12A | 3083 | 3100 | 18 |
| 13A | 3175 | 3202 | 28 |
| 14A | 3205 | 3228 | 24 |
| 15A | 3253 | 3270 | 18 |
| 16A | 3289 | 3320 | 32 |
| 17A | 3353 | 3368 | 16 |
| 18A | 3374 | 3388 | 15 |
| 19A | 3443 | 3472 | 30 |
| 20A | 3525 | 3547 | 23 |
| 21A | 3549 | 3581 | 33 |
| 22A | 3584 | 3612 | 29 |
| 23A | 3648 | 3675 | 28 |
| 24A | 3681 | 3708 | 28 |
| 25A | 3716 | 3731 | 16 |
| 26A | 3733 | 3755 | 23 |
| 27A | 3757 | 3775 | 19 |
| 28A | 3797 | 3811 | 15 |
| 29A | 3823 | 3844 | 22 |
| 30A | 3846 | 3881 | 36 |
| 31A | 3922 | 3955 | 34 |
| 32A | 3957 | 3975 | 19 |
| 33A | 4016 | 4036 | 21 |
| 34A | 4038 | 4053 | 16 |
| 35A | 4067 | 4098 | 32 |
| 36A | 4100 | 4150 | 51 |
| 37A | 4271 | 4290 | 20 |
| 38A | 4312 | 4330 | 19 |
| 39A | 4345 | 4362 | 18 |
| 40A | 4364 | 4379 | 16 |
| 41A | 4420 | 4448 | 29 |
| 42A | 4480 | 4512 | 33 |
| 43A | 4527 | 4552 | 26 |
| 44A | 4651 | 4674 | 24 |
| 45A | 4733 | 4784 | 52 |
| 46A | 4786 | 4811 | 26 |
| 47A | 4830 | 4848 | 19 |
| 48A | 4857 | 4874 | 18 |
| 49A | 4915 | 4937 | 23 |
| 50A | 4944 | 4966 | 23 |
| 51A | 4969 | 4983 | 15 |
| 52A | 4995 | 5015 | 21 |
| 53A | 5017 | 5033 | 17 |
| 54A | 5035 | 5075 | 41 |
| 55A | 5109 | 5136 | 28 |
| 56A | 5155 | 5173 | 19 |
| 57A | 5175 | 5191 | 17 |
| 58A | 5209 | 5225 | 17 |
| 59A | 5241 | 5261 | 21 |
| 60A | 5263 | 5287 | 25 |
| 61A | 5299 | 5346 | 48 |
| 62A | 5394 | 5409 | 16 |
| 63A | 5428 | 5447 | 20 |
| 64A | 5472 | 5514 | 43 |
| 65A | 5579 | 5606 | 28 |
| 66A | 5617 | 5632 | 16 |
| 67A | 5690 | 5711 | 22 |
| 68A | 5713 | 5754 | 42 |
| 69A | 5727 | 5741 | 15 |
| 70A | 5756 | 5770 | 15 |
| 71A | 5812 | 5854 | 43 |
| 72A | 5871 | 5886 | 16 |
| 73A | 5896 | 5932 | 37 |
| 74A | 5992 | 6009 | 18 |
| 75A | 6011 | 6038 | 28 |
| 76A | 6057 | 6072 | 16 |
| 77A | 6101 | 6122 | 22 |
| 78A | 6127 | 6165 | 39 |
| 79A | 6187 | 6203 | 17 |
| 80A | 6210 | 6227 | 18 |
| 81A | 6243 | 6261 | 19 |
| 82A | 6271 | 6299 | 29 |
| 83A | 6378 | 6393 | 16 |
| 84A | 6468 | 6496 | 29 |
| 85A | 6498 | 6526 | 29 |
| 86A | 6558 | 6574 | 17 |
| 87A | 6720 | 6737 | 18 |
| 88A | 6735 | 6749 | 15 |
| 89A | 6785 | 6825 | 41 |
| 90A | 6879 | 6894 | 16 |
| 91A | 6921 | 6959 | 39 |
| 92A | 6995 | 7060 | 66 |
| 93A | 7062 | 7084 | 23 |
| 94A | 7114 | 7146 | 33 |
| 95A | 7155 | 7186 | 32 |
| 96A | 7188 | 7203 | 16 |
| 97A | 7230 | 7267 | 38 |
| 98A | 7281 | 7299 | 19 |
| 99A | 7291 | 7316 | 26 |
| 100A | 7344 | 7369 | 26 |
| 101A | 7375 | 7391 | 17 |
| 102A | 7400 | 7414 | 15 |
| 103A | 7427 | 7441 | 15 |
| 104A | 7437 | 7453 | 17 |
| 105A | 7449 | 7463 | 15 |
| 106A | 7467 | 7481 | 15 |
| 107A | 7500 | 7518 | 19 |
| 108A | 7532 | 7546 | 15 |
| 109A | 7573 | 7587 | 15 |
| 110A | 7607 | 7621 | 15 |
| 111A | 7659 | 7685 | 27 |
| 112A | 7732 | 7767 | 36 |
| 113A | 7779 | 7793 | 15 |
| 114A | 7844 | 7882 | 39 |
| 115A | 7888 | 7910 | 23 |
| 116A | 7966 | 7980 | 15 |
| 117A | 8033 | 8047 | 15 |
| 118A | 8049 | 8063 | 15 |
| 119A | 8160 | 8178 | 19 |
| 120A | 8180 | 8195 | 16 |
| 121A | 8216 | 8237 | 22 |
| 122A | 8239 | 8341 | 103 |
| 123A | 8357 | 8373 | 17 |
| 124A | 8415 | 8430 | 16 |
| 125A | 8449 | 8465 | 17 |
| 126A | 8541 | 8560 | 20 |
| 127A | 8574 | 8596 | 23 |
| 128A | 8677 | 8703 | 27 |
| 129A | 8705 | 8722 | 18 |
| 130A | 8748 | 8763 | 16 |
| 131A | 8792 | 8807 | 16 |
| 132A | 8796 | 8811 | 16 |
| 133A | 8799 | 8818 | 20 |
| 134A | 8807 | 8821 | 15 |
| 135A | 8814 | 8828 | 15 |
| 136A | 8837 | 8853 | 17 |
| 137A | 8837 | 8851 | 15 |
| 138A | 8841 | 8858 | 18 |
| 139A | 8884 | 8911 | 28 |
| 140A | 8918 | 8937 | 20 |
| 141A | 8918 | 8933 | 16 |
| 142A | 8969 | 9005 | 37 |
| 143A | 8969 | 8999 | 31 |
| 144A | 8969 | 9000 | 32 |
| 145A | 8971 | 8989 | 19 |
| 146A | 8974 | 9000 | 27 |
| 147A | 8982 | 8999 | 18 |
| 148A | 9024 | 9042 | 19 |
| 149A | 9026 | 9042 | 17 |
| 150A | 9026 | 9040 | 15 |
| 151A | 9026 | 9041 | 16 |
| 152A | 9027 | 9043 | 17 |
| 153A | 9027 | 9041 | 15 |
| 154A | 9027 | 9042 | 16 |
| 155A | 9028 | 9044 | 17 |
| 156A | 9028 | 9042 | 15 |
| 157A | 9028 | 9043 | 16 |
| 158A | 9029 | 9045 | 17 |
| 159A | 9029 | 9043 | 15 |
| 160A | 9029 | 9044 | 16 |
| 161A | 9030 | 9046 | 17 |
| 162A | 9030 | 9044 | 15 |
| 163A | 9030 | 9045 | 16 |
| 164A | 9031 | 9047 | 17 |
| 165A | 9031 | 9045 | 15 |
| 166A | 9031 | 9046 | 16 |
| 167A | 9032 | 9048 | 17 |
| 168A | 9032 | 9046 | 15 |
| 169A | 9032 | 9047 | 16 |
| 170A | 9033 | 9047 | 15 |
| 171A | 9033 | 9048 | 16 |
| 172A | 9034 | 9048 | 15 |
| 173A | 9038 | 9052 | 15 |
| 174A | 9046 | 9064 | 19 |
| 175A | 9069 | 9090 | 22 |
| 176A | 9074 | 9089 | 16 |
| 177A | 9078 | 9093 | 16 |
| 178A | 9095 | 9110 | 16 |
| 179A | 9101 | 9124 | 24 |
| 180A | 9126 | 9161 | 36 |
| 181A | 9135 | 9155 | 21 |
| 182A | 9147 | 9162 | 16 |
| 183A | 9163 | 9186 | 24 |
| 184A | 9203 | 9222 | 20 |
| 185A | 9210 | 9253 | 44 |
| 186A | 9210 | 9230 | 21 |
| 187A | 9223 | 9254 | 32 |
| 188A | 9241 | 9256 | 16 |
| 189A | 9258 | 9272 | 15 |
| 190A | 9266 | 9303 | 38 |
| 191A | 9291 | 9308 | 18 |
| 192A | 9311 | 9329 | 19 |
| 193A | 9370 | 9394 | 25 |
| 194A | 9406 | 9420 | 15 |
| 195A | 9569 | 9591 | 23 |
| 196A | 9653 | 9708 | 56 |
| 197A | 9712 | 9758 | 47 |
| 198A | 9771 | 9788 | 18 |
| 199A | 9812 | 9829 | 18 |
| 200A | 9844 | 9862 | 19 |
| 201A | 9872 | 9917 | 46 |
| 202A | 9958 | 9983 | 26 |
| 203A | 9985 | 10002 | 18 |
| 204A | 10017 | 10054 | 38 |
| 205A | 10113 | 10132 | 20 |
| 206A | 10113 | 10130 | 18 |
| 207A | 10120 | 10137 | 18 |
| 208A | 10183 | 10204 | 22 |
| 209A | 10185 | 10204 | 20 |
| 210A | 10185 | 10202 | 18 |
| 211A | 10192 | 10209 | 18 |
| 212A | 10192 | 10210 | 19 |
| 213A | 10231 | 10251 | 21 |
| 214A | 10236 | 10251 | 16 |
| 215A | 10320 | 10337 | 18 |
| 216A | 10338 | 10353 | 16 |
| 217A | 10397 | 10415 | 19 |
| 218A | 10563 | 10584 | 22 |
| 219A | 10591 | 10607 | 17 |
| 220A | 10703 | 10723 | 21 |
| 221A | 10766 | 10784 | 19 |
| 222A | 10805 | 10822 | 18 |
| 223A | 10844 | 10870 | 27 |
| 224A | 10873 | 10893 | 21 |
| 225A | 10895 | 10913 | 19 |
| 226A | 10915 | 10942 | 28 |
| 227A | 10961 | 10975 | 15 |
| 228A | 10983 | 10999 | 17 |
| 229A | 11001 | 11015 | 15 |
| 230A | 11021 | 11035 | 15 |
| 231A | 11033 | 11059 | 27 |
| 232A | 11061 | 11082 | 22 |
| 233A | 11084 | 11104 | 21 |
| 234A | 11124 | 11154 | 31 |
| 235A | 11156 | 11170 | 15 |
| 236A | 11175 | 11192 | 18 |
| 237A | 11227 | 11260 | 34 |
| 238A | 11239 | 11254 | 16 |
| 239A | 11274 | 11302 | 29 |
| 240A | 11290 | 11305 | 16 |
| 241A | 11299 | 11317 | 19 |
| 242A | 11305 | 11329 | 25 |
| 243A | 11344 | 11361 | 18 |
| 244A | 11372 | 11400 | 29 |
| 245A | 11402 | 11416 | 15 |
| 246A | 11418 | 11445 | 28 |
| 247A | 11457 | 11471 | 15 |
| 248A | 11482 | 11511 | 30 |
| 249A | 11550 | 11566 | 17 |
| 250A | 11622 | 11645 | 24 |
| 251A | 11722 | 11737 | 16 |
| 252A | 11745 | 11777 | 33 |
| 253A | 11824 | 11844 | 21 |
| 254A | 11824 | 11840 | 17 |
| 255A | 12622 | 12638 | 17 |
| 256A | 12673 | 12691 | 19 |
| 257A | 12693 | 12724 | 32 |
| 258A | 12747 | 12763 | 17 |
| 259A | 12783 | 12806 | 24 |
| 260A | 12818 | 12837 | 20 |
| 261A | 12856 | 12885 | 30 |
| 262A | 12890 | 12912 | 23 |
| 263A | 12914 | 12945 | 32 |
| 264A | 12984 | 13016 | 33 |
| 265A | 13001 | 13016 | 16 |
| 266A | 13004 | 13022 | 19 |
| 267A | 13004 | 13021 | 18 |
| 268A | 13014 | 13034 | 21 |
| 269A | 13166 | 13191 | 26 |
| 270A | 13228 | 13251 | 24 |
| 271A | 13283 | 13319 | 37 |
| 272A | 13295 | 13310 | 16 |
| 273A | 13317 | 13332 | 16 |
| 274A | 13354 | 13381 | 28 |
| 275A | 13383 | 13430 | 48 |
| 276A | 13446 | 13468 | 23 |
| 277A | 13449 | 13468 | 20 |
| 278A | 13471 | 13487 | 17 |
| 279A | 13500 | 13518 | 19 |
| 280A | 13547 | 13568 | 22 |
| 281A | 13631 | 13650 | 20 |
| 282A | 13663 | 13679 | 17 |
| 283A | 13680 | 13694 | 15 |
| 284A | 13744 | 13764 | 21 |
| 285A | 13766 | 13803 | 38 |
| 286A | 13768 | 13803 | 36 |
| 287A | 13777 | 13797 | 21 |
| 288A | 13789 | 13804 | 16 |
| 289A | 13804 | 13827 | 24 |
| 290A | 13823 | 13844 | 22 |
| 291A | 13840 | 13854 | 15 |
| 292A | 13840 | 13855 | 16 |
| 293A | 13841 | 13855 | 15 |
| 294A | 13851 | 13874 | 24 |
| 295A | 13851 | 13873 | 23 |
| 296A | 13853 | 13871 | 19 |
| 297A | 13855 | 13874 | 20 |
| 298A | 13862 | 13882 | 21 |
| 299A | 13890 | 13905 | 16 |
| 300A | 13897 | 13927 | 31 |
| 301A | 13926 | 13940 | 15 |
| 302A | 13957 | 13971 | 15 |
| 303A | 13966 | 13980 | 15 |
| 304A | 13995 | 14025 | 31 |
| 305A | 14027 | 14048 | 22 |
| 306A | 14048 | 14067 | 20 |
| 307A | 14084 | 14098 | 15 |
| 308A | 14118 | 14133 | 16 |
| 309A | 14154 | 14171 | 18 |
| 310A | 14173 | 14210 | 38 |
| 311A | 14198 | 14218 | 21 |
| 312A | 14200 | 14218 | 19 |
| 313A | 14237 | 14265 | 29 |
| 314A | 14242 | 14265 | 24 |
| 315A | 14242 | 14264 | 23 |
| 316A | 14244 | 14262 | 19 |
| 317A | 14246 | 14265 | 20 |
| 318A | 14253 | 14271 | 19 |
| 319A | 14273 | 14293 | 21 |
| 320A | 14290 | 14304 | 15 |
| 321A | 14295 | 14320 | 26 |
| 322A | 14308 | 14352 | 45 |
| 323A | 14323 | 14352 | 30 |
| 324A | 14326 | 14352 | 27 |
| 325A | 14334 | 14351 | 18 |
| 326A | 14340 | 14364 | 25 |
| 327A | 14340 | 14359 | 20 |
| 328A | 14348 | 14362 | 15 |
| 329A | 14374 | 14406 | 33 |
| 330A | 14416 | 14446 | 31 |
| 331A | 14462 | 14489 | 28 |
| 332A | 14505 | 14521 | 17 |
| 333A | 14523 | 14541 | 19 |
| 334A | 14577 | 14598 | 22 |
| 335A | 14725 | 14762 | 38 |
| 336A | 14764 | 14781 | 18 |
| 337A | 14783 | 14808 | 26 |
| 338A | 14874 | 14905 | 32 |
| 339A | 14974 | 15030 | 57 |
| 340A | 15032 | 15059 | 28 |
| 341A | 15084 | 15098 | 15 |
| 342A | 15087 | 15106 | 20 |
| 343A | 15108 | 15126 | 19 |
| 344A | 15147 | 15180 | 34 |
| 345A | 15183 | 15202 | 20 |
| 346A | 15230 | 15247 | 18 |
| 347A | 15255 | 15270 | 16 |
| 348A | 15272 | 15298 | 27 |
| 349A | 15288 | 15312 | 25 |
| 350A | 15319 | 15349 | 31 |
| 351A | 15359 | 15373 | 15 |
| 352A | 15370 | 15385 | 16 |
| 353A | 15382 | 15400 | 19 |
| 354A | 15388 | 15408 | 21 |
| 355A | 15410 | 15435 | 26 |
| 356A | 15435 | 15452 | 18 |
| 357A | 15456 | 15498 | 43 |
| 358A | 15459 | 15474 | 16 |
| 359A | 15479 | 15498 | 20 |
| 360A | 15486 | 15502 | 17 |
| 361A | 15528 | 15543 | 16 |
| 362A | 15543 | 15561 | 19 |
| 363A | 15572 | 15591 | 20 |
| 364A | 15623 | 15642 | 20 |
| 365A | 15646 | 15660 | 15 |
| 366A | 15662 | 15690 | 29 |
| 367A | 15702 | 15740 | 39 |
| 368A | 15740 | 15754 | 15 |
| 369A | 15743 | 15773 | 31 |
| 370A | 15746 | 15761 | 16 |
| 371A | 15764 | 15789 | 26 |
| 372A | 15777 | 15803 | 27 |
| 373A | 15791 | 15816 | 26 |
| 374A | 15832 | 15848 | 17 |
| 375A | 15855 | 15873 | 19 |
| 376A | 15870 | 15890 | 21 |
| 377A | 15878 | 15908 | 31 |
| 378A | 15880 | 15898 | 19 |
| 379A | 15891 | 15908 | 18 |
| 380A | 15896 | 15916 | 21 |
| 381A | 15911 | 15929 | 19 |
| 382A | 15911 | 15930 | 20 |
| 383A | 15947 | 15963 | 17 |
| 384A | 16023 | 16056 | 34 |
| 385A | 16068 | 16091 | 24 |
| 386A | 16083 | 16097 | 15 |
| 387A | 16129 | 16150 | 22 |
| 388A | 16170 | 16229 | 60 |
| 389A | 16245 | 16265 | 21 |
| 390A | 16269 | 16300 | 32 |
| 391A | 16308 | 16334 | 27 |
| 392A | 16336 | 16356 | 21 |
| 393A | 16336 | 16358 | 23 |
| 394A | 16360 | 16391 | 32 |
| 395A | 16360 | 16397 | 38 |
| 396A | 16425 | 16465 | 41 |
| 397A | 16472 | 16493 | 22 |
| 398A | 16498 | 16515 | 18 |
| 399A | 16545 | 16562 | 18 |
| 400A | 16564 | 16586 | 23 |
| 401A | 16588 | 16613 | 26 |
| 402A | 16615 | 16639 | 25 |
| 403A | 16651 | 16667 | 17 |
| 404A | 16669 | 16695 | 27 |
| 405A | 16696 | 16716 | 21 |
| 406A | 16704 | 16718 | 15 |
| 407A | 16732 | 16760 | 29 |
| 408A | 16737 | 16760 | 24 |
| 409A | 16849 | 16865 | 17 |
| 410A | 16853 | 16868 | 16 |
| 411A | 16853 | 16867 | 15 |
| 412A | 16882 | 16897 | 16 |
| 413A | 16885 | 16902 | 18 |
| 414A | 16914 | 16938 | 25 |
| 415A | 16942 | 16956 | 15 |
| 416A | 16990 | 17004 | 15 |
| 417A | 17016 | 17042 | 27 |
| 418A | 17097 | 17115 | 19 |
| 419A | 17105 | 17119 | 15 |
| 420A | 17105 | 17126 | 22 |
| 421A | 17114 | 17128 | 15 |
| 422A | 17133 | 17158 | 26 |
| 423A | 17160 | 17174 | 15 |
| 424A | 17162 | 17178 | 17 |
| 425A | 17166 | 17183 | 18 |
| 426A | 17178 | 17199 | 22 |
| 427A | 17187 | 17201 | 15 |
| 428A | 17203 | 17223 | 21 |
| 429A | 17213 | 17230 | 18 |
| 430A | 17213 | 17235 | 23 |
| 431A | 17237 | 17259 | 23 |
| 432A | 17249 | 17279 | 31 |
| 433A | 17267 | 17285 | 19 |
| 434A | 17273 | 17288 | 16 |
| 435A | 17297 | 17315 | 19 |
| 436A | 17300 | 17315 | 16 |
| 437A | 17302 | 17317 | 16 |
| 438A | 17303 | 17324 | 22 |
| 439A | 17312 | 17330 | 19 |
| 440A | 17346 | 17375 | 30 |
| 441A | 17349 | 17375 | 27 |
| 442A | 17357 | 17374 | 18 |
| 443A | 17363 | 17382 | 20 |
| 444A | 17371 | 17385 | 15 |
| 445A | 17420 | 17447 | 28 |
| 446A | 17524 | 17551 | 28 |
| 447A | 17562 | 17580 | 19 |
| 448A | 17622 | 17636 | 15 |
| 449A | 17702 | 17734 | 33 |
| 450A | 17730 | 17745 | 16 |
| 451A | 17733 | 17755 | 23 |
| 452A | 17743 | 17758 | 16 |
| 453A | 17810 | 17824 | 15 |
| 454A | 17900 | 17940 | 41 |
| 455A | 17942 | 17968 | 27 |
| 456A | 17988 | 18002 | 15 |
| 457A | 18007 | 18024 | 18 |
| 458A | 18026 | 18042 | 17 |
| 459A | 18044 | 18059 | 16 |
| 460A | 18126 | 18159 | 34 |
| 461A | 18179 | 18205 | 27 |
| 462A | 18237 | 18253 | 17 |
| 463A | 18272 | 18290 | 19 |
| 464A | 18299 | 18314 | 16 |
| 465A | 18328 | 18344 | 17 |
| 466A | 18329 | 18344 | 16 |
| 467A | 18347 | 18361 | 15 |
| 468A | 18380 | 18402 | 23 |
| 469A | 18385 | 18399 | 15 |
| 470A | 18406 | 18421 | 16 |
| 471A | 18446 | 18473 | 28 |
| 472A | 18527 | 18543 | 17 |
| 473A | 18554 | 18569 | 16 |
| 474A | 18631 | 18645 | 15 |
| 475A | 18673 | 18693 | 21 |
| 476A | 18746 | 18765 | 20 |
| 477A | 18797 | 18824 | 28 |
| 478A | 18842 | 18860 | 19 |
| 479A | 18872 | 18892 | 21 |
| 480A | 18901 | 18915 | 15 |
| 481A | 18901 | 18940 | 40 |
| 482A | 18942 | 18976 | 35 |
| 483A | 18951 | 18976 | 26 |
| 484A | 18971 | 18994 | 24 |
| 485A | 18998 | 19016 | 19 |
| 486A | 19020 | 19039 | 20 |
| 487A | 19027 | 19043 | 17 |
| 488A | 19027 | 19050 | 24 |
| 489A | 19088 | 19102 | 15 |
| 490A | 19109 | 19129 | 21 |
| 491A | 19128 | 19145 | 18 |
| 492A | 19240 | 19258 | 19 |
| 493A | 19280 | 19366 | 87 |
| 494A | 19372 | 19387 | 16 |
| 495A | 19422 | 19444 | 23 |
| 496A | 19446 | 19462 | 17 |
| 497A | 19489 | 19506 | 18 |
| 498A | 19546 | 19571 | 26 |
| 499A | 19597 | 19615 | 19 |
| 500A | 19624 | 19648 | 25 |
| 501A | 19680 | 19695 | 16 |
| 502A | 19713 | 19727 | 15 |
| 503A | 19775 | 19792 | 18 |
| 504A | 19789 | 19803 | 15 |
| 505A | 19811 | 19825 | 15 |
| 506A | 19838 | 19862 | 25 |
| 507A | 20241 | 20257 | 17 |
| 508A | 20259 | 20290 | 32 |
| 509A | 20309 | 20381 | 73 |
| 510A | 20404 | 20419 | 16 |
| 511A | 20470 | 20492 | 23 |
| 512A | 20495 | 20557 | 63 |
| 513A | 20593 | 20609 | 17 |
| 514A | 20626 | 20646 | 21 |
| 515A | 20648 | 20669 | 22 |
| 516A | 20683 | 20699 | 17 |
| 517A | 20718 | 20735 | 18 |
| 518A | 20749 | 20765 | 17 |
| 519A | 20751 | 20765 | 15 |
| 520A | 20769 | 20785 | 17 |
| 521A | 20773 | 20791 | 19 |
| 522A | 20777 | 20798 | 22 |
| 523A | 20779 | 20798 | 20 |
| 524A | 20779 | 20797 | 19 |
| 525A | 20798 | 20819 | 22 |
| 526A | 20800 | 20819 | 20 |
| 527A | 20800 | 20818 | 19 |
| 528A | 20819 | 20840 | 22 |
| 529A | 20819 | 20853 | 35 |
| 530A | 20821 | 20840 | 20 |
| 531A | 20821 | 20853 | 33 |
| 532A | 20821 | 20839 | 19 |
| 533A | 20833 | 20851 | 19 |
| 534A | 20833 | 20855 | 23 |
| 535A | 20841 | 20864 | 24 |
| 536A | 20855 | 20869 | 15 |
| 537A | 20866 | 20895 | 30 |
| 538A | 20881 | 20902 | 22 |
| 539A | 20881 | 20915 | 35 |
| 540A | 20883 | 20902 | 20 |
| 541A | 20883 | 20915 | 33 |
| 542A | 20883 | 20901 | 19 |
| 543A | 20895 | 20913 | 19 |
| 544A | 20895 | 20917 | 23 |
| 545A | 20903 | 20926 | 24 |
| 546A | 20917 | 20931 | 15 |
| 547A | 20928 | 20946 | 19 |
| 548A | 20937 | 20951 | 15 |
| 549A | 20955 | 20973 | 19 |
| 550A | 20975 | 20993 | 19 |
| 551A | 20975 | 20997 | 23 |
| 552A | 20983 | 21004 | 22 |
| 553A | 20983 | 21017 | 35 |
| 554A | 20985 | 21004 | 20 |
| 555A | 20985 | 21017 | 33 |
| 556A | 20985 | 21003 | 19 |
| 557A | 20997 | 21015 | 19 |
| 558A | 20997 | 21019 | 23 |
| 559A | 21005 | 21028 | 24 |
| 560A | 21019 | 21033 | 15 |
| 561A | 21030 | 21048 | 19 |
| 562A | 21030 | 21052 | 23 |
| 563A | 21057 | 21075 | 19 |
| 564A | 21057 | 21079 | 23 |
| 565A | 21067 | 21085 | 19 |
| 566A | 21088 | 21118 | 31 |
| 567A | 21127 | 21153 | 27 |
| 568A | 21155 | 21169 | 15 |
| 569A | 21155 | 21180 | 26 |
| 570A | 21205 | 21220 | 16 |
| 571A | 21222 | 21283 | 62 |
| 572A | 21347 | 21370 | 24 |
| 573A | 21431 | 21445 | 15 |
| 574A | 21463 | 21487 | 25 |
| 575A | 21489 | 21518 | 30 |
| 576A | 21520 | 21535 | 16 |
| 577A | 21551 | 21573 | 23 |
| 578A | 21574 | 21591 | 18 |
| 579A | 21595 | 21618 | 24 |
| 580A | 21622 | 21641 | 20 |
| 581A | 21664 | 21678 | 15 |
| 582A | 21758 | 21789 | 32 |
| 583A | 21799 | 21816 | 18 |
| 584A | 21820 | 21852 | 33 |
| 585A | 21865 | 21882 | 18 |
| 586A | 21890 | 21905 | 16 |
| 587A | 21917 | 21932 | 16 |
| 588A | 21956 | 21976 | 21 |
| 589A | 21975 | 21993 | 19 |
| 590A | 22007 | 22035 | 29 |
| 591A | 22014 | 22034 | 21 |
| 592A | 22036 | 22051 | 16 |
| 593A | 22036 | 22068 | 33 |
| 594A | 22070 | 22132 | 63 |
| 595A | 22174 | 22203 | 30 |
| 596A | 22205 | 22219 | 15 |
| 597A | 22229 | 22254 | 26 |
| 598A | 22276 | 22299 | 24 |
| 599A | 22309 | 22353 | 45 |
| 600A | 22359 | 22373 | 15 |
| 601A | 22385 | 22403 | 19 |
| 602A | 22443 | 22460 | 18 |
| 603A | 22462 | 22490 | 29 |
| 604A | 22499 | 22520 | 22 |
| 605A | 22601 | 22623 | 23 |
| 606A | 22646 | 22661 | 16 |
| 607A | 22663 | 22682 | 20 |
| 608A | 22713 | 22735 | 23 |
| 609A | 22737 | 22772 | 36 |
| 610A | 22793 | 22826 | 34 |
| 611A | 22851 | 22903 | 53 |
| 612A | 22905 | 22928 | 24 |
| 613A | 22934 | 22985 | 52 |
| 614A | 23071 | 23089 | 19 |
| 615A | 23094 | 23121 | 28 |
| 616A | 23174 | 23208 | 35 |
| 617A | 23249 | 23276 | 28 |
| 618A | 23279 | 23311 | 33 |
| 619A | 23313 | 23328 | 16 |
| 620A | 23450 | 23470 | 21 |
| 621A | 23488 | 23503 | 16 |
| 622A | 23511 | 23529 | 19 |
| 623A | 23555 | 23570 | 16 |
| 624A | 23575 | 23589 | 15 |
| 625A | 23597 | 23620 | 24 |
| 626A | 23632 | 23647 | 16 |
| 627A | 23672 | 23687 | 16 |
| 628A | 23737 | 23775 | 39 |
| 629A | 23746 | 23760 | 15 |
| 630A | 23833 | 23847 | 15 |
| 631A | 23872 | 23911 | 40 |
| 632A | 23919 | 23936 | 18 |
| 633A | 24050 | 24068 | 19 |
| 634A | 24083 | 24111 | 29 |
| 635A | 24111 | 24125 | 15 |
| 636A | 24131 | 24164 | 34 |
| 637A | 24167 | 24189 | 23 |
| 638A | 24204 | 24227 | 24 |
| 639A | 24236 | 24285 | 50 |
| 640A | 24438 | 24453 | 16 |
| 641A | 24499 | 24514 | 16 |
| 642A | 24560 | 24575 | 16 |
| 643A | 24621 | 24636 | 16 |
| 644A | 24682 | 24697 | 16 |
| 645A | 24717 | 24753 | 37 |
| 646A | 24842 | 24857 | 16 |
| 647A | 24902 | 24918 | 17 |
| 648A | 24932 | 24962 | 31 |
| 649A | 25018 | 25056 | 39 |
| 650A | 25160 | 25176 | 17 |
| 651A | 25219 | 25251 | 33 |
| 652A | 25259 | 25278 | 20 |
| 653A | 25332 | 25346 | 15 |
| 654A | 25363 | 25379 | 17 |
| 655A | 25367 | 25383 | 17 |
| 656A | 25405 | 25435 | 31 |
| 657A | 25405 | 25436 | 32 |
| 658A | 25407 | 25425 | 19 |
| 659A | 25410 | 25436 | 27 |
| 660A | 25418 | 25435 | 18 |
| 661A | 25475 | 25495 | 21 |
| 662A | 25502 | 25518 | 17 |
| 663A | 25559 | 25582 | 24 |
| 664A | 25596 | 25640 | 45 |
| 665A | 25671 | 25688 | 18 |
| 666A | 25796 | 25816 | 21 |
| 667A | 25818 | 25832 | 15 |
| 668A | 25834 | 25857 | 24 |
| 669A | 25867 | 25881 | 15 |
| 670A | 25928 | 25943 | 16 |
| 671A | 25986 | 26001 | 16 |
| 672A | 26014 | 26037 | 24 |
| 673A | 26187 | 26210 | 24 |
| 674A | 26212 | 26228 | 17 |
| 675A | 26268 | 26286 | 19 |
| 676A | 26300 | 26319 | 20 |
| 677A | 26359 | 26394 | 36 |
| 678A | 26396 | 26426 | 31 |
| 679A | 26465 | 26482 | 18 |
| 680A | 26505 | 26529 | 25 |
| 681A | 26547 | 26565 | 19 |
| 682A | 26576 | 26600 | 25 |
| 683A | 26588 | 26603 | 16 |
| 684A | 26588 | 26606 | 19 |
| 685A | 26609 | 26624 | 16 |
| 686A | 26615 | 26638 | 24 |
| 687A | 26615 | 26642 | 28 |
| 688A | 26632 | 26669 | 38 |
| 689A | 26649 | 26669 | 21 |
| 690A | 26658 | 26672 | 15 |
| 691A | 26695 | 26716 | 22 |
| 692A | 26706 | 26725 | 20 |
| 693A | 26713 | 26735 | 23 |
| 694A | 26715 | 26733 | 19 |
| 695A | 26737 | 26768 | 32 |
| 696A | 26755 | 26770 | 16 |
| 697A | 26756 | 26789 | 34 |
| 698A | 26759 | 26789 | 31 |
| 699A | 26787 | 26813 | 27 |
| 700A | 26795 | 26812 | 18 |
| 701A | 26815 | 26829 | 15 |
| 702A | 26861 | 26880 | 20 |
| 703A | 26862 | 26882 | 21 |
| 704A | 26865 | 26883 | 19 |
| 705A | 26868 | 26883 | 16 |
| 706A | 26870 | 26885 | 16 |
| 707A | 26871 | 26892 | 22 |
| 708A | 26880 | 26898 | 19 |
| 709A | 26889 | 26910 | 22 |
| 710A | 26908 | 26924 | 17 |
| 711A | 26917 | 26939 | 23 |
| 712A | 26948 | 26962 | 15 |
| 713A | 26955 | 26973 | 19 |
| 714A | 27097 | 27113 | 17 |
| 715A | 27101 | 27128 | 28 |
| 716A | 27112 | 27127 | 16 |
| 717A | 27116 | 27183 | 68 |
| 718A | 27133 | 27165 | 33 |
| 719A | 27188 | 27209 | 22 |
| 720A | 27218 | 27232 | 15 |
| 721A | 27218 | 27234 | 17 |
| 722A | 27235 | 27253 | 19 |
| 723A | 27237 | 27253 | 17 |
| 724A | 27237 | 27251 | 15 |
| 725A | 27237 | 27252 | 16 |
| 726A | 27238 | 27254 | 17 |
| 727A | 27238 | 27252 | 15 |
| 728A | 27238 | 27253 | 16 |
| 729A | 27239 | 27255 | 17 |
| 730A | 27239 | 27253 | 15 |
| 731A | 27239 | 27254 | 16 |
| 732A | 27240 | 27254 | 15 |
| 733A | 27240 | 27255 | 16 |
| 734A | 27241 | 27255 | 15 |
| 735A | 27269 | 27320 | 52 |
| 736A | 27281 | 27297 | 17 |
| 737A | 27286 | 27307 | 22 |
| 738A | 27340 | 27358 | 19 |
| 739A | 27360 | 27404 | 45 |
| 740A | 27411 | 27438 | 28 |
| 741A | 27458 | 27474 | 17 |
| 742A | 27531 | 27572 | 42 |
| 743A | 27575 | 27600 | 26 |
| 744A | 27602 | 27616 | 15 |
| 745A | 27618 | 27637 | 20 |
| 746A | 27670 | 27684 | 15 |
| 747A | 27707 | 27721 | 15 |
| 748A | 27723 | 27747 | 25 |
| 749A | 27772 | 27816 | 45 |
| 750A | 27772 | 27790 | 19 |
| 751A | 27829 | 27847 | 19 |
| 752A | 27850 | 27868 | 19 |
| 753A | 27870 | 27905 | 36 |
| 754A | 27927 | 27942 | 16 |
| 755A | 27963 | 27987 | 25 |
| 756A | 27989 | 28083 | 95 |
| 757A | 28085 | 28103 | 19 |
| 758A | 28120 | 28138 | 19 |
| 759A | 28167 | 28188 | 22 |
| 760A | 28190 | 28207 | 18 |
| 761A | 28209 | 28231 | 23 |
| 762A | 28234 | 28250 | 17 |
| 763A | 28260 | 28303 | 44 |
| 764A | 28427 | 28444 | 18 |
| 765A | 28446 | 28462 | 17 |
| 766A | 28464 | 28484 | 21 |
| 767A | 28503 | 28519 | 17 |
| 768A | 28521 | 28536 | 16 |
| 769A | 28538 | 28565 | 28 |
| 770A | 28595 | 28612 | 18 |
| 771A | 28694 | 28709 | 16 |
| 772A | 28701 | 28715 | 15 |
| 773A | 28715 | 28751 | 37 |
| 774A | 28801 | 28825 | 25 |
| 775A | 28832 | 28846 | 15 |
| 776A | 28846 | 28870 | 25 |
| 777A | 28878 | 28893 | 16 |
| 778A | 28895 | 28911 | 17 |
| 779A | 28938 | 28961 | 24 |
| 780A | 29010 | 29025 | 16 |
| 781A | 29057 | 29072 | 16 |
| 782A | 29119 | 29134 | 16 |
| 783A | 29179 | 29193 | 15 |
| 784A | 29235 | 29256 | 22 |
| 785A | 29330 | 29349 | 20 |
| 786A | 29367 | 29381 | 15 |
| 787A | 29530 | 29556 | 27 |
| 788A | 29587 | 29605 | 19 |
| 789A | 29652 | 29692 | 41 |
| 790A | 29695 | 29710 | 16 |
| 791A | 29722 | 29742 | 21 |
| 792A | 29743 | 29768 | 26 |
| 793A | 29770 | 29797 | 28 |
| 794A | 29818 | 29836 | 19 |
| 795A | 29838 | 29873 | 36 |
| 796A | 29875 | 29946 | 72 |
| 797A | 29948 | 29983 | 36 |
| 798A | 30028 | 30048 | 21 |
| 799A | 30046 | 30060 | 15 |
| 800A | 30051 | 30067 | 17 |
| 801A | 30069 | 30090 | 22 |
| 802A | 30093 | 30107 | 15 |
| 803A | 30116 | 30136 | 21 |
| 804A | 30138 | 30202 | 65 |
| 805A | 30220 | 30262 | 43 |
| 806A | 30303 | 30320 | 18 |
| 807A | 30349 | 30372 | 24 |
| 808A | 30387 | 30418 | 32 |
| 809A | 30417 | 30441 | 25 |
| 810A | 30476 | 30516 | 41 |
| 811A | 30524 | 30576 | 53 |
| 812A | 30602 | 30628 | 27 |
| 813A | 30658 | 30680 | 23 |
| 814A | 30682 | 30747 | 66 |
| 815A | 30749 | 30799 | 51 |
| 816A | 30801 | 30821 | 21 |
| 817A | 30823 | 30844 | 22 |
| 818A | 30908 | 30922 | 15 |
| 819A | 30924 | 30980 | 57 |
| 820A | 31027 | 31045 | 19 |
| 821A | 31047 | 31080 | 34 |
| 822A | 31086 | 31113 | 28 |
| 823A | 31128 | 31146 | 19 |
| 824A | 31150 | 31164 | 15 |
| 825A | 31166 | 31193 | 28 |
| 826A | 31229 | 31271 | 43 |
| 827A | 31276 | 31310 | 35 |
| 828A | 31312 | 31333 | 22 |
| 829A | 31400 | 31417 | 18 |
| 830A | 31419 | 31433 | 15 |
| 831A | 31456 | 31470 | 15 |
| 832A | 31517 | 31569 | 53 |
| 833A | 31578 | 31599 | 22 |
| 834A | 31661 | 31689 | 29 |
| 835A | 31706 | 31739 | 34 |
| 836A | 31741 | 31763 | 23 |
| 837A | 31765 | 31805 | 41 |
| 838A | 31807 | 31855 | 49 |
| 839A | 31819 | 31834 | 16 |
| 840A | 31851 | 31866 | 16 |
| 841A | 31857 | 31872 | 16 |
| 842A | 31938 | 31984 | 47 |
| 843A | 31986 | 32032 | 47 |
| 844A | 32034 | 32071 | 38 |
| 845A | 32082 | 32097 | 16 |
| 846A | 32124 | 32151 | 28 |
| 847A | 32197 | 32216 | 20 |
| 848A | 32233 | 32262 | 30 |
| 849A | 32264 | 32289 | 26 |
| 850A | 32306 | 32325 | 20 |
| 851A | 32357 | 32408 | 52 |
| 852A | 32410 | 32459 | 50 |
| 853A | 32474 | 32492 | 19 |
| 854A | 32494 | 32508 | 15 |
| 855A | 32527 | 32543 | 17 |
| 856A | 32545 | 32560 | 16 |
| 857A | 32570 | 32636 | 67 |
| 858A | 32697 | 32713 | 17 |
| 859A | 32744 | 32765 | 22 |
| 860A | 32801 | 32823 | 23 |
| 861A | 32865 | 32892 | 28 |
| 862A | 32944 | 32959 | 16 |
| 863A | 32962 | 32985 | 24 |
| 864A | 32998 | 33104 | 107 |
| 865A | 33126 | 33140 | 15 |
| 866A | 33142 | 33194 | 53 |
| 867A | 33213 | 33252 | 40 |
| 868A | 33277 | 33298 | 22 |
| 869A | 33318 | 33365 | 48 |
| 870A | 33375 | 33390 | 16 |
| 871A | 33402 | 33417 | 16 |
| 872A | 33419 | 33443 | 25 |
| 873A | 33456 | 33488 | 33 |
| 874A | 33509 | 33542 | 34 |
| 875A | 33562 | 33583 | 22 |
| 876A | 33607 | 33622 | 16 |
| 877A | 33655 | 33700 | 46 |
| 878A | 33704 | 33720 | 17 |
| 879A | 33735 | 33753 | 19 |
| 880A | 33755 | 33780 | 26 |
| 881A | 33806 | 33820 | 15 |
| 882A | 33829 | 33845 | 17 |
| 883A | 33916 | 33962 | 47 |
| 884A | 33964 | 33982 | 19 |
| 885A | 33989 | 34026 | 38 |
| 886A | 34028 | 34072 | 45 |
| 887A | 34089 | 34104 | 16 |
| 888A | 34113 | 34130 | 18 |
| 889A | 34141 | 34158 | 18 |
| 890A | 34281 | 34309 | 29 |
| 891A | 34377 | 34407 | 31 |
| 892A | 34423 | 34498 | 76 |
| 893A | 34507 | 34521 | 15 |
| 894A | 34524 | 34545 | 22 |
| 895A | 34552 | 34596 | 45 |
| 896A | 34688 | 34703 | 16 |
| 897A | 34742 | 34759 | 18 |
| 898A | 34770 | 34798 | 29 |
| 899A | 34860 | 34882 | 23 |
| 900A | 34919 | 34938 | 20 |
| 901A | 34950 | 34988 | 39 |
| 902A | 34990 | 35012 | 23 |
| 903A | 35022 | 35048 | 27 |
| 904A | 35063 | 35182 | 120 |
| 905A | 35184 | 35210 | 27 |
| 906A | 35222 | 35241 | 20 |
| 907A | 35245 | 35275 | 31 |
| 908A | 35277 | 35297 | 21 |
| 909A | 35319 | 35355 | 37 |
| 910A | 35367 | 35397 | 31 |
| 911A | 35433 | 35457 | 25 |
| 912A | 35461 | 35486 | 26 |
| 913A | 35490 | 35509 | 20 |
| 914A | 35546 | 35560 | 15 |
| 915A | 35573 | 35593 | 21 |
| 916A | 35597 | 35613 | 17 |
| 917A | 35968 | 35999 | 32 |
| 918A | 35997 | 36011 | 15 |
| 919A | 36037 | 36051 | 15 |
| 920A | 36097 | 36118 | 22 |
| 921A | 36117 | 36132 | 16 |
| 922A | 36278 | 36295 | 18 |
| 923A | 36350 | 36364 | 15 |
| 924A | 36366 | 36392 | 27 |
| 925A | 36433 | 36458 | 26 |
| 926A | 36460 | 36483 | 24 |
| 927A | 36530 | 36547 | 18 |
| 928A | 36549 | 36566 | 18 |
| 929A | 36600 | 36625 | 26 |
| 930A | 36627 | 36665 | 39 |
| 931A | 36759 | 36774 | 16 |
| 932A | 36765 | 36782 | 18 |
| 933A | 36815 | 36850 | 36 |
| 934A | 36873 | 36891 | 19 |
| 935A | 36894 | 36934 | 41 |
| 936A | 36969 | 36994 | 26 |
| 937A | 36996 | 37016 | 21 |
| 938A | 37023 | 37040 | 18 |
| 939A | 37093 | 37112 | 20 |
| 940A | 37118 | 37142 | 25 |
| 941A | 37144 | 37163 | 20 |
| 942A | 37242 | 37324 | 83 |
| 943A | 37352 | 37368 | 17 |
| 944A | 37370 | 37389 | 20 |
| 945A | 37391 | 37419 | 29 |
| 946A | 37421 | 37438 | 18 |
| 947A | 37444 | 37491 | 48 |
| 948A | 37511 | 37538 | 28 |
| 949A | 37567 | 37614 | 48 |
| 950A | 37636 | 37680 | 45 |
| 951A | 37723 | 37765 | 43 |
| 952A | 37773 | 37801 | 29 |
| 953A | 37803 | 37822 | 20 |
| 954A | 37824 | 37853 | 30 |
| 955A | 37855 | 37887 | 33 |
| 956A | 37889 | 37908 | 20 |
| 957A | 37920 | 37939 | 20 |
| 958A | 37988 | 38020 | 33 |
| 959A | 38022 | 38049 | 28 |

TABLE 8B
Regions of SEQ ID NO 1 which may be targeted using
an oligonucleotide for use in the invention

| | |
|---|---|
| Reg. | Position in SEQ ID NO 1 |

| B | from | to | Length |
|---|---|---|---|
| 1 | 8295 | 8312 | 17 |
| 2 | 8684 | 8704 | 20 |
| 3 | 9668 | 9684 | 16 |
| 4 | 9669 | 9684 | 15 |
| 5 | 9722 | 9741 | 19 |
| 6 | 9723 | 9741 | 18 |
| 7 | 9724 | 9742 | 18 |
| 8 | 10921 | 10937 | 16 |
| 9 | 11483 | 11503 | 20 |
| 10 | 11512 | 11531 | 19 |
| 11 | 11622 | 11641 | 19 |
| 12 | 11753 | 11773 | 20 |
| 13 | 11755 | 11772 | 17 |
| 14 | 11756 | 11776 | 20 |
| 15 | 11757 | 11776 | 19 |
| 16 | 11758 | 11778 | 20 |
| 17 | 12868 | 11885 | 17 |
| 18 | 13234 | 13252 | 18 |
| 19 | 13551 | 13569 | 18 |
| 20 | 14786 | 14804 | 18 |
| 21 | 18085 | 18101 | 16 |
| 22 | 22425 | 22441 | 16 |
| 23 | 33030 | 33048 | 18 |
| 24 | 35103 | 35123 | 20 |
| 25 | 35371 | 35390 | 19 |
| 26 | 35636 | 35655 | 19 |
| 27 | 35638 | 35654 | 16 |
| 28 | 36915 | 36931 | 16 |

### FUBP1 Target Sequence

In some embodiments the target sequence is a sequence selected from the group consisting of a human FUBP1 mRNA exon, such as a FUBP1 human mRNA exon selected from the group consisting of e1, e2, e3, e4, e5, e6, e7, e8, e9, e10, e11, e12, 13, e14, e15, e16, e17, e18, e19 and e20 (see for example table 4 above).

In one embodiment the target sequence is a sequence selected from the group consisting of one or more of human FUBP1 mRNA exons selected from the group consisting of exon 9, 10, 12, 14 and 20.

In some embodiments the target sequence is a sequence selected from the group consisting of a human FUBP1mRNA intron, such as a FUBP1 human mRNA intron selected from the group consisting of i1, i2, i3, i4, i5, i6, i7, i9, i10, i11, i12, 13, i14, i15, i16, i17, i18 and i19 (see for example table 4 above).

The nucleic acid molecule of the combination of the invention comprises a contiguous nucleotide sequence which is complementary to or hybridizes to a region on the target nucleic acid, such as a target sequence described herein.

In one embodiment, the target sequence is exon 14 of human FUBP1 mRNA (see Table 4 above).

In another embodiment, the target sequence is exon 20 of human FUBP1 mRNA (see Table 4 above).

The antisense oligonucleotide of the combination of the invention comprises a contiguous nucleotide sequence, which is complementary to or hybridizes to a region on the target nucleic acid, such as a target sequence described herein.

Provided herein below are target sequence regions, as defined by regions of the human FUBP1 pre-mRNA (using SEQ ID NO 247 as a reference) which may be targeted by the oligonucleotides of the combination of the invention.

The oligonucleotide of the combination of the invention comprises a contiguous nucleotide sequence, which is complementary to or hybridizes to the target nucleic acid, such as a sub-sequence of the target nucleic acid, such as a target sequence described herein. The target nucleic acid sequence to which the therapeutic nucleic acid molecule is complementary or hybridizes to generally comprises a stretch of contiguous nucleobases of at least 10 nucleotides. The contiguous nucleotide sequence (and therefore the target sequence) comprises at least 12 contiguous nucleotides, such as 12, 13, 14, 15, 16, 17, 18, 19, 20, 21, 22, 23, 24 nucleotides, such as from 14-20, such as from 14-18 contiguous nucleotides.

The inventors have identified particularly effective sequences of the FUBP1 target nucleic acid, which may be targeted by the oligonucleotide of the combination of the invention.

In some embodiments, the target sequence is SEQ ID NO: 267.

In some embodiments, the target sequence is SEQ ID NO: 268.

In some embodiments, the target sequence is SEQ ID NO: 269.

In some embodiments, the target sequence is SEQ ID NO: 270.

In some embodiment, the target sequence is SEQ ID NO: 347

SEQ ID NO: 267:
GTGAAACCATAAAAAGCATAAG
SEQ ID NO: 268:
AACCATAAAAAGCATAAG
SEQ ID NO: 269:
GTGAAACCATAAAAAGCATA
SEQ ID NO: 270:
GTAGAAATGAAAATTGGT
SEQ ID NO: 347:
GACTATGGTTATGGGG

SEQ ID NO: 267, 268 269, 270 and 347 are DNA sequences—it will be understood that target RNA sequences have uracil (U) bases in place of the thymidine bases (T).

The invention provides for an antisense oligonucleotide, which comprises a contiguous nucleotide sequence, which is complementary to, such as fully complementary to a region from nucleotides 16184 to 16205 of the human FUBP1 pre-mRNA (as illustrated in SEQ ID NO: 247).

The invention provides for an antisense oligonucleotide, which comprises a contiguous nucleotide sequence, which is complementary to, such as fully complementary to a region from nucleotides 16188 to 16205 of the human FUBP1 pre-mRNA (as illustrated in SEQ ID NO: 247).

The invention provides for an antisense oligonucleotide, which comprises a contiguous nucleotide sequence, which is complementary to, such as fully complementary to a region from nucleotides 16184 to 16203 of the human FUBP1 pre-mRNA (as illustrated in SEQ ID NO: 247).

Also, the invention provides for an antisense oligonucleotide, which comprises a contiguous nucleotide sequence, which is complementary to, such as fully complementary to a region from nucleotides 30536-30553 of the human FUBP1 pre-mRNA (as illustrated in SEQ ID NO: 247). Also, the invention provides for an antisense oligonucleotide, which comprises a contiguous nucleotide sequence, which is complementary to, such as fully complementary to a region from nucleotides 9141-9156 of the human FUBP1 pre-mRNA (as illustrated in SEQ ID NO: 247). In some embodiments, the antisense oligonucleotide or the contiguous nucleotide sequence is complementary to, such as fully complementary to a region from nucleotides 16184 to 16200 of SEQ ID NO: 247.

In some embodiments, the antisense oligonucleotide or the contiguous nucleotide sequence is complementary to, such as fully complementary to a region from nucleotides 16186 to 16203 of SEQ ID NO: 247.

In some embodiments, the antisense oligonucleotide or the contiguous nucleotide sequence is complementary to, such as fully complementary to a region from nucleotides 16189 to 16205 of SEQ ID NO: 247.

In some embodiments, the target sequence is the region from nucleotides 16184 to 16200 of SEQ ID NO: 247.

In some embodiments, the target sequence is the region from nucleotides 16186 to 16203 of SEQ ID NO: 247.

In some embodiments, the target sequence is the region from nucleotides 16188 to 16205 of SEQ ID NO: 247.

In some embodiments, the target sequence is the region from nucleotides 16189 to 16205 of SEQ ID NO: 247.

### The Target

The term “target” as used herein may refer to the mammalian protein RTEL1 (“Regulator of telomere elongation helicase 1), alternatively known as “KIAA1088” or “C20ORF41” or “Regulator of telomere length” or “Telomere length regulator” or “Chromosome 20 open reading frame 41”. The Homo sapiens RTEL1 gene is located at chromosome 20, 63,657,810 to 63,696,253, complement (Homo sapiens Updated Annotation, Release 109.20200228, GRCh38.p13). The RTEL1 protein is an ATP-dependent DNA helicase implicated in telomere-length regulation, DNA repair and the maintenance of genomic stability. The amino acid sequence of human RTEL1 is known in the art and can be assessed via UniProt, see UniProt entry Q9NZ71 for human RTEL1, hereby incorporated by reference.

The term “target” may also be used herein to refer the mammalian protein “Far Upstream Element-Binding Protein 1”, alternatively known as “FUBP1” or “FBP” or “FUBP” or “hDH V”. The Homo sapiens FUBP1 gene is located at chromosome 1, 77944055 . . . 77979435, complement (NC_000001.11, Gene ID 1462). The FUBP1 gene encodes a ssDNA binding protein that activates the far upstream element of c-myc and stimulates expression of c-myc in undifferentiated cells. Regulation of FUSE by FUBP occurs through single-strand binding of FUBP to the non-coding strand. The FUBP1 protein has ATP-dependent DNA helicase activity. The amino acid sequence of human FUBP1 is known in the art and can be assessed via UniProt, see e.g. UniProt entry Q96AE4 for human FUBP1, hereby incorporated by reference.

### Therapeutic Effective Amount

The term “therapeutically effective amount” denotes an amount of a compound the pharmaceutical combination of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

### Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic. Prophylactic can be understood as preventing an HBV infection from turning into a chronic HBV infection or the prevention of severe liver diseases such as liver cirrhosis and hepatocellular carcinoma caused by a chronic HBV infection.

### DETAILED DESCRIPTION OF THE INVENTION

HBV cccDNA in infected hepatocytes is responsible for persistent chronic infection and reactivation, being the template for all viral subgenomic transcripts and pre-genomic RNA (pgRNA) to ensure both newly synthesized viral progeny and cccDNA pool replenishment via intracellular nucleocapsid recycling. In the context of the present invention it was for the first time shown that RTEL1 is associated with cccDNA stability. This knowledge allows for the opportunity to destabilize cccDNA in HBV infected subjects which in turn opens the opportunity for a complete cure of chronically infected HBV patients.

Overexpression of and mutations in FUBP1 has been known to be associated with cancers for many years. In particular, strong overexpression of FUBP1 in human hepatocellular carcinoma (HCC) supports tumour growth and correlates with poor patient prognosis. HBV cccDNA in infected hepatocytes is responsible for persistent chronic infection and reactivation, being the template for all viral subgenomic transcripts and pre-genomic RNA (pgRNA) to ensure both newly synthesized viral progeny and cccDNA pool replenishment via intracellular nucleocapsid recycling. In the context of the present invention it was for the first time shown that FUBP1 is associated with cccDNA stability. This knowledge allows for the opportunity to destabilize cccDNA in HBV infected subjects which in turn opens the opportunity for a complete cure of chronically infected HBV patients. The role of FUBP1 in HCC and cccDNA stability is expected to be different and independent of each other.

The present invention relates to a combination of two categories of compounds i) an inhibitor of RTEL1 and ii) an inhibitor of FUBP1, or a pharmaceutically acceptable salts thereof. Suitably, each compound is provided in a pharmaceutically acceptable diluent, solvent, carrier, salt and/or adjuvant.

Suitably, the combination according to the invention is for use in treatment of Hepatitis B virus infections and/or cancer, in particular treatment of patients with a chronic HBV infection.

In an embodiment, the combination of the invention is a composition, a pharmaceutical composition, or a kit comprising of compounds i) an inhibitor of RTEL1 and ii) an inhibitor of FUBP1, or a pharmaceutically acceptable salt thereof. Suitably, each compound is provided in a pharmaceutically acceptable diluent, solvent, carrier, salt and/or adjuvant.

The invention also relates to a method for treating or preventing a disease comprising administering a combination according to the present invention.

The invention also relates to the use of a combination according to the present invention, for the preparation of a medicament.

The invention also relates to an in vivo or in vitro method for modulating RTEL1 and FUBP1 expression in a target cell which is expressing RTEL1 and FUBP1, said method comprising administering the combination according to the present invention.

Below, each category of compounds in the combination will be described separately. It is however to be understood that at least one compound from each category is present in the combination. The compounds can either be administered simultaneously or separately. The compounds in each category may be administered parenterally (such as intravenous, subcutaneous, or intra-muscular) or enterally (such as orally or through the gastrointestinal tract).

### RTEL1 Inhibitors

In one aspect, the first category of compound in the combination of the invention is an inhibitor targeting RTEL1. Such an inhibitor can be selected from the group consisting of, for example, small molecules, single stranded antisense oligonucleotide; siRNA molecule; or shRNA molecule

In the present section, the term “oligonucleotide” is to be understood as “oligonucleotide targeting RTEL1”.

Therapeutic oligonucleotides are potentially excellent RTEL1 inhibitors since they can target the RTEL1 transcript and promote its degradation either via the RNA interference pathway or via RNaseH cleavage. Alternatively, oligonucleotides such as aptamers can also act as inhibitors of RTEL1 protein interactions.

In one aspect, the first category of compound in the combination of the invention is an inhibitor targeting RTEL1. Such an inhibitor can be selected from the group of oligonucleotides consisting of single stranded antisense oligonucleotide; siRNA molecule; or shRNA molecule.

The present section describes oligonucleotides, or conjugates thereof, of the combination of the present invention; and suitable for use in treatment and/or prevention of Hepatitis B virus (HBV) infection; such as a chronic HBV infection, or in the treatment of cancer.

The oligonucleotides of the combination of the present invention are capable of inhibiting expression of RTEL1 in vitro and in vivo. The inhibition is achieved by hybridizing an oligonucleotide to a target nucleic acid encoding RTEL1 or which is involved in the regulation of RTEL1. The target nucleic acid may be a mammalian RTEL1 sequence, such as the sequence of SEQ ID NO: 1 and/or 2

In some embodiments of the invention, the oligonucleotide is capable of reducing cccDNA in an infected cell.

In some embodiments the oligonucleotide of the combination of the invention is capable of modulating the expression of the target by inhibiting or down-regulating it. Preferably, such modulation produces an inhibition of expression of at least 20% compared to the normal expression level of the target, more preferably at least 30%, at least 40%, at least 50%, at least 60%, at least 70%, at least 80%, or at least 90% inhibition compared to the normal expression level of the target. In some embodiments, the oligonucleotide may be capable of inhibiting expression levels of RTEL1 mRNA by at least 60% or 70% in vitro using 10 μM in PXB-PHH cells. In some embodiments of the invention, the oligonucleotide may be capable of inhibiting expression levels of RTEL1 protein by at least 50% in vitro using 10 μM PXB-PHH cells, this range of target reduction is advantageous in terms of selecting nucleic acid molecules with good correlation to the cccDNA reduction. Suitably, the examples provide assays that may be used to measure RTEL1 RNA or protein inhibition (e.g. example 1). The target inhibition is triggered by the hybridization between a contiguous nucleotide sequence of the oligonucleotide and the target nucleic acid. In some embodiments, the oligonucleotide comprises mismatches between the oligonucleotide and the target nucleic acid. Despite mismatches hybridization to the target nucleic acid may still be sufficient to show a desired inhibition of RTEL1 expression. Reduced binding affinity resulting from mismatches may advantageously be compensated by increased number of nucleotides in the oligonucleotide and/or an increased number of modified nucleosides capable of increasing the binding affinity to the target, such as 2′ sugar modified nucleosides, including LNA, present within the oligonucleotide sequence.

An aspect of the present invention relates to oligonucleotides of 12 to 60 nucleotides in length, which comprises a contiguous nucleotide sequence of at least 10 nucleotides in length, such as at least 12 to 30 nucleotides in length, which is at least 95% complementary, such as fully complementary, to a mammalian RTEL1 target nucleic acid, in particular a human RTEL1 nucleic acid. These oligonucleotides are capable of inhibiting the expression of RTEL1.

An aspect of the invention relates to an oligonucleotide which is an antisense oligonucleotide of 12 to 30 nucleotides in length, comprising a contiguous nucleotide sequence of at least 10 nucleotides, such as 10 to 30 nucleotides in length which is at least 90% complementary, such as fully complementary, to a mammalian RTEL1.

A further aspect of the present invention relates to an oligonucleotide comprising a contiguous nucleotide sequence of 12 to 20, such as 15 to 22, nucleotides in length with at least 90% complementarity, such as fully complementary, to the target nucleic acid of SEQ ID NO: 1.

In some embodiments, the oligonucleotide comprises a contiguous sequence of 10 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

It is advantageous if the oligonucleotide for use in the invention, or contiguous nucleotide sequence thereof, is fully complementary (100% complementary) to a region of the target nucleic acid, or in some embodiments may comprise one or two mismatches between the oligonucleotide and the target nucleic acid.

In some embodiments, the antisense oligonucleotide sequence is 100% complementary to a corresponding target nucleic acid of SEQ ID NO: 1.

In some embodiments of the invention, the oligonucleotide or the contiguous nucleotide sequence of the combination of the invention is at least 95% complementarity, such as fully (or 100%) complementary, to the target nucleic acid of SEQ ID NO: 1 and SEQ ID NO: 2.

In some embodiments, the oligonucleotide comprises a contiguous nucleotide sequence of 15 to 22 nucleotides in length with at least 90% complementary, such as 100% complementarity, to a corresponding target sequence present in SEQ ID NO: 1, wherein the target sequence is selected from the group consisting of SEQ ID NO: 3 to 26 (table 7) or region 1A to 959A in Table 8A.

Table 8A: Regions of SEQ ID NO 1 which may be targeted using an oligonucleotide of the combination of the invention

In some embodiments, the oligonucleotide comprises a contiguous nucleotide sequence of 16 to 20, such as 15 to 22, nucleotides in length with at least 90% complementary, such as 100% complementarity, to a corresponding target sequence present in SEQ ID NO: 1, wherein the target sequence is selected from the group consisting of SEQ ID NO: 3 to 26 (table 7) or region B1 to B28 in Table 8B.

In some embodiments of the invention, the oligonucleotide comprises or consists of 12 to 60 nucleotides in length, such as from 13 to 50, such as from 14 to 35, such as 15 to 30, such as from 16 to 20 contiguous nucleotides in length. In a preferred embodiment, the oligonucleotide comprises or consists of 15, 16, 17, 18, 19 or 20 nucleotides in length.

In some embodiments, the contiguous nucleotide sequence of the oligonucleotide which is complementary to the target nucleic acids comprises or consists of 12 to 30, such as from 13 to 25, such as from 15 to 23, such as from 16 to 22, contiguous nucleotides in length.

In some embodiments, the contiguous nucleotide sequence of the siRNA or shRNA which is complementary to the target nucleic acids comprises or consists of 18 to 28, such as from 19 to 26, such as from 20 to 24, such as from 21 to 23, contiguous nucleotides in length.

In some embodiments, the contiguous nucleotide sequence of the single stranded antisense oligonucleotide which is complementary to the target nucleic acids comprises or consists of 12 to 22, such as from 14 to 20, such as from 16 to 20, such as from 15 to 21, such as from 15 to 18, such as from 16 to 18, such as from 16 to 17 contiguous nucleotides in length.

In some embodiments, the oligonucleotide or contiguous nucleotide sequence comprises or consists of a sequence selected from the group consisting of sequences listed in table 9A

In some embodiments, the oligonucleotide or contiguous nucleotide sequence comprises or consists of 10 to 30 nucleotides in length with at least 90% identity, preferably 100% identity, to a sequence selected from the group consisting of SEQ ID NO: 27 to 246 (see motif sequences listed in table 9A). In a particular embodiment the oligonucleotide or contiguous nucleotide sequence is selected from SEQ ID NO: 27; 28; 29; 30; 31; 32; 33; 34; 37; 40; 41; 42; 43; 44; 45; 46; 47; 48; 51; 54; 88; 114; 135; 208; 237; 243; 244; 245 and 246.

In a particular embodiment the oligonucleotide or contiguous nucleotide sequence is SEQ ID NO: 243

In a particular embodiment the oligonucleotide or contiguous nucleotide sequence is SEQ ID NO: 244

In a particular embodiment the oligonucleotide or contiguous nucleotide sequence is SEQ ID NO: 245

In a particular embodiment the oligonucleotide or contiguous nucleotide sequence is SEQ ID NO: 246

It is understood that the contiguous oligonucleotide sequence (motif sequence) can be modified to, for example, increase nuclease resistance and/or binding affinity to the target nucleic acid.

The pattern in which the modified nucleosides (such as high affinity modified nucleosides) are incorporated into the oligonucleotide sequence is generally termed oligonucleotide design.

The oligonucleotide may be designed with modified nucleosides and RNA nucleosides (in particular for siRNA and shRNA molecules) or DNA nucleosides (in particular for single stranded antisense oligonucleotides). Advantageously, high affinity modified nucleosides are used.

In an embodiment, the oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide comprises one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In a further embodiment the oligonucleotide comprises at least one modified internucleoside linkage. Suitable internucleoside modifications are described in the “Definitions” section under “Modified internucleoside linkage”. It is advantageous if at least 2 to 3 internucleoside linkages at the 5′ or 3′ end of the oligonucleotide are phosphorothioate internucleoside linkages. For single stranded antisense oligonucleotides it is advantageous if at least 75%, such as all, the internucleoside linkages within the contiguous nucleotide sequence are phosphorothioate internucleoside linkages. In some embodiments all the internucleotide linkages in the contiguous sequence of the single stranded antisense oligonucleotide are phosphorothioate linkages.

In some embodiments of the invention, the oligonucleotide comprises at least one LNA nucleoside, such as 1, 2, 3, 4, 5, 6, 7, or 8 LNA nucleosides, such as from 2 to 6 LNA nucleosides, such as from 3 to 7 LNA nucleosides, 4 to 8 LNA nucleosides or 3, 4, 5, 6, 7 or 8 LNA nucleosides. In some embodiments, at least 75% of the modified nucleosides in the oligonucleotide are LNA nucleosides, such as 80%, such as 85%, such as 90% of the modified nucleosides are LNA nucleosides. In a still further embodiment all the modified nucleosides in the oligonucleotide are LNA nucleosides. In a further embodiment, the oligonucleotide may comprise both beta-D-oxy-LNA, and one or more of the following LNA nucleosides: thio-LNA, amino-LNA, oxy-LNA, ScET and/or ENA in either the beta-D or alpha-L configurations or combinations thereof. In a further embodiment, all LNA cytosine units are 5-methyl-cytosine. It is advantageous for the nuclease stability of the oligonucleotide or contiguous nucleotide sequence to have at least 1 LNA nucleoside at the 5′ end and at least 2 LNA nucleosides at the 3′ end of the nucleotide sequence.

In an embodiment of the invention, the oligonucleotide is capable of recruiting RNase H.

In the current invention, an advantageous structural design is a gapmer design as described in the “Definitions” section under for example “Gapmer”, “LNA Gapmer” and “MOE gapmer”. In the present invention, it is advantageous if the antisense oligonucleotide is a gapmer with an F-G-F′ design. In some embodiments, the gapmer is an LNA gapmer with uniform flanks.

In classic gapmer design, i.e.gapmers with uniform flanks (e.g. 4-12-2), all the nucleotides in the flanks (F and F′) are constituted of the same type of 2′-sugar modified nucleoside, e.g. LNA, CET, or MOE, and a stretch of DNA in the middle forming the gap (G). In gapmers with alternating flank designs, the flanks of oligonucleotide are annotated as a series of integers, representing a number of beta-D-oxy LNA nucleosides (L) followed by a number of DNA nucleosides (D). For example, a flank F′ with a 1-2-1-1-3 motif represents LDDLDLLL (see CMP ID NO 246_1; Table 9A or 9B). Both flanks have a beta-D-oxy LNA nucleoside at the 5′ and 3′ terminal. The gap region (G), which is constituted of a number of DNA nucleosides is located between the flanks.

In some embodiments of the invention, the LNA gapmer is selected from the following flank designs: 2-12-3, 4-14-2, 3-10-3, 3-9-3, 2-15-2, 2-12-4, 1-13-2, 3-13-2, 4-13-2, 2-12-2, 3-12-2, 3-15-2, 3-14-2, 3-13-3, 2-14-4, 3-12-3, 1-14-3, 3-14-3, 2-14-3, 2-15-3, 3-11-3, 1-12-3, 1-11-4, 1-13-2, 2-13-2, 2-16-2, 1-14-2, 1-17-3, 1-18-2, 4-12-2, 2-13-4, 2-11-1-2-1-1-3, and 2-17- 4.

TABLE 9A
list of oligonucleotide motif sequences (indicated by SEQ ID NO),
designs of these, as well as specific oligonucleotide compounds
(indicated by CMP ID NO) designed based onthe motif sequence.

| | | | | | Start |
|---|---|---|---|---|---|
| | | | | | position |
| SEQ | | | | CMP | on SEQ |
| ID NO | Motif sequence | Design | Oligonucleotide Compound | ID NO | ID NO: 1 |
| 27 | catggaaggacagtggt | 2-12-3 | CAtggaaggacagtGGT | 27_1 | 8295 |
| 28 | agctttattataacttgaat | 4-14-2 | AGCTttattataacttgaAT | 28_1 | 8684 |
| 29 | cgggacaggtagtaag | 3-10-3 | CGGgacaggtagtAAG | 29_1 | 9668 |
| 30 | cgggacaggtagtaa | 3-9-3 | CGGgacaggtagTAA | 30_1 | 9669 |
| 31 | gcatccaacaagtaattgt | 2-15-2 | GCatccaacaagtaattGT | 31_1 | 9722 |
| 32 | gcatccaacaagtaattg | 2-12-4 | GCatccaacaagtaATTG | 32_1 | 9723 |
| 33 | ggcatccaacaagtaatt | 3-13-2 | GGCatccaacaagtaaTT | 33_1 | 9724 |
| 34 | ggttgggttagaagct | 2-12-2 | GGttgggttagaagCT | 34_1 | 10921 |
| 35 | gcttttacatttaggtttat | 3-15-2 | GCTtttacatttaggtttAT | 35_1 | 11483 |
| 36 | catgttcctttctataact | 3-14-2 | CATgttcctttctataaCT | 36_1 | 11512 |
| 37 | agctttaaattttggtgaa | 3-13-3 | AGCtttaaattttggtGAA | 37_1 | 11622 |
| 38 | ttttacatactctggtcaaa | 2-14-4 | TTttacatactctggtCAAA | 38_1 | 11753 |
| 39 | ttttacatactctggtca | 3-12-3 | TTTtacatactctggTCA | 39_1 | 11755 |
| 40 | gaattttacatactctggtc | 3-14-3 | GAAttttacatactctgGTC | 40_1 | 11756 |
| 41 | gaattttacatactctggt | 2-14-3 | GAattttacatactctGGT | 41_1 | 11757 |
| 42 | gagaattttacatactctgg | 2-15-3 | GAgaattttacatactcTGG | 42_1 | 11758 |
| 43 | atctttgaacacgtctt | 3-11-3 | ATCtttgaacacgtCTT | 43_1 | 12868 |
| 44 | acaaaaaacagtaggtcc | 2-12-4 | ACaaaaaacagtagGTCC | 44_1 | 13234 |
| 45 | ggaataaaacagtaggtc | 2-12-4 | GGaataaaacagtaGGTC | 45_1 | 13551 |
| 46 | agcttcgtcaaagatcac | 3-13-2 | AGCttcgtcaaagatcAC | 46_1 | 14786 |
| 47 | ggtgggtggatgtttc | 1-12-3 | GgtgggtggatgtTTC | 47_1 | 18085 |
| 47 | ggtgggggatgtttc | 1-11-4 | GgtgggtggatgTTTC | 47_2 | 18085 |
| 47 | ggtgggtggatgtttc | 1-13-2 | GgtgggtggatgttTC | 47_3 | 18085 |
| 48 | ggtggtgtggagaagc | 1-12-3 | GgtggtgtggagaAGC | 48_1 | 22425 |
| 48 | ggtggtgtggagaagc | 1-13-2 | GgtggtgtggagaaGC | 48_2 | 22425 |
| 49 | gctcatactccacacac | 2-13-2 | GCtcatactccacacAC | 49_1 | 33030 |
| 50 | catcggaacccttgtagtcc | 2-16-2 | CAtcggaacccttgtagtCC | 50_1 | 35103 |
| 51 | gatacagacctcctcaaac | 2-15-2 | GAtacagacctcctcaaAC | 51_1 | 35371 |
| 52 | ggtggaggtggtgctgc | 1-14-2 | GgtggaggtggtgctGC | 52_1 | 35636 |
| 53 | aggtggaggtggtgct | 1-12-3 | AggtggaggtggtGCT | 53_1 | 35638 |
| 54 | tggtgtgggagtagca | 2-12-2 | TGgtgtgggagtagCA | 54_1 | 36915 |
| 55 | cgatggcgagaaatta | 4-10-2 | CGATggcgagaaatTA | 55_1 | 3824 |
| 56 | taattcagcaaaaaagccca | 3-15-2 | TAAttcagcaaaaaagccCA | 56_1 | 3858 |
| 57 | aagaatctgacacccca | 2-12-3 | AAgaatctgacaccCCA | 57_1 | 3924 |
| 58 | agacagccaagaatctgacac | 1-18-2 | AgacagccaagaatctgacAC | 58_1 | 3928 |
| 59 | cagccaagaatctgaca | 2-12-3 | CAgccaagaatctgACA | 59_1 | 3929 |
| 60 | acaggaacccgacag | 2-10-3 | ACaggaacccgaCAG | 60_1 | 4496 |
| 61 | gttactctcttgtttcttcac | 1-18-2 | GttactctcttgtttcttcAC | 61_1 | 4789 |
| 62 | ttactctcttgtttcttca | 1-16-2 | TtactctcttgtttcttCA | 62_1 | 4790 |
| 62 | ttactctcttgtttcttca | 1-14-4 | TtactctcttgtttcTTCA | 62_2 | 4790 |
| 63 | gttactctcttgtttcttc | 2-15-2 | GTtactctcttgtttctTC | 63_1 | 4791 |
| 64 | cgtgggtggagaagca | 1-13-2 | CgtgggtggagaagCA | 64_1 | 5717 |
| 65 | acgtgggtggagaagc | 2-12-2 | ACgtgggtggagaaGC | 65_1 | 5718 |
| 66 | cagaaactgtaagggca | 1-13-3 | CagaaactgtaaggGCA | 66_1 | 5815 |
| 67 | agggatagcagggaagg | 2-13-2 | AGggatagcagggaaGG | 67_1 | 7246 |
| 68 | gcttaaacacagacaga | 2-11-4 | GCttaaacacagaCAGA | 68_1 | 7501 |
| 69 | tgcttaaacacagacag | 3-11-3 | TGCttaaacacagaCAG | 69_1 | 7502 |
| 70 | cagggcagggaagaacag | 1-14-3 | CagggcagggaagaaCAG | 70_1 | 7845 |
| 71 | catggaaggacagtgg | 3-10-3 | CATggaaggacagTGG | 71_1 | 8296 |
| 71 | catggaaggacagtgg | 1-12-3 | CatggaaggacagTGG | 71_2 | 8296 |
| 72 | ccccctcaatataagaa | 3-12-2 | CCCcctcaatataagAA | 72_1 | 8705 |
| 73 | aaccaaccctattcctgg | 2-14-2 | AAccaaccctattcctGG | 73_1 | 9375 |
| 73 | aaccaaccctattcctgg | 1-15-2 | AaccaaccctattcctGG | 73_2 | 9375 |
| 74 | accaaccctattcctg | 1-12-3 | AccaaccctattcCTG | 74_1 | 9376 |
| 75 | aaccaaccctattoctg | 3-12-2 | AACcaaccctattccTG | 75_1 | 9376 |
| 76 | aaaaccaaccctattcct | 3-12-3 | AAAaccaaccctattCCT | 76_1 | 9377 |
| 77 | aaaccaaccctattcc | 4-10-2 | AAACcaaccctattCC | 77_1 | 9378 |
| 78 | ggtagtaagggcacacc | 1-14-2 | GgtagtaagggcacaCC | 78_1 | 9660 |
| 79 | gacaggtagtaagggcacac | 1-17-2 | GacaggtagtaagggcacAC | 79_1 | 9661 |
| 80 | gtagtaagggcacac | 3-9-3 | GTAgtaagggcaCAC | 80_1 | 9661 |
| 81 | gacaggtagtaagggcaca | 1-16-2 | GacaggtagtaagggcaCA | 81_1 | 9662 |
| 82 | acaggtagtaagggcaca | 2-14-2 | ACaggtagtaagggcaCA | 82_1 | 9662 |
| 83 | gacaggtagtaagggca | 2-13-2 | GAcaggtagtaagggCA | 83_1 | 9664 |
| 84 | cgggacaggtagtaaggg | 1-15-2 | CgggacaggtagtaagGG | 84_1 | 9666 |
| 85 | catccaacaagtaattgt | 3-12-3 | CATccaacaagtaatTGT | 85_1 | 9722 |
| 85 | catccaacaagtaattgt | 2-13-3 | CAtccaacaagtaatTGT | 85_2 | 9722 |
| 86 | ggcatccaacaagtaattgt | 1-16-3 | GgcatccaacaagtaatTGT | 86_1 | 9722 |
| 87 | ggcatccaacaagtaattg | 3-14-2 | GGCatccaacaagtaatTG | 87_1 | 9723 |
| 87 | ggcatccaacaagtaattg | 1-15-3 | GgcatccaacaagtaaTTG | 87_2 | 9723 |
| 88 | ggcatccaacaagtaat | 4-11-2 | GGCAtccaacaagtaAT | 88_1 | 9725 |
| 88 | ggcatccaacaagtaat | 3-12-2 | GGCatccaacaagtaAT | 88_2 | 9725 |
| 89 | cgtgaaggagagaacct | 2-12-3 | CGtgaaggagagaaCCT | 89_1 | 10036 |
| 90 | acgtgaaggagagaacc | 3-12-2 | ACGtgaaggagagaaCC | 90_1 | 10037 |
| 90 | gacgtgaaggagagaacc | 2-13-3 | GAcgtgaaggagagaACC | 90_2 | 10037 |
| 91 | gacgtgaaggagagaacc | 2-14-2 | GAcgtgaaggagagaaCC | 91_1 | 10037 |
| 91 | acgtgaaggagagaacc | 4-11-2 | ACGTgaaggagagaaCC | 91_2 | 10037 |
| 92 | gacgtgaaggagagaac | 2-11-4 | GAcgtgaaggagaGAAC | 92_1 | 10038 |
| 93 | cagtcttgctatgcct | 2-12-2 | CAgtcttgctatgcCT | 93_1 | 10563 |
| 94 | ctagaatcaaagctcca | 2-12-3 | CTagaatcaaagctCCA | 94_1 | 10591 |
| 95 | acatcgcacttgggc | 1-12-2 | AcatcgcacttggGC | 95_1 | 10705 |
| 96 | cacggcaaacctcacc | 1-12-3 | CacggcaaacctcACC | 96_1 | 10851 |
| 97 | aaccacggcaaacctcac | 3-13-2 | AACcacggcaaacctcAC | 97_1 | 10852 |
| 98 | caaagcaccgagtcacc | 1-13-3 | CaaagcaccgagtcACC | 98_1 | 10873 |
| 99 | tcaaagcaccgagtcac | 1-13-3 | TcaaagcaccgagtCAC | 99_1 | 10874 |
| 100 | ctggttgggttagaag | 2-10-4 | CTggttgggttaGAAG | 100_1 | 10923 |
| 100 | ctggttgggttagaag | 2-12-2 | CTggttgggttagaAG | 100_2 | 10923 |
| 101 | tataacttttagtttagc | 2-12-4 | TAtaacttttagttTAGC | 101_1 | 11501 |
| 102 | ttcctttctataactttt | 4-12-2 | TTCCtttctataacttTT | 102_1 | 11509 |
| 103 | gttcctttctataactttt | 4-13-2 | GTTCctttctataacttTT | 103_1 | 11509 |
| 104 | gttcctttctataacttt | 4-12-2 | GTTCctttctataactTT | 104_1 | 11510 |
| 105 | atgttcctttctataacttt | 2-15-3 | ATgttcctttctataacTTT | 105_1 | 11510 |
| 106 | atgttcctttctataactt | 2-14-3 | ATgttcctttctataaCTT | 106_1 | 11511 |
| 107 | atgttcctttctataact | 2-14-2 | ATgttcctttctataaCT | 107_1 | 11512 |
| 108 | gctttaatctgccttc | 1-11-4 | GctttaatctgcCTTC | 108_1 | 12697 |
| 109 | ccgtggctttaatctgc | 1-14-2 | CcgtggctttaatctGC | 109_1 | 12701 |
| 110 | ccgtggctttaatctg | 2-12-2 | CCgtggctttaatcTG | 110_1 | 12702 |
| 110 | ccgtggctttaatctg | 3-11-2 | CCGtggctttaatcTG | 110_2 | 12702 |
| 111 | caaaaaacagtaggtcc | 2-11-4 | CAaaaaacagtagGTCC | 111_1 | 13234 |
| 111 | caaaaaacagtaggtcc | 3-11-3 | CAAaaaacagtaggTCC | 111_2 | 13234 |
| 112 | gaataaaacagtaggtcc | 2-12-4 | GAataaaacagtagGTCC | 112_1 | 13550 |
| 113 | ggaataaaacagtaggtcc | 4-13-2 | GGAAtaaaacagtaggtCC | 113_1 | 13550 |
| 113 | ggaataaaacagtaggtcc | 2-15-2 | GGaataaaacagtaggtCC | 113_2 | 13550 |
| 113 | ggaataaaacagtaggtcc | 1-14-4 | GgaataaaacagtagGTCC | 113_3 | 13550 |
| 114 | ggaataaaacagtaggt | 3-11-3 | GGAataaaacagtaGGT | 114_1 | 13552 |
| 114 | ggaataaaacagtaggt | 2-11-4 | GGaataaaacagtAGGT | 114_2 | 13552 |
| 115 | ggaataaaacagtagg | 2-10-4 | GGaataaaacagTAGG | 115_1 | 13553 |
| 116 | cacagagtgtcatggg | 1-13-2 | CacagagtgtcatgGG | 116_1 | 14032 |
| 117 | acagcatggaaaggcacg | 1-13-4 | AcagcatggaaaggCACG | 117_1 | 14523 |
| 118 | cagcatggaaaggcacg | 1-12-4 | CagcatggaaaggCACG | 118_1 | 14523 |
| 119 | tacaggaggaagagaagggac | 1-18-2 | TacaggaggaagagaagggAC | 119_1 | 14725 |
| 120 | acaggaggaagagaaggg | 1-13-4 | AcaggaggaagagaAGGG | 120_1 | 14727 |
| 121 | tctacaggaggaagagaa | 4-12-2 | TCTAcaggaggaagagAA | 121_1 | 14730 |
| 121 | tctacaggaggaagagaa | 1-13-4 | TctacaggaggaagAGAA | 121_2 | 14730 |
| 122 | tctacaggaggaagaga | 4-11-2 | TCTAcaggaggaagaGA | 122_1 | 14731 |
| 122 | tctacaggaggaagaga | 2-12-3 | TCtacaggaggaagAGA | 122_2 | 14731 |
| 122 | tctacaggaggaagaga | 2-11-4 | TCtacaggaggaaGAGA | 122_3 | 14731 |
| 123 | cttcgtcaaagatcacg | 2-11-4 | CTtcgtcaaagatCACG | 123_1 | 14785 |
| 124 | gcttcgtcaaagatcacg | 2-13-3 | GCttcgtcaaagatcACG | 124_1 | 14785 |
| 125 | gcttcgtcaaagatcac | 3-11-3 | GCTtcgtcaaagatCAC | 125_1 | 14786 |
| 125 | gcttcgtcaaagatcac | 2-13-2 | GCttcgtcaaagatcAC | 125_2 | 14786 |
| 126 | ccagaaaggtttgcg | 3-10-2 | CCAgaaaggtttgCG | 126_1 | 14874 |
| 127 | tccagaaaggtttgcg | 3-11-2 | TCCagaaaggtttgCG | 127_1 | 14874 |
| 127 | tccagaaaggtttgcg | 1-12-3 | TccagaaaggtttGCG | 127_2 | 14874 |
| 128 | cagaggcatcggatcag | 2-13-2 | CAgaggcatcggatcAG | 128_1 | 14974 |
| 129 | cagaggcatcggatca | 3-11-2 | CAGaggcatcggatCA | 129_1 | 14975 |
| 130 | agcagaggcatcggatc | 2-13-2 | AGcagaggcatcggaTC | 130_1 | 14976 |
| 131 | attcttcacacatcttc | 2-11-4 | ATtcttcacacatCTTC | 131_1 | 16133 |
| 132 | ctatgaacgcacctg | 3-9-3 | CTAtgaacgcacCTG | 132_1 | 16282 |
| 133 | ggctatgaacgcacctg | 1-14-2 | GgctatgaacgcaccTG | 133_1 | 16282 |
| 134 | gctgggagaagacatag | 1-12-4 | GctgggagaagacATAG | 134_1 | 16593 |
| 135 | caaaatgcccttacagtga | 4-13-2 | CAAAatgcccttacagtGA | 135_1 | 16919 |
| 136 | caaaatgcccttacagt | 2-12-3 | CAaaatgcccttacAGT | 136_1 | 16921 |
| 137 | tgtgcgattttaaaggaaaat | 3-15-3 | TGTgcgattttaaaggaaAAT | 137_1 | 17525 |
| 138 | catgtgcgattttaaaggaaa | 3-15-3 | CATgtgcgattttaaaggAAA | 138_1 | 17527 |
| 139 | tgtgcgattttaaaggaa | 4-12-2 | TGTGcgattttaaaggAA | 139_1 | 17528 |
| 140 | catgtgcgattttaaagga | 1-15-3 | CatgtgcgattttaaaGGA | 140_1 | 17529 |
| 141 | atgtgcgattttaaagga | 3-13-2 | ATGtgcgattttaaagGA | 141_1 | 17529 |
| 142 | accctgtcacttaaatatatg | 1-18-2 | AccctgtcacttaaatataTG | 142_1 | 17712 |
| 143 | gagggaggtggagcgtt | 1-14-2 | GagggaggtggagcgTT | 143_1 | 17924 |
| 144 | ctgaagagtggagaagg | 2-11-4 | CTgaagagtggagAAGG | 144_1 | 18130 |
| 144 | ctgaagagtggagaagg | 1-13-3 | CtgaagagtggagaAGG | 144_2 | 18130 |
| 145 | caataaataaagtgtgagga | 3-14-3 | CAAtaaataaagtgtgaGGA | 145_1 | 18454 |
| 146 | caacccagtaaccatgac | 3-13-2 | CAAcccagtaaccatgAC | 146_1 | 19424 |
| 147 | caacccagtaaccatga | 3-12-2 | CAAcccagtaaccatGA | 147_1 | 19425 |
| 148 | accaacccagtaaccatga | 1-16-2 | AccaacccagtaaccatGA | 148_1 | 19425 |
| 149 | gagcaggtgttttatc | 3-11-2 | GAGcaggtgttttaTC | 149_1 | 19825 |
| 150 | ggtcgaggaggtgtcac | 1-14-2 | GgtcgaggaggtgtcAC | 150_1 | 20437 |
| 150 | ggtcgaggaggtgtcac | 2-13-2 | GGtcgaggaggtgtcAC | 150_2 | 20437 |
| 151 | gtcgaggaggtgtcac | 1-11-4 | GtcgaggaggtgTCAC | 151_1 | 20437 |
| 152 | ggtcgaggaggtgtca | 1-13-2 | GgtcgaggaggtgtCA | 152_1 | 20438 |
| 152 | ggtcgaggaggtgtca | 1-12-3 | GgtcgaggaggtgTCA | 152_2 | 20438 |
| 153 | ggtcgaggaggtgtc | 2-11-2 | GGtcgaggaggtgTC | 153_1 | 20439 |
| 154 | ccaggtctcaaaaaggg | 1-13-3 | CcaggtctcaaaaaGGG | 154_1 | 20653 |
| 155 | attacgctgaggaca | 1-10-4 | AttacgctgagGACA | 155_1 | 21489 |
| 156 | cattacgctgaggac | 4-9-2 | CATTacgctgaggAC | 156_1 | 21490 |
| 157 | cttgagcattacgc | 3-8-3 | CTTgagcattaCGC | 157_1 | 21497 |
| 158 | cgaggagaagaaggcag | 3-12-2 | CGAggagaagaaggcAG | 158_1 | 22019 |
| 158 | cgaggagaagaaggcag | 2-11-4 | CGaggagaagaagGCAG | 158_2 | 22019 |
| 159 | ccttggtctgaaacgtgat | 1-15-3 | CcttggtctgaaacgtGAT | 159_1 | 22071 |
| 160 | ctaacgcctccacgc | 1-12-2 | CtaacgcctccacGC | 160_1 | 22281 |
| 161 | ggacaggctctacgg | 1-11-3 | GgacaggctctaCGG | 161_1 | 22312 |
| 162 | actaatacagcaggagaagg | 2-16-2 | ACtaatacagcaggagaaGG | 162_1 | 22964 |
| 163 | aactaatacagcaggagaagg | 1-16-4 | AactaatacagcaggagAAGG | 163_1 | 22964 |
| 163 | aactaatacagcaggagaagg | 1-17-3 | AactaatacagcaggagaAGG | 163_2 | 22964 |
| 164 | taactaatacagcaggagaag | 1-16-4 | TaactaatacagcaggaGAAG | 164_1 | 22965 |
| 165 | ttgaagagccaaccac | 1-11-4 | TtgaagagccaaCCAC | 165_1 | 24131 |
| 166 | ccattttcactgtcaag | 3-12-2 | CCAttttcactgtcaAG | 166_1 | 25605 |
| 167 | gccattttcactgtcaa | 2-12-3 | GCcattttcactgtCAA | 167_1 | 25606 |
| 168 | agaaatgcggagaagc | 2-10-4 | AGaaatgcggagAAGC | 168_1 | 25796 |
| 169 | aaatggaaaaaatgaccagc | 2-14-4 | AAatggaaaaaatgacCAGC | 169_1 | 26188 |
| 170 | aggacttacgacaaaaccac | 1-15-4 | AggacttacgacaaaaCCAC | 170_1 | 26505 |
| 171 | ggacttacgacaaaacca | 2-13-3 | GGacttacgacaaaaCCA | 171_1 | 26506 |
| 172 | gacttacgacaaaacca | 3-11-3 | GACttacgacaaaaCCA | 172_1 | 26506 |
| 173 | acaccaggacttacgaca | 1-14-3 | AcaccaggacttacgACA | 173_1 | 26512 |
| 174 | tagaaattcaacatggc | 1-12-4 | TagaaattcaacaTGGC | 174_1 | 27376 |
| 174 | tagaaattcaacatggc | 4-11-2 | TAGAaattcaacatgGC | 174_2 | 27376 |
| 174 | tagaaattcaacatggc | 2-12-3 | TAgaaattcaacatGGC | 174_3 | 27376 |
| 175 | ctagaaattcaacatggc | 2-13-3 | CTagaaattcaacatGGC | 175_1 | 27376 |
| 176 | gtcatcggttcacc | 1-9-4 | GtcatcggttCACC | 176_1 | 27602 |
| 177 | actcgaagacgcca | 2-8-4 | ACtcgaagacGCCA | 177_1 | 28539 |
| 178 | gactcgaagacgcc | 3-9-2 | GACtcgaagacgCC | 178_1 | 28540 |
| 179 | ggcacaagcagaacgac | 2-13-2 | GGcacaagcagaacgAC | 179_1 | 29235 |
| 180 | agtcagaacaaaggaggc | 1-15-2 | AgtcagaacaaaggagGC | 180_1 | 29668 |
| 181 | gaagtcagaacaaaggag | 4-12-2 | GAAGtcagaacaaaggAG | 181_1 | 29670 |
| 182 | gcagaagtcagaacaaagg | 1-14-4 | GcagaagtcagaacaAAGG | 182_1 | 29672 |
| 183 | gtgcagaagtcagaacaaa | 3-13-3 | GTGcagaagtcagaacAAA | 183_1 | 29674 |
| 183 | gtgcagaagtcagaacaaa | 3-14-2 | GTGcagaagtcagaacaAA | 183_2 | 29674 |
| 184 | gtgcagaagtcagaacaa | 3-13-2 | GTGcagaagtcagaacAA | 184_1 | 29675 |
| 185 | aaggatgagggagcggac | 1-14-3 | AaggatgagggagcgGAC | 185_1 | 29894 |
| 186 | gtaaggatgagggagc | 2-12-2 | GTaaggatgagggaGC | 186_1 | 29898 |
| 187 | tggtaaggatgagggag | 1-12-4 | TggtaaggatgagGGAG | 187_1 | 29899 |
| 188 | cgtacatctgcatctc | 2-10-4 | CGtacatctgcaTCTC | 188_1 | 29951 |
| 189 | tgtaagataagaggcaacact | 1-18-2 | TgtaagataagaggcaacaCT | 189_1 | 30947 |
| 190 | ttgtaagataagaggcaacac | 1-17-3 | TtgtaagataagaggcaaCAC | 190_1 | 30948 |
| 191 | ttgtaagataagaggcaaca | 2-14-4 | TTgtaagataagaggcAACA | 191_1 | 30949 |
| 192 | tttgtaagataagaggcaaca | 2-17-2 | TTtgtaagataagaggcaaCA | 192_1 | 30949 |
| 193 | tgtaagataagaggcaa | 2-11-4 | TGtaagataagagGCAA | 193_1 | 30951 |
| 194 | ctggaaggaaagttggt | 2-12-3 | CTggaaggaaagttGGT | 194_1 | 31229 |
| 195 | atagtaagcactgatggtc | 3-14-2 | ATAgtaagcactgatggTC | 195_1 | 31245 |
| 195 | atagtaagcactgatggtc | 1-14-4 | AtagtaagcactgatGGTC | 195_2 | 31245 |
| 196 | tagtaagcactgatgg | 2-11-3 | TAgtaagcactgaTGG | 196_1 | 31247 |
| 197 | catagtaagcactgatg | 3-12-2 | CATagtaagcactgaTG | 197_1 | 31248 |
| 197 | catagtaagcactgatg | 2-11-4 | CAtagtaagcactGATG | 197_2 | 31248 |
| 198 | ctgtaactcacctggc | 1-13-2 | CtgtaactcacctgGC | 198_1 | 31835 |
| 198 | ctgtaactcacctggc | 2-12-2 | CTgtaactcacctgGC | 198_2 | 31835 |
| 199 | cggatcactcgcccg | 1-12-2 | CggatcactcgccCG | 199_1 | 32000 |
| 200 | acacaggctactctcgg | 1-14-2 | AcacaggctactctcGG | 200_1 | 33017 |
| 201 | acacaggctactctcg | 3-10-3 | ACAcaggctactcTCG | 201_1 | 33018 |
| 202 | ccacacacaggctactc | 1-14-2 | CcacacacaggctacTC | 202_1 | 33021 |
| 203 | atactccacacacaggct | 1-15-2 | AtactccacacacaggCT | 203_1 | 33025 |
| 204 | atactccacacacaggc | 1-14-2 | AtactccacacacagGC | 204_1 | 33026 |
| 205 | gctcatactccacacacag | 1-16-2 | GctcatactccacacacAG | 205_1 | 33028 |
| 206 | tcatactccacacacag | 2-11-4 | TCatactccacacACAG | 206_1 | 33028 |
| 206 | tcatactccacacacag | 2-13-2 | TCatactccacacacAG | 206_2 | 33028 |
| 207 | gctcatactccacacaca | 1-14-3 | GctcatactccacacACA | 207_1 | 33029 |
| 207 | gctcatactccacacaca | 1-15-2 | GctcatactccacacaCA | 207_2 | 33029 |
| 208 | tgctcatactccacacac | 1-14-3 | TgctcatactccacaCAC | 208_1 | 33030 |
| 208 | tgctcatactccacacac | 1-15-2 | TgctcatactccacacAC | 208_2 | 33030 |
| 208 | tgctcatactccacacac | 2-14-2 | TGctcatactccacacAC | 208_3 | 33030 |
| 209 | agcaggaagcagggagaaa | 2-15-2 | AGcaggaagcagggagaAA | 209_1 | 33562 |
| 210 | tccgaccacagcgag | 2-11-2 | TCcgaccacagcgAG | 210_1 | 33681 |
| 211 | cagaagccaagggacatg | 1-14-3 | CagaagccaagggacATG | 211_1 | 34432 |
| 211 | cagaagccaagggacatg | 2-14-2 | CAgaagccaagggacaTG | 211_2 | 34432 |
| 212 | cagaagccaagggacat | 2-12-3 | CAgaagccaagggaCAT | 212_1 | 34433 |
| 213 | ccagaccaacacggaaacg | 1-14-4 | CcagaccaacacggaAACG | 213_1 | 34571 |
| 214 | ccagaccaacacggaaac | 2-12-4 | CCagaccaacacggAAAC | 214_1 | 34572 |
| 215 | gaatgggcaaagggtaga | 4-12-2 | GAATgggcaaagggtaGA | 215_1 | 34742 |
| 215 | aatgggcaaagggtaga | 2-12-3 | AAtgggcaaagggtAGA | 215_2 | 34742 |
| 216 | gaatgggcaaagggtaga | 2-14-2 | GAatgggcaaagggtaGA | 216_1 | 34742 |
| 217 | gaatgggcaaagggtag | 2-12-3 | GAatgggcaaagggTAG | 217_1 | 34743 |
| 218 | gaacccttgtagtcctg | 1-14-2 | GaacccttgtagtccTG | 218_1 | 35101 |
| 219 | aacccttgtagtcct | 4-9-2 | AACCcttgtagtcCT | 219_1 | 35102 |
| 220 | ggaacccttgtagtc | 2-11-2 | GGaacccttgtagTC | 220_1 | 35104 |
| 221 | atcggaacccttgtagtc | 2-14-2 | ATcggaacccttgtagTC | 221_1 | 35104 |
| 221 | atcggaacccttgtagtc | 1-15-2 | AtcggaacccttgtagTC | 221_2 | 35104 |
| 222 | catcggaacccttgtagtc | 1-16-2 | CatcggaacccttgtagTC | 222_1 | 35104 |
| 223 | catcggaacccttgtagt | 2-14-2 | CAtcggaacccttgtaGT | 223_1 | 35105 |
| 224 | gatacagacctcctcaaact | 1-17-2 | GatacagacctcctcaaaCT | 224_1 | 35370 |
| 225 | gatacagacctcctcaaac | 2-14-3 | GAtacagacctcctcaAAC | 225_1 | 35371 |
| 226 | gatacagacctcctcaaa | 2-13-3 | GAtacagacctcctcAAA | 226_1 | 35372 |
| 226 | gatacagacctcctcaaa | 2-12-4 | GAtacagacctcctCAAA | 226_2 | 35372 |
| 226 | gatacagacctcctcaaa | 1-13-4 | GatacagacctcctCAAA | 226_3 | 35372 |
| 227 | gatacagacctcctcaa | 2-11-4 | GAtacagacctccTCAA | 227_1 | 35373 |
| 227 | gatacagacctcctcaa | 1-13-3 | GatacagacctcctCAA | 227_2 | 35373 |
| 228 | gccccatttaccagtg | 1-13-2 | GccccatttaccagTG | 228_1 | 35470 |
| 229 | cccaacaagtgatgct | 2-12-2 | CCcaacaagtgatgCT | 229_1 | 35965 |
| 230 | cccaacaagtgatgc | 2-11-2 | CCcaacaagtgatGC | 230_1 | 35966 |
| 231 | gtaccaagcccagaagg | 1-14-2 | GtaccaagcccagaaGG | 231_1 | 36279 |
| 232 | gtaccaagcccagaag | 1-11-4 | GtaccaagcccaGAAG | 232_1 | 36280 |
| 233 | ttcctgatgaagagatg | 4-11-2 | TTCCtgatgaagagaTG | 233_1 | 36549 |
| 234 | tcctgatgaagagatg | 2-10-4 | TCctgatgaagaGATG | 234_1 | 36549 |
| 234 | tcctgatgaagagatg | 3-11-2 | TCCtgatgaagagaTG | 234_2 | 36549 |
| 235 | tgggagtagcatggc | 2-11-2 | TGggagtagcatgGC | 235_1 | 36911 |
| 235 | tgtgggagtagcatggc | 1-14-2 | TgtgggagtagcatgGC | 235_2 | 36911 |
| 236 | gtgggagtagcatggc | 1-13-2 | GtgggagtagcatgGC | 236_1 | 36911 |
| 237 | tgggagtagcatggc | 1-11-3 | TgggagtagcatGGC | 237_1 | 36911 |
| 238 | aaacatgctgaaccctg | 2-11-4 | AAacatgctgaacCCTG | 238_1 | 37254 |
| 239 | acaaacatgctgaaccct | 2-13-3 | ACaaacatgctgaacCCT | 239_1 | 37255 |
| 239 | acaaacatgctgaaccct | 1-14-3 | AcaaacatgctgaacCCT | 239_2 | 37255 |
| 239 | acaaacatgctgaaccct | 3-13-2 | ACAaacatgctgaaccCT | 239_3 | 37255 |
| 240 | cacaaacatgctgaaccc | 2-14-2 | CAcaaacatgctgaacCC | 240_1 | 37256 |
| 241 | cacaaacatgctgaacc | 2-12-3 | CAcaaacatgctgaACC | 241_1 | 37257 |
| 242 | tggacgcacaaacatgc | 1-12-4 | TggacgcacaaacATGC | 242_1 | 37263 |
| 243 | aattttacatactctggt | 243_1 | AATTttacatactctgGT | 4-12-2 | 11757 |
| 244 | aattttacatactctggtc | 244_1 | AAttttacatactctGGTC | 2-13-4 | 11756 |
| 245 | ttacatactctggtcaaa | 245_1 | TTacatactctggtCAAA | 2-12-4 | 11753 |
| 246 | ctttattataacttgaatctc | 246_1 | CTttattataactTgaAtCTC | 2-11- | 8681 |
| | | | | 1-2-1- | |
| | | | | 1-3 | |
| 246 | ctttattataacttgaatctc | 246_2 | CTttattataacttgaaTCTC | 2-17-4 | 8681 |

Motif sequences represent the contiguous sequence of nucleobases present in the
oligonucleotide.

Designs refer to the gapmer design, F-G-F′, where each number represents the number of consecutive modified nucleosides, e.g 2′ modified nucleosides (first number=5′ flank), followed by the number of DNA nucleosides (second number=gap region), followed by the number of modified nucleosides, e.g 2′ modified nucleosides (third number=3′ flank), optionally preceded by or followed by further repeated regions of DNA and LNA, which are not necessarily part of the contiguous sequence that is complementary to the target nucleic acid.

Oligonucleotide compounds represent specific designs of a motif sequence. Capital letters represent beta-D-oxy LNA nucleosides, lowercase letters represent DNA nucleosides, all LNA C are 5-methyl cytosine and 5-methyl DNA cytosines are presented by “e”, and all internucleoside linkages are phosphorothioate internucleoside linkages.

In all instances the F-G-F′ design may further include region D′ and/or D″ as described in the “Definitions” section under “Region D′ or D” in an oligonucleotide”. In some embodiments of the invention, the oligonucleotide has 1, 2 or 3 phosphodiester linked nucleoside units, such as DNA units, at the 5′ or 3′ end of the gapmer region. In some embodiments, the oligonucleotide consists of two 5′ phosphodiester linked DNA nucleosides followed by a F-G-F′ gapmer region as defined in the “Definitions” section. Oligonucleotides that contain phosphodiester linked DNA units at the 5′ or 3′ end are suitable for conjugation and may further comprise a conjugate moiety as described herein. For delivery to the liver ASGPR targeting moieties are particular advantageous as conjugate moieties.

For some embodiments of the invention, the oligonucleotide is selected from the group of oligonucleotide compounds with CMP-ID-NO: 27_1; 28_1; 29_1; 30_1; 31_1; 32_1; 33_1; 34_1; 35_1; 36_1; 37_1; 38_1; 39_1; 40_1; 41_1; 42_1; 43_1; 44_1; 45_1; 46_1; 47_1; 47_2; 47_3; 48_1; 48_2; 49_1; 50_1; 51_1; 52_1; 53_1; 54_1; 135_1; 114_1; 88_1; 208_1; 237_1; 243_1; 244_1; 245_1, 246_1 and 246_2 (see Table 9A and 9B).

In a preferred embodiment of the invention, the oligonucleotide is selected from the group of oligonucleotides compounds 243_1; 242_1; 245_1, 246_1 and 246_2 (see Table 9A and 9B).

In a preferred embodiment of the invention, the oligonucleotide is compound ID 243_1 (see Table 9A and 9B).

In a preferred embodiment of the invention, the oligonucleotide is compound ID 244_1 (see Table 9A and 9B).

In a preferred embodiment of the invention, the oligonucleotide is compound ID 245_1 (see Table 9A and 9B).

In a preferred embodiment of the invention, the oligonucleotide is compound ID 246_1 (see Table 9A and 9B).

In a preferred embodiment of the invention, the oligonucleotide is compound ID 246_2 (see Table 9A and 9B).

In some embodiments of the present invention, the antisense oligonucleotide comprises a contiguous nucleotide sequence of 12 to 22 nucleotides, such as of 15 to 20 nucleotides, with at least 90% complementarity, such as fully complementary, to the target nucleic acid of SEQ ID NO: 13.

In some embodiments, antisense oligonucleotide comprises a contiguous nucleotide sequence of 15 to 18 nucleotides, such as of 17 or 18 nucleotides, with at least 90% complementarity, such as fully complementary, to the target nucleic acid of SEQ ID NO: 16.

In some embodiments, antisense oligonucleotide comprises a contiguous nucleotide sequence of 15 to 19 nucleotides, such as of 18 or 19 nucleotides, with at least 90% complementarity, such as fully complementary, to the target nucleic acid of SEQ ID NO: 15.

In some embodiments, antisense oligonucleotide comprises a contiguous nucleotide sequence of 15 to 18 nucleotides, such as of 17 or 18 nucleotides, with at least 90% complementarity, such as fully complementary, to the target nucleic acid of SEQ ID NO: 14.

In some embodiments of the present invention, the antisense oligonucleotide of the combination of the present invention comprises a contiguous nucleotide sequence of 12 to 22 nucleotides, such as of 17 to 22 nucleotides, with at least 90% complementarity, such as fully complementary, to the target nucleic acid of SEQ ID NO: 5.

In some embodiments, the antisense oligonucleotide comprises a contiguous nucleotide sequence of 15 to 22 nucleotides, such as of 15 to 18 nucleotides, such as of 17 or 18 nucleotides with at least 90% complementarity, such as fully complementary, to the target nucleic acid selected from the following regions of SEQ ID NO: 1: 8681-8701 of SEQ ID NO: 1, 11753-11774 of SEQ ID NO: 1, such as to a region from nucleotides 8681-8701, 11757-11774, 11756-11774, or 11753-11770 of SEQ ID NO: 1.

In some embodiments, the contiguous nucleotide sequence comprises a sequence of nucleobases selected from the group consisting of SEQ ID NO: 243, 244, 245 and 246, or at least 14 contiguous nucleotides thereof.

In some embodiments, the antisense oligonucleotide or contiguous nucleotide sequence thereof, comprises or consists of 10 to 30 nucleotides in length, such as from 12 to 25, such as 11 to 22, such as from 12 to 20, such as from 14 to 18 or 14 to 16 contiguous nucleotides in length.

In some embodiments, the antisense oligonucleotide or contiguous nucleotide sequence thereof comprises or consists of 22 or less nucleotides, such as 20 or less nucleotides, such as 18 or less nucleotides, such as 14, 15, 16 or 17 nucleotides. It is to be understood that any range given herein includes the range endpoints. Accordingly, if an oligonucleotide is said to include from 10 to 30 nucleotides, both 10 and 30 nucleotides are included.

In some embodiments, the contiguous nucleotide sequence comprises or consists of 10, 11, 12, 13, 14, 15, 16, 17, 18, 19, 20, 21 or 22 contiguous nucleotides in length.

In some embodiments, the antisense oligonucleotide or contiguous nucleotide sequence thereof comprises or consists of a sequence selected from SEQ ID NO: 243, 244, 245 and 246.

The antisense oligonucleotides are such as antisense oligonucleotides of 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 13.

The antisense oligonucleotides useful in the invention are such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 16.

The antisense oligonucleotides useful in the invention are such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 15.

The antisense oligonucleotides useful in the invention are such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 14.

The antisense oligonucleotides useful in the invention are such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 5.

In advantageous embodiments, the antisense oligonucleotide comprises one or more sugar modified nucleosides, such as one or more 2′ sugar modified nucleosides, such as one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In some embodiments, the contiguous nucleotide sequence comprises LNA nucleosides.

In some embodiments, of the oligonucleotide, all LNA nucleosides are beta-D-oxy LNA nucleosides.

In some embodiments, the contiguous nucleotide sequence comprises LNA nucleosides and DNA nucleosides.

In some embodiments, the contiguous nucleotide sequence comprises 2′-O-methoxyethyl (2′MOE) nucleosides.

In some embodiments, the contiguous nucleotide sequence comprises 2′-O-methoxyethyl (2′MOE) nucleosides and DNA nucleosides.

Advantageously, the 3′ most nucleoside of the antisense oligonucleotide, or contiguous nucleotide sequence thereof is a 2′sugar modified nucleoside.

Advantageously, the antisense oligonucleotide comprises at least one modified internucleoside linkage, such as phosphorothioate or phosphorodithioate.

In some embodiments, the at least one internucleoside linkage in the contiguous nucleotide sequence is a phosphorothioate internucleoside linkages.

In some embodiments, at least one internucleoside linkage in the contiguous nucleotide sequence is a phosphorodithioate internucleoside linkages.

In some embodiments, at least one internucleoside linkage in the contiguous nucleotide sequence is a phosphodiester internucleoside linkages.

In some embodiments, all the internucleoside linkages within the contiguous nucleotide sequence are phosphorothioate internucleoside linkages.

In some embodiments, at least 75% the internucleoside linkages within the antisense oligonucleotide, or contiguous nucleotide sequence thereof, are phosphorothioate internucleoside linkages.

In some embodiments, all the internucleoside linkages within the antisense oligonucleotide, or contiguous nucleotide sequence thereof, are phosphorothioate internucleoside linkages.

In an advantageous embodiment of the invention the antisense oligonucleotide is capable of recruiting RNase H, such as RNase H1. In some embodiments, the antisense oligonucleotide, or the contiguous nucleotide sequence thereof is a gapmer.

In some embodiments, the antisense oligonucleotide, or contiguous nucleotide sequence thereof, consists or comprises a gapmer of formula 5′-F-G-F′-3′.

In some embodiments, region G consists of 6-16 DNA nucleoside, such as 11 to 16 DNA nucleosides. In some embodiments, region F comprises 2 to 4 DNA nucleosides and/or region F′ comprises DNA 2 to 6 nucleosides.

In some embodiments, region F and F′ each comprise at least one LNA nucleoside.

In some embodiments, the oligonucleotide of the present invention is a LNA gapmer with uniform flanks. For example, the LNA gapmer with uniform flanks may have a design selected from the following designs: 1-12-3, 4-12-2, 2-17-4, 2-13-4 and 2-12-4. Table 9B lists preferred designs for each motif sequence.

In some embodiments, of the invention, the LNA gapmer is an alternating flank LNA gapmer. In some embodiments, the alternating flank LNA gapmer comprises at least one alternating flank (such as flank F′). In some embodiments, the alternating flank LNA gapmer comprises one alternating flank (such as flank F′) and one uniform flank (such as flank F). For example, the LNA gapmer with one alternating F′ flank may have the following design: 2-11-1-2-1-1-3.

The invention provides the following oligonucleotide compounds (Table 9B and 10):

TABLE 9B
list of suitable oligonucleotide motif sequences for use in the invention (indicated by
SEQ ID NO), designs of these, as well as specific oligonucleotide compounds for use in
the invention (indicated by CMP ID NO) designed based on the motif sequence.

| | | position on | | | | |
|---|---|---|---|---|---|---|
| SEQ ID | | SEQ ID NO: 1 | | | CMP ID | Oligonucleotide |
| NO | Motif sequence | Start | end | Design | NO | Compound |
| 243 | AATTTTACATACTCTG | 11757 | 11774 | 4-12-2 | 243_1 | AATTttacatactctgGT |
| | GT | | | | | |
| 244 | AATTTTACATACTCTG | 11756 | 11774 | 2-13-4 | 244_1 | AAttttacatactctGGTC |
| | GTC | | | | | |
| 245 | TTACATACTCTGGTC | 11753 | 11770 | 2-12-4 | 245_1 | TTacatactctggtCAAA |
| | AAA | | | | | |
| 246 | CTTTATTATAACTTGA | 8681 | 8701 | 2-11-1- | 246_1 | CTttattataactTgaAtCT |
| | ATCTC | | | 2-1-1-3 | | C |
| 246 | CTTTATTATAACTTGA | 8681 | 8701 | 2-17-4 | 246_2 | CTttattataacttgaaTCT |
| | ATCTC | | | | | C |

TABLE 10
Compound Table (Exemplary antisense oligonucleotides for use in the present
invention) - HELM Annotation Format

| SEQ | Compound | | Comprised |
|---|---|---|---|
| ID | ID | HELM Annotation | by conjugate |
| Number | Number # | Written 5′-3′ | shown in FIG. |
| 243 | 243_1 | [LR](A)[sP].[LR](A)[sP].[LR](T)[sP].[LR](T)[sP].[dR] | 1 |
| | | (T)[sP].[dR](T)[sP].[dR](A)[sP].[dR](C)[sP].[dR](A) | |
| | | [sP].[dR](T)[sP].[dR](A)[sP].[dR](C)[sP].[dR](T)[sP]. | |
| | | [dR](C)[sP].[dR](T)[sP].[dR](G)[sP].[LR](G)[sP].[LR](T) | |
| 244 | 244_1 | [LR](A)[sP].[LR](A)[sP].[dR](T)[sP].[dR](T)[sP].[dR] | 2 |
| | | (T)[sP].[dR](T)[sP].[dR](A)[sP].[dR](C)[sP].[dR](A) | |
| | | [sP].[dR](T)[sP].[dR](A)[sP].[dR](C)[sP].[dR](T)[sP]. | |
| | | [dR](C)[sP].[dR](T)[sP].[LR](G)[sP].[LR](G)[sP].[LR](T) | |
| | | [sP].[LR]([5meC]) | |
| 245 | 245_1 | [LR](T)[sP].[LR](T)[sP].[dR](A)[sP].[dR](C)[sP].[dR] | 4 |
| | | (A)[sP].[dR](T)[sP].[dR](A)[sP].[dR](C)[sP].[dR](T) | |
| | | [sP].[dR](C)[sP].[dR](T)[sP].[dR](G)[sP].[dR](G)[sP]. | |
| | | [dR](T)[sP].[LR]([5meC])[sP].[LR](A)[sP].[LR](A)[sP]. | |
| | | [LR](A) | |
| 246 | 246_1 | [LR]([5meC])[sP].[LR](T)[sP].[dR](T)[sP].[dR](T)[sP]. | 3 |
| | | [dR](A)[sP].[dR](T)[sP].[dR](T)[sP].[dR](A)[sP].[dR] | |
| | | (T)[sP].[dR](A)[sP].[dR](A)[sP].[dR](C)[sP].[dR](T) | |
| | | [sP].[LR](T)[sP].[dR](G)[P].[dR](A)[sP].[LR](A)[sP]. | |
| | | [dR](T)[sP].[LR]([5meC])[sP].[LR](T)[sP].[LR]([5meC]) | |
| 246 | 246_2 | [LR]([5meC])[sP].[LR](T)[sP].[dR](T)[sP].[dR](T)[sP]. | |
| | | [dR](A)[sP].[dR](T)[sP].[dR](T)[P].[dR](A)[sP].[dR] | |
| | | (T)[sP].[dR](A)[sP].[dR](A)[sP].[dR](C)[sP].[dR](T) | |
| | | [sP].[dR](T)[sP].[dR](G)[sP].[dR](A)[sP].[dR](A)[sP]. | |
| | | [LR](T)[sP].[LR]([5meC])[sP].[LR](T)[sP].[LR]([5meC]) | |

### Helm Annotation Key:

[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC]) is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR](C) is a DNA cytosine nucleoside,
[sP] is a phosphorothioate internucleoside linkage,
P is a phosphodiester internucleoside linkage.

The heading “Oligonucleotide compound” in the table 9A and 9B represents specific designs of a motif sequence. Capital letters are beta-D-oxy LNA nucleosides, lowercase letters are DNA nucleosides, all LNA C are 5-methyl cytosine, all internucleoside linkages are phosphorothioate internucleoside linkages. The heading “Designs” refers to the gapmer design, F-G-F′. In classic gapmer design, i.e.gapmers with uniform flanks (e.g. 4-12-2), all the nucleotides in the flanks (F and F′) are constituted of the same type of 2′-sugar modified nucleoside, e.g. LNA, cET, or MOE, and a stretch of DNA in the middle forming the gap (G). In gapmers with alternating flank designs, the flanks of oligonucleotide are annotated as a series of integers, representing a number of beta-D-oxy LNA nucleosides (L) followed by a number of DNA nucleosides (D). For example, a flank F′ with a 1-2-1-1-3 motif represents LDDLDLLL (see CMP ID NO 325_1). Both flanks have a beta-D-oxy LNA nucleoside at the 5′ and 3′ terminal. The gap region (G), which is constituted of a number of DNA nucleosides is located between the flanks.

For some embodiments of the invention, the oligonucleotide is selected from the group of oligonucleotide compounds consisting of CMP-ID-NO: 243_1, 244_1, 245_1, 246_1 and 246_2 (see Table 9B).

In all instances, the F-G-F′ design may further include region D′ and/or D″ as described in the “Definitions” section under “Region D′ or D” in an oligonucleotide”. In some embodiments of the invention, the oligonucleotide has 1, 2 or 3 phosphodiester linked nucleoside units, such as DNA units, at the 5′ or 3′ end, such as at the 5′ end, of the gapmer region. In some embodiments of the invention, the oligonucleotide consists of two 5′ phosphodiester linked DNA nucleosides followed by a F-G-F′ gapmer region as defined above. Oligonucleotides that contain phosphodiester linked DNA units at the 5′ or 3′ end are suitable for conjugation and may further comprise a conjugate moiety as described herein. For delivery to the liver ASGPR targeting moieties are particular advantageous as conjugate moieties, see the Conjugate section for further details.

### FUBP1 Inhibitors

In one aspect, the second category of compound in the combination of the invention is an inhibitor targeting FUBP1. Such an inhibitor can be selected from the group consisting of, for example, small molecules, single stranded antisense oligonucleotide; siRNA molecule; or shRNA molecule.

Without being bound by theory, it is believed that FUBP1 is involved in the stabilization of the cccDNA in the cell nucleus, and by preventing the binding of FUBP1 to DNA, in particular cccDNA, the cccDNA is destabilised and becomes prone to degradation. One embodiment of the invention therefore comprises a FUBP1 inhibitor which interacts with the DNA binding domain of FUBP1 protein, and prevents or reduces binding to cccDNA.

### Small Molecules Inhibiting FUBP1

Small molecules inhibiting FUBP1 have been identified in relation to FUBP1's role in cancer, where the small molecule inhibits the DNA binding activity of FUBP1, in particular the binding to the FUSE element on a single stranded DNA. In the present invention, FUBP1 inhibitors are envisioned as useful in treating HBV. In particular targeting of such small molecule compounds, e.g. via conjugation or formulation, to the liver may be beneficial in the treatment of HBV.

Huth et al 2004 J Med. Chem Vol 47 p. 4851-4857 discloses a series of benzoyl anthranilic acid compounds capable of binding to a four tandem K homology (KH) repeat of FUBP1. All the compounds disclosed in Huth et al 2004 are hereby incorporated by reference. In particular the compounds of formula I, II or III shown below were found to be efficient in inhibiting FUBP1 DNA binding activity.

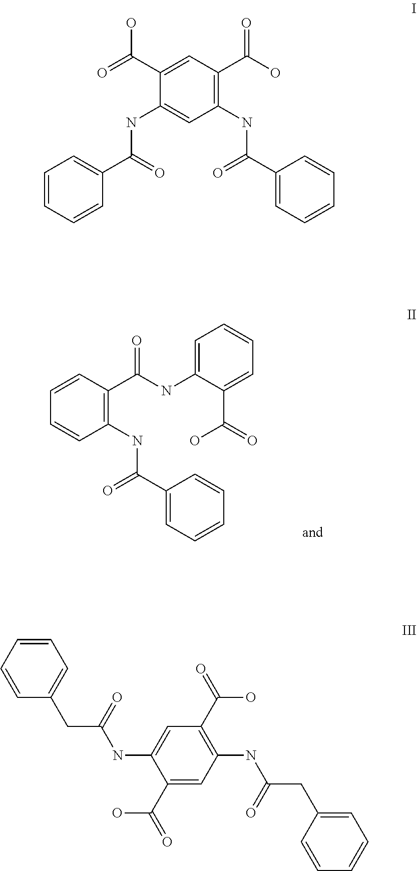 [SMILES: O=C(Nc1cc(NC(=O)c2ccccc2)c(C(=O)O)cc1C(=O)O)c1ccccc1 ; O=C(Nc1ccccc1C(=O)Nc1ccccc1C(=O)O)c1ccccc1 ; O=C(Cc1ccccc1)Nc1cc(C(=O)O)c(NC(=O)Cc2ccccc2)cc1C(=O)O]

One embodiment of the present invention comprises a compound of formula I, II or III for use in treatment and/or prevention of Hepatitis B virus (HBV) infection.

Hauck et al 2016 Bioorganic & Medicinal Chemistry Vol 24 p. 5717-5729 describes an additional series of compounds with high FUBP1 inhibitory potential (see table 2, hereby incorporated by reference). In particular, the following compounds of formula IV were effective in inhibiting FUBP1 activity

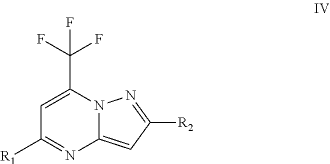

wherein R¹ is selected from

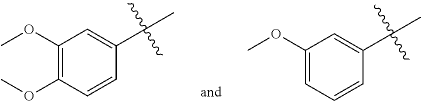

and
R2 is selected from

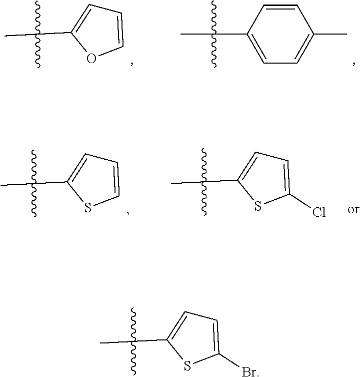

Specifically the compounds of formula V, VI and VII were shown to have IC50 values below 15 μM.

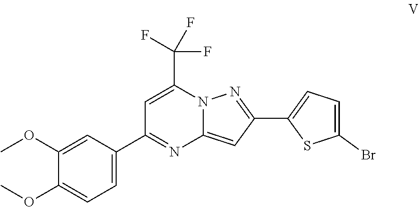 [SMILES: COc1ccc(-c2cc(C(F)(F)F)n3nc(-c4ccc(Br)s4)cc3n2)cc1OC]

### 2-(5-Bromothiophen-2-yl)-5-(3,4-dimethoxyphenyl)-7-(trifluoromethyl) pyrazolo[1,5-a]pyrimidine

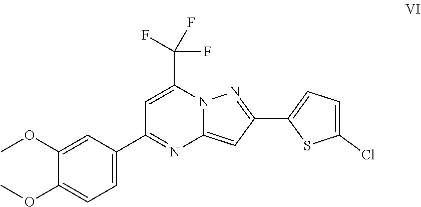 [SMILES: COc1ccc(-c2cc(C(F)(F)F)n3nc(-c4ccc(Cl)s4)cc3n2)cc1OC]

### 2-(5-Chlorothiophen-2-yl)-5-(3,4-dimethoxyphenyl)-7-(trifluoromethyl) pyrazolo[1,5-a]pyrimidine

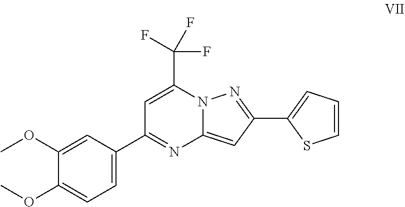 [SMILES: COc1ccc(-c2cc(C(F)(F)F)n3nc(-c4cccs4)cc3n2)cc1OC]

### 5-(3,4-Dimethoxyphenyl)-2-(thiophen-2-yl)-7-(trifluoromethyl) pyrazolo[1,5-a]pyrimidine

One embodiment of the present invention comprises a compound of formula IV for use in treatment and/or prevention of Hepatitis B virus (HBV) infection.

One embodiment of the present invention comprises a compound of formula V, VI or VI for use in treatment and/or prevention of Hepatitis B virus (HBV) infection.

The S-adenosyl-L-methionine (SAM) competitive inhibitor, GSK343 (formula VIII), is currently in preclinical development for osteosarcoma. It has been shown that GSK343 inhibits FUBP1 expression in osteosarcoma cells (Xiong et al 2016 Int J Onc vol 49 p 623).

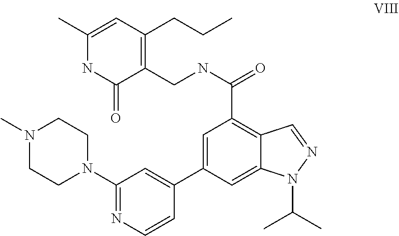 [SMILES: CCCc1cc(C)[nH]c(=O)c1CNC(=O)c1cc(-c2ccnc(N3CCN(C)CC3)c2)cc2c1cnn2C(C)C]

One embodiment of the present invention comprises a compound of formula VII for use in treatment and/or prevention of Hepatitis B virus (HBV) infection.

The FDA-approved cancer drugs camptothecin (CPT, formula IX) and its derivative SN-38 (7-ethyl-10-hydroxycamptothecin, formula X), which are Topoisomerase I (TOP1) inhibitors, were recently shown to inhibit FUBP1 activity as well by preventing the FUBP1/FUSE interaction (Hosseini et al 2017 Biochemical Pharmacology Vol 146 p. 53-62).

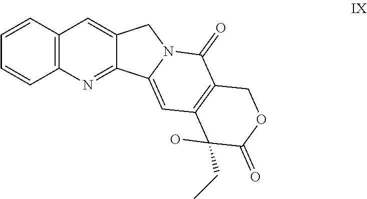 [SMILES: CC[C@@]1(O)C(=O)OCc2c1cc1n(c2=O)Cc2cc3ccccc3nc2-1]

### Camptothecin ((+)-4 (S)-Ethyl-4-hydroxy-3,4,12, 14-tetrahydro-1H-pyrano [3′,4′: 6,7]indolizino [1,2-b]quinoline-3,14-dione)

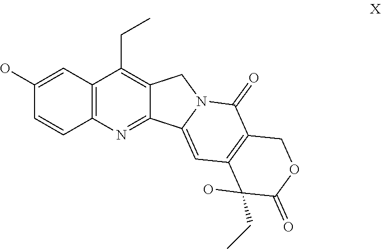 [SMILES: CCc1c2c(nc3ccc(O)cc13)-c1cc3c(c(=O)n1C2)COC(=O)[C@]3(O)CC]

### SN-38 (4 (S), 11-Diethyl-4,9-dihydroxy-3,4,12, 14-tetrahydro-1H-pyrano [3′,4′: 6,7]indolizino [1,2-b]quinoline-3,14-dione 7-Ethyl-10-hydroxycamptothecin)

One embodiment of the present invention comprises a compound of formula IX or X for use in treatment and/or prevention of Hepatitis B virus (HBV) infection.

Tringali et al 2012 Journal of Pharmacy and Pharmacology Vol 64, p. 360-365 describes the pharmacokinetic profile SN-38 conjugated to hyaluronic acid (HA-SN-38, formula XI) and shows an increased distribution to the liver.

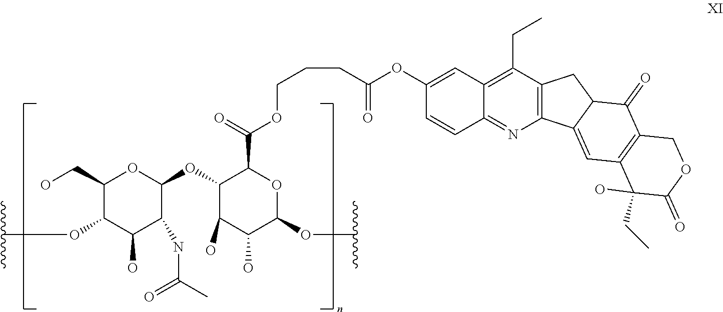

One embodiment of the present invention comprises a compound of formula XI for use in treatment and/or prevention of Hepatitis B virus (HBV) infection.

Various lipid conjugates of SN-38 also exist in the literature. WO2006/082053 for example describes the molecule of formula XII

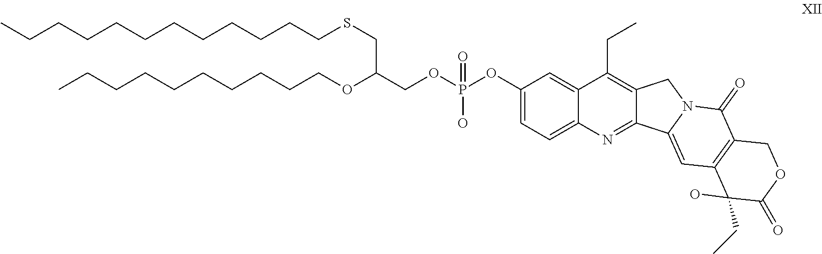 [SMILES: CCCCCCCCCCCCSCC(COP(=O)(O)Oc1ccc2nc3c(c(CC)c2c1)Cn1c-3cc2c(c1=O)COC(=O)[C@]2(O)CC)OCCCCCCCCCC]

CN105777770 describes a palmitate conjugated SN-38 shown in formula XIII below.

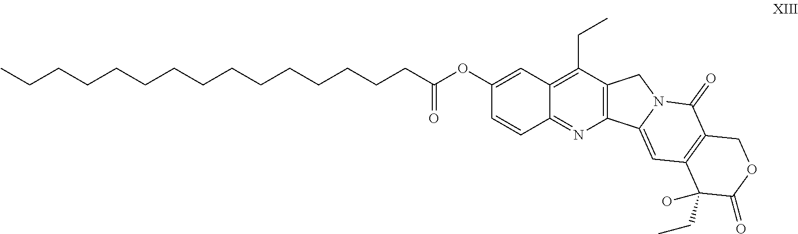 [SMILES: CCCCCCCCCCCCCCCC(=O)Oc1ccc2nc3c(c(CC)c2c1)Cn1c-3cc2c(c1=O)COC(=O)[C@]2(O)CC]

One embodiment of the present invention comprises a compound of formula XII or XIII for use in 5 treatment and/or prevention of Hepatitis B virus (HBV) infection.

In a further aspect of the invention the FUBP1 inhibitors, for example for use in treatment and/or prevention of Hepatitis B virus (HBV) infection, can be targeted directly to the liver by covalently attaching them to a conjugate moiety capable of binding to the asialoglycoprotein receptor (ASGPr), such as divalent or trivalent GalNAc cluster.

siRNA Targeting FUBP1

TABLE 11
Human FUBP1 sequences targeted by individual FUBP1 siRNA molecules

| SEQ | | Position | | | |
|---|---|---|---|---|---|
| ID | | on SEQ | | Individual | Dharmacon |
| NO: | FUBP1 target sequence | ID NO: 1 | Exon | SIRNA | catalog No. |
| 21 | GACAAACCUCUUAGGAUUA | 14200- | 9 | FU2 | J-011548-06 |
| | | 14218 | | | |
| 22 | GAGAAGUUCGGAAUGAGUA | 14413- | 10 | FU4 | J-011548-08 |
| | | 14431 | | | |
| 23 | GAAAGGAUAGCACAAAUAA | 14966- | 12 | FU1 | J-011548-05 |
| | | 14984 | | | |
| 24 | AAUAAGAAGUGGACAAUAC | 30344- | 20 | FU3 | J-011548-07 |
| | | 30362 | | | |

The pool of siRNA (ON-TARGETplus SMART pool siRNA Cat. No. L-011548-00-0005, Dharmacon) contains the four individual siRNA molecules listed in table 11 and are available.

### Oligonucleotides Targeting FUBP1

In the present section, the term “oligonucleotide” is to be understood as “oligonucleotide targeting FUBP1”.

Nucleic acid molecules (or oligonucleotides) are potentially excellent FUBP1 inhibitors since they can target the FUBP1 transcript and promote its degradation either via the RNA interference pathway or via RNaseH cleavage. Alternatively, nucleic acid molecules such as aptamers can also act as inhibitors of the DNA binding site of FUBP1 in line with the small molecules described above.

In one aspect of the present invention, the combination comprises a nucleic acid molecule for use in treatment and/or prevention of Hepatitis B virus (HBV) infection. Such nucleic acid molecules can be selected from the group consisting of single stranded antisense oligonucleotide; siRNA molecule; or shRNA molecule.

The nucleic acid molecules useful in the present invention are capable of inhibiting the expression of FUBP1 in vitro and in vivo. The inhibition is achieved by hybridizing an oligonucleotide to a target nucleic acid encoding FUBP1.

The target nucleic acid may be a mammalian FUBP1 sequence, such as a sequence selected from the group consisting of SEQ ID NO: 247 to 266. It is advantageous if the mammalian FUBP1 sequence is selected from the group consisting of SEQ ID NO: 247, 248, 249, 250, 251, 252, 253, and 254.

In some embodiments, the nucleic acid molecule useful in the invention is capable of modulating the expression of FUBP1 by inhibiting or down-regulating it. Preferably, such modulation produces an inhibition of expression of at least 40% compared to the normal expression level of the target, more preferably at least 50%, 60%, 70%, 80%, 90%, 95% or 98% inhibition compared to the normal expression level of the target. In some embodiments, the nucleic acid molecule useful in the invention is capable of inhibiting expression levels of FUBP1 mRNA by at least 65%-98%, such as 70% to 95%, in vitro using HepG2-NTCP cells or HBV infected primary human hepatocytes, this range of target reduction is advantageous in terms of selecting nucleic acid molecules with good correlation to the cccDNA reduction. In some embodiments, oligonucleotides useful in the invention may be capable of inhibiting expression levels of FUBP1 protein by at least 50% in vitro using HepG2-NTCP cells or HBV infected primary human hepatocytes. The materials and Method section and the Examples herein provide assays which may be used to measure target RNA inhibition in HepG2-NTCP cells or HBV infected primary human hepatocytes as well as cccDNA. The target modulation is triggered by the hybridization between a contiguous nucleotide sequence of the oligonucleotide, such as the guide strand of a siRNA or gapmer region of an antisense oligonucleotide, and the target nucleic acids. In some embodiments, the oligonucleotide useful in the invention comprises mismatches between the oligonucleotide or the contiguous nucleotide sequence and one or both of the target nucleic acids. Despite mismatches hybridization to the target nucleic acid may still be sufficient to show a desired modulation of FUBP1 expression. Reduced binding affinity resulting from mismatches may advantageously be compensated by increased length of the oligonucleotide and/or an increased number of modified nucleosides capable of increasing the binding affinity to the target within the oligonucleotide sequence. Advantageously, the oligonucleotides useful in the present invention contain modified nucleosides capable of increasing the binding affinity, such as 2′ sugar modified nucleosides, including LNA.

An aspect of the present invention relates a combination comprising a nucleic acid molecule of 12 to 60 nucleotides in length, which comprises a contiguous nucleotide sequence of 12 to 30 nucleotides in length which is capable of inhibiting the expression of FUBP1.

In some embodiments, the nucleic acid molecule comprises a contiguous sequence which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

In one embodiment, the nucleic acid molecule of the combination of the invention, or contiguous nucleotide sequence thereof, is fully complementary (100% complementary) to a region of the target nucleic acids, or in some embodiments may comprise one or two mismatches between the oligonucleotide and the target nucleic acids.

In some embodiments, the nucleic acid molecule comprises a contiguous nucleotide sequence of 12 to 30 nucleotides in length with at least 95% complementary, such as fully (or 100%) complementary, to a target nucleic acid region present in SEQ ID NO: 247, SEQ ID NO: 248, SEQ ID NO: 249 and/or SEQ ID NO:250.

In some embodiments, the nucleic acid molecule or the contiguous nucleotide sequence is at least 93% complementarity, such as fully (or 100%) complementary, to the target nucleic acid of SEQ ID NO: 247, SEQ ID NO: 248, SEQ ID NO: 249, SEQ ID NO: 250, SEQ ID NO: 251, SEQ ID NO: 252, SEQ ID NO; 253 and/or SEQ ID NO; 254.

In some embodiments the nucleic acid molecule or the contiguous nucleotide sequence is at least 95% complementarity, such as fully (or 100%) complementary, to the target nucleic acid of SEQ ID NO: 247 and SEQ ID NO: 251.

In some embodiments the nucleic acid molecule or the contiguous nucleotide sequence is at least 95% complementarity, such as fully (or 100%) complementary, to the target nucleic acid of SEQ ID NO: 247, SEQ ID NO: 251 and SEQ ID NO: 255.

In some embodiments the nucleic acid molecule or the contiguous nucleotide sequence is 100% complementary to position 14200-14218 on SEQ ID NO: 247.

In some embodiments the nucleic acid molecule or the contiguous nucleotide sequence is 100% complementary to position 14413-14431 on SEQ ID NO: 247.

In some embodiments the nucleic acid molecule or the contiguous nucleotide sequence is 100% complementary to position 14966-14984 on SEQ ID NO: 247.

In some embodiments the nucleic acid molecule or the contiguous nucleotide sequence is 100% complementary to position 30344-30362 on SEQ ID NO: 247

In some embodiments, the nucleic acid molecule comprises or consists of 12 to 60 nucleotides in length, such as from 13 to 50, such as 14 to 35, such as from 15 to 30 such as from 16 to 22 nucleotides in length.

In some embodiments, the contiguous nucleotide sequence of the acid molecule which is complementary to the target nucleic acids comprises or consists of 12 to 30, such as from 14 to 25, such as from 16 to 23, such as from 18 to 22, contiguous nucleotides in length.

In some embodiments, the contiguous nucleotide sequence of the siRNA or shRNA which is complementary to the target nucleic acids comprises or consists of 18 to 28, such as from 19 to 26, such as from 20 to 24, such as from 21 to 23, contiguous nucleotides in length.

In some embodiments, the contiguous nucleotide sequence of the antisense oligonucleotide which is complementary to the target nucleic acids comprises or consists of 12 to 22, such as from 14 to 20, such as from 16 to 20, such as from 15 to 18, such as from 16 to 18, such as from 16 to 17 contiguous nucleotides in length.

In some embodiments, the oligonucleotide or contiguous nucleotide sequence comprises or consists of 10 to 30 nucleotides in length with at least 90% identity, preferably 100% identity, to a sequence selected from the group consisting of SEQ ID NO: 275 to 330 (see motif sequences listed in table 12A). In a particular embodiment, the oligonucleotide or contiguous nucleotide sequence is selected from SEQ ID NO: 325; 326; 327; 328; 329 and 330.

TABLE 12A
list of oligonucleotide motif sequences (indicated by SEQ ID NO), designs of these,
as well as specific oligonucleotide compounds (indicated by CMP ID NO) designed based
on the motif sequence.

| | | | | | Start |
|---|---|---|---|---|---|
| | | | | | position |
| SEQ | | | | CMP | on SEQ |
| ID | | | Oligonucleotide | ID | ID NO: |
| NO | Motif sequence | Design | Compound | NO | 247 |
| 275 | ataaccatagtcatttga | 3-12-3 | ATAaccatagtcattTGA | 275_1 | 9138 |
| 276 | cccataaccatagtcat | 3-12-2 | CCCataaccatagtcAT | 276_1 | 9142 |
| 277 | gagccatctacacataaa | 2-12-4 | GAgccatctacacaTAAA | 277_1 | 10016 |
| 278 | caagagccatctacacat | 3-13-2 | CAAgagccatctacacAT | 278_1 | 10019 |
| 279 | tgtccatttaagaatcca | 4-12-2 | TGTCcatttaagaatcCA | 279_1 | 10144 |
| 280 | ttgtgtccatttaagaat | 4-12-2 | TTGTgtccatttaagaAT | 280_1 | 10147 |
| 281 | tacctttgctgctgat | 3-11-2 | TACctttgctgctgAT | 281_1 | 11472 |
| 282 | gactatacctttgctgc | 2-13-2 | GActatacctttgctGC | 282_1 | 11476 |
| 283 | actttgtattcttctgtcat | 1-16-3 | ActttgtattcttctgtCAT | 283_1 | 12020 |
| 284 | actttgtattcttctgtc | 2-14-2 | ACtttgtattcttctgTC | 284_1 | 12022 |
| 285 | gattcaggtgttccagtta | 1-16-2 | GattcaggtgttccagtTA | 285_1 | 12393 |
| 286 | ttcaaacttactggaca | 3-11-3 | TTCaaacttactggACA | 286_1 | 12412 |
| 287 | tacatctatcccttcat | 2-12-3 | TAcatctatcccttCAT | 287_1 | 14452 |
| 288 | ttacatctatcccttc | 2-10-4 | TTacatctatccCTTC | 288_1 | 14454 |
| 289 | cttcctattacaatgccaac | 1-17-2 | CttcctattacaatgccaAC | 289_1 | 14776 |
| 290 | atttcttcctattacaatg | 2-14-3 | ATttcttcctattacaATG | 290_1 | 14781 |
| 291 | ccatttcttcctattacaa | 3-14-2 | CCAtttcttcctattacAA | 291_1 | 14783 |
| 292 | gtttaaaatacattgcc | 2-11-4 | GTttaaaatacatTGCC | 292_1 | 15537 |
| 293 | gagtttaaaatacattgcc | 2-14-3 | GAgtttaaaatacattGCC | 293_1 | 15537 |
| 294 | gctttttatggtttcacc | 1-15-2 | GctttttatggtttcaCC | 294_1 | 16183 |
| 295 | atgctttttatggtttcacc | 1-17-2 | AtgctttttatggtttcaCC | 295_1 | 16183 |
| 296 | ataatcaacctgtccagct | 1-16-2 | AtaatcaacctgtccagCT | 296_1 | 23804 |
| 297 | ataatcaacctgtccagc | 1-15-2 | AtaatcaacctgtccaGC | 297_1 | 23805 |
| 298 | ataatcaacctgtccag | 1-12-4 | AtaatcaacctgtCCAG | 298_1 | 23806 |
| 299 | tataatcaacctgtccag | 2-13-3 | TAtaatcaacctgtcCAG | 299_1 | 23806 |
| 300 | gtataatcaacctgtccag | 1-15-3 | GtataatcaacctgtcCAG | 300_1 | 23806 |
| 301 | ataatcaacctgtcca | 1-11-4 | AtaatcaacctgTCCA | 301_1 | 23807 |
| 302 | tataatcaacctgtcca | 1-12-4 | TataatcaacctgTCCA | 302_1 | 23807 |
| 303 | gtataatcaacctgtcca | 1-14-3 | GtataatcaacctgtCCA | 303_1 | 23807 |
| 304 | gtataatcaacctgtcc | 3-12-2 | GTAtaatcaacctgtCC | 304_1 | 23808 |
| 305 | tataatcaacctgtcc | 2-10-4 | TAtaatcaacctGTCC | 305_1 | 23808 |
| 306 | cccattttcttgtagta | 2-13-2 | CCcattttcttgtagTA | 306_1 | 23841 |
| 307 | acccattttcttgtagta | 1-15-2 | AcccattttcttgtagTA | 307_1 | 23841 |
| 308 | tacccattttcttgtagta | 1-16-2 | TacccattttcttgtagTA | 308_1 | 23841 |
| 309 | atacccattttcttgtagta | 1-17-2 | AtacccattttcttgtagTA | 309_1 | 23841 |
| 310 | atacccattttcttgtag | 1-14-3 | AtacccattttcttgTAG | 310_1 | 23843 |
| 311 | catacccattttcttgtag | 2-15-2 | CAtacccattttcttgtAG | 311_1 | 23843 |
| 312 | catacccattttcttgta | 2-14-2 | CAtacccattttcttgTA | 312_1 | 23844 |
| 313 | tggagctaattcaggagt | 2-14-2 | TGgagctaattcaggaGT | 313_1 | 30264 |
| 314 | aaatggagctaattcaggag | 3-14-3 | AAAtggagctaattcagGAG | 314_1 | 30265 |
| 315 | ttgtccacttcttattatt | 1-14-4 | TtgtccacttcttatTATT | 315_1 | 30341 |
| 316 | ccccacacaatgaagcaa | 2-14-2 | CCccacacaatgaagcAA | 316_1 | 30368 |
| 317 | ttcatcaagtcgtctgcat | 1-16-2 | TtcatcaagtcgtctgcAT | 317_1 | 30412 |
| 318 | gatcttcatcaagtcgtc | 1-15-2 | GatcttcatcaagtcgTC | 318_1 | 30417 |
| 319 | atattaacctcctatcagt | 1-15-3 | AtattaacctcctatcAGT | 319_1 | 30511 |
| 320 | aatattaacctcctatcag | 3-13-3 | AATattaacctcctatCAG | 320_1 | 30512 |
| 321 | atttatatcacaaagcatc | 2-13-4 | ATttatatcacaaagCATC | 321_1 | 30616 |
| 322 | aagtacatttatatcaca | 4-12-2 | AAGTacatttatatcaCA | 322_1 | 30623 |
| 323 | catttattgtaaagcacaaa | 2-14-4 | CAtttattgtaaagcaCAAA | 323_1 | 30675 |
| 324 | atcatttattgtaaagca | 2-12-4 | ATcatttattgtaaAGCA | 324_1 | 30679 |
| 325 | cttatgctttttatggt | 3-2-1-9- | CTTatGctttttatgGT | 325_1 | 16189 |
| | | 2 | | | |
| 325 | cttatgctttttatggt | 3-1-1- | CTTaTgctttttatgGT | 325_2 | 16189 |
| | | 10-2 | | | |
| 326 | cttatgctttttatggtt | 2-1-2- | CTtATgctttttatgGTT | 326_1 | 16188 |
| | | 10-3 | | | |
| 326 | cttatgctttttatggtt | 2-1-1- | CTtAtgctttttatgGTT | 326_2 | 16188 |
| | | 11-3 | | | |
| 326 | cttatgctttttatggtt | 2-1-1- | CTtAtgctttttatGgTT | 326_3 | 16188 |
| | | 10-1-1-2 | | | |
| 326 | cttatgctttttatggtt | 2-1-1- | CTtAtgctttttatGGTT | 326_4 | 16188 |
| | | 10-4 | | | |
| 327 | gctttttatggtttcac | 1-3-1-7- | GcttTttatggtTtCAC | 327_1 | 16184 |
| | | 1-1-3 | | | |
| 328 | tatgctttttatggtttc | 3-2-1-9- | TATgcTttttatggtTTC | 328_1 | 16186 |
| | | 3 | | | |
| 329 | accaattttcatttctac | 1-1-3-9- | AcCAAttttcatttCtAC | 329_1 | 30536 |
| | | 1-1-2 | | | |
| 330 | ccccataaccatagtc | 1-12-3 | CcccataaccataGTC | 330_1 | 9141 |

It is understood that the contiguous nucleobase sequences (motif sequence) can be modified to for example increase nuclease resistance and/or binding affinity to the target nucleic acid.

The pattern in which the modified nucleosides (such as high affinity modified nucleosides) are incorporated into the oligonucleotide sequence is generally termed oligonucleotide design. The oligonucleotides of the combination of the invention are designed with modified nucleosides and RNA nucleosides (in particular for siRNA and shRNA molecules) or DNA nucleosides (in particular for single stranded antisense oligonucleotides). Advantageously, high affinity modified nucleosides are used.

In an embodiment, the oligonucleotide comprises at least 1 modified nucleoside, such as at least 2, at least 3, at least 4, at least 5, at least 6, at least 7, at least 8 modified nucleosides. In an embodiment the oligonucleotide comprises from 1 to 8 modified nucleosides, such as from 2 to 7 modified nucleosides, such as from 3 to 6 modified nucleosides, such as from 4 to 6 modified nucleosides, such as 4 or 5 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide useful in the invention comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA). Often used LNA nucleosides are oxy-LNA or cET.

In a further embodiment the oligonucleotide comprises at least one modified internucleoside linkage. Suitable internucleoside modifications are described in the “Definitions” section under “Modified internucleoside linkage”. It is advantageous if at least 2 to 3 internucleoside linkages at the 5′ or 3′ end of the oligonucleotide are phosphorothioate internucleoside linkages. For single stranded antisense oligonucleotides it is advantageous if at least 75%, such as, such as all, the internucleoside linkages within the contiguous nucleotide sequence are phosphorothioate internucleoside linkages.

In a further aspect of the invention, the nucleic acid molecules, such as the antisense oligonucleotide, siRNA or shRNA, useful in the invention can be targeted directly to the liver by covalently attaching them to a conjugate moiety capable of binding to the asialoglycoprotein receptor (ASGPr), such as divalent or trivalent GalNAc cluster.

Enhanced antisense oligonucleotides useful in the invention, or conjugates thereof, are also provided and are potentially excellent FUBP1 inhibitors since they can target the FUBP1 transcript and may promote its degradation either via RNase H cleavage.

In some embodiments of the invention, the enhanced antisense oligonucleotide or conjugates thereof is capable of modulating the expression of the target by inhibiting or down-regulating it. Preferably, such modulation produces an inhibition of expression of at least 20% compared to the normal expression level of the target, more preferably at least 30%, at least 40%, at least 50%, at least 60%, at least 70%, at least 80%, or at least 90% inhibition compared to the normal expression level of the target. In some embodiments, the antisense oligonucleotide of the combination of the invention or conjugates thereof may be capable of inhibiting expression levels of FUBP1 mRNA by at least 50% or 60% in vitro using 25 μM in PXB-PHH cells. In some embodiments of the invention, the antisense oligonucleotide or conjugates thereof may be capable of inhibiting expression levels of FUBP1 protein by at least 50% in vitro using 25 μM in PXB-PHH cells, this range of target reduction is advantageous in terms of selecting antisense oligonucleotides with good correlation to the cccDNA reduction. Suitably, the examples provide assays, which may be used to measure FUBP1 RNA inhibition (e.g. Example 1 or 2). The target inhibition is triggered by the hybridization between a contiguous nucleotide sequence of the antisense oligonucleotide and the target nucleic acid. In some embodiments, the antisense oligonucleotide of the combination of the invention comprises mismatches between the antisense oligonucleotide and the target nucleic acid. Despite mismatches hybridization to the target nucleic acid may still be sufficient to show a desired inhibition of FUBP1 expression. Reduced binding affinity resulting from mismatches may advantageously be compensated by increased number of nucleotides in the oligonucleotide and/or an increased number of modified nucleosides capable of increasing the binding affinity to the target, such as 2′ sugar modified nucleosides, including LNA, present within the antisense oligonucleotide sequence.

The antisense oligonucleotide of the combination of the invention is typically 12-30, such as 12 to 22, such as 16 to 20 nucleotides in length, and comprises a contiguous nucleotide sequence of at least 12 nucleotides, such as of 13, 14, 15, 16, 17 or 18 nucleotides, which is complementary to, such as fully complementary to a region of the human FUBP1 pre-mRNA (as illustrated in SEQ ID NO: 247), selected from a region from nucleotides 9141-9156, 16184-16205, 16184-16200, 16186-16203, 16188-16205, and 16189-16205 and 30536-30553 of SEQ ID NO: 247

In some embodiments of the present invention, the antisense oligonucleotide comprises a contiguous nucleotide sequence of 12 to 22 nucleotides, such as of 15 to 20 nucleotides, with at least 90% complementarity, such as fully complementary, to the target nucleic acid of SEQ ID NO: 256.

In some embodiments, antisense oligonucleotide comprises a contiguous nucleotide sequence of 15 to 18 nucleotides, such as of 17 or 18 nucleotides, with at least 90% complementarity, such as fully complementary, to the target nucleic acid of SEQ ID NO: 257.

In some embodiments, the antisense oligonucleotide comprises a contiguous nucleotide sequence of 15 to 22 nucleotides, such as 18 to 22 nuucleotides or such as of 15 to 18 nucleotides, such as of 17 or 18 nucleotides with at least 90% complementarity, such as 91%, 92%, 93%, 94%, 95%, 96%, 97%, 98%, 99%, such as fully complementary, to the target nucleic acid selected from the following regions of SEQ ID NO: 247: 9141-9156, 16184-16205, 16184-16200, 16186-16203, 16188-16205, 16189-16205 and 30536-30553 of SEQ ID NO: 247. In some embodiments, the antisense oligonucleotide comprises a contiguous sequence of 12 to 30 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with a region of the target nucleic acid or a target sequence.

It is advantageous if the antisense oligonucleotide of the combination of the invention, or contiguous nucleotide sequence thereof is fully complementary (100% complementary) to a region of the target nucleic acid, or in some embodiments may comprise one or two mismatches between the oligonucleotide and the target nucleic acid.

In some embodiments, the antisense oligonucleotide sequence is 100% complementary to a corresponding target nucleic acid of SEQ ID NO: 247.

In some embodiments, the antisense oligonucleotide of the combination of the invention or the contiguous nucleotide sequence thereof is at least 95% complementarity, such as fully (or 100%) complementary, to the target nucleic acid of SEQ ID NO: 247 and SEQ ID NO: 250.

In some embodiments, the antisense oligonucleotide comprises a contiguous nucleotide sequence of 15 to 22 nucleotides in length with at least 90% complementary, such as 100% complementarity, to a corresponding target sequence present in SEQ ID NO: 247, wherein the target sequence is selected from nucleotides 9141-9156, 16184-16205, 16184-16200, 16186-16203, 16188-16205, 16189-16205 and 30536-30553 of SEQ ID NO: 247.

In some embodiments, the contiguous nucleotide sequence of the antisense oligonucleotide is at least 90% complementary, advantageously 100% complementary, to a target site sequence of SEQ ID NO: 256.

In some embodiments, the contiguous nucleotide sequence of the antisense oligonucleotide is at least 90% complementary, advantageously 100% complementary, to a target site sequence of SEQ ID NO: 257.

In some embodiments, the contiguous nucleotide sequence of the antisense oligonucleotide is at least 90% complementary, advantageously 100% complementary, to a target site sequence of SEQ ID NO: 261.

In some embodiments, the contiguous nucleotide sequence of the antisense oligonucleotide is at least 90% complementary, advantageously 100% complementary, to a target site sequence of SEQ ID NO: 270.

In some embodiments, the contiguous nucleotide sequence comprises a sequence of nucleobases selected from the group consisting of SEQ ID NO: 325, 326, 327, 328, 329 and 330, or at least 14 contiguous nucleotides thereof, such as 17 or 18 contiguous nucleotides thereof.

In some embodiments, the antisense oligonucleotide of the combination of the invention or contiguous nucleotide sequence thereof, comprises or consists of 10 to 30 nucleotides in length, such as from 12 to 25, such as 11 to 22, such as from 12 to 20, such as from 14 to 18 or 16 to 18 contiguous nucleotides in length.

In some embodiments, the antisense oligonucleotide or contiguous nucleotide sequence thereof comprises or consists of 22 or less nucleotides, such as 20 or less, or 18 or less nucleotides. For example, antisense oligonucleotide or contiguous nucleotide sequence thereof may comprise 14, 15, 16 or 17 nucleotides. It is to be understood that any range given herein includes the range endpoints. Accordingly, if an oligonucleotide is said to include from 10 to 30 nucleotides, both 10 and 30 nucleotides are included.

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 325.

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 326

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 327.

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 328.

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 329.

The invention provides antisense oligonucleotides, such as antisense oligonucleotides 12-24 nucleotides in length, such as 12-18 nucleotides in length, wherein the antisense oligonucleotide comprises a contiguous nucleotide sequence comprising at least 12, such as at least 13, such as at least 14, such as at least 15 or at least 16 contiguous nucleotides present in SEQ ID NO: 330.

In some embodiments, the contiguous nucleotide sequence comprises or consists of 12, 13, 14, 15, 16, 17, 18, 19, 20, 21 or 22 contiguous nucleotides in length, such as 16, 17 or 18 contiguous nucleotides.

In some embodiments, the antisense oligonucleotide or contiguous nucleotide sequence thereof comprises or consists of a sequence selected from SEQ ID NO: 325, 326, 327, 328, 329 and 330.

In advantageous embodiments, the antisense oligonucleotide comprises one or more sugar modified nucleosides, such as one or more 2′ sugar modified nucleosides, such as one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In some embodiments, the contiguous nucleotide sequence comprises LNA nucleosides.

In some embodiments, the contiguous nucleotide sequence comprises LNA nucleosides and DNA nucleosides.

In some embodiments, the contiguous nucleotide sequence comprises 2′-O-methoxyethyl (2′MOE) nucleosides.

In some embodiments, the contiguous nucleotide sequence comprises 2′-O-methoxyethyl (2′MOE) nucleosides and DNA nucleosides.

Advantageously, the 3′ most nucleoside of the antisense oligonucleotide, or contiguous nucleotide sequence thereof is a 2′ sugar modified nucleoside.

Advantageously, the antisense oligonucleotide comprises at least one modified internucleoside linkage, such as phosphorothioate or phosphorodithioate.

In some embodiments, the at least one internucleoside linkage in the contiguous nucleotide sequence is a phosphorothioate internucleoside linkages.

In some embodiments, at least one internucleoside linkage in the contiguous nucleotide sequence is a phosphorodithioate internucleoside linkages.

In some embodiments, at least one internucleoside linkage in the contiguous nucleotide sequence is a phosphodiester internucleoside linkages.

In some embodiments, all the internucleoside linkages within the contiguous nucleotide sequence are phosphorothioate internucleoside linkages.

In some embodiments, at least 75% the internucleoside linkages within the antisense oligonucleotide, or contiguous nucleotide sequence thereof, are phosphorothioate internucleoside linkages.

In some embodiments, all the internucleoside linkages within the antisense oligonucleotide, or contiguous nucleotide sequence thereof, are phosphorothioate internucleoside linkages.

In an advantageous embodiment of the invention the antisense oligonucleotide of the combination of the invention is capable of recruiting RNase H, such as RNase H1. In some embodiments of the invention, the antisense oligonucleotide of the combination of the invention, or the contiguous nucleotide sequence thereof is a gapmer.

In some embodiments, the antisense oligonucleotide, or contiguous nucleotide sequence thereof, consists or comprises a gapmer of formula 5′-F-G-F′-3′.

In some embodiments, region G consists of 6-16 DNA nucleosides, such as 7 to 12 DNA nucleosides. In some embodiments, region F comprises 4 to 6 nucleosides and/or region F′ comprises 2 to 6 nucleosides.

In some embodiments, region F and F′ each comprise at least one LNA nucleoside.

In some embodiments of the oligonucleotide of the present invention, all LNA nucleosides are beta-D-oxy LNA nucleosides.

In some embodiments, the oligonucleotide of the present invention is a LNA gapmer with uniform flanks.

In some embodiments of the invention, the LNA gapmer is an alternating flank LNA gapmer. In some embodiments, the alternating flank LNA gapmer comprises at least one alternating flank (such as flank F). In some embodiments, the alternating flank LNA gapmer comprises one alternating flank (such as flank F) and one uniform flank (such as flank F′). In some embodiments, the alternating flank LNA gapmer comprises two alternating flanks. For example, the LNA gapmer may have a design selected from the following designs: 1-12-3, 3-2-1-9-2, 3-1-1-10-2, 2-1-2-10-3, 2-1-1-11-3, 2-1-1-10-1-1-2, 2-1-1-10-4, 1-3-1-7-1-1-3, 3-2-1-9-3, and 1-1-3-9-1-1-2. Table 12B lists preferred designs for each motif sequence.

The invention provides the following oligonucleotide compounds (Table 12B):

TABLE 12B
list of oligonucleotide motif sequences of the combination of the invention
(indicated by SEQ ID NO), designs of these, as well as specific oligonucleotide
compounds of the combination of the invention (indicated by CMP ID NO)
designed based on the motif sequence.

| | | position | | | | |
|---|---|---|---|---|---|---|
| SEQ | | on SEQ ID | | | CMP | |
| ID | | NO: 247 | | | ID | Oligonucleotide |
| NO | Motif sequence | Start | end | Design | NO | Compound |
| 325 | CTTATGCTTTTTAT | 16189 | 16205 | 3-2-1- | 325_1 | CTTatGctttttatgGT |
| | GGT | | | 9-2 | | |
| 325 | CTTATGCTTTTTAT | 16189 | 16205 | 3-1-1- | 325_2 | CTTaTgctttttatgGT |
| | GGT | | | 10-2 | | |
| 326 | CTTATGCTTTTTAT | 16188 | 16205 | 2-1-2- | 326_1 | CTtATgctttttatgGTT |
| | GGTT | | | 10-3 | | |
| 326 | CTTATGCTTTTTAT | 16188 | 16205 | 2-1-1- | 326_2 | CTtAtgctttttatgGTT |
| | GGTT | | | 11-3 | | |
| 326 | CTTATGCTTTTTAT | 16188 | 16205 | 2-1-1- | 326_3 | CTtAtgctttttatGgTT |
| | GGTT | | | 10-1- | | |
| | | | | 1-2 | | |
| 326 | CTTATGCTTTTTAT | 16188 | 16205 | 2-1-1- | 326_4 | CTtAtgctttttatGGTT |
| | GGTT | | | 10-4 | | |
| 327 | GCTTTTTATGGTTT | 16184 | 16200 | 1-3-1- | 327_1 | GcttTttatggtTtCAC |
| | CAC | | | 7-1-1- | | |
| | | | | 3 | | |
| 328 | TATGCTTTTTATGG | 16186 | 16203 | 3-2-1- | 328_1 | TATgcTttttatggtTTC |
| | TTTC | | | 9-3 | | |
| 329 | ACCAATTTTCATTT | 30536 | 30553 | 1-1-3- | 329_1 | AcCAAttttcatttCtAC |
| | CTAC | | | 9-1-1- | | |
| | | | | 2 | | |
| 330 | CCCCATAACCATA | 9141 | 9156 | 1-12-3 | 330_1 | CcccataaccataGTC |
| | GTC | | | | | |

The heading “Oligonucleotide compound” in the table 12A and 12B represents specific designs of a motif sequence. Capital letters are beta-D-oxy LNA nucleosides, lowercase letters are DNA nucleosides, all LNA C are 5-methyl cytosine, all internucleoside linkages are phosphorothioate internucleoside linkages. The heading “Designs” refers to the gapmer design, F-G-F′. In gapmers with alternating flank designs the flanks of the oligonucleotide are annotated as a series of integers, representing a number of beta-D-oxy LNA nucleosides (L) followed by a number of DNA nucleosides (D). For example, a flank with a 2-2-1 motif represents LLDDL. Both flanks have a beta-D-oxy LNA nucleoside at the 5′ and 3′ terminal. The gap region (G), which is constituted of a number of DNA nucleosides is located between the flanks.

For some embodiments of the invention, the oligonucleotide is selected from the group of oligonucleotide compounds with CMP-ID-NO: 325_1, 325_2, 326_1, 326_2, 326_3, 326_4, 327_1, 328_1, 329_1 and 330_1 (see Table 12B).

In a special embodiment, the compound of the combination of the invention is the compound with CMP ID NO: 325_1 (see Table 12B).

In a special embodiment, the compound of the combination of the invention is the compound with CMP ID NO: 325_2 (see Table 12B).

In a special embodiment, the compound of the combination of the invention is the compound with CMP ID NO: 326_1 (see Table 12B).

In a special embodiment, the compound of the combination of the invention is the compound with CMP ID NO: 326_2 (see Table 12B).

In a special embodiment, the compound of the combination of the invention is the compound with CMP ID NO: 326_3 (see Table 12B).

In a special embodiment, the compound of the combination of the invention is the compound with CMP ID NO: 326_4 (see Table 12B).

In a special embodiment, the compound of the combination of the invention is the compound with CMP ID NO: 327_1 (see Table 12B).

In a special embodiment, the compound of the combination of the invention is the compound with CMP ID NO: 328_1 (see Table 12B).

In a special embodiment, the compound of the combination of the invention is the compound with CMP ID NO: 329_1 (see Table 12B).

In a special embodiment, the compound of the combination of the invention is the compound with CMP ID NO: 330_1 (see Table 12B).

The antisense oligonucleotide may be selected from the group listed in Table 13, or a pharmaceutically acceptable salt thereof.

TABLE 13
Compound Table (Exemplary antisense oligonucleotides of the present invention) -
HELM Annotation Format

| | | | Comprised |
|---|---|---|---|
| | | | by |
| SEQ | Compound | | conjugate |
| ID | ID | HELM Annotation | shown in |
| Number | Number # | Written 5′ - 3′. | FIG. |
| 325 | 325_1 | {[LR]([5meC])[sP].[LR](T)[sP].[LR](T)[sP].[dR](A)[sP].[dR](T) | 1 |
| | | [sP].[LR](G)[sP].[dR](C)[sP].[dR](T)[sP].[dR](T)[sP].[dR](T) | |
| | | [sP].[dR](T)[sP].[dR](T)[sP].[dR](A)[sP].[dR](T)[sP].[dR](G) | |
| | | [sP].[LR](G)[sP].[LR](T)} | |
| 325 | 325_2 | {[LR]([5meC])[sP].[LR](T)[sP].[LR](T)[sP].[dR](A)[sP].[LR](T) | 2 |
| | | [sP].[dR](G)[sP].[dR](C)[sP].[dR](T)[sP].[dR](T)[sP].[dR](T) | |
| | | [sP].[dR](T)[sP].[dR](T)[sP].[dR](A)[sP].[dR](T)[sP].[dR](G) | |
| | | [sP].[LR](G)[sP].[LR](T)} | |
| 326 | 326_1 | {[LR]([5meC])[sP].[LR](T)[sP].[dR](T)[sP].[LR](A)[sP].[LR](T) | 3 |
| | | [sP].[dR](G)[sP].[dR](C)[sP].[dR](T)[sP].[dR](T)[sP].[dR](T) | |
| | | [sP].[dR](T)[sP].[dR](T)[sP].[dR](A)[sP].[dR](T)[sP].[dR](G) | |
| | | [sP].[LR](G)[sP].[LR](T)[sP].[LR](T)} | |
| 326 | 326_2 | {[LR]([5meC])[sP].[LR](T)[sP].[dR](T)[sP].[LR](A)[sP].[dR](T) | 4 |
| | | [sP].[dR](G)[sP].[dR](C)[sP].[dR](T)[sP].[dR](T)[sP].[dR](T) | |
| | | [sP].[dR](T)[sP].[dR](T)[sP].[dR](A)[sP].[dR](T)[sP].[dR](G) | |
| | | [sP].[LR](G)[sP].[LR](T)[sP].[LR](T)} | |
| 326 | 326_3 | {[LR]([5meC])[P].[LR](T)[P].[dR](T)[P].[LR](A)[sP].[dR](T) | 5 |
| | | [sP].[dR](G)[sP].[dR](C)[sP].[dR](T)[sP].[dR](T)[sP].[dR](T) | |
| | | [sP].[dR](T)[sP].[dR](T)[sP].[dR](A)[sP].[dR](T)[sP].[LR](G) | |
| | | [sP].[dR](G)[sP].[LR](T)[sP].[LR](T)} | |
| 326 | 326_4 | {[LR]([5meC])[sP].[LR](T)[P].[dR](T)[sP].[LR](A)[sP].[dR](T) | 6 |
| | | [sP].[dR](G)[sP].[dR](C)[P].[dR](T)[sP].[dR](T)[P].[dR](T) | |
| | | [sP].[dR](T)[sP].[dR](T)[sP].[dR](A)[sP].[dR](T)[sP].[LR](G) | |
| | | [sP].[LR](G)[sP].[LR](T)[sP].[LR](T)} | |
| 327 | 327_1 | {[LR](G)[sP].[dR](C)[sP].[dR](T)[sP].[dR](T)[sP].[LR](T)[P]. | 7 |
| | | [dR](T)[sP].[dR](T)[sP].[dR](A)[sP].[dR](T)[sP].[dR](G)[sP]. | |
| | | [dR](G)[sP].[dR](T)[sP].[LR](T)[P].[dR](T)[sP].[LR]([5meC]) | |
| | | [sP].[LR](A)[sP].[LR]([5meC])} | |
| 328 | 328_1 | {[LR](T)[sP].[LR](A)[sP].[LR](T)[sP].[dR](G)[sP].[dR](C)[P]. | 8 |
| | | [LR](T)[sP].[dR](T)[sP].[dR](T)[sP].[dR](T)[sP].[dR](T)[sP]. | |
| | | [dR](A)[sP].[dR](T)[sP].[dR](G)[P].[dR](G)[sP].[dR](T)[sP]. | |
| | | [LR](T)[sP].[LR](T)[sP].[LR]([5meC])} | |
| 329 | 329_1 | {[LR](A)[P].[dR](C)[sP].[LR]([5meC])[sP].[LR](A)[sP].[LR] | 8.1 |
| | | (A)[sP].[dR](T)[sP].[dR](T)[sP].[dR](T)[sP].[dR](T)[sP].[dR] | |
| | | (C)[sP].[dR](A)[sP].[dR](T)[sP].[dR](T)[sP].[dR](T)[sP].[LR] | |
| | | ([5meC])[sP].[dR](T)[sP].[LR](A)[sP].[LR]([5meC])} | |
| 330 | 330_1 | {[LR]([5meC])[sP].[dR](C)[sP].[dR](C)[P].[dR](C)[sP].[dR] | |
| | | (A)[sP].[dR](T)[sP].[dR](A)[sP].[dR](A)[sP].[dR](C)[sP].[dR] | |
| | | (C)[sP].[dR](A)[sP].[dR](T)[sP].[dR](A)[P].[LR](G)[sP].[LR] | |
| | | (T)[sP].[LR]([5meC])} | |

### Helm Annotation Key:

[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC] is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR]([C] is a DNA cytosine nucleoside,
[sP]. is a phosphorothioate internucleoside linkage,
P. is a phosphodiester internucleoside linkage.

The invention thus provides for an antisense oligonucleotide selected from the group consisting of compound ID Nos #325_1, 325_2, 326_1, 326_2, 326_3, 326_4, 327_1, 328_1, 329_1 and 330_1.

In all instances, the F-G-F′ design may further include region D′ and/or D″ as described in the “Definitions” section under “Region D′ or D” in an oligonucleotide”. In some embodiments, the oligonucleotide of the combination of the invention has 1, 2 or 3 phosphodiester linked nucleoside units, such as DNA units, at the 5′ or 3′ end, such as at the 5′ end, of the gapmer region. In some embodiments, the oligonucleotide of the combination of the invention consists of two 5′ phosphodiester linked DNA nucleosides followed by a F-G-F′ gapmer region as defined above. Oligonucleotides that contain phosphodiester linked DNA units at the 5′ or 3′ end are suitable for conjugation and may further comprise a conjugate moiety as described herein. For delivery to the liver ASGPR targeting moieties are particular advantageous as conjugate moieties, see the Conjugate section for further details

### Combination

In one aspect, a third category of compound in the combination of the invention is an oligonucleotide targeting RTEL1 linked by a linker to an oligopnucleotide targeting FUBP1.

In one embodiment, the linker consists of a DNA dinucleotide with a sequence selected from the group consisting of AA, AT, AC, AG, TA, TT, TC, TG, CA, CT, CC, CG, GA, GT, GC, or GG, where there is a phosphodiester linkage between the two DNA nucleosides. Preferably, the linker is a CA DNA dinucleotide.

In one embodiment, the linkage at the 5′ end of the dinucleotide-linking the dinucleotide to one of the oligonucleotides targeting RTEL1 or FUBP1—is a phosphodiester linkage or a phosphorothioate linkage; and the linkage at the 3′ end of the dinucleotide-linking the dinucleotide to the another oligonucleotides targeting RTEL1 or FUBP1—is a phosphodiester linkage or a phosphorothioate linkage.

In one embodiment, an oligonucleotide targeting RTEL1 is linked on its 3′end to the 5′end of an oligonucleotide targeting FUBP1 via a CA DNA dinucleotide, wherein the linkage between the 3′end of the oligonucleotide targeting RTEL1 and the 5′end of the dinucleotide is a phosphodiester linkage; and wherein the linkage between the 3′ end of the dinucleotide and the 5′end of the oligonucleotide targeting FUBP1 is a phosphodiester linkage.

In one embodiment, an oligonucleotide targeting FUBP1 is linked on its 3′end to the 5′end of an oligonucleotide targeting RTEL1 via a CA DNA dinucleotide, wherein the linkage between the 3′end of the oligonucleotide targeting FUBP1 and the 5′end of the dinucleotide is a phosphodiester linkage; and wherein the linkage between the 3′ end of the dinucleotide and the 5′end of the oligonucleotide targeting RTEL1 is a phosphodiester linkage.

In one embodiment, an oligonucleotide targeting RTEL1 is linked on its 3′end to the 5′end of an oligonucleotide targeting FUBP1 via a CA DNA dinucleotide, wherein the linkage between the 3′end of the oligonucleotide targeting RTEL1 and the 5′end of the dinucleotide is a phosphorothioate linkage; and wherein the linkage between the 3′ end of the dinucleotide and the 5′end of the oligonucleotide targeting FUBP1 is a phosphodiester linkage.

In one embodiment, an oligonucleotide targeting FUBP1 is linked on its 3′end to the 5′end of an oligonucleotide targeting RTEL1 via a CA DNA dinucleotide, wherein the linkage between the 3′end of the oligonucleotide targeting FUBP1 and the 5′end of the dinucleotide is a phosphorothioate linkage; and wherein the linkage between the 3′ end of the dinucleotide and the 5′end of the oligonucleotide targeting RTEL1 is a phosphodiester linkage.

In one embodiment, the 5′ end most oligonucleotide of the combination consisting of an oligonucleotide targeting RTEL1 linked by a linker to an oligopnucleotide targeting FUBP1, is further linked by a linker to a conjugate moiety.

In one embodiment, the conjugate moiety is linked to the 5′ end most oligonucleotide by a linker which consists of a DNA dinucleotide with a sequence selected from the group consisting of AA, AT, AC, AG, TA, TT, TC, TG, CA, CT, CC, CG, GA, GT, GC, or GG, where there is a phosphodiester linkage between the two DNA nucleosides. Preferably, the linker is a CA DNA dinucleotide.

In one embodiment, the linkage at the 5′ end of the dinucleotide-linking the dinucleotide to the conjugate moiety—is a phosphodiester linkage or a phosphorothioate linkage; and the linkage at the 3′ end of the dinucleotide-linking the dinucleotide to the 5′ end of the 5′ most oligonucleotide—is a phosphodiester linkage or a phosphorothioate linkage.

In one embodiment, the 5′ most oligonucleotide is an oligonucleotide targeting RTEL1 which is linked on its 5′ end to a conjugate moiety via a CA DNA dinucleotide, wherein the linkage between the 5′ end of the oligonucleotide targeting RTEL1 and the 3′ end of the dinucleotide is a phosphodiester linkage; and wherein the linkage between the 5′ end of the dinucleotide and the conjugate moiety is a phosphodiester linkage.

In one embodiment, the 5′ most oligonucleotide is an oligonucleotide targeting FUBP1 which is linked on its 5′ end to a conjugate moiety via a CA DNA dinucleotide, wherein the linkage between the 5′ end of the oligonucleotide targeting FUBP1 and the 3′ end of the dinucleotide is a phosphodiester linkage; and wherein the linkage between the 5′ end of the dinucleotide and the conjugate moiety is a phosphodiester linkage.

In one embodiment, the oligonucleotide targeting RTEL1 is CMP ID NO 245_1 (SEQ ID NO: 245) or CMP ID NO 246_2 (SEQ ID NO: 246).

In one embodiment, the oligonucleotide targeting FUBP1 is CMP ID NO: 326_3 (SEQ ID NO: 326) or CMP ID NO: 330_1 (SEQ ID NO: 330).

TABLE 12C
list of the sequences of combinations of oligonucleotides (indicated by SEQ ID NO),
designs of these, as well as specific combination compounds (indicated by CMP ID NO).

| SEQ | | | | CMP |
|---|---|---|---|---|
| ID | | | | ID |
| NO | Motif sequence | Design | Combination Compound | NO |
| 348 | ccccataaccatagtccactttattata | 1-12-3-2-2- | CcccataaccataGTCcaCTttattataac | 348_1 |
| | acttgaatctc | 15-4 | ttgaaTCTC | |
| 349 | ctttattataacttgaatctccaccccat | 2-15-4-2-1- | CTttattataacttgaaTCTCcaCcccata | 349_1 |
| | aaccatagtc | 12-3 | accataGTC | |
| 350 | cttatgctttttatggttcattacatact | 2-1-1-10-1- | CTtAtgctttttatGgTTcaTTacatactct | 350_1 |
| | ctggtcaaa | 1-2-2-12-4 | ggtCAAA | |
| 351 | ttacatactctggtcaaacacttatgcttt | 2-12-4-2-2- | TTacatactctggtCAAAcaCTtAtgctttt | 351_1 |
| | ttatggtt | 1-1-10-1-1- | tatGgTT | |
| | | 2 | | |

TABLE 12D
Combination of Compound Table (Exemplary combination of the present invention) -
HELM Annotation Format

| SEQ | Oligo | |
|---|---|---|
| ID | Compound | |
| Number | ID | HELM Annotation (Written 5′ - 3′.) |
| 348 | 348_1 | RNA1{[LR]([5meC])[sP].[dR](C)[sP].[dR](C)[sP].[dR](C)[sP].[dR](A)[sP].[dR](T)[s |
| | | P].[dR](A)[sP].[dR](A)[sP].[dR](C)[sP].[dR](C)[sP].[dR](A)[sP].[dR](T)[sP].[dR](A) |
| | | [sP].[LR](G)[sP].[LR](T)[sP].[LR]([5meC])P.[dR](C)P.[dR](A)P.[LR]([5meC])[sP]. |
| | | [LR](T)[P].[dR](T)[sP].[dR](T)[sP].[dR](A)[sP].[dR](T)[sP].[dR](T)[sP].[dR](A)[sP] |
| | | .[dR](T)[sP].[dR](A)[sP].[dR](A)[sP].[dR](C)[sP].[dR](T)[sP].[dR](T)[sP].[dR](G)[s |
| | | P].[dR](A)[sP].[dR](A)[sP].[LR](T)[sP].[LR]([5meC])[P].[LR](T)[sP].[LR]([5meC])} |
| | | $$$$V2.0 |
| 348 | 348_2 | RNA1{[LR]([5meC])[sP].[dR](C)[sP].[dR](C)[sP].[dR](C)[sP].[dR](A)[sP].[dR](T)[s |
| | | P].[dR](A)[sP].[dR](A)[sP].[dR](C)[sP].[dR](C)[sP].[dR](A)[sP].[dR](T)[sP].[dR](A) |
| | | [sP].[LR](G)[sP].[LR](T)[sP].[LR]([5meC])[sP].[dR](C)P.[dR](A)P.[LR]([5meC])[s |
| | | P].[LR](T)[sP].[dR](T)[sP].[dR](T)[sP].[dR](A)[sP].[dR](T)[sP].[dR](T)[sP].[dR](A) |
| | | [sP].[dR](T)[sP].[dR](A)[sP].[dR](A)[sP].[dR](C)[sP].[dR](T)[sP].[dR](T)[sP].[dR] |
| | | (G)[sP].[dR](A)[sP].[dR](A)[sP].[LR](T)[sP].[LR]([5meC])[sP].[LR](T)[sP].[LR] |
| | | ([5meC])}$$$$V2.0 |
| 349 | 349_1 | RNA1{[LR]([5meC])[P].[LR](T)[sP].[dR](T)[sP].[dR](T)[sP].[dR](A)[sP].[dR](T)[s |
| | | P].[dR](T)[sP].[dR](A)[sP].[dR](T)[sP].[dR](A)[sP].[dR](A)[P].[dR](C)[sP].[dR](T) |
| | | [sP].[dR](T)[sP].[dR](G)[sP].[dR](A)[P].[dR](A)[sP].[LR](T)[P].[LR]([5meC])[sP] |
| | | [LR](T)[sP].[LR]([5meC])P.[dR](C)P.[dR](A)P.[LR]([5meC])[sP].[dR](C)[sP].[dR] |
| | | (C)[sP].[dR](C)[sP].[dR](A)[sP].[dR](T)[sP].[dR](A)[sP].[dR](A)[sP].[dR](C)[sP].[d |
| | | R](C)[sP].[dR](A)[sP].[dR](T)[sP].[dR](A)[sP].[LR](G)[sP].[LR](T)[sP].[LR] |
| | | ([5meC])}$$$$V2.0 |
| 349 | 349_2 | RNA1{[LR]([5meC])[P].[LR](T)[sP].[dR](T)[sP].[dR](T)[sP].[dR](A)[sP].[dR](T)[s |
| | | P].[dR](T)[sP].[dR](A)[sP].[dR](T)[sP].[dR](A)[sP].[dR](A)[sP].[dR](C)[sP].[dR](T) |
| | | [sP].[dR](T)[sP].[dR](G)[sP].[dR](A)[sP].[dR](A)[sP].[LR](T)[sP].[LR]([5meC])[sP] |
| | | .[LR](T)[sP].[LR]([5meC])[sP].[dR](C)P.[dR](A)P.[LR]([5meC])[sP].[dR](C)[sP].[d |
| | | R](C)[sP].[dR](C)[sP].[dR](A)[sP].[dR](T)[sP].[dR](A)[sP].[dR](A)[sP].[dR](C)[sP]. |
| | | [dR](C)[sP].[dR](A)[sP].[dR](T)[sP].[dR](A)[sP].[LR](G)[P].[LR](T)[sP].[LR] |
| | | ([5meC])}$$$$V2.0 |
| 350 | 350_1 | RNA1{[LR]([5meC])[sP].[LR](T)[sP].[dR](T)[sP].[LR](A)[sP].[dR](T)[sP].[dR](G)[s |
| | | P].[dR](C)[sP].[dR](T)[sP].[dR](T)[P].[dR](T)[sP].[dR](T)[sP].[dR](T)[sP].[dR](A) |
| | | [sP].[dR](T)[sP].[LR](G)[sP].[dR](G)[sP].[LR](T)[sP].[LR](T)P.[dR](C)P.[dR](A)P. |
| | | [LR](T)[sP].[LR](T)[sP].[dR](A)[sP].[dR](C)[sP].[dR](A)[sP].[dR](T)[sP].[dR](A)[sP]. |
| | | [dR](C)[sP].[dR](T)[sP].[dR](C)[sP].[dR](T)[sP].[dR](G)[sP].[dR](G)[sP].[dR](T) |
| | | [sP].[LR]([5meC])[sP].[LR](A)[sP].[LR](A)[sP].[LR](A)}$$$$V2.0 |
| 351 | 351_1 | RNA1{[LR](T)[sP].[LR](T)[sP].[dR](A)[P].[dR](C)[P].[dR](A)[sP].[dR](T)[sP].[dR] |
| | | (A)[sP].[dR](C)[sP].[dR](T)[sP].[dR](C)[sP].[dR](T)[sP].[dR](G)[sP].[dR](G)[sP]. |
| | | [dR](T)[sP].[LR]([5meC])[sP].[LR](A)[sP].[LR](A)[sP].[LR](A)P.[dR](C)P.[dR](A)P. |
| | | [LR]([5meC])[sP].[LR](T)[sP].[dR](T)[sP].[LR](A)[sP].[dR](T)[sP].[dR](G)[sP].[dR] |
| | | (C)[sP].[dR](T)[sP].[dR](T)[sP].[dR](T)[sP].[dR](T)[sP].[dR](T)[sP].[dR](A)[sP].[d |
| | | R](T)[sP].[LR](G)[sP].[dR](G)[sP].[LR](T)[sP].[LR](T)}$$$$V2.0 |

### Conjugates

Since HBV infection primarily affects the hepatocytes in the liver it is advantageous to conjugate the RTEL1 and/or FUBP1 inhibitor(s) useful in the invention to a conjugate moiety that will increase the delivery of the inhibitor to the liver compared to the unconjugated inhibitor. In one embodiment, liver targeting moieties are selected from moieties comprising cholesterol or other lipids or conjugate moieties capable of binding to the asialoglycoprotein receptor (ASGPR).

In some embodiments of the invention, a conjugate comprises an antisense oligonucleotide covalently attached to a conjugate moiety.

The asialoglycoprotein receptor (ASGPR) conjugate moiety comprises one or more carbohydrate moieties capable of binding to the asialoglycoprotein receptor (ASPGR targeting moieties) with affinity equal to or greater than that of galactose. The affinities of numerous galactose derivatives for the asialoglycoprotein receptor have been studied (see for example: Jobst, S. T. and Drickamer, K. JB. C. 1996, 271, 6686) or are readily determined using methods typical in the art.

In one embodiment, the conjugate moiety comprises at least one asialoglycoprotein receptor targeting moiety selected from group consisting of galactose, galactosamine, N-formyl-galactosamine, N-acetylgalactosamine, N-propionyl-galactosamine, N-n-butanoyl-galactosamine and N-isobutanoylgalactosamine. Advantageously the asialoglycoprotein receptor targeting moiety is N-acetylgalactosamine (GalNAc).

To generate the ASGPR conjugate moiety the ASPGR targeting moieties (preferably GalNAc) can be attached to a conjugate scaffold. Generally, the ASPGR targeting moieties can be at the same end of the scaffold. In one embodiment, the conjugate moiety consists of two to four terminal GalNAc moieties linked to a spacer, which links each GalNAc moiety to a brancher molecule that can be conjugated to the antisense oligonucleotide.

In a further embodiment, the conjugate moiety is mono-valent, di-valent, tri-valent or tetra-valent with respect to asialoglycoprotein receptor targeting moieties. Advantageously the asialoglycoprotein receptor targeting moiety comprises N-acetylgalactosamine (GalNAc) moieties.

GalNAc conjugate moieties can include, for example, those described in WO 2014/179620 and WO 2016/055601 and PCT/EP2017/059080 (hereby incorporated by reference), as well as small peptides with GalNAc moieties attached such as Tyr-Glu-Glu-(aminohexyl GalNAc) 3 (YEE (ahGalNAc) 3; a glycotripeptide that binds to asialoglycoprotein receptor on hepatocytes, see, e.g., Duff, et al., Methods Enzymol, 2000, 313, 297); lysine-based galactose clusters (e.g., L3G4; Biessen, et al., Cardovasc. Med., 1999, 214); and cholane-based galactose clusters (e.g., carbohydrate recognition motif for asialoglycoprotein receptor).

The ASGPR conjugate moiety, in particular a trivalent GalNAc conjugate moiety, may be attached to the 3′- or 5′-end of the oligonucleotide using methods known in the art. In one embodiment, the ASGPR conjugate moiety is linked to the 5′-end of the oligonucleotide.

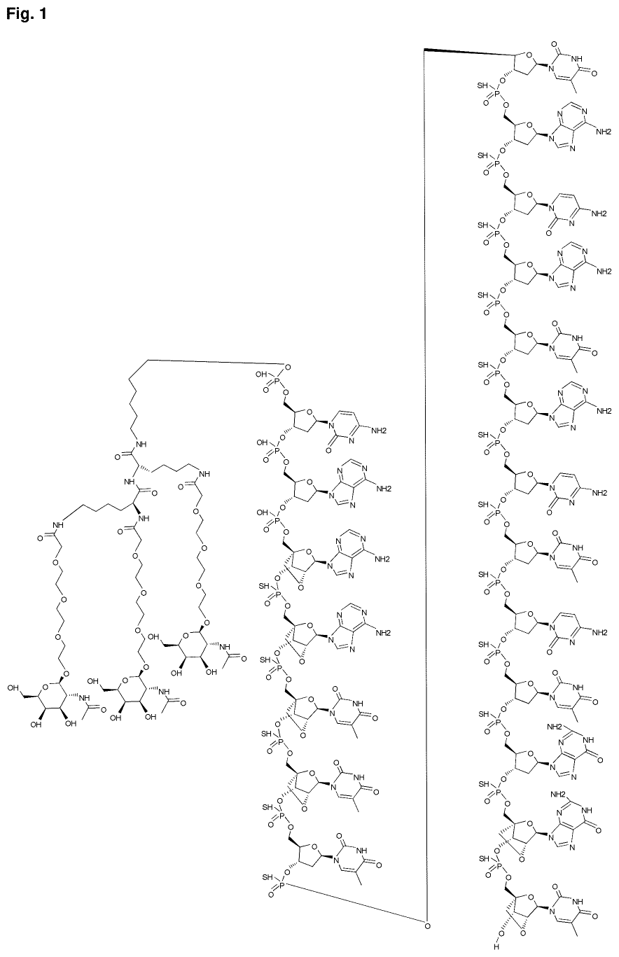

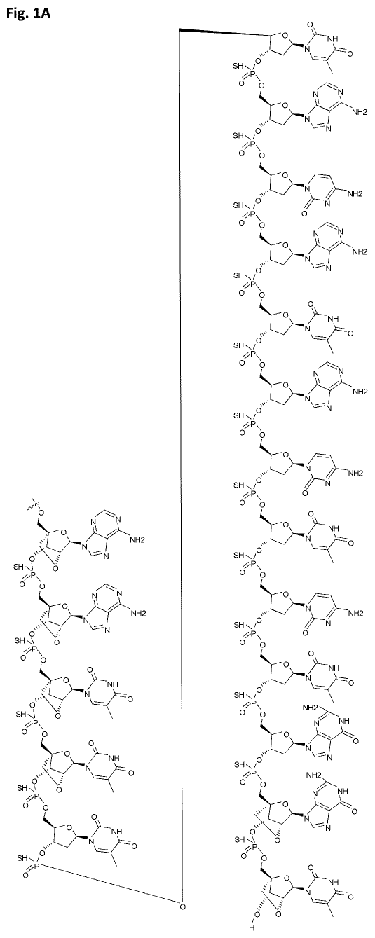

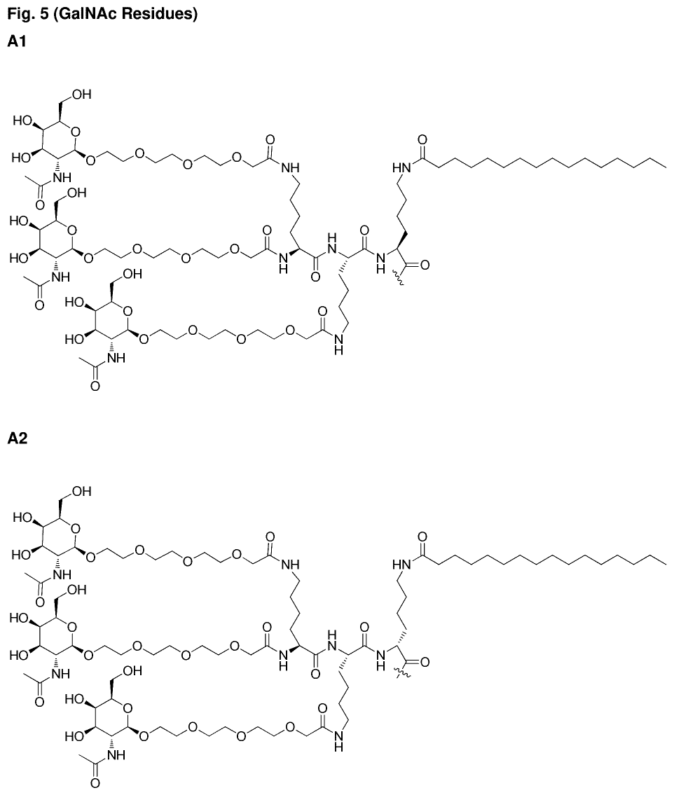

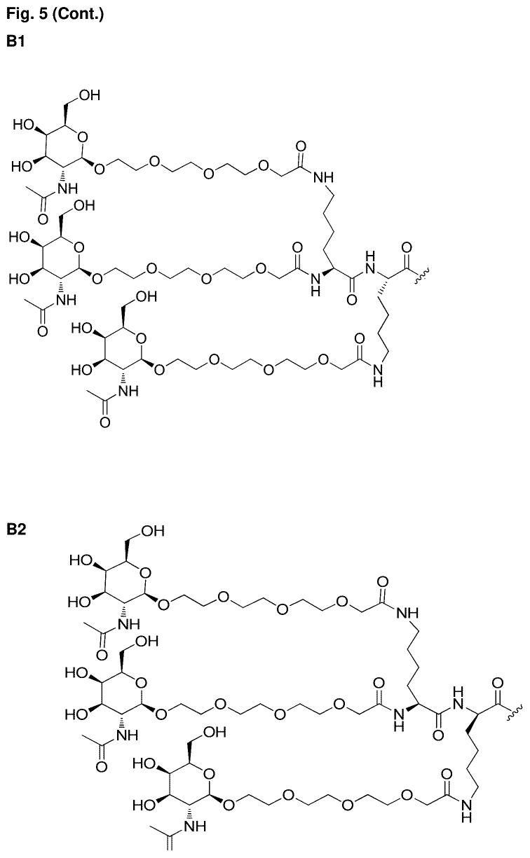

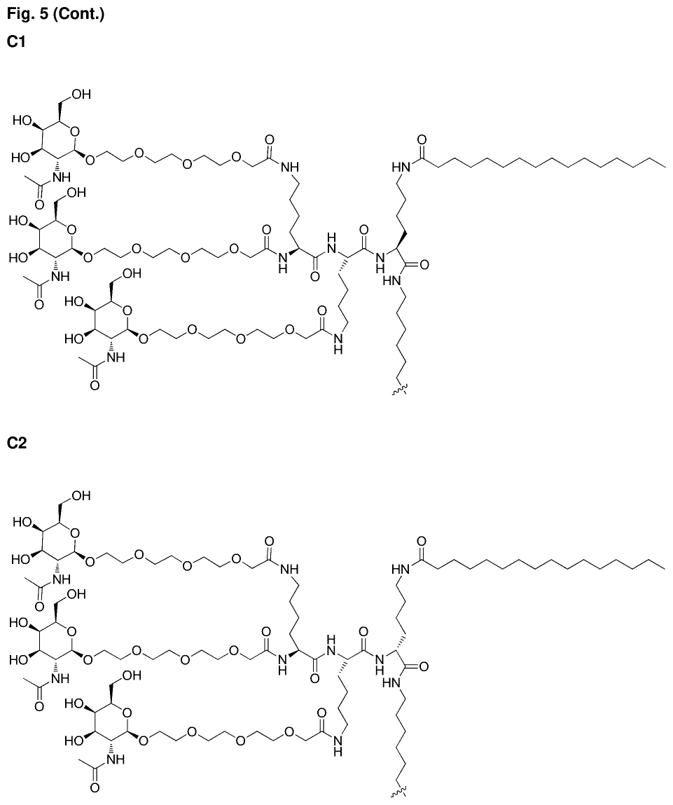

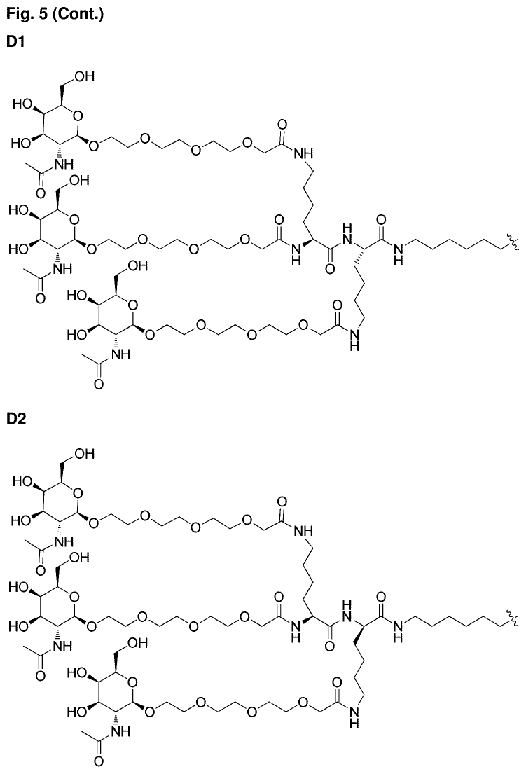

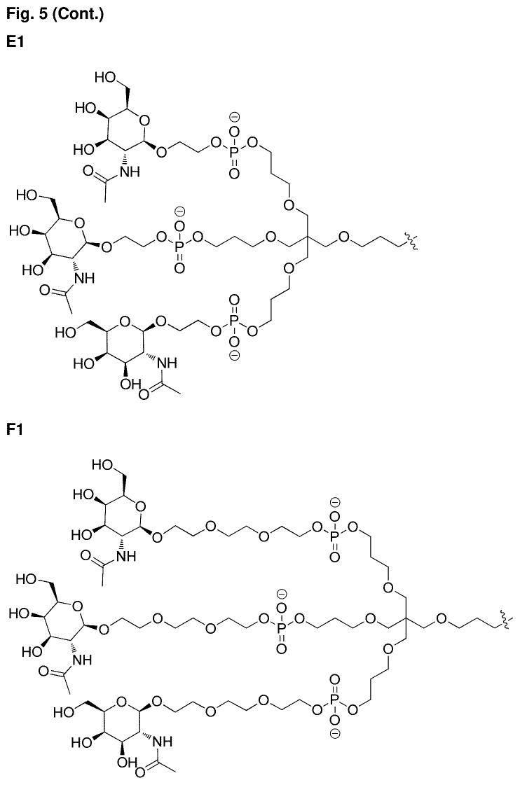

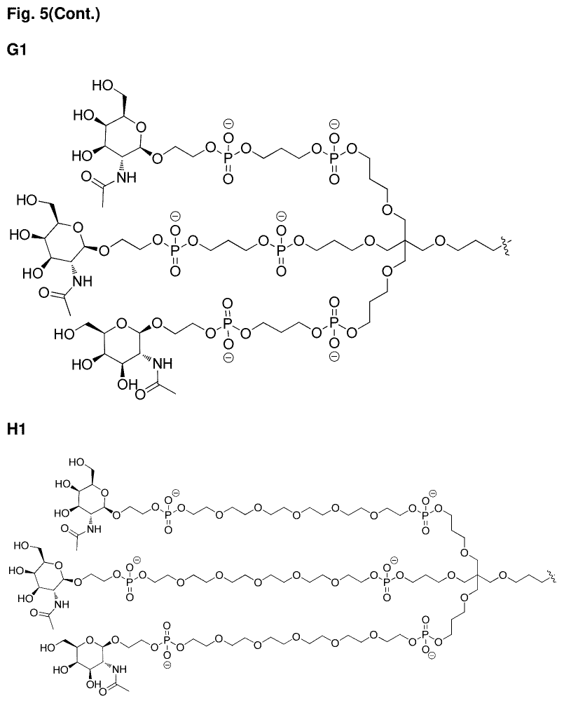

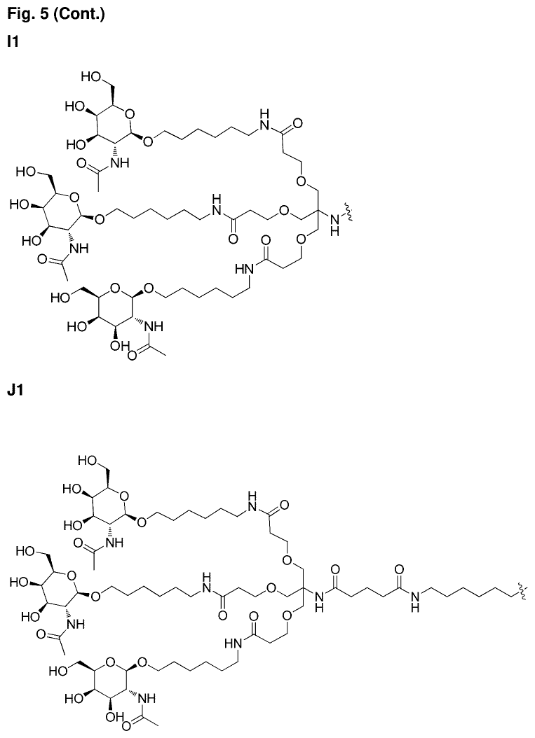

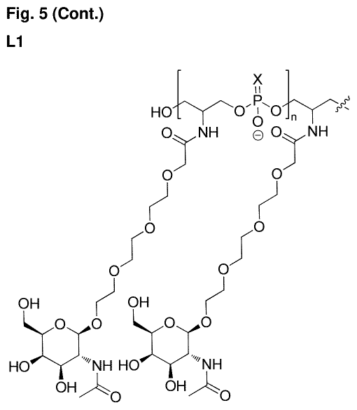

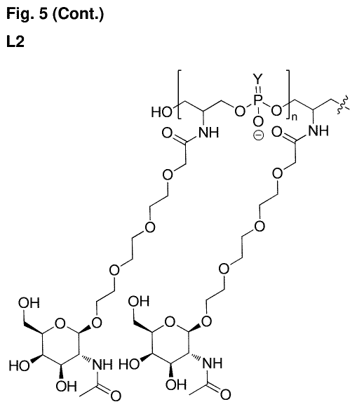

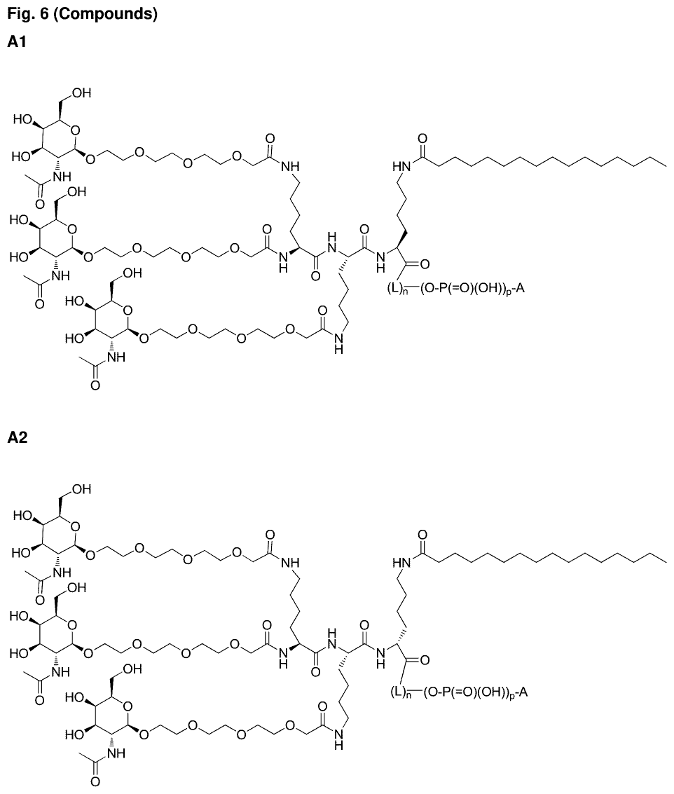

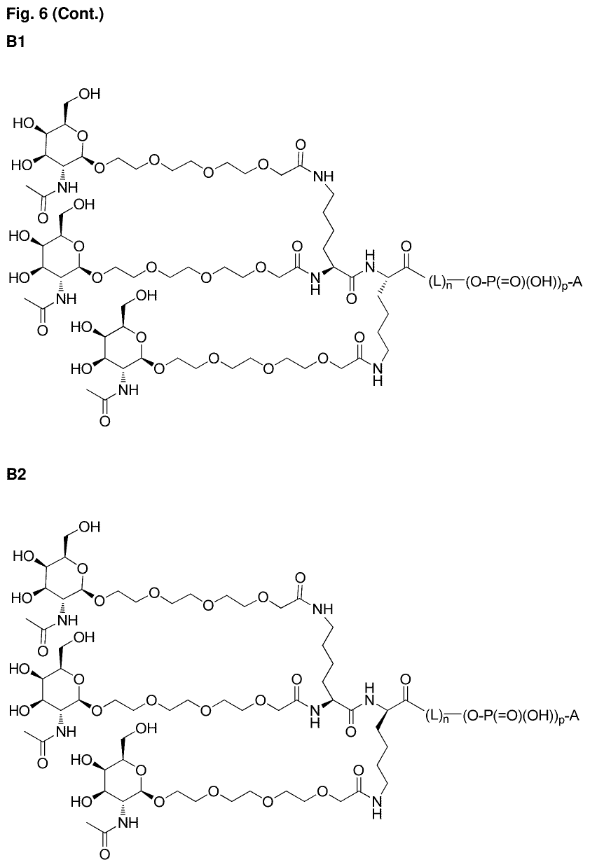

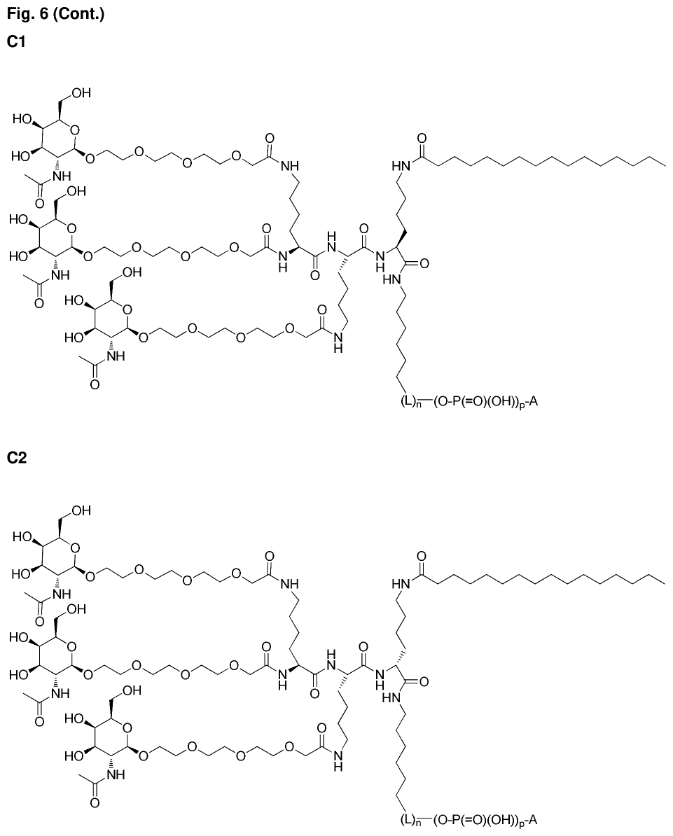

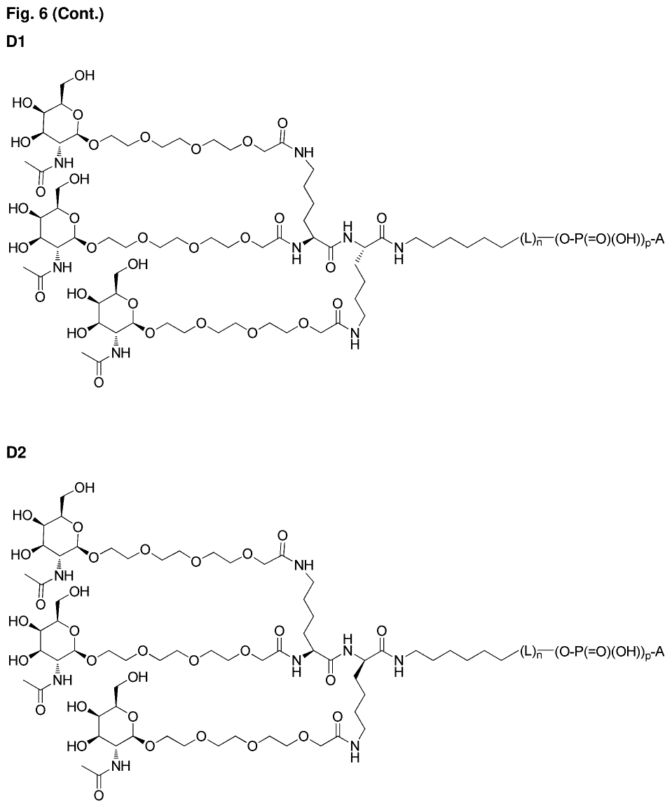

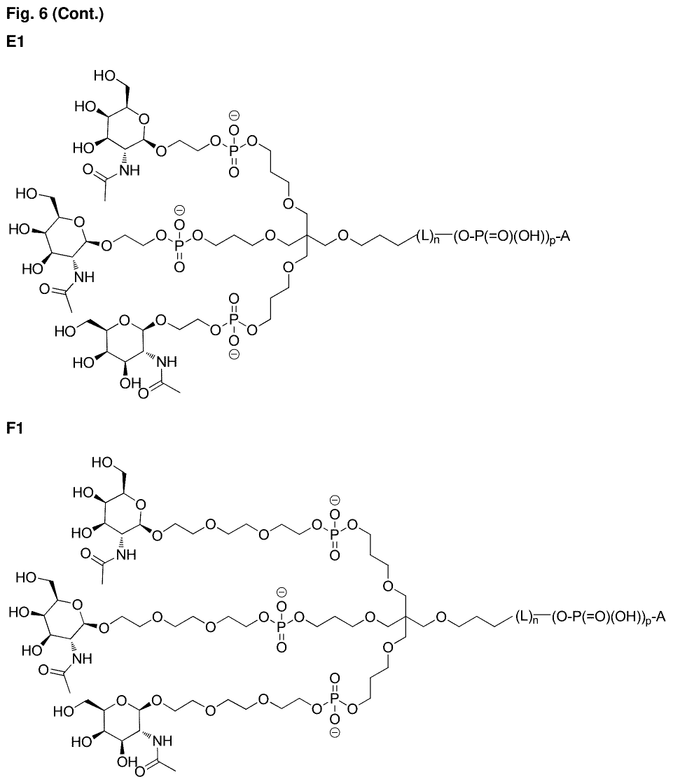

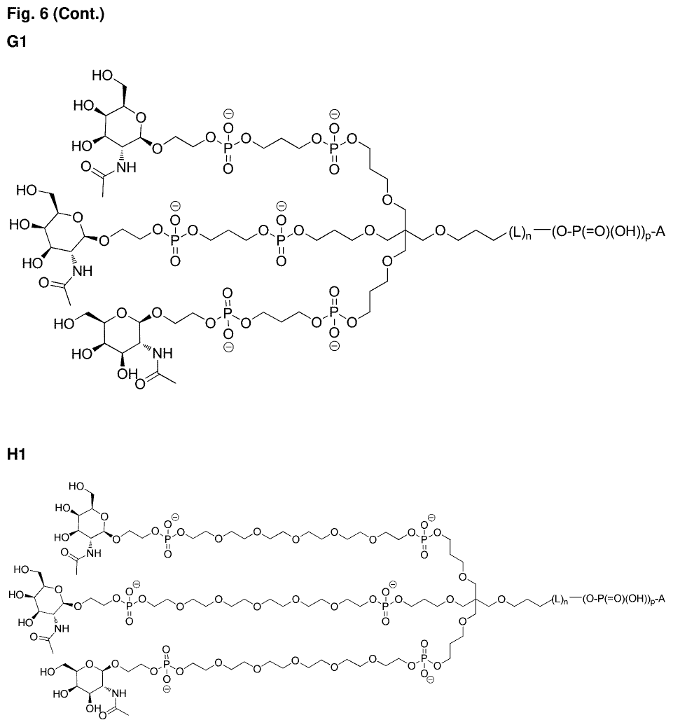

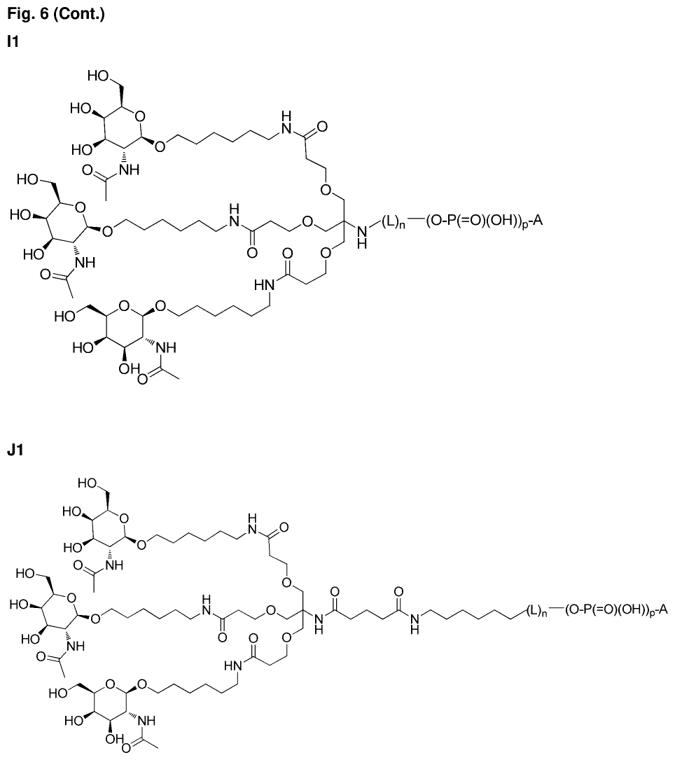

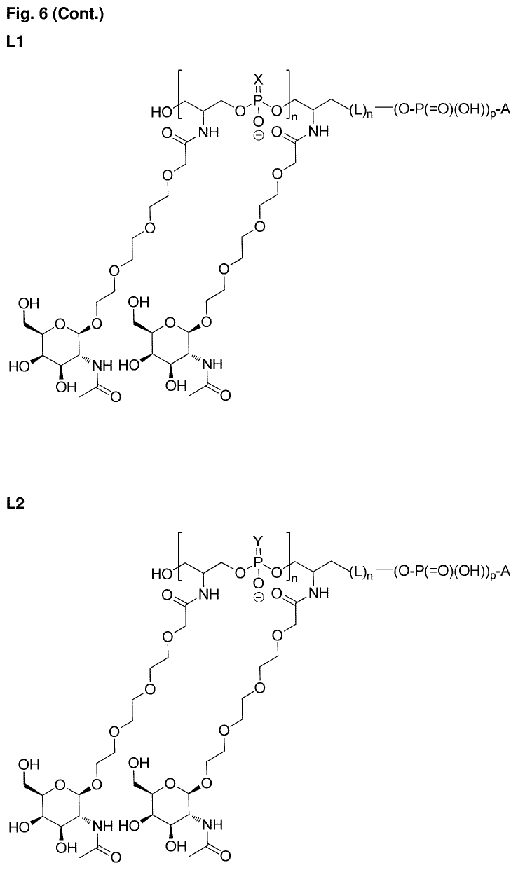

In one embodiment, the conjugate moiety is a tri-valent N-acetylgalactosamine (GalNAc), such as those shown in FIG. 5. In one embodiment, the conjugate moiety is the tri-valent N-acetylgalactosamine (GalNAc) of FIG. 5A-1 or FIG. 5A-2, or a mixture of both. In one embodiment, the conjugate moiety is the tri-valent N-acetylgalactosamine (GalNAc) of FIG. 5B-1 or FIG. 5B-2, or a mixture of both. In one embodiment, the conjugate moiety is the tri-valent N-acetylgalactosamine (GalNAc) of FIG. 5C-1 or FIG. 5C-2, or a mixture of both. In one embodiment, the conjugate moiety is the tri-valent N-acetylgalactosamine (GalNAc) of FIG. 5D-1 or FIG. 5D-2, or a mixture of both.

### RTEL1-Targeting Conjugate

In some embodiments, the conjugate targeting RTEL1 is selected from the group consisting of

5′-GN2-C6ₒ[X]AₛAₛTₛTₛtₛtₛaₛcₛaₛtₛaₛcₛtₛcₛtₛgₛGₛTₛ,
5′-GN2-C6ₒ
[X]AₛAₛtₛtₛtₛtₛaₛcₛaₛtₛaₛcₛtₛcₛtₛGₛGₛTₛ^{m}Cₛ,
5′-GN2-C6ₒ
[X]TₛTₛaₛcₛaₛtₛsₛaₛcₛtₛcₛtₛgₛgₛtₛ^{m}CₛAₛAₛAₛ,
5′-GN2-C6ₒ
[X]^{m}CₛTₛtₛtₛaₛtₛtₛaₛtₛaₛaₛcₛtₛTₛgₛaₛAₛtₛ^{m}CₛTₛ^{m}Cₛ;
and
5′-GN2-C6ₒ
[X]^{m}CₛTₛtₛtₛaₛtₛtₛaₛtₛaₛaₛcₛtₛtₛgₛaₛaₛTₛ^{m}CₛTₛ^{m}Cₛ.

wherein a capital letter represents a beta-D-oxy LNA nucleoside, a lower case letter represents a DNA nucleoside, wherein each LNA cytosine is 5-methyl cytosine, and wherein subscript s represents a phosphorothioate internucleoside linkage, and a subscript o represents a phosphodiester internucleoside linkage, and GN2-C6 is tri-valent N-acetylgalactosamine (GalNAc), such as those shown in FIG. 5, for example, such as the tri-valent N-acetylgalactosamine (GalNAc) of FIG. 5D-1 or FIG. 5D-2, or a mixture of both, and wherein and [X] represents cₒaₒ in accordance with the foregoing.

In some embodiments, the conjugate targeting RTEL1 is selected from the group of conjugates listed in Table 14, or a pharmaceutically acceptable salt thereof.

TABLE 14
Compound Table (Exemplary conjugates of the present invention) - HELM Annotation
Format (for the annotation on the HELM annotation, see explanations for Table 10).

| Oligo | | |
|---|---|---|
| Compound | | |
| ID | | |
| Number # | | |
| (acc. to | | Exemplary |
| Table 10 | HELM Annotation | compound - |
| above) | Written 5′ - 3′. | see FIG. |
| 243_1 | {[5gn2c6]P.[dR](C)P.[dR](A)P.[LR](A)[sP].[LR](A)[sP].[LR](T) | 1 |
| | [sP].[LR](T)[sP].[dR](T)[sP].[dR](T)[P].[dR](A)[P].[dR](C)[sP] | |
| | [dR](A)[sP].[dR](T)[sP].[dR](A)[sP].[dR](C)[sP].[dR](T)[sP].[d | |
| | R](C)[sP].[dR](T)[sP].[dR](G)[P].[LR](G)[sP].[LR](T) | |
| 244_1 | {[5gn2c6]P.[dR](C)P.[dR](A)P.[LR](A)[sP].[LR](A)[sP].[dR](T) | 2 |
| | [sP].[dR](T)[sP].[dR](T)[sP].[dR](T)[sP].[dR](A)[sP].[dR](C)[sP]. | |
| | [dR](A)[sP].[dR](T)[sP].[dR](A)[sP].[dR](C)[sP].[dR](T)[sP].[d | |
| | R](C)[sP].[dR](T)[sP].[LR](G)[sP].[LR](G)[sP].[LR](T)[sP].[LR] | |
| | ([5meC])} | |
| 245_1 | [5gn2c6]P.[dR](C)P.[dR](A)P.[LR](T)[sP].[LR](T)[sP].[dR](A)[s | 4 |
| | P].[dR](C)[sP].[dR](A)[sP].[dR](T)[sP].[dR](A)[sP].[dR](C)[sP]. | |
| | [dR](T)[sP].[dR](C)[P].[dR](T)[sP].[dR](G)[P].[dR](G)[sP].[d | |
| | R](T)[sP].[LR]([5meC])[sP].[LR](A)[sP].[LR](A)[sP].[LR](A) | |
| 246_1 | {[5gn2c6]P.[dR](C)P.[dR](A)P.[LR]([5meC])[sP].[LR](T)[sP].[d | 3 |
| | R](T)[sP].[dR](T)[sP].[dR](A)[sP].[dR](T)[sP].[dR](T)[sP].[dR] | |
| | (A)[sP].[dR](T)[sP].[dR](A)[sP].[dR](A)[sP].[dR](C)[sP].[dR](T) | |
| | [SP].[LR](T)[sP].[dR](G)[sP].[dR](A)[sP].[LR](A)[sP].[dR](T) | |
| | [sP].[LR]([5meC])[P].[LR](T)[sP].[LR]([5meC]) | |
| 246_2 | {[5gn2c6]P.[dR](C)P.[dR](A)P.[LR]([5meC])[sP].[LR](T)[sP].[d | |
| | R](T)[sP].[dR](T)[sP].[dR](A)[sP].[dR](T)[sP].[dR](T)[sP].[dR] | |
| | (A)[sP].[dR](T)[sP].[dR](A)[sP].[dR](A)[sP].[dR](C)[sP].[dR](T) | |
| | [sP].[dR](T)[sP].[dR](G)[sP].[dR](A)[sP].[dR](A)[sP].[LR](T) | |
| | [sP].[LR]([5meC])[sP].[LR](T)[sP].[LR]([5meC]) | |

### Helm Annotation Key:

[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC]) is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR](C) is a DNA cytosine nucleoside,
[sP] is a phosphorothioate internucleoside linkage,
P is a phosphodiester internucleoside linkage.
5gn2c6 is a trivalent N-acetylgalactosamine (GalNAc) of FIG. 5D-1 or FIG. 5D-2, or a mixture of both.

In some embodiments, 5gn2c6 is a GalNAc residue R having the formula:

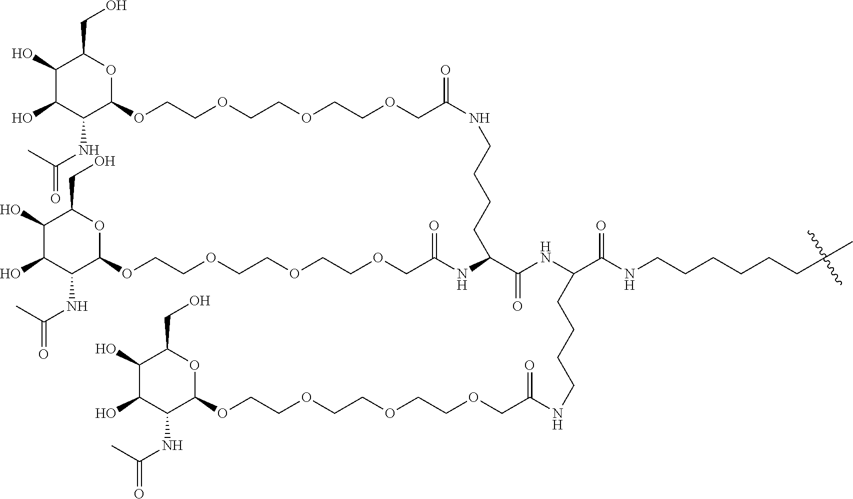 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

It is to be understood that R as shown in the figure above is a mixture of the two stereoisomers shown in FIGS. 5D1 and 5D2.

According to a further aspect of the invention, R as shown in the figure above is the stereoisomer as shown in to FIG. 5D1.

According to a further aspect of the invention R as shown in the figure above is the stereoisomer as shown in FIG. 5D2. The structures of the conjugates provided in Table 14 are shown in FIGS. 1 to 4.

The inhibitor may comprise the conjugate of FIG. 1, or a pharmaceutically acceptable salt thereof. The inhibitor may comprise the antisense oligonucleotide of Compound ID Number 243_1, or a pharmaceutically acceptable salt thereof.

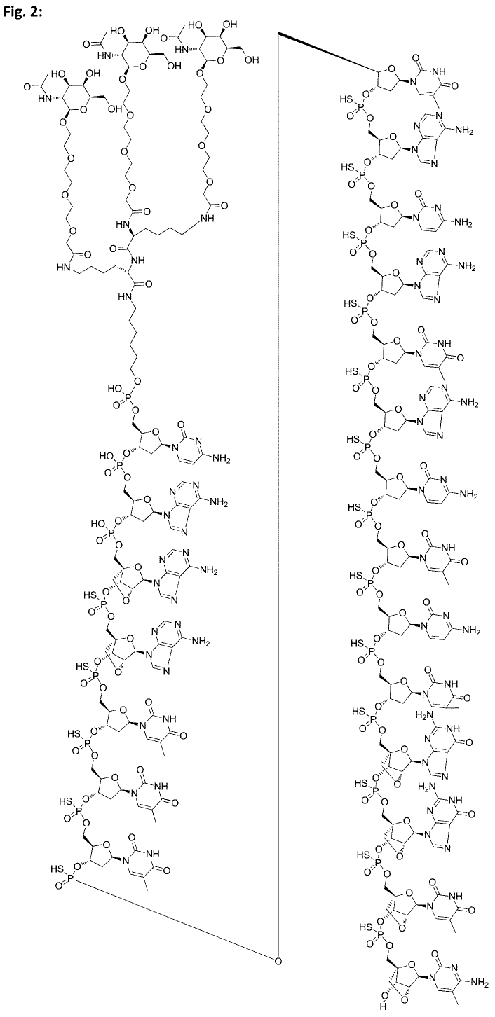

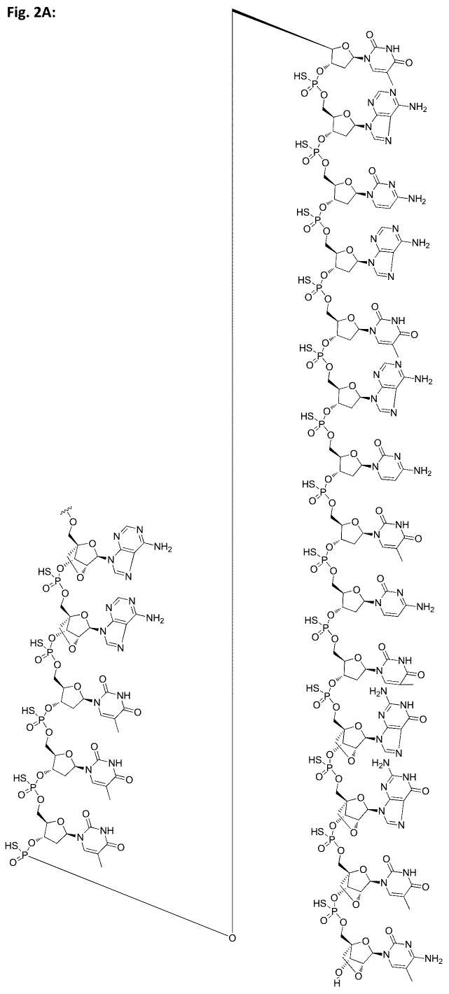

The inhibitor may comprise conjugate of FIG. 2, or a pharmaceutically acceptable salt thereof. The inhibitor may comprise the antisense oligonucleotide of Compound ID Number 244_1, or a pharmaceutically acceptable salt thereof.

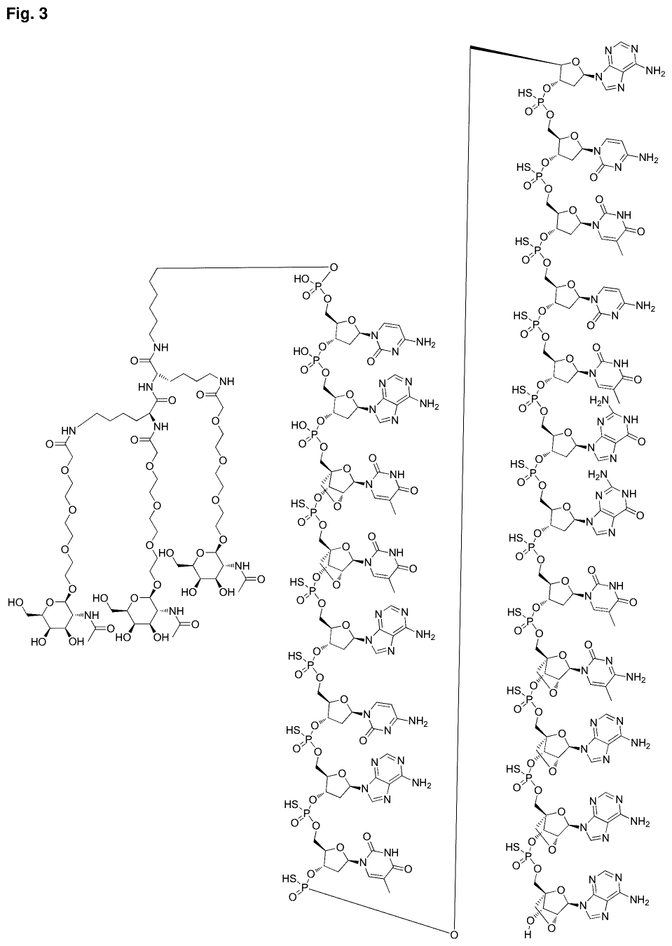

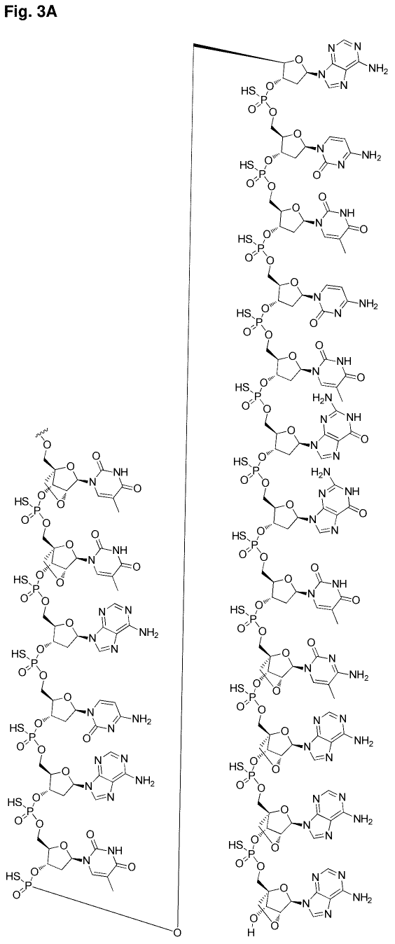

The inhibitor may comprise the conjugate of FIG. 3, or a pharmaceutically acceptable salt thereof. The inhibitor may comprise the antisense oligonucleotide of Compound ID Number 245_1, or a pharmaceutically acceptable salt thereof.

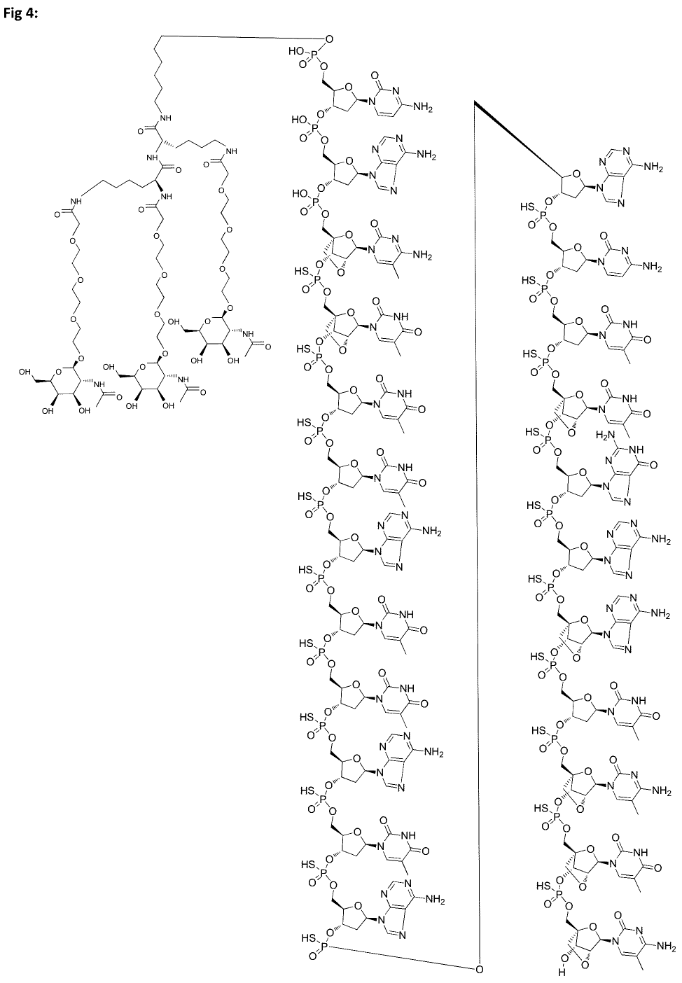

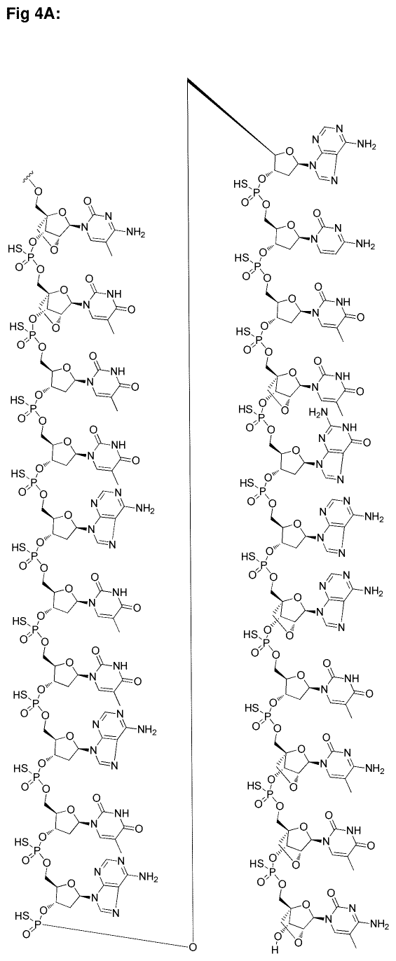

The inhibitor may comprise the conjugate of FIG. 4, or a pharmaceutically acceptable salt thereof. The inhibitor may comprise the antisense oligonucleotide of Compound ID Number 246_1, or a pharmaceutically acceptable salt thereof.

Chemical drawings representing some of the conjugate of the combination of the invention are shown in FIGS. 1 to 4.

In some embodiments, the conjugate is the conjugate as shown in FIG. 1.

In some embodiments, the conjugate is the conjugate as shown in FIG. 2.

In some embodiments, the conjugate is the conjugate as shown in FIG. 3.

In some embodiments, the conjugate is the conjugate as shown in FIG. 4.

The compounds illustrated in FIGS. 1-4 are shown in the protonated form—the S atom on the phosphorothioate linkage is protonated—it will be understood that the presence of the proton will depend on the acidity of the environment of the molecule, and the presence of an alternative cation (e.g. when the oligonucleotide is in salt form). Protonated phosphorothioates exist in tautomeric forms.

### FUBP1-Targeting Conjugate

In some embodiments, the conjugate targeting FUBP1 is selected from the group consisting of

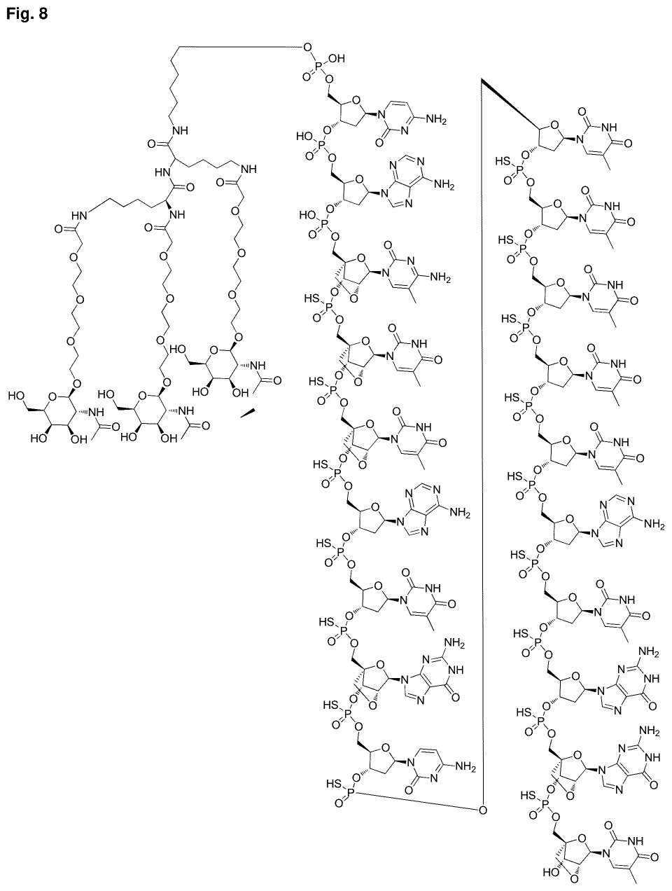

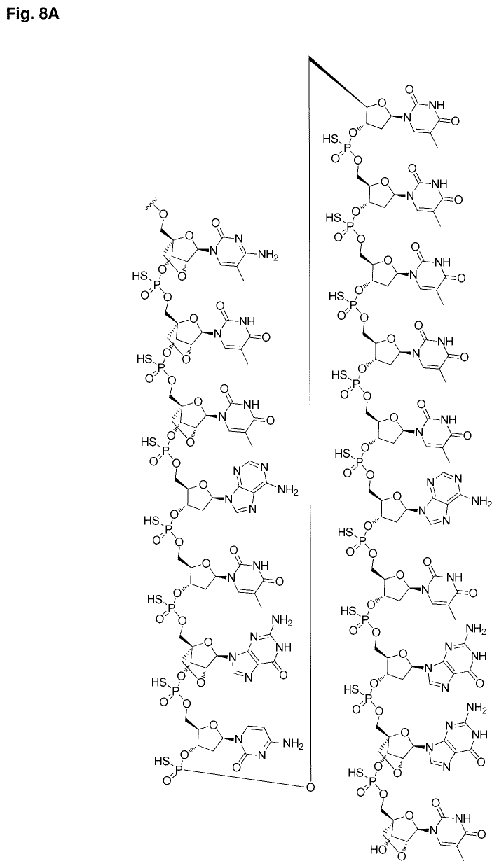

5′-GN2-C6ₒ[X]^{m}CₛTₛTₛaₛtₛGₛcₛtₛtₛtₛtₛtₛaₛtₛgₛGₛT,
5′-GN2-C6ₒ[X]^{m}CₛTₛTₛaₛTₛgₛcₛtₛtₛtₛtₛtₛaₛtₛgₛGₛT,
5′-GN2-C6ₒ[X]^{m}CₛTₛtₛAₛTₛgₛcₛtₛtₛtₛtₛtₛaₛtₛgₛGₛTₛT,
5′-GN2-C6ₒ[X]^{m}CₛTₛtₛAₛtₛgₛcₛtₛtₛtₛtₛtₛaₛtₛgₛGₛTₛT,
5′-GN2-C6ₒ[X]^{m}CₛTₛtₛAₛtₛgₛcₛtₛtₛtₛtₛtₛaₛtₛGₛgₛTₛT,
5′-GN2-C6ₒ[X]^{m}CₛTₛtₛAₛtₛgₛcₛtₛtₛtₛtₛtₛaₛtₛGₛGₛTₛT,
5′-GN2-C6ₒ[X]GₛcₛtₛtₛTₛtₛtₛaₛtₛgₛgₛtₛTₛtₛ^{m}CₛAₛ^{m}C,
5′-GN2-C6ₒ[X]TₛAₛTₛgₛcₛTₛtₛtₛtₛtₛaₛtₛgₛgₛtₛTₛTₛ^{m}C,
5′-GN2-
C6ₒ[X]Aₛcₛ^{m}CₛAₛAₛtₛtₛtₛtₛcₛaₛtₛtₛtₛ^{m}CₛtAₛ^{m}C,
and
5′-GN2-C6ₒ[X]^{m}CₛcₛcₛcₛaₛtₛaₛaₛcₛcₛaₛtₛaₛGₛTₛ^{m}Cₛ

wherein a capital letter represents a beta-D-oxy LNA nucleoside, a lower case letter represents a DNA nucleoside, wherein each LNA cytosine is 5-methyl cytosine, and wherein subscript s represents a phosphorothioate internucleoside linkage, and a subscript o represents a phosphodiester internucleoside linkage, and GN2-C6 is tri-valent N-acetylgalactosamine (GalNAc) as shown in FIG. 5D, such as a tri-valent N-acetylgalactosamine (GalNAc) as shown in FIG. 5D-1 or FIG. 5D2, or a mixture of both, preferably bound via a phosphodiester linkage at the 5′ end of the oligonucleotide. Chemical drawings representing some of the molecules are shown in FIGS. 8 to 16, and wherein and [X] represents cₒaₒ in accordance with the foregoing.

In some embodiments, the conjugate targeting FUBP1 is selected from the group of conjugates listed in Table 15, or a pharmaceutically acceptable salt thereof.

TABLE 15A
Compound Table (Exemplary conjugates of the present invention) - HELM
Annotation Format.

| Oligo | | |
|---|---|---|
| Compound | | |
| ID | | |
| Number # | | |
| (acc. to | | Exemplary |
| Table 13 | HELM Annotation | compound - |
| above) | Written 5′ - 3′. | see FIG. |
| 325_1 | {[5gn2c6]P.[dR](C)P.[dR](A)P.[LR]([5meC])[sP].[LR](T)[sP].[L | 8 |
| | R](T)[sP].[dR](A)[sP].[dR](T)[sP].[LR](G)[sP].[dR](C)[sP].[dR] | |
| | (T)[sP].[dR](T)[sP].[dR](T)[sP].[dR](T)[sP].[dR](T)[sP].[dR](A) | |
| | [sP].[dR](T)[sP].[dR](G)[P].[LR](G)[sP].[LR](T)} | |
| 325_2 | {[5gn2c6]P.[dR](C)P.[dR](A)P.[LR]([5meC])[sP].[LR](T)[sP].[L | 9 |
| | R](T)[sP].[dR](A)[sP].[LR](T)[sP].[dR](G)[P].[dR](C)[sP].[dR] | |
| | (T)[sP].[dR](T)[sP].[dR](T)[sP].[dR](T)[sP].[dR](T)[sP].[dR](A) | |
| | [sP].[dR](T)[sP].[dR](G)[sP].[LR](G)[sP].[LR](T)} | |
| 326_1 | {[5gn2c6]P.[dR](C)P.[dR](A)P.[LR]([5meC])[sP].[LR](T)[P].[d | 10 |
| | R](T)[sP].[LR](A)[sP].[LR](T)[sP].[dR](G)[sP].[dR](C)[sP].[dR] | |
| | (T)[sP].[dR](T)[sP].[dR](T)[sP].[dR](T)[sP].[dR](T)[sP].[dR](A) | |
| | [sP].[dR](T)[sP].[dR](G)[sP].[LR](G)[sP].[LR](T)[sP].[LR](T)} | |
| 326_2 | {[5gn2c6]P.[dR](C)P.[dR](A)P.[LR]([5meC])[sP].[LR](T)[sP].[d | 11 |
| | RI(T)[sP].[LR](A)[sP].[dR](T)[sP].[dR](G)[sP].[dR](C)[sP].[dR] | |
| | (T)[sP].[dR](T)[sP].[dR](T)[sP].[dR](T)[sP].[dR](T)[sP].[dR](A) | |
| | [sP].[dR](T)[sP].[dR](G)[sP].[LR](G)[sP].[LR](T)[sP].[LR](T)} | |
| 326_3 | {[5gn2c6]P.[dR](C)P.[dR](A)P.[LR]([5meC])[sP].[LR](T)[sP].[d | 12 |
| | R](T)[sP].[LR](A)[sP].[dR](T)[sP].[dR](G)[sP].[dR](C)[sP].[dR] | |
| | (T)[sP].[dR](T)[sP].[dR](T)[sP].[dR](T)[sP].[dR](T)[sP].[dR](A) | |
| | [sP].[dR](T)[sP].[LR](G)[sP].[dR](G)[sP].[LR](T)[sP].[LR](T)} | |
| 326_4 | {[5gn2c6]P.[dR](C)P.[dR](A)P.[LR]([5meC])[sP].[LR](T)[sP].[d | 13 |
| | R](T)[sP].[LR](A)[sP].[dR](T)[sP].[dR](G)[sP].[dR](C)[sP].[dR] | |
| | (T)[sP].[dR](T)[sP].[dR](T)[sP].[dR](T)[sP].[dR](T)[sP].[dR](A) | |
| | [sP].[dR](T)[sP].[LR](G)[sP].[LR](G)[sP].[LR](T)[sP].[LR](T)} | |
| 327_1 | {[5gn2c6]P.[dR](C)P.[dR](A)P.[LR](G)[sP].[dR](C)[sP].[dR](T) | 14 |
| | [sP].[dR](T)[sP].[LR](T)[sP].[dR](T)[sP].[dR](T)[sP].[dR](A)[sP]. | |
| | [dR](T)[sP].[dR](G)[sP].[dR](G)[sP].[dR](T)[sP].[LR](T)[sP]. | |
| | [dR](T)[sP].[LR]([5meC])[sP].[LR](A)[sP].[LR]([5meC])} | |
| 328_1 | {[5gn2c6]P.[dR](C)P.[dR](A)P.[LR](T)[sP].[LR](A)[P].[LR](T) | 15 |
| | [sP].[dR](G)[sP].[dR](C)[sP].[LR](T)[sP].[dR](T)[sP].[dR](T)[sP]. | |
| | [dR](T)[sP].[dR](T)[sP].[dR](A)[sP].[dR](T)[sP].[dR](G)[sP]. | |
| | [dR](G)[sP].[dR](T)[sP].[LR](T)[sP].[LR](T)[sP].[LR]([5meC])} | |
| 329_1 | {[5gn2c6]}P.[dR](C)P.[dR](A)P.[LR](A)[sP].[dR](C)[P].[LR]([5meC]) | 16 |
| | [sP].[LR](A)[sP].[LR](A)[sP].[dR](T)[sP].[dR](T)[sP].[dR] | |
| | (T)[sP].[dR](T)[sP].[dR](C)[sP].[dR](A)[sP].[dR](T)[sP].[dR](T) | |
| | [sP].[dR](T)[sP].[LR]([5meC])[sP].[dR](T)[sP].[LR](A)[sP].[LR] | |
| | ([5meC])} | |
| 330_1 | {[5gn2c6]}P.[dR](C)P.[dR](A)P.[LR]([5meC])[sP].[dR](C)[sP]. | |
| | [dR](C)[sP].[dR](C)[sP].[dR](A)[P].[dR](T)[sP].[dR](A)[sP].[d | |
| | R](A)[sP].[dR](C)[sP].[dR](C)[sP].[dR](A)[sP].[dR](T)[sP].[dR] | |
| | (A)[sP].[LR](G)[sP].[LR](T)[sP].[LR]([5meC])} | |

In the above Table, [5gn2c6] is a GalNAc residue R having the formula:

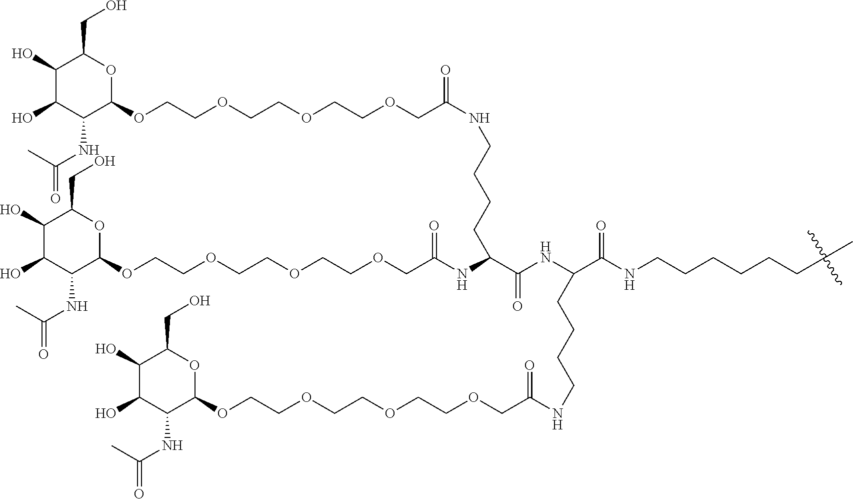 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

It is to be understood that R as shown in the figure above and as used in the above table is a mixture of the two stereoisomers shown in FIGS. 5D1 and 5D2.

According to a further aspect of the invention, R as shown in the figure above and as used in the above table is the stereoisomer as shown in to FIG. 5D1.

According to a further aspect of the invention R as shown in the figure above and as used in the above table is the stereoisomer as shown in FIG. 5D1. The structures of the conjugates provided in Table 15 are shown in FIGS. 8 to 16.

The invention provides for the conjugate of FIG. 8, or a pharmaceutically acceptable salt thereof.

The invention provides for the antisense oligonucleotide of Compound ID Number 325_1, or a pharmaceutically acceptable salt thereof.

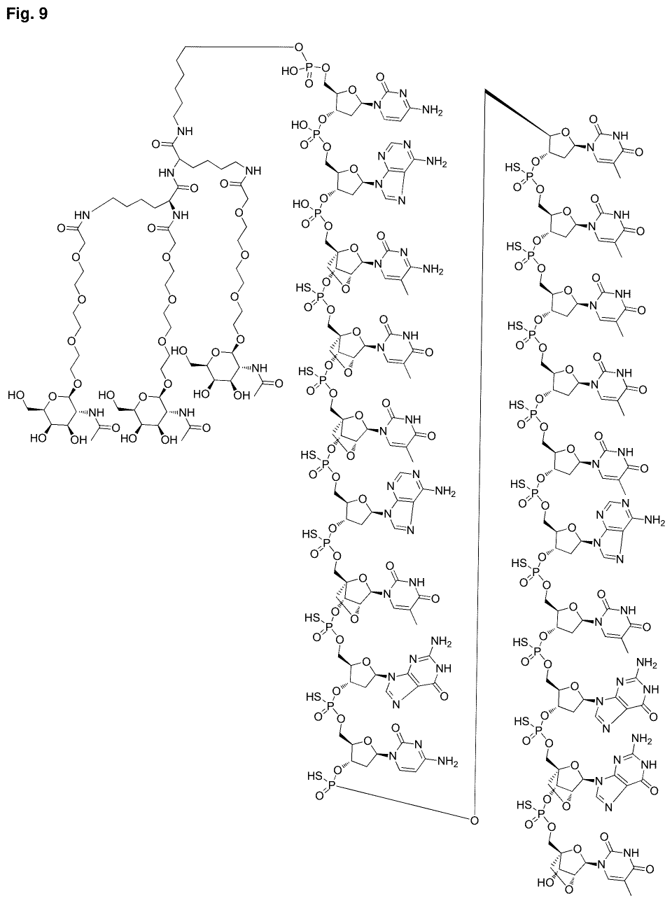

The invention provides for the conjugate of FIG. 9, or a pharmaceutically acceptable salt thereof.

The invention provides for the antisense oligonucleotide of Compound ID Number 325_2, or a pharmaceutically acceptable salt thereof.

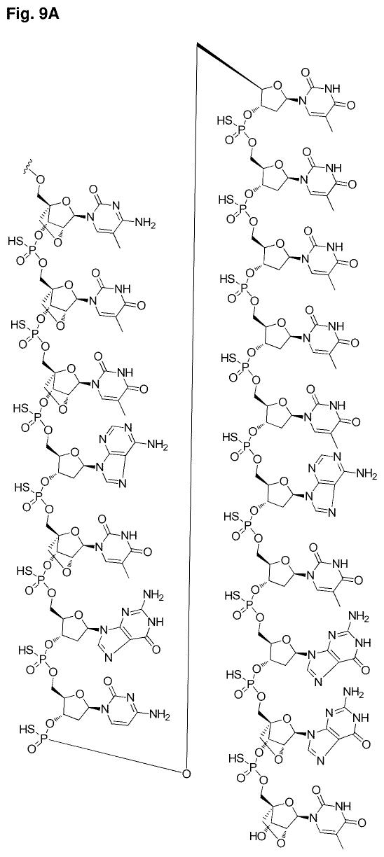

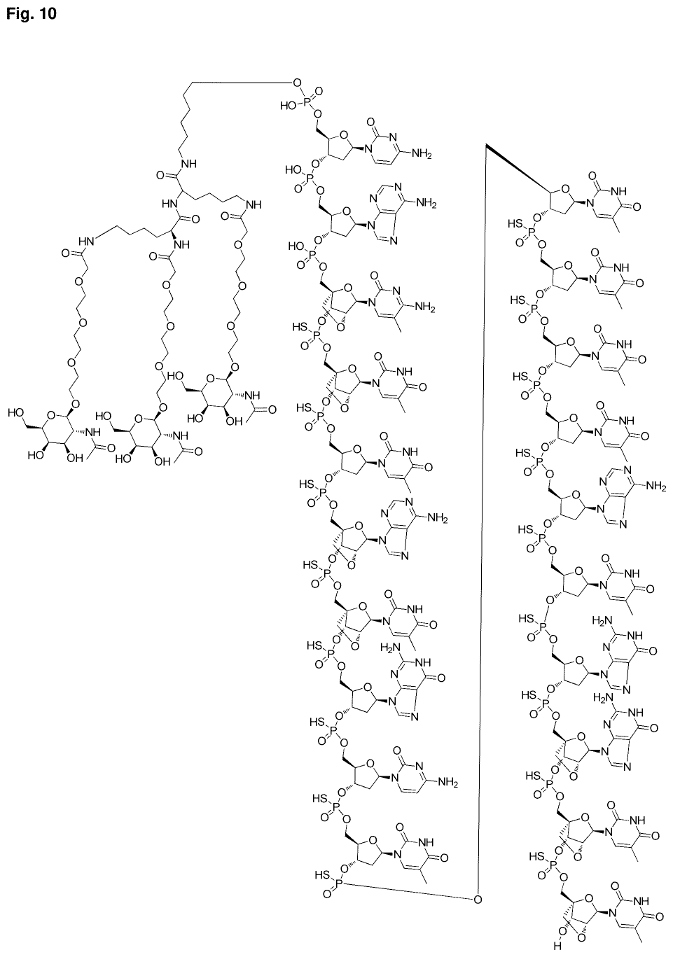

The invention provides for the conjugate of FIG. 10, or a pharmaceutically acceptable salt thereof.

The invention provides for the antisense oligonucleotide of Compound ID Number 326_1, or a pharmaceutically acceptable salt thereof.

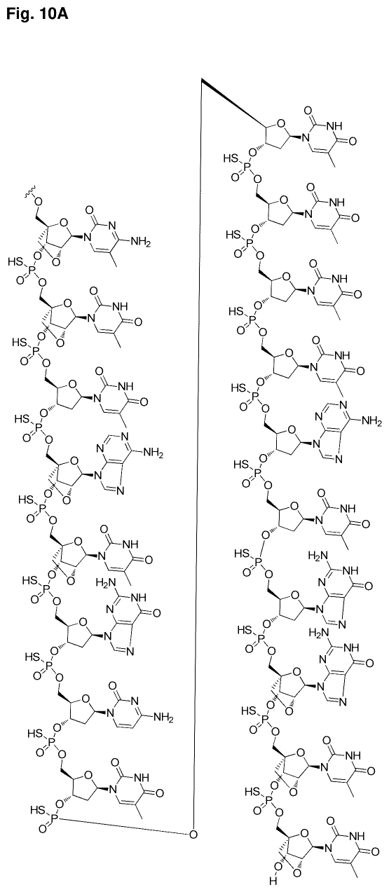

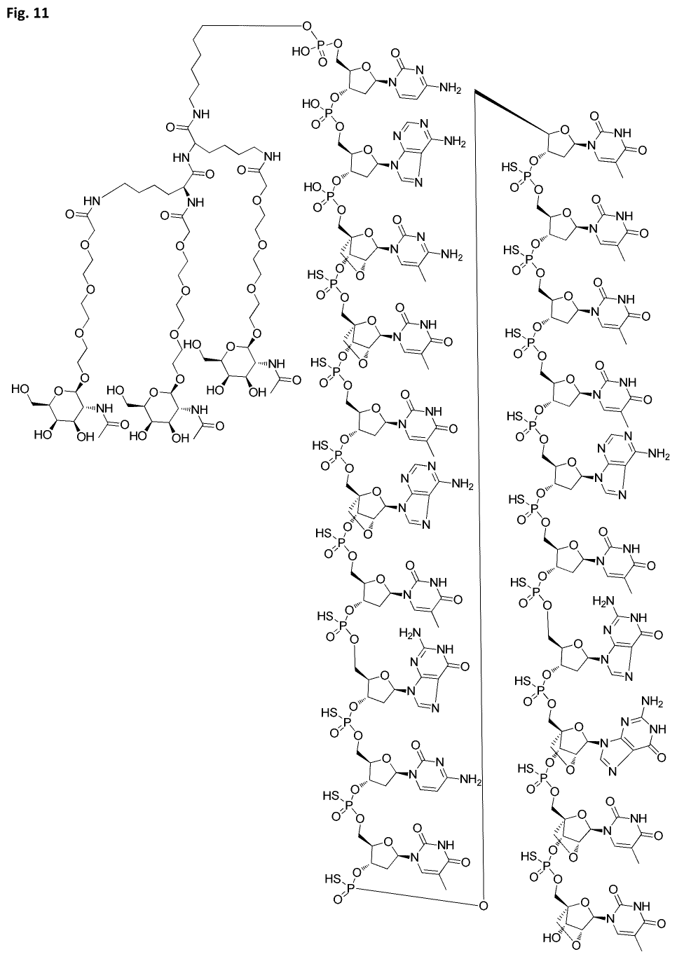

The invention provides for the conjugate of FIG. 11, or a pharmaceutically acceptable salt thereof.

The invention provides for the antisense oligonucleotide of Compound ID Number 326_2, or a pharmaceutically acceptable salt thereof.

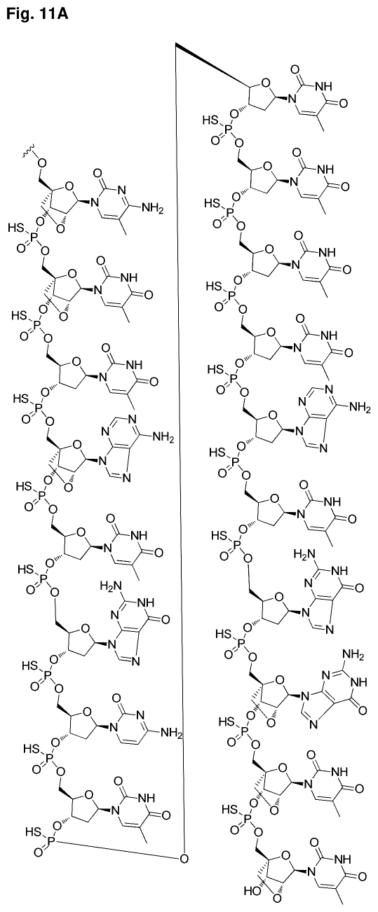

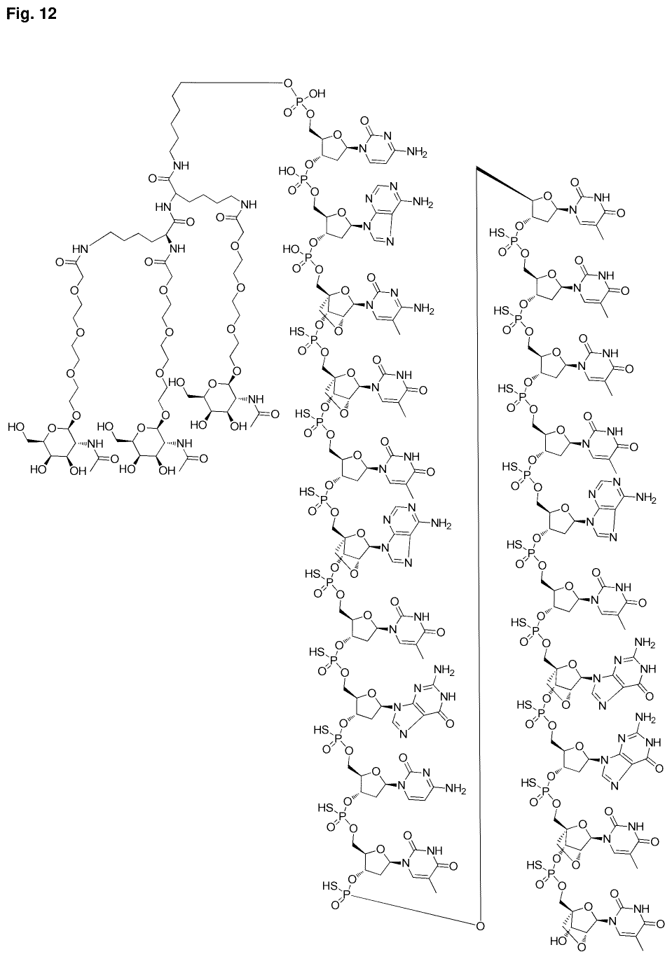

The invention provides for the conjugate of FIG. 12, or a pharmaceutically acceptable salt thereof.

The invention provides for the antisense oligonucleotide of Compound ID Number 326_3, or a pharmaceutically acceptable salt thereof.

The invention provides for the conjugate of FIG. 13, or a pharmaceutically acceptable salt thereof.

The invention provides for the antisense oligonucleotide of Compound ID Number 326_4, or a pharmaceutically acceptable salt thereof.

The invention provides for the conjugate of FIG. 14, or a pharmaceutically acceptable salt thereof.

The invention provides for the antisense oligonucleotide of Compound ID Number 327_1, or a pharmaceutically acceptable salt thereof.

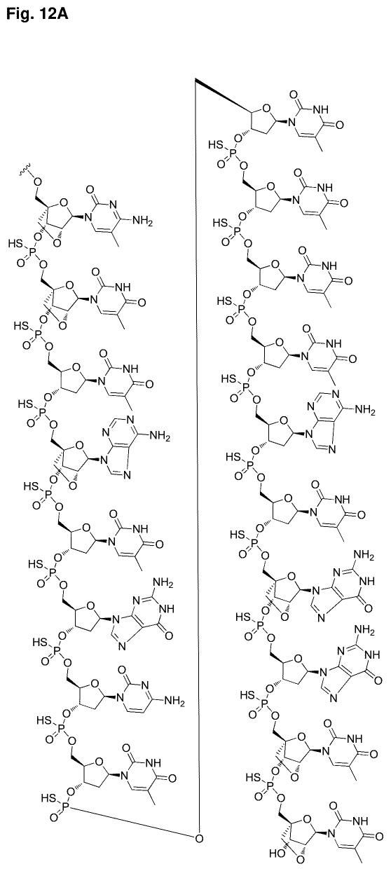

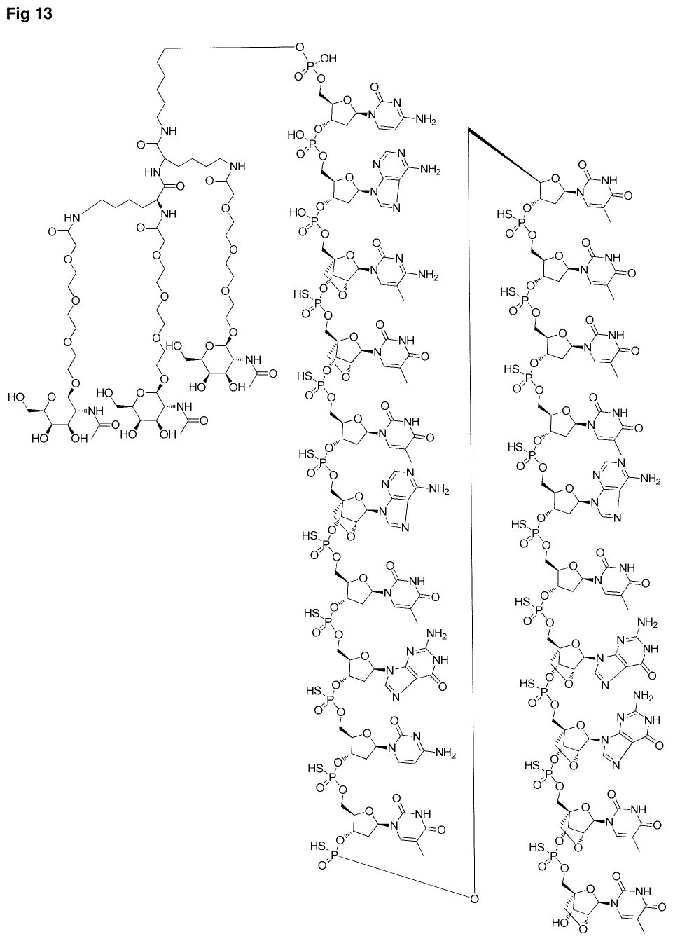

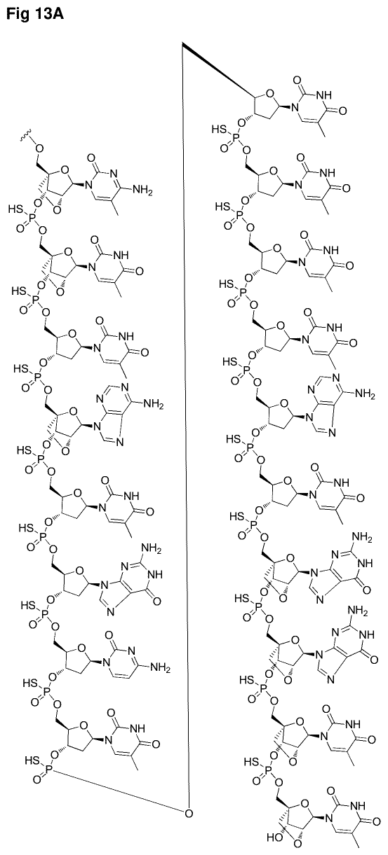

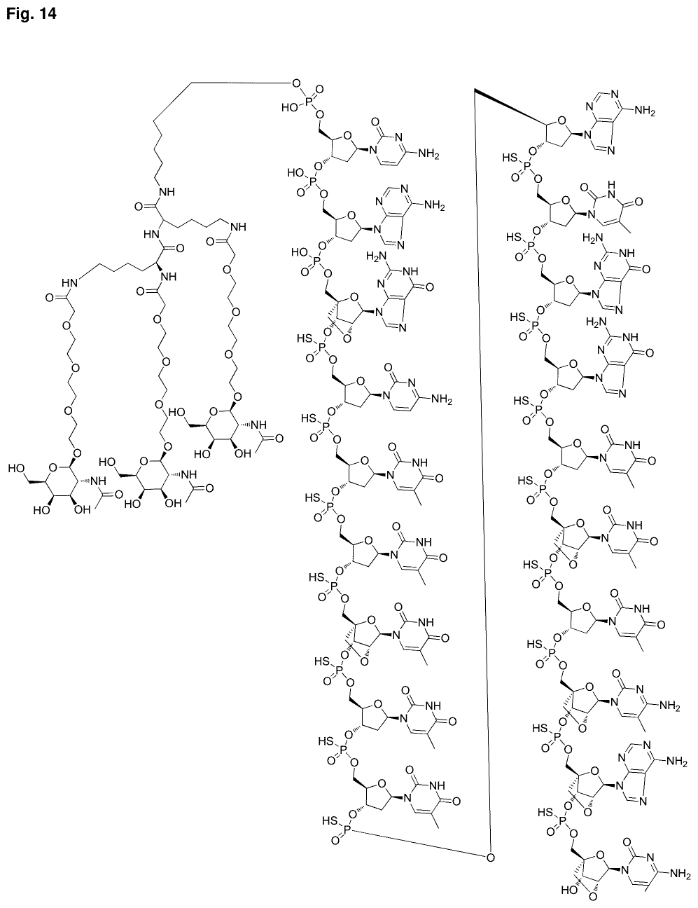

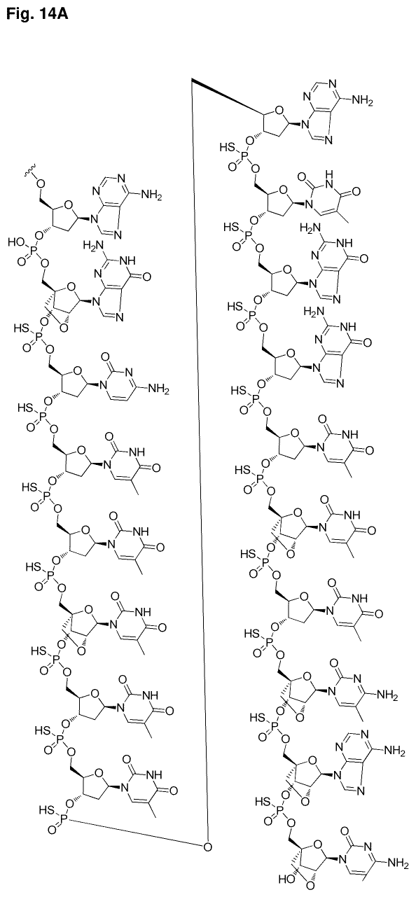

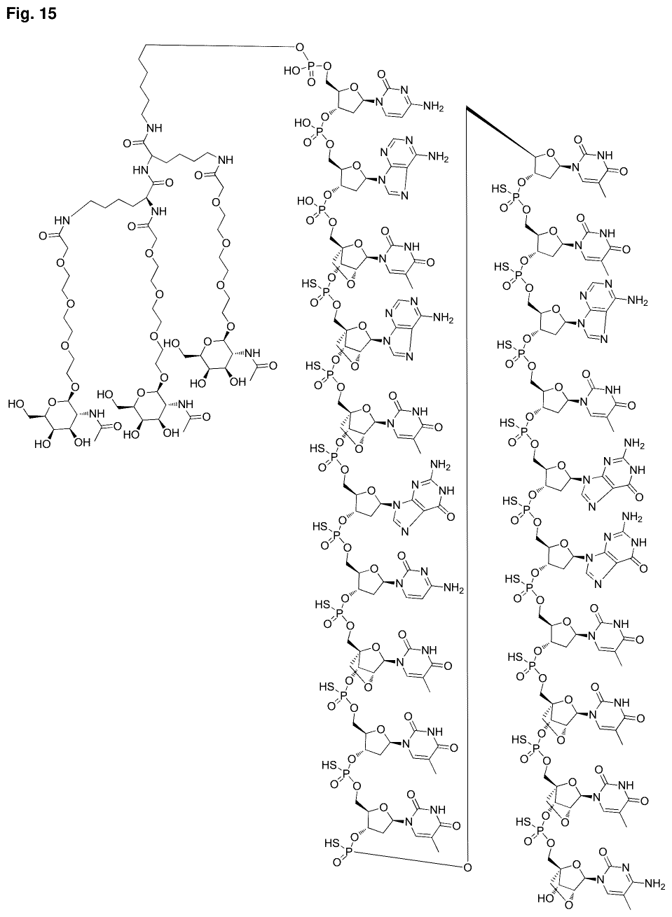

The invention provides for the conjugate of FIG. 15, or a pharmaceutically acceptable salt thereof.

The invention provides for the antisense oligonucleotide of Compound ID Number 328_1, or a pharmaceutically acceptable salt thereof.

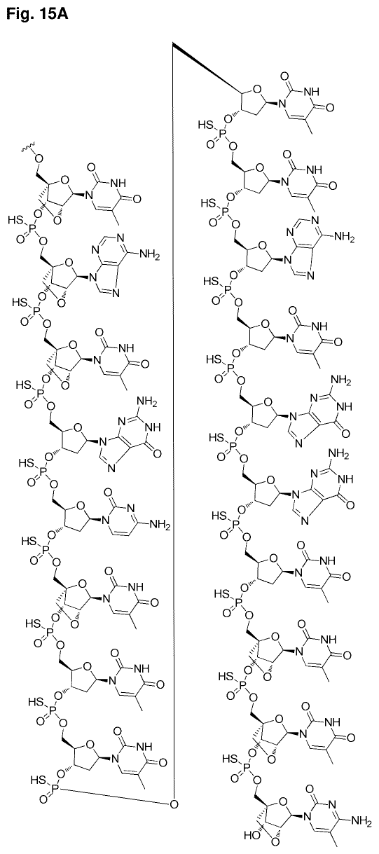

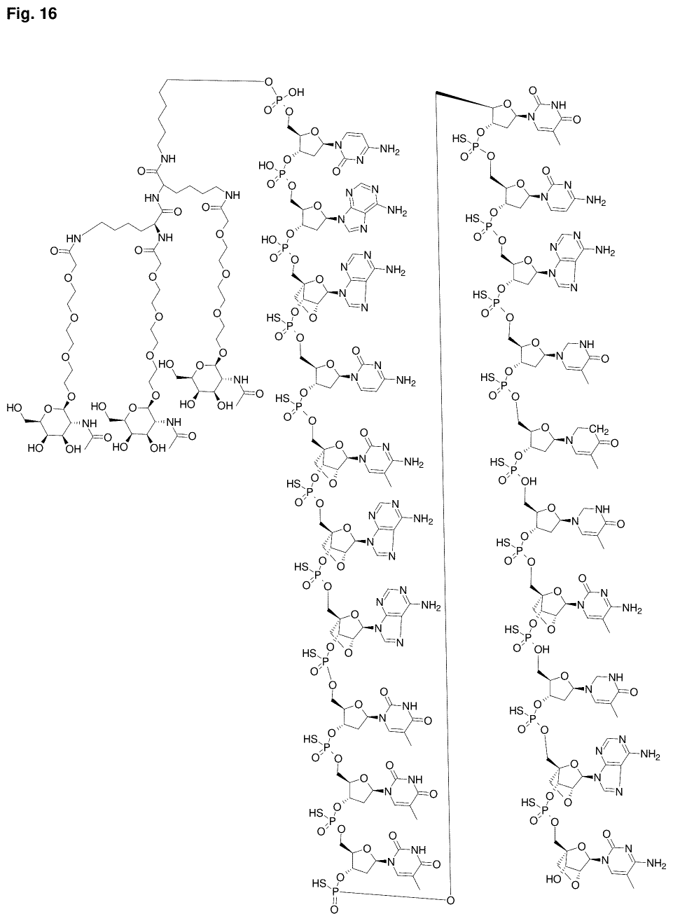

The invention provides for the conjugate of FIG. 16, or a pharmaceutically acceptable salt thereof.

The invention provides for the antisense oligonucleotide of Compound ID Number 329_1, or a pharmaceutically acceptable salt thereof.

The invention provides for the antisense oligonucleotide of Compound ID Number 330_1, or a pharmaceutically acceptable salt thereof.

In some embodiments, the conjugate is the conjugate as shown in FIG. 8.

In some embodiments, the conjugate is the conjugate as shown in FIG. 9.

In some embodiments, the conjugate is the conjugate as shown in FIG. 10.

In some embodiments, the conjugate is the conjugate as shown in FIG. 11.

In some embodiments, the conjugate is the conjugate as shown in FIG. 12.

In some embodiments, the conjugate is the conjugate as shown in FIG. 13.

In some embodiments, the conjugate is the conjugate as shown in FIG. 14.

In some embodiments, the conjugate is the conjugate as shown in FIG. 15.

In some embodiments, the conjugate is the conjugate as shown in FIG. 16

The compounds illustrated in FIGS. 8-16 are shown in the protonated form—the S atom on the phosphorothioate linkage is protonated—it will be understood that the presence of the proton will depend on the acidity of the environment of the molecule, and the presence of an alternative cation (e.g. when the oligonucleotide is in salt form). Protonated phosphorothioates exist in tautomeric forms.

### Combination-Targeting Conjugate

In some embodiments, the conjugate of the combination of compounds targeting RTEL1 and FUBP1 is selected from the group consisting of

(CMP ID NO 350_1)
5′-GN2-C6ₒ[X]^{m}CₛTₛtₛAₛtₛgₛcₛtₛtₛtₛtₛtₛaₛtₛGₛgₛTₛTₒcₒaₒ
TₛTₛaₛcₛaₛtₛaₛcₛtₛcₛtₛgₛgₛtₛ^{m}CₛAₛAₛ,
and
(CMP ID NO 351_1)
5′-GN2-C6ₒ[X]TₛTₛaₛcₛaₛtₛaₛcₛtₛcₛtₛgₛgₛtₛ^{m}CₛAₛAₛAₒcₒaₒ
^{m}CₛTₛtₛAₛtₛgₛcₛtₛtₛtₛtₛtₛaₛtₛGₛgₛTₛT

wherein a capital letter represents a beta-D-oxy LNA nucleoside, a lower case letter represents a DNA nucleoside, wherein each LNA cytosine is 5-methyl cytosine, and wherein subscript s represents a phosphorothioate internucleoside linkage, and a subscript o represents a phosphodiester internucleoside linkage, and GN2-C6 is tri-valent N-acetylgalactosamine (GalNAc) as shown in FIG. 5D, such as a tri-valent N-acetylgalactosamine (GalNAc) as shown in FIG. 5D-1 or FIG. 5D2, or a mixture of both, preferably bound via a phosphodiester linkage at the 5′ end of the 5′ most oligonucleotide, and wherein and [X] represents cₒaₒ in accordance with the foregoing.

TABLE 15B
Combination of Compound Table (Exemplary conjugates of the present invention)-
HELM Annotation Format

| Oligo | |
|---|---|
| Compound | |
| ID | HELM Annotation (Written 5′ - 3′.) |
| 350_1 | CHEM 1{[5gn2c6]}\|RNA1{P.[dR](C)P.[dR](A)P.[LR]([5meC])[sP].[LR](T)[sP].[dR] |
| | (T)[sP].[LR](A)[sP].[dR](T)[P].[dR](G)[sP].[dR](C)[sP].[dR](T)[sP].[dR](T)[sP]. |
| | [dR](T)[sP].[dR](T)[sP].[dR](T)[sP].[dR](A)[sP].[dR](T)[sP].[LR](G)[sP].[dR] |
| | (G)[sP].[LR](T)[sP].[LR](T)P.[dR](C)P.[dR](A)P.[LR](T)[sP].[LR](T)[P].[dR](A) |
| | [sP].[dR](C)[sP].[dR](A)[sP].[dR](T)[sP].[dR](A)[sP].[dR](C)[sP].[dR](T)[sP]. |
| | [dR](C)[sP].[dR](T)[sP].[dR](G)[sP].[dR](G)[sP].[dR](T)[sP].[LR]([5meC])[sP]. |
| | [LR](A)[sP].[LR](A)[sP].[LR](A)}$CHEM1, RNA1, 1:R2-1:R1$$$V2.0 |
| 351_1 | CHEM 1{[5gn2c6]}\|RNA1{P.[dR](C)P.[dR](A)P.[LR](T)[sP].[LR](T)[sP].[dR](A)[sP]. |
| | [dR](C)[sP].[dR](A)[sP].[dR](T)[sP].[dR](A)[sP].[dR](C)[sP].[dR](T)[sP].[dR] |
| | (C)[sP].[dR](T)[sP].[dR](G)[sP].[dR](G)[sP].[dR](T)[sP].[LR]([5meC])[sP].[LR] |
| | (A)[sP].[LR](A)[sP].[LR](A)P.[dR](C)P.[dR](A)P.[LR]([5meC])[sP].[LR](T)[sP]. |
| | [dR](T)[sP].[LR](A)[sP].[dR](T)[sP].[dR](G)[sP].[dR](C)[sP].[dR](T)[sP].[dR] |
| | (T)[sP].[dR](T)[sP].[dR](T)[sP].[dR](T)[sP].[dR](A)[sP].[dR](T)[sP].[LR](G)[sP]. |
| | [dR](G)[sP].[LR](T)[sP].[LR](T)}$CHEM1, RNA1, 1:R2-1:R1$$$V2.0 |

### Method of Manufacture

In a further aspect, methods for manufacturing the oligonucleotides of the combination of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment, the method further comprises reacting the contiguous nucleotide sequence with a conjugating moiety (ligand) to covalently attach the conjugate moiety to the oligonucleotide. In a further aspect a method is provided for manufacturing the composition of the combination of the invention, comprising mixing the oligonucleotide or conjugated oligonucleotide of the combination of the invention with a pharmaceutically acceptable diluent, solvent, carrier, salt and/or adjuvant.

### Pharmaceutical Salt

The compounds according to the present invention may exist in the form of their pharmaceutically acceptable salts. The terms “pharmaceutical salt” or “pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin, Organic Process Research & Development 2000, 4, 427-435 or in Ansel, In: Pharmaceutical Dosage Forms and Drug Delivery Systems, 6th ed. (1995), pp. 196 and 1456-1457. For example, the pharmaceutically acceptable salt of the compounds provided herein may be a sodium salt.

In a further aspect the invention relates to a pharmaceutically acceptable salt of one or more of the antisense oligonucleotide or a conjugate thereof, such as a pharmaceutically acceptable sodium salt, ammonium salt or potassium salt.

### Pharmaceutical Combinations and Kits

One aspect of present invention relates to a pharmaceutical combination of an inhibitor targeting RTEL1 and an inhibitor of FUBP1 as described herein, each formulated in a pharmaceutically acceptable carrier.

The pharmaceutical combination of the present invention can be used to treat an HBV infection more effectively than the comprised therapeutic inhibitors, such as oligonucleotides, taken alone. In an embodiment, the pharmaceutical combination of the present invention can be used to inhibit HBV more rapidly, to inhibit HBV with an increased duration and/or to inhibit HBV with greater effect than the comprised therapeutic inhibitors, such as oligonucleotide, taken alone. These effects may be measured by a reduction in cccDNA in an infected cell. In an embodiment, the pharmaceutical combination of the present invention causes a more rapid reduction in cccDNA in an infected cell than the comprised therapeutic inhibitors, such as oligonucleotide, taken alone. In an embodiment, the pharmaceutical combination of the present invention causes a more prolonged reduction in cccDNA than the comprised therapeutic oligonucleotide or TLR7 agonist alone. In an embodiment, the pharmaceutical combination of the present invention causes a greater decrease in cccDNA titre than the comprised therapeutic oligonucleotide or TLR7 agonist alone.

In a preferred embodiment of the present invention, the pharmaceutical combination comprises or consists of an RTEL1 targeting oligonucleotide and a FUBP1 targeting oligonucleotide, or a conjugate thereof.

In a preferred embodiment of the present invention, the pharmaceutical combination comprises or consists of an RTEL1 targeting single-stranded antisense oligonucleotide and a FUBP1 targeting single-stranded antisense oligonucleotide, or a conjugate thereof.

The RTEL1 targeting single-standard antisense oligonucleotide may be a RTEL1 targeting single-stranded antisense oligonucleotide as described herein. The FUBP1 targeting single-standard antisense oligonucleotide may be a FUBP1 targeting single-stranded antisense oligonucleotide as described herein.

### Applications

The pharmaceutical combination of the present invention is for use in treatment of Hepatitis B virus infections and/or cancer, in particular treatment of patients with chronic HBV.

The pharmaceutical combination of the invention may be utilized as research reagent or in diagnostics, therapeutics and in prophylaxis.

The pharmaceutical combination of the invention can be used as a combined hepatitis B virus targeting therapy and an immunotherapy.

In research, such combination may be used to specifically modulate the synthesis of RTEL1 protein in cells (e.g. in vitro cell cultures) and experimental animals thereby facilitating functional analysis of the target or an appraisal of its usefulness as a target for therapeutic intervention. Typically, the target modulation is achieved by degrading or inhibiting the mRNA producing the protein, thereby prevent protein formation or by degrading or inhibiting a modulator of the gene or mRNA producing the protein.

If employing the combination of the invention in research or diagnostics the target nucleic acid may be a cDNA or a synthetic nucleic acid derived from DNA or RNA.

Also encompassed by the present invention is an in vivo or in vitro method for modulating RTEL1 expression in a target cell, which is expressing RTEL1, said method comprising administering a combination of the invention in an effective amount to said cell.

In some embodiments, the target cell, is a mammalian cell in particular a human cell. The target cell may be an in vitro cell culture or an in vivo cell forming part of a tissue in a mammal. In preferred embodiments, the target cell is present in in the liver. The target cell may be a hepatocyte.

One aspect of the present invention is related the combination of the invention, for use as a medicament.

In an aspect of the invention, the combination of the invention is capable of reducing the cccDNA level in the infected cells and therefore inhibiting HBV infection. In particular, the combination is capable of affecting one or more of the following parameters i) reducing cccDNA and/or ii) reducing pgRNA and/or iii) reducing HBV DNA and/or iv) reducing HBV viral antigens in an infected cell.

For example, combinations that inhibits HBV infection may reduce i) the cccDNA levels in an infected cell by at least 40% such as 50%, 60%, 70%, 80%, or 90% reduction compared to controls; or ii) the level of pgRNA by at least 40% such as 50%, 60%, 70%, 80%, or 90% reduction compared to controls. The controls may be untreated cells or animals, or cells or animals treated with an appropriate control.

Inhibition of HBV infection may be measured in vitro using HBV infected primary human hepatocytes or in vivo using humanized hepatocytes PXB mouse model (available at PhoenixBio, see also Kakuni et al 2014 Int. J. Mol. Sci. 15:58-74). Inhibition of secretion of HBsAg and/or HBeAg may be measured by ELISA, e.g. by using the CLIA ELISA Kit (Autobio Diagnostic) according to the manufacturers' instructions. Reduction of intracellular cccDNA or HBV mRNA and pgRNA may be measured by qPCR, e.g. as described in the Materials and Methods section. Further methods for evaluating whether a test compound inhibits HBV infection are measuring secretion of HBV DNA by qPCR e.g. as described in WO 2015/173208 or using Northern Blot; in-situ hybridization, or immuno-fluorescence.

Due to the reduction of RTEL1 levels, the combination of the present invention can be used to inhibit development of or in the treatment of HBV infection. In particular, the destabilization and reduction of the cccDNA, the combination of the present invention more efficiently inhibits development of, or treats, a chronic HBV infection as compared to a compound that only reduces secretion of HBsAg.

Accordingly, one aspect of the present invention is related to use of the combination of the invention to reduce cccDNA and/or pgRNA in an HBV infected individual.

A further aspect of the invention relates to the use of the combination of the invention to inhibit development of or treat a chronic HBV infection.

A further aspect of the invention relates to the use of the combination of the invention to reduce the infectiousness of a HBV infected person. In a particular aspect of the invention, the combination of the invention inhibits development of a chronic HBV infection.

The subject to be treated with the combination of the invention (or which prophylactically receives the composition of the present invention) is preferably a human, more preferably a human patient who is HBsAg positive and/or HBeAg positive, even more preferably a human patient that is HBsAg positive and HBeAg positive.

Accordingly, the present invention relates to a method of treating a HBV infection, wherein the method comprises administering an effective amount of the combination of the invention. The present invention further relates to a method of preventing liver cirrhosis and hepatocellular carcinoma caused by a chronic HBV infection.

The invention also provides for the use of combination of the invention for the manufacture of a medicament, in particular a medicament for use in the treatment of HBV infection or chronic HBV infection or reduction of the infectiousness of a HBV infected person. In preferred embodiments, the medicament is manufactured in a dosage form for subcutaneous administration.

The invention also provides for the use of combination of the invention for the manufacture of a medicament wherein the medicament is in a dosage form for intravenous administration.

The combination of the invention may be used in a combination therapy. For example, the combination of the invention may be combined with other anti-HBV agents such as interferon alpha-2b, interferon alpha-2a, and interferon alphacon-1 (pegylated and unpegylated), ribavirin, lamivudine (3TC), entecavir, tenofovir, telbivudine (LdT), adefovir, or other emerging anti-HBV agents such as a HBV RNA replication inhibitor, a HBsAg secretion inhibitor, a HBV capsid inhibitor, an antisense oligomer (e.g. as described in WO2012/145697, WO 2014/179629 and WO2017/216390), a siRNA (e.g. described in WO 2005/014806, WO 2012/024170, WO 2012/2055362, WO 2013/003520, WO 2013/159109, WO 2017/027350 and WO2017/015175), a HBV therapeutic vaccine, a HBV prophylactic vaccine, a HBV antibody therapy (monoclonal or polyclonal), or TLR 2, 3, 7, 8 or 9 agonists for the treatment and/or prophylaxis of HBV.

### Embodiments of the Invention

The following embodiments of the present invention may be used in combination with any other embodiments described herein.

1. A composition comprising an inhibitor of RTEL1 and an inhibitor of FUBP1.

2. A pharmaceutical composition comprising an inhibitor of RTEL1 and an inhibitor of FUBP1, or a pharmaceutically acceptable salt thereof, and a pharmaceutically acceptable diluent, solvent, carrier, salt and/or adjuvant.

3. A kit comprising an inhibitor of RTEL1 and an inhibitor of FUBP1.

4. The composition according to item 1 or 2, or the kit according to item 3 wherein the inhibitor of RTEL1 is capable of reducing cccDNA in an infected cell.

5. The composition or the kit according to any of the preceding items, wherein the RTEL1 inhibitor is an nucleic acid molecule of 12 to 60 nucleotides in length, preferably 12 to 30 nucleotides in length, more preferably 12 to 25, even more preferably 15 to 21 nucleotides in length, comprising a contiguous nucleotide sequence of at least 10 nucleotides in length which is at least 80%, preferably at least 85%, more preferably at least 90%, even more preferably at least 95% complementary to a mammalian RTEL1 target nucleic acid, in particular a human RTEL1 target nucleic acid, wherein the nucleic acid molecule is capable of reducing the expression of RTEL1.

6. The composition or the kit according to item 5, wherein the mammalian RTEL1 target nucleic acid is selected from SEQ ID NO: 1 or 2.

7. The composition or the kit according to items 5 or 6, wherein the contiguous nucleotide sequence is at least 80%, preferably at least 85%, more preferably at least 90%, even more preferably at least 95% complementary to SEQ ID NO: 1 and/or 2, preferably SEQ ID NO: 1.

8. The composition or the kit according to any of items 5 to 7, wherein the contiguous nucleotide sequence is at least 98% complementarity to the target nucleic acid of SEQ ID NO: 1 and/or SEQ ID NO: 2, preferably SEQ ID NO: 1.

9. The composition or the kit according to any of items 5 to 8, wherein the contiguous nucleotide sequence is 100% complementarity to the target nucleic acid of SEQ ID NO: 1 and/or SEQ ID NO: 2, preferably SEQ ID NO: 1.

10. The composition or the kit according to any of items 5 to 9, wherein the contiguous nucleotide sequence is at least 80%, such as at least 85%, such as at least 90%, such as at least 95%, such as at least 98, such as 100% complementary to a target sequence selected from SEQ ID NO: 3-26, preferably 100% complementary to a target sequence selected from SEQ ID NO: 5, 13, 14, 15, 16; more preferably 100% complementary to a target sequence selected from SEQ ID NO: 14 and 16.

11. The composition or the kit according to any of the preceding items, wherein the RTEL1 inhibitor is selected from a single stranded antisense oligonucleotide, siRNA or a shRNA molecule.

12. The composition or the kit according to any of the preceding items, wherein the RTEL1 inhibitor is a single stranded antisense oligonucleotide.

13. The composition or the kit according to any of the preceding items, wherein the RTLE1 inhibitor is a single stranded antisense oligonucleotide of 12-30 nucleotides in length comprising a contiguous nucleotides sequence of at least 10 nucleotides which is complementary to a mammalian RTEL1 target nucleic acid, such as a RTEL1 pre-mRNA, such as a RTEL1 pre-mRNA of SEQ ID NO: 1 or 2, in particular a human RTEL1 target nucleic acid, such as a human RTEL1 pre-mRNA, such as a human RTEL1 pre-mRNA of SEQ ID NO: 1, wherein the oligonucleotide is capable of reducing the expression of RTEL1.

14. The composition or the kit according to item 13, wherein the contiguous nucleotide sequence is 10, 11, 12, 13, 14, 15, 16, 17, 18, 19, 20, 21, 22, 23, 24, 25, 26, 27, 28, 29 or 30 nucleotides in length.

15. The composition or the kit according to item 13 or 14, wherein the contiguous nucleotide sequence is of 12 to 25, in particular 15 to 21 nucleotides in length.

16. The composition or the kit according to item 12 to 15, wherein the antisense oligonucleotide comprises a sequence selected from the group consisting of SEQ ID NO: 27-246.

17. The composition or the kit according to any of items 12 to 16, wherein the antisense oligonucleotide comprises one or more 2′ sugar modified nucleoside.

18. The composition or the kit according to item 17, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides 19. The composition or the kit according to items 17 or 18, wherein the one or more 2′ sugar modified nucleoside is a LNA nucleoside.

20. The composition or the kit according to any of items 12 to 19, wherein the antisense oligonucleotide comprises at least one phosphorothioate internucleoside linkage.

21. The composition or the kit according to any of items 12 to 20, wherein all the internucleoside linkages within the contiguous nucleotide sequence are phosphorothioate internucleoside linkages.

22. The composition or the kit according to any of items 12 to 21, wherein the oligonucleotide is capable of recruiting RNase H.

23. The composition or the kit according to any of items 12 to 22, wherein the antisense oligonucleotide, or contiguous nucleotide sequence thereof, consists or comprises a gapmer of formula 5′-F-G-F′-3′, where region F and F′ independently comprise 1-4 2′ sugar modified nucleosides and G is a region between 6 and 16 nucleosides which are capable of recruiting RNaseH, such as a region comprising between 6 and 18 DNA nucleosides.

24. The composition or the kit according to any of items 12 to 23, wherein the antisense oligonucleotide capable of reducing the expression of RTEL1 is selected from the group of antisense oligonucleotides comprising or consisting of

(SEQ ID NO: 243)
AATTttacatactctgGT,
(SEQ ID NO: 244)
AAttttacatactctGGTC,
(SEQ ID NO: 245)
TTacatactctggtCAAA,
(SEQ ID NO: 246)
CTttattataactTgaAtCTC,
and
(SEQ ID NO: 246)
CTttattataacttgaaTCTC;
(SEQ ID NO: 245)
preferably TTacatactctggtCAAA;
or
(SEQ ID NO: 246)
CTttattataacttgaaTCTC;

wherein capital letters are beta-D-oxy LNA nucleosides, lowercase letters are DNA nucleosides, all LNA C are 5-methyl cytosine, and all internucleoside linkages are phosphorothioate internucleoside linkages.

25. The composition or the kit according to item 11, wherein the RTEL1 inhibitor is a shRNA.

26. The composition or the kit according to item 11, wherein the RTEL1 inhibitor is a siRNA.

27. The composition or the kit according to any of the preceding items, wherein the RTEL1 inhibitor is covalently attached to at least one conjugate moiety.

28. The composition or the kit according to item 27, wherein the conjugate moiety comprises at least one asialoglycoprotein receptor targeting moiety selected from the group consisting of galactose, galactosamine, N-formyl-galactosamine, N-acetylgalactosamine, N-propionyl-galactosamine, N-n-butanoyl-galactosamine and N-isobutanoylgalactosamine.

29. The composition or the kit according to item 28, wherein the asialoglycoprotein receptor targeting moiety is N-acetylgalactosamine (GalNAc).

30. The composition or the kit according to item 27 or 28, wherein the conjugate moiety is mono-valent, di-valent, tri-valent or tetra-valent with respect to asialoglycoprotein receptor targeting moieties.

31. The composition or the kit according to item 30, wherein the conjugate moiety consists of two to four terminal GalNAc moieties and a spacer linking each GalNAc moiety to a brancher molecule that can be conjugated to the antisense compound.

32. The composition or the kit according to item 31, wherein the spacer is a PEG spacer.

33. The composition or the kit according to any one of item 28 to 32, wherein the conjugate moiety is a tri-valent N-acetylgalactosamine (GalNAc) moiety.

34. The composition or the kit according to any one of item 28 to 33, wherein the conjugate moiety is selected from one of the trivalent GalNAc moieties in FIG. 5.

35. The composition or the kit according to any item 34, wherein the conjugate moiety is the trivalent GalNAc moiety in FIG. 5, such as the trivalent GalNAc moiety of FIG. 5D-1 or FIG. 5D-2, or a mixture of both.

36. The composition or the kit according to any one of item 28 to 35, comprising a linker, which is positioned between the antisense oligonucleotide and the conjugate moiety, preferably wherein the linker is a CA DNA dinucleotide.

37. The composition or the kit according to any one of item 28 to 36, wherein the conjugate is selected from the group consisting of

5′-GN2-C6ₒ[X]AₛAₛTₛTₛtₛtₛaₛcₛaₛtₛaₛcₛtₛcₛtₛgₛGₛT,
5′-GN2-C6ₒ[X]AₛAₛtₛtₛtₛtₛaₛcₛaₛtₛaₛcₛtₛcₛtₛGₛGₛTₛ^{m}C,
5′-GN2-C6ₒ[X]TₛTₛaₛcₛaₛtₛaₛcₛtₛcₛtₛgₛgₛtₛ^{m}CₛAₛAₛA,
5′-GN2-C6ₒ[X]^{m}CₛTₛtₛtₛaₛtₛtₛaₛtₛaₛaₛcₛtₛTₛgₛaₛAₛtₛ^{m}CₛTₛ^{m}C,
and
5′-GN2-C6ₒ[X]^{m}CₛTₛtₛtₛaₛtₛtₛaₛtₛaₛaₛcₛtₛtₛgₛaₛaₛTₛ^{m}CₛTₛ^{m}C;
preferably 5′-GN2-C6ₒ[X]TₛTₛaₛcₛaₛtₛaₛcₛtₛcₛtₛgₛgₛtₛ
^{m}CₛAₛAₛA,
or
5′-GN2-C6ₒ[X]^{m}CₛTₛtₛtₛaₛtₛtₛaₛtₛaₛaₛcₛtₛtₛgₛaₛaₛTₛ^{m}CₛTₛ^{m}C;

wherein a capital letter represents a beta-D-oxy LNA nucleoside, a lower case letter represents a DNA nucleoside, wherein each LNA cytosine is 5-methyl cytosine, and mc is 5-methyl cytosine DNA, and wherein subscript s represents a phosphorothioate internucleoside linkage, and a subscript o represents a phosphodiester internucleoside linkage, and GN2-C6 is a residue of formula:

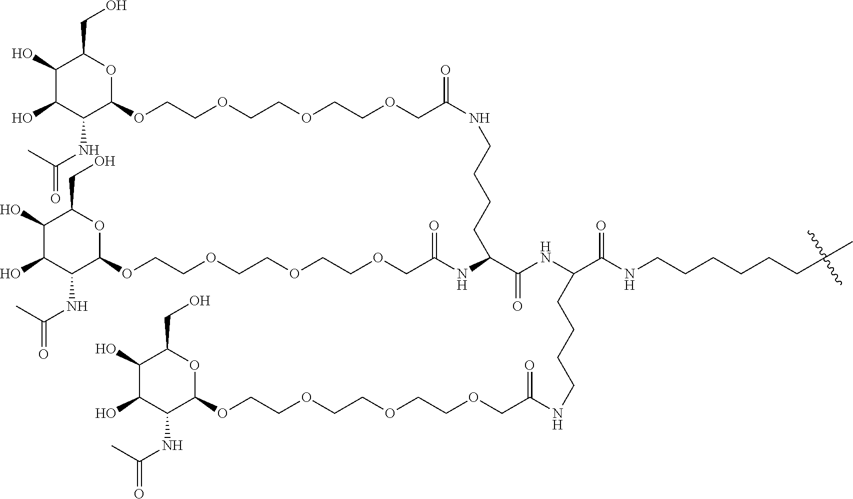 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

wherein the residue GN2-C6 is attached via a phosphodiester linkage at the 5′ end of the oligonucleotide, and/or wherein GN2-C6 is a tri-valent N-acetylgalactosamine (GalNAc) of FIG. 5D1 or FIG. 5D2, or a mixture of both, more preferably wherein GN2-C6 is a mixture of the tri-valent N-acetylgalactosamine (GalNAc) residues depicted in FIG. 5D1 or FIG. 5D2, and wherein and [X] represents cₒaₒ in accordance with the foregoing.

38. The composition or the kit according to any one of item 28 to 37, wherein the conjugate is the conjugate as shown in FIG. 1.

39. The composition or the kit according to any one of item 28 to 37, wherein the conjugate is the conjugate as shown in FIG. 2.

40. The composition or the kit according to any one of item 28 to 37, wherein the conjugate is the conjugate as shown in FIG. 3.

41. The composition or the kit according to any one of item 28 to 37, wherein the conjugate is the conjugate as shown in FIG. 4.

42. The composition or the kit according to any of the preceding items, wherein the RTEL1 inhibitor is in the form of a pharmaceutically acceptable salt.

43. The composition or the kit according to item 28, wherein the salt is the salt is a sodium salt, a potassium salt or an ammonium salt.

44. The composition or the kit according to any of the preceding items, wherein the composition comprises an aqueous diluent or solvent, such as phosphate buffered saline.

45. The composition or the kit according to any of the preceding items, wherein the inhibitor of FUBP1 is capable of reducing cccDNA and/or pgRNA in an infected cell.

46. The composition or the kit according to any of the preceding items, wherein the FUBP1 inhibitor is a nucleic acid molecule of 12 to 60 nucleotides in length in length, preferably 12 to 30 nucleotides in length, more preferably 12 to 25, even more preferably 15 to 21 nucleotides in length, which comprises or consists of a contiguous nucleotide sequence of 10 to 30 nucleotides in length, preferably 12 to 25, in particular 15 to 21 nucleotides in length, wherein the contiguous nucleotide sequence is at least 80%, preferably at least 85%, more preferably at least 90%, even more preferably at least 95% complementarity to a mammalian FUBP1 target nucleic acid, in particular a human FUBP1 target nucleic acid, wherein the nucleic acid molecule is capable of inhibiting the expression of FUBP1.

47. The composition or the kit according to item 46, wherein the mammalian FUBP1 target nucleic acid is selected from SEQ ID NO: 247 to 254.

48. The composition or the kit according to item 46 or 47, wherein the contiguous nucleotide sequence is at least 80%, preferably at least 85%, more preferably at least 90%, even more preferably at least 95% complementary to SEQ ID NO: 247 and/or 251, preferably SEQ ID NO: 247.

49. The composition or the kit according to any of items 46 to 48, wherein the contiguous nucleotide sequence is at least 98% complementarity to the target nucleic acid of SEQ ID NO: 247 and/or SEQ ID NO: 251, preferably SEQ ID NO: 247.

50. The composition or the kit according to any of items 46 to 49, wherein the contiguous nucleotide sequence is 100% complementarity to the target nucleic acid of SEQ ID NO: 247 and/or SEQ ID NO: 251, preferably SEQ ID NO: 247.

51. The composition or the kit according to any of items 46 to 50, wherein the contiguous nucleotide sequence is at least 90% complementary to a region within exon 14 or exon 20 of human FUBP1 (see Table 4).

52. The composition or the kit according to any of items 46 to 51, wherein the contiguous nucleotide sequence is 100% complementary to a region within exon 14 or exon 20 of human FUBP1 (see Table 4).

53. The composition or the kit according to any of items 46 to 50, wherein the contiguous nucleotide sequence is at least 80%, preferably at least 85%, more preferably at least 90%, even more preferably at least 95% complementary to a target sequence selected from the group consisting of position 9141-9156, 16184 to 16205, 16188 to 16205, 16184 to 16203, 16184 to 16200, 16186 to 16203, 16189 to 16205 or 30536 to 30553 of SEQ ID NO: 247.

54. The composition or the kit according to any of items 46 to 53, wherein the contiguous nucleotide sequence is 100% complementary to a target sequence selected from the group consisting of position 9141-9156, 16184 to 16205, 16188 to 16205, 16184 to 16203, 16184 to 16200, 16186 to 16203, 16189 to 16205 or 30536 to 30553 of SEQ ID NO: 247.

55. The composition or the kit according to any of the preceding items, wherein the FUBP1 inhibitor is selected from a single stranded antisense oligonucleotide, siRNA or a shRNA molecule.

56. The composition or the kit according to any of the preceding items, wherein the FUBP1 inhibitor is a single stranded antisense oligonucleotide.

57. The composition or the kit according to any of the preceding items, wherein the FUBP1 inhibitor is a single stranded antisense oligonucleotide of 12-30 nucleotides in length comprising a contiguous nucleotides sequence of at least 10 nucleotides which is complementary to a mammalian FUBP1, in particular a human FUBP1, wherein the oligonucleotide is capable of inhibiting the expression of FUBP1.

58. The composition or the kit according to item 57, wherein the contiguous nucleotide sequence is 10, 11, 12, 13, 14, 15, 16, 17, 18, 19, 20, 21, 22, 23, 24, 25, 26, 27, 28, 29 or 30 nucleotides in length.

59. The composition or the kit according to item 57 or 58, wherein the contiguous nucleotide sequence is of 12 to 25, in particular 15 to 21 nucleotides in length.

60. The composition or the kit according to item 56 to 59, wherein the single stranded antisense oligonucleotide comprises or consists of a sequence selected from the group consisting of SEQ ID NO: 275-330.

61. The composition or the kit according to any of items 56 to 60, wherein the antisense oligonucleotide comprises one or more 2′ sugar modified nucleoside.

62. The composition or the kit according to item 61, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides 63. The composition or the kit according to items 61 or 62, wherein the one or more 2′ sugar modified nucleoside is a LNA nucleoside.

64. The composition or the kit according to any of items 56 to 63, wherein the antisense oligonucleotide comprises at least one phosphorothioate internucleoside linkage.

65. The composition or the kit according to any of items 56 to 64, wherein all the internucleoside linkages within the contiguous nucleotide sequence are phosphorothioate internucleoside linkages.

66. The composition or the kit according to any of items 56 to 65, wherein the oligonucleotide is capable of recruiting RNase H.

67. The composition or the kit according to any of items 56 to 66, wherein the antisense oligonucleotide, or contiguous nucleotide sequence thereof, consists or comprises a gapmer of formula 5′-F-G-F′-3′, where region F and F′ independently comprise 1-4 2′ sugar modified nucleosides and G is a region between 6 and 16 nucleosides which are capable of recruiting RNaseH, such as a region comprising between 6 and 18 DNA nucleosides.

68. The composition or the kit according to item 56 to 67, wherein the single stranded antisense oligonucleotide capable of inhibiting the expression of FUBP1 is selected from the group of antisense oligonucleotides comprising or consisting of

(SEQ ID NO: 325)
CTTatGctttttatgGT,
(SEQ ID NO: 325)
CTTaTgctttttatgGT,
(SEQ ID NO: 326)
CTtATgctttttatgGTT,
(SEQ ID NO: 326)
CTtAtgctttttatgGTT,
(SEQ ID NO: 326)
CTtAtgctttttatGgTT,
(SEQ ID NO: 326)
CTtAtgctttttatGGTT,
(SEQ ID NO: 327)
GcttTttatggtTtCAC,
(SEQ ID NO: 328)
TATgcTttttatggtTTC,
(SEQ ID NO: 329)
AcCAAttttcatttCtAC,
and
(SEQ ID NO: 330)
CcccataaccataGTC;
(SEQ ID NO: 326)
preferably CTtAtgctttttatGgTT;
or
(SEQ ID NO: 330)
CcccataaccataGTC;

wherein capital letters are beta-D-oxy LNA nucleosides, lowercase letters are DNA nucleosides, all LNA C are 5-methyl cytosine, and all internucleoside linkages are phosphorothioate internucleoside linkages.

69. The composition or the kit according to item 55, wherein the FUBP1 inhibitor is a shRNA.

70. The composition or the kit according to item 55, wherein the FUBP1 inhibitor is a siRNA.

71. The composition or the kit according to any of the preceding items, wherein the FUBP1 inhibitor is covalently attached to at least one conjugate moiety.

72. The composition or the kit according to item 71, wherein the conjugate moiety comprises at least one asialoglycoprotein receptor targeting moiety selected from the group consisting of galactose, galactosamine, N-formyl-galactosamine, N-acetylgalactosamine, N-propionyl-galactosamine, N-n-butanoyl-galactosamine and N-isobutanoylgalactosamine.

73. The composition or the kit according to item 72, wherein the asialoglycoprotein receptor targeting moiety is N-acetylgalactosamine (GalNAc).

74. The composition or the kit according to item 71 or 72, wherein the conjugate moiety is mono-valent, di-valent, tri-valent or tetra-valent with respect to asialoglycoprotein receptor targeting moieties.

75. The composition or the kit according to item 74, wherein the conjugate moiety consists of two to four terminal GalNAc moieties and a spacer linking each GalNAc moiety to a brancher molecule that can be conjugated to the antisense compound.

76. The composition or the kit according to item 75, wherein the spacer is a PEG spacer.

77. The composition or the kit according to any one of item 71 to 76, wherein the conjugate moiety is a tri-valent N-acetylgalactosamine (GalNAc) moiety.

78. The composition or the kit according to any one of item 71 to 77, wherein the conjugate moiety is selected from one of the trivalent GalNAc moieties in FIG. 5.

79. The composition or the kit according to any item 71, wherein the conjugate moiety is the trivalent GalNAc moiety in FIG. 5, such as the trivalent GalNAc moiety of FIG. 5D-1 or FIG. 5D-2, or a mixture of both.

80. The composition or the kit according to any one of item 71 to 79, comprising a linker, which is positioned between the antisense oligonucleotide and the conjugate moiety, preferably wherein the linker is a CA DNA dinucleotide.

81. The composition or the kit according to any one of item 71 to 80, wherein the conjugate is selected from the group consisting of

5′-GN2-C6ₒ[X]^{m}CₛTₛTₛaₛtₛGₛcₛtₛtₛtₛtₛtₛaₛtₛgₛGₛT,
5′-GN2-C6ₒ[X]^{m}CₛTₛTₛaₛTₛgₛcₛtₛtₛtₛtₛtₛaₛtₛgₛGₛT,
5′-GN2-C6ₒ[X]^{m}CₛTₛtₛAₛTₛgₛcₛtₛtₛtₛtₛtₛaₛtₛgₛGₛTₛT,
5′-GN2-C6ₒ[X]^{m}CₛTₛtₛAₛtₛgₛcₛtₛtₛtₛtₛtₛaₛtₛgₛGₛTₛT,
5′-GN2-C6ₒ[X]^{m}CₛTₛtₛAₛtₛgₛcₛtₛtₛtₛtₛtₛaₛtₛGₛgₛTₛT,
5′-GN2-C6ₒ[X]^{m}CₛTₛtₛAₛtₛgₛcₛtₛtₛtₛtₛtₛaₛtₛGₛGₛTₛT,
5′-GN2-C6ₒ[X]GₛcₛtₛtₛTₛtₛtₛaₛtₛgₛgₛtₛTₛtₛ^{m}CₛAₛ^{m}C,
5′-GN2-C6ₒ[X]TₛAₛTₛgₛcₛTₛtₛtₛtₛtₛaₛtₛgₛgₛtₛTₛTₛ^{m}C,
5′-GN2-C6ₒ[X]AₛCₛ^{m}CₛAₛAₛtₛtₛtₛtₛcₛaₛtₛtₛtₛ^{m}CₛtAₛ^{m}C,
and
5′-GN2-C6ₒ[X]^{m}CₛcₛcₛcₛaₛtₛaₛaₛcₛcₛaₛtₛaₛGₛTₛ^{m}Cₛ;
preferably 5′-GN2-C6ₒ[X]^{m}CₛTₛtₛAₛtₛgₛcₛtₛtₛtₛtₛtₛaₛtₛ
GₛgₛTₛ;
or
5′-GN2-C6ₒ[X]^{m}CₛcₛcₛcₛaₛtₛaₛaₛcₛcₛaₛtₛaₛGₛTₛ^{m}Cₛ;

wherein a capital letter represents a beta-D-oxy LNA nucleoside, a lower case letter represents a DNA nucleoside, wherein each LNA cytosine is 5-methyl cytosine, and mC is 5-methyl cytosine DNA, and wherein subscript s represents a phosphorothioate internucleoside linkage, and a subscript o represents a phosphodiester internucleoside linkage, and GN2-C6 is a residue of formula:

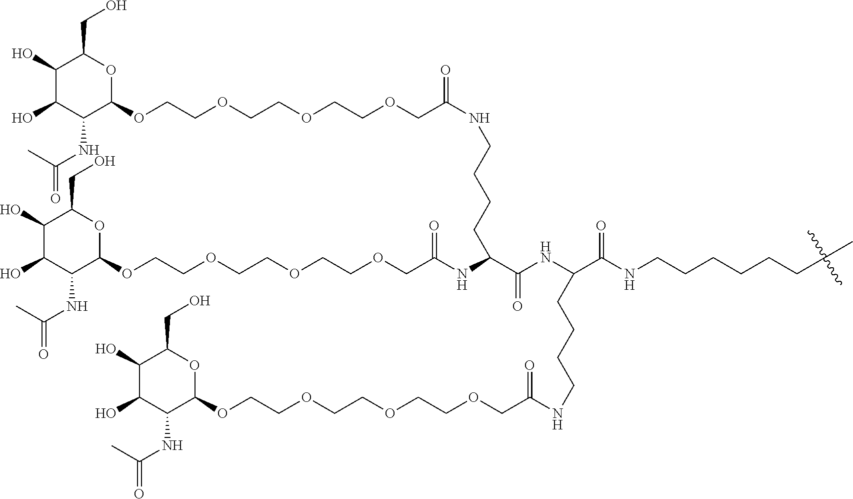 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

wherein the residue GN2-C6 is attached via a phosphodiester linkage at the 5′ end of the oligonucleotide, and/or wherein GN2-C6 is a tri-valent N-acetylgalactosamine (GalNAc) of FIG. 5D1 or FIG. 5D2, or a mixture of both, more preferably wherein GN2-C6 is a mixture of the tri-valent N-acetylgalactosamine (GalNAc) residues depicted in FIG. 5D1 or FIG. 5D2, and wherein and [X] represents cₒaₒ in accordance with the foregoing.

82. The composition or the kit according to any one of item 71 to 81, wherein the conjugate is the conjugate as shown in FIG. 8.

83. The composition or the kit according to any one of item 71 to 81, wherein the conjugate is the conjugate as shown in FIG. 9.

84. The composition or the kit according to any one of item 71 to 81, wherein the conjugate is the conjugate as shown in FIG. 10.

85. The composition or the kit according to any one of item 71 to 81, wherein the conjugate is the conjugate as shown in FIG. 11.

86. The composition or the kit according to any one of item 71 to 81, wherein the conjugate is the conjugate as shown in FIG. 12.

87. The composition or the kit according to any one of item 71 to 81, wherein the conjugate is the conjugate as shown in FIG. 13.

88. The composition or the kit according to any one of item 71 to 81, wherein the conjugate is the conjugate as shown in FIG. 14.

89. The composition or the kit according to any one of item 71 to 81, wherein the conjugate is the conjugate as shown in FIG. 15.

90. The composition or the kit according to any one of item 71 to 81, wherein the conjugate is the conjugate as shown in FIG. 16.

91. The composition or the kit according to any of the preceding items, wherein the FUBP1 inhibitor is in the form of a pharmaceutically acceptable salt.

92. The composition or the kit according to item 91, wherein the salt is the salt is a sodium salt, a potassium salt or an ammonium salt.

93. The composition or the kit according to any of the preceding items, wherein the composition comprises an aqueous diluent or solvent, such as phosphate buffered saline 94. The composition or the kit according to any of claims 1 to 44, wherein the FUBP1 inhibitor is selected from the compounds of Formula VII, IX or X

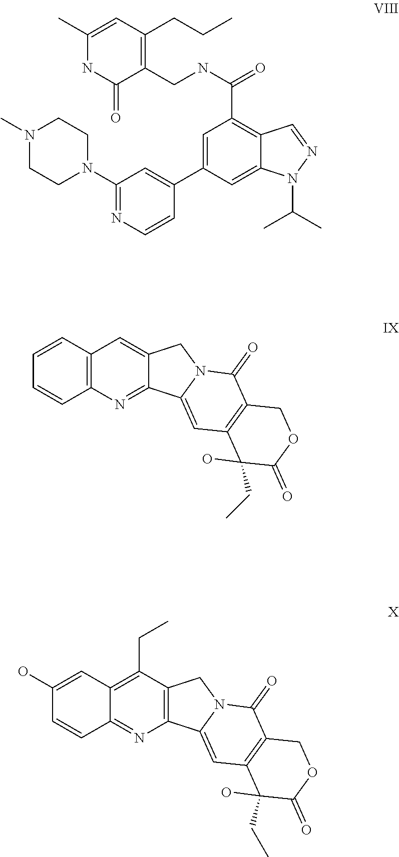 [SMILES: CCCc1cc(C)[nH]c(=O)c1CNC(=O)c1cc(-c2ccnc(N3CCN(C)CC3)c2)cc2c1cnn2C(C)C ; CC[C@@]1(O)C(=O)OCc2c1cc1n(c2=O)Cc2cc3ccccc3nc2-1 ; CCc1c2c(nc3ccc(O)cc13)-c1cc3c(c(=O)n1C2)COC(=O)[C@]3(O)CC]

95. The composition or the kit according to any of the preceding items, wherein the inhibitor of RTEL1 is a single stranded antisense oligonucleotide capable of inhibiting the expression of RTEL1, comprising or consisting of AATTttacatactctgGT (SEQ ID NO: 243), and wherein the inhibitor of FUBP1 is a single stranded antisense oligonucleotide capable of reducing the expression of FUBP1, which is selected from the group of antisense oligonucleotides comprising or consisting of:
CTTatGctttttatgGT (SEQ ID NO: 325),
CTTaTgctttttatgGT (SEQ ID NO: 325),
CTtATgctttttatgGTT (SEQ ID NO: 326),
CTtAtgctttttatgGTT (SEQ ID NO: 326),
CTtAtgctttttatGgTT (SEQ ID NO: 326),
CTtAtgctttttatGGTT (SEQ ID NO: 326),
GcttTttatggtTtCAC (SEQ ID NO: 327),
TATgcTttttatggtTTC (SEQ ID NO: 328),
AcCAAttttcatttCtAC (SEQ ID NO: 329), and
CcccataaccataGTC (SEQ ID NO: 330);
   wherein capital letters are beta-D-oxy LNA nucleosides, lowercase letters are DNA nucleosides, all LNA C are 5-methyl cytosine, and all internucleoside linkages are phosphorothioate internucleoside linkages.

96. The composition or the kit according to item 95, wherein the inhibitor of RTEL1 is the conjugate consisting of 5′-GN2-C6ₒ[X]AₛAₛTₛTₛtₛtₛaₛcₛaₛtₛaₛcₛtₛcₛtₛgₛGₛT, such as shown in FIG. 1, and
wherein the inhibitor of FUBP1 is a conjugate selected from the group consisting of

5′-GN2-C6ₒ[X]^{m}CₛTₛTₛaₛtₛGₛcₛtₛtₛtₛtₛtₛaₛtₛgₛGₛT,
as shown in FIG. 8,
5′-GN2-C6ₒ[X]^{m}CₛTₛTₛaₛTₛgₛcₛtₛtₛtₛtₛtₛaₛtₛgₛGₛT,
as shown in FIG. 9,
5′-GN2-C6ₒ[X]^{m}CₛTₛtₛAₛTₛgₛcₛtₛtₛtₛtₛtₛaₛtₛgₛGₛTₛT,
as shown in FIG. 10,
5′-GN2-C6ₒ[X]^{m}CₛTₛtₛAₛtₛgₛcₛtₛtₛtₛtₛtₛaₛtₛgₛGₛTₛT,
as shown in FIG. 11,
5′-GN2-C6ₒ[X]^{m}CₛTₛtₛAₛtₛgₛcₛtₛtₛtₛtₛtₛaₛtₛGₛgₛTₛT,
as shown in FIG. 12,
5′-GN2-C6ₒ[X]^{m}CₛTₛtₛAₛtₛgₛcₛtₛtₛtₛtₛtₛaₛtₛGₛGₛTₛT,
as shown in FIG. 13,
5′-GN2-C6ₒ[X]GₛcₛtₛtₛTₛtₛtₛaₛtₛgₛgₛtₛTₛtₛ^{m}CₛAₛ^{m}C,
as shown in FIG. 14,
5′-GN2-C6ₒ[X]TₛAₛTₛgₛcₛTₛtₛtₛtₛtₛaₛtₛgₛgₛtₛTₛTₛ^{m}C,
as shown in FIG. 15,
5′-GN2-C6ₒ[X]Aₛcₛ^{m}CₛAₛAₛtₛtₛtₛtₛcₛaₛtₛtₛtₛ^{m}CₛtAₛ^{m}C,
as shown in FIG. 16,
and
5′-GN2-C6ₒ[X]^{m}CₛcₛcₛcₛaₛtₛaₛaₛcₛcₛaₛtₛaₛGₛTₛ^{m}Cₛ;

wherein a capital letter represents a beta-D-oxy LNA nucleoside, a lower case letter represents a DNA nucleoside, wherein each LNA cytosine is 5-methyl cytosine, and ^{m}C is 5-methyl cytosine DNA, and wherein subscript s represents a phosphorothioate internucleoside linkage, and a subscript o represents a phosphodiester internucleoside linkage, and GN2-C6 is a residue of formula:

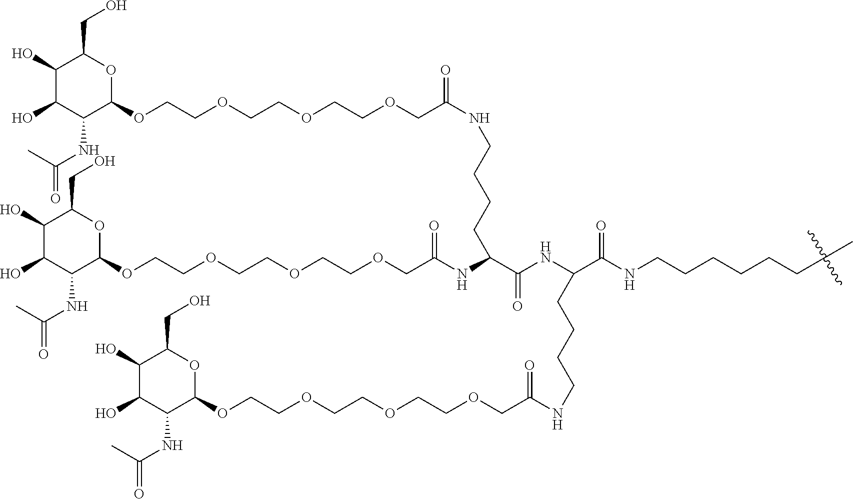 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

wherein the residue GN2-C6 is attached via a phosphodiester linkage at the 5′ end of the oligonucleotide, and/or wherein GN2-C6 is a tri-valent N-acetylgalactosamine (GalNAc) of FIG. 5D1 or FIG. 5D2, or a mixture of both, more preferably wherein GN2-C6 is a mixture of the tri-valent N-acetylgalactosamine (GalNAc) residues depicted in FIG. 5D1 or FIG. 5D2, and wherein and [X] represents cₒaₒ in accordance with the foregoing.

97. The composition or the kit according to any of the preceding items, wherein the inhibitor of RTEL1 is a single stranded antisense oligonucleotide capable of inhibiting the expression of RTEL1, comprising or consisting of AAttttacatactctGGTC (SEQ ID NO: 244), and wherein the inhibitor of FUBP1 is a single stranded antisense oligonucleotide capable of reducing the expression of FUBP1, which is selected from the group of antisense oligonucleotides comprising or consisting of:

(SEQ ID NO: 325)
CTTatGctttttatgGT,
(SEQ ID NO: 325)
CTTaTgctttttatgGT,
(SEQ ID NO: 326)
CTtATgctttttatgGTT,
(SEQ ID NO: 326)
CTtAtgctttttatgGTT,
(SEQ ID NO: 326)
CTtAtgctttttatGgTT,
(SEQ ID NO: 326)
CTtAtgctttttatGGTT,
(SEQ ID NO: 327)
GcttTttatggtTtCAC,
(SEQ ID NO: 328)
TATgcTttttatggtTTC,
(SEQ ID NO: 329)
AcCAAttttcatttCtAC

and
CcccataaccataGTC (SEQ ID NO: 330); wherein capital letters are beta-D-oxy LNA nucleosides, lowercase letters are DNA nucleosides, all LNA C are 5-methyl cytosine, and all internucleoside linkages are phosphorothioate internucleoside linkages.

98. The composition or the kit according to item 97, wherein the inhibitor of RTEL1 is the conjugate consisting of 5′-GN2-C6ₒ[X]AₛAₛtₛtₛtₛtₛaₛcₛaₛtₛaₛcₛtₛcₛtₛGₛGₛTₛ^{m}Cₛ, such as shown in FIG. 2, and
wherein the inhibitor of FUBP1 is a conjugate selected from the group consisting of

5′-GN2-C6ₒ[X]^{m}CₛTₛTₛaₛtₛGₛcₛtₛtₛtₛtₛtₛaₛtₛgₛGₛT,
as shown in FIG. 8,
5′-GN2-C6ₒ[X]^{m}CₛTₛTₛaₛTₛgₛcₛtₛtₛtₛtₛtₛaₛtₛgₛGₛT,
as shown in FIG. 9,
5′-GN2-C6ₒ[X]^{m}CₛTₛtₛAₛTₛgₛcₛtₛtₛtₛtₛtₛaₛtₛgₛGₛTₛT,
as shown in FIG. 10,
5′-GN2-C6ₒ[X]^{m}CₛTₛtₛAₛtₛgₛcₛtₛtₛtₛtₛtₛaₛtₛgₛGₛTₛT,
as shown in FIG. 11,
5′-GN2-C6ₒ[X]^{m}CₛTₛtₛAₛtₛgₛcₛtₛtₛtₛtₛtₛaₛtₛGₛgₛTₛT,
as shown in FIG. 12,
5′-GN2-C6ₒ[X]^{m}CₛTₛtₛAₛtₛgₛcₛtₛtₛtₛtₛtₛaₛtₛGₛGₛTₛT,
as shown in FIG. 13,
5′-GN2-C6ₒ[X]GₛcₛtₛtₛTₛtₛtₛaₛtₛgₛgₛtₛTₛtₛ^{m}CₛAₛ^{m}C,
as shown in FIG. 14,
5′-GN2-C6ₒ[X]TₛAₛTₛgₛcₛTₛtₛtₛtₛtₛaₛtₛgₛgₛtₛTₛTₛ^{m}C,
as shown in FIG. 15,
5′-GN2-C6ₒ[X]AₛCₛ^{m}CₛAₛAₛtₛtₛtₛtₛcₛaₛtₛtₛtₛ^{m}CₛtAₛ^{m}C,
as shown in FIG. 16,
and
5′GN2-C6ₒ[X]^{m}CₛcₛcₛcₛaₛtₛaₛaₛcₛcₛaₛtₛaₛGₛTₛ^{m}Cₛ;

wherein a capital letter represents a beta-D-oxy LNA nucleoside, a lower case letter represents a DNA nucleoside, wherein each LNA cytosine is 5-methyl cytosine, and ^{m}C is 5-methyl cytosine DNA, and wherein subscript s represents a phosphorothioate internucleoside linkage, and a subscript o represents a phosphodiester internucleoside linkage, and GN2-C6 is a residue of formula:

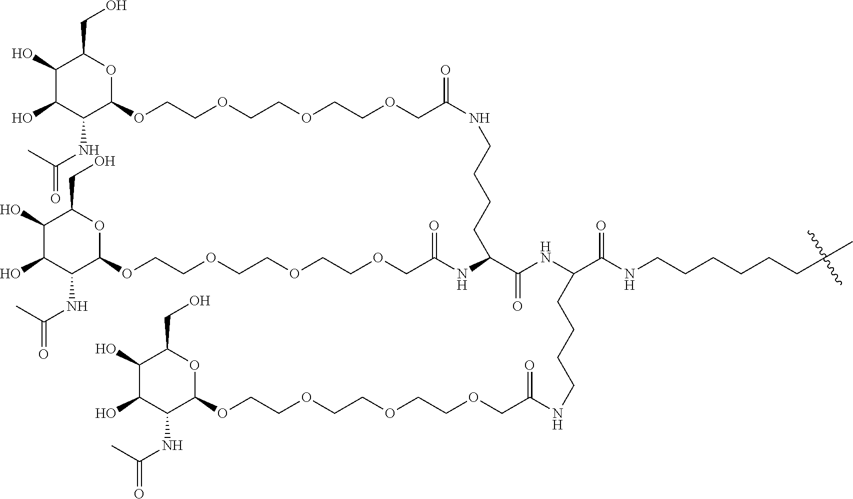 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

wherein the residue GN2-C6 is attached via a phosphodiester linkage at the 5′ end of the oligonucleotide, and/or wherein GN2-C6 is a tri-valent N-acetylgalactosamine (GalNAc) of FIG. 5D1 or FIG. 5D2, or a mixture of both, more preferably wherein GN2-C6 is a mixture of the tri-valent N-acetylgalactosamine (GalNAc) residues depicted in FIG. 5D1 or FIG. 5D2.

99. The composition or the kit according to any of the preceding items, wherein the inhibitor of RTEL1 is a single stranded antisense oligonucleotide capable of inhibiting the expression of RTEL1, comprising or consisting of TTacatactctggtCAAA (SEQ ID NO: 245), and wherein the inhibitor of FUBP1 is a single stranded antisense oligonucleotide capable of inhibiting the expression of FUBP1, which is selected from the group of antisense oligonucleotides comprising or consisting of:

(SEQ ID NO: 325)
CTTatGctttttatgGT,
(SEQ ID NO: 325)
CTTaTgctttttatgGT,
(SEQ ID NO: 326)
CTtATgctttttatgGTT,
(SEQ ID NO: 326)
CTtAtgctttttatgGTT,
(SEQ ID NO: 326)
CTtAtgctttttatGgTT,
(SEQ ID NO: 326)
CTtAtgctttttatGGTT,
(SEQ ID NO: 327)
GcttTttatggtTtCAC,
(SEQ ID NO: 328)
TATgcTttttatggtTTC,
(SEQ ID NO: 329)
AcCAAttttcatttCtAC
and
(SEQ ID NO: 330)
CcccataaccataGTC;
(SEQ ID NO: 326)
preferably CTtAtgctttttatGgTT;

wherein capital letters are beta-D-oxy LNA nucleosides, lowercase letters are DNA nucleosides, all LNA C are 5-methyl cytosine, and all internucleoside linkages are phosphorothioate internucleoside linkages.

100. The composition or the kit according to item 99, wherein the inhibitor of RTEL1 is the conjugate consisting of 5′-GN2-C6ₒ[X]TₛTₛaₛcₛaₛtₛaₛcₛtₛcₛtₛgₛgₛtₛ^{m}CₛAₛAₛAₛ, such as shown in FIG. 3, and wherein the inhibitor of FUBP1 is a conjugate selected from the group consisting of

5′-GN2-C6ₒ[X]^{m}CₛTₛTₛaₛtₛGₛcₛtₛtₛtₛtₛtₛaₛtₛgₛGₛT,
as shown in FIG. 8,
5′-GN2-C6ₒ[X]^{m}CₛTₛTₛaₛTₛgₛcₛtₛtₛtₛtₛtₛaₛtₛgₛGₛT,
as shown in FIG. 9,
5′-GN2-C6ₒ[X]^{m}CₛTₛtₛAₛTₛgₛcₛtₛtₛtₛtₛtₛaₛtₛgₛGₛTₛT,
as shown in FIG. 10,
5′-GN2-C6ₒ[X]^{m}CₛTₛtₛAₛtₛgₛcₛtₛtₛtₛtₛtₛaₛtₛgₛGₛTₛT,
as shown in FIG. 11,
5′-GN2-C6ₒ[X]^{m}CₛTₛtₛAₛtₛgₛcₛtₛtₛtₛtₛtₛaₛtₛGₛgₛTₛT,
as shown in FIG. 12,
5′-GN2-C6ₒ[X]^{m}CₛTₛtₛAₛtₛgₛcₛtₛtₛtₛtₛtₛaₛtₛGₛGₛTₛT,
as shown in FIG. 13,
5′-GN2-C6ₒ[X]GₛcₛtₛtₛTₛtₛtₛaₛtₛgₛgₛtₛTₛtₛ^{m}CₛAₛ^{m}C,
as shown in FIG. 14,
5′-GN2-C6ₒ[X]TₛAₛTₛgₛcₛTₛtₛtₛtₛtₛaₛtₛgₛgₛtₛTₛTₛ^{m}C,
as shown in FIG. 15,
5′GN2-C6ₒ[X]Aₛcₛ^{m}CₛAₛAₛtₛtₛtₛtₛcₛaₛtₛtₛtₛ^{m}CₛtAₛ^{m}C,
as shown in FIG. 16,
and
5′-GN2-C6ₒ[X]^{m}CₛcₛcₛcₛaₛtₛaₛaₛcₛcₛaₛtₛaₛGₛTₛ^{m}Cₛ;
preferably 5′-GN2-C6ₒ[X]^{m}CₛTₛtₛAₛtₛgₛcₛtₛtₛtₛtₛtₛaₛtₛ
GₛgₛTₛT,
as shown in FIG. 12;

wherein a capital letter represents a beta-D-oxy LNA nucleoside, a lower case letter represents a DNA nucleoside, wherein each LNA cytosine is 5-methyl cytosine, and ^{m}C is 5-methyl cytosine DNA, and wherein subscript s represents a phosphorothioate internucleoside linkage, and a subscript o represents a phosphodiester internucleoside linkage, and GN2-C6 is a residue of formula:

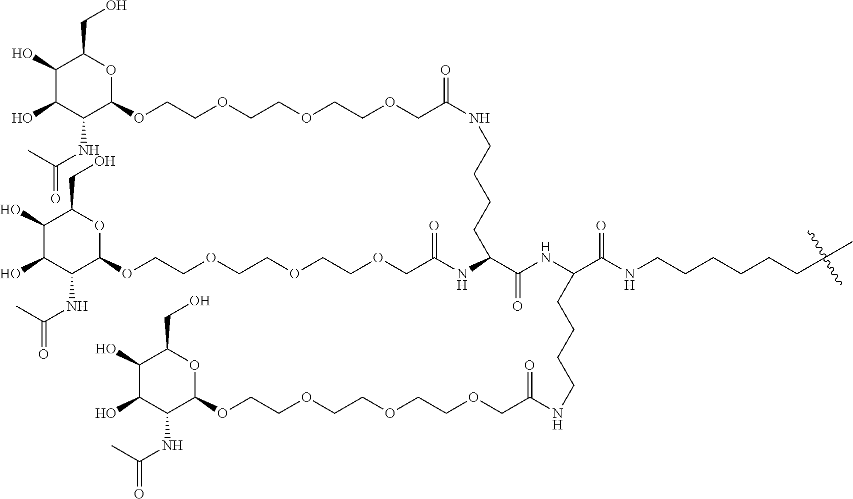 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

wherein the residue GN2-C6 is attached via a phosphodiester linkage at the 5′ end of the oligonucleotide, and/or wherein GN2-C6 is a tri-valent N-acetylgalactosamine (GalNAc) of FIG. 5D1 or FIG. 5D2, or a mixture of both, more preferably wherein GN2-C6 is a mixture of the tri-valent N-acetylgalactosamine (GalNAc) residues depicted in FIG. 5D1 or FIG. 5D2, and wherein and [X] represents cₒaₒ in accordance with the foregoing.

101. The composition or the kit according to any of the preceding items, wherein the inhibitor of RTEL1 is a single stranded antisense oligonucleotide capable of inhibiting the expression of RTEL1, comprising or consisting of CTttattataactTgaAtCTC (SEQ ID NO: 246); and wherein the inhibitor of FUBP1 is a single stranded antisense oligonucleotide capable of inhibiting the expression of FUBP1, which is selected from the group of antisense oligonucleotides comprising or consisting of:

(SEQ ID NO: 325)
CTTatGctttttatgGT,
(SEQ ID NO: 325)
CTTaTgctttttatgGT,
(SEQ ID NO: 326)
CTtATgctttttatgGTT,
(SEQ ID NO: 326)
CTtAtgctttttatgGTT,
(SEQ ID NO: 326)
CTtAtgctttttatGgTT,
(SEQ ID NO: 326)
CTtAtgctttttatGGTT,
(SEQ ID NO: 327)
GcttTttatggtTtCAC,
(SEQ ID NO: 328)
TATgcTttttatggtTTC,
(SEQ ID NO: 329)
AcCAAttttcatttCtAC
and
(SEQ ID NO: 330)
CcccataaccataGTC;

wherein capital letters are beta-D-oxy LNA nucleosides, lowercase letters are DNA nucleosides, all LNA C are 5-methyl cytosine, and all internucleoside linkages are phosphorothioate internucleoside linkages.

102. The composition or the kit according to item 101, wherein the inhibitor of RTEL1 is the conjugate consisting of 5′-GN2-C6ₒ[X]^{m}CₛTₛtₛtₛaₛtₛtₛaₛtₛaₛaₛcₛtₛTₛgₛaₛAₛtₛ^{m}CₛTₛ^{m}Cₛ, such as shown in FIG. 4, and
wherein the inhibitor of FUBP1 is a conjugate selected from the group consisting of

5′-GN2-C6ₒ[X]^{m}CₛTₛTₛaₛtₛGₛcₛtₛtₛtₛtₛtₛaₛtₛgₛGₛT,
as shown in FIG. 8,
5′-GN2-C6ₒ[X]^{m}CₛTₛTₛaₛTₛgₛcₛtₛtₛtₛtₛtₛaₛtₛgₛGₛT,
as shown in FIG. 9,
5′-GN2-C6ₒ[X]^{m}CₛTₛtₛAₛTₛgₛcₛtₛtₛtₛtₛtₛaₛtₛgₛGₛTₛT,
as shown in FIG. 10,
5′-GN2-C6ₒ[X]^{m}CₛTₛtₛAₛtₛgₛcₛtₛtₛtₛtₛtₛaₛtₛgₛGₛTₛT,
as shown in FIG. 11,
5′-GN2-C6ₒ[X]^{m}CₛTₛtₛAₛtₛgₛcₛtₛtₛtₛtₛtₛaₛtₛGₛgₛTₛT,
as shown in FIG. 12,
5′-GN2-C6ₒ[X]^{m}CₛTₛtₛAₛtₛgₛcₛtₛtₛtₛtₛtₛaₛtₛGₛGₛTₛT,
as shown in FIG. 13,
5′-GN2-C6ₒ[X]GₛcₛtₛtₛTₛtₛtₛaₛtₛgₛgₛtₛTₛtₛ^{m}CₛAₛ^{m}C,
as shown in FIG. 14,
5′-GN2-C6ₒ[X]TₛAₛTₛgₛcₛTₛtₛtₛtₛtₛaₛtₛgₛgₛtₛTₛTₛ^{m}C,
as shown in FIG. 15,
5′-GN2-C6ₒ[X]Aₛcₛ^{m}CₛAₛAₛtₛtₛtₛtₛcₛaₛtₛtₛtₛ^{m}CₛtAₛ^{m}C,
as shown in FIG. 16,
and
5′-GN2-C6ₒ[X]^{m}CₛcₛcₛcₛaₛtₛaₛaₛcₛcₛaₛtₛaₛGₛTₛ^{m}Cₛ;

wherein a capital letter represents a beta-D-oxy LNA nucleoside, a lower case letter represents a DNA nucleoside, wherein each LNA cytosine is 5-methyl cytosine, and ^{m}C is 5-methyl cytosine DNA, and wherein subscript s represents a phosphorothioate internucleoside linkage, and a subscript o represents a phosphodiester internucleoside linkage, and GN2-C6 is a residue of formula:

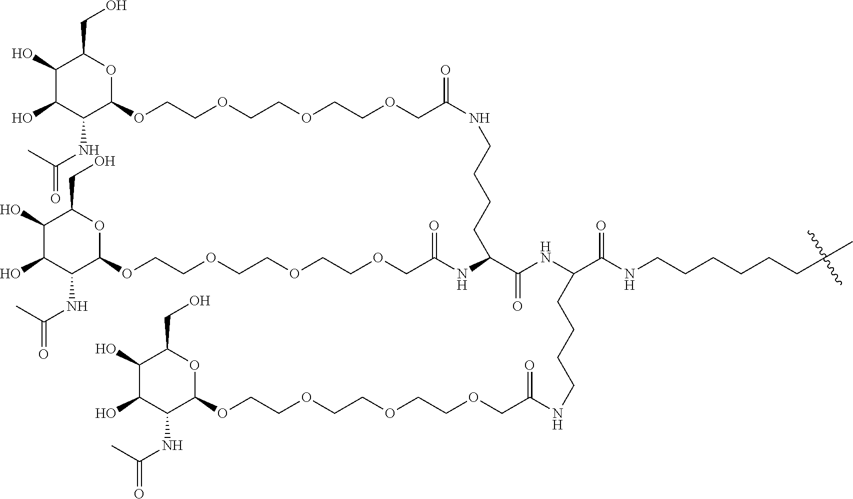 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

wherein the residue GN2-C6 is attached via a phosphodiester linkage at the 5′ end of the oligonucleotide, and/or wherein GN2-C6 is a tri-valent N-acetylgalactosamine (GalNAc) of FIG. 5D1 or FIG. 5D2, or a mixture of both, more preferably wherein GN2-C6 is a mixture of the tri-valent N-acetylgalactosamine (GalNAc) residues depicted in FIG. 5D1 or FIG. 5D2, and wherein and [X] represents cₒaₒ in accordance with the foregoing.

103. The composition or the kit according to any of the preceding items, wherein the inhibitor of RTEL1 is a single stranded antisense oligonucleotide capable of inhibiting the expression of RTEL1, comprising or consisting of CTttattataacttgaaTCTC (SEQ ID NO: 246); and
wherein the inhibitor of FUBP1 is a single stranded antisense oligonucleotide capable of inhibiting the expression of FUBP1, which is selected from the group of antisense oligonucleotides comprising or consisting of:

(SEQ ID NO: 325)
CTTatGctttttatgGT,
(SEQ ID NO: 325)
CTTaTgctttttatgGT,
(SEQ ID NO: 326)
CTtATgctttttatgGTT,
(SEQ ID NO: 326)
CTtAtgctttttatgGTT,
(SEQ ID NO: 326)
CTtAtgctttttatGgTT,
(SEQ ID NO: 326)
CTtAtgctttttatGGTT,
(SEQ ID NO: 327)
GcttTttatggtTtCAC,
(SEQ ID NO: 328)
TATgcTttttatggtTTC,
(SEQ ID NO: 329)
AcCAAttttcatttCtAC
and
(SEQ ID NO: 330)
CcccataaccataGTC;

preferably CcccataaccataGTC (SEQ ID NO: 330);
   wherein capital letters are beta-D-oxy LNA nucleosides, lowercase letters are DNA nucleosides, all LNA C are 5-methyl cytosine, and all internucleoside linkages are phosphorothioate internucleoside linkages.

104. The composition or the kit according to item 103, wherein the inhibitor of RTEL1 is the conjugate consisting of 5′-GN2-C6ₒ[X]^{m}CₛTₛtₛtₛaₛtₛtₛaₛtₛaₛaₛcₛtₛtₛgₛaₛaₛTₛ^{m}CₛTₛ^{m}Cₛ, and wherein the inhibitor of FUBP1 is a conjugate selected from the group consisting of

5′-GN2-C6ₒ[X]^{m}CₛTₛTₛaₛtₛGₛcₛtₛtₛtₛtₛtₛaₛtₛgₛGₛT,
as shown in FIG. 8,
5′-GN2-C6ₒ[X]^{m}CₛTₛTₛaₛTₛgₛcₛtₛtₛtₛtₛtₛaₛtₛgₛGₛT,
as shown in FIG. 9,
5′-GN2-C6ₒ[X]^{m}CₛTₛtₛAₛTₛgₛcₛtₛtₛtₛtₛtₛaₛtₛgₛGₛTₛT,
as shown in FIG. 10,
5′-GN2-C6ₒ[X]^{m}CₛTₛtₛAₛtₛgₛcₛtₛtₛtₛtₛtₛaₛtₛgₛGₛTₛT,
as shown in FIG. 11,
5′-GN2-C6ₒ[X]^{m}CₛTₛtₛAₛtₛgₛcₛtₛtₛtₛtₛtₛaₛtₛGₛgₛTₛT,
as shown in FIG. 12,
5′-GN2-C6ₒ[X]^{m}CₛTₛtₛAₛtₛgₛcₛtₛtₛtₛtₛtₛaₛtₛGₛGₛTₛT,
as shown in FIG. 13,
5′-GN2-C6ₒ[X]GₛcₛtₛtₛTₛtₛtₛaₛtₛgₛgₛtₛTₛtₛ^{m}CₛAₛ^{m}C,
as shown in FIG. 14,
5′-GN2-C6ₒ[X]TₛAₛTₛgₛcₛTₛtₛtₛtₛtₛaₛtₛgₛgₛtₛTₛTₛ^{m}C,
as shown in FIG. 15,
5′-GN2-C6ₒ[X]AₛCₛ^{m}CₛAₛAₛtₛtₛtₛtₛcₛaₛtₛtₛtₛ^{m}CₛtAₛ^{m}C,
as shown in FIG. 16,
and
5′-GN2-C6ₒ[X]^{m}CₛcₛcₛcₛaₛtₛaₛaₛcₛcₛaₛtₛaₛGₛTₛ^{m}Cₛ;

preferably 5′-GN2-C6ₒ[X]^{m}CₛcₛcₛcₛaₛtₛaₛaₛcₛcₛaₛtₛaₛGₛTₛ^{m}Cₛ; wherein a capital letter represents a beta-D-oxy LNA nucleoside, a lower case letter represents a DNA nucleoside, wherein each LNA cytosine is 5-methyl cytosine, and ^{m}C is 5-methyl cytosine DNA, and wherein subscript s represents a phosphorothioate internucleoside linkage, and a subscript o represents a phosphodiester internucleoside linkage, and GN2-C6 is a residue of formula:

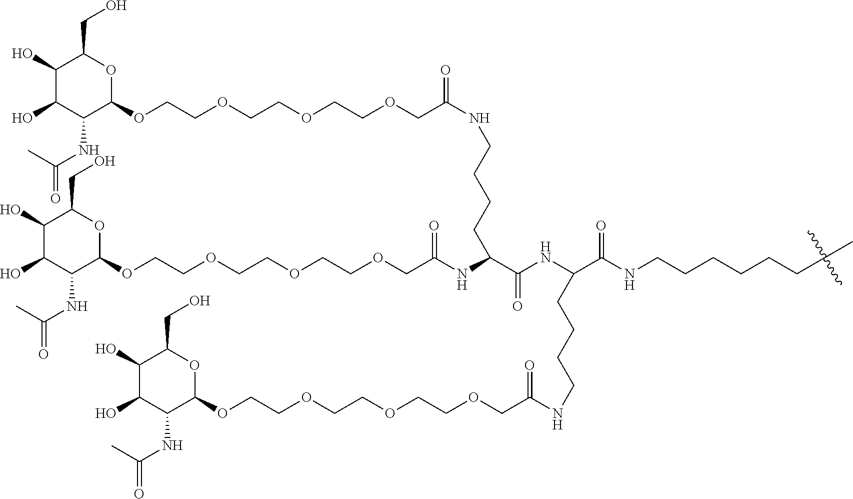 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

wherein the residue GN2-C6 is attached via a phosphodiester linkage at the 5′ end of the oligonucleotide, and/or wherein GN2-C6 is a tri-valent N-acetylgalactosamine (GalNAc) of FIG. 5D1 or FIG. 5D2, or a mixture of both, more preferably wherein GN2-C6 is a mixture of the tri-valent N-acetylgalactosamine (GalNAc) residues depicted in FIG. 5D1 or FIG. 5D2, and wherein and [X] represents cₒaₒ in accordance with the foregoing.

105. The composition or the kit according to any of the preceding items, for use in the treatment or prevention of a disease.

106. The composition or the kit according to any of the preceding items, for use in the treatment or prevention of a hepatitis B virus (HBV) infection.

107. An inhibitor of RTEL1, for use in the treatment or prevention of a disease, wherein the treatment or prevention further comprises the administration of an inhibitor of FUBP1.

108. An inhibitor of RTEL1, for use in the treatment or prevention of a hepatitis B virus (HBV) infection and/or cancer, preferably in a subject who is at risk of developing, who has developed, or has previously developed a HBV-associated hepatocellular carcinoma (HCC), wherein the treatment or prevention further comprises the administration of an inhibitor of FUBP1.

109. An inhibitor of RTEL1 for use according to any of item 107 or 108, wherein the inhibitor of RTEL1 is an inhibitor as defined in any of item 4 to 44.

110. An inhibitor of FUBP1, for use in the treatment or prevention of a disease, wherein the treatment or prevention further comprises the administration of an inhibitor of RTEL1.

111. An inhibitor of FUBP1, for use in the treatment or prevention of a hepatitis B virus (HBV) infection and/or cancer, preferably in a subject who is at risk of developing, who has developed, or has previously developed a HBV-associated hepatocellular carcinoma (HCC), wherein the treatment or prevention further comprises the administration of an inhibitor of RTEL1.

112. An inhibitor of FUBP1 for use according to any of item 110 or 111, wherein the inhibitor of FUBP1 is an inhibitor as defined in any of item 45 to 94.

113. A combination of an inhibitor of RTEL1 and an inhibitor of FUBP1, for use in the treatment or prevention of a disease.

114. A combination of an inhibitor of RTEL1 and an inhibitor of FUBP1, for use in the treatment or prevention of a disease; wherein the RTEL1 inhibitor is an inhibitor according to any of items 4 to 44.

115. A combination of an inhibitor of RTEL1 and an inhibitor of FUBP1, according to item 113 or 114; wherein the FUBP1 inhibitor is an inhibitor according to any of items 45 to 94.

116. A combination of an inhibitor of RTEL1 and an inhibitor of FUBP1, for use in the treatment or prevention of a hepatitis B virus (HBV) infection and/or cancer, preferably in a subject who is at risk of developing, who has developed, or has previously developed a HBV-associated hepatocellular carcinoma (HCC).

117. A combination of an inhibitor of RTEL1 and an inhibitor of FUBP1, for use according to item 116, wherein the RTEL1 inhibitor is an inhibitor according to any of items 4 to 44.

118. A combination of an inhibitor of RTEL1 and an inhibitor of FUBP1, for use according to item 116 or 117; wherein the FUBP1 inhibitor is an inhibitor according to any of items 45 to 94.

119. The composition or kit for use according to item 105 or 106; the inhibitor of RTEL1 for use according to any of items 107 to 109; the inhibitor of FUBP1 for use according to any of items 110 to 112; or the combination for use according to any of items 113 to 118, wherein the HBV infection is a chronic HBV infection.

120. The composition or kit for use according to item 105 or 106; the inhibitor of RTEL1 for use according to any of items 107 to 109; the inhibitor of FUBP1 for use according to any of items 110 to 112; or the combination for use according to any of items 113 to 118, wherein the RTEL1 inhibitor is capable of reducing cccDNA in an infected cell.

121. The composition or kit for use; the inhibitor of RTEL1 for use; the inhibitor of FUBP1 for use; or the combination for use according to item 120, wherein the cccDNA in an HBV infected cell is reduced by at least 60% when compared to a control.

122. A method for treating or preventing a disease comprising administering a therapeutically or prophylactically effective amount of an inhibitor of RTEL1, to a subject suffering from or susceptible to the disease, wherein the method further comprises the administration of an effective amount of an inhibitor of FUBP1.

123. A method for treating or preventing a disease comprising administering a therapeutically or prophylactically effective amount of an inhibitor of FUBP1, to a subject suffering from or susceptible to the disease, wherein the method further comprises the administration of an effective amount of an inhibitor of RTEL1.

124. A method for treating or preventing a disease comprising administering a combination of a therapeutically or prophylactically effective amount of an inhibitor of RTEL1 and a therapeutically or prophylactically effective amount of an inhibitor of FUBP1 to a subject suffering from or susceptible to the disease.

125. Use of an inhibitor of FUBP1 and an inhibitor of RTEL1, for the preparation of a medicament for treatment or prevention of hepatitis B virus (HBV) and/or cancer.

126. A method or use according to any of items 122 to 125, wherein the disease is a hepatitis B virus (HBV) infection and/or cancer.

127. A method or use according to any of items 122 to 126, wherein the disease is a chronic hepatitis B virus (HBV) infection.

128. An in vivo or in vitro method for modulating RTEL1 and FUBP1 expression in a target cell which is expressing RTEL1 and FUBP1, said method comprising administering an inhibitor of RTEL1 and an inhibitor of FUBP1; in an effective amount to said cell.

129. A method or use according to any of items 122 to 128, wherein the inhibitor of RTEL1 is an inhibitor as defined in any of items 4 to 44, or a pharmaceutical composition according to item 2.

130. A method or use according to any of items 122 to 129, wherein the inhibitor of FUBP1 is an inhibitor as defined in any of item 45 to 94, or a pharmaceutical composition according to item 2.

131. A compound comprising or consisting of an antisense oligonucleotide capable of reducing the expression of RTEL1 and FUBP1, wherein the antisense oligonucleotide is selected from the group of antisense oligonucleotides having a nucleotide sequence comprising or consisting of:

(SEQ ID NO: 348)
CCCCATAACCATAGTCCACTTTATTATAACTTGAATCTC;
(SEQ ID NO: 349)
CTTTATTATAACTTGAATCTCCACCCCATAACCATAGTC;
(SEQ ID NO: 350)
CTTATGCTTTTTATGGTTCATTACATACTCTGGTCAAA;
or
(SEQ ID NO: 351)
TTACATACTCTGGTCAAACACTTATGCTTTTTATGGTT;
preferably
(SEQ ID NO: 350)
CTTATGCTTTTTATGGTTCATTACATACTCTGGTCAAA;
or
(SEQ ID NO: 351)
TTACATACTCTGGTCAAACACTTATGCTTTTTATGGTT

132. A compound comprising or consisting of an antisense oligonucleotide capable of reducing the expression of RTEL1 and FUBP1, wherein the antisense oligonucleotide is selected from the group of antisense oligonucleotides comprising or consisting of:

^{m}CₛcₛcₛcₛaₛtₛaₛaₛcₛcₛaₛtₛaₛGₛTₛ^{m}Cₒcₒaₒ^{m}CₛTₛtₛtₛaₛtₛtₛaₛtₛaₛ
aₛcₛtₛtₛgₛaₛaₛTₛ^{m}CₛTₛ^{m}C;
^{m}CₛTₛtₛtₛaₛtₛtₛaₛtₛaₛaₛcₛtₛtₛgₛaₛaₛTₛ^{m}CₛTₛ^{m}Cₒcₒa_{c}^{m}Cₛcₛcₛaₛtₛ
aₛaₛcₛcₛaₛtₛaₛGₛTₛ^{m}C;
^{m}CₛTₛtₛAₛtₛgₛcₛtₛtₛtₛtₛtₛGₛgₛTₛTₒcₒaₒTₛTₛaₛcₛaₛtₛaₛcₛtₛcₛtₛ
gₛgₛtₛ^{m}CₛAₛAₛA;
or
TₛTₛaₛcₛaₛtₛaₛcₛtₛcₛtₛgₛgₛtₛ^{m}CₛAₛAₛAₒcₒaₒ^{m}CₛTₛtₛAₛtₛgₛcₛtₛtₛ
tₛtₛtₛaₛtₛGₛgₛTₛT;

wherein a capital letter represents a beta-D-oxy LNA nucleoside, a lower case letter represents a DNA nucleoside, wherein ^{m}C is 5-methyl cytosine LNA, and wherein subscript s represents a phosphorothioate internucleoside linkage, and a subscript o represents a phosphodiester internucleoside linkage.

133. A compound comprising or consisting of an antisense oligonucleotide capable of reducing the expression of RTEL1 and FUBP1, wherein the antisense oligonucleotide is selected from the group of antisense oligonucleotides comprising or consisting of:

5′-GN2-
C6ₒ[X]^{m}CₛCₛcₛcₛaₛtₛaₛaₛcₛcₛaₛtₛaₛGₛTₛ^{m}Cₒcₒaₒ^{m}CₛTₛtₛtₛaₛtₛtₛ
aₛtₛaₛaₛcₛtₛtₛgₛaₛaₛTₛ^{m}CₛTₛ^{m}C;
5′-GN2-
C6ₒ[X]^{m}CₛTₛtₛtₛaₛtₛtₛaₛtₛaₛaₛcₛtₛtₛgₛaₛaₛTₛ^{m}CₛTₛ^{m}Cₒcₒaₒ^{m}Cₛ
cₛcₛcₛaₛtₛaₛaₛcₛcₛaₛtₛaₛGₛTₛ^{m}C;
5′-GN2-C6ₒ[X]^{m}CₛTₛtₛAₛtₛgₛcₛtₛtₛtₛtₛtₛaₛtₛGₛgₛTₛTₒcₒaₒTₛ
Tₛaₛcₛaₛtₛaₛcₛtₛcₛtₛgₛgₛtₛ^{m}CₛAₛAₛA,
or
5′-GN2-C6ₒ[X]^{m}TₛTₛaₛcₛaₛtₛaₛcₛtₛcₛtₛgₛgₛtₛ^{m}CₛAₛAₛAₒcₒaₒ^{m}
CₛTₛtₛAₛtₛgₛcₛtₛtₛtₛtₛtₛₐₛtₛGₛgₛTₛT;

wherein a capital letter represents a beta-D-oxy LNA nucleoside, a lower case letter represents a DNA nucleoside, wherein ^{m}C is 5-methyl cytosine LNA, and wherein subscript s represents a phosphorothioate internucleoside linkage, and a subscript o represents a phosphodiester internucleoside linkage, and GN2-C6 is a residue of formula:

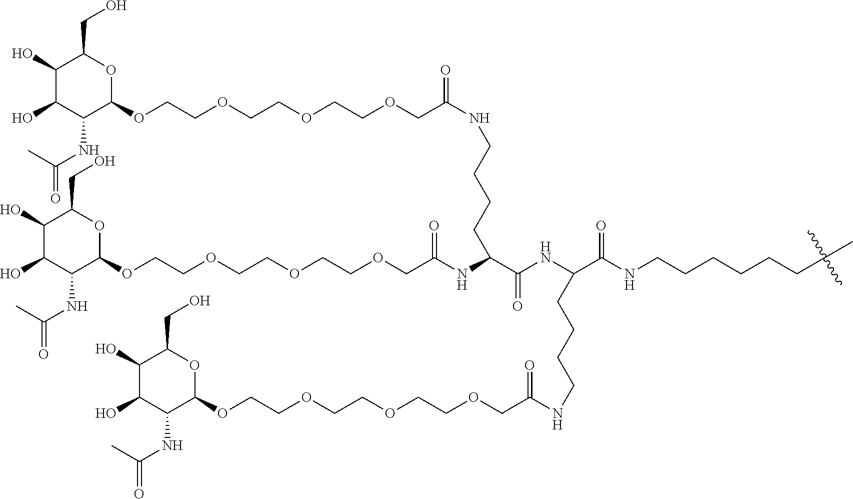 [SMILES: *CCCCCCNC(=O)C(CCCCNC(=O)COCCOCCOCCO[C@@H]1O[C@H](CO)[C@H](O)[C@H](O)[C@H]1NC(C)=O)NC(=O)[C@H](CCCCNC(=O)COCCOCCOCCO[C@@H]1O[C@H](CO)[C@H](O)[C@H](O)[C@H]1NC(C)=O)NC(=O)COCCOCCOCCO[C@@H]1O[C@H](CO)[C@H](O)[C@H](O)[C@H]1NC(C)=O]

wherein the residue GN2-C6 is attached via a phosphodiester linkage at the 5′ end of the oligonucleotide, and/or wherein GN2-C6 is a tri-valent N-acetylgalactosamine (GalNAc) of FIG. 5D1 or FIG. 5D2, or a mixture of both, more preferably wherein GN2-C6 is a mixture of the tri-valent N-acetylgalactosamine (GalNAc) residues depicted in FIG. 5D1 or FIG. 5D2, and wherein and [X] represents cₒaₒ in accordance with the foregoing.

134. A compound comprising or consisting of an antisense oligonucleotide capable of reducing the expression of RTEL1 and FUBP1, wherein the antisense oligonucleotide is selected from the group of antisense oligonucleotides having a sequence comprising or consisting of any of the HELM sequences set forth in Table 12D or Table 15B.

135. A compound according to any of items 131 to 134, for use in the treatment or prevention of a disease, preferably a hepatitis B virus (HBV) infection.

136. A compound according to any of items 131 to 134, for use in the treatment or prevention of a hepatitis B virus (HBV) infection and/or cancer, preferably in a subject who is at risk of developing, who has developed, or has previously developed a HBV-associated hepatocellular carcinoma (HCC).

137. A method for treating or preventing a disease, preferably a hepatitis B virus (HBV) infection and/or cancer, more preferably in a subject who is at risk of developing, who has developed, or has previously developed a HBV-associated hepatocellular carcinoma (HCC); comprising administering a therapeutically or prophylactically effective amount of a compound according to any of items 131 to 134.

138. Use of a compound according to any of items 13 to 134, for the preparation of a medicament for treatment or prevention of hepatitis B virus (HBV) and/or cancer.

139. An in vivo or in vitro method for modulating RTEL1 and FUBP1 expression in a target cell which is expressing RTEL1 and FUBP1, said method comprising administering a compound according to any of items 131 to 134 in an effective amount to said cell.

### EXAMPLES

### Example 1-Antisense Oligonucleotides Targeting RTEL1

### Materials and Methods

### Oligonucleotide Synthesis

Oligonucleotide synthesis is generally known in the art. Below is a protocol, which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

The coupling of β-cyanoethyl-phosphoramidites (DNA-A (Bz), DNA-G (ibu), DNA-C(Bz), DNA-T, LNA-5-methyl-C(Bz), LNA-A (Bz), LNA-G (dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphodiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

The crude compounds are purified by preparative RP-HPLC on a Phenomenex Jupiter® C18 10μ 150×10 mm column. 0.1 M ammonium acetate pH 8 and acetonitrile is used as buffers at a flow rate of 5 mL/min. The collected fractions are lyophilized to give the purified compound typically as a white solid.

### Abbreviations

DCI: 4,5-Dicyanoimidazole
DCM: Dichloromethane
DMF: Dimethylformamide
DMT: 4,4′-Dimethoxytrityl
THF: Tetrahydrofurane
Bz: Benzoyl
Ibu: Isobutyryl
RP-HPLC: Reverse phase high performance liquid chromatography

### Primary Human Hepatocytes (PXB-PHH)

Fresh primary human hepatocytes (PXB-PHH) harvested from humanized mice (uPA/SCID mice)-herein called PHH-were obtained from PhoenixBio Co., Ltd (Japan) in 96-well format and cultured in modified hepatocyte clonal growth medium (dHCGM). dHCGM is a DMEM medium containing 100 U/ml Penicillin, 100 μg/ml Streptomycin, 20 mM Hepes, 44 mM NaHCO₃, 15 μg/ml L-proline, 0.25 μg/ml Insulin, 50 nM Dexamethazone, 5 ng/ml EGF, 0.1 mM Asc-2P, 2% DMSO and 10% FBS (Ishida et al., 2015).

Cells were cultured at 37° C., in a humidified atmosphere with 5% CO₂. Culture medium was replaced every 2 days until harvest, except over the weekend.

### HBV Infection and Oligonucleotide Treatment (RTEL1)

PHH were incubated with HBV (purified from chronic hepatitis B (CHB) individuals) at multiplicity of infection (MOI) of 40 together with 4% PEG for 24 hr. The virus inoculum was removed the following day and cells were washed 3 times with PBS before addition of fresh medium. To allow for cccDNA establishment, compound treatment in PHH was started at day 3 post HBV infection. The cells were dosed in a 1:10 dilution step dose response manner starting at 10 μM. On Day 3, Day 5 and Day 7 post HBV infection, the cells were dosed with oligonucleotide compounds in a final volume of 100 μl/well of dHCGM Medium. 10 nM Entecavir (ETV) treatment was started at Day 5 post infection, ensuring real cccDNA measurement by qPCR, and medium including 10 nM ETV was changed every two days (except over the weekend) until cells were harvested at Day 16 post HBV infection. The experiments were performed in biological triplicates.

### HBV Infection and Oligonucleotide Treatment (FUBP1)

Upon arrival, PHH were infected with an MOI 110 using chronic patient-derived purified inoculum (genotype C) by incubating the PHH cells with HBV in 4% (v/v) PEG in PHH medium for 16 hours. The cells were then washed three times with PBS and cultured in a humidified atmosphere with 5% CO₂ in fresh PHH medium. Four days post-infection the cells were treated with FUBP1 LNAs (see Table 11) at a final concentration of 10 μM in duplicate or with PBS as no drug control (NDC). On the day of the treatment, the old medium was removed from the cells and replaced by 400 μl/well of fresh PHH medium. Per well, 100 μL of each FUBP1 LNA at 50 μM or PBS as NDC were added to the 400 μL PHH medium. The same treatment was repeated 3 times on days 4, 11 and 18 post-infection. Cell culture medium was changed with fresh one every three days on days 7, 14 and 21 post-infection.

### Real-Time PCR for Intracellular RTEL1 RNA

Total mRNA was extracted from the cells using a Qiagen BioRobot Universal System and the RNeasy 96 well Extraction Plates (RNeasy 96 BioRobot 8000 Kit (12)/Cat No. ID: 967152) according to the manufacturer's protocol. The mRNA expression levels were analyzed using Real-time PCR on the ABI QuantStudio™ 12k Flex. Beta-actin (ACT B) was quantified by qPCR using TaqMan Fast Advanced Master Mix (Life Technologies, cat no. 4444558) in technical duplicates. qPCR for RTEL1 gene was performed with the Fast SYBR™ Green Master Mix (Life Technologies, Cat. No 4385612). Results were normalized over the human ACT B endogenous control. The mRNA expression was analyzed using the comparative cycle threshold 2-ΔΔCt method normalized to the reference gene ACT B and to non-treated cells. Primers used for ACTB RNA and RTEL1 RNA quantification are listed in Table 16:

TABLE 16
ACT B and RTEL1 RNA qPCR primers

| Parameter | Direction | Primer Sequence | Seq ID No |
|---|---|---|---|
| RTEL1 | Fwd | 5′-CCATCCTGGACATTGAGGACT-3′ | 332 |
| | Rev | 5′-CAGGTTCCGGGACAGGTAGTA-3′ | 333 |

Housekeeping gene primers ACT B (VIC): Hs01060665_g1 (Thermo Fisher Scientific)

HBV cccDNA Quantification

DNA was extracted from HBV infected Primary Human Hepatocytes using an SDS Lysis Buffer (50 mM Tris pH8, 5 mM EDTA, 1% SDS). After lysing cells in 80 μl SDS lysis buffer, samples were frozen at −80° C. for minimum 2 hours. Samples were thawed at 37° C. and 1 μl of Proteinase K (cat no. AM25448 @ Ambion biosciences, 20 mg/mL Stock) was added to each well of the 96-well plate and samples were incubated at 56° C. for 30 minutes. After incubation, 3 volumes of ChIP DNA binding buffer from ZYMO Research Genomic DNA Clean & Concentrator kit (ZymoResearch, cat no. D4067) were added and DNA was purified following the manufacturer's protocol. DNA was eluted in 20 μl DNA Elution buffer and qPCR was performed using 2 μl DNA.

The cccDNA expression levels were quantified in technical duplicates using the comparative cycle threshold 2-ΔΔCt method. Quantitative real-time polymerase chain reaction measurements were performed on the QuantStudio 12K Flex PCR System (Applied Biosystems). Normalization was done to mitochondrial DNA (mitoDNA) and to non-treated cells as endogenous control using the Fast SYBR™ Green Master Mix (Life Technologies, Cat. No 4385612). Cycler settings were adjusted to incubation at 95° C. for 5 min, then 45 cycles of 95° C. for 1 sec and 60° C. for 35 sec. Primers used are listed Table 17 below (all probes in the chart are SYBR Green):

TABLE 17
cccDNA qPCR primers.

| Parameter | Direction | Primer Sequence | Seq ID No |
|---|---|---|---|
| cccDNA | Fwd | 5′-CGTCTGTGCCTTCTCATCTGC-3′ | 333 |
| mitochondrial | Rev | 5′-GCACAGCTTGGAGGCTTGAA-3′ | 334 |
| DNA | Fwd | 5′-CCGTCTGAACTATCCTGCCC-3′ | 335 |
| | Rev | 5′-GCCGTAGTCGGTGTACTCGT-3′ | 336 |

### Example 1.1—Effect of Antisense Oligonucleotides Targeting RTEL1 on RTEL1 RNA and on cccDNA in HBV Infected PHH Cells

The effects of RTEL1 knock-down on RTEL1 RNA and on cccDNA were tested using the oligonucleotide compounds from Table 6. PHH were cultured as described in the Materials and Methods section. HBV infected PHH cells were treated with the compounds from Table 6 as described above. Following the 16 days-treatment, RTEL1 mRNA and cccDNA were measured by qPCR as described above. The results are shown in Table 18 as % of the average no drug control (NDC) samples (i.e. the lower the value the larger the inhibition/reduction).

TABLE 18
Effect on cccDNA following knockdown of RTEL1 with LNA ASO

| | | | cccDNA | | | |
|---|---|---|---|---|---|---|
| CMP | SEQ | | % of | | RTEL % of | |
| ID | ID | | control | cccDNA | control | RTL1 |
| NO | NO | Compound | (Mean) | SD | (Mean) | SD |
| 243_1 | 243 | AATTttacatactctgGT | 28 | 13 | 19 | 3 |
| 244_1 | 244 | AAttttacatactctGGTC | 3 | 1 | 24 | 2 |
| 245_1 | 245 | TTacatactctggtCAAA | 21 | 6 | 35 | 11 |
| 246_1 | 246 | CTttattataactTgaAtCTC | 39 | 5 | 21 | 8 |

For Compounds: Capital letters represent LNA nucleosides (beta-D-oxy LNA nucleosides were used), all LNA cytosines are 5-methyl cytosine, lower case letters represent DNA nucleosides. All internucleoside linkages are phosphorothioate internucleoside linkages.

### Example 1.2—Testing In Vitro Efficacy of Antisense Oligonucleotides Targeting RTEL1 mRNA in Human MDA-MB-231 Cell Line at Different Concentrations for a Dose Response Curve

Human MDA-MB-231 cell lines were purchased from ATCC and maintained as recommended by the supplier in a humidified incubator at 37° C. with 5% CO₂. For assays, 3500 cells/well of were seeded in a 96 multi well plate in culture media. Cells were incubated for 24 hours before addition of oligonucleotides dissolved in PBS. Highest screening concentration of oligonucleotides: 50 μM and subsequent 1:1 dilutions in 8 steps. 3 days after addition of oligonucleotides, the cells were harvested. RNA was extracted using the PureLink™ Pro 96 RNA Purification kit (Thermo Fisher Scientific) according to the manufacturer's instructions and eluated in 50 μl water. The RNA was subsequently diluted 10 times with DNase/RNase free Water (Gibco) and heated to 90° C. for one minute.

For gene expressions analysis, One Step RT-qPCR was performed using qScript™ XLT One-Step RT-qPCR ToughMix®, Low ROX™ (Quantabio) in a duplex set up. The following TaqMan primer assays were used for qPCR: RTEL1_Hs00249668_m1 [FAM-MGB] and endogenous control GUSB_Hs99999908_m1 [VIC-MGB]. All primer sets were purchased from Thermo Fisher Scientific. IC50 determination was performed in GraphPad Prism7.04 from biological replicates n=2. The relative RTEL1 mRNA level at treatment with 50 M oligonucleotide is shown in Table 19 as percent of control (PBS treated samples).

TABLE 19
IC50 values and mRNA levels at Max KD

| | | | | | | mRNA | |
|---|---|---|---|---|---|---|---|
| | | | | | | level at | |
| SEQ | | CMP | | IC₅₀ in | | Max KD | |
| ID | | ID | | MDA- | | in MDA- | |
| NO | Motif | NO | Compound | MB-231 | SD | MB-231 | SD |
| 243 | aattttacatactctggt | 243_1 | AATTttacatactctgGT | 0.6 | 0.1 | 38 | 1 |
| 244 | aattttacatactctggtc | 244_1 | AAttttacatactctGGTC | 0.3 | 0.1 | 14 | 1 |
| 245 | ttacatactctggtcaaa | 245_1 | TTacatactctggtCAAA | 0.9 | 0.3 | 9 | 4 |
| 246 | ctttattataacttgaatctc | 246_1 | CTttattataactTgaAtCTC | 0.4 | 0.1 | 18 | 3 |

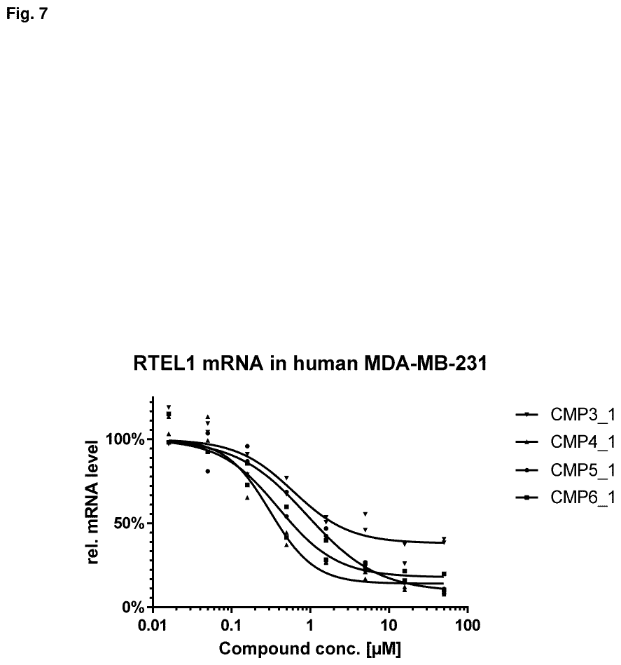

The compounds show very good efficacy and potency towards knockdown of human RTEL1 mRNA as the concentration response curves in human cell line MDA-MB-231 display, provided in FIG. 7.

### Example 2-Antisense Oligonucleotides Targeting FUBP1

### Introduction

Overexpression of and mutations in FUBP1 has been known to be associated with cancers for many years. In particular, strong overexpression of FUBP1 in human hepatocellular carcinoma (HCC) supports tumor growth and correlates with poor patient prognosis.

HBV cccDNA in infected hepatocytes is responsible for persistent chronic infection and reactivation, being the template for all viral subgenomic transcripts and pre-genomic RNA (pgRNA) to ensure both newly synthesized viral progeny and cccDNA pool replenishment via intracellular nucleocapsid recycling.

In WO 2019/193165, it was shown that FUBP1 is associated with cccDNA stability. This knowledge allows for the opportunity to destabilize cccDNA in HBV infected subjects which in turn opens the opportunity for a complete cure of chronically infected HBV patients. In the study, 2300 antisense oligonucleotides targeting human FUBP1 were screened. In this screening, compounds were identified which are particularly potent and effective to target human FUBP1. Specifically, nine alternating flank gapmer LNA oligonucleotides were identified which target a region within exon 14 of human FUBP1 and which conferred a strong down-regulation of human FUBP1 in vitro. Furthermore, one alternating flank gapmer LNA oligonucleotide was identified which targets a region within exon 20 of human FUBP1 and which conferred a strong down-regulation of human FUBP1 as well An overview on the identified nine compounds is provided in Table 12B above.

The target sequence of the identified compounds overlaps with the target sequence of CMP ID NO 294_1 and 295_1 as disclosed in WO 2019/193165. These two compounds inhibit FUBP1 in Hela cells to around ˜70% at 5 M. However, the nine identified compounds are clearly more efficacious, as they inhibit FUBP1 in Hela cells down to about ˜25% to 35% at 3.3 μM or to ˜27% at 5 μM (CMP ID NO: 329_1. In addition, they are more efficious in targeting FUBP1 in Hela cells than CMP ID NO 291_1, which is the best compound of WO 2019/193165 (see Example 2.1).

An overview on the prior art compounds 276_1, 291_1, 294_1, 295_1, 319_1 and 320_1 of WO 2019/193165 is provided in Table 20 below. The compounds are gapmers with uniform flanks. CMP ID NO: 291_1 was the best compound in PHH cells, CMP ID NO: 276_1 was the best compound in Hela cells. CMP ID NO 294_1 and 295_1 are the closest compounds for CMP ID Nos: 325_1, 325_2, 326_1, 326_2, 326_3, 326_4; 327_1 and 328_1. CMP ID NO 319_1 and 320_1 are the closest compounds for CMP ID NO: 329_1.

TABLE 20
list of control oligonucleotide compounds (as disclosed in WO 2019/193165)

| SEQ | | position on | | | CMP | |
|---|---|---|---|---|---|---|
| ID | | SEQ ID NO: 1 | | | ID | Oligonucleotide |
| NO | Motif sequence | Start | end | Design | NO | Compound |
| 276 | CCCATAACCATAGTCAT | 9142 | 9157 | 3-12- | 276_1 | CCCataaccatagtcAT |
| | | | | 2 | | |
| 291 | CCATTTCTTCCTATTACAA | 14783 | 14801 | 3-14- | 291_1 | CCAtttcttcctattacAA |
| | | | | 2 | | |
| 294 | GCTTTTTATGGTTTCACC | 16183 | 16200 | 1-15- | 294_1 | GctttttatggtttcaCC |
| | | | | 2 | | |
| 295 | ATGCTTTTTATGGTTTCAC | 16183 | 16202 | 1-17- | 295_1 | AtgctttttatggtttcaCC |
| | C | | | 2 | | |
| 319 | ATATTAACCTCCTATCAGT | 30511 | 30530 | 1-15- | 319_1 | AtattaacctcctatcAGT |
| | | | | 3 | | |
| 320 | AATATTAACCTCCTATCAG | 30512 | 30531 | 3-13- | 320_1 | AATattaacctcctatCA |
| | | | | 3 | | G |

For Compounds: Capital letters represent LNA nucleosides (beta-D-oxy LNA nucleosides were used), all LNA cytosines are 5-methyl cytosine, lower case letters represent DNA nucleosides. All internucleoside linkages are phosphorothioate internucleoside linkages

### Example 2.1—Testing In Vitro Efficacy of Antisense Oligonucleotides Targeting Human FUBP1 mRNA in Hela Cells

Antisense oligonucleotides targeting FUBP1 were tested for their ability to reduce FUBP1 mRNA expression in human Hela cells acquired from ECACC (Catalog No. 93021013).

Hela cells were grown in cell culturing media (EMEM [Sigma, cat. no M2279], supplemented with 10% Fetal Bovine Serum [Sigma, cat. no F7524], 2 mM Glutamine [Sigma, G7513], 0.1 mM NEAA [Sigma, M7145] and 0.025 mg/ml Gentamicin [Sigma, cat. no G1397]). Cells were trypsinized every 5 days, by washing with Phosphate Buffered Saline (PBS), [Sigma cat. no 14190-094] followed by addition of 0.25% Trypsin-EDTA solution (Sigma, T3924), 2-3 minutes incubation at 37° C., and trituration before cell seeding.

For experimental use, 2500 cells per well were seeded in 96 well plates (Nunc cat. no 167008) in 190 μL growth media. ASO dissolved in PBS was added approximately 24 hours after the cells were seeded to reach final custom concentrations. Cells were incubated for 3 days without any media change.

After incubation, cells were harvested by removal of media followed by addition of 125 μL RLT Lysis buffer (Qiagen 79216) and 125 μL 70% ethanol. RNA was purified according to the manufacturer's instruction (Qiagen RNeasy 96 kit) and eluted in a final volume of 200 μL DNase/RNase free Water (Gibco).

The RNA was heat shocked for 40 seconds at 90° C. to melt RNA: LNA duplexes, moved directly to ice and spun down before use. For one-step qPCR reaction qPCR-mix (qScript™XLE 1-step RT-qPCR TOUGHMIX®Low ROX from QauntaBio, cat. no 95134-500) was mixed with two IDT probes (final concentration 1×) to generate the mastermix. Taqman probes were acquired from IDT: FUBP1: Hs.PT.58.26883775 (primer-probe ratio 2, FAM) or ThermoFisher Scientific: GUSB: 4326320E. Mastermix (6 μL) and RNA (4 μL, 1-2 ng/μL) were then mixed in a qPCR plate (MICROAMP®optical 384 well, 4309849). After sealing, the plate was given a quick spin, 1000 g for 1 minute at RT, and transferred to a Viia™ 7 system (Applied Biosystems, Thermo), and the following PCR conditions used: 50° C. for 15 minutes; 95° C. for 3 minutes; 40 cycles of: 95° C. for 5 sec followed by a temperature decrease of 1.6° C./see followed by 60° C. for 45 sec. The data was analyzed using the QuantStudio™ Real_time PCR Software.

The qPCR data was captured and raw data quality control done in Quantstudio7 software.

The data were then imported into E-Workbook where a BioBook template was used to capture and analyze the data. The data were analyzed using the following steps:

1. Quantity calculated by the delta delta Ct method (Quantity=2\hat{ }(−Ct)*1000000000)

2. Quantity normalized to the calculated quantity for the housekeeping gene assay run in the same well. Relative Target Quantity=QUANTITY_target/QUANTITY_housekeeping

3. The RNA knockdown was calculated for each well by division with the mean of all PBS-treated wells on the same plate. Normalised Target Quantity=(Relative Target Quantity/[mean] Relative Target Quantity]_pbs_wells)*100

4. The final data are shown as a percentage of untreated (PBS) wells.

5. For concentration-response experiments, a curve was fitted from the RNA knockdown values (step 3-4) for each compound [either 8 or 10 concentrations, depending on the dilution model]. Curves are fitted using a 4 Parameter Sigmoidal Dose-Response Model in Biobook.

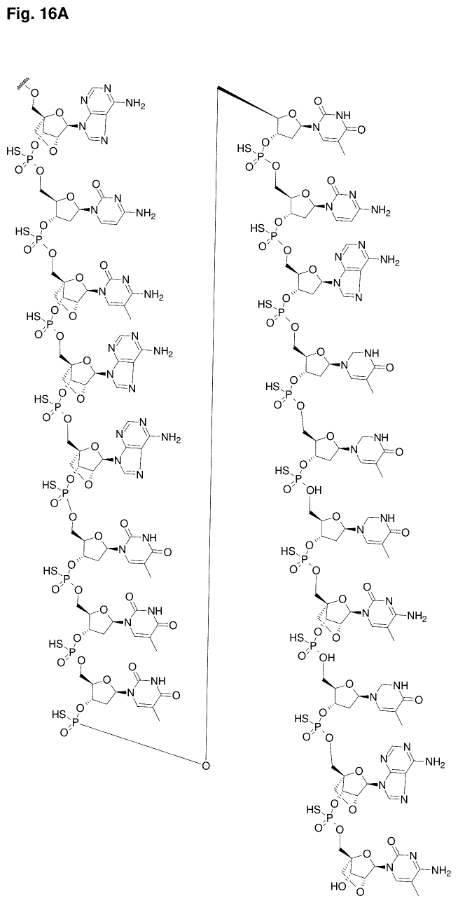

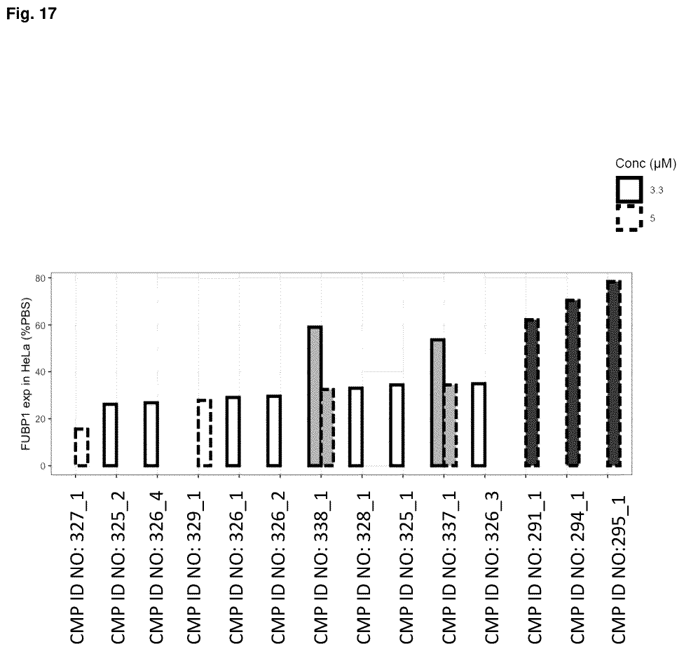

The relative FUBP1 mRNA expression levels are shown in Table 21 as % of control, i.e. the lower the value the larger the inhibition. Further, the results are shown in FIG. 17.

TABLE 21
In vitro efficacy of anti-FUBP1
compounds in Hela cells. FUBP1
mRNA levels are normalized to
GUSB and shown as % of control.

| CMP | FUBP1 Residual | |
|---|---|---|
| ID | mRNA level, % of ctrl | |
| NO | 3.3 μM | 5 μM |
| 325_1 | 34 | nd |
| 325_2 | 26 | nd |
| 326_1 | 29 | nd |
| 326_2 | 29 | nd |
| 326_3 | 34 | nd |
| 326_4 | 26 | nd |
| 327_1 | nd | 15 |
| 328_1 | 33 | nd |
| 329_1 | nd | 27.8 |
| 337_1** | 54 | 34 |
| 338_1** | 59 | 32 |
| 291_1* | nd | 62 |
| 294_1* | no | 70 |
| 295_1* | nd | 78 |
| 319_1* | nd | 68.4 |
| 320_1* | no | 51.4 |

*Control compounds, nd: not determined
**CMP ID NO: 337_1 is as follows: ATgctTtttatggtttCA (SEQ ID NO: 337), CMP ID NO: 338_1 is as follows: TTAtgctttttatggTTT (SEQ ID NO: 338), wherein capital letters are beta-D-oxy LNA nucleosides, lowercase letters are DNA nucleosides, all LNA C are 5-methyl cytosine, all internucleoside linkages are phosphorothioate internucleoside linkages. CMP ID NO: 337 targets nt 16185 to 16202 of SEQ ID NO: 247. CMP ID NO: 338 targets nt 16187 of 16204 of SEQ ID NO: 247.
Experiments with the control compounds were carried out separately

### Example 2.2—Testing In Vitro Efficacy of Antisense Oligonucleotides Targeting Human FUBP1 mRNA in Primary Human Hepatocytes (PXB-PHH)

Fresh primary human hepatocytes (PXB-PHH) harvested from humanized mice (uPA/SCID mice)-herein called PHH-were obtained from PhoenixBio Co., Ltd (Japan) in 96-well format and cultured in modified hepatocyte clonal growth medium (dHCGM). dHCGM is a DMEM medium containing 100 U/ml Penicillin, 100 μg/ml Streptomycin, 20 mM Hepes, 44 mM NaHCO₃, 15 μg/ml L-proline, 0.25 μg/ml Insulin, 50 nM Dexamethazone, 5 ng/ml EGF, 0.1 mM Asc-2P, 2% DMSO and 10% FBS (Ishida et al., 2015).

Cells were cultured at 37° C., in a humidified atmosphere with 5% CO₂. Culture medium was replaced 2 times per week until harvest.

Non-infected cells received a single treatment at 5 μM and were harvested 7 days later. In all treatments cells were dosed with oligonucleotide compounds in a final volume of 120 μl/well of dHCGM Medium. The experiments for RNA measurement were performed in biological duplicated.

Afterwards a real-time PCR for FUBP1 RNA was carried out. Total mRNA was extracted from the cells using a MagNA Pure robot and the MagNA Pure 96 Cellular RNA Large Volume Kit (Roche, #05467535001) according to the manufacturer's protocol. The mRNA expression levels were quantified in technical duplicates by qPCR using a QuantStudio 12K Flex (Applied Biosystems), the TaqMan RNA-to-CT 1-Step Kit (Applied Biosystems, #4392938), and human GusB endogenous control (Applied Biosystems, #Hs00939627_m1). The mRNA expression was analyzed using the comparative cycle threshold 2-ΔΔCt method normalized to the reference gene GusB and to non-treated cells. TaqMan primers used for GusB RNA and

FUBP1 RNA quantification are listed in the table 22 below:

TABLE 22
Primers for GusB RNA and
FUBP1 RNA quantification

| Parameter | Source |
|---|---|
| FUBP1 | ThermoFisher - Assay ID: Hs00900762_m1 |
| GusB | ThermoFisher - Assay ID: Hs00939627_m1 |

The relative FUBP1 mRNA expression levels of 8 compounds (CMP ID Nos: 325_1, 325_2, 326_1, 326_2, 326_3, 326_4; 327_1 and 328_1. CMP ID NO 319_1 and 320_1) in PXB-PHH cells are shown in Table 23 as % of control, i.e. the lower the value the larger the inhibition. The FUBP1 mRNA expression levels of CMP ID NO: 329_1) in PXB-PHH cells is analyzed in Example 2.3.

TABLE 23
In vitro efficacy of anti-FUBP1 compounds in PXB-PHH cells. FUBP1
mRNA levels are normalized to GUSB and shown as % of control.

| | | Rel. mRNA | | Rel. mRNA | |
|---|---|---|---|---|---|
| SEQ | CMP | level PXB- | | PXB-PHH | |
| ID | ID | PHH at | | at | |
| NO | NO | 25 μM | SD | 5 μM | SD |
| 325 | 325_1 | 37 | 3 | 46 | 4 |
| 325 | 325_2 | 33 | 4 | 45 | 4 |
| 326 | 326_1 | 31 | 1 | 52 | 15 |
| 326 | 326_2 | 21 | 1 | 43 | 2 |
| 326 | 326_3 | 33 | 1 | 49 | 5 |
| 326 | 326_4 | 26 | 0 | 41 | 0 |
| 327 | 327_1 | 33 | 1 | 59 | 5 |
| 328 | 328_1 | 22 | 2 | 60 | 38 |

### Conclusions Drawn from Examples 2.1 and 2.2

The data in Examples 2.1 and 2.2 show that targeting FUBP1 with an LNA ASO as shown in Table 12B leads to an efficient reduction of FUBP1.

### Example 2.3—Further Analysis of CMP IDs NO: 326_1 and 329_1

In the following, additional experiments with two of the nine identified compounds are described: CMP IDs NO: 326_1 and 329_1. In these experiments, the two compounds were compared to two prior compounds which gave the best results in WO 2019/193165

### Materials and Methods

### Primary Human Hepatocytes (PXB-PHH)

Fresh primary human hepatocytes (PXB-PHH) harvested from humanized mice (uPA/SCID mice)-herein called PHH-were obtained from PhoenixBio Co., Ltd (Japan) in 24-well format and cultured in modified hepatocyte clonal growth medium (dHCGM). dHCGM is a DMEM medium containing 100 U/ml Penicillin, 100 μg/ml Streptomycin, 20 mM Hepes, 44 mM NaHCO₃, 15 μg/ml L-proline, 0.25 μg/ml Insulin, 50 nM Dexamethazone, 5 ng/ml EGF, 0.1 mM Asc-2P, 2% DMSO and 10% FBS (Ishida et al., 2015).

Cells were cultured at 37° C., in a humidified atmosphere with 5% CO₂. Culture medium was replaced 2 times per week until harvest.

### ASOs Sequences and Compounds

Table 24 provides an overview on the compounds tested in Example 2.3:

TABLE 24
Human FUBP1 sequences targeted by the ASOs

| Description | Sequence | CMP ID |
|---|---|---|
| Compound | 5′- | 326_3* |
| according to | CTTATGCTTTTTATGGTT- | |
| invention | 3′ (SEQ ID NO: 326) | |
| Control | 5′- | 276_1** |
| compound | CCCATAACCATAGTCAT-3′ | |
| (best prior art | (SEQ ID NO: 276) | |
| compound in | | |
| Hela cells) | | |
| Compound | 5′- | 329_1* |
| according to | ACCAATTTTCATTTCTAC- | |
| invention | 3′ (SEQ ID NO: 329) | |
| Control | 5′- | 291_1** |
| compound | CCATTTCTTCCTATTACAA- | |
| (best prior art | 3′ (SEQ ID NO: 291) | |
| compound in | | |
| PHH cells) | | |

*see Table 12B, compounds according to the invention
**see Table 20: control compounds as disclosed in WO 2019/193165

### HBV Infection and Oligonucleotide Treatment

Upon arrival, PHH were infected with an MOI 110 using chronic patient-derived purified inoculum (genotype C) by incubating the PHH cells with HBV in 4% (v/v) PEG in PHH medium for 16 hours. The cells were then washed three times with PBS and cultured in a humidified atmosphere with 5% CO₂ in fresh PHH medium. Four days post-infection the cells were treated with FUBP1 LNAs (see Table 24) at a final concentration of 10 μM in duplicate or with PBS as no drug control (NDC). On the day of the treatment, the old medium was removed from the cells and replaced by 400 μl/well of fresh PHH medium. Per well, 100 UL of each FUBP1 LNA at 50 UM or PBS as NDC were added to the 400 μL PHH medium. The same treatment was repeated 3 times on days 4, 11 and 18 post-infection. Cell culture medium was changed with fresh one every three days on days 7, 14 and 21 post-infection.

Real-Time PCR for Intracellular HBV pgRNA and FUBP1 mRNA

Following cell viability determination the cells were washed with PBS once. Total RNA was extracted from the cells using a MagNA Pure robot and the MagNA Pure 96 Cellular RNA Large Volume Kit (Roche, #05467535001) according to the manufacturer's protocol. The FUBP1 mRNA and the viral pgRNA expression levels were quantified in technical duplicates by qPCR using a QuantStudio 12K Flex (Applied Biosystems). the TaqMan RNA-to-CT 1-Step Kit (Applied Biosystems, #4392938), and human GusB endogenous control (Applied Biosystems, #Hs00939627_m1) have been used. The FUBP1 mRNA and the viral pgRNA relative expressions were analyzed using the comparative cycle threshold 2-ΔΔCt method normalized to the reference gene GusB and non-treated cells. TaqMan primers used for GusB RNA, FUBP1 RNA and HBV pgRNA quantifications are listed in Table 25.

TABLE 25
TaqMan primers used for GusB gene,
FUBP1 RNA and HBV pgRNA quantifications

| Parameter | Source |
|---|---|
| FUBP1 | ThermoFisher - Assay ID: Hs00900762_m1 |
| HBV pgRNA | custom: AILJKX5 |
| GusB | ThermoFisher - Assay ID: Hs00939627_m1 |

### Results

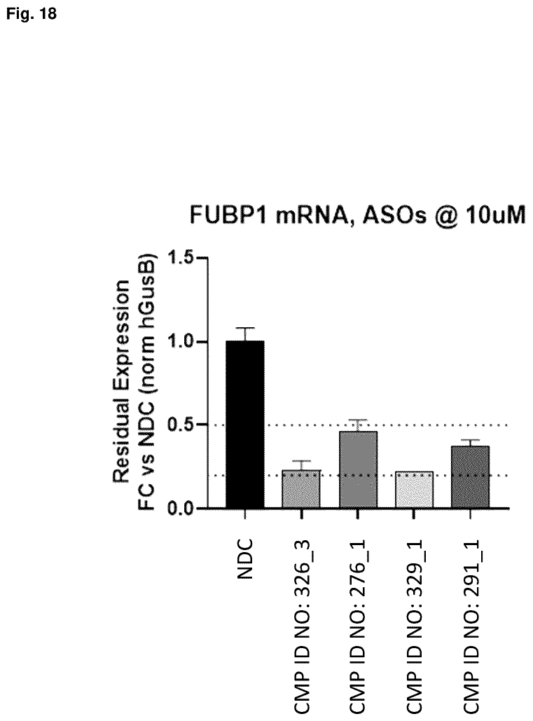

The results are shown in Table 26 and FIG. 18. As can be derived from the table 26 and FIG. 18, both compounds of the invention (CMP ID NO: 326_3 and 329_1) reduce target mRNA expression by about 80% compared to the NDC. Their effect on the FUBP1 mRNA level is much stronger than the effect of the prior art compounds.

TABLE 26
In vitro efficacy of anti-FUBP1 compounds in PXB-PHH cells. FUBP1
mRNA levels are normalized to GUSB and shown as % of control.

| | | Best naked Prio | | | | Best naked Prio | |
|---|---|---|---|---|---|---|---|
| | | Art in HeLa | | | | Art in PHH | |
| CMP ID 326_3 | | CMP ID 276_1 | | CMP ID 329_1 | | CMP ID 291_1 | |
| Residual | | Residual | | Residual | | Residual | |
| Expression | | Expression | | Expression | | Expression | |
| Rel to NDC | | Rel to NDC | | Rel to NDC | | Rel to NDC | |
| (=1) | SD | (=1) | SD | (=1) | SD | (=1) | SD |
| 0.23 | 0.04 | 0.46 | 0.05 | 0.22 | 0.00 | 0.38 | 0.03 |

### Example 3-Antisense Oligonucleotides Targeting RTEL1-In Vivo

### Materials and Methods

### Animals

Chimeric mice with humanized livers (PXB-mice) were generated from urokinase-type plasminogen activator-cDNA/severe combined immunodeficiency mice injected with human hepatocytes. (Tateno C, Kawase Y, Tobita Y, et al. Generation of novel chime-ric mice with humanized livers by using hemizygous cDNA-uPA/SCID mice. PLOS One. 2015; 10: e0142145.)

Candidate animals assigned to the study based on their body weight, overall health, serum h-Alb and HBV DNA concentrations. Mice with unexpected abnormalities such as weight loss of more than 20% of the initial body weight, moribundity, death or spontaneous tumor formation during the study were removed from the respective groups.

TABLE 27
Animal groups and compound dosing

| | | | Test | | Dose | | | |
|---|---|---|---|---|---|---|---|---|
| | | No. of | compound | Level | Conc. | Vol. | | |
| Group | Strain | mice | (SEQ ID NO) | (mg/kg) | (mg/kg) | (ml/kg) | Route | Frequency |
| 1 | PXB (HBV C- | 5 | Vehicle | 0 | 0 | 10 | s.c. | QD, 8 times, |
| | infected) | | | | | | | Days 0, 7, 14, |
| 2 | PXB (HBV C- | 5 | 330_1 | 10 | 1 | | | 21, 28, 35, 42, |
| | infected) | | | | | | | 49 |
| 3 | PXB (HBV C- | 5 | 246_2 | 10 | 1 | | | |
| | infected) | | | | | | | |
| 4 | PXB (HBV C- | 5 | 330_1 | 10 | 1 | | | |
| | infected) | | 246_2 | 10 | 1 | | | |

The compounds were administered by injection into the cervical subcutaneous tissue on the upper back on Days 0, 7, 14, 21, 28, 35, 42, 49 using disposable 1.0 mL syringes with permanently attached needles (Terumo Corporation, Tokyo, Japan).

### Blood Collection and Serum Separation

Seventy-five microliters (75 μL) of blood were collected from the subject animals under isoflurane (ISOFLURANE Inhalation Solution [Pfizer], Mylan, Osaka, Japan) anesthesia via the retro-orbital plexus/sinus using Intramedic™ Polyethylene Tubing (Becton, Dickinson and Company, NJ, USA) at each time point. At the terminal time point the animals were anesthetized with isoflurane anesthesia, blood collected from each animal via the heart after which the animals were sacrificed by cardiac puncture and exsanguination. Blood samples are incubated at room temperature for 5 minutes to coagulate, centrifuged at 13200×g, 4° C. for 3 minutes to obtain serum. The serum samples are stored at −80° C.

### Tissue Sample Preparation

At sacrifice whole livers from all animals are harvested, cut into pieces and snap-frozen with liquid nitrogen.

### Serum HBV DNA Quantification

Serum from an HBV-infected PXB-mouse was quantified by digital PCR and used as HBV standard by diluting with an appropriate volume of the HBV Pretreatment Solution for HBV DNA in Serum (KUBIX HBV qPCR Kit, KUBIX Inc., Hakusan, Japan) to make a target dilution series.

Samples to be analyzed were mixed with an appropriate volume of the HBV Pre-treatment Solution for HBV DNA in Serum, heated for 5 minutes at 98° C. and then analyzed by qPCR. The real-time qPCR was performed using the KUBIX HBV qPCR Kit (KUBIX Inc.) and CFX96 Touch™ Real-Time PCR Detection System (Bio-Rad Laboratories, Inc., Hercules, CA, USA). Twenty microliters (20 μL) of the HBV 2×PCR Solution was added to 20 μL of the heated sample. The initial activation was conducted at 95° C. for 2 minutes. Subsequent PCR amplification consisted of 45 cycles of denaturation at 95° C. for 5 seconds, and annealing and extension at 54° C. for 30 seconds per cycle in a CFX96 Touch™ Real-Time PCR Detection System. Average serum HBV DNA levels were calculated from the two technical replicates.

### Serum Hepatitis B Surface Antigen (HBsAg) and Hepatitis B e Antigen (HBeAg) Quantification

Serum HBsAg and HBeAg concentrations were determined by SRL, Inc. (Tokyo, Japan) using the Chemiluminescent Enzyme Immuno Assay (CLEIA) developed by Fujirebio (LUMIPULSE HBsAg-HQ, LUMIPULSER Presto II and LUMIPULSE HBeAg, LUMIPULSE® Presto II).

Total DNA Extraction from Liver Tissues

Mouse livers were homogenized in 1 ml total DNA extraction buffer (50 mM Tris pH8, 5 mM EDTA, 150 mM NaCl, 1% SDS) for 20 sec at 6000 rpm at RT. To each sample 2.5 μl RNase Cocktail (Invitrogen, #AM2286) was added, mixed and incubated at RT for 30 min. Protein digestion was done with 1 ul (20 μg) Proteinase K (Ambion, #AM2546, 20 mg/ml) at 56 C for 2 h at 300 rpm. Samples were spun down at 13,000 g for 3 min and the supernatant transferred to a fresh tube. The DNA was extracted 2-3 times with UltraPure Buffer-Saturated Phenol (Life Technologies, #15513-039) and once with UltraPure Phenol: Chloroform: Isoamyl Alcohol (25:24:1) (Life Technologies, #15593-031) by adding 1 ml of the reagent, mixing, spining at full speed for 15 min at 4° C. and transferring the aqueous solution to a new tube. DNA is then precipitated with 1 ml 100% EtOH and 40 μl 3M NaAc (SigmaAldrich, #S7899) at-20 C overnight. The DNA was pelleted at 4° C. max speed for 15 min, washed with 1 ml 70% EtOH and air-dried completely. Resuspension was in 50 μl 10 mM Tris-HCl, pH8. DNA was quantified by NanoDrop, adjusted to 1.5 μg/ul DNA and analyzed by Southern blot and qPCR.

### Southern Blot

DNA was loaded into a 1% agarose gel, and separated for 3.5 h at 50V by gel electrophoresis. As size marker DIG-labelled DNA Molecular Weight Marker VII (Sigma, #11669940910) was used. The gel was incubated in 500 mL freshly prepared 0.2 M HCl for 10 min. DNA denaturation was conducted with denaturation buffer (0.5 M NaOH, 1.5 M NaCl) for 30 min. The gel was then neutralized in 500 mL neutralizing buffer (0.5 M Tris-HCl PH 7.5, 1.5 M NaCl) and finally incubated in 500 mL UltraPure 20×SSC buffer (Life Technologies, #15557-036) and for 30 minutes each.

DNA was transferred to a Hybond-XL membrane (GE Healthcare, #RPN2020S) by capillary transfer in 20×SSC buffer overnight. The DNA was then UV-crosslinked to the membrane at 1800×100 uJ/cm2 once and allowed to dry. After pre-hybridization in 20 mL DIG Easy Hyb buffer (Roche, #11603558001) at 37° C. for 1 hour, the HBV specific DIG labelled DNA probe is denatured for 5 min at 100° C. and incubated on the membrane in 8 ml fresh DIG Easy Hyb buffer at 37° C. overnight. The DIG-labelled HBV DNA probe was prepared using DIG PCR probe kit (Sigma #11636090910) with forward (5-GTTTTTCACCTCTGCCTAATCATC-3; SEQ ID NO:339) and reverse primers (5-GCAAAAAGTTGCATGGTGCTGGT-3; SEQ ID NO:340) and a HBV GtC containing plasmid as a template. The qPCR was run with 95° C. for 5 min, followed by 40 cycles of 95° C. for 15 sec, 58° C. for 30 sec, 68° C. for 3 min and 15 sec, followed by 68° C. for 5 min and hold at 12° C.

Excess probe is washed away in 50 ml SSC wash buffer I (2×SSC, 0.1% SDS) twice for 5 min, then with 50 ml SSC wash buffer II (0.5×SSC, 0.1% SDS) twice for 15 min at 65° C.

DIG detection is then conducted using the DIG Wash and Block Buffer Set (Roche, #11585762001) according to the instructions. Anti-DIG antibody is diluted 1: 20′000 in 40 mL and incubated on the membrane for 60 min, following two washes with 15 min in 100 mL Wash buffer and 5 min incubation in 50 mL Detection buffer.

For luminescent detection 2-3 ml CDP-Star with NitroBlock is used and the image captured with the Fusion Fx (VILBER) and bands quantified with the ImageStudio lite software.

Intrahepatic HBV DNA Quantification by qPCR

The cccDNA and total HBV DNA levels were determined by qPCR using the TaqMan Fast Advanced Master Mix (Applied Biosystems, #4444557) with the primers HBB (ThermoFischer, #Hs00758889_s1, VIC), Total HBV (ThermoFischer, #Pa03453406_s1 P, S/P, FAM) and cccDNA primers (forward: CCGTGTGCACTTCGCTTCA, SEQ ID NO:341; reverse: GCACAGCTTGGAGGCTTGA, SEQ ID NO: 342; probe: FAM-CATGGAGACCACCGTGAACGCCC-5NFQ, SEQ ID NO:343). The qPCR was run on a QuantStudio 12K Flex Cycler with standard settings for Fast heating block (95° C. for 20 seconds, then 40 cycles with 95° C. for 1 second and 60° C. for 20 seconds, with 10 μl reaction volume). The HBB CT values were used to normalize the cccDNA and total HBV DNA CTS vlaues, calculate ddCTs and fold changes using the 2\hat{ }-ddCT method.

RNA Extraction from Liver Tissues

Tissue sections were lysed and homogenized in 1400 μl MagNaPure LC RNA Isolation Tissue buffer (Roche, #03604721001) using 2 mL MagNA Lyser Green Beads (Roche, #03358941001) and homogenizing at 6000 rpm for 20 sec and incubating at RT for 30 min. The homogenates were centrifuged 3 minutes at 13000 rpm. RNA was extracted from the supernatants via the MagNa Pure 96 (Roche) using the kit Cellular RNA Large Volume Kit (Roche, ##05467535001), the protocol used is “RNA Tissue FF Standard LV 3.1” and elution is done in 50 ul.

RNA concentrations were measured by NanoDrop and 1 μg RNA transcribed to cDNA using SuperScript™ III First-Strand Synthesis SuperMix for qRT-PCR (Invitrogen, #11752250) in 20 ul volume according to the manufacturer's protocol. The cDNA is diluted with water 1:3 by adding 40 ul H2O and analyzed by qPCR using the TaqMan Fast Advanced Master Mix (Applied Biosystems, #4444557) in a total volume of 10 ul per well. Taqman primer assays used are: pgRNA (probe_sequence GAGGCAGGTCCCCTAGAAGA; SEQ ID NO:344-FAM labelled, fwd_sequence GGAGTGTGGATTCGCACTCCT, SEQ ID NO:345; rev_sequence AGATTGAGATCTTCTGCGAC, SEQ ID NO: 346), FUBP1 (ThermoFischer, #Hs0090076_m1, FAM), GUSB (ThermoFischer, #Hs00939627_m1, VIC), RTEL1 (ThermoFischer, #Hs02568623_s1, FAM), Total HBV (ThermoFischer, #Pa03453406_s1 P, S/P, FAM). The qPCR was run the qPCR on QuantStudio Cycler with standard settings for Fast heating block (95° C. for 20 seconds, then 40 cycles with 95° C. for 1 second and 60° C. for 20 seconds, 10 μl reaction volume). GUSB CT values were used to normalize the target CTs, calculate ddCT and fold changes.

### Results

Intrahepatic HBV DNA levels were assessed by Southern blotting and semi-quantified by qPCR.

Southern blotting reveals a reduction of the cccDNA and total HBV DNA in the FUBP1 and RTEL1 LNA mono-treatment arms which is further enhanced in the FUBP1+RTEL1 combination arm (see FIG. 19).

TABLE 28
Southern blot band relative band intensities (FIG. 19).

| Mouse group | Signal in % | Average | SEM | N |
|---|---|---|---|---|
| Vehicle | >100 | 100 | 2.93 | 3 |
| | 94.15 | | | |
| | >100 | | | |
| | 103.21 | | | |
| | 102.64 | | | |
| FUBP1 | 60.73 | 52.88 | 11.07 | 4 |
| | 76.73 | | | |
| | 24.03 | | | |
| | 50.03 | | | |
| RTEL1 | 47.41 | 71.85 | 12.22 | 3 |
| | 83.51 | | | |
| | 84.62 | | | |
| FUBP1 + RTEL1 | 27.35 | 36.63 | 8.35 | 5 |
| | 22.93 | | | |
| | 33.52 | | | |
| | 69.30 | | | |
| | 30.07 | | | |

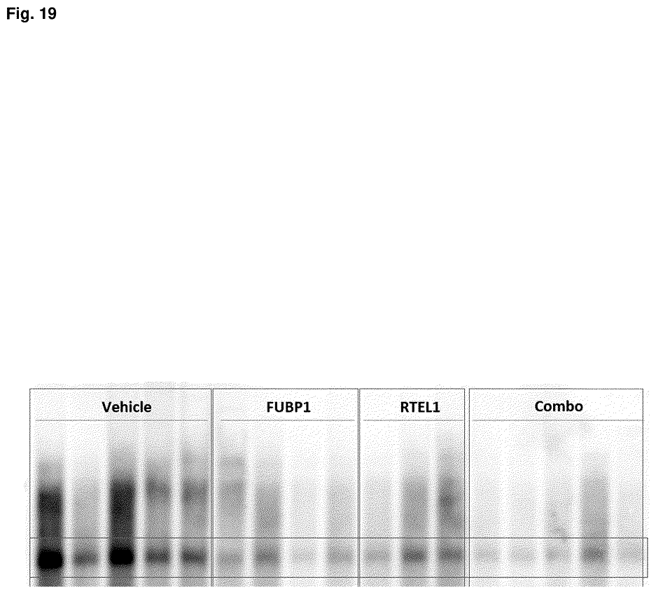

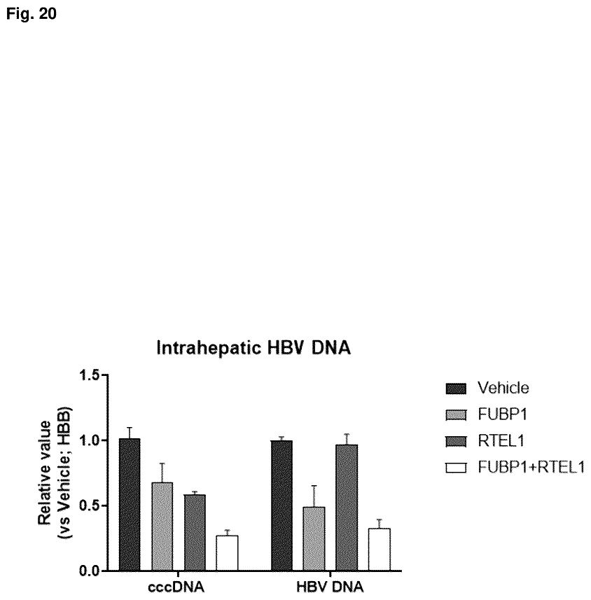

In the qPCR single compound administration showed cccDNA reductions by approximately 32% for FUBP1 LNA and 41% by RTEL1 LNA (FIG. 20 and Table 29). The dual combination of FUBP1 and RTEL1 LNAs showed even higher efficacy with a reduction of 73%. Total HBV DNA levels were also significantly reduced by 51% with FUBP1 LNA, whereas RTEL1 LNA did not impact the total HBV DNA levels. The combination of FUBP1 and RTEL1 LNA, on the other hand, lead to a decrease of 68%.

TABLE 29
Semi quantification of intrahepatic cccDNA
and total DNA levels by qPCR (see FIG. 19)

| | Vehicle | | | FUBP1 | | | RTEL1 | | | FUBP1 + RTEL1 | | |
|---|---|---|---|---|---|---|---|---|---|---|---|---|
| | Mean | SEM | N | Mean | SEM | N | Mean | SEM | N | Mean | SEM | N |
| cccDNA | 1.015 | 0.087 | 5 | 0.681 | 0.145 | 4 | 0.588 | 0.023 | 3 | 0.273 | 0.043 | 5 |
| HBV DNA | 1.002 | 0.029 | 5 | 0.494 | 0.162 | 4 | 0.973 | 0.078 | 3 | 0.329 | 0.067 | 5 |

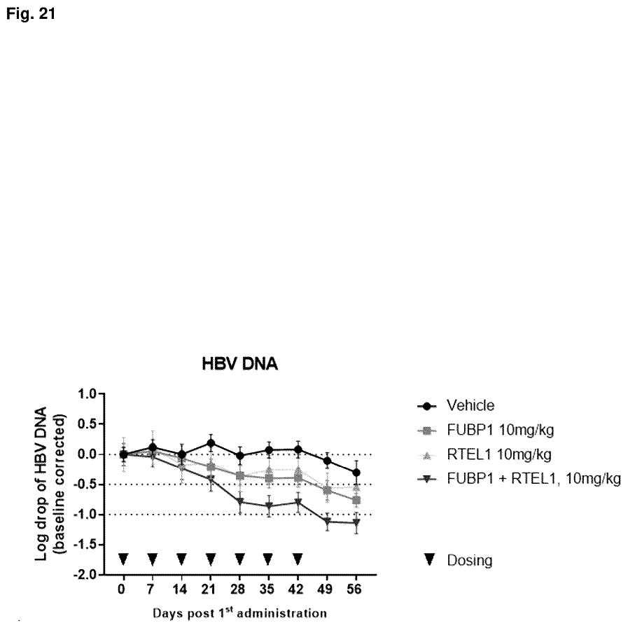

Baseline corrected serum HBV DNA levels showed a progressive decline of HBV DNA throughout treatment with both mono as well as the dual combination arms (FIG. 21 and Tables 30 and 31). While the mono treatment arms reached a log drop of 0.46+/−0.23 for the FUBP1 LNA and 0.24+/−0.31 for the RTEL1 LNA, the dual combination reduced the HBV DNA by 0.84+/−0.27 log below the vehicle control.

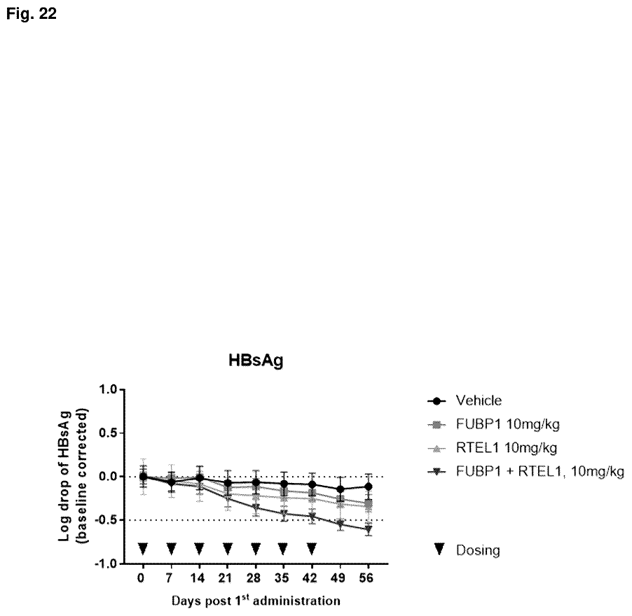

Likewise baseline corrected serum HBsAg levels showed a progressive decline throughout treatment with both mono as well as the dual combination arms (FIG. 22 and Tables 32 and 33). While the mono treatment arms reached a log drop of 0.35+/−0.18 for the FUBP1 LNA and 0.31+/−0.10 for the RTEL1 LNA, the dual combination reduced the HBV DNA by 0.61+/−0.07 log below the vehicle control.

For baseline corrected serum HBeAg level the decline is more subtle, however, a continuous reduction by treatment with the single compounds and the dual combination arm (FIG. 23 and Tables 34 and 35) can be detected. While the mono treatment arms reached a log drop of 0.16+/−0.03 for the FUBP1 LNA and 0.12+/−0.18 for the RTEL1 LNA, the dual combination reduced the HBV DNA by 0.26+/−0.15 log below the vehicle control.

TABLE 30
Serum HBV DNA kinetic (baseline corrected)

| | | | RTEL1 | | FUBP1 | | FUBP1 + RTEL1, | |
|---|---|---|---|---|---|---|---|---|
| | Vehicle | | 10 mg/kg | | 10 mg/kg | | 10 mg/kg | |
| Day | Mean | SEM | Mean | SEM | Mean | SEM | Mean | SEM |
| 0 | 0.000 | 0.120 | 0.000 | 0.283 | 0.000 | 0.114 | 0.000 | 0.183 |
| 7 | 0.121 | 0.124 | 0.115 | 0.278 | 0.054 | 0.130 | −0.042 | 0.167 |
| 14 | 0.004 | 0.167 | −0.170 | 0.307 | −0.062 | 0.136 | −0.234 | 0.188 |
| 21 | 0.191 | 0.141 | −0.146 | 0.260 | −0.210 | 0.137 | −0.421 | 0.191 |
| 28 | −0.018 | 0.147 | −0.361 | 0.263 | −0.354 | 0.165 | −0.794 | 0.176 |
| 35 | 0.077 | 0.136 | −0.254 | 0.238 | −0.399 | 0.163 | −0.863 | 0.177 |
| 42 | 0.084 | 0.137 | −0.251 | 0.257 | −0.392 | 0.155 | −0.801 | 0.168 |
| 49 | −0.103 | 0.134 | −0.556 | 0.242 | −0.600 | 0.161 | −1.122 | 0.147 |
| 56 | −0.303 | 0.201 | −0.540 | 0.241 | −0.763 | 0.117 | −1.141 | 0.174 |

TABLE 31
Serum HBV DNA kinetic normalized to the vehicle
control group (baseline corrected)

| | | | RTEL1 | | FUBP1 | | FUBP1 + RTEL1, | |
|---|---|---|---|---|---|---|---|---|
| | Vehicle | | 10 mg/kg | | 10 mg/kg | | 10 mg/kg | |
| Day | Mean | SEM | Mean | SEM | Mean | SEM | Mean | SEM |
| 0 | 0.000 | 0.169 | 0.000 | 0.307 | 0.000 | 0.166 | 0.000 | 0.219 |
| 7 | 0.000 | 0.175 | −0.006 | 0.304 | −0.067 | 0.180 | −0.163 | 0.208 |
| 14 | 0.000 | 0.236 | −0.174 | 0.350 | −0.066 | 0.215 | −0.238 | 0.252 |
| 21 | 0.000 | 0.200 | −0.338 | 0.296 | −0.402 | 0.197 | −0.613 | 0.238 |
| 28 | 0.000 | 0.208 | −0.343 | 0.301 | −0.337 | 0.221 | −0.777 | 0.229 |
| 35 | 0.000 | 0.192 | −0.331 | 0.274 | −0.475 | 0.212 | −0.940 | 0.223 |
| 42 | 0.000 | 0.194 | −0.335 | 0.291 | −0.476 | 0.207 | −0.885 | 0.216 |
| 49 | 0.000 | 0.189 | −0.452 | 0.276 | −0.497 | 0.209 | −1.018 | 0.199 |
| 56 | 0.000 | 0.284 | −0.237 | 0.314 | −0.460 | 0.233 | −0.838 | 0.266 |

TABLE 32
Serum HBsAg kinetic (baseline corrected)

| | | | RTEL1 | | FUBP1 | | FUBP1 + RTEL1, | |
|---|---|---|---|---|---|---|---|---|
| | Vehicle | | 10 mg/kg | | 10 mg/kg | | 10 mg/kg | |
| Day | Mean | SEM | Mean | SEM | Mean | SEM | Mean | SEM |
| 0 | 0.000 | 0.124 | 0.000 | 0.059 | 0.000 | 0.204 | 0.000 | 0.086 |
| 7 | −0.064 | 0.118 | −0.018 | 0.057 | −0.052 | 0.189 | −0.084 | 0.080 |
| 14 | −0.016 | 0.137 | −0.007 | 0.068 | −0.085 | 0.200 | −0.116 | 0.086 |
| 21 | −0.073 | 0.145 | −0.128 | 0.083 | −0.199 | 0.188 | −0.255 | 0.093 |
| 28 | −0.066 | 0.135 | −0.118 | 0.092 | −0.220 | 0.204 | −0.359 | 0.094 |
| 35 | −0.083 | 0.138 | −0.166 | 0.090 | −0.244 | 0.187 | −0.428 | 0.085 |
| 42 | −0.091 | 0.131 | −0.187 | 0.086 | −0.254 | 0.165 | −0.458 | 0.083 |
| 49 | −0.146 | 0.138 | −0.259 | 0.088 | −0.317 | 0.164 | −0.550 | 0.067 |
| 56 | −0.117 | 0.148 | −0.308 | 0.100 | −0.346 | 0.177 | −0.608 | 0.069 |

TABLE 33
Serum HBsAg kinetic normalized to the vehicle control group (baseline corrected)

| | | | RTEL1 | | FUBP1 | | FUBP1 + RTEL1, | |
|---|---|---|---|---|---|---|---|---|
| | Vehicle | | 10 mg/kg | | 10 mg/kg | | 10 mg/kg | |
| Day | Mean | SEM | Mean | SEM | Mean | SEM | Mean | SEM |
| 0 | 0.000 | 0.175 | 0.000 | 0.137 | 0.000 | 0.239 | 0.000 | 0.151 |
| 7 | 0.000 | 0.167 | 0.046 | 0.131 | 0.012 | 0.223 | −0.021 | 0.143 |
| 14 | 0.000 | 0.194 | 0.009 | 0.153 | −0.069 | 0.242 | −0.100 | 0.162 |
| 21 | 0.000 | 0.204 | −0.055 | 0.167 | −0.126 | 0.238 | −0.182 | 0.172 |
| 28 | 0.000 | 0.191 | −0.052 | 0.163 | −0.154 | 0.244 | −0.292 | 0.164 |
| 35 | 0.000 | 0.195 | −0.083 | 0.164 | −0.161 | 0.233 | −0.345 | 0.162 |
| 42 | 0.000 | 0.185 | −0.096 | 0.157 | −0.163 | 0.211 | −0.368 | 0.155 |
| 49 | 0.000 | 0.195 | −0.113 | 0.164 | −0.171 | 0.214 | −0.404 | 0.153 |
| 56 | 0.000 | 0.210 | −0.192 | 0.179 | −0.229 | 0.231 | −0.491 | 0.163 |

TABLE 34
Serum HBeAg kinetic (baseline corrected)

| | | | FUBP1 | | RTEL1 | | FUBP1 + RTEL1, | |
|---|---|---|---|---|---|---|---|---|
| | Vehicle | | 10 mg/kg | | 10 mg/kg | | 10 mg/kg | |
| Day | Mean | SEM | Mean | SEM | Mean | SEM | Mean | SEM |
| 0 | 0 | 0.097277 | 0 | 0.076883 | 0 | 0.170624 | 0 | 0.081801 |
| 7 | −0.05643 | 0.100342 | 0.021962 | 0.062111 | −0.08878 | 0.177554 | −0.03 | 0.081588 |
| 14 | −0.00677 | 0.10343 | 0.067472 | 0.078954 | −0.07326 | 0.155543 | −0.02 | 0.084903 |
| 21 | −0.01767 | 0.102722 | 0.05285 | 0.082236 | −0.06093 | 0.153053 | −0.09 | 0.083717 |
| 28 | 0.018471 | 0.109088 | 0.021087 | 0.084894 | −0.11016 | 0.161684 | −0.12 | 0.083923 |
| 35 | −0.05957 | 0.101027 | −0.04855 | 0.083032 | −0.23351 | 0.136016 | −0.25 | 0.090098 |
| 42 | −0.04271 | 0.106042 | −0.09115 | 0.089527 | −0.13864 | 0.134806 | −0.22 | 0.076051 |
| 49 | −0.14716 | 0.106203 | −0.15171 | 0.098287 | −0.26485 | 0.143994 | −0.38 | 0.093478 |
| 56 | −0.1525 | 0.115082 | −0.17789 | 0.111281 | −0.27138 | 0.141619 | −0.41 | 0.088166 |

TABLE 35
Serum HBsAg kinetic normalized to the vehicle control group (baseline corrected)

| | | | FUBP1 | | RTEL1 | | FUBP1 + RTEL1, | |
|---|---|---|---|---|---|---|---|---|
| | Vehicle | | 10 mg/kg | | 10 mg/kg | | 10 mg/kg | |
| Day | Mean | SEM | Mean | SEM | Mean | SEM | Mean | SEM |
| 0 | 0.000 | 0.138 | 0.000 | 0.124 | 0.000 | 0.196 | 0.000 | 0.127 |
| 7 | 0.000 | 0.142 | 0.078 | 0.118 | −0.032 | 0.204 | 0.022 | 0.129 |
| 14 | 0.000 | 0.146 | 0.074 | 0.130 | −0.066 | 0.187 | −0.014 | 0.134 |
| 21 | 0.000 | 0.145 | 0.071 | 0.132 | −0.043 | 0.184 | −0.076 | 0.133 |
| 28 | 0.000 | 0.154 | 0.003 | 0.138 | −0.129 | 0.195 | −0.141 | 0.138 |
| 35 | 0.000 | 0.143 | 0.011 | 0.131 | −0.174 | 0.169 | −0.193 | 0.135 |
| 42 | 0.000 | 0.150 | −0.048 | 0.139 | −0.096 | 0.172 | −0.173 | 0.130 |
| 49 | 0.000 | 0.150 | −0.005 | 0.145 | −0.118 | 0.179 | −0.232 | 0.141 |
| 56 | 0.000 | 0.163 | −0.025 | 0.160 | −0.119 | 0.182 | −0.256 | 0.145 |

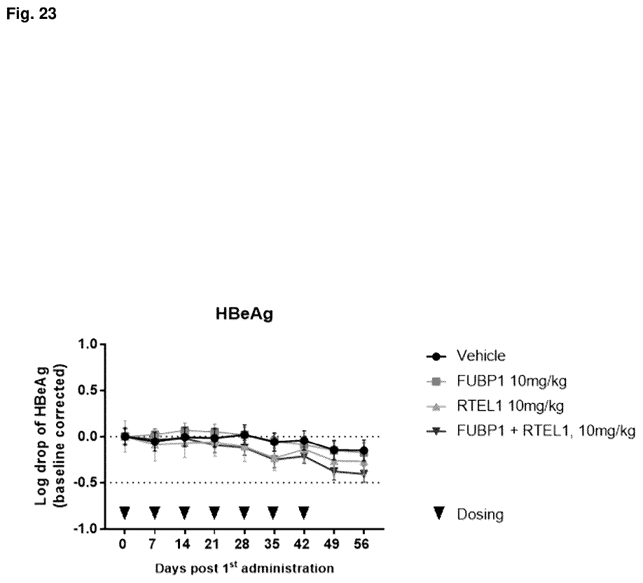

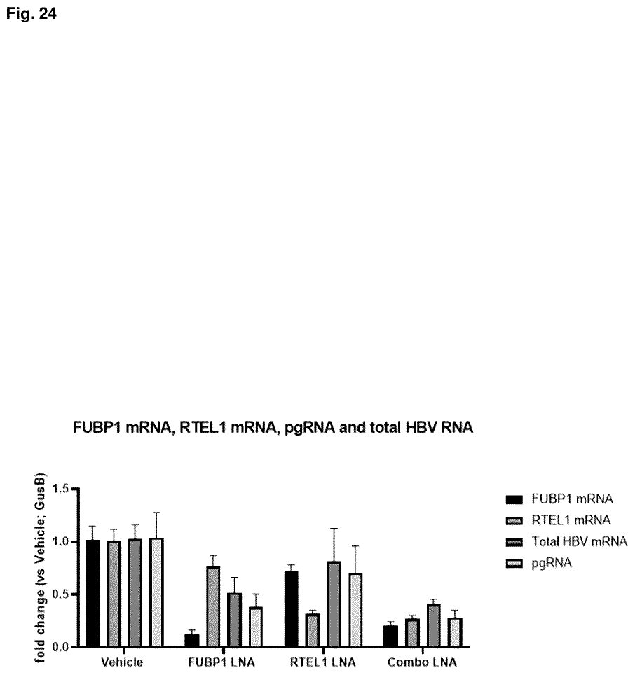

At end-point sacrifice the intrahepatic target engagement and the anti-viral efficacy of the LNAs on mRNA level was assessed by RT-qPCR (FIG. 24 and Table 36). FUBP1 LNA and RTEL1 LNA used should good target engagement in the liver mRNA samples. FUBP1 LNA reduced the FUBP1 mRNA by 88% in the single treatment and by 80% in the dual treatment arm. RTEL1 LNA was slightly less potent with RTLE1 mRNA being reduced by 69% in the single and 73% in the combination arms. Total HBV mRNA levels were reduced with all three treatment conditions. Here the strongest effects were seen with the FUBP1+RTEL1 LNA combination (59% reduction), followed by FUBP1 (48% reduction) alone and then by RTEL1 mono treatment (19% reduction). pgRNA levels were reduced even more and seem to follow the same trends for the three treatment conditions. Here the strongest impact is observed with the FUBP1+RTEL1 LNA combination (72% reduction), followed by FUBP1 (62% reduction) alone and then by RTEL1 mono treatment (29% reduction).

TABLE 36
Intrahepatic target engagement and efficacy
of RTEL1 and FUBP1 LNA molecules

| | FUBP1 mRNA | | | RTEL1 mRNA | | | Total HBV mRNA | | | pgRNA | | |
|---|---|---|---|---|---|---|---|---|---|---|---|---|
| | Mean | SEM | N | Mean | SEM | N | Mean | SEM | N | Mean | SEM | N |
| Vehicle | 1.015 | 0.060 | 5 | 1.012 | 0.049 | 5 | 1.028 | 0.060 | 5 | 1.040 | 0.106 | 5 |
| FUBP1 | 0.123 | 0.020 | 4 | 0.765 | 0.051 | 4 | 0.515 | 0.073 | 4 | 0.380 | 0.063 | 4 |
| LNA | | | | | | | | | | | | |
| RTEL1 | 0.720 | 0.033 | 3 | 0.314 | 0.020 | 3 | 0.810 | 0.183 | 3 | 0.705 | 0.149 | 3 |
| LNA | | | | | | | | | | | | |
| Combo | 0.203 | 0.018 | 5 | 0.268 | 0.015 | 5 | 0.409 | 0.020 | 5 | 0.280 | 0.032 | 5 |
| LNA | | | | | | | | | | | | |

### Example 4-In Vitro Testing of Combination Compounds in PHH

### Example 2.1—Primary Human Hepatocytes (PHH)

Fresh primary human hepatocytes (PHH) harvested from humanized mice (uPA/SCID mice)-herein called PHH-were obtained from PhoenixBio Co., Ltd (Japan) in 96-well format and cultured in modified hepatocyte clonal growth medium (dHCGM). dHCGM is a DMEM medium containing 100 U/ml Penicillin, 100 μg/ml Streptomycin, 20 mM Hepes, 44 mM NaHC03, 15 μg/ml L-proline, 0.25 μg/ml Insulin, 50 nM Dexamethasone, 5 ng/ml EGF, 0.1 mM Asc-2P, 2% DMSO and 10% FBS (Ishida et al., 2015). Cells were cultured at 37° C., in a humidified atmosphere with 5% CO2.

PHH were infected with HBV GtD derived from HepG2.2.15 cell culture in the presence of 4% PEG for 16-20 hours. The virus inoculum was removed the following day and cells were washed 3 times with PBS before addition of fresh medium. The LNA treatment started at day 4 post HBV infection in triplicates in a 1:3 dilution series in a total volume of 120 μl per well. The start concentration is mentioned in Table 37. The same treatment was repeated on day 11 and 18. On day 21, the supernatants were harvested and stored at −80° C. for further analysis of HBsAg, HBeAg and secreted HBV DNA. The cells were washed with 1×DPBS (Gibco, #14190250) once using 150 μl/well. For the RNA readout 200 μl/well MagNA Pure 96 External Lysis Buffer (Roche, #06374913001) was added and the plates were frozen at −80° C.

TABLE 37
Start concentration of tested LNAs

| | | | START |
|---|---|---|---|
| | | | CONCEN- |
| CMP ID NO | CONJUGATION | TARGET | TRATION |
| 335_1 | naked | scramble | 30 μM |
| 330_1 | naked | FUBP1 | 30 μM |
| 246_2 | naked | RTEL1 | 30 μM |
| 331_2 | naked | FUBP1 + RTEL1 | 15 μM |
| 331_1 | naked | FUBP1 + RTEL1 | 15 μM |
| 332_1 | naked | RTEL1 + FUBP1 | 15 μM |
| 330_1 + 246_2 | naked | FUBP1 + RTEL1 | 15 μM each |
| 332_2 | Naked | RTEL1 + FUBP1 | 15 μM |
| 335_1 | GalNAc | scramble | 10 μM |
| 326_3 | GalNAc | FUBP1 | 10 μM |
| 245_1 | GalNAc | RTEL1 | 10 μM |
| 333_1 | GalNAc | FUBP1 + RTEL1 | 5 μM |
| 334_1 | GalNAc | RTEL1 + FUBP1 | 5 μM |
| 326_3 + 245_1 | GalNAc | FUBP1 + RTEL1 | 5 μM |

### Example 2.2—Cell Viability Assay

The viability of the treated PHH cells was determined on Day 21 post-infection using the Cell Counting Kit-8 (CCK8) according to the manufacturer's instructions. The CCK8 mixture was prepared by adding the CCK8 reagent to the differentiation medium in a 1:10 ratio. Therefore the cell supernatant was removed and stored at −80° C. and 100 UL of the CCK8 mixture was added to each well and incubated at 37° C. for 1 hour. Absorbance at 450 nm was measured using an Envision reader (Perkin Elmer).

### Example 2.3—RNA Extraction and RT-qPCR

Total RNA was extracted from the cells using a MagNA Pure robot and the MagNA Pure 96 Cellular RNA Large Volume Kit (Roche, #05467535001) according to the manufacturer's protocol using the “Cellular RNA LV” protocol with a final elution volume of 50 μl.

The RTEL1 and FUBP1 mRNA, the pgRNA and intrahepatic DNA expression levels were quantified in technical duplicates by qPCR using a QuantStudio 12K Flex (Applied Biosystems), the TaqMan RNA-to-CT 1-Step Kit (Applied Biosystems, #4392938), and human GusB endogenous control. The qPCR was run on the QuantStudio Cycler with 48° C. for 15 minutes, then 95° C. for 10 minutes, then 40 cycles with 95° C. for 15 seconds and 60° C. for 1 minute, with 10 μl total reaction volume. The mRNA expression was analyzed using the comparative cycle threshold 2-ΔΔCt method normalized to the reference gene GusB and to HBV-infected, non-treated cells. TaqMan primers used for GusB mRNA, RTEL1 mRNA, FUBP1 mRNA, total intrahepatic HBV DNA and pgRNA quantification are listed in Table 38.

TABLE 38
Primers for GusB mRNA, RTEL1 mRNA, FUBP1 mRNA, total intrahepatic HBV DNA
and pgRNA quantification

| Target | Source |
|---|---|
| FUBP1 | ThermoFisher-Assay ID: Hs0090076_m1; FAM |
| RTEL1 | ThermoFisher-Assay ID: Hs02568623_s1; FAM |
| Total HBV DNA | ThermoFisher-Assay ID: Pa03453406_s1 P, S/P; FAM |
| pgRNA | ThermoFisher-Custom assay: |
| | Probe FAM GAGGCAGGTCCCCTAGAAGA (SEQ ID NO: 344) |
| | Forward GGAGTGTGGATTCGCACTCCT (SEQ ID NO: 345) |
| | Reverse AGATTGAGATCTTCTGCGAC (SEQ ID NO: 346) |
| GusB | ThermoFisher-Assay ID: Hs00939627 m1 |

### Example 2.4—Supernatant DNA Extraction and qPCR to Quantify Secreted HBV DNA

DNA was extracted from 40 μl of supernatant on the MagNA Pure robot with the MagNA Pure 96 DNA and Viral NA Small Volume Kit (Roche, #06543588001) according to the manufacturer's protocol using the “Viral NA Plasma ext lys SV 4.0” protocol with a final elution volume of 50 μl. For quantification of HBV DNA a 99 nucleotide fragment covering the core region was amplified with forward primer CTG TGC CTT GGG TGG CTT T (final concentration 200 nM), reverse primer AAG GAA AGA AGT CAG AAG GCA AAA (final concentration 200 nM), and probe 56-FAM-AGC TCC AAA/ZEN/TTC TTT ATA AGG GTC GAT GTC CAT G-3IABKFQ (final concentration 100 nM) (IDT DNA) using the TaqMan Gene Expression Master Mix and the following cycling condition: 2 minutes at 50° C., 10 minutes at 95° C., and 40 cycles of 95° C. for 15 seconds and 60° C. for 1 minutes. All qPCR reactions were performed using the QuantStudio 12K Flex Real Time PCR system (Life Technologies). The TaqMan primers used for secreted HBV DNA quantification are listed in Table 39.

The relative expression levels were calculated using the comparative cycle threshold 2-ACt method normalized to HBV-infected, non-treated cells.

TABLE 39
Primers for HBV from supernatant quantification

| Target | Source |
|---|---|
| HBV | forward primer CTG TGC CTT GGG TGG CTT T (SEQ ID NO: 353) |
| core | reverse primer AAG GAA AGA AGT CAG AAG GCA AAA (SEQ ID NO: 354) |
| | probe 56-FAM-AGC TCC AAA/ZEN/TTC TTT ATA AGG GTC GAT GTC CAT G- |
| | 3IABKFQ (SEQ ID NO: 355) |

### Example 2.5—HBsAG/HBeAG Chemiluminescence Immunoassay

HBsAg and HBeAg were determined in the supernatants harvested on day 21 post-infection using the HBsAg or HBeAg CLIA kit (DiaSino, #DS1877032012V4, #DS1877012012V4). The assay was performed according to the manufacturer's protocol and the supernatant was used in a 1:50 dilution. The luminescence signal was measured using an Envision reader (Perkin Elmer).

### Results

### Results 4.1: Reduction of HBV pgRNA in HBV Infected PHH Cells as a Functional Validation of the Knock-Down of RTEL1 and FUBP1 mRNA

The experimental set-up took advantage of the single molecules for RTEL1 and FUBP1, both molecules in combination, and as covalently linked molecules.

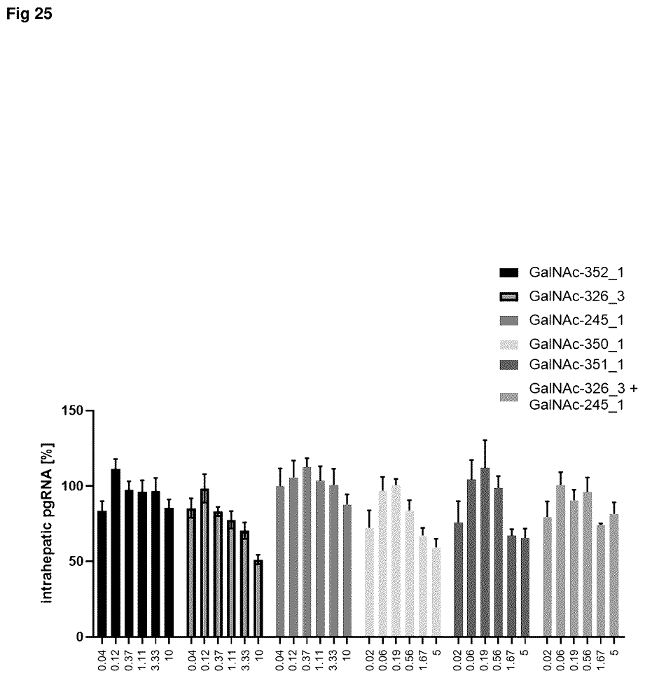

As can be derived from FIG. 25 the negative control (Gal-NAc-352_1) did not have an effect on the reduction of intrahepatic pgRNA. As a single treatment, the Gal-NAc-326 (FUBP1 ASO) reduced the pgRNA at the highest concentration to roughly 51% remaining pgRNA. Using the single treatment of Gal-NAc-245_1 (RTEL1 ASO) it was possible to observe a trend to HBV RNA reduction (i.e. ˜12%) at the highest 10 μM concentration. Combining the two single ASOs for RTEL1 and FUBP1 (Gal-NAc-245_1 and Gal-NAc-326_3 respectively) we saw a slight decrease in the maximum efficacy (i.e. 18-25%) of the reduction of HBV RNA as compared to the maximum efficacy obtained previously by the Gal-NAc-326 (i.e. 50%). Interestingly, both dual molecules showed concentration response effect on the pgRNA level: the dual-molecule Gal-NAc-350_1 showed a maximum efficacy at a 40% reduction of pgRNA. The second dual FUBP1/RTEL1 ASO Gal-NAc-351_1 also showed a reduction of pgRNA with a maximum efficacy at 34%.

### Results 4.2: Reduction of Intrahepatic Total HBV RNA in HBV Infected PHH Cells as a Functional Validation of the Knock-Down of RTEL1 and FUBP1 mRNA

The experimental set-up takes advantage of the single molecules for RTEL1 and FUBP1, both molecules in combination and as covalently linked molecules.

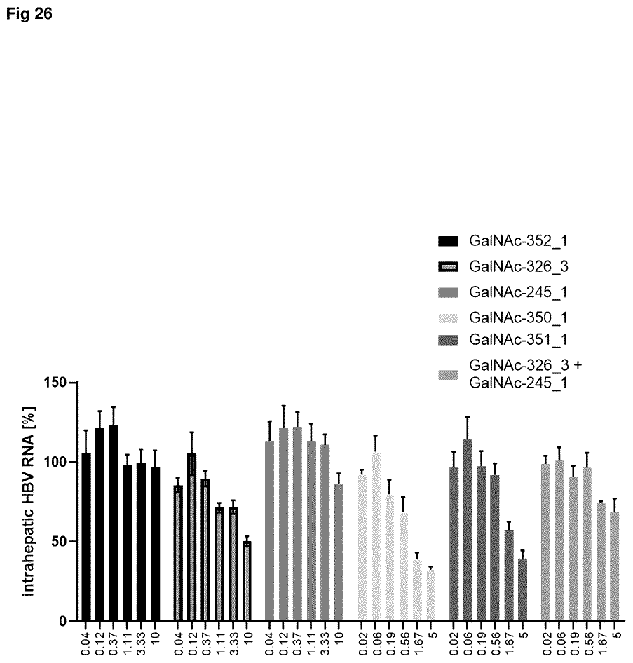

As indicated in FIG. 26 the negative control (Gal-NAc-352_1) did not have an effect on the reduction of intrahepatic HBV RNA. As a single treatment, the Gal-NAc-326 (FUBP1 ASO) reduced in a dose response manner HBV RNA with a maximum effect at the highest concentration (10 μM) of ˜50% inhibition of HBV RNA. Using the single treatment of Gal-NAc-245_1 (RTEL1 ASO) we observed a slight reduction of HBV RNA (˜14%) at the highest concentration (10 μM).

When the two single ASOs for RTEL1 and FUBP1 (Gal-NAc-245_1 and Gal-NAc-326 respectively) were combined in the same treatment, a dose response inhibition of the HBV RNA was also observed with a maximum efficacy (i.e. 37%) seen at the highest dose used for both compounds (5 μM). Surprisingly, linking the molecules covalently induced a stronger inhibition of the HBV RNA in a dose dependent manner with a maximum efficacy of 67% and 60% with GalNAc-350_1 and GalNAc-351_1 respectively.

Overall these results demonstrated that using covalently linked molecules can improve the reductions of intrahepatic HBV RNA as compared to the use of the single molecules targeting the RTEL1 and FUBP1 mRNA targets or the combination of the single ASOs

### Results 4.3: Reduction of mRNA Expression and Associated EC50

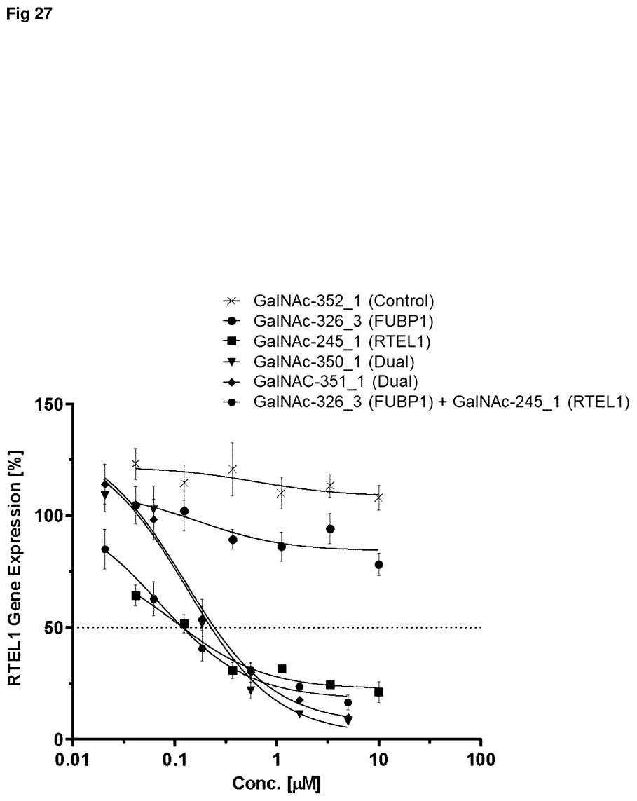

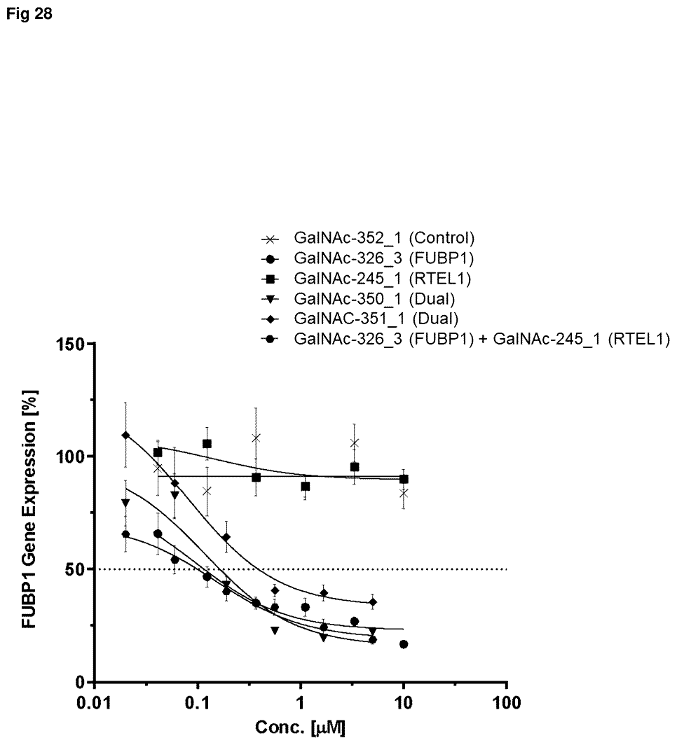

As can be derived from the Table 40 and FIG. 27 (RTEL1 knock down); and from the Table 41 and FIG. 28 (FUBP1 knock down), both dual compounds investigated (CMP ID NO: GalNAc-350_1 and GalNAC-351_1) reduce target mRNA expression with an EC50 of roughly 0.1 μM for both FUBP1 and RTEL1 mRNA targets. The effect of both dual compounds (i.e. covalently linked FUBP1 and RTEL1 ASOs) are comparable to the EC50s obtained using the individual ASO molecules for FUBP1 (EC50 for FUBP1 k.d is 0.095 μM) and RTEL1 (EC50 for RTEL1 k.d. is 0.10 μM). Thus it is possible to obtain similar k.d. of target mRNA with a single molecule having both ASO molecules covalently connected by a linker.

In summary, this demonstrates that the covalently linked dual ASO molecule exerts a comparable knock down of both target mRNAs (i.e. RTEL1 and FUBP mRNA) as compared to using the individual ASO molecules. In addition, the EC50 of the dual molecules is also comparable to the EC50s obtained when using a combination of the individual ASOs for FUBP1 and RTEL1.

TABLE 40
In vitro potency of dual RTEL1/FUBP1 ASOs,
single RTEL1 ASO, single FUBP1
ASO, and the combination of the single
ASOs in PXB-PHH cells. Target RTEL1 mRNA levels
are normalized to GUSB and calculated as % of control.
In the following table the individual
EC50 values of RTEL 1 target engagement can found:
RTEL1 knock-down EC50's

| Compound ID NO | EC50 RTEL1 k.d. |
|---|---|
| GalNAc-352_1 (negative control) | N/A |
| GalNAc-326_3 (FUBP1) | N/A |
| GalNAc-245_1 (RTEL1) | 0.10 uM |
| GalNAc-350_1 (dual) | 0.13 uM |
| GalNAC-351_1 (dual) | 0.12 uM |
| GalNAc-326_3 + GalNAc-245_1 | 0.07 uM |
| (FUBP1 and RTEL1 added | |
| as two individual ASOs) | |

TABLE 41
In vitro potency of dual RTEL1/FUBP1 ASOs,
single RTEL1 ASO, single FUBP1
ASO, and the combination of the single ASOs in
PXB-PHH cells. Target FUBP1 mRNA levels
are normalized to GUSB and calculated as % of control.
In the following table the individual
EC50 values of FUBP1 target engagement can found:
FUBP1 knock-down EC50's

| Compound # | EC50 FUBP1 k.d. |
|---|---|
| GalNAc-352_1 (negative control) | N/A |
| GalNAc-326_3 (FUBP1) | 0.095 uM |
| GalNAc-245_1 (RTEL1) | N/A |
| GalNAc-350_1 (dual) | 0.12 uM |
| GalNAC-351_1 (dual) | 0.082 uM |
| GalNAc-326_3 + GalNAc-245_1 | 0.15 uM |
| (FUBP1 and RTEL1 added as | |
| two individual ASOs) | |

### CONCLUSION

These results indicate that monotherapy with FUBP1 LNA and RTEL1 LNAs reduce the intrahepatic cccDNA burden, intrahepatic HBV transcriptional activity and HBV serum viremia incl HBV DNA, HBsAg and HBeAg. Furthermore, these results show that the combination of 5 FUBP1 LNA with RTLE1 LNA gives an additional benefit and leads to an even further reduction of viral load for cccDNA, HBV mRNA and serum viral readouts. For this reason FUBP1 and RTEL1 combination therapies may give added benefit over monotherapies and should be evaluated as potential combination strategies in chronic HBV patients.

1. A composition comprising an inhibitor of RTEL1 and an inhibitor of FUBP1.

2. A pharmaceutical composition comprising an inhibitor of RTEL1 and an inhibitor of FUBP1, or a pharmaceutically acceptable salt thereof, and a pharmaceutically acceptable diluent, solvent, carrier, salt and/or adjuvant.

3. A kit comprising an inhibitor of RTEL1 and an inhibitor of FUBP1.

4. The composition of claim 1, wherein the inhibitor of RTEL1 is capable of reducing covalently closed circular DNA (cccDNA) in an infected cell.

5. The composition of claim 1, wherein the RTEL1 inhibitor is an nucleic acid molecule of 12 to 60 nucleotides in length, 12 to 30 nucleotides in length, 12 to 25 nucleotides in length, or 15 to 21 nucleotides in length, comprising a contiguous nucleotide sequence of at least 10 nucleotides in length which is at least 80%, at least 85%, at least 90%, or at least 95% complementary to a mammalian RTEL1 target nucleic acid, wherein the nucleic acid molecule is capable of reducing the expression of RTEL1.

6. The composition of claim 5, wherein the mammalian RTEL1 target nucleic acid is selected from SEQ ID NO: 1 or 2.

7. The composition of claim 6, wherein the antisense oligonucleotide capable of reducing the expression of RTEL1 comprises:
(SEQ ID NO: 243)
AATTttacatactctgGT,
(SEQ ID NO: 244)
AAttttacatactctGGTC,
(SEQ ID NO: 245)
TTacatactctggtCAAA,
(SEQ ID NO: 246)
CTttattataactTgaAtCTC,
or
(SEQ ID NO: 246)
CTttattataacttgaaTCTC.
wherein capital letters are beta-D-oxy LNA nucleosides, lowercase letters are DNA nucleosides, all LNA C are 5-methyl cytosine, and all internucleoside linkages are phosphorothioate internucleoside linkages.

8. The composition of claim 1, wherein the inhibitor of FUBP1 is capable of reducing cccDNA and/or pre-genomic RNA (pgRNA) in an infected cell.

9. The composition of claim 1, wherein the FUBP1 inhibitor is a nucleic acid molecule of 12 to 60 nucleotides in length, 12 to 30 nucleotides in length, 12 to 25 nucleotides in length, or 15 to 21 nucleotides in length, which comprises a contiguous nucleotide sequence of 10 to 30 nucleotides in length, 12 to 25 nucleotides in length, or 15 to 21 nucleotides in length, wherein the contiguous nucleotide sequence is at least 80%, at least 85%, at least 90%, or at least 95% complementarity to a mammalian FUBP1 target nucleic acid, wherein the nucleic acid molecule is capable of inhibiting the expression of FUBP1.

10. The composition of claim 9, wherein the mammalian FUBP1 target nucleic acid is selected from the group consisting of SEQ ID NOs: 247-254.

11. The composition of claim 10, wherein the antisense oligonucleotide capable of inhibiting the expression of FUBP1 comprises:
(SEQ ID NO: 325)
CTTatGctttttatgGT,
(SEQ ID NO: 325)
CTTaTgctttttatgGT,
(SEQ ID NO: 326)
CTtATgctttttatgGTT,
(SEQ ID NO: 326)
CTtAtgctttttatgGTT,
(SEQ ID NO: 326)
CTtAtgctttttatGgTT,
(SEQ ID NO: 326)
CTtAtgctttttatGGTT,
(SEQ ID NO: 327)
GcttTttatggtTtCAC,
(SEQ ID NO: 328)
TATgcTttttatggtTTC,
(SEQ ID NO: 329)
AcCAAttttcatttCtAC,
or
(SEQ ID NO: 330)
CcccataaccataGTC
wherein capital letters are beta-D-oxy LNA nucleosides, lowercase letters are DNA nucleosides, all LNA C are 5-methyl cytosine, and all internucleoside linkages are phosphorothioate internucleoside linkages.

12. A method of treating or preventing a hepatitis B virus (HBV) infection in a subject, wherein the method comprises administering to the subject the composition of claim 1.

13. A method of treating or preventing a HBV infection in a subject, wherein the method comprises administering to the subject an inhibitor of RTEL1, wherein the method further comprises administering to the subject an inhibitor of FUBP1.

14. A method of treating or preventing a HBV infection in a subject, wherein the method comprises administering an inhibitor of FUBP1 to the subject, wherein the method further comprises administering to the subject an inhibitor of RTEL1.

15. A method of treating or preventing a HBV infection in a subject, wherein the method comprises administering to the subject a combination of an inhibitor of RTEL1 and an inhibitor of FUBP1.

16. The pharmaceutical composition of claim 2, wherein the inhibitor of RTEL1 is capable of reducing cccDNA in an infected cell.

17. The pharmaceutical composition of claim 2, wherein the RTEL1 inhibitor is an nucleic acid molecule of 12 to 60 nucleotides in length, 12 to 30 nucleotides in length, 12 to 25 nucleotides in length, or 15 to 21 nucleotides in length, comprising a contiguous nucleotide sequence of at least 10 nucleotides in length which is at least 80%, at least 85%, at least 90%, or at least 95% complementary to a mammalian RTEL1 target nucleic acid, wherein the nucleic acid molecule is capable of reducing the expression of RTEL1.

18. The pharmaceutical composition of claim 17 wherein the mammalian RTEL1 target nucleic acid is selected from SEQ ID NO: 1 or 2.

19. The pharmaceutical composition of claim 18, wherein the antisense oligonucleotide capable of reducing the expression of RTEL1 comprises:
(SEQ ID NO: 243)
AATTttacatactctgGT,
(SEQ ID NO: 244)
AAttttacatactctGGTC,
(SEQ ID NO: 245)
TTacatactctggtCAAA,
(SEQ ID NO: 246)
CTttattataactTgaAtCTC,
or
(SEQ ID NO: 246)
CTttattataacttgaaTCTC,
wherein capital letters are beta-D-oxy LNA nucleosides, lowercase letters are DNA nucleosides, all LNA C are 5-methyl cytosine, and all internucleoside linkages are phosphorothioate internucleoside linkages.

20. The pharmaceutical composition of claim 2, wherein the inhibitor of FUBP1 is capable of reducing cccDNA and/or pgRNA in an infected cell.

21. The pharmaceutical composition of claim 2, wherein the FUBP1 inhibitor is a nucleic acid molecule of 12 to 60 nucleotides in length, 12 to 30 nucleotides in length, 12 to 25 nucleotides in length, or 15 to 21 nucleotides in length, which comprises a contiguous nucleotide sequence of 10 to 30 nucleotides in length, 12 to 25 nucleotides in length, or 15 to 21 nucleotides in length, wherein the contiguous nucleotide sequence is at least 80%, at least 85%, at least 90%, or at least 95% complementarity to a mammalian FUBP1 target nucleic acid, wherein the nucleic acid molecule is capable of inhibiting the expression of FUBP1.

22. The pharmaceutical composition of claim 21, wherein the mammalian FUBP1 target nucleic acid is selected from the group consisting of SEQ ID NOs: 247-254.

23. The pharmaceutical composition of claim 22, wherein the antisense oligonucleotide capable of inhibiting the expression of FUBP1 comprises:
(SEQ ID NO: 325)
CTTatGctttttatgGT,
(SEQ ID NO: 325)
CTTaTgctttttatgGT,
(SEQ ID NO: 326)
CTtATgctttttatgGTT,
(SEQ ID NO: 326)
CTtAtgctttttatgGTT,
(SEQ ID NO: 326)
CTtAtgctttttatGgTT,
(SEQ ID NO: 326)
CTtAtgctttttatGGTT,
(SEQ ID NO: 327)
GcttTttatggtTtCAC,
(SEQ ID NO: 328)
TATgcTttttatggtTTC,
(SEQ ID NO: 329)
AcCAAttttcatttCtAC,
or
(SEQ ID NO: 330)
CcccataaccataGTC,
wherein capital letters are beta-D-oxy LNA nucleosides, lowercase letters are DNA nucleosides, all LNA C are 5-methyl cytosine, and all internucleoside linkages are phosphorothioate internucleoside linkages.
